SEQUENCE LISTING (1 sequence)

SEQ ID NO: 1 - protein, 353 aa, synthetic construct
GGPVRVGFYDIEGTLGKGNFAVVKLGRHRITKTEVAIKIIDKSQLDAVNLEKIYREVQIMKMLDHPHIIKLYQVMETKSMLYLVTEYAKNGEIFDYLANHGRLNESEARRKFWQILSAVDYCHGRKIVHRDLKAENLLLDNNMNIKIADFGFGNFFKSGELLATWCGSPPYAAPEVFEGQQYEGPQLDIWSMGVVLYVLVCGALPFDGPTLPILRQRVLEGRFRIPYFMSEDCEHLIRRMLVLDPSKRLTIAQIKEHKWMLIEVPVQRPVLYPQEQENEPSIGEFNEQVLRLMHSLGIDQQKTIESLQNKSYNHFAAIYFLLVERLKSHRSSFPVEQRLLVPRGSDYKDDDDK

(19) United States
(12) Patent Application Publication
(10) Pub. No.: US 2026/0184722 A1
(43) Pub. Date: Jul. 2, 2026

(54) SIK2 MODULATORS AND USES THEREOF

(71) Applicant: NIMBUS SALACIA, INC., Boston, MA (US)

(72) Inventors: Derun Li, Boston, MA (US); Leela Dodda, Boston, MA (US); Scott D. Edmondson, Boston, MA (US); Nathan E. Genung, Boston, MA (US); Alan Graves, Boston, MA (US); Neelu Kaila, Boston, MA (US); Daniel Joseph Price, Boston, MA (US); Ivan V. Sergeyer, Boston, MA (US); Md Munan Shaik, Boston, MA (US); Sekhar Surapaneni, Somerset, NJ (US); Sebastien Campos, Hoddesdon (GB); Russell George Dushin, Mystic, CT (US); Bruce Lefker, Mystic, CT (US); Steffen Gressies, Berlin (DE); Nikolay Sitnikov, Berlin (DE)

(21) Appl. No.: 19/436,154

(22) Filed: Dec. 30, 2025

Related U.S. Application Data
(60) Provisional application No. 63/740,684, filed on Dec. 31, 2024.
(60) Provisional application No. 63/775,610, filed on Mar. 21, 2025.
(60) Provisional application No. 63/895,804, filed on Oct. 8, 2025.

(51) Int. Cl. C07D 491/22; A61K 31/437; A61K 31/444; A61K 31/4709; A61K 31/473; A61K 31/4985; A61K 31/501; A61K 31/5025; A61K 31/506; A61K 31/5383; A61K 31/553; C07B 59/00; C07D 498/22; C07D 519/00

(52) U.S. Cl. CPC C07D 491/22; A61K 31/437; A61K 31/444; A61K 31/4709; A61K 31/473; A61K 31/4985; A61K 31/501; A61K 31/5025; A61K 31/506; A61K 31/5383; A61K 31/553; C07B 59/002; C07D 498/22; C07D 519/00; C07B 2200/05

(57) ABSTRACT

The present invention provides compounds of formula I, compositions thereof, and methods of using the same for the inhibition of SIK2 and the treatment of SIK2-mediated disorders.

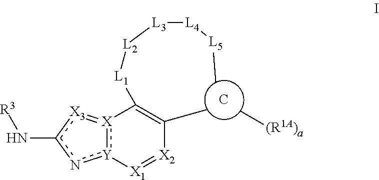

### SEQUENCE LISTING

This application contains a Sequence Listing which has been submitted in .xml format via EFS and is hereby incorporated by reference. The ST.26 copy, created on Dec. 9, 2025, is named 40102_SeqListing.xml and is 2,216 bytes in size.

### TECHNICAL FIELD OF THE INVENTION

The present invention relates to compounds and methods useful for inhibiting salt-inducible kinase 2 (SIK2). The invention also provides pharmaceutically acceptable compositions comprising compounds of the present invention and methods of using said compositions in the treatment of various disorders.

### BACKGROUND OF THE INVENTION

A protein kinase is an enzyme that catalyzes the transfer of phosphate groups to proteins or other organic molecules. Consequently, defective control of protein phosphorylation leads to uncontrolled signaling is involved in a number of diseases, including for example, inflammation, allergies, cancer, autoimmune diseases, CNS disorders, and angiogenesis.

The salt-inducible kinases (SIKs) are Ser/Thr kinases members of the adenosine monophosphate-activated kinase (AMPK) subfamily of kinases and three isoforms have been described, i.e., SIK1, SIK2 (QIK), and SIK3 (QSK). The SIKs play a number of roles in different cell types have been found to phosphorylate a number of substrates including CREB-responsive transcriptional co-activator (CRTC) proteins, and also histone de-acetylase (HDAC) proteins, thereby regulating the transcription of a number of different genes.

For example, SIK2 is known to play a role in the secretion of high levels of anti-inflammatory cytokines such as interleukin-10 (IL-10), very low levels of pro-inflammatory cytokines such as TNF-α, and in the regulation of IFNγ and IL-12 signaling, suggesting SIK2 may be an interesting target for inflammatory diseases. See e.g., Darling, et al, Biochem. J. 2017, 474(4):521-537 and Yao, et al., Nat. comm. 2013, 4(1):1-15.

The lack of selectivity towards salt inducible kinase isoforms, and in particular towards SIK2 may be a possible limitation in the use of such compounds. The aim of the present invention is to provide new compounds which are potent SIK2 inhibitors, and which also show selectivity over SIK1 and SIK3. There is a need for selective small molecule kinase inhibitors that inhibit SIK2.

### SUMMARY OF THE INVENTION

It has now been found that compounds of this invention, and pharmaceutically acceptable compositions thereof, are effective as inhibitors of SIK2. In certain embodiments, the invention provides for compounds of the formulae presented herein.

In one aspect, the disclosure provides a compound of formula I, or a pharmaceutically acceptable salt thereof:

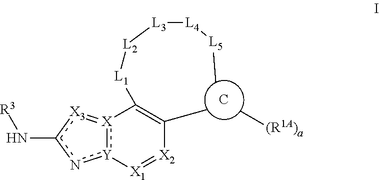

wherein X, Y, X₁, X₂, X₃, R^{1A}, R³, L₁, L₂, L₃, L₄, L₅, a, and Ring C are as described and defined herein.

Compounds of the present invention, and pharmaceutically acceptable compositions thereof, are useful for treating a variety of diseases, disorders or conditions, associated with modulating SIK2. Such diseases, disorders, or conditions include those described herein. Compounds provided by this invention are also useful for the study of SIK2 kinase in biological and pathological phenomena; the study of intracellular signal transduction pathways occurring in bodily tissues; and the comparative evaluation of new SIK2 inhibitors or other regulators of kinases, signaling pathways, and cytokine levels in vitro or in vivo.

### DETAILED DESCRIPTION OF CERTAIN EMBODIMENTS

### 1. General Description of Certain Embodiments of the Invention

In certain aspects, the present invention provides a compound of formula I:

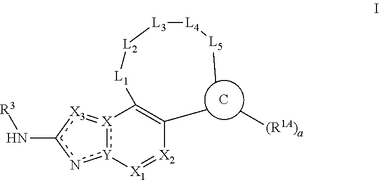

or a pharmaceutically acceptable salt thereof, wherein:
X is N or C;
Y is N or C, where when one of X and Y is N and the other of X and Y is C;
Each [symbol] is a single or double bond;
L₁ is selected from a bond, —O—, —S—, —S(O)₂—, —NR¹—, —C(O)—, —C(O)NR¹—, —NR¹C(O)—, —NR¹C(O)NR¹—, and —[C(R^{1a})(R^{1b})]₁₋₂—;
L₂ is selected from a bond, a substituted or unsubstituted alkenylene, —O—, —S—, —S(O)₂—, —N═S(O)(CH₃)—, —S(O)(CH₃)═N—, —S(O)₂NR¹—, —NR¹S(O)₂—, —NR¹—, —C(O)—, —C(O)NR¹—, —NR¹C(O)—, —NR¹C(O)NR¹—, an optionally substituted C₃-C₆ cycloalkyl, an optionally substituted C₃-C₆ heterocycloalkyl (having 1-4 heteroatoms independently selected from nitrogen, oxygen, and sulfur), an optionally substituted 5-6 membered monocyclic heteroaryl ring having 1-3 heteroatoms independently selected from nitrogen, oxygen, and sulfur; and —[C(R^{2a})(R^{2b})]₁₋₂—;
L₃ is selected from a bond, a substituted or unsubstituted alkenylene, —O—, —S—, —S(O)₂—, —N═S(O)(CH₃)—, —S(O)(CH₃)═N—, —S(O)₂NR¹—, —NR¹S(O)₂—, —NR¹—, —C(O)—, —C(O)NR¹—, —NR¹C(O)—, —NR¹C(O)NR¹—, an optionally substituted C₃-C₆ cycloalkyl, an optionally substituted C₃-C₆ heterocycloalkyl (having 1-4 heteroatoms independently selected from nitrogen, oxygen, and sulfur), an optionally substituted 5-6 membered monocyclic heteroaryl ring having 1-3 heteroatoms independently selected from nitrogen, oxygen, and sulfur; and —[C(R^{3a})(R^{3b})]₁₋₂—;
L₄ is selected from a bond, a substituted or unsubstituted alkenylene, —O—, —S—, —S(O)₂—, —N═S(O)(CH₃)—, —S(O)(CH₃)═N—, —S(O)₂NR¹—, —NR¹S(O)₂—, —NR¹—, —C(O)—, —C(O)NR¹—, —NR¹C(O)—, —NR¹C(O)NR¹—, an optionally substituted C₃-C₆ cycloalkyl, an optionally substituted C₃-C₆ heterocycloalkyl (having 1-4 heteroatoms independently selected from nitrogen, oxygen, and sulfur), an optionally substituted 5-6 membered monocyclic heteroaryl ring having 1-3 heteroatoms independently selected from nitrogen, oxygen, and sulfur; and —[C(R^{4a})(R^{4b})]₁₋₂—;
L₅ is selected from a bond, —O—, —S—, —S(O)₂—, —NR¹—, —C(O)—, —C(O)NR¹—, —NR¹C(O)—, —NR¹C(O)NR¹—, and —[C(R^{5a})(R^{5b})]₁₋₂—;
Each R¹ is independently selected from the group consisting of hydrogen, C₁₋₃ haloalkyl, C₁₋₃ hydroxyalkyl, C₀₋₃ alkyl-C₃-C₆ cycloalkyl, C₀₋₃ alkyl-C₃-C₆ heterocycloalkyl (having 1-4 heteroatoms independently selected from nitrogen, oxygen, and sulfur), and C₁₋₃ alkyl; wherein C₀₋₃ alkyl-C₃-C₆ cycloalkyl, C₀₋₃ alkyl-C₃-C₆ heterocycloalkyl and C₁₋₃ alkyl are substituted with y instances of R^{B};
   each R^{B} is independently halogen, C₁₋₃ alkyl, C₁₋₃ hydroxyalkyl, —CN, oxo, —OR, or —NR₂;
   each y is independently 0, 1, 2, or 3;
R^{1a}, R^{1b}, R^{2a}, R^{2a}, R^{3a}, R^{3b}, R^{4a}, R^{4b}, R^{5a}, and R^{5b} are independently selected from the group consisting of hydrogen, halogen, hydroxyl, N(R¹)₂, C₁₋₃ alkyl, —CN, C₁₋₃ alkoxy, C₀₋₃ alkyl-C₃-C₆ cycloalkyl, C₀₋₃ alkyl-C₃-C₆ heterocycloalkyl, and C₁₋₃ hydroxyalkyl, and C₁₋₃ haloalkyl, wherein C₀₋₃ alkyl-C₃-C₆ cycloalkyl, C₀₋₃ alkyl-C₃-C₆ heterocycloalkyl and C₁₋₃ alkyl are substituted with y instances of R^{B}; or
R^{1a} and R^{1b}, R^{2a} and R^{2b}, R^{3a} and R^{3b}, R^{4a} and R^{4b}, or R^{5a} and R^{5b}, and the carbon atom to which they are attached may combine to form:
   a) a 3-7 membered saturated or partially unsaturated bivalent monocyclic carbocyclylene or 4-7 membered saturated or partially unsaturated bivalent heterocyclylene ring (having 1-4 heteroatoms independently selected from nitrogen, oxygen, and sulfur, and 0 or 1 nitrogen atoms in addition to the 1-4 heteroatoms)—substituted with y instances of R^{B}, or
   b) a 4-12 membered saturated or partially unsaturated bivalent bicyclic ring system that is fused, bridged, or spirocyclic selected from carbocyclylene or heterocyclylene (having 1-4 heteroatoms independently selected from nitrogen, oxygen, and sulfur)—substituted with y instances of R^{B}, or
R^{1a} and R^{2a}, R^{1a} and R^{3a}, R^{2a} and R^{3a}, R^{2a} and R^{4a}, R^{3a} and R^{4a}, R^{3a} and R^{5a}, or R^{4a} and R^{5a}, and the atoms to which they are connected may combine to form:
   a) a 3-7 membered saturated or partially unsaturated bivalent monocyclic carbocyclylene or a 4-7 membered saturated or partially unsaturated bivalent heterocyclylene ring (having 1-4 heteroatoms independently selected from nitrogen, oxygen, and sulfur, and 0 or 1 nitrogen atoms in addition to the 1-4 heteroatoms)—substituted with y instances of R^{B}, or
   b) a 4-12 membered saturated or partially unsaturated bivalent bicyclic ring system that is fused, bridged, or spirocyclic selected from carbocyclylene or heterocyclylene (having 1-4 heteroatoms independently selected from nitrogen, oxygen, and sulfur)—substituted with y instances of R^{B};
X₁ and X₂ are each selected from N and CR² provided when Y is N, X₁ is CR²;
each R² is independently selected from hydrogen, OH, C₁₋₃ alkyl, C₁₋₃ alkoxy, and halogen;
X₃ is selected from O, S, N, NR⁷, and CR², provided when X₃ is selected from O, S, and NR⁷, X and Y are C;
R⁷ is hydrogen or C₁₋₃ alkyl;
Ring C is a 5-6 membered monocyclic heteroaryl ring having 1-3 heteroatoms independently selected from nitrogen, oxygen, and sulfur; an 8-10 membered bicyclic heteroaryl ring having 1-4 heteroatoms independently selected from nitrogen, oxygen, and sulfur; or a 7-12 membered saturated or partially unsaturated bicyclic heterocyclyl ring having 1-4 heteroatoms independently selected from nitrogen, oxygen, and sulfur;
each R^{1A} is independently halogen, —CN, oxo, —OR, —SR, —NR₂, —SO₂R, —SO₂NR₂, —S(O)R, —S(O)NR₂, —C(O)R, —CO₂R, —C(O)NR₂, —C(O)N(R)OR, —OC(O)R, —OC(O)NR₂, —N(R)CO₂R, —N(R)C(O)R, —N(R)C(O)NR₂, —N(R)C(NR)R, —N(R)C(NR)NR₂, —N(R)NR₂, —N(R)SO₂NR₂, —N(R)SO₂R, —N═S(O)R₂, —S(NR)(O)R, —N(R)S(O)R, —N(R)CN, —P(O)(OR)₂, —P(O)R₂, C₁-C₆ haloalkyl, C₁₋₃ hydroxyalkyl, C₃-C₆ halocycloalkyl, C₁-C₆ haloalkoxy, or an optionally substituted group selected from C₁₋₆ aliphatic; phenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; and a 5-6 membered monocyclic heteroaryl ring having 1-4 heteroatoms independently selected from nitrogen, oxygen, and sulfur;
a is 0, 1, 2, or 3;
R³ is —C(O)-L₆, —S(O)₂-L₆, an optionally substituted C₁₋₆ aliphatic, an optionally substituted C₁₋₆ aliphatic substituted with

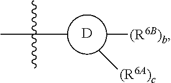

an optionally substituted 5-6 membered monocyclic heteroaromatic ring having 1-4 heteroatoms independently selected from nitrogen, oxygen, and sulfur, or

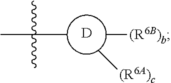

L₆ is selected from the group consisting of

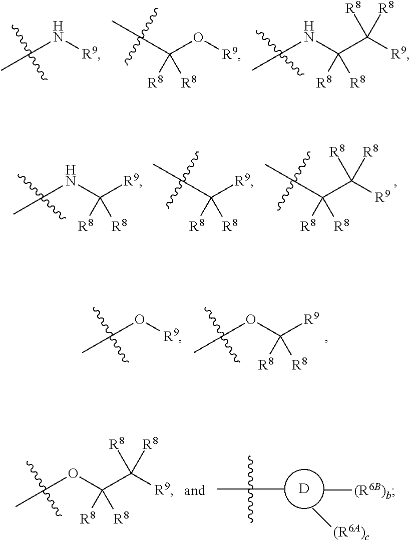

Ring D is a 3-8 membered saturated or partially unsaturated monocyclic carbocyclic, a 3-8 membered saturated or partially unsaturated monocyclic heterocyclic ring having 1-2 heteroatoms independently selected from nitrogen, oxygen, and sulfur, a 5-12 membered saturated or partially unsaturated bicyclic heterocyclic ring that is optionally bridged or spirocyclic having 1-4 heteroatoms independently selected from nitrogen, oxygen, and sulfur, or a 7-14 membered saturated or partially unsaturated tricyclic heterocyclic ring that is optionally bridged or spirocyclic having 1-4 heteroatoms independently selected from nitrogen, oxygen, and sulfur;
Each R⁸ is independently selected from hydrogen, halogen, hydroxyl, —CN, C₁₋₄ alkoxy, a 3-8 membered saturated or partially unsaturated monocyclic carbocyclic, C₁-C₆haloalkyl, and C₁₋₄ alkyl; or two R⁸ groups and the atom to which they are attached form oxo, a 3-8 membered saturated or partially unsaturated monocyclic carbocyclic ring or a 3-8 membered saturated or partially unsaturated monocyclic heterocyclic ring having 1-4 heteroatoms independently selected from nitrogen, oxygen, and sulfur;
R⁹ is selected from hydrogen, C₁-C₆ alkyl, C₁-C₆ haloalkyl, C₁₋₃ hydroxyalkyl, C₁-C₆ alkoxy, C₁-C₆ haloalkoxy, and an optionally substituted C₁₋₆ aliphatic; or
R⁹ is a cyclic group or an optionally substituted C₁₋₆ aliphatic substituted with a cyclic group, wherein the cyclic group is selected from:
   phenyl;
   a 3-8 membered saturated or partially unsaturated monocyclic carbocyclic ring;
   a 3-8 membered saturated or partially unsaturated monocyclic heterocyclic ring having 1-2 heteroatoms independently selected from nitrogen, oxygen, and sulfur;
   a 5-6 membered monocyclic heteroaromatic ring having 1-4 heteroatoms independently selected from nitrogen, oxygen, and sulfur;
   an 8-10 membered bicyclic heteroaromatic ring having 1-5 heteroatoms independently selected from nitrogen, oxygen, and sulfur;
   a 7-12 membered saturated or partially unsaturated bicyclic heterocyclic ring having 1-4 heteroatoms independently selected from nitrogen, oxygen, and sulfur;
   a 5-12 membered saturated or partially unsaturated bicyclic carbocyclic ring that is optionally bridged or spirocyclic; and
   a 5-12 membered saturated or partially unsaturated bicyclic heterocyclic ring that is optionally bridged or spirocyclic having 1-4 heteroatoms independently selected from nitrogen, oxygen, and sulfur,
   wherein the cyclic group is substituted with y instances of R^{A};
each R^{6A} is independently a C₁₋₆ aliphatic-cyclic or cyclic group, wherein the cyclic group is selected from:
   a 5-6 membered monocyclic heteroaromatic ring having 1-4 heteroatoms independently selected from nitrogen, oxygen, and sulfur;
   an 8-10 membered bicyclic heteroaromatic ring having 1-5 heteroatoms independently selected from nitrogen, oxygen, and sulfur;
   a 7-12 membered saturated or partially unsaturated bicyclic heterocyclic ring having 1-4 heteroatoms independently selected from nitrogen, oxygen, and sulfur;
   a 5-12 membered saturated or partially unsaturated bicyclic carbocyclic ring that is optionally bridged or spirocyclic; and
   a 5-12 membered saturated or partially unsaturated bicyclic heterocyclic ring that is optionally bridged or spirocyclic having 1-4 heteroatoms independently selected from nitrogen, oxygen, and sulfur,
   wherein R^{6A} is substituted with y instances of R^{A};
each R^{6B} is independently halogen, —CN, oxo, —OR, —SR, —NR₂, —SO₂R, —SO₂NR₂, —S(O)R, —S(O)NR₂, —C(O)R, —CO₂R, —C(O)NR₂, —C(O)N(R)OR, —OC(O)R, —OC(O)NR₂, —N(R)CO₂R, —N(R)C(O)R, —N(R)C(O)NR₂, —N(R)C(NR)R, —N(R)C(NR)NR₂, —N(R)NR₂, —N(R)SO₂NR₂, —N(R)SO₂R, —N═S(O)R₂, —S(NR)(O)R, —N(R)S(O)R, —N(R)CN, —P(O)(OR)₂, —P(O)R₂, or an optionally substituted group selected from C₁₋₆ aliphatic; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; and a 5-6 membered monocyclic heteroaryl ring having 1-4 heteroatoms independently selected from nitrogen, oxygen, and sulfur;
b is 0, 1, 2, or 3;
c is 0, 1, 2, or 3;
each R^{A} is independently selected from deuterium, a C₁₋₆ aliphatic group substituted with 0, 1, 2, or 3 groups independently selected from halogen, deuterium, —CN, —NR₂, and —OR; a 5-6 membered monocyclic heteroaromatic ring having 1-4 heteroatoms independently selected from nitrogen, oxygen, and sulfur substituted with 0, 1, 2, or 3 groups independently selected from halogen, C₁₋₃ alkyl, C₁₋₃ haloalkyl, —CN, —NR₂, and —OR; a 3-8 membered saturated or partially unsaturated monocyclic heterocyclic ring having 1-2 heteroatoms independently selected from nitrogen, oxygen, and sulfur substituted with 0, 1, 2, or 3 groups independently selected from halogen, C₁₋₃ alkyl, C₁₋₃ haloalkyl, —CN, —NR₂, and —OR; a 3-8 membered saturated or partially unsaturated monocyclic carbocyclic ring substituted with 0, 1, 2, or 3 groups independently selected from halogen, C₁₋₃ alkyl, C₁₋₃ haloalkyl, —CN, —NR₂, and —OR; a 4-12 membered saturated or partially unsaturated bicyclic ring system that is fused, bridged, or spirocyclic selected from carbocyclyl and heterocyclyl (having 1-4 heteroatoms independently selected from nitrogen, oxygen, and sulfur), wherein said carbocyclyl or heterocyclyl is substituted with 0, 1, 2, or 3 groups independently selected C₁₋₃ alkyl, C₁₋₃ alkoxy, C₁₋₃ haloalkyl, C₁₋₃ hydroxyalkyl, halogen, —CN, —OR, and —NR₂; halogen; —CN; —NO₂; —OR; —SR; —NR₂; —S(O)₂R; —S(O)₂NR₂; —S(O)R; —S(O)NR₂; —C(O)R; —C(O)OR; —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —S(NR)(O)R; —N(R)S(O)₂NR₂; and —N(R)S(O)₂R; or
two R^{A} on the same atom together form oxo or a cyclic group selected from:
   3-8 membered saturated or partially unsaturated monocyclic carbocyclic ring; and
   a 3-8 membered saturated or partially unsaturated monocyclic heterocyclic ring having 1-2 heteroatoms independently selected from nitrogen, oxygen, and sulfur;
wherein the cyclic group formed by two R^{A} on the same atom is substituted with 0, 1, 2, or 3 groups independently selected from a C₁₋₆ aliphatic group substituted with 0, 1, 2, or 3 groups independently selected from halogen, —CN, —NR₂, and —OR; halogen; —CN; —NO₂; —OR; —SR; —NR₂; —S(O)₂R; —S(O)₂NR₂; —S(O)R; —S(O)NR₂; —C(O)R; —C(O)OR; —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —N(R)S(O)₂NR₂; and —N(R)S(O)₂R; or
two R^{A} on adjacent atoms form a cyclic group selected from:
   3-8 membered saturated or partially unsaturated monocyclic carbocyclic ring;
   a 3-8 membered saturated or partially unsaturated monocyclic heterocyclic ring having 1-2 heteroatoms independently selected from nitrogen, oxygen, and sulfur;
   phenyl; and
   a 5-6 membered heteroaryl ring having 1-4 heteroatoms independently selected from nitrogen, oxygen, and sulfur,
wherein the cyclic group formed by two R^{A} on adjacent atoms is substituted with 0, 1, 2, or 3 groups independently selected from a C₁₋₆ aliphatic group substituted with 0, 1, 2, or 3 groups independently selected from halogen, —CN, —NR₂, and —OR; halogen; —CN; —NO₂; —OR; —SR; —NR₂; —S(O)₂R; —S(O)₂NR₂; —S(O)R; —S(O)NR₂; —C(O)R; —C(O)OR; —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —N(R)S(O)₂NR₂; and —N(R)S(O)₂R;
each R is independently hydrogen, —C(O)N(CH₃)₂, —C(O)₂CH₃, —C(O)₂C(CH₃)₃, —C(O)₂CH(CH₃)₂, —S(O)₂CH₃, an optionally substituted C₁₋₆ aliphatic group, an optionally substituted cyclic group, or an optionally substituted C₁₋₆ alkyl-cyclic group, wherein the cyclic group is selected from:
   an optionally substituted phenyl;
   an optionally substituted 3-7 membered saturated or partially unsaturated carbocyclic ring;
   an optionally substituted 3-7 membered saturated or partially unsaturated heterocyclic ring having 1-2 heteroatoms independently selected from nitrogen, oxygen, and sulfur;
   an optionally substituted 5-6 membered heteroaryl ring having 1-4 heteroatoms independently selected from nitrogen, oxygen, and sulfur;
   an optionally substituted 8-10 membered bicyclic heteroaryl ring having 1-4 heteroatoms independently selected from nitrogen, oxygen, and sulfur;
   an optionally substituted 7-12 membered saturated or partially unsaturated bicyclic heterocyclic ring having 1-4 heteroatoms independently selected from nitrogen, oxygen, and sulfur;
   an optionally substituted 5-8 membered saturated or partially unsaturated bridged bicyclic ring having 0-3 heteroatoms independently selected from nitrogen, oxygen, and sulfur;
   an optionally substituted 6-10 membered saturated or partially unsaturated spirocyclic ring having 0-3 heteroatoms independently selected from nitrogen, oxygen, and sulfur; and
   an optionally substituted 6-11 membered saturated or partially unsaturated bicyclic ring having 0-2 heteroatoms independently selected from nitrogen, oxygen, and sulfur; and/or
two R groups on the same atom are taken together with the same atom to form a cyclic group selected from:
   an optionally substituted 4-7 membered saturated or partially unsaturated carbocyclyl;
   an optionally substituted 4-7 membered saturated or partially unsaturated heterocyclyl having 1-3 heteroatoms independently selected from nitrogen, oxygen, and sulfur; and
   an optionally substituted 5-6 membered heteroaryl ring having 1-3 heteroatoms independently selected from nitrogen, oxygen, and sulfur
wherein the optionally substituted R moiety is substituted with 0, 1, 2, 3, or 4 R^{Sub};
   each R^{Sub} is independently selected from C₁₋₆ aliphatic group substituted with 0, 1, 2, or 3 groups independently selected from halogen, —CN, —N(R^{N})₂, and —O R^{N}; halogen; —CN; —NO₂; —OR^{N}; —SR^{N}; —N(R^{N})₂; —S(O)₂R^{N}; —S(O)₂N(R^{N})₂; —S(O)R^{N}; —S(O)N(R^{N})R₂; —C(O)R^{N}; —C(O)OR^{N}; —C(O)N(R^{N})₂; —C(O)N(R^{N})OR^{N}; —OC(O)R^{N}; —OC(O)N(R^{N})₂; —N(R^{N})C(O)OR^{N}; —N(R^{N})C(O)R^{N}; —N(R^{N})C(O)N(R^{N})₂; —N(R^{N})C(NRN)N(R^{N})₂; —N(R^{N})S(O)₂N(R^{N})₂; and —N(R^{N})S(O)₂R^{N}; and each R^{N} is independently selected from H and C₁₋₃ alkyl.

In certain other aspects, the present invention provides a compound of formula I wherein said compound is of formula I-a, I-b, or I-c:

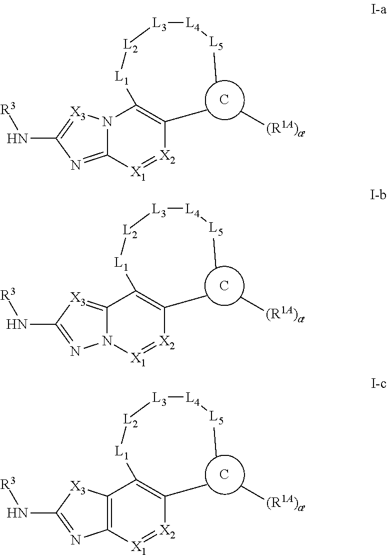

or a pharmaceutically acceptable salt thereof, wherein each of X₁, X₂, X₃, R^{1A}, R³, L₁, L₂, L₃, L₄, L₅, a, and Ring C are as defined herein, both singly and in combination.

In some embodiments, the present invention provides a pharmaceutical composition comprising a compound of formula I or formula I-a, I-b, or I-c and a pharmaceutically acceptable carrier, adjuvant, or diluent.

In some embodiments, the present invention provides a method of treating a SIK2-mediated disease, disorder, or condition comprising administering to a patient in need thereof, a compound of formula I or formula I-a, I-b, or I-c, or a pharmaceutically acceptable salt thereof.

### 2. Compounds and Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001.

Compound structures shown throughout the present specification and in the examples or claims contain designations at certain stereocenters which indicate the following: “or1” is intended to cover stereochemically pure compounds wherein the stereochemistry at the stereocenter marked with “or1” is either the stereochemistry shown in the diagram or wherein the marked stereocenter has a configuration opposite to what is shown in the diagram. In structures with stereocenters with the same label such as “or1” the relative stereochemistry between two stereocenters with said label is as drawn, as in compounds I-36 and I-37

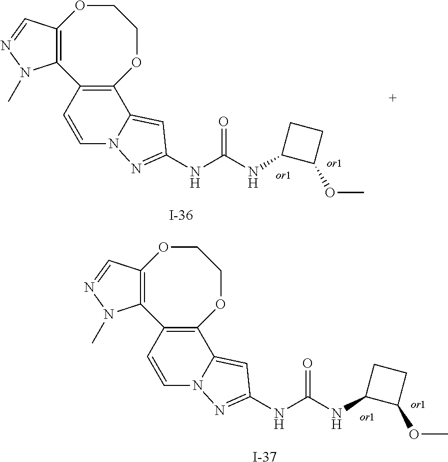

Stereocenters marked with “abs” intend to cover material wherein the marked stereocenter is of the stereochemistry shown in the diagram. Stereocenters marked with “&1” or “and1” indicate that the compound material has a mixture of R and S-configured stereoisomers with respect to the marked stereocenter and is in the same relative configuration to each other if they share the same label such as “and1” or “&1”.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-12 carbon atoms. In some embodiments, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur Unless otherwise specified, a bridged bicyclic group is optionally substituted with one or more substituents as set forth for aliphatic groups. Additionally or alternatively, any substitutable nitrogen of a bridged bicyclic group is optionally substituted. The term “alkyl” refers to a C₁₋₁₂ straight or branched saturated aliphatic group. In certain instances, alkyl refers to a C₁₋₈ straight or branched saturated aliphatic group or a C₁₋₆ straight or branched saturated aliphatic group. The term “lower alkyl” refers to a C₁₋₄ straight or branched alkyl group.

Exemplary lower alkyl groups are methyl, ethyl, propyl, isopropyl (also referred to interchangeably herein as 2-propyl, iPr, ⁱPr and i-Pr), butyl, isobutyl (also referred to interchangeably herein as 2-butyl, iBu, ⁱBu and i-Bu) and tert-butyl (also referred to interchangeably herein as 2-methyl-2-butyl, tBu, ^{t}Bu and t-Bu).

The term “alkenyl” refers to a C₂₋₁₂ straight or branched partially unsaturated aliphatic group comprising at least one unsaturated carbon carbon double bond. In certain instances, alkenyl refers to a C₂₋₈ or a C₁₋₆ straight or branched partially unsaturated aliphatic group comprising at least one unsaturated carbon carbon double bond. The term “lower alkenyl” refers to a C₂₋₄ straight or branched partially unsaturated aliphatic group comprising at least one unsaturated carbon carbon double bond. Alkenyl groups include both cis (Z) and trans (E) regioisomers. Exemplary lower alkenyl groups are vinyl, allyl, 2-propenyl, and butenyl isomers (—CH₂CH₂CH═CH₂, —CH₂CH═CHCH₃ and —CH═CHCH₂CH₃).

The term “alkynyl” refers to a C₂₋₁₂ straight or branched partially unsaturated aliphatic group comprising at least one unsaturated carbon carbon triple bond. In certain instances, alkynyl refers to a C₂₋₈ or a C₁₋₆ straight or branched partially unsaturated aliphatic group comprising at least one unsaturated carbon carbon triple bond. The term “lower alkynyl” refers to a C₂₋₄ straight or branched partially unsaturated aliphatic group comprising at least one unsaturated carbon carbon triple bond. Exemplary lower alkynyl groups are ethynyl, 1-propynyl, 2-propynyl, 1-butynyl, 2-butynyl, and 3-butynyl.

The term “haloalkyl” refers to a straight or branched alkyl group that is substituted with one or more halogen atoms. The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, 9 or 10 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, pyrazinyl, pyrazolyl, pyridazinyl, pyridyl, pyrimidinyl, triazinyl, thienyl, furanyl, pyrrolyl, imidazolyl, pyrazolyl, triazolyl (i.e., 1,2,3triazolyl), 1,2,4triazolyl, 1,2,5triazolyl, 1,3,4triazolyl, tetrazolyl, oxazolyl, isoxazolyl, oxadiazolyl, 1,2,3oxadiazolyl, 1,2,4oxadiazolyl, 1,2,5-oxadiazolyl, 1,3,4oxadiazolyl, thiazolyl, isothiazolyl, thiadiazolyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, indolizinyl, isoindolin-1-only, 1,2-dihydro-3H-pyrrolo[3,4-c]pyridin-3-onyl, 2,3-dihydro-1H-pyrrolo[3,4-c]pyridin-1-only, imidazo[1,2-a]pyridyl, imidazo[1,5-a]pyridyl, pyrazolo[1,5-a]pyridyl, pyrrolo[1,2-b]pyridazinyl, pyrrolo[1,2-a]pyrimidinyl, imidazo[1,2-b]pyridazinyl, imidazo[1,2-a]pyrimidinyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, oxetanyl, azetidinyl, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; (CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —N(R^{∘})C(NR^{∘})N(R^{∘})₂; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —(CH₂)₀₋₄P(O)₂R^{∘}; —(CH₂)₀₋₄P(O)R^{∘}₂; —(CH₂)₀₋₄OP(O)R^{∘}₂; —(CH₂)₀₋₄OP(O)(OR^{∘})₂; —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 3-12-membered saturated, partially unsaturated, monocyclic, bicyclic, bridged bicyclic, or spirocyclic, or aryl mono- or bicyclic ring having 0-4 heteroatoms independently selected from nitrogen, oxygen, or sulfur, which may be substituted as defined below, or, notwithstanding the definition above, two independent occurrences of R^{∘}, taken together with their intervening atom(s), form a 3-12-membered saturated, partially unsaturated, monocyclic, bicyclic, bridged bicyclic, or spirocyclic, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{●} include halogen, —R^{●}, (haloR^{●}), OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, S(O)₂R^{†}, S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, (haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, Z and E conformational isomers and Rₐ (or M) and Sₐ (or P) atropisomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ³C- or ¹⁴C-enriched carbon are within the scope of this invention. Such compounds are useful, for example, as analytical tools, as probes in biological assays, or as therapeutic agents in accordance with the present invention.

The structures as drawn represent absolute configurations unless indicated otherwise. The invention contemplates individual enantiomers and diastereomers, enantiomerically enriched enantiomers and diastereomers, and racemic mixtures of enantiomers and diastereomers.

As used herein, a “SIK2 inhibitor” is a molecule that reduces, inhibits, or otherwise diminishes one or more of the biological activities of SIK2 (e.g., protein phosphorylation). Inhibition using the SIK2 inhibitor does not necessarily indicate a total elimination of the SIK2 activity. Instead, the activity could decrease by a statistically significant amount including, for example, a decrease of at least about 5%, 10%, 15%, 20%, 25%, 30%, 35%, 40%, 45%, 50%, 55%, 60%, 65%, 70%, 75%, 80%, 85%, 95% or 100% of the activity of SIK2 compared to an appropriate control.

A compound of the present invention may be tethered to a detectable moiety. It will be appreciated that such compounds are useful as imaging agents. One of ordinary skill in the art will recognize that a detectable moiety may be attached to a provided compound via a suitable substituent. As used herein, the term “suitable substituent” refers to a moiety that is capable of covalent attachment to a detectable moiety. Such moieties are well known to one of ordinary skill in the art and include groups containing, e.g., a carboxylate moiety, an amino moiety, a thiol moiety, or a hydroxyl moiety, to name but a few. It will be appreciated that such moieties may be directly attached to a provided compound or via a tethering group, such as a bivalent saturated or unsaturated hydrocarbon chain. In some embodiments, such moieties may be attached via click chemistry. In some embodiments, such moieties may be attached via a 1,3-cycloaddition of an azide with an alkyne, optionally in the presence of a copper catalyst. Methods of using click chemistry are known in the art and include those described by Rostovtsev et al., Angew. Chem. Int. Ed. 2002, 41:2596-99 and Sun et al., Bioconjugate Chem., 2006, 17:52-57.

As used herein, the term “detectable moiety” is used interchangeably with the term “label” and relates to any moiety capable of being detected, e.g., primary labels and secondary labels. Primary labels, such as radioisotopes (e.g., tritium, ³²P, ³³P, ³⁵S, or ¹⁴C), mass-tags, and fluorescent labels are signal generating reporter groups which can be detected without further modifications. Detectable moieties also include luminescent and phosphorescent groups.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label”, “fluorescent dye”, and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The terms “measurable affinity” and “measurably inhibit,” as used herein, means a measurable change in a SIK2 protein kinase activity between a sample comprising a compound of the present invention, or composition thereof, and a SIK2 protein kinase, and an equivalent sample comprising a SIK2 protein kinase, in the absence of said compound, or composition thereof.

### 3. Description of Exemplary Embodiments

As described above, in certain embodiments, the present invention provides a compound of formula I:

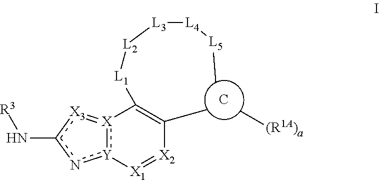

or a pharmaceutically acceptable salt thereof, wherein:
X is N or C;
Y is N or C, where when one of X and Y is N and the other of X and Y is C;
Each [symbol] is a single or double bond;
L₁ is selected from a bond, —O—, —S—, —S(O)₂—, —NR¹—, —C(O)—, —C(O)NR¹—, —NR¹C(O)—, —NR¹C(O)NR¹—, and —[C(R^{1a})(R^{1b})]₁₋₂—;
L₂ is selected from a bond, a substituted or unsubstituted alkenylene, —O—, —S—, —S(O)₂—, —N═S(O)(CH₃)—, —S(O)(CH₃)═N—, —S(O)₂NR¹—, —NR¹S(O)₂—, —NR¹—, —C(O)—, —C(O)NR¹—, —NR¹C(O)—, —NR¹C(O)NR¹—, an optionally substituted C₃-C₆ cycloalkyl, an optionally substituted C₃-C₆ heterocycloalkyl (having 1-4 heteroatoms independently selected from nitrogen, oxygen, and sulfur), an optionally substituted 5-6 membered monocyclic heteroaryl ring having 1-3 heteroatoms independently selected from nitrogen, oxygen, and sulfur; and —[C(R^{2a})(R^{2b})]₁₋₂—;
L₃ is selected from a bond, a substituted or unsubstituted alkenylene, —O—, —S—, —S(O)₂—, —N═S(O)(CH₃)—, —S(O)(CH₃)═N—, —S(O)₂NR¹—, —NR¹S(O)₂—, —NR¹—, —C(O)—, —C(O)NR¹—, —NR¹C(O)—, —NR¹C(O)NR¹—, an optionally substituted C₃-C₆ cycloalkyl, an optionally substituted C₃-C₆ heterocycloalkyl (having 1-4 heteroatoms independently selected from nitrogen, oxygen, and sulfur), an optionally substituted 5-6 membered monocyclic heteroaryl ring having 1-3 heteroatoms independently selected from nitrogen, oxygen, and sulfur; and —[C(R^{3a})(R^{3b})]₁₋₂—;
L₄ is selected from a bond, a substituted or unsubstituted alkenylene, —O—, —S—, —S(O)₂—, —N═S(O)(CH₃)—, —S(O)(CH₃)═N—, —S(O)₂NR¹—, —NR¹S(O)₂—, —NR¹—, —C(O)—, —C(O)NR¹—, —NR¹C(O)—, —NR¹C(O)NR¹—, an optionally substituted C₃-C₆ cycloalkyl, an optionally substituted C₃-C₆ heterocycloalkyl (having 1-4 heteroatoms independently selected from nitrogen, oxygen, and sulfur), an optionally substituted 5-6 membered monocyclic heteroaryl ring having 1-3 heteroatoms independently selected from nitrogen, oxygen, and sulfur; and —[C(R^{4a})(R^{4b})]₁₋₂—;
L₅ is selected from a bond, —O—, —S—, —S(O)₂—, —NR¹—, —C(O)—, —C(O)NR¹—, —NR¹C(O)—, —NR¹C(O)NR₁—, and —[C(R^{5a})(R^{5b})]₁₋₂—;
   Each R¹ is independently selected from the group consisting of hydrogen, C₁₋₃ haloalkyl, C₁₋₃ hydroxyalkyl, C₀₋₃ alkyl-C₃-C₆ cycloalkyl, C₀₋₃ alkyl-C₃-C₆ heterocycloalkyl (having 1-4 heteroatoms independently selected from nitrogen, oxygen, and sulfur), and C₁₋₃ alkyl; wherein C₀₋₃ alkyl-C₃-C₆ cycloalkyl, C₀₋₃ alkyl-C₃-C₆ heterocycloalkyl and C₁₋₃ alkyl are substituted with y instances of R^{B};
   each R^{B} is independently halogen, C₁₋₃ alkyl, C₁₋₃ hydroxyalkyl, —CN, oxo, —OR, or —NR₂;
   each y is independently 0, 1, 2, or 3;
R^{1a}, R^{1b}, R^{2a}, R^{2a}, R^{3a}, R^{3b}, R^{4a}, R^{4b}, R^{5a}, and R^{5b} are independently selected from the group consisting of hydrogen, halogen, hydroxyl, N(R¹)₂, C₁₋₃ alkyl, —CN, C₁₋₃ alkoxy, C₀₋₃ alkyl-C₃-C₆ cycloalkyl, C₀₋₃ alkyl-C₃-C₆ heterocycloalkyl, and C₁₋₃ hydroxyalkyl, and C₁₋₃ haloalkyl, wherein C₀₋₃ alkyl-C₃-C₆ cycloalkyl, C₀₋₃ alkyl-C₃-C₆ heterocycloalkyl and C₁₋₃ alkyl are substituted with y instances of R^{B}; or
R^{1a} and R^{1b}, R^{2a} and R^{2b}, R^{3a} and R^{3b}, R^{4a} and R^{4b}, or R^{5a} and R^{5b}, and the carbon atom to which they are attached may combine to form:
   a) a 3-7 membered saturated or partially unsaturated bivalent monocyclic carbocyclylene or 4-7 membered saturated or partially unsaturated bivalent heterocyclylene ring (having 1-4 heteroatoms independently selected from nitrogen, oxygen, and sulfur, and 0 or 1 nitrogen atoms in addition to the 1-4 heteroatoms)—substituted with y instances of R^{B}, or
   b) a 4-12 membered saturated or partially unsaturated bivalent bicyclic ring system that is fused, bridged, or spirocyclic selected from carbocyclylene or heterocyclylene (having 1-4 heteroatoms independently selected from nitrogen, oxygen, and sulfur)—substituted with y instances of R^{B}, or
R^{1a} and R^{2a}, R^{1a} and R^{3a}, R^{2a} and R^{3a}, R^{2a} and R^{4a}, R^{3a} and R^{4a}, R^{3a} and R^{5a}, or R^{4a} and R^{5a}, and the atoms to which they are connected may combine to form:
   a) a 3-7 membered saturated or partially unsaturated bivalent monocyclic carbocyclylene or a 4-7 membered saturated or partially unsaturated bivalent heterocyclylene ring (having 1-4 heteroatoms independently selected from nitrogen, oxygen, and sulfur, and 0 or 1 nitrogen atoms in addition to the 1-4 heteroatoms)—substituted with y instances of R^{B}, or
   b) a 4-12 membered saturated or partially unsaturated bivalent bicyclic ring system that is fused, bridged, or spirocyclic selected from carbocyclylene or heterocyclylene (having 1-4 heteroatoms independently selected from nitrogen, oxygen, and sulfur)—substituted with y instances of R^{B};
X₁ and X₂ are each selected from N and CR² provided when Y is N, X₁ is CR²;
each R² is independently selected from hydrogen, OH, C₁₋₃ alkyl, C₁₋₃ alkoxy, and halogen;
X₃ is selected from O, S, N, NR⁷, and CR², provided when X₃ is selected from O, S, and NR⁷, X and Y are C;
R⁷ is hydrogen or C₁₋₃ alkyl;
Ring C is a 5-6 membered monocyclic heteroaryl ring having 1-3 heteroatoms independently selected from nitrogen, oxygen, and sulfur; an 8-10 membered bicyclic heteroaryl ring having 1-4 heteroatoms independently selected from nitrogen, oxygen, and sulfur; or a 7-12 membered saturated or partially unsaturated bicyclic heterocyclyl ring having 1-4 heteroatoms independently selected from nitrogen, oxygen, and sulfur;
each R^{1A} is independently halogen, —CN, oxo, —OR, —SR, —NR₂, —SO₂R, —SO₂NR₂, —S(O)R, —S(O)NR₂, —C(O)R, —CO₂R, —C(O)NR₂, —C(O)N(R)OR, —OC(O)R, —OC(O)NR₂, —N(R)CO₂R, —N(R)C(O)R, —N(R)C(O)NR₂, —N(R)C(NR)R, —N(R)C(NR)NR₂, —N(R)NR₂, —N(R)SO₂NR₂, —N(R)SO₂R, —N═S(O)R₂, —S(NR)(O)R, —N(R)S(O)R, —N(R)CN, —P(O)(OR)₂, —P(O)R₂, C₁-C₆ haloalkyl, C₁₋₃ hydroxyalkyl, C₃-C₆ halocycloalkyl, C₁-C₆ haloalkoxy, or an optionally substituted group selected from C₁₋₆ aliphatic; phenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; and a 5-6 membered monocyclic heteroaryl ring having 1-4 heteroatoms independently selected from nitrogen, oxygen, and sulfur;
a is 0, 1, 2, or 3;
R³ is —C(O)-L₆, —S(O)₂-L₆, an optionally substituted C₁₋₆ aliphatic, an optionally substituted C₁₋₆ aliphatic substituted with

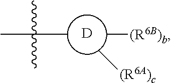

an optionally substituted 5-6 membered monocyclic heteroaromatic ring having 1-4 heteroatoms independently selected from nitrogen, oxygen, and sulfur, or

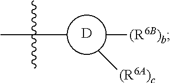

L₆ is selected from the group consisting of

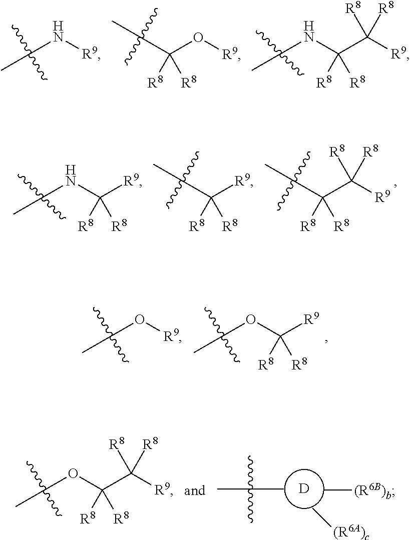

Ring D is a 3-8 membered saturated or partially unsaturated monocyclic carbocyclic, a 3-8 membered saturated or partially unsaturated monocyclic heterocyclic ring having 1-2 heteroatoms independently selected from nitrogen, oxygen, and sulfur, a 5-12 membered saturated or partially unsaturated bicyclic heterocyclic ring that is optionally bridged or spirocyclic having 1-4 heteroatoms independently selected from nitrogen, oxygen, and sulfur, or a 7-14 membered saturated or partially unsaturated tricyclic heterocyclic ring that is optionally bridged or spirocyclic having 1-4 heteroatoms independently selected from nitrogen, oxygen, and sulfur;
Each R⁸ is independently selected from hydrogen, halogen, hydroxyl, —CN, C₁₋₄ alkoxy, a 3-8 membered saturated or partially unsaturated monocyclic carbocyclic, C₁-C₆haloalkyl, and C₁₋₄ alkyl; or two R⁸ groups and the atom to which they are attached form oxo, a 3-8 membered saturated or partially unsaturated monocyclic carbocyclic ring or a 3-8 membered saturated or partially unsaturated monocyclic heterocyclic ring having 1-4 heteroatoms independently selected from nitrogen, oxygen, and sulfur;
R⁹ is selected from hydrogen, C₁-C₆ alkyl, C₁-C₆ haloalkyl, C₁₋₃ hydroxyalkyl, C₁-C₆ alkoxy, C₁-C₆ haloalkoxy, and an optionally substituted C₁₋₆ aliphatic; or
R⁹ is a cyclic group or an optionally substituted C₁₋₆ aliphatic substituted with a cyclic group,
   wherein the cyclic group is selected from:
      phenyl;
      a 3-8 membered saturated or partially unsaturated monocyclic carbocyclic ring;
      a 3-8 membered saturated or partially unsaturated monocyclic heterocyclic ring having 1-2 heteroatoms independently selected from nitrogen, oxygen, and sulfur;
      a 5-6 membered monocyclic heteroaromatic ring having 1-4 heteroatoms independently selected from nitrogen, oxygen, and sulfur;
      an 8-10 membered bicyclic heteroaromatic ring having 1-5 heteroatoms independently selected from nitrogen, oxygen, and sulfur;
      a 7-12 membered saturated or partially unsaturated bicyclic heterocyclic ring having 1-4 heteroatoms independently selected from nitrogen, oxygen, and sulfur;
      a 5-12 membered saturated or partially unsaturated bicyclic carbocyclic ring that is optionally bridged or spirocyclic; and
      a 5-12 membered saturated or partially unsaturated bicyclic heterocyclic ring that is optionally bridged or spirocyclic having 1-4 heteroatoms independently selected from nitrogen, oxygen, and sulfur,
   wherein the cyclic group is substituted with y instances of R^{A};
each R^{6A} is independently a C₁₋₆ aliphatic-cyclic or cyclic group, wherein the cyclic group is selected from:
   a 5-6 membered monocyclic heteroaromatic ring having 1-4 heteroatoms independently selected from nitrogen, oxygen, and sulfur;
   an 8-10 membered bicyclic heteroaromatic ring having 1-5 heteroatoms independently selected from nitrogen, oxygen, and sulfur;
   a 7-12 membered saturated or partially unsaturated bicyclic heterocyclic ring having 1-4 heteroatoms independently selected from nitrogen, oxygen, and sulfur;
   a 5-12 membered saturated or partially unsaturated bicyclic carbocyclic ring that is optionally bridged or spirocyclic; and
   a 5-12 membered saturated or partially unsaturated bicyclic heterocyclic ring that is optionally bridged or spirocyclic having 1-4 heteroatoms independently selected from nitrogen, oxygen, and sulfur,
   wherein R^{6A} is substituted with y instances of R^{A};
each R^{6B} is independently halogen, —CN, oxo, —OR, —SR, —NR₂, —SO₂R, —SO₂NR₂, —S(O)R, —S(O)NR₂, —C(O)R, —CO₂R, —C(O)NR₂, —C(O)N(R)OR, —OC(O)R, —OC(O)NR₂, —N(R)CO₂R, —N(R)C(O)R, —N(R)C(O)NR₂, —N(R)C(NR)R, —N(R)C(NR)NR₂, —N(R)NR₂, —N(R)SO₂NR₂, —N(R)SO₂R, —N═S(O)R₂, —S(NR)(O)R, —N(R)S(O)R, —N(R)CN, —P(O)(OR)₂, —P(O)R₂, or an optionally substituted group selected from C₁₋₆ aliphatic; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; and a 5-6 membered monocyclic heteroaryl ring having 1-4 heteroatoms independently selected from nitrogen, oxygen, and sulfur;
b is 0, 1, 2, or 3;
c is 0, 1, 2, or 3;
each R^{A} is independently selected from deuterium, a C₁₋₆ aliphatic group substituted with 0, 1, 2, or 3 groups independently selected from halogen, deuterium, —CN, —NR₂, and —OR; a 5-6 membered monocyclic heteroaromatic ring having 1-4 heteroatoms independently selected from nitrogen, oxygen, and sulfur substituted with 0, 1, 2, or 3 groups independently selected from halogen, C₁₋₃ alkyl, C₁₋₃ haloalkyl, —CN, —NR₂, and —OR; a 3-8 membered saturated or partially unsaturated monocyclic heterocyclic ring having 1-2 heteroatoms independently selected from nitrogen, oxygen, and sulfur substituted with 0, 1, 2, or 3 groups independently selected from halogen, C₁₋₃ alkyl, C₁₋₃ haloalkyl, —CN, —NR₂, and —OR; a 3-8 membered saturated or partially unsaturated monocyclic carbocyclic ring substituted with 0, 1, 2, or 3 groups independently selected from halogen, C₁₋₃ alkyl, C₁₋₃ haloalkyl, —CN, —NR₂, and —OR; a 4-12 membered saturated or partially unsaturated bicyclic ring system that is fused, bridged, or spirocyclic selected from carbocyclyl and heterocyclyl (having 1-4 heteroatoms independently selected from nitrogen, oxygen, and sulfur), wherein said carbocyclyl or heterocyclyl is substituted with 0, 1, 2, or 3 groups independently selected C₁₋₃ alkyl, C₁₋₃ alkoxy, C₁₋₃ haloalkyl, C₁₋₃ hydroxyalkyl, halogen, —CN, —OR, and —NR₂; halogen; —CN; —NO₂; —OR; —SR; —NR₂; —S(O)₂R; —S(O)₂NR₂; —S(O)R; —S(O)NR₂; —C(O)R; —C(O)OR; —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —S(NR)(O)R; —N(R)S(O)₂NR₂; and —N(R)S(O)₂R; or
   two R^{A} on the same atom together form oxo or a cyclic group selected from:
      3-8 membered saturated or partially unsaturated monocyclic carbocyclic ring; and
      a 3-8 membered saturated or partially unsaturated monocyclic heterocyclic ring having 1-2 heteroatoms independently selected from nitrogen, oxygen, and sulfur;
   wherein the cyclic group formed by two R^{A} on the same atom is substituted with 0, 1, 2, or 3 groups independently selected from a C₁₋₆ aliphatic group substituted with 0, 1, 2, or 3 groups independently selected from halogen, —CN, —NR₂, and —OR; halogen; —CN; —NO₂; —OR; —SR; —NR₂; —S(O)₂R; —S(O)₂NR₂; —S(O)R; —S(O)NR₂; —C(O)R; —C(O)OR; —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —N(R)S(O)₂NR₂; and —N(R)S(O)₂R; or
   two R^{A} on adjacent atoms form a cyclic group selected from:
      3-8 membered saturated or partially unsaturated monocyclic carbocyclic ring;
      a 3-8 membered saturated or partially unsaturated monocyclic heterocyclic ring having 1-2 heteroatoms independently selected from nitrogen, oxygen, and sulfur;
      phenyl; and
      a 5-6 membered heteroaryl ring having 1-4 heteroatoms independently selected from nitrogen, oxygen, and sulfur,
   wherein the cyclic group formed by two R^{A} on adjacent atoms is substituted with 0, 1, 2, or 3 groups independently selected from a C₁₋₆ aliphatic group substituted with 0, 1, 2, or 3 groups independently selected from halogen, —CN, —NR₂, and —OR; halogen; —CN; —NO₂; —OR; —SR; —NR₂; —S(O)₂R; —S(O)₂NR₂; —S(O)R; —S(O)NR₂; —C(O)R; —C(O)OR; —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —N(R)S(O)₂NR₂; and —N(R)S(O)₂R;
each R is independently hydrogen, —C(O)N(CH₃)₂, —C(O)₂CH₃, —C(O)₂C(CH₃)₃, —C(O)₂CH(CH₃)₂, —S(O)₂CH₃, an optionally substituted C₁₋₆ aliphatic group, an optionally substituted cyclic group, or an optionally substituted C₁₋₆ alkyl-cyclic group, wherein the cyclic group is selected from:
   an optionally substituted phenyl;
   an optionally substituted 3-7 membered saturated or partially unsaturated carbocyclic ring;
   an optionally substituted 3-7 membered saturated or partially unsaturated heterocyclic ring having 1-2 heteroatoms independently selected from nitrogen, oxygen, and sulfur;
   an optionally substituted 5-6 membered heteroaryl ring having 1-4 heteroatoms independently selected from nitrogen, oxygen, and sulfur;
   an optionally substituted 8-10 membered bicyclic heteroaryl ring having 1-4 heteroatoms independently selected from nitrogen, oxygen, and sulfur;
   an optionally substituted 7-12 membered saturated or partially unsaturated bicyclic heterocyclic ring having 1-4 heteroatoms independently selected from nitrogen, oxygen, and sulfur;
   an optionally substituted 5-8 membered saturated or partially unsaturated bridged bicyclic ring having 0-3 heteroatoms independently selected from nitrogen, oxygen, and sulfur;
   an optionally substituted 6-10 membered saturated or partially unsaturated spirocyclic ring having 0-3 heteroatoms independently selected from nitrogen, oxygen, and sulfur; and
   an optionally substituted 6-11 membered saturated or partially unsaturated bicyclic ring having 0-2 heteroatoms independently selected from nitrogen, oxygen, and sulfur; and/or
   two R groups on the same atom are taken together with the same atom to form a cyclic group selected from:
      an optionally substituted 4-7 membered saturated or partially unsaturated carbocyclyl;
      an optionally substituted 4-7 membered saturated or partially unsaturated heterocyclyl having 1-3 heteroatoms independently selected from nitrogen, oxygen, and sulfur; and
      an optionally substituted 5-6 membered heteroaryl ring having 1-3 heteroatoms independently selected from nitrogen, oxygen, and sulfur, wherein the optionally substituted R moiety is substituted with 0, 1, 2, 3, or 4 R^{Sub};
   each R^{Sub} is independently selected from C₁₋₆ aliphatic group substituted with 0, 1, 2, or 3 groups independently selected from halogen, —CN, —N(R^{N})₂, and —O R^{N}; halogen; —CN; —NO₂; —OR^{N}; —SR^{N}; —N(R^{N})₂; —S(O)₂R^{N}; —S(O)₂N(R^{N})₂; —S(O)R^{N}; —S(O)N(R^{N})R₂; —C(O)R^{N}; —C(O)OR^{N}; —C(O)N(R^{N})₂; —C(O)N(R^{N})OR^{N}; —OC(O)R^{N}; —OC(O)N(R^{N})₂; —N(R^{N})C(O)OR^{N}; —N(R^{N})C(O)R^{N}; —N(R^{N})C(O)N(R^{N})₂; —N(R^{N})C(NRN)N(R^{N})₂; —N(R^{N})S(O)₂N(R^{N})₂; and —N(R^{N})S(O)₂R^{N}; and each R^{N} is independently selected from H and C₁₋₃ alkyl.

In some embodiments, the compound of formula I is a compound of formula I-a, I-b, or I-c:

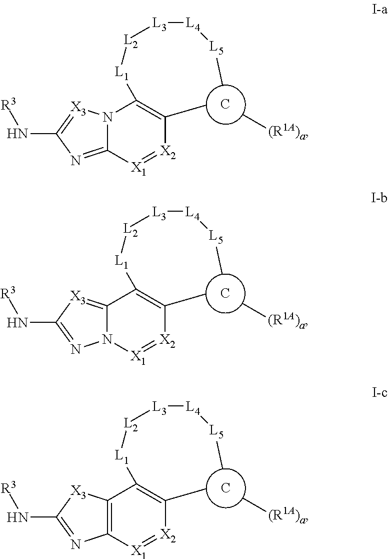

or a pharmaceutically acceptable salt thereof, wherein each of X₁, X₂, X₃, R^{1A}, R³, L₁, L₂, L₃, L₄, L₅, a, and Ring C are as defined herein, both singly and in combination, and Ring C is a 5-membered monocyclic heteroaryl having 1-3 heteroatoms independently selected from nitrogen, oxygen, and sulfur.

In some embodiments, the compound of formula I-a, I-b, or I-c:

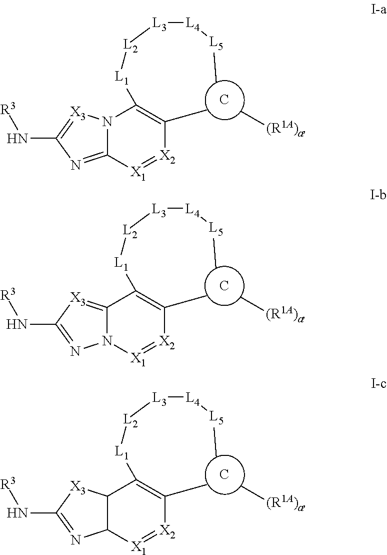

or a pharmaceutically acceptable salt thereof, wherein each of X₁, X₂, X₃, R^{1A}, R³, L₁, L₂, L₃, L₄, L₅, a, as defined herein, both singly and in combination, and Ring C is a 6-membered monocyclic heteroaryl having 1-3 heteroatoms independently selected from nitrogen, oxygen, and sulfur.

In some embodiments, the compound of formula I-a, I-b, or I-c:

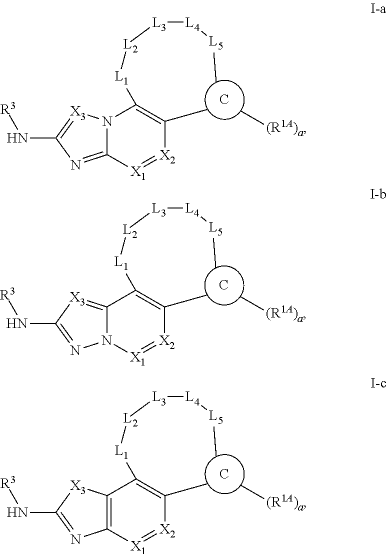

or a pharmaceutically acceptable salt thereof, wherein each of X₁, X₂, X₃, R^{1A}, R³, L₁, L₂, L₃, L₄, L₅, a, as defined herein, both singly and in combination, and Ring C is a 7-12 membered saturated or partially unsaturated bicyclic heterocyclyl ring having 1-4 heteroatoms independently selected from nitrogen, oxygen, and sulfur.

In some embodiments, the compound of formula I-a, I-b, or I-c:

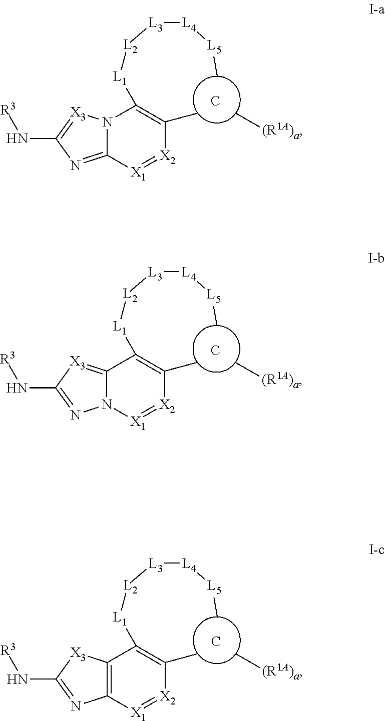

or a pharmaceutically acceptable salt thereof, wherein each of X₁, X₂, X₃, R^{1A}, R³, L₁, L₂, L₃, L₄, L₅, a, as defined herein, both singly and in combination, and Ring C is an 8-10 membered bicyclic heteroaryl ring having 1-4 heteroatoms independently selected from nitrogen, oxygen, and sulfur.

As defined generally above, X is N or C.

In some embodiments, X is N. In some embodiments, X is C.

As defined generally above, Y is N or C.

In some embodiments, X is N. In some embodiments, X is C.

In certain embodiments, one of X and Y is N and the other of X and Y is C;

In some embodiments, X and Y are as depicted in the compounds of Table 1, below.

As defined generally above, [symbol] is a single or double bond.

In some embodiments, [symbol] is a single bond. In some embodiments, [symbol] is a double bond.

In some embodiments, [symbol] is as depicted in the compounds of Table 1, below.

As defined generally above, X₁ and X₂ are independently selected from N and CR² provided that none are N or only one of X₁ and X₂ may be N.

In some embodiments, X₁ is N. In some embodiments, X₁ is CR².

In some embodiments, X₂ is N. In some embodiments, X₂ is CR².

In some embodiments, X₁, and X₂ are CR². In some embodiments, X₁ is N, and X₂ and X₃ are CR². In some embodiments, X₂ is N, and X₁ and X₃ are CR². In some embodiments, X₃ is N, and X₁ and X₂ are CR².

In some embodiments, X₁ is as depicted in the compounds of Table 1, below.

In some embodiments, X₂ is as depicted in the compounds of Table 1, below

In some embodiments, X₃ is selected from O, N, S, NR⁷ and CR²;

In some embodiments, X₃ is O.

In some embodiments, X₃ is S.

In some embodiments, X₃ is NR⁷.

In some embodiments, X₃ is N.

In some embodiments, X₃ is CR².

In some embodiments, X₃ is as depicted in the compounds of Table 1, below.

As defined generally above, each R² is independently selected from hydrogen, OH, C₁₋₃ alkyl, C₁₋₃ alkoxy, and halogen.

In some embodiments, R² is hydrogen. In some embodiments, R² is OH. In some embodiments, R² is C₁₋₃ alkyl. In some embodiments, R² is C₁₋₃ alkoxy. In some embodiments, R² is halogen.

In some embodiments, R² is hydrogen, F, Cl, CH₃, OCH₃, or CH₂CH₃.

In some embodiments, R² is F.

In some embodiments, R² is Cl.

In some embodiments, R² is CH₃.

In some embodiments, R² is OCH₃.

In some embodiments, R² is CH₂CH₃.

In some embodiments, R² is CH(CH₃)₂.

In some embodiments, R² is as depicted in the compounds of Table 1, below.

As defined generally above, R⁷ is hydrogen or C₁₋₄ alkyl.

In some embodiments, R⁷ is hydrogen. In some embodiments, R⁷ is C₁₋₄ alkyl.

In some embodiments, R⁷ is methyl.

In some embodiments, R⁷ is as depicted in the compounds of Table 1, below.

As defined generally above, Ring C is a 5-6 membered monocyclic heteroaryl ring having 1-3 heteroatoms independently selected from nitrogen, oxygen, and sulfur; an 8-10 membered bicyclic heteroaryl ring having 1-4 heteroatoms independently selected from nitrogen, oxygen, and sulfur; or a 7-12 membered saturated or partially unsaturated bicyclic heterocyclyl ring having 1-4 heteroatoms independently selected from nitrogen, oxygen, and sulfur.

In some embodiments, Ring C is a 5-6 membered monocyclic heteroaryl ring having 1-3 heteroatoms independently selected from nitrogen, oxygen, and sulfur. In some embodiments, Ring C is 8-10 membered bicyclic heteroaryl ring having 1-4 heteroatoms independently selected from nitrogen, oxygen, and sulfur. In some embodiments, Ring C is a 7-12 membered saturated or partially unsaturated bicyclic heterocyclyl ring having 1-4 heteroatoms independently selected from nitrogen, oxygen, and sulfur.

In some embodiments, Ring C is a 5-6 membered monocyclic heteroaryl ring having 1-3 heteroatoms independently selected from nitrogen, oxygen, and sulfur, a 9-membered bicyclic heteroaryl ring having 1-4 heteroatoms independently selected from nitrogen, oxygen, and sulfur, or a 9-membered partially unsaturated bicyclic heterocyclyl ring having 1-4 heteroatoms independently selected from nitrogen, oxygen, and sulfur.

In some embodiments, Ring C is a 5-membered monocyclic heteroaryl ring having 1-3 heteroatoms independently selected from nitrogen, oxygen, and sulfur.

In some embodiments, Ring C is

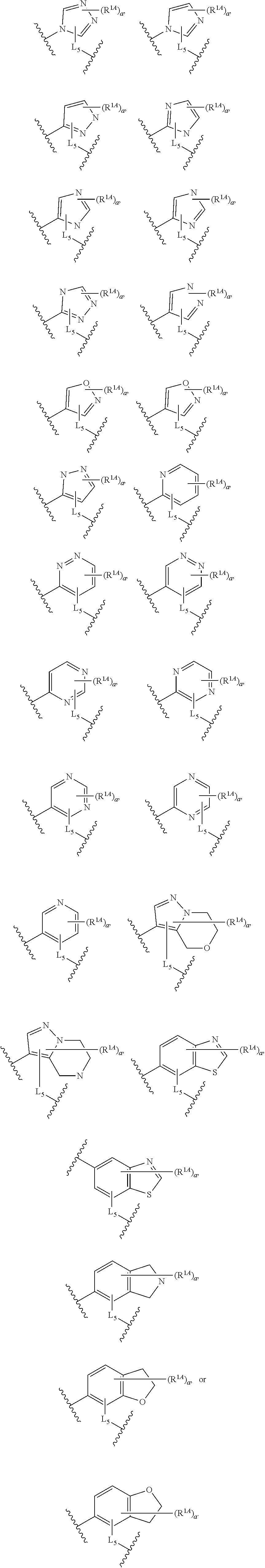

In some embodiments, Ring C is

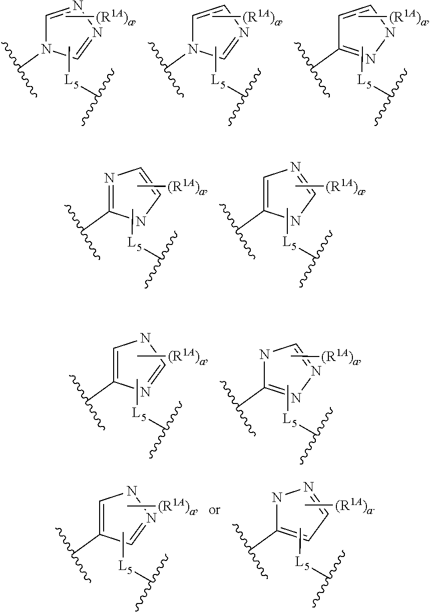

In some embodiments, Ring C is as depicted in the compounds of Table 1, below.

As defined generally above, Ring D is a 3-8 membered saturated or partially unsaturated monocyclic carbocyclic, a 3-8 membered saturated or partially unsaturated monocyclic heterocyclic ring having 1-2 heteroatoms independently selected from nitrogen, oxygen, and sulfur, a 5-12 membered saturated or partially unsaturated bicyclic heterocyclic ring that is optionally bridged or spirocyclic having 1-4 heteroatoms independently selected from nitrogen, oxygen, and sulfur, or a 7-14 membered saturated or partially unsaturated tricyclic heterocyclic ring that is optionally bridged or spirocyclic having 1-4 heteroatoms independently selected from nitrogen, oxygen, and sulfur.

In some embodiments, Ring D is a 3-8 membered saturated or partially unsaturated monocyclic carbocyclic. In some embodiments, Ring D is 3-8 membered saturated or partially unsaturated monocyclic heterocyclic ring having 1-2 heteroatoms independently selected from nitrogen, oxygen, and sulfur. In some embodiments, Ring D is a 5-12 membered saturated or partially unsaturated bicyclic heterocyclic ring that is optionally bridged or spirocyclic having 1-4 heteroatoms independently selected from nitrogen, oxygen, and sulfur. In some embodiments, Ring D is a 7-14 membered saturated or partially unsaturated tricyclic heterocyclic ring that is optionally bridged or spirocyclic having 1-4 heteroatoms independently selected from nitrogen, oxygen, and sulfur.

In some embodiments, Ring D is a 3-4 membered saturated or partially unsaturated monocyclic carbocyclic.

In some instances, Ring D is

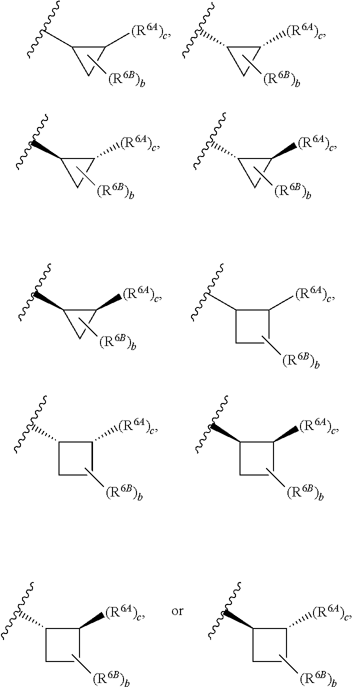

where b is 0, 1, 2, or 3, and c is 1.

In some embodiments, Ring D is as depicted in the compounds of Table 1, below.

As defined generally above, each R⁸ is independently selected from hydrogen, halogen, hydroxyl, —CN, C₁₋₄ alkoxy, a 3-8 membered saturated or partially unsaturated monocyclic carbocyclic, C₁-C₆ haloalkyl, and C₁₋₄ alkyl; or two R⁸ groups and the atom to which they are attached can form oxo, a 3-8 membered saturated or partially unsaturated monocyclic carbocyclic ring or a 3-8 membered saturated or partially unsaturated monocyclic heterocyclic ring having 1-4 heteroatoms independently selected from nitrogen, oxygen, and sulfur. In some embodiments, each R⁸ is independently selected from hydrogen, halogen, hydroxyl, —CN, C₁₋₄ alkoxy, C₁-C₆ haloalkyl, and C₁₋₄ alkyl; or two R⁸ groups and the atom to which they are attached can form oxo, a 3-8 membered saturated or partially unsaturated monocyclic carbocyclic ring or a 3-8 membered saturated or partially unsaturated monocyclic heterocyclic ring having 1-4 heteroatoms independently selected from nitrogen, oxygen, and sulfur.

In some embodiments, at least one R⁸ is hydrogen. In some embodiments, at least one R⁸ is halogen. In some embodiments, at least one R⁸ is hydroxyl. In some embodiments, at least one R⁸ is —CN. In some embodiments, at least one R⁸ is C₁₋₄ alkoxy. In some embodiments, at least one R⁸ is C₁₋₄ alkyl. In some embodiments, at least one R⁸ is a 3-8 membered saturated or partially unsaturated monocyclic carbocyclic. In some embodiments, two R⁸ groups and the atom to which they are attached form oxo. In some embodiments, at least one R⁸ is C₁-C₆ haloalkyl. In some embodiments, two R⁸ groups and the atom to which they are attached form a 3-8 membered saturated or partially unsaturated monocyclic carbocyclic ring. In some embodiments, two R⁸ groups and the atom to which they are attached form a 3-8 membered saturated or partially unsaturated monocyclic heterocyclic ring having 1-4 heteroatoms independently selected from nitrogen, oxygen, and sulfur.

As defined generally above, R⁹ is selected from hydrogen, C₁-C₆ alkyl, C₁-C₆ haloalkyl, C₁₋₃ hydroxyalkyl, C₁-C₆ alkoxy, C₁-C₆ haloalkoxy, and an optionally substituted C₁₋₆ aliphatic; or
R⁹ is a cyclic group or an optionally substituted C₁₋₆ aliphatic substituted with a cyclic group, wherein the cyclic group is selected from:
phenyl;
a 3-8 membered saturated or partially unsaturated monocyclic carbocyclic ring;
a 3-8 membered saturated or partially unsaturated monocyclic heterocyclic ring having 1-2 heteroatoms independently selected from nitrogen, oxygen, and sulfur;
a 5-6 membered monocyclic heteroaromatic ring having 1-4 heteroatoms independently selected from nitrogen, oxygen, and sulfur;
an 8-10 membered bicyclic heteroaromatic ring having 1-5 heteroatoms independently selected from nitrogen, oxygen, and sulfur;
a 7-12 membered saturated or partially unsaturated bicyclic heterocyclic ring having 1-4 heteroatoms independently selected from nitrogen, oxygen, and sulfur;
a 5-12 membered saturated or partially unsaturated bicyclic carbocyclic ring that is optionally bridged or spirocyclic; and
a 5-12 membered saturated or partially unsaturated bicyclic heterocyclic ring that is optionally bridged or spirocyclic having 1-4 heteroatoms independently selected from nitrogen, oxygen, and sulfur,
wherein the cyclic group is substituted with y instances of R^{A}.

In some embodiments, R⁹ is hydrogen. In some embodiments, R⁹ is C₁-C₆ alkyl. In some embodiments, R⁹ is C₁-C₆ haloalkyl. In some embodiments, R⁹ is C₁₋₃ hydroxyalkyl. In some embodiments, R⁹ is C₁-C₆ alkoxy. In some embodiments, R⁹ is C₁-C₆ haloalkoxy. In some embodiments, R⁹ is an optionally substituted C₁₋₆ aliphatic. In some embodiments, R⁹ is a cyclic group. In some embodiments, R⁹ is an optionally substituted C₁₋₆ aliphatic substituted with a cyclic group. In some embodiments, R⁹ is phenyl. In some embodiments, the cyclic group of R⁹ is a 3-8 membered saturated or partially unsaturated monocyclic carbocyclic ring. In some embodiments, the cyclic group of R⁹ is a 3-8 membered saturated or partially unsaturated monocyclic heterocyclic ring having 1-2 heteroatoms independently selected from nitrogen, oxygen, and sulfur. In some embodiments, the cyclic group of R⁹ is a 5-6 membered monocyclic heteroaromatic ring having 1-4 heteroatoms independently selected from nitrogen, oxygen, and sulfur. In some embodiments, the cyclic group of R⁹ is an 8-10 membered bicyclic heteroaromatic ring having 1-5 heteroatoms independently selected from nitrogen, oxygen, and sulfur. In some embodiments, the cyclic group of R⁹ is a 7-12 membered saturated or partially unsaturated bicyclic heterocyclic ring having 1-4 heteroatoms independently selected from nitrogen, oxygen, and sulfur. In some embodiments, the cyclic group of R⁹ is a 5-12 membered saturated or partially unsaturated bicyclic carbocyclic ring that is optionally bridged or spirocyclic. In some embodiments, the cyclic group of R⁹ is a 5-12 membered saturated or partially unsaturated bicyclic heterocyclic ring that is optionally bridged or spirocyclic having 1-4 heteroatoms independently selected from nitrogen, oxygen, and sulfur. In some embodiments, the cyclic group of R⁹ is substituted with y instances of R^{A}.

As defined generally above, each R^{6A} is independently a C₁₋₆ aliphatic-cyclic or cyclic group, wherein the cyclic group is selected from:
a 3-8 membered saturated or partially unsaturated monocyclic carbocyclic ring,
phenyl;
a 3-8 membered saturated or partially unsaturated monocyclic heterocyclic ring having 1-2 heteroatoms independently selected from nitrogen, oxygen, and sulfur;
a 5-6 membered monocyclic heteroaromatic ring having 1-4 heteroatoms independently selected from nitrogen, oxygen, and sulfur;
an 8-10 membered bicyclic heteroaromatic ring having 1-5 heteroatoms independently selected from nitrogen, oxygen, and sulfur;
a 7-12 membered saturated or partially unsaturated bicyclic heterocyclic ring having 1-4 heteroatoms independently selected from nitrogen, oxygen, and sulfur;
a 5-12 membered saturated or partially unsaturated bicyclic carbocyclic ring that is optionally bridged or spirocyclic; and
a 5-12 membered saturated or partially unsaturated bicyclic heterocyclic ring that is optionally bridged or spirocyclic having 1-4 heteroatoms independently selected from nitrogen, oxygen, and sulfur.

In some embodiments, at least one R^{6A} is a C₁₋₆ aliphatic group. In some embodiments, at least one R^{6A} is a C₁₋₆ aliphatic-cyclic group. In some embodiments, at least one R^{6A} is a 3-8 membered saturated or partially unsaturated monocyclic carbocyclic ring. In some embodiments, at least one R^{6A} is phenyl. In some embodiments, at least one R^{6A} is a 3-8 membered saturated or partially unsaturated monocyclic heterocyclic ring having 1-2 heteroatoms independently selected from nitrogen, oxygen, and sulfur. In some embodiments, at least one R^{6A} is a 5-6 membered monocyclic heteroaromatic ring having 1-4 heteroatoms independently selected from nitrogen, oxygen, and sulfur. In some embodiments, at least one R^{6A} is an 8-10 membered bicyclic heteroaromatic ring having 1-5 heteroatoms independently selected from nitrogen, oxygen, and sulfur. In some embodiments, at least one R^{6A} is a 7-12 membered saturated or partially unsaturated bicyclic heterocyclic ring having 1-4 heteroatoms independently selected from nitrogen, oxygen, and sulfur. In some embodiments, at least one R^{6A} is a 5-12 membered saturated or partially unsaturated bicyclic carbocyclic ring that is optionally bridged or spirocyclic. In some embodiments, at least one R^{6A} is a 5-12 membered saturated or partially unsaturated bicyclic heterocyclic ring that is optionally bridged or spirocyclic having 1-4 heteroatoms independently selected from nitrogen, oxygen, and sulfur. In some embodiments, there is only one R^{6A} (i.e., c is 1). In specific cases of that embodiment, the one R^{6A} is a C₁₋₆ aliphatic group. In specific cases of that embodiment, the one R^{6A} is a C₁₋₆ aliphatic-cyclic group. In specific cases of that embodiment, the one R^{6A} is a 3-8 membered saturated or partially unsaturated monocyclic carbocyclic ring. In specific cases of that embodiment, the one R^{6A} is phenyl. In specific cases of that embodiment, the one R^{6A} is a 3-8 membered saturated or partially unsaturated monocyclic heterocyclic ring having 1-2 heteroatoms independently selected from nitrogen, oxygen, and sulfur. In specific cases of that embodiment, the one R^{6A} is a 5-6 membered monocyclic heteroaromatic ring having 1-4 heteroatoms independently selected from nitrogen, oxygen, and sulfur. In specific cases of that embodiment, the one R^{6A} is an 8-10 membered bicyclic heteroaromatic ring having 1-5 heteroatoms independently selected from nitrogen, oxygen, and sulfur. In specific cases of that embodiment, the one R^{6A} is a 7-12 membered saturated or partially unsaturated bicyclic heterocyclic ring having 1-4 heteroatoms independently selected from nitrogen, oxygen, and sulfur. In specific cases of that embodiment, the one R^{6A} is a 5-12 membered saturated or partially unsaturated bicyclic carbocyclic ring that is optionally bridged or spirocyclic. In specific cases of that embodiment, the one R^{6A} is a 5-12 membered saturated or partially unsaturated bicyclic heterocyclic ring that is optionally bridged or spirocyclic having 1-4 heteroatoms independently selected from nitrogen, oxygen, and sulfur.

In some embodiments, at least one (or when c is 1, the only) R^{6A} is a 5-6 membered monocyclic heteroaromatic ring having 1-4 heteroatoms independently selected from nitrogen, oxygen, and sulfur, or a 9-membered bicyclic heteroaromatic ring having 1-5 heteroatoms independently selected from nitrogen, oxygen, and sulfur.

In some embodiments, at least one (or when c is 1, the only) R^{6A} is a 5-6 membered monocyclic heteroaromatic ring having 1-3 nitrogen and 0-1 sulfur heteroatoms or a 9-membered bicyclic heteroaromatic ring having 1-2 nitrogen heteroatoms.

In some embodiments, at least one (or when c is 1, the only) R^{6A} is substituted with y instances of R^{A}. In some cases, y is 0, i.e., R^{6A} is unsubstituted. In some cases, y is 1 or 2.

In some embodiments, at least one (or when c is 1, the only) R^{6A} is

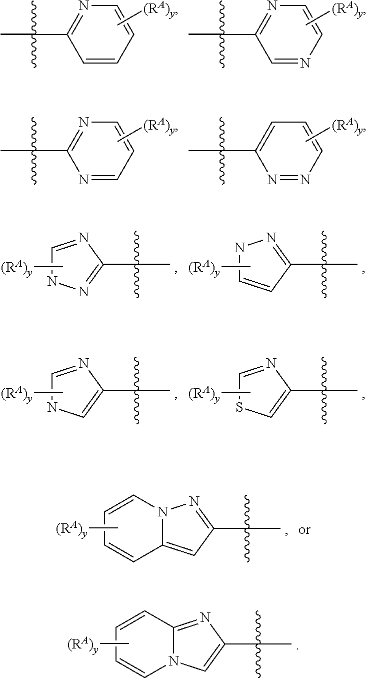

In some embodiments, at least one (or when c is 1, the only) R^{6A} is

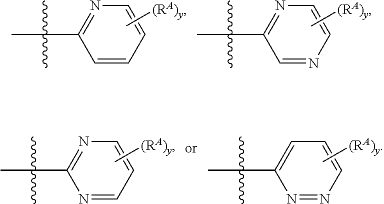

In some embodiments, at least one (or when c is 1, the only) R^{6A} is as depicted in the compounds of Table 1, below.

In some embodiments, at least one (or when c is 1, the only) R^{6A} and its R^{A} substituents is

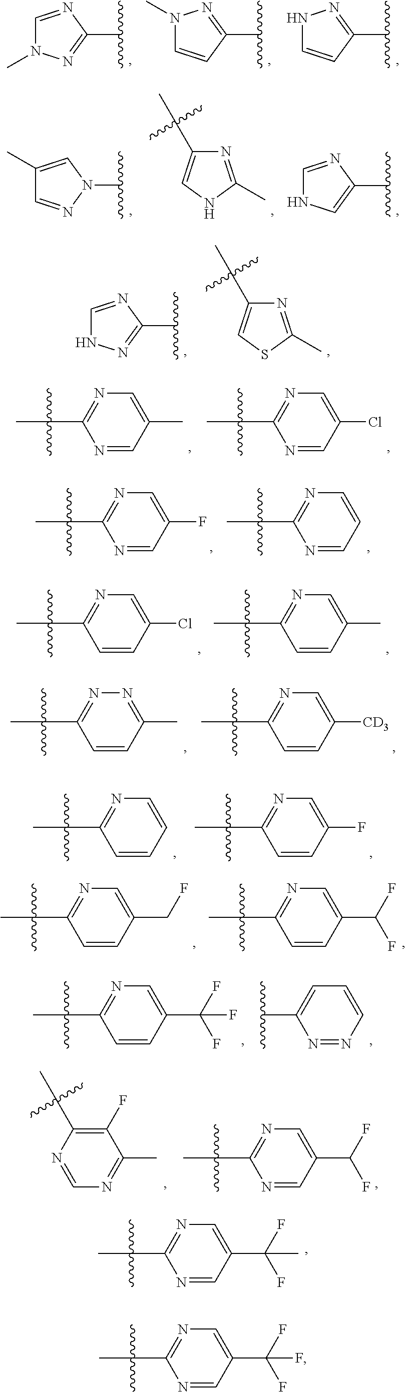

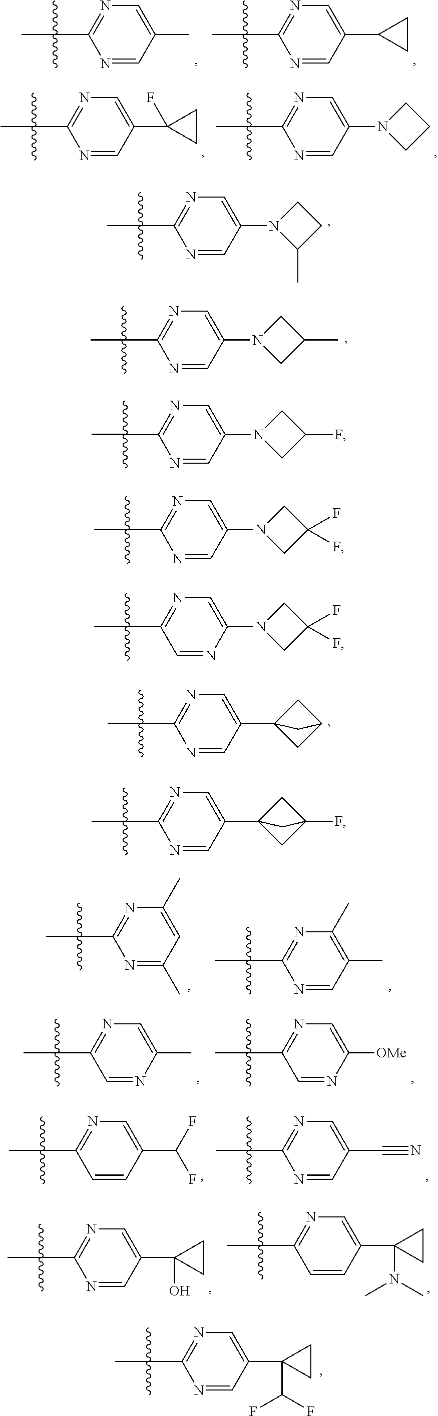

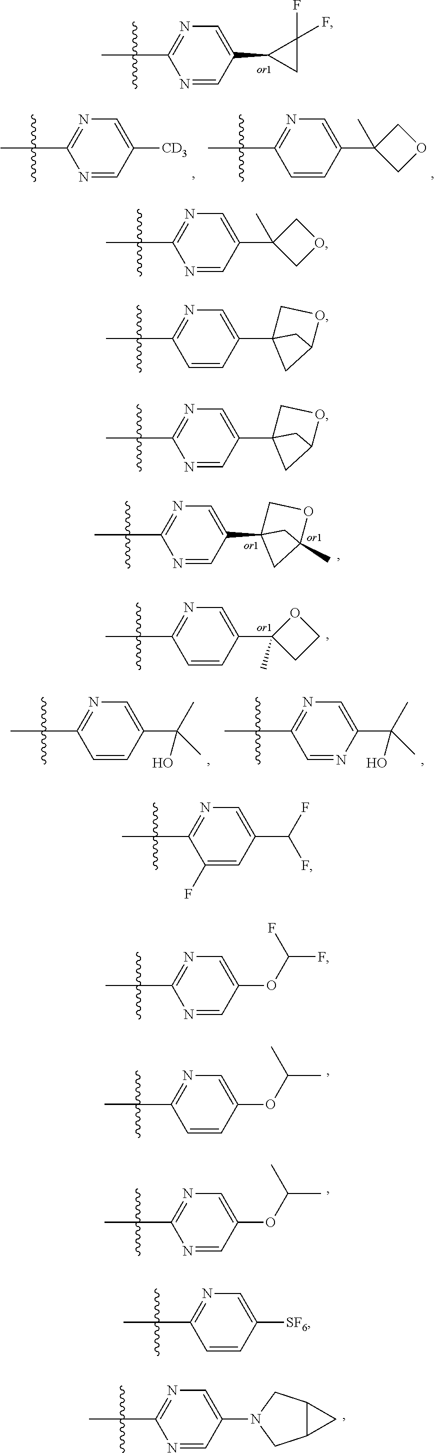

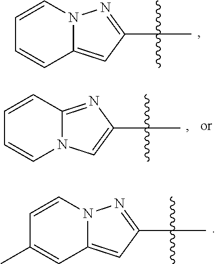

In some embodiments, at least one (or when c is 1, the only) R^{6A} and its R^{A} substituents is as depicted in the compounds of Table 1, below.

As defined generally above, c is 0, 1, 2 or 3.

In some embodiments, c is 0. In some embodiments, c is 1. In some embodiments, c is 2. In some embodiments, c is 3.

In some embodiments, c is 1 or 2. In some embodiments, c is 2 or 3.

In some embodiments, c is depicted in the compounds of Table 1, below.

As defined generally above, each R^{6B} is independently halogen, oxo, —OR, —SR, —NR₂, —SO₂R, —SO₂NR₂, —S(O)R, —S(O)NR₂, —C(O)R, —CO₂R, —C(O)NR₂, —C(O)N(R)OR, —OC(O)R, —OC(O)NR₂, —N(R)CO₂R, —N(R)C(O)R, —N(R)C(O)NR₂, —N(R)C(NR)R, —N(R)C(NR)NR₂, —N(R)NR₂, —N(R)SO₂NR₂, —N(R)SO₂R, —N═S(O)R₂, —S(NR)(O)R, —N(R)S(O)R, —N(R)CN, —P(O)(OR)₂, —P(O)R₂, or an optionally substituted group selected from C₁₋₆ aliphatic; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; and a 5-6 membered monocyclic heteroaryl ring having 1-4 heteroatoms independently selected from nitrogen, oxygen, and sulfur.

In some embodiments, at least one R^{6B} is halogen. In some embodiments, at least one R^{6B} is oxo. In some embodiments, at least one R^{6B} is —OR. In some embodiments, at least one R^{6B} is —SR. In some embodiments, at least one R^{6B} is —NR₂. In some embodiments, at least one R^{6B} is —SO₂R. In some embodiments, at least one R^{6B} is —SO₂NR₂. In some embodiments, at least one R^{6B} is —S(O)R. In some embodiments, at least one R^{6B} is —S(O)NR₂. In some embodiments, at least one R^{6B} is —C(O)R. In some embodiments, at least one R^{6B} is —CO₂R. In some embodiments, at least one R^{B} is —C(O)NR₂. In some embodiments, at least one R^{6B} is —C(O)N(R)OR. In some embodiments, at least one R^{6B} is —OC(O)R. In some embodiments, at least one R^{B} is —OC(O)NR₂. In some embodiments, at least one R^{6B} is —N(R)CO₂R. In some embodiments, at least one R^{6B} is —N(R)C(O)R. In some embodiments, at least one R^{6B} is —N(R)C(O)NR₂. In some embodiments, at least one R^{6B} is —N(R)C(NR)R. In some embodiments, at least one R^{6B} is —N(R)C(NR)NR₂. In some embodiments, at least one R^{6B} is —N(R)NR₂. In some embodiments, at least one R^{6B} is —N(R)SO₂NR₂. In some embodiments, at least one R^{B} is —N(R)SO₂R. In some embodiments, at least one R^{6B} is —N═S(O)R₂. In some embodiments, at least one R^{6B} is —S(NR)(O)R. In some embodiments, at least one R^{B} is —N(R)S(O)R. In some embodiments, at least one R^{6B} is —N(R)CN. In some embodiments, at least one R^{B} is —P(O)(OR)₂. In some embodiments, at least one R^{6B} is —P(O)R₂. In some embodiments, at least one R^{6B} is an optionally substituted group selected from C₁₋₆ aliphatic. In some embodiments, at least one R^{6B} is a 3-7 membered saturated or partially unsaturated monocyclic carbocyclic ring. In some embodiments, at least one R^{6B} is a 3-7 membered saturated or partially unsaturated monocyclic heterocyclic ring having 1-2 heteroatoms independently selected from nitrogen, oxygen, and sulfur. In some embodiments, at least one R^{B} is a 5-6 membered monocyclic heteroaryl ring having 1-4 heteroatoms independently selected from nitrogen, oxygen, and sulfur. In some embodiments, there is only one R^{6B} (i.e., b is 1). In specific cases of that embodiment, the one R^{6B} is halogen. In specific cases of that embodiment, the one R^{6B} is oxo. In specific cases of that embodiment, the one R^{6B} is —OR. In specific cases of that embodiment, the one R^{6B} is —SR. In specific cases of that embodiment, the one R^{6B} is —NR₂. In specific cases of that embodiment, the one R^{B} is —SO₂R. In specific cases of that embodiment, the one R^{6B} is —SO₂NR₂. In specific cases of that embodiment, the one R^{6B} is —S(O)R. In specific cases of that embodiment, the one R^{6B} is —S(O)NR₂. In specific cases of that embodiment, the one R^{6B} is —C(O)R. In specific cases of that embodiment, the one R^{6B} is —CO₂R. In specific cases of that embodiment, the one R^{B} is —C(O)NR₂. In specific cases of that embodiment, the one R^{6B} is —C(O)N(R)OR. In specific cases of that embodiment, the one R^{6B} is —OC(O)R. In specific cases of that embodiment, the one R^{B} is —OC(O)NR₂. In specific cases of that embodiment, the one R^{6B} is —N(R)CO₂R. In specific cases of that embodiment, the one R^{6B} is —N(R)C(O)R. In specific cases of that embodiment, the one R^{6B} is —N(R)C(O)NR₂. In specific cases of that embodiment, the one R^{6B} is —N(R)C(NR)R. In specific cases of that embodiment, the one R^{6B} is —N(R)C(NR)NR₂. In specific cases of that embodiment, the one R^{B} is —N(R)NR₂. In specific cases of that embodiment, the one R^{6B} is —N(R)SO₂NR₂. In specific cases of that embodiment, the one R^{6B} is —N(R)SO₂R. In specific cases of that embodiment, the one R^{6B} is —N═S(O)R₂. In specific cases of that embodiment, the one R^{6B} is —S(NR)(O)R. In specific cases of that embodiment, the one R^{6B} is —N(R)S(O)R. In specific cases of that embodiment, the one R^{6B} is —N(R)CN. In specific cases of that embodiment, the one R^{B} is —P(O)(OR)₂. In specific cases of that embodiment, the one R^{6B} is —P(O)R₂. In specific cases of that embodiment, the one R^{6B} is an optionally substituted group selected from C₁₋₆ aliphatic. In specific cases of that embodiment, the one R^{6B} is a 3-7 membered saturated or partially unsaturated monocyclic carbocyclic ring. In specific cases of that embodiment, the one R^{6B} is a 3-7 membered saturated or partially unsaturated monocyclic heterocyclic ring having 1-2 heteroatoms independently selected from nitrogen, oxygen, and sulfur. In specific cases of that embodiment, the one R^{6B} is a 5-6 membered monocyclic heteroaryl ring having 1-4 heteroatoms independently selected from nitrogen, oxygen, and sulfur.

In some embodiments, at least one (or when b is 1, the only) R^{6B} is hydrogen. In some embodiments, at least one (or when b is 1, the only) R^{6B} is C₁₋₄ alkyl. In some embodiments, at least one (or when b is 1, the only) R^{6B} is C₁₋₄ haloalkyl. In some embodiments, at least one (or when b is 1, the only) R^{6B} is oxo.

In some embodiments, at least one (or when b is 1, the only) R^{6B} is hydrogen, F, Cl, CH₃, or oxo.

In some embodiments, at least one (or when b is 1, the only) R^{6B} is F.

In some embodiments, at least one (or when b is 1, the only) R^{6B} is Cl.

In some embodiments, at least one (or when b is 1, the only) R^{6B} is CH₃.

In some embodiments, at least one (or when b is 1, the only) R^{6B} is as depicted in the compounds of Table 1, below.

As defined generally above, b is 0, 1, 2, or 3.

In some embodiments, b is 0. In some embodiments, b is 1. In some embodiments, b is 2. In some embodiments, b is 3.

In some embodiments, b is 1 or 2. In some embodiments, b is 2 or 3.

In some embodiments, b is depicted in the compounds of Table 1, below.

In some embodiments, each R^{A} is independently selected from deuterium, a C₁₋₆ aliphatic group substituted with 0, 1, 2, or 3 groups independently selected from halogen, deuterium, —CN, —NR₂, and —OR; a 5-6 membered monocyclic heteroaromatic ring having 1-4 heteroatoms independently selected from nitrogen, oxygen, and sulfur substituted with 0, 1, 2, or 3 groups independently selected from halogen, C₁₋₃ alkyl, C₁₋₃ haloalkyl, —CN, —NR₂, and —OR; a 3-8 membered saturated or partially unsaturated monocyclic heterocyclic ring having 1-2 heteroatoms independently selected from nitrogen, oxygen, and sulfur substituted with 0, 1, 2, or 3 groups independently selected from halogen, C₁₋₃ alkyl, C₁₋₃ haloalkyl, —CN, —NR₂, and —OR; a 3-8 membered saturated or partially unsaturated monocyclic carbocyclic ring substituted with 0, 1, 2, or 3 groups independently selected from halogen, C₁₋₃ alkyl, C₁₋₃ haloalkyl, —CN, —NR₂, and —OR; a 4-12 membered saturated or partially unsaturated bicyclic ring system that is fused, bridged, or spirocyclic selected from carbocyclyl and heterocyclyl (having 1-4 heteroatoms independently selected from nitrogen, oxygen, and sulfur), wherein said carbocyclyl or heterocyclyl is substituted with 0, 1, 2, or 3 groups independently selected C₁₋₃ alkyl, C₁₋₃ alkoxy, C₁₋₃ haloalkyl, C₁₋₃ hydroxyalkyl, halogen, —CN, —OR, and —NR₂; halogen; —CN; —NO₂; —OR; —SR; —NR₂; —S(O)₂R; —S(O)₂NR₂; —S(O)R; —S(O)NR₂; —C(O)R; —C(O)OR; —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —S(NR)(O)R; —N(R)S(O)₂NR₂; and —N(R)S(O)₂R; or two R^{A} on the same atom together form oxo or a cyclic group selected from:
a 3-8 membered saturated or partially unsaturated monocyclic carbocyclic ring; and
a 3-8 membered saturated or partially unsaturated monocyclic heterocyclic ring having 1-2 heteroatoms independently selected from nitrogen, oxygen, and sulfur;
wherein the cyclic group formed by two R^{A} on the same atom is substituted with 0, 1, 2, or 3 groups independently selected from a C₁₋₆ aliphatic group substituted with 0, 1, 2, or 3 groups independently selected from halogen, —CN, —NR₂, and —OR; halogen; —CN; —NO₂; —OR; —SR; —NR₂; —S(O)₂R; —S(O)₂NR₂; —S(O)R; —S(O)NR₂; —C(O)R; —C(O)OR; —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —N(R)S(O)₂NR₂; and —N(R)S(O)₂R; or
two R^{A} on adjacent atoms form a cyclic group selected from:
a 3-8 membered saturated or partially unsaturated monocyclic carbocyclic ring;
a 3-8 membered saturated or partially unsaturated monocyclic heterocyclic ring having 1-2 heteroatoms independently selected from nitrogen, oxygen, and sulfur;
phenyl; and
a 5-6 membered heteroaryl ring having 1-4 heteroatoms independently selected from nitrogen, oxygen, and sulfur,
   wherein the cyclic group formed by two R^{A} on adjacent atoms is substituted with 0, 1, 2, or 3 groups independently selected from a C₁₋₆ aliphatic group substituted with 0, 1, 2, or 3 groups independently selected from halogen, —CN, —NR₂, and —OR; halogen; —CN; —NO₂; —OR; —SR; —NR₂; —S(O)₂R; —S(O)₂NR₂; —S(O)R; —S(O)NR₂; —C(O)R; —C(O)OR; —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —N(R)S(O)₂NR₂; and —N(R)S(O)₂R.

In some embodiments, at least one R^{A} is deuterium. In some embodiments, at least one R^{A} is a C₁₋₆ aliphatic group substituted with 0, 1, 2, or 3 groups independently selected from halogen, deuterium, —CN, —NR₂, and —OR. In some embodiments, at least one R^{A} is a 5-6 membered monocyclic heteroaromatic ring having 1-4 heteroatoms independently selected from nitrogen, oxygen, and sulfur substituted with 0, 1, 2, or 3 groups independently selected from halogen, C₁₋₃ alkyl, C₁₋₃ haloalkyl, —CN, —NR₂, and —OR. In some embodiments, at least one R^{A} is a 3-8 membered saturated or partially unsaturated monocyclic heterocyclic ring having 1-2 heteroatoms independently selected from nitrogen, oxygen, and sulfur substituted with 0, 1, 2, or 3 groups independently selected from halogen, C₁₋₃ alkyl, C₁₋₃ haloalkyl, —CN, —NR₂, and —OR. In some embodiments, at least one R^{A} is a 3-8 membered saturated or partially unsaturated monocyclic carbocyclic ring substituted with 0, 1, 2, or 3 groups independently selected from halogen, C₁₋₃ alkyl, C₁₋₃ haloalkyl, —CN, —NR₂, and —OR. In some embodiments, at least one R^{A} is a 4-12 membered saturated or partially unsaturated bicyclic ring system that is fused, bridged, or spirocyclic selected from carbocyclyl and heterocyclyl (having 1-4 heteroatoms independently selected from nitrogen, oxygen, and sulfur), wherein said carbocyclyl or heterocyclyl is substituted with 0, 1, 2, or 3 groups independently selected C₁₋₃ alkyl, C₁₋₃ alkoxy, C₁₋₃ haloalkyl, C₁₋₃ hydroxyalkyl, halogen, —CN, —OR, and —NR₂. In some embodiments, at least one R^{A} is halogen. In some embodiments, at least one R^{A} is —CN. In some embodiments, at least one R^{A} is —NO₂. In some embodiments, at least one R^{A} is —OR. In some embodiments, at least one R^{A} is —SR. In some embodiments, at least one R^{A} is —NR₂. In some embodiments, at least one R^{A} is —S(O)₂R. In some embodiments, at least one R^{A} is —S(O)₂NR₂. In some embodiments, at least one R^{A} is —S(O)R. In some embodiments, at least one R^{A} is —S(O)NR₂. In some embodiments, at least one R^{A} is —C(O)R. In some embodiments, at least one R^{A} is —C(O)OR. In some embodiments, at least one R^{A} is —C(O)NR₂. In some embodiments, at least one R^{A} is —C(O)N(R)OR. In some embodiments, at least one R^{A} is —OC(O)R. In some embodiments, at least one R^{A} is —OC(O)NR₂. In some embodiments, at least one R^{A} is —N(R)C(O)OR. In some embodiments, at least one R^{A} is —N(R)C(O)R. In some embodiments, at least one R^{A} is —N(R)C(O)NR₂. In some embodiments, at least one R^{A} is —N(R)C(NR)NR₂. In some embodiments, at least one R^{A} is —S(NR)(O)R. In some embodiments, at least one R^{A} is —N(R)S(O)₂NR₂. In some embodiments, at least one R^{A} is —N(R)S(O)₂R. In some embodiments, two R^{A} on the same atom together form oxo. In some embodiments, two R^{A} on the same atom together form a cyclic group. In some embodiments, two R^{A} on the same atom together form a 3-8 membered saturated or partially unsaturated monocyclic carbocyclic ring. In some embodiments, two R^{A} on the same atom together form a 3-8 membered saturated or partially unsaturated monocyclic heterocyclic ring having 1-2 heteroatoms independently selected from nitrogen, oxygen, and sulfur. In some embodiments, the cyclic group formed by two R^{A} on the same atom is substituted with 0, 1, 2, or 3 groups independently selected from a C₁₋₆ aliphatic group substituted with 0, 1, 2, or 3 groups independently selected from halogen, —CN, —NR₂, and —OR; halogen; —CN; —NO₂; —OR; —SR; —NR₂; —S(O)₂R; —S(O)₂NR₂; —S(O)R; —S(O)NR₂; —C(O)R; —C(O)OR; —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —N(R)S(O)₂NR₂; and —N(R)S(O)₂R. In some embodiments, two R^{A} on adjacent atoms form a cyclic group. In some embodiments, two R^{A} on adjacent atoms form a 3-8 membered saturated or partially unsaturated monocyclic carbocyclic ring. In some embodiments, two R^{A} on adjacent atoms form a 3-8 membered saturated or partially unsaturated monocyclic heterocyclic ring having 1-2 heteroatoms independently selected from nitrogen, oxygen, and sulfur. In some embodiments, two R^{A} on adjacent atoms form a phenyl. In some embodiments, two R^{A} on adjacent atoms form a 5-6 membered heteroaryl ring having 1-4 heteroatoms independently selected from nitrogen, oxygen, and sulfur. In some embodiments, the cyclic group formed by two R^{A} on adjacent atoms is substituted with 0, 1, 2, or 3 groups independently selected from a C₁₋₆ aliphatic group substituted with 0, 1, 2, or 3 groups independently selected from halogen, —CN, —NR₂, and —OR; halogen; —CN; —NO₂; —OR; —SR; —NR₂; —S(O)₂R; —S(O)₂NR₂; —S(O)R; —S(O)NR₂; —C(O)R; —C(O)OR; —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —N(R)S(O)₂NR₂; and —N(R)S(O)₂R.

In some embodiments, at least one (or, when y is 1, the only) R^{A} is halogen; —CN; —OR; a C₁₋₆ aliphatic group substituted with 0, 1, 2, or 3 groups independently selected from halogen, deuterium, —CN, —NR₂, and —OR; a 3-8 membered saturated or partially unsaturated monocyclic heterocyclic ring having 1-2 heteroatoms independently selected from nitrogen, oxygen, and sulfur substituted with 0, 1, 2, or 3 groups independently selected from halogen, C₁₋₃ alkyl, C₁₋₃ haloalkyl, —CN, —NR₂, and —OR; or a 4-12 membered saturated or partially unsaturated bicyclic ring system that is fused, bridged, or spirocyclic selected from carbocyclyl and heterocyclyl (having 1-4 heteroatoms independently selected from nitrogen, oxygen, and sulfur), wherein said carbocyclyl or heterocyclyl is substituted with 0, 1, 2, or 3 groups independently selected C₁₋₃ alkyl, C₁₋₃ alkoxy, C₁₋₃ haloalkyl, C₁₋₃ hydroxyalkyl, halogen, —CN, —OR, and —NR₂.

In some embodiments, at least one (or, when y is 1, the only) R^{A} is fluoro, chloro, —CN, methyl, —CD₃, —CH₂F, —CHF₂, —CF₃, —C(CH₃)OH, cyclopropyl, —OMe, —OiPr, —OCHF₂, —SF₆,

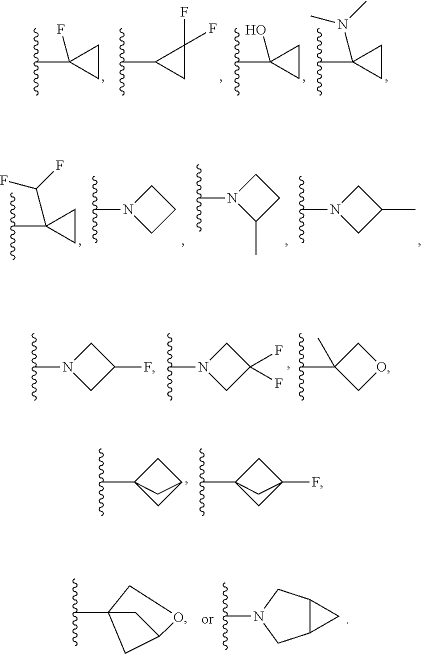

In some embodiments, at least one (or, when y is 1, the only) R^{A} is as depicted in the compounds of Table 1, below.

As defined generally above, each R¹ is independently selected from the group consisting of hydrogen, C₁₋₃ haloalkyl, C₁₋₃ hydroxyalkyl, C₀₋₃ alkyl-C₃-C₆ cycloalkyl, C₀₋₃ alkyl-C₃-C₆ heterocycloalkyl (having 1-4 heteroatoms independently selected from nitrogen, oxygen, and sulfur), and C₁₋₃ alkyl; wherein C₀₋₃ alkyl-C₃-C₆ cycloalkyl, C₀₋₃ alkyl-C₃-C₆ heterocycloalkyl and C₁₋₃ alkyl are substituted with y instances of R^{B}.

In some embodiments, at least one or each R¹ is hydrogen. In some embodiments, at least one R¹ is C₁₋₃ haloalkyl. In some embodiments, at least one or each R¹ is C₁₋₃ hydroxyalkyl. In some embodiments, at least one or each R¹ is C₀₋₃ alkyl-C₃-C₆ cycloalkyl substituted with y instances of R^{B}. In some embodiments, at least one or each R¹ is C₀₋₃ alkyl-C₃-C₆ heterocycloalkyl (having 1-4 heteroatoms independently selected from nitrogen, oxygen, and sulfur) substituted with y instances of R^{B}. In some embodiments, at least one or each R¹ is C₁₋₃ alkyl substituted with y instances of R^{B}.

In some embodiments, at least one or each R¹ is depicted in the compounds of Table 1, below.

As defined generally above, each R^{B} is independently halogen, C₁₋₃ alkyl, hydroxy-C₁₋₃ alkyl, —CN, oxo, —OR, or —NR₂.

In some embodiments, at least one or each R^{B} is halogen. In some embodiments, at least one or each R^{B} is C₁₋₃ alkyl. In some embodiments, at least one or each R^{B} is hydroxy-C₁₋₃ alkyl. In some embodiments, at least one or each R^{B} is —CN. In some embodiments, at least one or each R^{B} is oxo. In some embodiments, at least one or each R^{B} is —OR. In some embodiments, at least one or each R^{B} is —NR₂.

In some embodiments, at least one or each R^{B} is depicted in the compounds of Table 1, below.

As defined generally above, each y is independently 0, 1, 2, or 3.

In some embodiments, y is 0. In some embodiments, y is 1. In some embodiments, y is 2. In some embodiments, y is 3. In some embodiments, y is 0 or 1. In some embodiments, y is 1 or 2. In some embodiments, y is 2 or 3.

In some embodiments, each y is depicted in the compounds of Table 1, below.

As defined generally above, each R^{1A} are independently halogen, —CN, oxo, —OR, —SR, —NR₂, —SO₂R, —SO₂NR₂, —S(O)R, —S(O)NR₂, —C(O)R, —CO₂R, —C(O)NR₂, —C(O)N(R)OR, —OC(O)R, —OC(O)NR₂, —N(R)CO₂R, —N(R)C(O)R, —N(R)C(O)NR₂, —N(R)C(NR)R, —N(R)C(NR)NR₂, —N(R)NR₂, —N(R)SO₂NR₂, —N(R)SO₂R, —N═S(O)R₂, —S(NR)(O)R, —N(R)S(O)R, —N(R)CN, —P(O)(OR)₂, —P(O)R₂, C₁-C₆ haloalkyl, C₁₋₃ hydroxyalkyl, C₃-C₆ halocycloalkyl, C₁-C₆ haloalkoxy, or an optionally substituted group selected from C₁₋₆ aliphatic; phenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; and a 5-6 membered monocyclic heteroaryl ring having 1-4 heteroatoms independently selected from nitrogen, oxygen, and sulfur.

In some embodiments, at least one (or when a is 1, the only) R^{1A} is CH₂CH₃. In some embodiments, at least one (or when a is 1, the only) R^{1A} is CH₃. In some embodiments, at least one (or when a is 1, the only) R^{1A} is CF₃. In some embodiments, at least one (or when a is 1, the only) R^{1A} is CHF₂. In some embodiments, at least one (or when a is 1, the only) R^{1A} is CH₂CF₃. In some embodiments, at least one (or when a is 1, the only) R^{1A} is CH₂CHF₂.

In some embodiments, each R^{1A} is as depicted in the compounds of Table 1, below.

In some embodiments, Ring C together with its R^{1A} and L₅ substituents is as depicted in the compounds of Table 1, below.

In some embodiments, at least one (or when a is 1, the only) R^{1A} is halogen. In some embodiments, at least one (or when a is 1, the only) R^{1A} is —CN. In some embodiments, at least one (or when a is 1, the only) R^{1A} is oxo. In some embodiments, at least one (or when a is 1, the only) R^{1A} is —OR. In some embodiments, at least one (or when a is 1, the only) R^{1A} is —SR. In some embodiments, at least one (or when a is 1, the only) R^{1A} is —NR₂. In some embodiments, at least one (or when a is 1, the only) R^{1A} is —SO₂R. In some embodiments, at least one (or when a is 1, the only) R^{1A} is —SO₂NR₂. In some embodiments, at least one (or when a is 1, the only) R^{1A} is —S(O)R. In some embodiments, at least one (or when a is 1, the only) R^{1A} is —S(O)NR₂. In some embodiments, at least one (or when a is 1, the only) R^{1A} is —C(O)R. In some embodiments, at least one (or when a is 1, the only) R^{1A} is —CO₂R. In some embodiments, at least one (or when a is 1, the only) R^{1A} is —C(O)NR₂. In some embodiments, at least one (or when a is 1, the only) R^{1A} is —C(O)N(R)OR. In some embodiments, at least one (or when a is 1, the only) R^{1A} is —OC(O)R. In some embodiments, at least one (or when a is 1, the only) R^{1A} is —OC(O)NR₂. In some embodiments, at least one (or when a is 1, the only) R^{1A} is —N(R)CO₂R. In some embodiments, at least one (or when a is 1, the only) R^{1A} is —N(R)C(O)R. In some embodiments, at least one (or when a is 1, the only) R^{1A} is —N(R)C(O)NR₂. In some embodiments, at least one (or when a is 1, the only) R^{1A} is —N(R)C(NR)R. In some embodiments, at least one (or when a is 1, the only) R^{1A} is —N(R)C(NR)NR₂. In some embodiments, at least one (or when a is 1, the only) R^{1A} is —N(R)NR₂. In some embodiments, at least one (or when a is 1, the only) R^{1A} is —N(R)SO₂NR₂. In some embodiments, at least one (or when a is 1, the only) R^{1A} is —N(R)SO₂R. In some embodiments, at least one (or when a is 1, the only) R^{1A} is —N═S(O)R₂. In some embodiments, at least one (or when a is 1, the only) R^{1A} is —S(NR)(O)R. In some embodiments, at least one (or when a is 1, the only) R^{1A} is —N(R)S(O)R. In some embodiments, at least one (or when a is 1, the only) R^{1A} is —N(R)CN. In some embodiments, at least one (or when a is 1, the only) R^{1A} is —P(O)(OR)₂. In some embodiments, at least one (or when a is 1, the only) R^{1A} is —P(O)R₂. In some embodiments, at least one (or when a is 1, the only) R^{1A} is C₁-C₆haloalkyl. In some embodiments, at least one (or when a is 1, the only) R^{1A} is C₁₋₃ hydroxyalkyl. In some embodiments, at least one (or when a is 1, the only) R^{1A} is C₃-C₆ halocycloalkyl. In some embodiments, at least one (or when a is 1, the only) R^{1A} is C₁-C₆ haloalkoxy. In some embodiments, at least one (or when a is 1, the only) R^{1A} is an optionally substituted C₁₋₆ aliphatic. In some embodiments, at least one (or when a is 1, the only) R^{1A} is an optionally substituted phenyl. In some embodiments, at least one (or when a is 1, the only) R^{1A} is an optionally substituted 3-7 membered saturated or partially unsaturated monocyclic carbocyclic ring. In some embodiments, at least one (or when a is 1, the only) R^{1A} is an optionally substituted 3-7 membered saturated or partially unsaturated monocyclic heterocyclic ring having 1-2 heteroatoms independently selected from nitrogen, oxygen, and sulfur. In some embodiments, at least one (or when a is 1, the only) R^{1A} is an optionally substituted 5-6 membered monocyclic heteroaryl ring having 1-4 heteroatoms independently selected from nitrogen, oxygen, and sulfur.

In some embodiments, at least one (or when a is 1, the only) R^{1A} is halogen, —CN, oxo, —OR, —SO₂R, —C(O)NR₂, an optionally substituted C₁₋₆ alkyl, a 3-7 membered saturated monocyclic carbocyclic ring, or an optionally substituted 3-7 membered saturated monocyclic heterocyclic ring having 1-2 heteroatoms independently selected from nitrogen, oxygen, and sulfur.

In some embodiments, at least one (or when a is 1, the only) R^{1A} is halogen, —CN, —C(O)NR₂, an optionally substituted C₁₋₆ alkyl, a 3-7 membered saturated monocyclic carbocyclic ring, or an optionally substituted 3-7 membered saturated monocyclic heterocyclic ring having 1-2 heteroatoms independently selected from nitrogen, oxygen, and sulfur.

In some embodiments, at least one (or when a is 1, the only) R^{1A} is fluoro, chloro, —CN, oxo, —OMe, —O(CH₂)₂NMe₂, —O(CH₂)₂NEt₂, —O(CH₂)₂pyrrolidinyl, —O(CH₂)₂piperidinyl, —SO₂cyclopropyl, —SO₂Ph, —C(O)NH(CH₂)₂NMe₂, —C(O)NH(CH₂)₃NMe₂, methyl, ethyl, propyl, isopropyl, isobutyl, tert-butyl, isopentyl, cyclopentyl, cyclobutyl, methyloxycyclobutyl, —CHF₂, —CF₃, —CH₂CH₂F, —CH₂CHF₂, —CH₂CF₃, —CH₂NHMe, —CH₂NMeCO₂tBu, —CH₂SO₂Me, —CH₂CO₂H, —CH₂CO₂Me, —CH₂CONHMe, —CH₂CONMe₂, —CH₂CN, —CH(Me)CN, —CH(Me)₂CN, —CH₂cyclobutyl, —CH₂(methyloxycyclobutyl), —CH₂Oxetanyl, —CH₂(methyloxetanyl), —CH₂(fluorooxetanyl), —CH₂azetidinyl, —CH₂tetrahydrofuranyl, —CH₂pyrrolidinyl, —CH₂(N-methylpyrrolidinyl), —CH₂pyrazolyl, —CH₂tetrahydro-2H-pyranyl, —CH₂morpholinyl, —CH₂(N-methylmorpholinyl), —CH₂(N-methylpiperazinyl), —CH₂(N,N-dimethylpiperazinyl), —CH₂(N-acetylpiperazinyl), —CH₂pyridyl, benzyl, —CH₂(cyclopropylenyl)CN, —(CH₂)₂pyrrolidin-2-onyl, —(CH₂)₂CN, —(CH₂)₂NH₂, —(CH₂)₂oxetanyl, —(CH₂)₂azetidinyl, —(CH₂)₃NMe₂, piperazinyl, or N-methylpiperazinyl.

In some embodiments, at least one (or when a is 1, the only) R^{1A} is fluoro, chloro, —CN, —C(O)NH(CH₂)₂NMe₂, —C(O)NH(CH₂)₃NMe₂, methyl, ethyl, propyl, isopropyl, isobutyl, tert-butyl, isopentyl, cyclopentyl, cyclobutyl, —CHF₂, —CF₃, piperazinyl, or N-methylpiperazinyl.

In some embodiments, each R^{1A} is as depicted in the compounds of Table 1, below.

As defined generally above, a is 0, 1, 2, or 3.

In some embodiments, a is 0. In some embodiments, a is 1. In some embodiments, a is 2. In some embodiments, a is 3.

In some embodiments, a is 1 or 2. In some embodiments, a is 2 or 3.

In some embodiments, a is depicted in the compounds of Table 1, below.

In some embodiments, Ring C together with its R^{1A} and L₅ substituent is

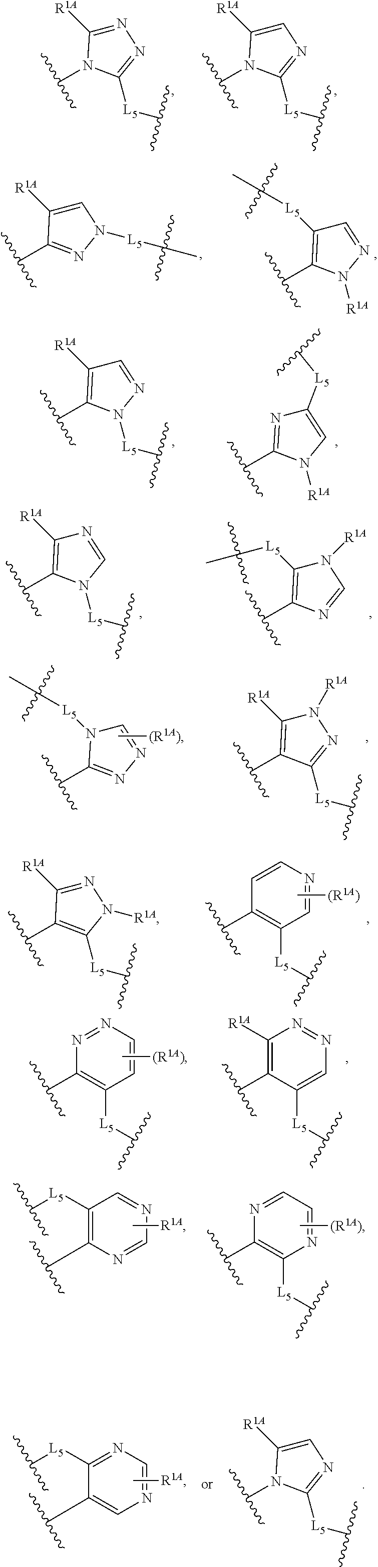

In some embodiments, Ring C together with its R^{1A} and L₅ substituents is

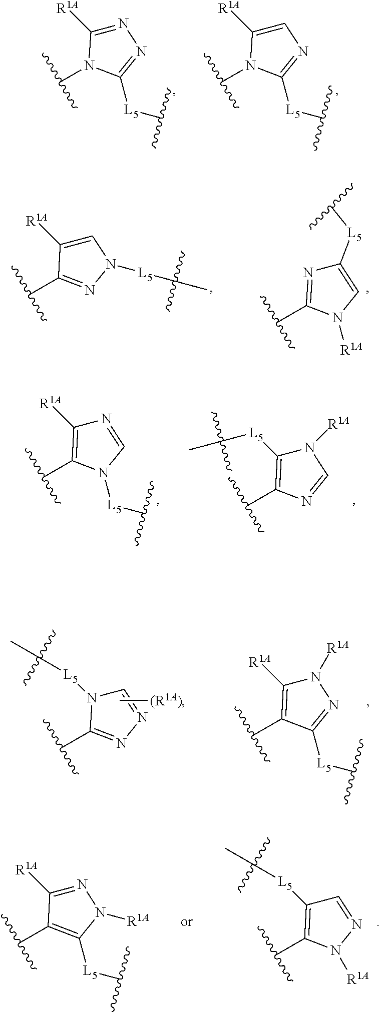

In some embodiments, Ring C together with its R^{1A} and L₅ substituent is as depicted in the compounds of Table 1, below.

As defined generally above, L₁ is selected from a bond, —O—, —S—, —S(O)₂—, —NR¹—, —C(O)—, —C(O)NR¹—, —NR¹C(O)—, —NR¹C(O)NR¹—, and —[C(R^{1a})(R^{1b})]₁₋₂—.

In some embodiments, L₁ is a bond. In some embodiments, L₁ is —O—. In some embodiments, L₁ is —S—. In some embodiments, L₁ is —S(O)₂—. In some embodiments, L₁ is —NR¹—. In some embodiments, L₁ is —C(O)—. In some embodiments, L₁ is —C(O)NR¹—. In some embodiments, L₁ is —NR¹C(O)—. In some embodiments, L₁ is —NR¹C(O)NR¹—. In some embodiments, L₁ is —[C(R^{1a})(R^{b})]₁₋₂—.

As defined generally above, L₂ is selected from a bond, a substituted or unsubstituted alkenylene, —O—, —S—, —S(O)₂—, —N═S(O)(CH₃)—, —S(O)(CH₃)═N—, —S(O)₂NR¹—, —NR¹S(O)₂—, —NR¹—, —C(O)—, —C(O)NR¹—, —NR¹C(O)—, —NR¹C(O)NR¹—, an optionally substituted C₃-C₆ cycloalkyl, an optionally substituted C₃-C₆ heterocycloalkyl (having 1-4 heteroatoms independently selected from nitrogen, oxygen, and sulfur), an optionally substituted 5-6 membered monocyclic heteroaryl ring having 1-3 heteroatoms independently selected from nitrogen, oxygen, and sulfur; and —[C(R^{2a})(R^{2b})]₁₋₂—.

In some embodiments, L₂ is a bond. In some embodiments, L₂ is a substituted or unsubstituted alkenylene. In some embodiments, L₂ is —O—. In some embodiments, L₂ is —S—. In some embodiments, L₂ is —S(O)₂—. In some embodiments, L₂ is —N═S(O)(CH₃)—. In some embodiments, L₂ is —S(O)(CH₃)═N—. In some embodiments, L₂ is —S(O)₂NR¹—. In some embodiments, L₂ is —NR¹S(O)₂—. In some embodiments, L₂ is —NR¹—. In some embodiments, L₂ is —C(O)—. In some embodiments, L₂ is —C(O)NR¹—. In some embodiments, L₂ is —NR¹C(O)—. In some embodiments, L₂ is —NR¹C(O)NR¹—. In some embodiments, L₂ is an optionally substituted C₃-C₆ cycloalkyl. In some embodiments, L₂ is an optionally substituted C₃-C₆ heterocycloalkyl (having 1-4 heteroatoms independently selected from nitrogen, oxygen, and sulfur). In some embodiments, L₂ is an optionally substituted 5-6 membered monocyclic heteroaryl ring having 1-3 heteroatoms independently selected from nitrogen, oxygen, and sulfur. In some embodiments, L₂ is —[C(R^{2a})(R^{2b})]₁₋₂—.

As defined generally above, L₃ is selected from a bond, a substituted or unsubstituted alkenylene, —O—, —S—, —S(O)₂—, —N═S(O)(CH₃)—, —S(O)(CH₃)═N—, —S(O)₂NR¹—, —NR¹S(O)₂—, —NR¹—, —C(O)—, —C(O)NR¹—, —NR¹C(O)—, —NR¹C(O)NR¹—, an optionally substituted C₃-C₆ cycloalkyl, an optionally substituted C₃-C₆ heterocycloalkyl (having 1-4 heteroatoms independently selected from nitrogen, oxygen, and sulfur), an optionally substituted 5-6 membered monocyclic heteroaryl ring having 1-3 heteroatoms independently selected from nitrogen, oxygen, and sulfur; and —[C(R^{3a})(R^{3b})]₁₋₂—.

In some embodiments, L₃ is a bond. In some embodiments, L₃ is a substituted or unsubstituted alkenylene. In some embodiments, L₃ is —O—. In some embodiments, L₃ is —S—. In some embodiments, L₃ is —S(O)₂—. In some embodiments, L₃ is —N═S(O)(CH₃)—. In some embodiments, L₃ is —S(O)(CH₃)═N—. In some embodiments, L₃ is —S(O)₂NR¹—. In some embodiments, L₃ is —NR¹S(O)₂—. In some embodiments, L₃ is —NR¹—. In some embodiments, L₃ is —C(O)—. In some embodiments, L₃ is —C(O)NR¹—. In some embodiments, L₃ is —NR¹C(O)—. In some embodiments, L₃ is —NR¹C(O)NR¹—. In some embodiments, L₃ is an optionally substituted C₃-C₆ cycloalkyl. In some embodiments, L₃ is an optionally substituted C₃-C₆ heterocycloalkyl (having 1-4 heteroatoms independently selected from nitrogen, oxygen, and sulfur). In some embodiments, L₃ is an optionally substituted 5-6 membered monocyclic heteroaryl ring having 1-3 heteroatoms independently selected from nitrogen, oxygen, and sulfur. In some embodiments, L₃ is —[C(R^{3a})(R^{3b})]₁₋₂—.

As defined generally above, L₄ is selected from a bond, a substituted or unsubstituted alkenylene, —O—, —S—, —S(O)₂—, —N═S(O)(CH₃)—, —S(O)(CH₃)═N—, —S(O)₂NR¹—, —NR¹S(O)₂—, —NR¹—, —C(O)—, —C(O)NR¹—, —NR¹C(O)—, —NR¹C(O)NR¹—, an optionally substituted C₃-C₆ cycloalkyl, an optionally substituted C₃-C₆ heterocycloalkyl (having 1-4 heteroatoms independently selected from nitrogen, oxygen, and sulfur), an optionally substituted 5-6 membered monocyclic heteroaryl ring having 1-3 heteroatoms independently selected from nitrogen, oxygen, and sulfur; and —[C(R^{4a})(R^{4b})]₁₋₂—.

In some embodiments, L₄ is a bond. In some embodiments, L₄ is a substituted or unsubstituted alkenylene. In some embodiments, L₄ is —O—. In some embodiments, L₄ is —S—. In some embodiments, L₄ is —S(O)₂—. In some embodiments, L₄ is —N═S(O)(CH₃)—. In some embodiments, L₄ is —S(O)(CH₃)═N—. In some embodiments, L₄ is —S(O)₂NR¹—. In some embodiments, L₄ is —NR¹S(O)₂—. In some embodiments, L₄ is —NR¹—. In some embodiments, L₄ is —C(O)—. In some embodiments, L₄ is —C(O)NR¹—. In some embodiments, L₄ is —NR¹C(O)—. In some embodiments, L₄ is —NR¹C(O)NR¹—. In some embodiments, L₄ is an optionally substituted C₃-C₆ cycloalkyl. In some embodiments, L₄ is an optionally substituted C₃-C₆ heterocycloalkyl (having 1-4 heteroatoms independently selected from nitrogen, oxygen, and sulfur). In some embodiments, L₄ is an optionally substituted 5-6 membered monocyclic heteroaryl ring having 1-3 heteroatoms independently selected from nitrogen, oxygen, and sulfur. In some embodiments, L₄ is —[C(R^{4a})(R^{4b})]₁₋₂—.

As defined generally above, L₅ is selected from a bond, —O—, —S—, —S(O)₂—, —NR¹—, —C(O)—, —C(O)NR¹—, —NR¹C(O)—, —NR¹C(O)NR¹—, and —[C(R^{5a})(R^{5b})]₁₋₂—.

In some embodiments, L₅ is a bond. In some embodiments, L₅ is —O—. In some embodiments, L₅ is —S—. In some embodiments, L₅ is —S(O)₂—. In some embodiments, L₅ is —NR¹—. In some embodiments, L₅ is —C(O)—. In some embodiments, L₅ is —C(O)NR¹—. In some embodiments, L₅ is —NR¹C(O)—. In some embodiments, L₅ is —NR¹C(O)NR¹—. In some embodiments, L₅ is —[C(R^{1a})(R^{1b})]₁₋₂—.

In some embodiments, L₁ is —CH₂— or —O— and L₅ is —CH₂— or —O—. In some embodiments, L₁ is —O— and L₅ is —CH₂—. In some embodiments, L₁ is —CH₂— and L₅ is —O—. In some embodiments, L₁ is —O— and L₅ is —O—.

In some embodiments, L₁, L₂, L₃, L₄, and L₅ together form

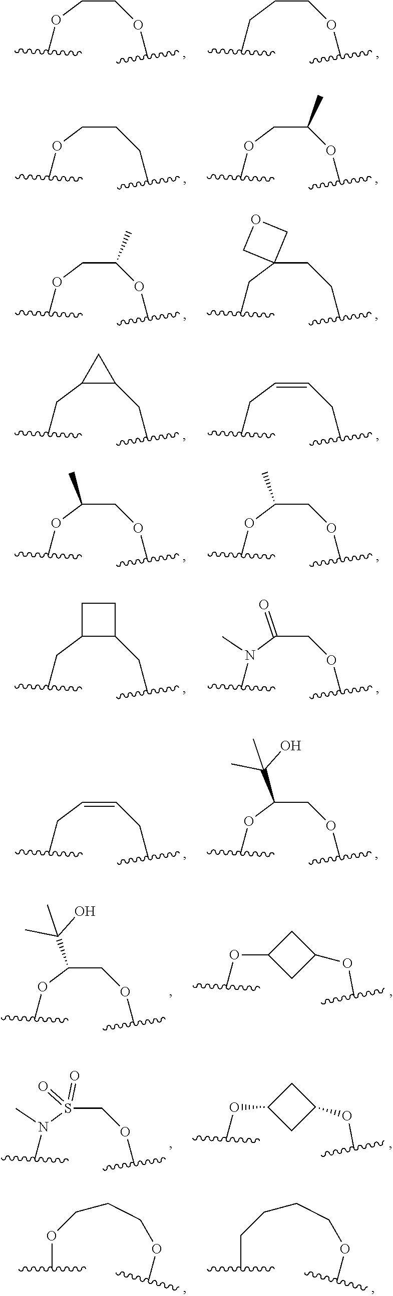

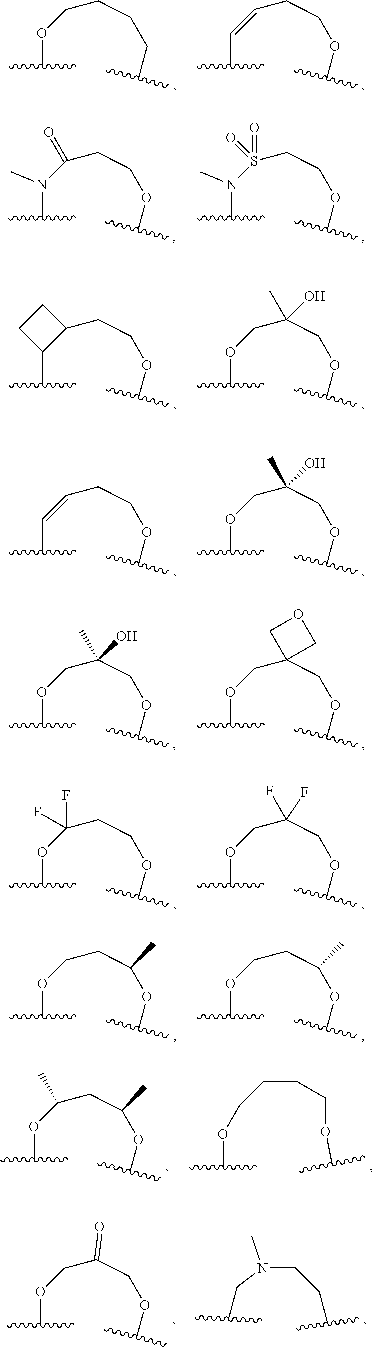

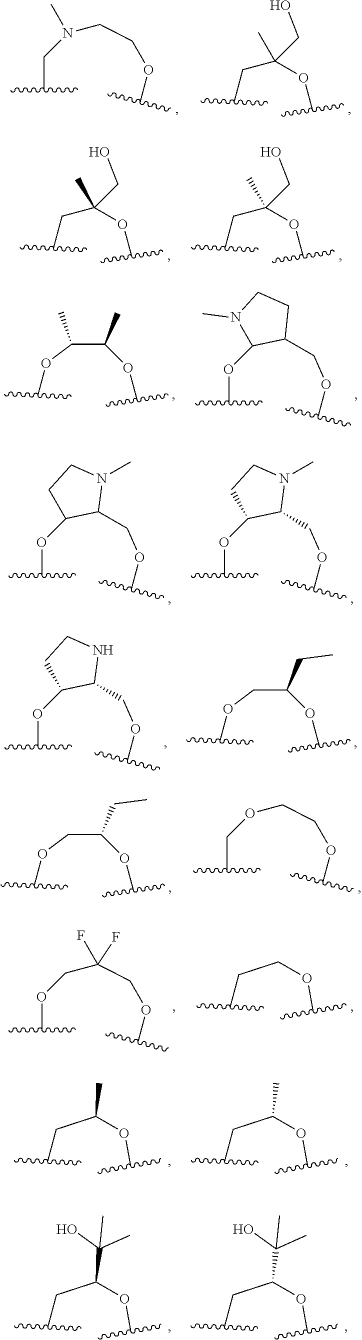

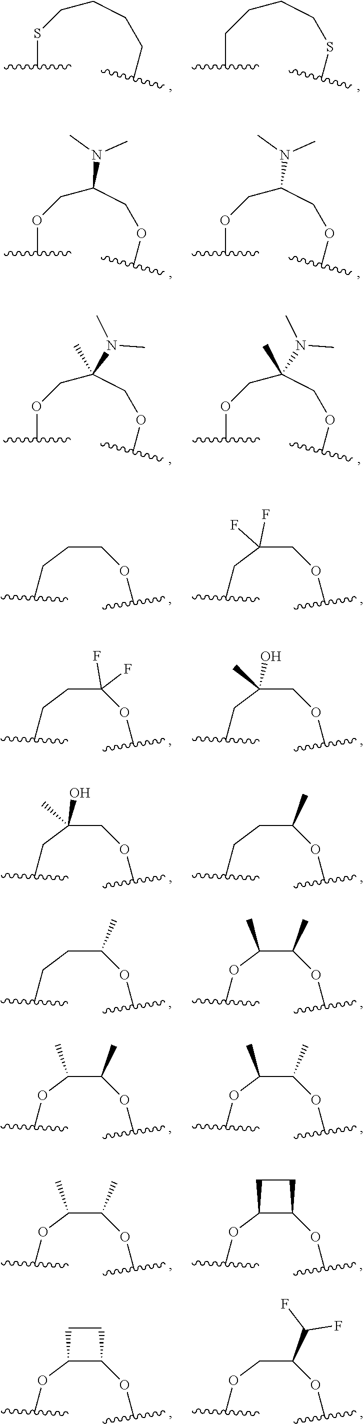

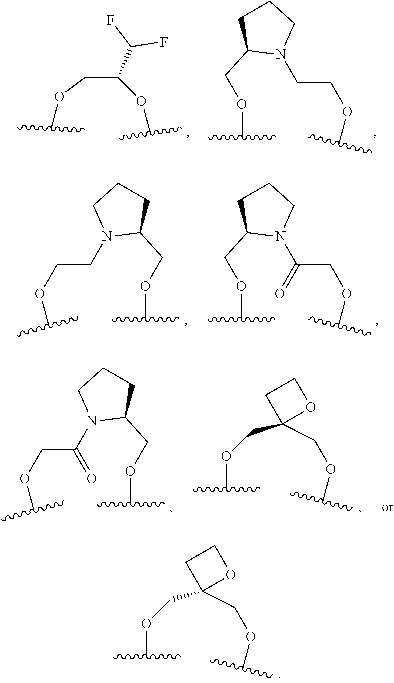

In some embodiments L₁, L₂, L₃, L₄ and L₅ form

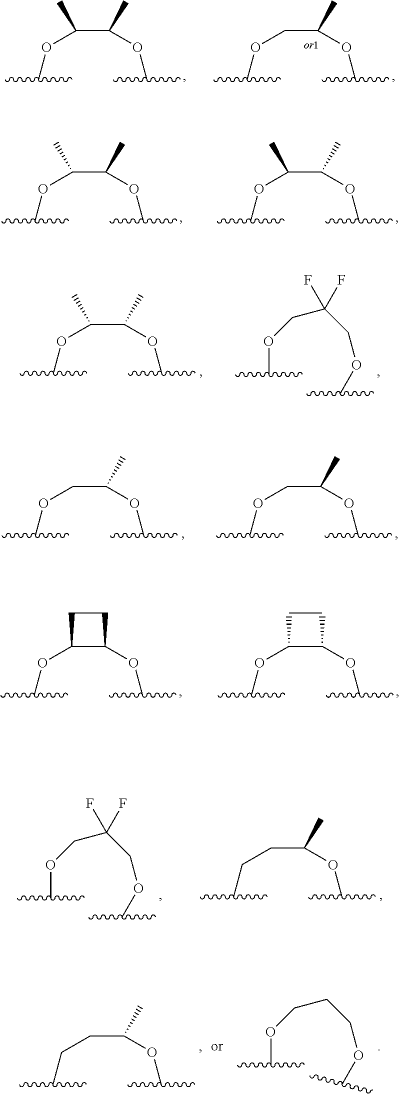

In some embodiments, L₁, L₂, L₃, L₄, and L₅ form a moiety as depicted in the compounds of Table 1, below.

As defined generally above, R³ is —C(O)-L₆, —S(O)₂-L₆, an optionally substituted C₁₋₆ aliphatic, an optionally substituted C₁₋₆ aliphatic substituted with

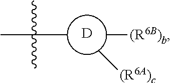

an optionally substituted 5-6 membered monocyclic heteroaromatic ring having 1-4 heteroatoms independently selected from nitrogen, oxygen, and sulfur, or

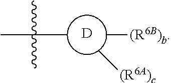

In some embodiments, R³ is —C(O)-L₆. In some embodiments, R³ is —S(O)₂-L₆. In some embodiments, R³ is an optionally substituted C₁₋₆ aliphatic. In some embodiments, R³ is an optionally substituted C₁₋₆ aliphatic substituted with

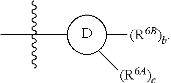

In some embodiments R³ is an optionally substituted 5-6 membered monocyclic heteroaromatic ring having 1-4 heteroatoms independently selected from nitrogen, oxygen, and sulfur. In some embodiments, R³ is

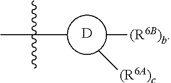

In some embodiments, R³ is

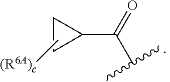

In some embodiments, L₁, L₂, L₃, L₄, and L₅ form

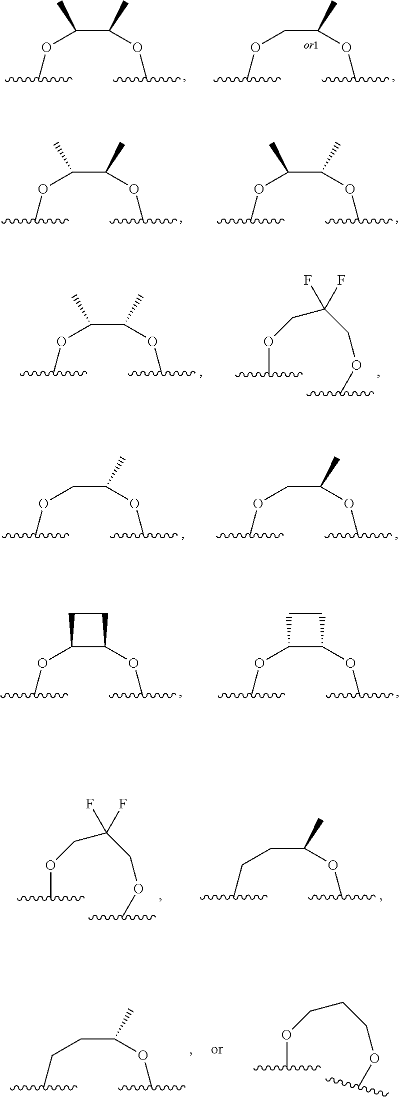

### and R³ is

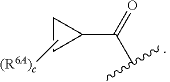

In some embodiments, R³ is as depicted in the compounds of Table 1, below.

As defined generally above, L₆ is

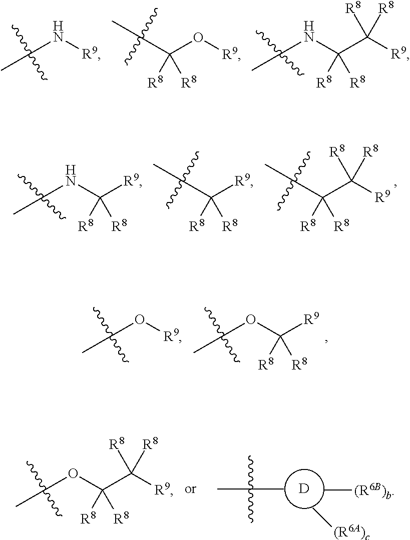

In some embodiments, L₆ is

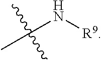

In some embodiments, L₆ is

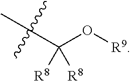

In some embodiments, L₆ is

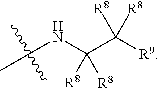

In some embodiments, L₆ is

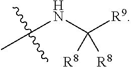

In some embodiments, L₆ is

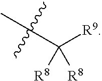

In some embodiments, L₆ is

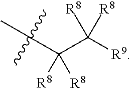

In some embodiments, L₆ is

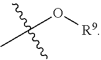

In some embodiments, L₆ is

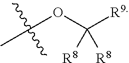

In some embodiments, L₆ is

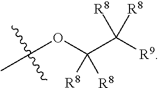

In some embodiments, L₆ is

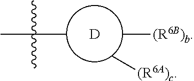

In some embodiments, L₆ is

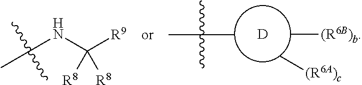

In some embodiments, L₆ is methyl,

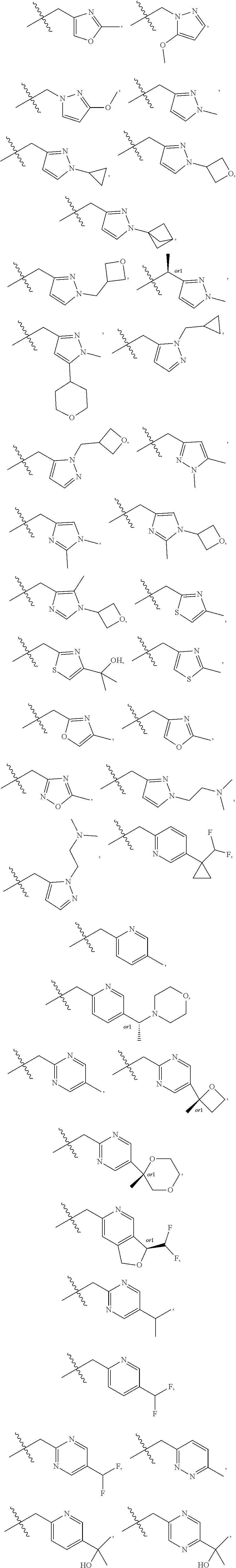

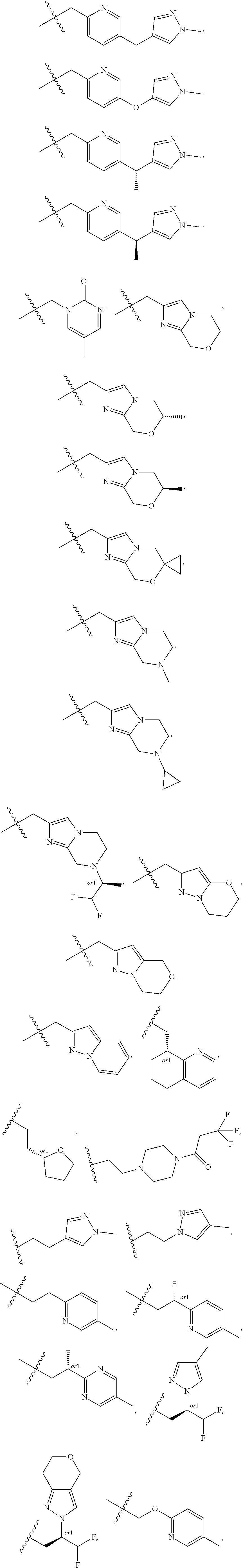

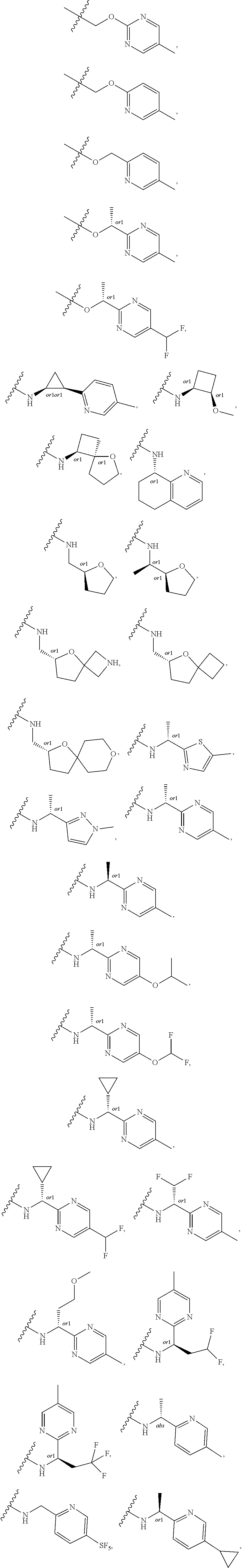

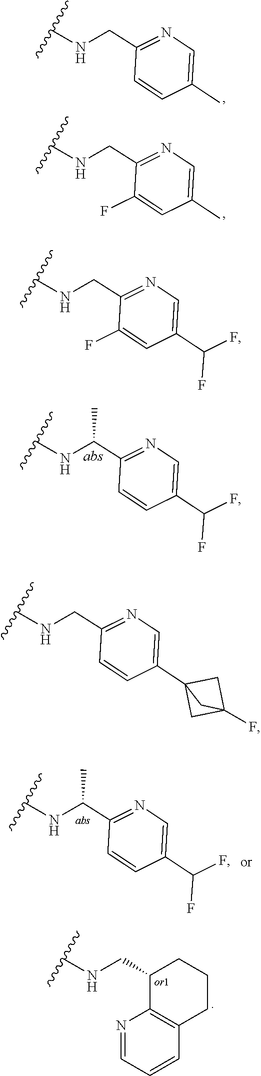

In some embodiments, L₆ is methyl.

In some embodiments, L₆ is

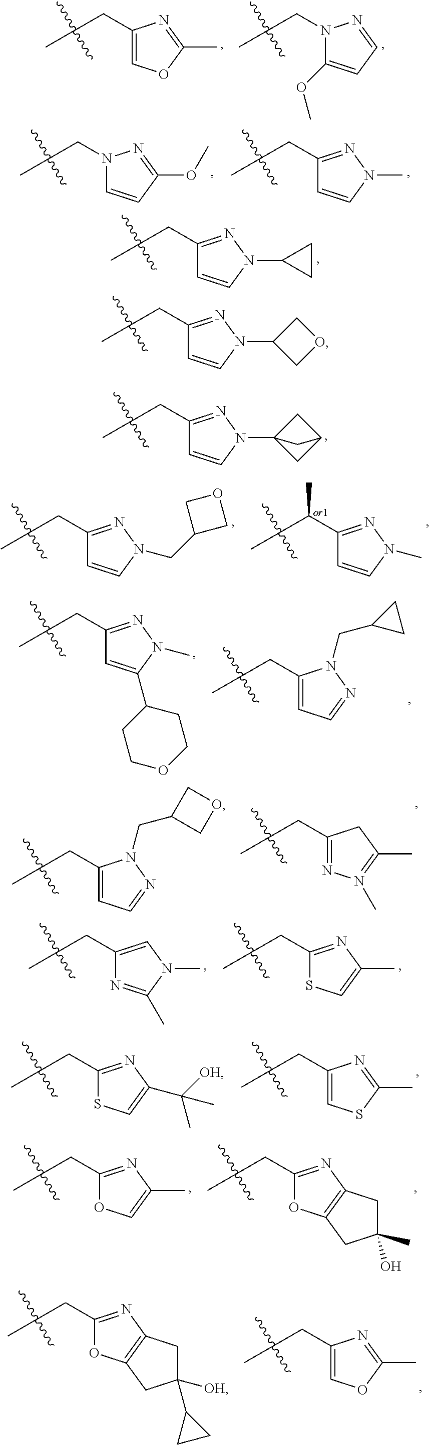

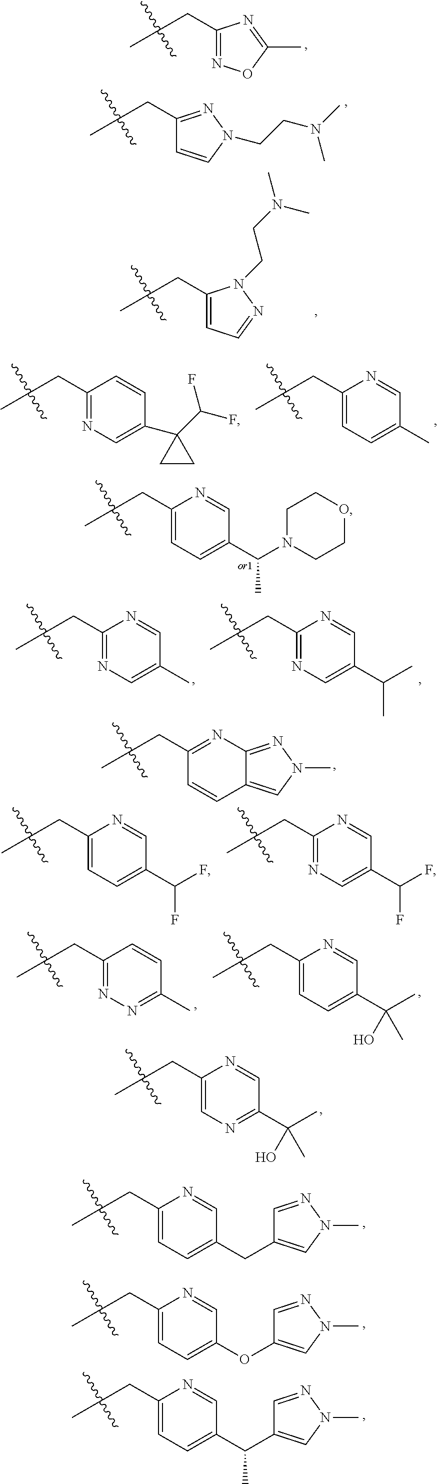

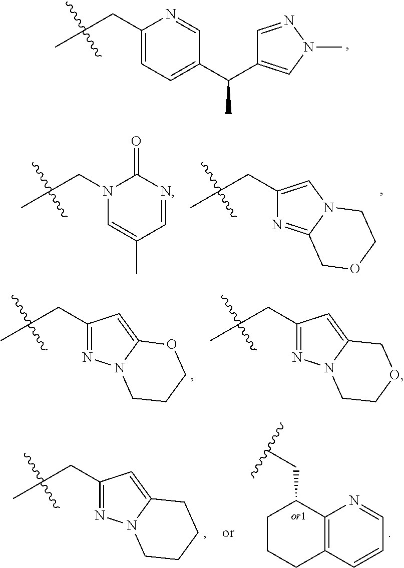

In some embodiments, L₆ is

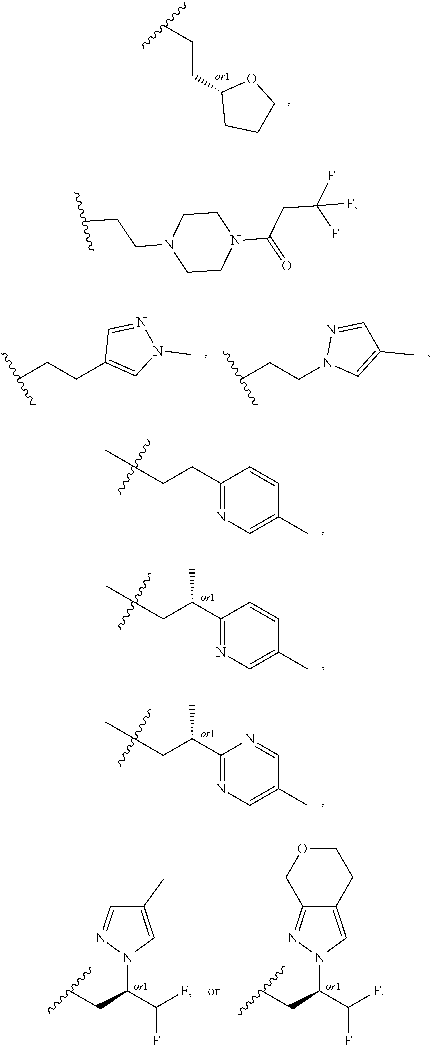

In some embodiments, L₆ is

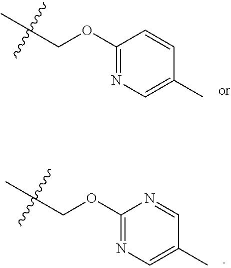

In some embodiments, L₆ is

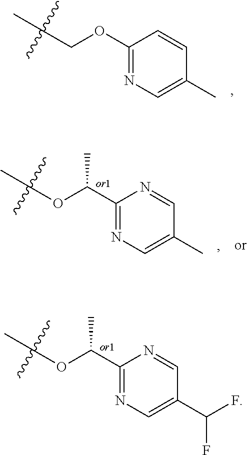

In some embodiments, L₆ is

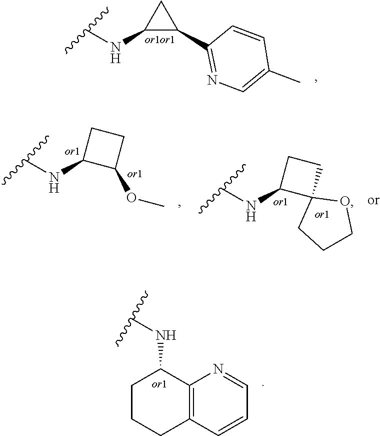

In some embodiments, L₆ is

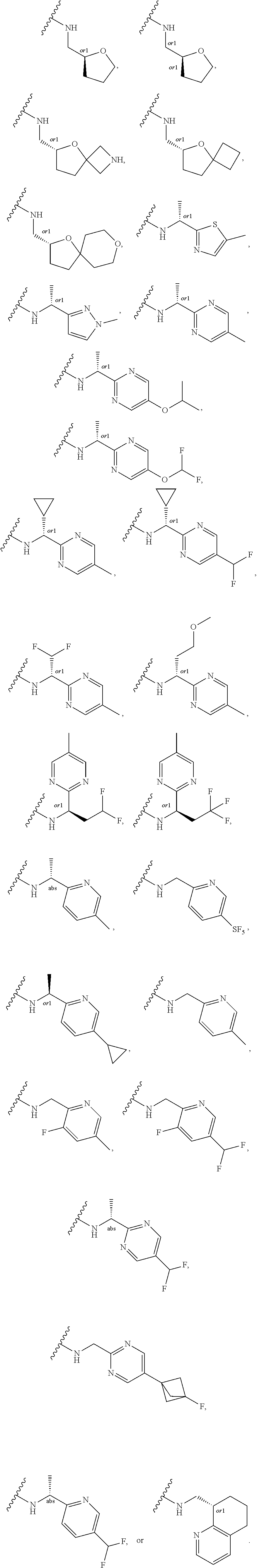

In some embodiments, L₆ is

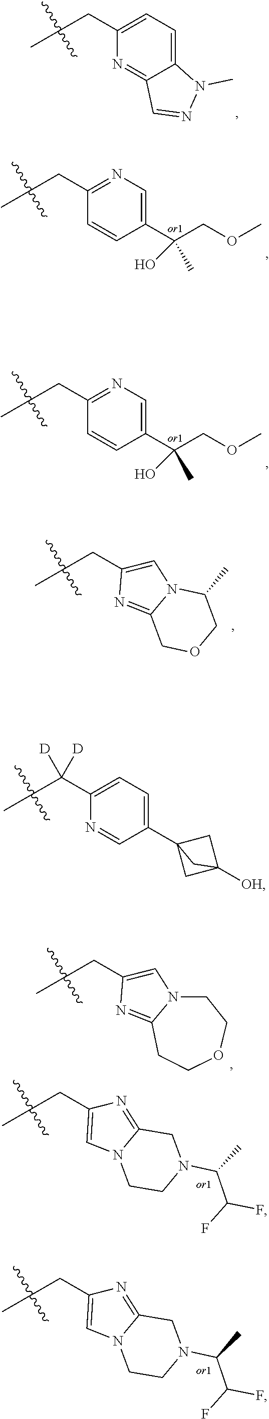

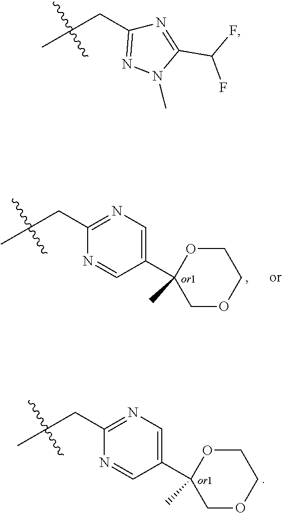 [SMILES: *Cc1nc(C(F)F)n(C)n1 ; *Cc1ncc([C@@]2(C)COCCO2)cn1 ; *Cc1ncc([C@]2(C)COCCO2)cn1]

In some embodiments, L₆ is as depicted in the compounds of Table 1, below.

As defined generally above, each R^{1a}, R^{1b}, R^{2a}, R^{2b}, R^{3a}, R^{3b}, R^{4a}, R^{4b}, R^{5a}, and R^{5b}, are independently selected from the group consisting of hydrogen, halogen, hydroxyl, N(R¹)₂, C₁₋₃ alkyl, —CN, C₁₋₃ alkoxy, C₀₋₃alkyl-C₃-C₆cycloalkyl, C₀₋₃alkyl-C₃-C₆heterocycloalkyl, and hydroxy-C₁₋₃ alkyl, and halo-C₁₋₃ alkyl, or
R^{1a} and R^{1b}, R^{2a} and R^{2b}, R^{3a} and R^{3b}, R^{4a} and R^{4b}, or R^{5a} and R^{5b}, and the carbon atom to which they are attached may combine to form:
   a) a 3-7 membered saturated or partially unsaturated bivalent monocyclic carbocyclylene or 4-7 membered saturated or partially unsaturated bivalent heterocyclylene ring (having 1-4 heteroatoms independently selected from nitrogen, oxygen, and sulfur, and 0 or 1 nitrogen atoms in addition to the 1-4 heteroatoms)—substituted with y instances of R^{B}, or
   b) a 4-12 membered saturated or partially unsaturated bivalent bicyclic ring system that is fused, bridged, or spirocyclic selected from carbocyclylene or heterocyclylene (having 1-4 heteroatoms independently selected from nitrogen, oxygen, and sulfur)—substituted with y instances of R^{B}, or
R^{1a} and R^{2a}, R^{1a} and R^{3a}, R^{2a} and R^{3a}, R^{2a} and R^{4a}, R^{3a} and R^{4a}, R^{3a} and R^{5a}, or R^{4a} and R^{5a}, and the atoms to which they are connected may combine to form:
   a) a 3-7 membered saturated or partially unsaturated bivalent monocyclic carbocyclylene or a 4-7 membered saturated or partially unsaturated bivalent heterocyclylene ring (having 1-4 heteroatoms independently selected from nitrogen, oxygen, and sulfur, and 0 or 1 nitrogen atoms in addition to the 1-4 heteroatoms)—substituted with y instances of R^{B}, or
   b) a 4-12 membered saturated or partially unsaturated bivalent bicyclic ring system that is fused, bridged, or spirocyclic selected from carbocyclylene or heterocyclylene (having 1-4 heteroatoms independently selected from nitrogen, oxygen, and sulfur)—substituted with y instances of R^{B}.

In some embodiments, each R^{1a}, R^{1b}, R^{2a}, R^{2b}, R^{3a}, R^{3b}, R^{4a}, R^{4b}, R^{5a}, and R^{5b}, are independently selected from the group consisting of hydrogen, halogen, hydroxyl, N(R¹)₂, C₁₋₃ alkyl, —CN, C₁₋₃ alkoxy, C₀₋₃ alkylC₃-C₆ cycloalkyl, C₀₋₃ alkylC₃-C₆ heterocycloalkyl, and C₁₋₃ hydroxyalkyl, and C₁₋₃ haloalkyl.

In some embodiments, R^{1a} is hydrogen. In some embodiments, R^{1a} is halogen. In some embodiments, R^{1a} is —CN. In some embodiments, R^{1a} is N(R¹)₂. In some embodiments, R^{1a} is hydroxyl. In some embodiments, R^{1a} is C₁₋₃ alkyl. In some embodiments, R^{1a} is C₁₋₃ alkoxy. In some embodiments, R^{1a} is C₀₋₃ alkylC₃-C₆ cycloalkyl. In some embodiments, R^{1a} is C₀₋₃ alkylC₃-C₆ heterocycloalkyl. In some embodiments, R^{1a} is C₁₋₃ hydroxyalkyl. In some embodiments, Ria is C₁₋₃ haloalkyl.

In some embodiments, R^{1b} is hydrogen. In some embodiments, R^{1b} is halogen. In some embodiments, R^{1b} is —CN. In some embodiments, R^{1b} is N(R¹)₂. In some embodiments, R^{1b} is hydroxyl. In some embodiments, R^{1b} is C₁₋₃ alkyl. In some embodiments, R^{1b} is C₁₋₃ alkoxy. In some embodiments, R^{1b} is C₀₋₃ alkylC₃-C₆ cycloalkyl. In some embodiments, R^{1b} is C₀₋₃ alkylC₃-C₆ heterocycloalkyl. In some embodiments, R^{1b} is C₁₋₃ hydroxyalkyl. In some embodiments, R^{1b} is C₁₋₃ haloalkyl.

In some embodiments, R^{2a} is hydrogen. In some embodiments, R^{2a} is halogen. In some embodiments, R^{2a} is —CN. In some embodiments, R^{2a} is N(R¹)₂. In some embodiments, R^{2a} is hydroxyl. In some embodiments, R^{2a} is C₁₋₃ alkyl. In some embodiments, R^{2a} is C₁₋₃ alkoxy. In some embodiments, R^{2a} is C₀₋₃ alkylC₃-C₆ cycloalkyl. In some embodiments, R^{2a} is C₀₋₃ alkylC₃-C₆ heterocycloalkyl. In some embodiments, R^{2a} is C₁₋₃ hydroxyalkyl. In some embodiments, R^{2a} is C₁₋₃ haloalkyl.

In some embodiments, R^{2b} is hydrogen. In some embodiments, R^{2b} is halogen. In some embodiments, R^{2b} is —CN. In some embodiments, R^{2b} is N(R¹)₂. In some embodiments, R^{2b} is hydroxyl. In some embodiments, R^{2b} is C₁₋₃ alkyl. In some embodiments, R^{2b} is C₁₋₃ alkoxy. In some embodiments, R^{2b} is CO₃ alkylC₃-C₆ cycloalkyl. In some embodiments, R^{2b} is CO₃ alkylC₃-C₆ heterocycloalkyl. In some embodiments, R^{2b} is C₁₋₃ hydroxyalkyl. In some embodiments, R^{2b} is C₁₋₃ haloalkyl.

In some embodiments, R^{3a} is hydrogen. In some embodiments, R^{3a} is halogen. In some embodiments, R^{3a} is —CN. In some embodiments, R^{3a} is N(R¹)₂. In some embodiments, R^{3a} is hydroxyl. In some embodiments, R^{2a} is C₁₋₃ alkyl. In some embodiments, R^{3a} is C₁₋₃ alkoxy. In some embodiments, R^{3a} is CO₃ alkylC₃-C₆ cycloalkyl. In some embodiments, R^{3a} is CO₃ alkylC₃-C₆ heterocycloalkyl. In some embodiments, R^{3a} is C₁₋₃ hydroxyalkyl. In some embodiments, R^{3a} is C₁₋₃ haloalkyl.

In some embodiments, R^{3b} is hydrogen. In some embodiments, R^{3b} is halogen. In some embodiments, R^{3b} is —CN. In some embodiments, R^{3b} is N(R¹)₂. In some embodiments, R^{3b} is hydroxyl. In some embodiments, R^{3b} is C₁₋₃ alkyl. In some embodiments, R^{3b} is C₁₋₃ alkoxy. In some embodiments, R^{3b} is CO₃ alkylC₃-C₆ cycloalkyl. In some embodiments, R^{3b} is CO₃ alkylC₃-C₆ heterocycloalkyl. In some embodiments, R^{3b} is C₁₋₃ hydroxyalkyl. In some embodiments, R^{3b} is C₁₋₃ haloalkyl.

In some embodiments, R^{4a} is hydrogen. In some embodiments, R^{4a} is halogen. In some embodiments, R^{4a} is —CN. In some embodiments, R^{4a} is N(R¹)₂. In some embodiments, R^{4a} is hydroxyl. In some embodiments, R^{4a} is C₁₋₃ alkyl. In some embodiments, R^{4a} is C₁₋₃ alkoxy. In some embodiments, R^{4a} is CO₃ alkylC₃-C₆ cycloalkyl. In some embodiments, R^{4a} is CO₃ alkylC₃-C₆ heterocycloalkyl. In some embodiments, R^{4a} is C₁₋₃ hydroxyalkyl. In some embodiments, R^{4a} is C₁₋₃ haloalkyl.

In some embodiments, R^{4b} is hydrogen. In some embodiments, R^{4b} is halogen. In some embodiments, R^{4b} is —CN. In some embodiments, R^{4b} is N(R¹)₂. In some embodiments, R^{4b} is hydroxyl. In some embodiments, R^{4b} is C₁₋₃ alkyl. In some embodiments, R^{4b} is C₁₋₃ alkoxy. In some embodiments, R^{4b} is CO₃ alkylC₃-C₆ cycloalkyl. In some embodiments, R^{4b} is C₀₋₃ alkylC₃-C₆ heterocycloalkyl. In some embodiments, R^{4b} is C₁₋₃ hydroxyalkyl. In some embodiments, R^{4b} is C₁₋₃ haloalkyl.

In some embodiments, R^{5a} is hydrogen. In some embodiments, R^{5a} is halogen. In some embodiments, R^{5a} is —CN. In some embodiments, R^{5a} is N(R¹)₂. In some embodiments, R^{5a} is hydroxyl. In some embodiments, R^{5a} is C₁₋₃ alkyl. In some embodiments, R^{5a} is C₁₋₃ alkoxy. In some embodiments, R^{5a} is CO₃ alkylC₃-C₆ cycloalkyl. In some embodiments, R^{5a} is C₀₋₃ alkylC₃-C₆ heterocycloalkyl. In some embodiments, R^{5a} is C₁₋₃ hydroxyalkyl. In some embodiments, R⁵, is C₁₋₃ haloalkyl.

In some embodiments, R^{5b} is hydrogen. In some embodiments, R^{5b} is halogen. In some embodiments, R^{5b} is —CN. In some embodiments, R^{5b} is N(R¹)₂. In some embodiments, R^{5b} is hydroxyl. In some embodiments, R^{5b} is C₁₋₃ alkyl. In some embodiments, R^{5b} is C₁₋₃ alkoxy. In some embodiments, R^{5b} is CO₃ alkylC₃-C₆ cycloalkyl. In some embodiments, R^{5b} is CO₃ alkylC₃-C₆ heterocycloalkyl. In some embodiments, R^{5b} is C₁₋₃ hydroxyalkyl. In some embodiments, R^{5b} is C₁₋₃ haloalkyl.

In some embodiments, each R^{1a}, R^{1b}, R^{2a}, R^{2a}, R^{3a}, R^{3b}, R^{4a}, R^{4b}, R^{5a}, and R^{5b}, are independently as depicted in the compounds of Table 1, below.

As generally defined above, each R is independently hydrogen, —C(O)N(CH₃)₂, —C(O)₂CH₃, —C(O)₂C(CH₃)₃, —C(O)₂CH(CH₃)₂, —S(O)₂CH₃, an optionally substituted C₁₋₆ aliphatic group, an optionally substituted cyclic group, or an optionally substituted C₁₋₆ alkyl-cyclic group, wherein the cyclic group is selected from:
phenyl;
an optionally substituted 3-7 membered saturated or partially unsaturated carbocyclic ring;
an optionally substituted 3-7 membered saturated or partially unsaturated heterocyclic ring having 1-2 heteroatoms independently selected from nitrogen, oxygen, and sulfur;
an optionally substituted 5-6 membered heteroaryl ring having 1-4 heteroatoms independently selected from nitrogen, oxygen, and sulfur;
an optionally substituted 8-10 membered bicyclic heteroaryl ring having 1-4 heteroatoms independently selected from nitrogen, oxygen, and sulfur;
an optionally substituted 7-12 membered saturated or partially unsaturated bicyclic heterocyclic ring having 1-4 heteroatoms independently selected from nitrogen, oxygen, and sulfur;
an optionally substituted 5-8 membered saturated or partially unsaturated bridged bicyclic ring having 0-3 heteroatoms independently selected from nitrogen, oxygen, and sulfur;
an optionally substituted 6-10 membered saturated or partially unsaturated spirocyclic ring having 0-3 heteroatoms independently selected from nitrogen, oxygen, and sulfur;
and optionally substituted 6-11 membered saturated or partially unsaturated bicyclic ring having 0-2 heteroatoms independently selected from nitrogen, oxygen, and sulfur; and/or
two R groups on the same atom are taken together with the same atom to form a cyclic group selected from:
an optionally substituted 4-7 membered saturated or partially unsaturated carbocyclyl;
an optionally substituted 4-7 membered saturated or partially unsaturated heterocyclyl having 1-3 heteroatoms independently selected from nitrogen, oxygen, and sulfur; and
an optionally substituted 5-6 membered heteroaryl ring having 1-3 heteroatoms independently selected from nitrogen, oxygen, and sulfur, wherein the optionally substituted R moiety is substituted with 0, 1, 2, 3, or 4 R^{Sub};
each R^{Sub} is independently selected from C₁₋₆ aliphatic group substituted with 0, 1, 2, or 3 groups independently selected from halogen, —CN, —N(R^{N})₂, and —O R^{N}; halogen; —CN; —NO₂; —OR^{N}; —SR^{N}; —N(R^{N})₂; —S(O)₂R^{N}; —S(O)₂N(R^{N})₂; —S(O)R^{N}; —S(O)N(R^{N})R₂; —C(O)R^{N}; —C(O)OR^{N}; —C(O)N(R^{N})₂; —C(O)N(R^{N})OR^{N}; —OC(O)R^{N}; —OC(O)N(R^{N})₂; —N(R^{N})C(O)OR^{N}; —N(R^{N})C(O)R^{N}; —N(R^{N})C(O)N(R^{N})₂; —N(R^{N})C(NRN)N(R^{N})₂; —N(R^{N})S(O)₂N(R^{N})₂; and —N(R^{N})S(O)₂R^{N}; and each R^{N} is independently selected from H and C₁₋₃ alkyl.

In some embodiments, each R is independently hydrogen, or an optionally substituted group selected from C₁₋₆ aliphatic; phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; and a 6-11 membered saturated or partially unsaturated bicyclic ring having 0-2 heteroatoms independently selected from nitrogen, oxygen, and sulfur; or two R groups on the same atom are optionally taken together with the atom to form an optionally substituted 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur.

In some embodiments, R is hydrogen. In some embodiments, R is an optionally substituted C₁₋₆ aliphatic. In some embodiments, R is an optionally substituted phenyl. In some embodiments, R is an optionally substituted naphthyl. In some embodiments, R is an optionally substituted 3-7 membered saturated or partially unsaturated monocyclic carbocyclic ring. In some embodiments, R is an optionally substituted 3-7 membered saturated or partially unsaturated monocyclic heterocyclic ring having 1-2 heteroatoms independently selected from nitrogen, oxygen, and sulfur. In some embodiments, R is an optionally substituted 5-6 membered monocyclic heteroaryl ring having 1-4 heteroatoms independently selected from nitrogen, oxygen, and sulfur. In some embodiments, R is an optionally substituted 8-10 membered bicyclic heteroaryl ring having 1-4 heteroatoms independently selected from nitrogen, oxygen, and sulfur. In some embodiments, R is an optionally substituted 7-12 membered saturated or partially unsaturated bicyclic heterocyclic ring having 1-4 heteroatoms independently selected from nitrogen, oxygen, and sulfur. In some embodiments, R is an optionally substituted 5-8 membered saturated or partially unsaturated bridged bicyclic ring having 0-3 heteroatoms independently selected from nitrogen, oxygen, and sulfur. In some embodiments, R is an optionally substituted 6-10 membered saturated or partially unsaturated spirocyclic ring having 0-3 heteroatoms independently selected from nitrogen, oxygen, and sulfur. In some embodiments, R is an optionally substituted 6-11 membered saturated or partially unsaturated bicyclic ring having 0-2 heteroatoms independently selected from nitrogen, oxygen, and sulfur. In some embodiments, two R groups on the same atom are taken together with the atom to form an optionally substituted 3-7 membered saturated or partially unsaturated monocyclic carbocyclic ring. In some embodiments, two R groups on the same atom are taken together with the atom to form an optionally substituted 3-7 membered saturated or partially unsaturated monocyclic heterocyclic ring having 1-2 heteroatoms independently selected from nitrogen, oxygen, and sulfur. In some embodiments, two R groups on the same atom are optionally taken together with the atom to form an optionally substituted 5-6 membered heteroaryl ring having 1-3 heteroatoms independently selected from nitrogen, oxygen, and sulfur.

In some embodiments, each R is as depicted in the compounds of Table 1, below.

In some embodiments, the compound of formula I is a compound of formulae II-a, II-b, II-c, II-d, or II-e:

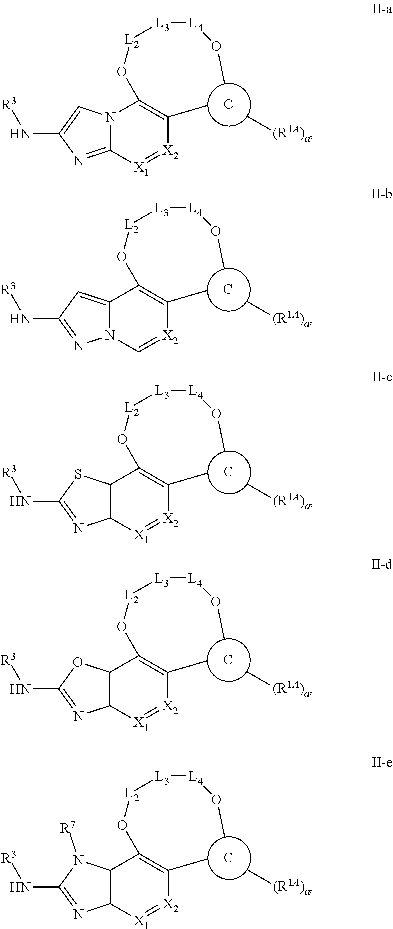

or a pharmaceutically acceptable salt thereof, wherein:
X₁ and X₂ are independently selected from CH, N, CF, CCl, CCH₃; and
Ring C is a 5-membered monocyclic heteroaryl having 1-3 heteroatoms independently selected from nitrogen, oxygen, and sulfur.

In some embodiments, the present invention provides a compound formula I is a compound of formulae II-a, II-b, II-c, II-d, or II-e or a pharmaceutically acceptable salt thereof, wherein:
X₁ and X₂ and are independently selected from N, CF, CCl, CCH₃ and CH; and
Ring C is a 6-membered monocyclic heteroaryl having 1-3 heteroatoms independently selected from nitrogen, oxygen, and sulfur.

In some embodiments, the present invention provides a compound formula I is a compound of formulae II-a, II-b, II-c, II-d, or II-e or a pharmaceutically acceptable salt thereof, wherein:
X₁ and X₂ and are independently selected from N, CF, CCl, CCH₃ and CH; and
Ring C is an 8-10 membered bicyclic heteroaryl having 1-4 heteroatoms independently selected from nitrogen, oxygen, and sulfur.

In some embodiments, the present invention provides a compound formula I is a compound of formulae II-a, II-b, II-c, II-d, or II-e, or a pharmaceutically acceptable salt thereof, wherein:
X₁ and X₂ and are independently selected from N, CF, CCl, CCH₃ and CH; and
Ring C is a 7-12 membered saturated or partially unsaturated bicyclic heterocyclyl having 1-4 heteroatoms independently selected from nitrogen, oxygen, and sulfur.

In some embodiments, the compound of formula I is a compound of formulae II-a, II-b, II-c, II-d, or II-e:

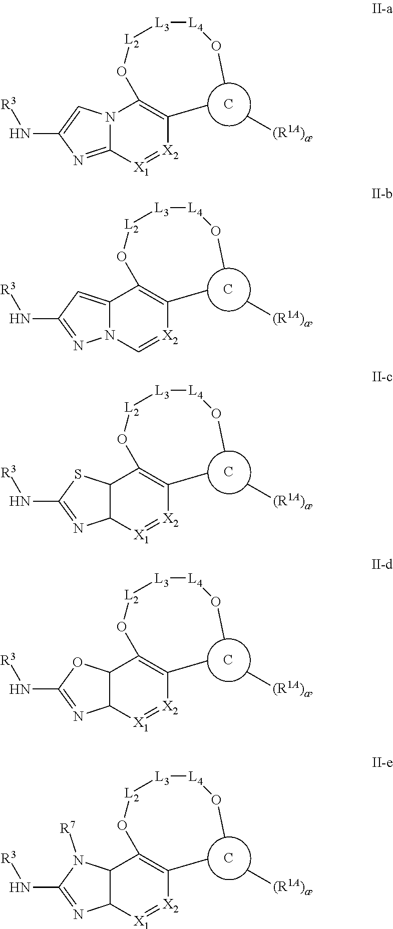

or a pharmaceutically acceptable salt thereof, wherein:
X₁ and X₂ are independently selected from CH, N, CF, CCl, CCH₃.

In some embodiments, the compound of formula I is a compound of formulae III-a, III-b, III-c, III-d, or III-e:

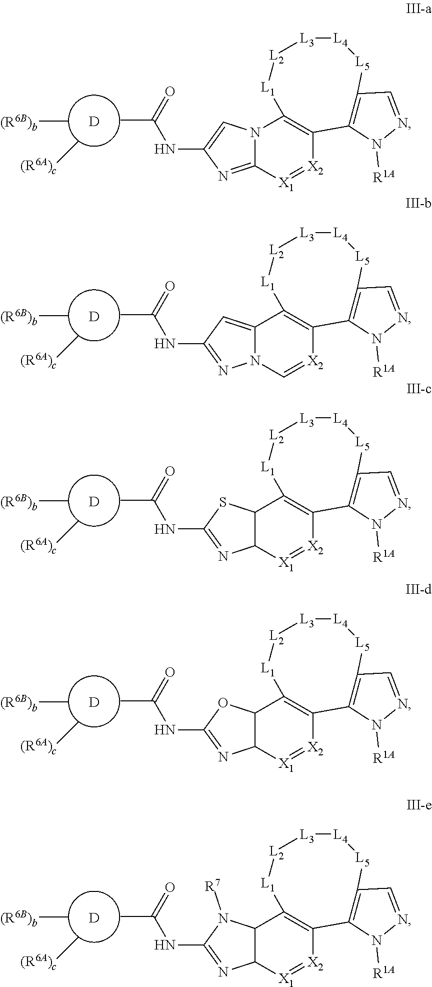

or a pharmaceutically acceptable salt thereof, wherein:
X₁ and X_{z} are independently selected from CH, N, CF, CCl, and CCH₃.

In some embodiments, the compound of formula I is a compound of formulae IV-a, IV-b, IV-c, IV-d, or VI-e:

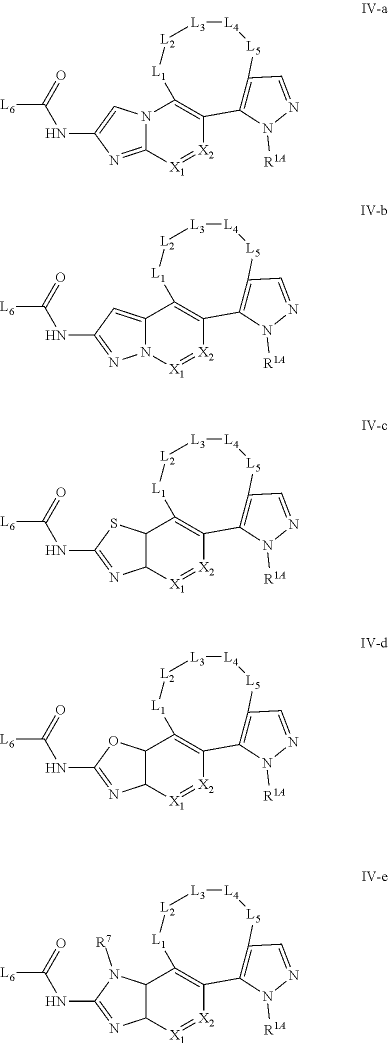

or a pharmaceutically acceptable salt thereof, wherein:
X₁ and X_{z} are independently selected from CH, N, CF, CCl, CCH₃; and L₆ is selected from the group consisting of

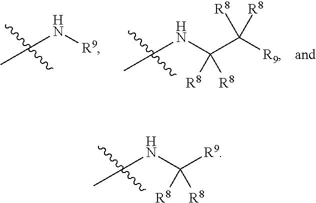

In some embodiments, the compound of formula I is a compound of formulae V-a, V-b, V-c, V-d, or V-e:

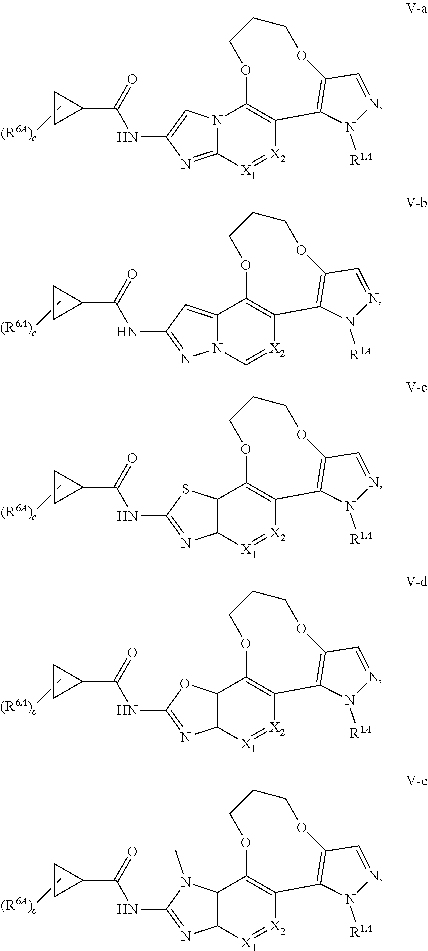

or a pharmaceutically acceptable salt thereof, wherein:
X₁ and X₂ are independently selected from CH, N, CF, CCl, and CCH₃.

In some embodiments, the compound of formula I is a compound of formulae VI-a or VI-b:

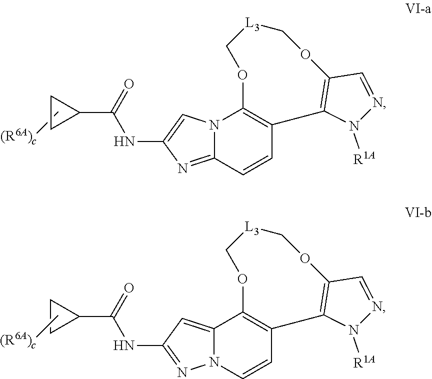

or a pharmaceutically acceptable salt thereof, wherein:
L₃ is independently selected from CH₂ and CF₂.

In some embodiments, the compound of formula I is a compound of formulae VII-a, VII-b, VII-c, VII-d, or VII-e:

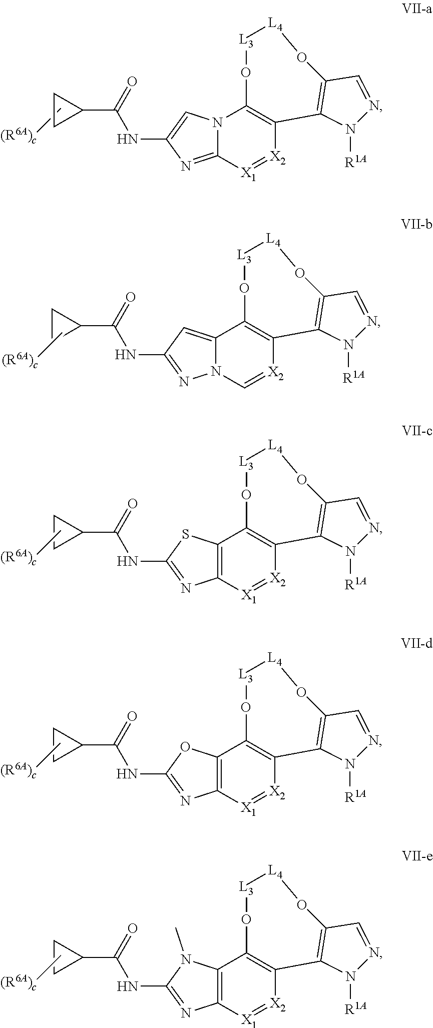

or a pharmaceutically acceptable salt thereof, wherein:
L₃ is independently selected from CH₂ and CHCH₃; and L₄ is independently selected from CH₂ and CHCH₃.

In some embodiments, the compound of formula I is a compound of formulae VII-a or VII-b:

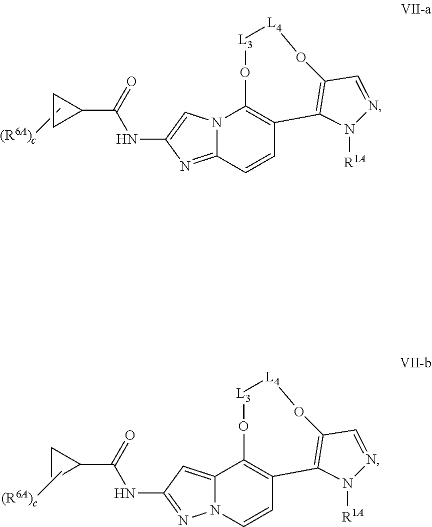

or a pharmaceutically acceptable salt thereof, wherein:
L₃ is independently selected from CH₂ and CHCH₃; and L₄ is independently selected from CH₂ and CHCH₃.

In some embodiments, the compound of formula I is a compound of formulae VIII-a, or VIII-b:

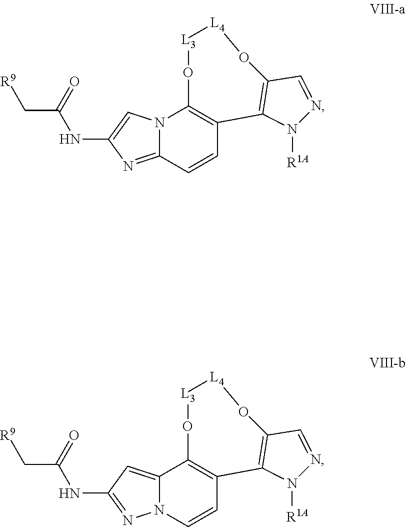

or a pharmaceutically acceptable salt thereof, wherein:
L₃ is independently selected from CH₂ and CHCH₃; and L₄ is independently selected from CH₂ and CHCH₃.

In some embodiments, the compound of formula I is a compound of formulae IX-a or IX-b:

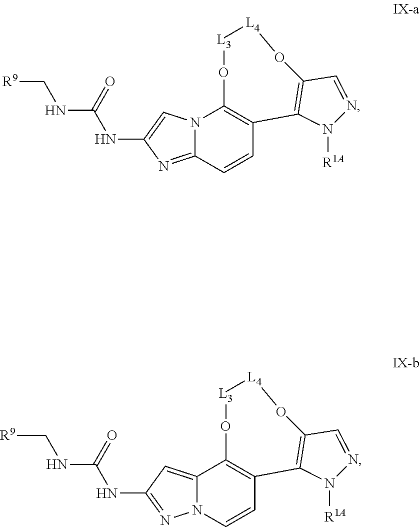

or a pharmaceutically acceptable salt thereof, wherein:
L₃ is independently selected from CH₂ and CHCH₃; and L₄ is independently selected from CH₂ and CHCH₃.

In some embodiments, the compound of formula X is a compound of formulae X-a, X-b:

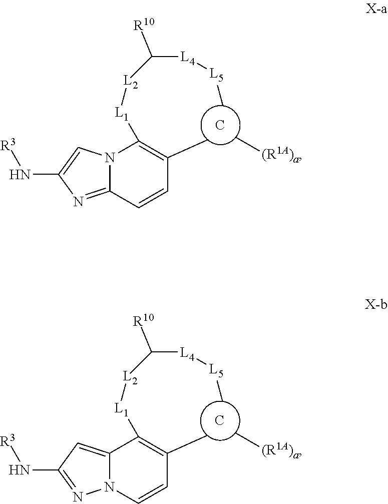

or a pharmaceutically acceptable salt thereof, wherein:
L₁ is selected from a bond, O, S and CH₂; L₅ is selected from a bond, O, S and CH₂; and R¹⁰ is independently selected from a H, —S—C₁₋₃ alkyl, —CH₂S—C₁₋₃ alkyl.

Exemplary compounds of the invention are set forth in Table 1, or a pharmaceutically acceptable salt thereof. Table 1 identifies compounds by their IUPAC name and Table 2 lists the same compounds and shows their chemical structure. The chemical names in the present application are generated from the corresponding structures using either CHEMDRAW, or CHEMAXON. In some instances, chemical names generated from the structures may give a different structure when using the “Convert Name to Structure” function in CHEMDRAW. In the event of any discrepancy between Table 1's name for a compound and Table 2's structure for that same compound, Table 2's compound structures will dominate and identify the compound corresponding to each respective compound number (I-#) in Table 1.

TABLE 1
Exemplary Compounds

| I-# | IUPAC name |
|---|---|
| I-1 | (1S,2R)-N-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}-2-(5-methylpyridin-2-yl)cyclopropane-1-carboxamide |
| I-2 | (1S,2R)-2-[5-(difluoromethyl)pyridin-2-yl]-N-{3-methyl-7,11-dioxa-3,4,16,17- |
| | tetraazatetracyclo[10.7.0.0\hat{ }{2,6}.0\hat{ }{13,17}]nonadeca-1(12),2(6),4,13,15,18-hexaen- |
| | 15-yl}cyclopropane-1-carboxamide |
| I-3 | N-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}cyclopropanecarboxamide |
| I-4 | (1S,2R)-N-{3-methyl-7,11-dioxa-3,4,16,17- |
| | tetraazatetracyclo[10.7.0.0\hat{ }{2,6}.0\hat{ }{13,17}]nonadeca-1(12),2(6),4,13,15,18-hexaen- |
| | 15-yl}-2-(5-methylpyridin-2-yl)cyclopropane-1-carboxamide |
| I-5 | (1S,2R)-N-[(9R)-3,9-dimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]-2-(5-methylpyridin-2-yl)cyclopropane-1-carboxamide |
| I-6 | (1S,2R)-N-[(8R)-3,8-dimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]-2-(5-methylpyridin-2-yl)cyclopropane-1-carboxamide |
| I-7 | (1S,2R)-2-[5-(azetidin-1-yl)pyridin-2-yl]-N-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}cyclopropane-1-carboxamide |
| I-8 | (1S,2R)-2-(5-fluoropyridin-2-yl)-N-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}cyclopropane-1-carboxamide |
| I-9 | (1S,2R)-N-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}-2-(pyridin-2-yl)cyclopropane-1-carboxamide |
| I-10 | N-(2,6-dimethylpyrimidin-4-yl)-3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-amine |
| I-11 | 3-methyl-N-(6-methylpyridazin-3-yl)-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-amine |
| I-12 | 3-methyl-1-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}urea |
| I-13 | (1S,2R)-N-[(9S)-3,9-dimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]-2-(5-methylpyridin-2-yl)cyclopropane-1-carboxamide |
| I-14 | (1S,2R)-N-[(8S)-3,8-dimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]-2-(5-methylpyridin-2-yl)cyclopropane-1-carboxamide |
| I-15 | (1S,2R)-N-{3-methyl-10-oxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}-2-(5-methylpyridin-2-yl)cyclopropane-1-carboxamide |
| I-16 | (1S,2R)-2-(4-methoxy-5-methylpyridin-2-yl)-N-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}cyclopropane-1-carboxamide |
| I-17 | (1S,2R)-2-{imidazo[1,2-b]pyridazin-2-yl}-N-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}cyclopropane-1-carboxamide |
| I-18 | (1S,2R)-2-(4,5-dimethylpyridin-2-yl)-N-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}cyclopropane-1-carboxamide |
| I-19 | (1S,2R)-N-{9-hydroxy-3,9-dimethyl-7,11-dioxa-3,4,16,17- |
| | tetraazatetracyclo[10.7.0.0\hat{ }{2,6}.0\hat{ }{13,17}]nonadeca-1(12),2(6),4,13,15,18-hexaen- |
| | 15-yl}-2-(5-methylpyridin-2-yl)cyclopropane-1-carboxamide |
| I-20 | (1S,2R)-2-{5H,6H,8H-imidazo[2,1-c][1,4]oxazin-2-yl}-N-{3-methyl-7,10-dioxa- |
| | 3,4,15,16-tetraazatetracyclo[9.7.0.0\hat{ }{2,6}.0\hat{ }{12,16}]octadeca-1(11),2(6),4,12,14,17- |
| | hexaen-14-yl}cyclopropane-1-carboxamide |
| I-21 | (1S,2R)-N-{4-methyl-8,11-dioxa-5,16,17- |
| | triazatetracyclo[10.7.0.0\hat{ }{2,7}.0\hat{ }{13,17}]nonadeca-1(12),2(7),3,5,13,15,18-heptaen- |
| | 15-yl}-2-(5-methylpyridin-2-yl)cyclopropane-1-carboxamide |
| I-22 | (1S,2R)-N-[3-(difluoromethyl)-7,10-dioxa-3,4,15,16,18- |
| | pentaazatetracyclo[9.7.0.0\hat{ }{2,6}.0\hat{ }{12,16}]octadeca-1(11),2(6),4,12,14,17-hexaen- |
| | 14-yl]-2-(5-methylpyridin-2-yl)cyclopropane-1-carboxamide |
| I-23 | (1S,2R)-2-(1-cyclopropyl-1H-1,2,3-triazol-4-yl)-N-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}cyclopropane-1-carboxamide |
| I-24 | (1S,2R)-2-(3-hydroxy-4-methylpyridin-2-yl)-N-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}cyclopropane-1-carboxamide |
| I-25 | (1S,2R)-2-[5-(difluoromethyl)pyridin-2-yl]-N-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}cyclopropane-1-carboxamide |
| I-26 | (1S,2R)-N-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}-2-(4-methylpyridin-2-yl)cyclopropane-1-carboxamide |
| I-27 | (1S,2R)-2-(5-fluoro-4-methylpyridin-2-yl)-N-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}cyclopropane-1-carboxamide |
| I-28 | (1S,2R)-2-{imidazo[1,2-a]pyridin-2-yl}-N-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}cyclopropane-1-carboxamide |
| I-29 | (1S,2R)-N-{3-methyl-7,10-dioxa-3,4,15,16,18- |
| | pentaazatetracyclo[9.7.0.0\hat{ }{2,6}.0\hat{ }{12,16}]octadeca-1(11),2(6),4,12,14,17-hexaen- |
| | 14-yl}-2-(5-methylpyridin-2-yl)cyclopropane-1-carboxamide |
| I-30 | (1S,2R)-2-(2-cyclopropyl-2H-1,2,3,4-tetrazol-5-yl)-N-{3-methyl-7,10-dioxa- |
| | 3,4,15,16-tetraazatetracyclo[9.7.0.0\hat{ }{2,6}.0\hat{ }{12,16}]octadeca-1(11),2(6),4,12,14,17- |
| | hexaen-14-yl}cyclopropane-1-carboxamide |
| I-31 | 2-(5-methyl-2-oxo-1,2-dihydropyrimidin-1-yl)-N-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}acetamide |
| I-32 | N-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}-2-[(5-methylpyridin-2-yl)oxy]acetamide |
| I-33 | 3-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}-1-[(5-methylpyridin-2-yl)methyl]urea |
| I-34 | 3-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}-1-[(5-methylpyrimidin-2-yl)methyl]urea |
| I-35 | 2-(5-methyl-2-oxo-1,2-dihydropyridin-1-yl)-N-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}acetamide |
| I-36 | rel-3-[(1R,2S)-2-methoxycyclobutyl]-1-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}urea (stereoisomer 1) |
| I-37 | rel-3-[(1R,2S)-2-methoxycyclobutyl]-1-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}urea (stereoisomer 2) |
| I-38 | (1S,2R)-2-[5-(fluoromethyl)pyridin-2-yl]-N-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}cyclopropane-1-carboxamide |
| I-39 | (5-methylpyridin-2-yl)methylN-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}carbamate |
| I-40 | (1S,2R)-N-{3-methyl-7,10-dioxa-3,4,12,15,18- |
| | pentaazatetracyclo[9.7.0.0\hat{ }{2,6}.0\hat{ }{12,16}]octadeca-1(11),2(6),4,13,15,17-hexaen- |
| | 14-yl}-2-(5-methylpyridin-2-yl)cyclopropane-1-carboxamide |
| I-41 | (1S,2R)-N-{3,7-dimethyl-8-oxo-10-oxa-3,4,7,15,16- |
| | pentaazatetracyclo[9.7.0.0\hat{ }{2,6}.0\hat{ }{12,16}]octadeca-1(11),2(6),4,12,14,17-hexaen- |
| | 14-yl}-2-(5-methylpyridin-2-yl)cyclopropane-1-carboxamide |
| I-42 | (1S,2R)-N-[(9R*)-9-hydroxy-3,9-dimethyl-7,11-dioxa-3,4,16,17- |
| | tetraazatetracyclo[10.7.0.0\hat{ }{2,6}.0\hat{ }{13,17}]nonadeca-1(12),2(6),4,13,15,18-hexaen- |
| | 15-yl]-2-(5-methylpyridin-2-yl)cyclopropane-1-carboxamide (stereoisomer 1) |
| I-43 | (1S,2R)-N-[(9R*)-9-hydroxy-3,9-dimethyl-7,11-dioxa-3,4,16,17- |
| | tetraazatetracyclo[10.7.0.0\hat{ }{2,6}.0\hat{ }{13,17}]nonadeca-1(12),2(6),4,13,15,18-hexaen- |
| | 15-yl]-2-(5-methylpyridin-2-yl)cyclopropane-1-carboxamide (stereoisomer 2) |
| I-44 | N-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}-2-[(5-methylpyrimidin-2-yl)oxy]acetamide |
| I-45 | (1S,2R)-N-[3-(difluoromethyl)-7,10-dioxa-3,4,12,15,18- |
| | pentaazatetracyclo[9.7.0.0\hat{ }{2,6}.0\hat{ }{12,16}]octadeca-1(11),2(6),4,13,15,17-hexaen- |
| | 14-yl]-2-(5-methylpyridin-2-yl)cyclopropane-1-carboxamide |
| I-46 | (1S,2R)-N-[(8R*)-8-(2-hydroxypropan-2-yl)-3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]-2-(5-methylpyridin-2-yl)cyclopropane-1-carboxamide (stereoisomer 1) |
| I-47 | (1S,2R)-N-[(8R*)-8-(2-hydroxypropan-2-yl)-3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]-2-(5-methylpyridin-2-yl)cyclopropane-1-carboxamide (stereoisomer 2) |
| I-48 | (1S,2R)-N-{3-methyl-10-oxa-4,6,15,16- |
| | tetraazatetracyclo[9.7.0.0\hat{ }{2,6}.0\hat{ }{12,16}]octadeca-1(11),2,4,12,14,17-hexaen-14- |
| | yl}-2-(5-methylpyridin-2-yl)cyclopropane-1-carboxamide |
| I-49 | (1S,2R)-N-{3′-methyl-7′,11′-dioxa-3′,4′,16′,17′-tetraazaspiro[oxetane-3,9′- |
| | tetracyclo[10.7.0.0\hat{ }{2,6}.0\hat{ }{13,17}]nonadecane]-1′(12′),2′(6′),4′,13′,15′,18′-hexaen- |
| | 15′-yl}-2-(5-methylpyridin-2-yl)cyclopropane-1-carboxamide |
| I-50 | (1S,2R)-N-{3-methyl-10-oxa-5,6,15,16- |
| | tetraazatetracyclo[9.7.0.0\hat{ }{2,6}.0\hat{ }{12,16}]octadeca-1(11),2,4,12,14,17-hexaen-14- |
| | yl}-2-(5-methylpyridin-2-yl)cyclopropane-1-carboxamide |
| I-51 | (1S,2R)-N-{3-methyl-10-oxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}-2-(5-methylpyrimidin-2-yl)cyclopropane-1-carboxamide |
| I-52 | 2-(5-methoxy-1H-pyrazol-1-yl)-N-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}acetamide |
| I-53 | (1S,2R)-2-{2-methyl-2H-pyrazolo[3,4-c]pyridin-5-yl}-N-{3-methyl-7,10-dioxa- |
| | 3,4,15,16-tetraazatetracyclo[9.7.0.0\hat{ }{2,6}.0\hat{ }{12,16}]octadeca-1(11),2(6),4,12,14,17- |
| | hexaen-14-yl}cyclopropane-1-carboxamide |
| I-54 | (1S,2R)-N-{3-methyl-7,11-dioxa-3,4,16,17- |
| | tetraazatetracyclo[10.7.0.0\hat{ }{2,6}.0\hat{ }{13,17}]nonadeca-1(12),2(6),4,13,15,18-hexaen- |
| | 15-yl}-2-(5-methylpyrimidin-2-yl)cyclopropane-1-carboxamide |
| I-55 | (1S,2R)-N-{3′-methyl-7′,11′-dioxa-3′,4′,16′,17′-tetraazaspiro[cyclopropane-1,9′- |
| | tetracyclo[10.7.0.0\hat{ }{2,6}.0\hat{ }{13,17}]nonadecane]-1′(12′),2′(6′),4′,13′,15′,18′-hexaen- |
| | 15′-yl}-2-(5-methylpyrimidin-2-yl)cyclopropane-1-carboxamide |
| I-56 | (1S,2R)-2-(5-methylpyridin-2-yl)-N-[(1s,18s)-13-methyl-2,17-dioxa-7,8,13,14- |
| | tetraazapentacyclo[16.1.1.0\hat{ }{3,11}.0\hat{ }{4,8}.0\hat{ }{12,16}]icosa-3(11),4,6,9,12(16),14- |
| | hexaen-6-yl]cyclopropane-1-carboxamide |
| I-57 | (1S,2R)-N-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}-2-[5-(2H3)methylpyridin-2-yl]cyclopropane-1-carboxamide |
| I-58 | (1S,2R)-N-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}-2-[5-(trifluoromethyl)pyridin-2-yl]cyclopropane-1-carboxamide |
| I-59 | 2-(3-methoxy-1H-pyrazol-1-yl)-N-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}acetamide |
| I-60 | (1S,2R)-N-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}-2-(5-methylpyrimidin-2-yl)cyclopropane-1-carboxamide |
| I-61 | 3-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}-1-[(1R)-1-(5-methylpyridin-2-yl)ethyl]urea |
| I-62 | (1S,2R)-2-(5-{bicyclo[1.1.1]pentan-1-yl}pyrimidin-2-yl)-N-{3-methyl-7,10-dioxa- |
| | 3,4,15,16-tetraazatetracyclo[9.7.0.0\hat{ }{2,6}.0\hat{ }{12,16}]octadeca-1(11),2(6),4,12,14,17- |
| | hexaen-14-yl}cyclopropane-1-carboxamide |
| I-63 | (1S,2R)-N-[(9R)-3,9-dimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]-2-(5-methylpyrimidin-2-yl)cyclopropane-1-carboxamide |
| I-64 | (1S,2R)-N-[(8R)-3,8-dimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]-2-(5-methylpyrimidin-2-yl)cyclopropane-1-carboxamide |
| I-65 | (1S,2R)-2-(5-cyclopropylpyrimidin-2-yl)-N-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}cyclopropane-1-carboxamide |
| I-66 | (1S,2R)-N-{3,9-dimethyl-10-oxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}-2-(5-methylpyrimidin-2-yl)cyclopropane-1-carboxamide |
| I-67 | (1S,2R)-2-(4-fluoro-5-methylpyridin-2-yl)-N-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}cyclopropane-1-carboxamide |
| I-68 | rel-(3R)-4,4-difluoro-3-(4-methyl-1H-pyrazol-1-yl)-N-{3-methyl-7,10-dioxa- |
| | 3,4,15,16-tetraazatetracyclo[9.7.0.0\hat{ }{2,6}.0\hat{ }{12,16}]octadeca-1(11),2(6),4,12,14,17- |
| | hexaen-14-yl}butanamide (stereoisomer 1) |
| I-69 | rel-(3R)-4,4-difluoro-3-(4-methyl-1H-pyrazol-1-yl)-N-{3-methyl-7,10-dioxa- |
| | 3,4,15,16-tetraazatetracyclo[9.7.0.0\hat{ }{2,6}.0\hat{ }{12,16}]octadeca-1(11),2(6),4,12,14,17- |
| | hexaen-14-yl}butanamide (stereoisomer 2) |
| I-70 | (1S,2R)-N-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}-2-[5-(trifluoromethyl)pyrimidin-2-yl]cyclopropane-1-carboxamide |
| I-71 | (1S,2R)-2-[5-(difluoromethyl)pyridin-2-yl]-N-[(9R)-3,9-dimethyl-7,10-dioxa- |
| | 3,4,15,16-tetraazatetracyclo[9.7.0.0\hat{ }{2,6}.0\hat{ }{12,16}]octadeca-1(11),2(6),4,12,14,17- |
| | hexaen-14-yl]cyclopropane-1-carboxamide |
| I-72 | (1S,2R)-2-(4,5-dimethylpyrimidin-2-yl)-N-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}cyclopropane-1-carboxamide |
| I-73 | (1S,2R)-N-[(10R)-3,10-dimethyl-7,11-dioxa-3,4,16,17- |
| | tetraazatetracyclo[10.7.0.0\hat{ }{2,6}.0\hat{ }{13,17}]nonadeca-1(12),2(6),4,13,15,18-hexaen- |
| | 15-yl]-2-(5-methylpyrimidin-2-yl)cyclopropane-1-carboxamide |
| I-74 | (1S,2R)-N-[(8R)-3,8-dimethyl-7,11-dioxa-3,4,16,17- |
| | tetraazatetracyclo[10.7.0.0\hat{ }{2,6}.0\hat{ }{13,17}]nonadeca-1(12),2(6),4,13,15,18-hexaen- |
| | 15-yl]-2-(5-methylpyrimidin-2-yl)cyclopropane-1-carboxamide |
| I-75 | (1S,2R)-2-[5-(difluoromethyl)pyridin-2-yl]-N-{3-methyl-10-oxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}cyclopropane-1-carboxamide |
| I-76 | (1S,2R)-N-{9,9-difluoro-3-methyl-7,11-dioxa-3,4,16,17- |
| | tetraazatetracyclo[10.7.0.0\hat{ }{2,6}.0\hat{ }{13,17}]nonadeca-1(12),2(6),4,13,15,18-hexaen- |
| | 15-yl}-2-(5-methylpyrimidin-2-yl)cyclopropane-1-carboxamide |
| I-77 | (1S,2R)-2-(5-cyclopropylpyrimidin-2-yl)-N-[(10R)-3,10-dimethyl-7,11-dioxa- |
| | 3,4,16,17-tetraazatetracyclo[10.7.0.0\hat{ }{2,6}.0\hat{ }{13,17}]nonadeca- |
| | 1(12),2(6),4,13,15,18-hexaen-15-yl]cyclopropane-1-carboxamide |
| I-78 | N-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}methanesulfonamide |
| I-79 | (1S,2R)-2-(5-cyclopropylpyrimidin-2-yl)-N-[(8R)-3,8-dimethyl-7,11-dioxa-3,4,16,17- |
| | tetraazatetracyclo[10.7.0.0\hat{ }{2,6}.0\hat{ }{13,17}]nonadeca-1(12),2(6),4,13,15,18-hexaen- |
| | 15-yl]cyclopropane-1-carboxamide |
| I-80 | N-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}cyclopropanesulfonamide |
| I-81 | (1S,2R)-2-[5-(fluoromethyl)pyridin-2-yl]-N-{3-methyl-10-oxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}cyclopropane-1-carboxamide |
| I-82 | (1S,2R)-2-[5-(fluoromethyl)pyridin-2-yl]-N-{3-methyl-7,11-dioxa-3,4,16,17- |
| | tetraazatetracyclo[10.7.0.0\hat{ }{2,6}.0\hat{ }{13,17}]nonadeca-1(12),2(6),4,13,15,18-hexaen- |
| | 15-yl}cyclopropane-1-carboxamide |
| I-83 | 3-[(9R)-3,9-dimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]-1-[(5-methylpyridin-2-yl)methyl]urea |
| I-84 | (1S,2R)-N-{18-fluoro-3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}-2-(5-methylpyridin-2-yl)cyclopropane-1-carboxamide |
| I-85 | 3-{3-methyl-10-oxa-3,4,15,16-tetraazatetracyclo[9.7.0.0\hat{ }{2,6}.0\hat{ }{12,16}]octadeca- |
| | 1(11),2(6),4,12,14,17-hexaen-14-yl}-1-[(5-methylpyridin-2-yl)methyl]urea |
| I-86 | (1S,2R)-N-[(9R*)-3,9-dimethyl-10-oxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]-2-(5-methylpyrimidin-2-yl)cyclopropane-1-carboxamide (stereoisomer 1) |
| I-87 | (1S,2R)-N-[(9R*)-3,9-dimethyl-10-oxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]-2-(5-methylpyrimidin-2-yl)cyclopropane-1-carboxamide (stereoisomer 2) |
| I-88 | (1S,2R)-N-[3-(difluoromethyl)-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]-2-(5-methylpyridin-2-yl)cyclopropane-1-carboxamide |
| I-89 | (1S,2R)-N-{3-methyl-8,11-dioxa-3,4,16,17- |
| | tetraazatetracyclo[10.7.0.0\hat{ }{2,6}.0\hat{ }{13,17}]nonadeca-1(12),2(6),4,13,15,18-hexaen- |
| | 15-yl}-2-(5-methylpyridin-2-yl)cyclopropane-1-carboxamide |
| I-90 | (1S,2R)-N-{3-methyl-9-oxa-3,4,14,15- |
| | tetraazatetracyclo[8.7.0.0\hat{ }{2,6}.0\hat{ }{11,15}]heptadeca-1(10),2(6),4,11,13,16-hexaen- |
| | 13-yl}-2-(5-methylpyridin-2-yl)cyclopropane-1-carboxamide |
| I-91 | 3-{3-methyl-7,11-dioxa-3,4,16,17- |
| | tetraazatetracyclo[10.7.0.0\hat{ }{2,6}.0\hat{ }{13,17}]nonadeca-1(12),2(6),4,13,15,18-hexaen- |
| | 15-yl}-1-[(5-methylpyridin-2-yl)methyl]urea |
| I-92 | (1S,2R)-2-(5-cyclopropylpyrimidin-2-yl)-N-[(9R)-3,9-dimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]cyclopropane-1-carboxamide |
| I-93 | (1S,2R)-N-[(8R)-8-ethyl-3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]-2-(5-methylpyrimidin-2-yl)cyclopropane-1-carboxamide |
| I-94 | (1S,2R)-N-[(9R)-9-ethyl-3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]-2-(5-methylpyrimidin-2-yl)cyclopropane-1-carboxamide |
| I-95 | rel-1-[(1R)-1-(5-cyclopropylpyridin-2-yl)ethyl]-3-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}urea (stereoisomer 1) |
| I-96 | rel-1-[(1R)-1-(5-cyclopropylpyridin-2-yl)ethyl]-3-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}urea (stereoisomer 2) |
| I-97 | (1S,2R)-2-(4,5-dimethylpyrimidin-2-yl)-N-{3-methyl-10-oxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}cyclopropane-1-carboxamide |
| I-98 | 1-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}-3-[1-(5-methylpyridin-2-yl)cyclopropyl]urea |
| I-99 | 1-{[5-(difluoromethyl)pyridin-2-yl]methyl}-3-{3-methyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl}urea |
| I- | (1S,2R)-2-(5-cyclopropylpyrimidin-2-yl)-N-[(8R)-3,8-dimethyl-7,10-dioxa-3,4,15,16- |
| 100 | tetraazatetracyclo[9.7.0.0\hat{ }{2,6}.0\hat{ }{12,16}]octadeca-1(11),2(6),4,12,14,17-hexaen- |
| | 14-yl]cyclopropane-1-carboxamide |
| I- | (1S,2R)-N-[(9R,13R)-3,10-dimethyl-7,14-dioxa-3,4,10,19,20- |
| 101 | pentaazapentacyclo[13.7.0.0\hat{ }{2,6}.0\hat{ }{9,13}.0\hat{ }{16,20}]docosa- |
| | 1(15),2(6),4,16,18,21-hexaen-18-yl]-2-(5-methylpyridin-2-yl)cyclopropane-1- |
| | carboxamide |
| I- | (1S,2R)-N-[(8R,12R)-3,11-dimethyl-7,14-dioxa-3,4,11,19,20- |
| 102 | pentaazapentacyclo[13.7.0.0\hat{ }{2,6}.0\hat{ }{8,12}.0\hat{ }{16,20}]docosa- |
| | 1(15),2(6),4,16,18,21-hexaen-18-yl]-2-(5-methylpyridin-2-yl)cyclopropane-1- |
| | carboxamide |
| I- | (1S,2R)-N-[(8R)-3,8-dimethyl-7,10-dioxa-3,4,15,16- |
| 103 | tetraazatetracyclo[9.7.0.0\hat{ }{2,6}.0\hat{ }{12,16}]octadeca-1(11),2(6),4,12,14,17-hexaen- |
| | 14-yl]-2-(4,5-dimethylpyrimidin-2-yl)cyclopropane-1-carboxamide |
| I- | (1S,2R)-N-[(9R)-3,9-dimethyl-7,10-dioxa-3,4,15,16- |
| 104 | tetraazatetracyclo[9.7.0.0\hat{ }{2,6}.0\hat{ }{12,16}]octadeca-1(11),2(6),4,12,14,17-hexaen- |
| | 14-yl]-2-[5-(fluoromethyl)pyridin-2-yl]cyclopropane-1-carboxamide |
| I- | (1S,2R)-2-[5-(difluoromethyl)pyrimidin-2-yl]-N-[(9R)-3,9-dimethyl-7,10-dioxa- |
| 105 | 3,4,15,16-tetraazatetracyclo[9.7.0.0\hat{ }{2,6}.0\hat{ }{12,16}]octadeca-1(11),2(6),4,12,14,17- |
| | hexaen-14-yl]cyclopropane-1-carboxamide |
| I- | (1S,2R)-2-(4,5-dimethylpyrimidin-2-yl)-N-{3-methyl-7,11-dioxa-3,4,16,17- |
| 106 | tetraazatetracyclo[10.7.0.0\hat{ }{2,6}.0\hat{ }{13,17}]nonadeca-1(12),2(6),4,13,15,18-hexaen- |
| | 15-yl}cyclopropane-1-carboxamide |
| I- | (1S,2R)-2-[5-(difluoromethyl)pyrimidin-2-yl]-N-{3-methyl-7,11-dioxa-3,4,16,17- |
| 107 | tetraazatetracyclo[10.7.0.0\hat{ }{2,6}.0\hat{ }{13,17}]nonadeca-1(12),2(6),4,13,15,18-hexaen- |
| | 15-yl}cyclopropane-1-carboxamide |
| I- | (1S,2R)-N-[(9R)-3,9-dimethyl-7,10-dioxa-3,4,15,16- |
| 108 | tetraazatetracyclo[9.7.0.0\hat{ }{2,6}.0\hat{ }{12,16}]octadeca-1(11),2(6),4,12,14,17-hexaen- |
| | 14-yl]-2-(4,5-dimethylpyrimidin-2-yl)cyclopropane-1-carboxamide |
| I- | (1S,2R)-2-(5-cyclopropylpyrimidin-2-yl)-N-{3-methyl-7,11-dioxa-3,4,16,17- |
| 109 | tetraazatetracyclo[10.7.0.0\hat{ }{2,6}.0\hat{ }{13,17}]nonadeca-1(12),2(6),4,13,15,18-hexaen- |
| | 15-yl}cyclopropane-1-carboxamide |
| I- | (1S,2R)-2-(5-methoxypyrazin-2-yl)-N-{3-methyl-7,11-dioxa-3,4,16,17- |
| 110 | tetraazatetracyclo[10.7.0.0\hat{ }{2,6}.0\hat{ }{13,17}]nonadeca-1(12),2(6),4,13,15,18-hexaen- |
| | 15-yl}cyclopropane-1-carboxamide |
| I- | (1S,2R)-2-(4,6-dimethylpyrimidin-2-yl)-N-{3-methyl-7,11-dioxa-3,4,16,17- |
| 111 | tetraazatetracyclo[10.7.0.0\hat{ }{2,6}.0\hat{ }{13,17}]nonadeca-1(12),2(6),4,13,15,18-hexaen- |
| | 15-yl}cyclopropane-1-carboxamide |
| I- | (1S,2R)-N-{3-methyl-7,11-dioxa-3,4,16,17- |
| 112 | tetraazatetracyclo[10.7.0.0\hat{ }{2,6}.0\hat{ }{13,17}]nonadeca-1(12),2(6),4,13,15,18-hexaen- |
| | 15-yl}-2-(5-methylpyrazin-2-yl)cyclopropane-1-carboxamide |
| I- | (1S,2R)-2-(5-cyclopropylpyrimidin-2-yl)-N-[(9R*)-3,9-dimethyl-10-oxa-3,4,15,16- |
| 113 | tetraazatetracyclo[9.7.0.0\hat{ }{2,6}.0\hat{ }{12,16}]octadeca-1(11),2(6),4,12,14,17-hexaen- |
| | 14-yl]cyclopropane-1-carboxamide (stereoisomer 1) |
| I- | (1S,2R)-2-(5-cyclopropylpyrimidin-2-yl)-N-[(9R*)-3,9-dimethyl-10-oxa-3,4,15,16- |
| 114 | tetraazatetracyclo[9.7.0.0\hat{ }{2,6}.0\hat{ }{12,16}]octadeca-1(11),2(6),4,12,14,17-hexaen- |
| | 14-yl]cyclopropane-1-carboxamide (stereoisomer 2) |
| I- | (1S,2R)-2-(5-methylpyrimidin-2-yl)-N-[(8R,9R)-3,8,9-trimethyl-7,10-dioxa- |
| 115 | 3,4,15,16-tetraazatetracyclo[9.7.0.0\hat{ }{2,6}.0\hat{ }{12,16}]octadeca-1(11),2(6),4,12,14,17- |
| | hexaen-14-yl]cyclopropane-1-carboxamide |
| I- | (1S,2R)-2-[5-(difluoromethyl)pyrimidin-2-yl]-N-[(8R,9R)-3,8,9-trimethyl-7,10-dioxa- |
| 116 | 3,4,15,16-tetraazatetracyclo[9.7.0.0\hat{ }{2,6}.0\hat{ }{12,16}]octadeca-1(11),2(6),4,12,14,17- |
| | hexaen-14-yl]cyclopropane-1-carboxamide |
| I- | 1-{[5-(fluoromethyl)pyridin-2-yl]methyl}-3-{3-methyl-7,10-dioxa-3,4,15,16- |
| 117 | tetraazatetracyclo[9.7.0.0\hat{ }{2,6}.0\hat{ }{12,16}]octadeca-1(11),2(6),4,12,14,17-hexaen- |
| | 14-yl}urea |
| I- | (1S,2R)-N-[(8R*)-8-hydroxy-3,8-dimethyl-10-oxa-3,4,15,16- |
| 118 | tetraazatetracyclo[9.7.0.0\hat{ }{2,6}.0\hat{ }{12,16}]octadeca-1(11),2(6),4,12,14,17-hexaen- |
| | 14-yl]-2-(5-methylpyridin-2-yl)cyclopropane-1-carboxamide (stereoisomer 1) |
| I- | (1S,2R)-N-[(8R*)-8-hydroxy-3,8-dimethyl-10-oxa-3,4,15,16- |
| 119 | tetraazatetracyclo[9.7.0.0\hat{ }{2,6}.0\hat{ }{12,16}]octadeca-1(11),2(6),4,12,14,17-hexaen- |
| | 14-yl]-2-(5-methylpyridin-2-yl)cyclopropane-1-carboxamide (stereoisomer 2) |
| I- | (1S,2R)-N-[(8R)-3,8-dimethyl-7,10-dioxa-3,4,15,16- |
| 120 | tetraazatetracyclo[9.7.0.0\hat{ }{2,6}.0\hat{ }{12,16}]octadeca-1(11),2(6),4,12,14,17-hexaen- |
| | 14-yl]-2-[5-(fluoromethyl)pyridin-2-yl]cyclopropane-1-carboxamide |
| I- | rel-(1R,2S)-2-(4-methyl-1H-pyrazol-1-yl)-N-{3-methyl-7,10-dioxa-3,4,15,16- |
| 121 | tetraazatetracyclo[9.7.0.0\hat{ }{2,6}.0\hat{ }{12,16}]octadeca-1(11),2(6),4,12,14,17-hexaen- |
| | 14-yl}cyclopropane-1-carboxamide (stereoisomer 1) |
| I- | rel-(1R,2S)-2-(4-methyl-1H-pyrazol-1-yl)-N-{3-methyl-7,10-dioxa-3,4,15,16- |
| 122 | tetraazatetracyclo[9.7.0.0\hat{ }{2,6}.0\hat{ }{12,16}]octadeca-1(11),2(6),4,12,14,17-hexaen- |
| | 14-yl}cyclopropane-1-carboxamide (stereoisomer 2) |
| I- | (1S,2R)-2-(5-cyclopropylpyrimidin-2-yl)-N-[(8R,9R)-3,8,9-trimethyl-7,10-dioxa- |
| 123 | 3,4,15,16-tetraazatetracyclo[9.7.0.0\hat{ }{2,6}.0\hat{ }{12,16}]octadeca-1(11),2(6),4,12,14,17- |
| | hexaen-14-yl]cyclopropane-1-carboxamide |
| I- | (1S,2R)-N-{3-methyl-7,11-dioxa-3,4,16,17- |
| 124 | tetraazatetracyclo[10.7.0.0\hat{ }{2,6}.0\hat{ }{13,17}]nonadeca-1(12),2(6),4,13,15,18-hexaen- |
| | 15-yl}-2-[5-(trifluoromethyl)pyrimidin-2-yl]cyclopropane-1-carboxamide |
| I- | rel-3-{3-methyl-7,11-dioxa-3,4,16,17- |
| 125 | tetraazatetracyclo[10.7.0.0\hat{ }{2,6}.0\hat{ }{13,17}]nonadeca-1(12),2(6),4,13,15,18-hexaen- |
| | 15-yl}-1-[(1R)-1-(5-methylpyrimidin-2-yl)ethyl]urea (stereoisomer 1) |
| I- | rel-3-{3-methyl-7,11-dioxa-3,4,16,17- |
| 126 | tetraazatetracyclo[10.7.0.0\hat{ }{2,6}.0\hat{ }{13,17}]nonadeca-1(12),2(6),4,13,15,18-hexaen- |
| | 15-yl}-1-[(1R)-1-(5-methylpyrimidin-2-yl)ethyl]urea (stereoisomer 2) |
| I- | (1S,2R)-N-[3-(difluoromethyl)-7,11-dioxa-3,4,16,17- |
| 127 | tetraazatetracyclo[10.7.0.0\hat{ }{2,6}.0\hat{ }{13,17}]nonadeca-1(12),2(6),4,13,15,18-hexaen- |
| | 15-yl]-2-(5-methylpyrimidin-2-yl)cyclopropane-1-carboxamide |
| I- | (1S,2R)-N-[(8R*)-8-(hydroxymethyl)-3,8-dimethyl-9-oxa-3,4,14,15- |
| 128 | tetraazatetracyclo[8.7.0.0\hat{ }{2,6}.0\hat{ }{11,15}]heptadeca-1(10),2(6),4,11,13,16-hexaen- |
| | 13-yl]-2-(5-methylpyridin-2-yl)cyclopropane-1-carboxamide (stereoisomer 1) |
| I- | (1S,2R)-N-[(8R*)-8-(hydroxymethyl)-3,8-dimethyl-9-oxa-3,4,14,15- |
| 129 | tetraazatetracyclo[8.7.0.0\hat{ }{2,6}.0\hat{ }{11,15}]heptadeca-1(10),2(6),4,11,13,16-hexaen- |
| | 13-yl]-2-(5-methylpyridin-2-yl)cyclopropane-1-carboxamide (stereoisomer 2) |
| I- | (1S,2R)-N-{3-methyl-10-oxa-4,5,6,15,16- |
| 130 | pentaazatetracyclo[9.7.0.0\hat{ }{2,6}.0\hat{ }{12,16}]octadeca-1(11),2,4,12,14,17-hexaen-14- |
| | yl}-2-(5-methylpyridin-2-yl)cyclopropane-1-carboxamide |
| I- | (1S,2R)-2-[5-(difluoromethyl)pyrimidin-2-yl]-N-[(10R)-3,10-dimethyl-7,11-dioxa- |
| 131 | 3,4,16,17-tetraazatetracyclo[10.7.0.0\hat{ }{2,6}.0\hat{ }{13,17}]nonadeca- |
| | 1(12),2(6),4,13,15,18-hexaen-15-yl]cyclopropane-1-carboxamide |
| I- | (1S,2R)-2-(5-methylpyrimidin-2-yl)-N-[(8R,10R)-3,8,10-trimethyl-7,11-dioxa- |
| 132 | 3,4,16,17-tetraazatetracyclo[10.7.0.0\hat{ }{2,6}.0\hat{ }{13,17}]nonadeca- |
| | 1(12),2(6),4,13,15,18-hexaen-15-yl]cyclopropane-1-carboxamide |
| I- | (1S,2R)-2-[5-(difluoromethyl)pyrimidin-2-yl]-N-[(8R,10R)-3,8,10-trimethyl-7,11- |
| 133 | dioxa-3,4,16,17-tetraazatetracyclo[10.7.0.0\hat{ }{2,6}.0\hat{ }{13,17}]nonadeca- |
| | 1(12),2(6),4,13,15,18-hexaen-15-yl]cyclopropane-1-carboxamide |
| I- | 3-[(9R)-3,9-dimethyl-7,10-dioxa-3,4,15,16- |
| 134 | tetraazatetracyclo[9.7.0.0\hat{ }{2,6}.0\hat{ }{12,16}]octadeca-1(11),2(6),4,12,14,17-hexaen- |
| | 14-yl]-1-[(5-methylpyrimidin-2-yl)methyl]urea |
| I- | N-{3-methyl-7,11-dioxa-3,4,16,17- |
| 135 | tetraazatetracyclo[10.7.0.0\hat{ }{2,6}.0\hat{ }{13,17}]nonadeca-1(12),2(6),4,13,15,18-hexaen- |
| | 15-yl}-3-(5-methylpyridin-2-yl)propanamide |
| I- | (1S,2R)-N-{3-methyl-7,10-dioxa-3,4,12,15- |
| 136 | tetraazatetracyclo[9.7.0.0\hat{ }{2,6}.0\hat{ }{12,16}]octadeca-1(11),2(6),4,13,15,17-hexaen- |
| | 14-yl}-2-(5-methylpyridin-2-yl)cyclopropane-1-carboxamide |
| I- | (1S,2R)-N-{3,10-dimethyl-7-oxa-3,4,10,16,17- |
| 137 | pentaazatetracyclo[10.7.0.0\hat{ }{2,6}.0\hat{ }{13,17}]nonadeca-1(12),2(6),4,13,15,18-hexaen- |
| | 15-yl}-2-(5-methylpyridin-2-yl)cyclopropane-1-carboxamide |
| I- | (1S,2R)-2-[5-(difluoromethyl)pyrimidin-2-yl]-N-[(9R*)-3,9-dimethyl-10-oxa- |
| 138 | 3,4,15,16-tetraazatetracyclo[9.7.0.0\hat{ }{2,6}.0\hat{ }{12,16}]octadeca-1(11),2(6),4,12,14,17- |
| | hexaen-14-yl]cyclopropane-1-carboxamide (stereoisomer 1) |
| I- | (1S,2R)-2-[5-(difluoromethyl)pyrimidin-2-yl]-N-[(9R*)-3,9-dimethyl-10-oxa- |
| 139 | 3,4,15,16-tetraazatetracyclo[9.7.0.0\hat{ }{2,6}.0\hat{ }{12,16}]octadeca-1(11),2(6),4,12,14,17- |
| | hexaen-14-yl]cyclopropane-1-carboxamide (stereoisomer 2) |
| I- | (1S,2R)-2-[5-(difluoromethyl)pyrimidin-2-yl]-N-[(8R)-3,8-dimethyl-7,11-dioxa- |
| 140 | 3,4,16,17-tetraazatetracyclo[10.7.0.0\hat{ }{2,6}.0\hat{ }{13,17}]nonadeca- |
| | 1(12),2(6),4,13,15,18-hexaen-15-yl]cyclopropane-1-carboxamide |
| I- | 3-[(9R)-3,9-dimethyl-7,10-dioxa-3,4,15,16- |
| 141 | tetraazatetracyclo[9.7.0.0\hat{ }{2,6}.0\hat{ }{12,16}]octadeca-1(11),2(6),4,12,14,17-hexaen- |
| | 14-yl]-1-[(1R*)-1-(5-methylpyrimidin-2-yl)ethyl]urea (stereoisomer 1) |
| I- | 3-[(9R)-3,9-dimethyl-7,10-dioxa-3,4,15,16- |
| 142 | tetraazatetracyclo[9.7.0.0\hat{ }{2,6}.0\hat{ }{12,16}]octadeca-1(11),2(6),4,12,14,17-hexaen- |
| | 14-yl]-1-[(1R*)-1-(5-methylpyrimidin-2-yl)ethyl]urea (stereoisomer 2) |
| I- | rel-3-{3-methyl-7,10-dioxa-3,4,15,16- |
| 143 | tetraazatetracyclo[9.7.0.0\hat{ }{2,6}.0\hat{ }{12,16}]octadeca-1(11),2(6),4,12,14,17-hexaen- |
| | 14-yl}-1-[(1R)-1-(5-methylpyrimidin-2-yl)ethyl]urea (stereoisomer 1) |
| I- | rel-3-{3-methyl-7,10-dioxa-3,4,15,16- |
| 144 | tetraazatetracyclo[9.7.0.0\hat{ }{2,6}.0\hat{ }{12,16}]octadeca-1(11),2(6),4,12,14,17-hexaen- |
| | 14-yl}-1-[(1R)-1-(5-methylpyrimidin-2-yl)ethyl]urea (stereoisomer 2) |
| I- | N-[(9R)-3,9-dimethyl-7,10-dioxa-3,4,15,16- |
| 145 | tetraazatetracyclo[9.7.0.0\hat{ }{2,6}.0\hat{ }{12,16}]octadeca-1(11),2(6),4,12,14,17-hexaen- |
| | 14-yl]-3-(5-methylpyrimidin-2-yl)propanamide |
| I- | (1S,2R)-N-{3,8-dimethyl-3,4,8,15,16- |
| 146 | pentaazatetracyclo[9.7.0.0\hat{ }{2,6}.0\hat{ }{12,16}]octadeca-1(11),2(6),4,12,14,17-hexaen- |
| | 14-yl}-2-(5-methylpyridin-2-yl)cyclopropane-1-carboxamide |
| I- | (1S,2R)-N-{3-methyl-9-oxo-7,11-dioxa-3,4,16,17- |
| 147 | tetraazatetracyclo[10.7.0.0\hat{ }{2,6}.0\hat{ }{13,17}]nonadeca-1(12),2(6),4,13,15,18-hexaen- |
| | 15-yl}-2-(5-methylpyridin-2-yl)cyclopropane-1-carboxamide |
| I- | (1S,2R)-N-{4-methyl-11-oxa-5,16,17- |
| 148 | triazatetracyclo[10.7.0.0\hat{ }{2,7}.0\hat{ }{13,17}]nonadeca-1(12),2(7),3,5,13,15,18-heptaen- |
| | 15-yl}-2-(5-methylpyrimidin-2-yl)cyclopropane-1-carboxamide |
| I- | (1S,2R)-2-[5-(difluoromethyl)pyridin-2-yl]-N-[(9R*)-9-hydroxy-3,9-dimethyl-7,11- |
| 149 | dioxa-3,4,16,17-tetraazatetracyclo[10.7.0.0\hat{ }{2,6}.0\hat{ }{13,17}]nonadeca- |
| | 1(12),2(6),4,13,15,18-hexaen-15-yl]cyclopropane-1-carboxamide (stereoisomer 1) |
| I- | (1S,2R)-2-[5-(difluoromethyl)pyridin-2-yl]-N-[(9R*)-9-hydroxy-3,9-dimethyl-7,11- |
| 150 | dioxa-3,4,16,17-tetraazatetracyclo[10.7.0.0\hat{ }{2,6}.0\hat{ }{13,17}]nonadeca- |
| | 1(12),2(6),4,13,15,18-hexaen-15-yl]cyclopropane-1-carboxamide (stereoisomer 2) |
| I- | (1S,2R)-2-(5-chloropyrimidin-2-yl)-N-{3-methyl-7,11-dioxa-3,4,16,17- |
| 151 | tetraazatetracyclo[10.7.0.0\hat{ }{2,6}.0\hat{ }{13,17}]nonadeca-1(12),2(6),4,13,15,18-hexaen- |
| | 15-yl}cyclopropane-1-carboxamide |
| I- | (1S,2R)-N-{3-methyl-5,10-dioxa-4,15,16- |
| 152 | triazatetracyclo[9.7.0.0\hat{ }{2,6}.0\hat{ }{12,16}]octadeca-1(11),2(6),3,12,14,17-hexaen-14- |
| | yl}-2-(5-methylpyrimidin-2-yl)cyclopropane-1-carboxamide |
| I- | (1S,2R)-2-[5-(difluoromethyl)pyrimidin-2-yl]-N-{3-methyl-10-oxa-3,4,15,16- |
| 153 | tetraazatetracyclo[9.7.0.0\hat{ }{2,6}.0\hat{ }{12,16}]octadeca-1(11),2(6),4,12,14,17-hexaen- |
| | 14-yl}cyclopropane-1-carboxamide |
| I- | (1S,2R)-N-[(9R)-3,9-dimethyl-7,10-dioxa-3,4,15,16- |
| 154 | tetraazatetracyclo[9.7.0.0\hat{ }{2,6}.0\hat{ }{12,16}]octadeca-1(11),2(6),4,12,14,17-hexaen- |
| | 14-yl]-2-[5-(3-fluoroazetidin-1-yl)pyridin-2-yl]cyclopropane-1-carboxamide |
| I- | (1S,2R)-2-[5-(3,3-difluoroazetidin-1-yl)pyridin-2-yl]-N-[(9R)-3,9-dimethyl-7,10- |
| 155 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]cyclopropane-1-carboxamide |
| I- | (1S,2R)-2-(5-{bicyclo[1.1.1]pentan-1-yl}pyrimidin-2-yl)-N-[(9R)-3,9-dimethyl-7,10- |
| 156 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]cyclopropane-1-carboxamide |
| I- | (1S,2R)-N-{3-methyl-7,12-dioxa-3,4,17,18- |
| 157 | tetraazatetracyclo[11.7.0.0\hat{ }{2,6}.0\hat{ }{14,18}]icosa-1(13),2(6),4,14,16,19-hexaen-16- |
| | yl}-2-(5-methylpyrimidin-2-yl)cyclopropane-1-carboxamide |
| I- | (1S,2R)-N-[(9R)-3,9-dimethyl-7,10-dioxa-3,4,15,16- |
| 158 | tetraazatetracyclo[9.7.0.0\hat{ }{2,6}.0\hat{ }{12,16}]octadeca-1(11),2(6),4,12,14,17-hexaen- |
| | 14-yl]-2-{5-[(2R)-2-methylazetidin-1-yl]pyrimidin-2-yl}cyclopropane-1-carboxamide |
| I- | (1S,2R)-2-[5-(1,1-difluoroethyl)pyrimidin-2-yl]-N-[(9R)-3,9-dimethyl-7,10-dioxa- |
| 159 | 3,4,15,16-tetraazatetracyclo[9.7.0.0\hat{ }{2,6}.0\hat{ }{12,16}]octadeca-1(11),2(6),4,12,14,17- |
| | hexaen-14-yl]cyclopropane-1-carboxamide |
| I- | (1S,2R)-N-[(9R*)-9-(difluoromethyl)-3-methyl-7,10-dioxa-3,4,15,16- |
| 160 | tetraazatetracyclo[9.7.0.0\hat{ }{2,6}.0\hat{ }{12,16}]octadeca-1(11),2(6),4,12,14,17-hexaen- |
| | 14-yl]-2-(5-methylpyrimidin-2-yl)cyclopropane-1-carboxamide (stereoisomer 1) |
| I- | (1S,2R)-N-[(9R*)-9-(difluoromethyl)-3-methyl-7,10-dioxa-3,4,15,16- |
| 161 | tetraazatetracyclo[9.7.0.0\hat{ }{2,6}.0\hat{ }{12,16}]octadeca-1(11),2(6),4,12,14,17-hexaen- |
| | 14-yl]-2-(5-methylpyrimidin-2-yl)cyclopropane-1-carboxamide (stereoisomer 2) |
| I- | (3R*)-N-[(9R)-3,9-dimethyl-7,10-dioxa-3,4,15,16- |
| 162 | tetraazatetracyclo[9.7.0.0\hat{ }{2,6}.0\hat{ }{12,16}]octadeca-1(11),2(6),4,12,14,17-hexaen- |
| | 14-yl]-4,4-difluoro-3-(4-methyl-1H-pyrazol-1-yl)butanamide (stereoisomer 1) |
| I- | (1S,2R)-N-[(9R)-3,9-dimethyl-7,10-dioxa-3,4,15,16- |
| 163 | tetraazatetracyclo[9.7.0.0\hat{ }{2,6}.0\hat{ }{12,16}]octadeca-1(11),2(6),4,12,14,17-hexaen- |
| | 14-yl]-2-[5-(3-methylazetidin-1-yl)pyridin-2-yl]cyclopropane-1-carboxamide |
| I- | (3R*)-N-[(9R)-3,9-dimethyl-7,10-dioxa-3,4,15,16- |
| 164 | tetraazatetracyclo[9.7.0.0\hat{ }{2,6}.0\hat{ }{12,16}]octadeca-1(11),2(6),4,12,14,17-hexaen- |
| | 14-yl]-4,4-difluoro-3-(4-methyl-1H-pyrazol-1-yl)butanamide (stereoisomer 2) |
| I- | (1S,2R)-2-[5-(azetidin-1-yl)pyrimidin-2-yl]-N-[(9R)-3,9-dimethyl-7,10-dioxa- |
| 165 | 3,4,15,16-tetraazatetracyclo[9.7.0.0\hat{ }{2,6}.0\hat{ }{12,16}]octadeca-1(11),2(6),4,12,14,17- |
| | hexaen-14-yl]cyclopropane-1-carboxamide |
| I- | (1S,2R)-N-{3-methyl-7,11-dioxa-3,4,16,17- |
| 166 | tetraazatetracyclo[10.7.0.0\hat{ }{2,6}.0\hat{ }{13,17}]nonadeca-1(12),2(6),4,13,15,18-hexaen- |
| | 15-yl}-2-(5-{2-oxabicyclo[2.1.1]hexan-4-yl}pyrimidin-2-yl)cyclopropane-1- |
| | carboxamide |
| I- | (1S,2R)-N-[(9R)-3,9-dimethyl-7,10-dioxa-3,4,15,16- |
| 167 | tetraazatetracyclo[9.7.0.0\hat{ }{2,6}.0\hat{ }{12,16}]octadeca-1(11),2(6),4,12,14,17-hexaen- |
| | 14-yl]-2-(5-{2-oxabicyclo[2.1.1]hexan-4-yl}pyrimidin-2-yl)cyclopropane-1- |
| | carboxamide |
| I- | (1S,2R)-2-(5-cyanopyrimidin-2-yl)-N-[(9R)-3,9-dimethyl-7,10-dioxa-3,4,15,16- |
| 168 | tetraazatetracyclo[9.7.0.0\hat{ }{2,6}.0\hat{ }{12,16}]octadeca-1(11),2(6),4,12,14,17-hexaen- |
| | 14-yl]cyclopropane-1-carboxamide |
| I- | (3R*)-N-[(9R)-3,9-dimethyl-7,10-dioxa-3,4,15,16- |
| 169 | tetraazatetracyclo[9.7.0.0\hat{ }{2,6}.0\hat{ }{12,16}]octadeca-1(11),2(6),4,12,14,17-hexaen- |
| | 14-yl]-3-(5-methylpyridin-2-yl)butanamide (stereoisomer 1) |
| I- | (3R*)-N-[(9R)-3,9-dimethyl-7,10-dioxa-3,4,15,16- |
| 170 | tetraazatetracyclo[9.7.0.0\hat{ }{2,6}.0\hat{ }{12,16}]octadeca-1(11),2(6),4,12,14,17-hexaen- |
| | 14-yl]-3-(5-methylpyridin-2-yl)butanamide (stereoisomer 2) |
| I- | (1S,2R)-2-[5-(3,3-difluoroazetidin-1-yl)pyrimidin-2-yl]-N-[(9R)-3,9-dimethyl-7,10- |
| 171 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]cyclopropane-1-carboxamide |
| I- | (1S,2R)-2-(5-methylpyrimidin-2-yl)-N-[(8RS,9SR)-3,8,9-trimethyl-7,10-dioxa- |
| 172 | 3,4,15,16-tetraazatetracyclo[9.7.0.0\hat{ }{2,6}.0\hat{ }{12,16}]octadeca-1(11),2(6),4,12,14,17- |
| | hexaen-14-yl]cyclopropane-1-carboxamide |
| I- | (1S,2R)-N-[(9R)-3,9-dimethyl-7,10-dioxa-3,4,15,16- |
| 173 | tetraazatetracyclo[9.7.0.0\hat{ }{2,6}.0\hat{ }{12,16}]octadeca-1(11),2(6),4,12,14,17-hexaen- |
| | 14-yl]-2-[5-(3-fluoroazetidin-1-yl)pyrimidin-2-yl]cyclopropane-1-carboxamide |
| I- | (1S,2R)-N-[(9R)-3,9-dimethyl-7,10-dioxa-3,4,15,16- |
| 174 | tetraazatetracyclo[9.7.0.0\hat{ }{2,6}.0\hat{ }{12,16}]octadeca-1(11),2(6),4,12,14,17-hexaen- |
| | 14-yl]-2-[5-(3-methylazetidin-1-yl)pyrimidin-2-yl]cyclopropane-1-carboxamide |
| I- | (1S,2R)-N-[(9R*)-9-(dimethylamino)-3-methyl-7,11-dioxa-3,4,16,17- |
| 175 | tetraazatetracyclo[10.7.0.0\hat{ }{2,6}.0\hat{ }{13,17}]nonadeca-1(12),2(6),4,13,15,18-hexaen- |
| | 15-yl]-2-(5-methylpyridin-2-yl)cyclopropane-1-carboxamide (stereoisomer 1) |
| I- | (1S,2R)-N-[(9R*)-9-(dimethylamino)-3-methyl-7,11-dioxa-3,4,16,17- |
| 176 | tetraazatetracyclo[10.7.0.0\hat{ }{2,6}.0\hat{ }{13,17}]nonadeca-1(12),2(6),4,13,15,18-hexaen- |
| | 15-yl]-2-(5-methylpyridin-2-yl)cyclopropane-1-carboxamide (stereoisomer 2) |
| I- | (1S,2R)-N-[(9R)-3,9-dimethyl-7,10-dioxa-3,4,15,16- |
| 177 | tetraazatetracyclo[9.7.0.0\hat{ }{2,6}.0\hat{ }{12,16}]octadeca-1(11),2(6),4,12,14,17-hexaen- |
| | 14-yl]-2-[5-(1-hydroxycyclopropyl)pyrimidin-2-yl]cyclopropane-1-carboxamide |
| I- | (1S,2R)-N-[(9R)-3,9-dimethyl-7,10-dioxa-3,4,15,16- |
| 178 | tetraazatetracyclo[9.7.0.0\hat{ }{2,6}.0\hat{ }{12,16}]octadeca-1(11),2(6),4,12,14,17-hexaen- |
| | 14-yl]-2-{5-[1-(dimethylamino)cyclopropyl]pyridin-2-yl}cyclopropane-1- |
| | carboxamide |
| I- | N-[(9R)-3,9-dimethyl-7,10-dioxa-3,4,15,16- |
| 179 | tetraazatetracyclo[9.7.0.0\hat{ }{2,6}.0\hat{ }{12,16}]octadeca-1(11),2(6),4,12,14,17-hexaen- |
| | 14-yl]-2-(1-methyl-1H-pyrazol-3-yl)acetamide |
| I- | (1S,2R)-N-[(8R*)-3,8-dimethyl-9-oxa-3,4,14,15- |
| 180 | tetraazatetracyclo[8.7.0.0\hat{ }{2,6}.0\hat{ }{11,15}]heptadeca-1(10),2(6),4,11,13,16-hexaen- |
| | 13-yl]-2-(5-methylpyrimidin-2-yl)cyclopropane-1-carboxamide (stereoisomer 2) |
| I- | (1S,2R)-2-(5-cyclopropylpyrimidin-2-yl)-N-[(8R*)-3,8-dimethyl-9-oxa-3,4,14,15- |
| 181 | tetraazatetracyclo[8.7.0.0\hat{ }{2,6}.0\hat{ }{11,15}]heptadeca-1(10),2(6),4,11,13,16-hexaen- |
| | 13-yl]cyclopropane-1-carboxamide (stereoisomer 1) |
| I- | (1S,2R)-2-(5-cyclopropylpyrimidin-2-yl)-N-[(8R*)-3,8-dimethyl-9-oxa-3,4,14,15- |
| 182 | tetraazatetracyclo[8.7.0.0\hat{ }{2,6}.0\hat{ }{11,15}]heptadeca-1(10),2(6),4,11,13,16-hexaen- |
| | 13-yl]cyclopropane-1-carboxamide (stereoisomer 2) |
| I- | (1S,2R)-N-[(8R*)-3,8-dimethyl-9-oxa-3,4,14,15- |
| 183 | tetraazatetracyclo[8.7.0.0\hat{ }{2,6}.0\hat{ }{11,15}]heptadeca-1(10),2(6),4,11,13,16-hexaen- |
| | 13-yl]-2-(5-methylpyrimidin-2-yl)cyclopropane-1-carboxamide (stereoisomer 1) |
| I- | (1S,2R)-2-[5-(difluoromethyl)pyrimidin-2-yl]-N-[(8R*)-3,8-dimethyl-9-oxa- |
| 184 | 3,4,14,15-tetraazatetracyclo[8.7.0.0\hat{ }{2,6}.0\hat{ }{11,15}]heptadeca- |
| | 1(10),2(6),4,11,13,16-hexaen-13-yl]cyclopropane-1-carboxamide (stereoisomer 1) |
| I- | (1S,2R)-2-[5-(difluoromethyl)pyrimidin-2-yl]-N-[(8R*)-3,8-dimethyl-9-oxa- |
| 185 | 3,4,14,15-tetraazatetracyclo[8.7.0.0\hat{ }{2,6}.0\hat{ }{11,15}]heptadeca- |
| | 1(10),2(6),4,11,13,16-hexaen-13-yl]cyclopropane-1-carboxamide (stereoisomer 2) |
| I- | (1S,2R)-2-{5-[1-(difluoromethyl)cyclopropyl]pyrimidin-2-yl}-N-[(9R)-3,9-dimethyl- |
| 186 | 7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]cyclopropane-1-carboxamide |
| I- | (1S,2R)-2-{5-[(1R*)-2,2-difluorocyclopropyl]pyrimidin-2-yl}-N-[(9R)-3,9-dimethyl- |
| 187 | 7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]cyclopropane-1-carboxamide (stereoisomer 1) |
| I- | (1S,2R)-2-{5-[(1R*)-2,2-difluorocyclopropyl]pyrimidin-2-yl}-N-[(9R)-3,9-dimethyl- |
| 188 | 7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]cyclopropane-1-carboxamide (stereoisomer 2) |
| I- | (1S,2R)-N-[(9R)-3,9-dimethyl-7,10-dioxa-3,4,15,16- |
| 189 | tetraazatetracyclo[9.7.0.0\hat{ }{2,6}.0\hat{ }{12,16}]octadeca-1(11),2(6),4,12,14,17-hexaen- |
| | 14-yl]-2-[5-(1-fluorocyclopropyl)pyrimidin-2-yl]cyclopropane-1-carboxamide |
| I- | (1S,2R)-2-[5-(2H3)methylpyrimidin-2-yl]-N-[(8R,9R)-3,8,9-trimethyl-7,10-dioxa- |
| 190 | 3,4,15,16-tetraazatetracyclo[9.7.0.0\hat{ }{2,6}.0\hat{ }{12,16}]octadeca-1(11),2(6),4,12,14,17- |
| | hexaen-14-yl]cyclopropane-1-carboxamide |
| I- | (1S,2R)-N-[(9R)-3,9-dimethyl-10-oxa-3,4,15,16- |
| 191 | tetraazatetracyclo[9.7.0.0\hat{ }{2,6}.0\hat{ }{12,16}]octadeca-1(11),2(6),4,12,14,17-hexaen- |
| | 14-yl]-2-[5-(2H3)methylpyrimidin-2-yl]cyclopropane-1-carboxamide |
| I- | (1R*)-1-(5-methylpyrimidin-2-yl)ethylN-[(9R)-3,9-dimethyl-7,10-dioxa-3,4,15,16- |
| 192 | tetraazatetracyclo[9.7.0.0\hat{ }{2,6}.0\hat{ }{12,16}]octadeca-1(11),2(6),4,12,14,17-hexaen- |
| | 14-yl]carbamate (stereoisomer 1) |
| I- | (1R*)-1-(5-methylpyrimidin-2-yl)ethylN-[(9R)-3,9-dimethyl-7,10-dioxa-3,4,15,16- |
| 193 | tetraazatetracyclo[9.7.0.0\hat{ }{2,6}.0\hat{ }{12,16}]octadeca-1(11),2(6),4,12,14,17-hexaen- |
| | 14-yl]carbamate (stereoisomer 2) |
| I- | 1-[(1R)-1-[5-(difluoromethyl)pyrimidin-2-yl]ethyl]-3-[(9R)-3,9-dimethyl-7,10-dioxa- |
| 194 | 3,4,15,16-tetraazatetracyclo[9.7.0.0\hat{ }{2,6}.0\hat{ }{12,16}]octadeca-1(11),2(6),4,12,14,17- |
| | hexaen-14-yl]urea |
| I- | (1S,2R)-N-[(9R)-3,9-dimethyl-7,10-dioxa-3,4,15,16- |
| 195 | tetraazatetracyclo[9.7.0.0\hat{ }{2,6}.0\hat{ }{12,16}]octadeca-1(11),2(6),4,12,14,17-hexaen- |
| | 14-yl]-2-[5-(3-methyloxetan-3-yl)pyridin-2-yl]cyclopropane-1-carboxamide |
| I- | (1S,2R)-2-[5-(3,3-difluoroazetidin-1-yl)pyrimidin-2-yl]-N-[(8R,9R)-3,8,9-trimethyl- |
| 196 | 7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]cyclopropane-1-carboxamide |
| I- | 1-[(1R)-1-[5-(difluoromethyl)pyridin-2-yl]ethyl]-3-{3-methyl-7,10-dioxa-3,4,15,16- |
| 197 | tetraazatetracyclo[9.7.0.0\hat{ }{2,6}.0\hat{ }{12,16}]octadeca-1(11),2(6),4,12,14,17-hexaen- |
| | 14-yl}urea |
| I- | (1S,2R)-2-(5-{3-azabicyclo[3.1.0]hexan-3-yl}pyrimidin-2-yl)-N-[(9R)-3,9-dimethyl- |
| 198 | 7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]cyclopropane-1-carboxamide |
| I- | (1S,2R)-2-[5-(3-fluoroazetidin-1-yl)pyrimidin-2-yl]-N-[(8R,9R)-3,8,9-trimethyl-7,10- |
| 199 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]cyclopropane-1-carboxamide |
| I- | 3-[(9R)-3,9-dimethyl-7,10-dioxa-3,4,15,16- |
| 200 | tetraazatetracyclo[9.7.0.0\hat{ }{2,6}.0\hat{ }{12,16}]octadeca-1(11),2(6),4,12,14,17-hexaen- |
| | 14-yl]-1-[(8R)-5,6,7,8-tetrahydroquinolin-8-yl]urea |
| I- | 3-[(9R)-3,9-dimethyl-7,10-dioxa-3,4,15,16- |
| 201 | tetraazatetracyclo[9.7.0.0\hat{ }{2,6}.0\hat{ }{12,16}]octadeca-1(11),2(6),4,12,14,17-hexaen- |
| | 14-yl]-1-[(8S)-5,6,7,8-tetrahydroquinolin-8-yl]urea |
| I- | 1-{[5-(difluoromethyl)pyridin-2-yl]methyl}-3-[(8R,9R)-3,8,9-trimethyl-7,10-dioxa- |
| 202 | 3,4,15,16-tetraazatetracyclo[9.7.0.0\hat{ }{2,6}.0\hat{ }{12,16}]octadeca-1(11),2(6),4,12,14,17- |
| | hexaen-14-yl]urea |
| I- | (1S,2R)-N-[(9R)-3,9-dimethyl-7,10-dioxa-3,4,15,16- |
| 203 | tetraazatetracyclo[9.7.0.0\hat{ }{2,6}.0\hat{ }{12,16}]octadeca-1(11),2(6),4,12,14,17-hexaen- |
| | 14-yl]-2-{5-[(2R*)-2-methyloxetan-2-yl]pyridin-2-yl}cyclopropane-1-carboxamide |
| | (stereoisomer 1) |
| I- | (1S,2R)-N-[(9R)-3,9-dimethyl-7,10-dioxa-3,4,15,16- |
| 204 | tetraazatetracyclo[9.7.0.0\hat{ }{2,6}.0\hat{ }{12,16}]octadeca-1(11),2(6),4,12,14,17-hexaen- |
| | 14-yl]-2-{5-[(2R*)-2-methyloxetan-2-yl]pyridin-2-yl}cyclopropane-1-carboxamide |
| | (stereoisomer 2) |
| I- | (1S,2R)-2-[5-(2-hydroxypropan-2-yl)pyridin-2-yl]-N-[(8R,9R)-3,8,9-trimethyl-7,10- |
| 205 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]cyclopropane-1-carboxamide |
| I- | (1S,2R)-2-[5-(2-hydroxypropan-2-yl)pyrazin-2-yl]-N-[(8R,9R)-3,8,9-trimethyl-7,10- |
| 206 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]cyclopropane-1-carboxamide |
| I- | (1S,2R)-2-[5-(3,3-difluoroazetidin-1-yl)pyrazin-2-yl]-N-[(9R)-3,9-dimethyl-7,10- |
| 207 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]cyclopropane-1-carboxamide |
| I- | (1S,2R)-2-[5-(difluoromethyl)pyridin-2-yl]-N-[(9R)-3-methyl-7,16-dioxa- |
| 208 | 3,4,13,21,22-pentaazapentacyclo[15.7.0.0\hat{ }{2,6}.0\hat{ }{9,13}.0\hat{ }{18,22}]tetracosa- |
| | 1(17),2(6),4,18,20,23-hexaen-20-yl]cyclopropane-1-carboxamide |
| I- | (1S,2R)-N-[(9R)-3,9-dimethyl-7,10-dioxa-3,4,15,16- |
| 209 | tetraazatetracyclo[9.7.0.0\hat{ }{2,6}.0\hat{ }{12,16}]octadeca-1(11),2(6),4,12,14,17-hexaen- |
| | 14-yl]-2-(5-{2-oxabicyclo[2.1.1]hexan-4-yl}pyridin-2-yl)cyclopropane-1- |
| | carboxamide |
| I- | (1S,2R)-2-[5-(3,3-difluoroazetidin-1-yl)pyrazin-2-yl]-N-[(8R,9R)-3,8,9-trimethyl- |
| 210 | 7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]cyclopropane-1-carboxamide |
| I- | 3-[(9R)-3,9-dimethyl-7,10-dioxa-3,4,15,16- |
| 211 | tetraazatetracyclo[9.7.0.0\hat{ }{2,6}.0\hat{ }{12,16}]octadeca-1(11),2(6),4,12,14,17-hexaen- |
| | 14-yl]-1-[(1R*)-2,2,2-trifluoro-1-(5-methylpyrimidin-2-yl)ethyl]urea (stereoisomer 1) |
| I- | 3-[(9R)-3,9-dimethyl-7,10-dioxa-3,4,15,16- |
| 212 | tetraazatetracyclo[9.7.0.0\hat{ }{2,6}.0\hat{ }{12,16}]octadeca-1(11),2(6),4,12,14,17-hexaen- |
| | 14-yl]-1-[(1R*)-2,2,2-trifluoro-1-(5-methylpyrimidin-2-yl)ethyl]urea (stereoisomer 2) |
| I- | (1S,2R)-2-[5-(difluoromethyl)pyrimidin-2-yl]-N-{3-methyl-7,10-dioxa-13-thia- |
| 213 | 3,4,15-triazatetracyclo[9.7.0.0\hat{ }{2,6}.0\hat{ }{12,16}]octadeca-1(11),2(6),4,12(16),14,17- |
| | hexaen-14-yl}cyclopropane-1-carboxamide |
| I- | (1S,2R)-2-(5-{2-oxabicyclo[2.1.1]hexan-4-yl}pyrimidin-2-yl)-N-[(8R,9R)-3,8,9- |
| 214 | trimethyl-7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]cyclopropane-1-carboxamide |
| I- | rel-3-{3-methyl-7,10-dioxa-13-thia-3,4,15- |
| 215 | triazatetracyclo[9.7.0.0\hat{ }{2,6}.0\hat{ }{12,16}]octadeca-1(18),2(6),4,11,14,16-hexaen-14- |
| | yl}-1-[(1R)-1-(5-methylpyrimidin-2-yl)ethyl]urea (stereoisomer 1) |
| I- | rel-3-{3-methyl-7,10-dioxa-13-thia-3,4,15- |
| 216 | triazatetracyclo[9.7.0.0\hat{ }{2,6}.0\hat{ }{12,16}]octadeca-1(18),2(6),4,11,14,16-hexaen-14- |
| | yl}-1-[(1R)-1-(5-methylpyrimidin-2-yl)ethyl]urea (stereoisomer 2) |
| I- | (1S,2R)-N-[(14S)-3-methyl-7,16-dioxa-3,4,10,21,22- |
| 217 | pentaazapentacyclo[15.7.0.0\hat{ }{2,6}.0\hat{ }{10,14}.0\hat{ }{18,22}]tetracosa- |
| | 1(17),2(6),4,18,20,23-hexaen-20-yl]-2-(5-methylpyrimidin-2-yl)cyclopropane-1- |
| | carboxamide |
| I- | (1S,2R)-2-[5-(difluoromethyl)pyridin-2-yl]-N-[(14S)-3-methyl-7,16-dioxa- |
| 218 | 3,4,10,21,22-pentaazapentacyclo[15.7.0.0\hat{ }{2,6}.0\hat{ }{10,14}.0\hat{ }{18,22}]tetracosa- |
| | 1(17),2(6),4,18,20,23-hexaen-20-yl]cyclopropane-1-carboxamide |
| I- | (1S,2R)-N-[(9R)-3,9-dimethyl-7,10-dioxa-3,4,15,16- |
| 219 | tetraazatetracyclo[9.7.0.0\hat{ }{2,6}.0\hat{ }{12,16}]octadeca-1(11),2(6),4,12,14,17-hexaen- |
| | 14-yl]-2-(5-{3-fluorobicyclo[1.1.1]pentan-1-yl}pyrimidin-2-yl)cyclopropane-1- |
| | carboxamide |
| I- | (1S,2R)-2-[5-(difluoromethyl)pyrimidin-2-yl]-N-[(8R,12R)-3-methyl-7,14-dioxa- |
| 220 | 3,4,11,19,20-pentaazapentacyclo[13.7.0.0\hat{ }{2,6}.0\hat{ }{8,12}.0\hat{ }{16,20}]docosa- |
| | 1(15),2(6),4,16,18,21-hexaen-18-yl]cyclopropane-1-carboxamide |
| I- | (1S,2R)-2-[5-(difluoromethyl)pyridin-2-yl]-N-[(14S)-3-methyl-9-oxo-7,16-dioxa- |
| 221 | 3,4,10,21,22-pentaazapentacyclo[15.7.0.0\hat{ }{2,6}.0\hat{ }{10,14}.0\hat{ }{18,22}]tetracosa- |
| | 1(17),2(6),4,18,20,23-hexaen-20-yl]cyclopropane-1-carboxamide |
| I- | 1-[(1R)-1-[5-(difluoromethyl)pyrimidin-2-yl]ethyl]-3-[(8R,9R)-3,8,9-trimethyl-7,10- |
| 222 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]urea |
| I- | (1S,2R)-2-[5-(difluoromethyl)pyrimidin-2-yl]-N-[(8RS,11SR)-3-methyl-7,12-dioxa- |
| 223 | 3,4,17,18-tetraazapentacyclo[11.7.0.0\hat{ }{2,6}.0\hat{ }{8,11}.0\hat{ }{14,18}]icosa- |
| | 1(13),2(6),4,14,16,19-hexaen-16-yl]cyclopropane-1-carboxamide |
| I- | 3-{9,9-difluoro-3-methyl-7,11-dioxa-3,4,16,17- |
| 224 | tetraazatetracyclo[10.7.0.0\hat{ }{2,6}.0\hat{ }{13,17}]nonadeca-1(12),2(6),4,13,15,18-hexaen- |
| | 15-yl}-1-[(1R)-1-[5-(difluoromethyl)pyrimidin-2-yl]ethyl]urea |
| I- | (1S,2R)-2-(5-{1-methyl-2-oxabicyclo[2.1.1]hexan-4-yl}pyrimidin-2-yl)-N-[(8R,9R)- |
| 225 | 3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]cyclopropane-1-carboxamide |
| I- | (1S,2R)-2-(5-methylpyrimidin-2-yl)-N-[(8R*,9R*)-3,8,9-trimethyl-7,10-dioxa- |
| 226 | 3,4,12,15-tetraazatetracyclo[9.7.0.0\hat{ }{2,6}.0\hat{ }{12,16}]octadeca-1(11),2(6),4,13,15,17- |
| | hexaen-14-yl]cyclopropane-1-carboxamide (stereoisomer 1) |
| I- | (1S,2R)-2-(5-methylpyrimidin-2-yl)-N-[(8R*,9R*)-3,8,9-trimethyl-7,10-dioxa- |
| 227 | 3,4,12,15-tetraazatetracyclo[9.7.0.0\hat{ }{2,6}.0\hat{ }{12,16}]octadeca-1(11),2(6),4,13,15,17- |
| | hexaen-14-yl]cyclopropane-1-carboxamide (stereoisomer 2) |
| I- | (1S,2R)-2-[5-(difluoromethyl)pyridin-2-yl]-N-[(8R,12R)-3-methyl-7,14-dioxa- |
| 228 | 3,4,11,19,20-pentaazapentacyclo[13.7.0.0\hat{ }{2,6}.0\hat{ }{8,12}.0\hat{ }{16,20}]docosa- |
| | 1(15),2(6),4,16,18,21-hexaen-18-yl]cyclopropane-1-carboxamide |
| I- | (1S,2R)-2-[5-(difluoromethyl)pyridin-2-yl]-N-[(8R,12R)-3,11-dimethyl-7,14-dioxa- |
| 229 | 3,4,11,19,20-pentaazapentacyclo[13.7.0.0\hat{ }{2,6}.0\hat{ }{8,12}.0\hat{ }{16,20}]docosa- |
| | 1(15),2(6),4,16,18,21-hexaen-18-yl]cyclopropane-1-carboxamide |
| I- | 2-(1-methyl-1H-pyrazol-3-yl)-N-[(8R,9R)-3,8,9-trimethyl-7,10-dioxa-3,4,15,16- |
| 230 | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | (1S,2R)-2-[5-(difluoromethyl)pyridin-2-yl]-N-[(9R)-3-methyl-14-oxo-7,16-dioxa- |
| 231 | 3,4,13,21,22-pentaazapentacyclo[15.7.0.0\hat{ }{2,6}.0\hat{ }{9,13}.0\hat{ }{18,22}]tetracosa- |
| | 1(17),2(6),4,18,20,23-hexaen-20-yl]cyclopropane-1-carboxamide |
| I- | (1S,2R)-2-[5-(difluoromethyl)pyridin-2-yl]-N-[3-(1-hydroxyethyl)-5,10-dioxa- |
| 232 | 4,15,16-triazatetracyclo[9.7.0.0\hat{ }{2,6}.0\hat{ }{12,16}]octadeca-1(11),2(6),3,12,14,17- |
| | hexaen-14-yl]cyclopropane-1-carboxamide |
| I- | (1S,2R)-N-[(9R)-3-methyl-7,16-dioxa-3,4,13,21,22- |
| 233 | pentaazapentacyclo[15.7.0.0\hat{ }{2,6}.0\hat{ }{9,13}.0\hat{ }{18,22}]tetracosa- |
| | 1(17),2(6),4,18,20,23-hexaen-20-yl]-2-(5-methylpyrimidin-2-yl)cyclopropane-1- |
| | carboxamide |
| I- | 2-(1-methyl-1H-pyrazol-3-yl)-N-[(8R,12R)-3-methyl-7,14-dioxa-3,4,11,19,20- |
| 234 | pentaazapentacyclo[13.7.0.0\hat{ }{2,6}.0\hat{ }{8,12}.0\hat{ }{16,20}]docosa- |
| | 1(15),2(6),4,16,18,21-hexaen-18-yl]acetamide |
| I- | (3R*)-3-(5-methylpyrimidin-2-yl)-N-[(8R,9R)-3,8,9-trimethyl-7,10-dioxa-3,4,15,16- |
| 235 | tetraazatetracyclo[9.7.0.0\hat{ }{2,6}.0\hat{ }{12,16}]octadeca-1(11),2(6),4,12,14,17-hexaen- |
| | 14-yl]butanamide (stereoisomer 1) |
| I- | 1-{[(2S)-oxolan-2-yl]methyl}-3-[(8R,9R)-3,8,9-trimethyl-7,10-dioxa-3,4,15,16- |
| 236 | tetraazatetracyclo[9.7.0.0\hat{ }{2,6}.0\hat{ }{12,16}]octadeca-1(11),2(6),4,12,14,17-hexaen- |
| | 14-yl]urea |
| I- | (1S,2R)-2-[5-(difluoromethyl)pyridin-2-yl]-N-[8-(hydroxymethyl)-3,8-dimethyl-9- |
| 237 | oxa-3,4,14,15-tetraazatetracyclo[8.7.0.0\hat{ }{2,6}.0\hat{ }{11,15}]heptadeca- |
| | 1(10),2(6),4,11,13,16-hexaen-13-yl]cyclopropane-1-carboxamide |
| I- | (3R*)-3-(5-methylpyrimidin-2-yl)-N-[(8R,9R)-3,8,9-trimethyl-7,10-dioxa-3,4,15,16- |
| 238 | tetraazatetracyclo[9.7.0.0\hat{ }{2,6}.0\hat{ }{12,16}]octadeca-1(11),2(6),4,12,14,17-hexaen- |
| | 14-yl]butanamide (stereoisomer 2) |
| I- | 2-(2-methyl-1,3-thiazol-4-yl)-N-[(8R,9R)-3,8,9-trimethyl-7,10-dioxa-3,4,15,16- |
| 239 | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 3-(1-methyl-1H-pyrazol-3-yl)-N-[(8R,9R)-3,8,9-trimethyl-7,10-dioxa-3,4,15,16- |
| 240 | tetraazatetracyclo[9.7.0.0\hat{ }{2,6}.0\hat{ }{12,16}]octadeca-1(11),2(6),4,12,14,17-hexaen- |
| | 14-yl]propanamide |
| I- | N-[(8R,12R)-3,11-dimethyl-7,14-dioxa-3,4,11,19,20- |
| 241 | pentaazapentacyclo[13.7.0.0\hat{ }{2,6}.0\hat{ }{8,12}.0\hat{ }{16,20}]docosa- |
| | 1(15),2(6),4,16,18,21-hexaen-18-yl]-2-(1-methyl-1H-pyrazol-3-yl)acetamide |
| I- | 3-[(1R,2S)-2-methoxycyclobutyl]-1-[(8R,9R)-3,8,9-trimethyl-7,10-dioxa-3,4,15,16- |
| 242 | tetraazatetracyclo[9.7.0.0\hat{ }{2,6}.0\hat{ }{12,16}]octadeca-1(11),2(6),4,12,14,17-hexaen- |
| | 14-yl]urea |
| I- | (1S,2R)-2-[5-(difluoromethyl)pyrimidin-2-yl]-N-[(8R,12R)-3,11-dimethyl-7,14- |
| 243 | dioxa-3,4,11,19,20-pentaazapentacyclo[13.7.0.0\hat{ }{2,6}.0\hat{ }{8,12}.0\hat{ }{16,20}]docosa- |
| | 1(15),2(6),4,16,18,21-hexaen-18-yl]cyclopropane-1-carboxamide |
| I- | 3-[4-(3,3,3-trifluoropropanoyl)piperazin-1-yl]-N-[(8R,9R)-3,8,9-trimethyl-7,10-dioxa- |
| 244 | 3,4,15,16-tetraazatetracyclo[9.7.0.0\hat{ }{2,6}.0\hat{ }{12,16}]octadeca-1(11),2(6),4,12,14,17- |
| | hexaen-14-yl]propanamide |
| I- | (3R*)-4,4-difluoro-3-(5-methylpyrimidin-2-yl)-N-[(8R,9R)-3,8,9-trimethyl-7,10- |
| 245 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]butanamide (stereoisomer 1) |
| I- | (3R*)-4,4-difluoro-3-(5-methylpyrimidin-2-yl)-N-[(8R,9R)-3,8,9-trimethyl-7,10- |
| 246 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]butanamide (stereoisomer 2) |
| I- | 2-{5H,6H,7H-pyrazolo[3,2-b][1,3]oxazin-2-yl}-N-[(8R,9R)-3,8,9-trimethyl-7,10- |
| 247 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide |
| I- | (1S,2R)-2-(5-{2-oxabicyclo[2.1.1]hexan-1-yl}pyrimidin-2-yl)-N-[(8R,9R)-3,8,9- |
| 248 | trimethyl-7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]cyclopropane-1-carboxamide |
| I- | 2-{pyrazolo[1,5-a]pyridin-2-yl}-N-[(8R,9R)-3,8,9-trimethyl-7,10-dioxa-3,4,15,16- |
| 249 | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | (1S,2R)-2-[5-(difluoromethyl)pyridin-2-yl]-N-[(2R*)-3′-methyl-7′,11′-dioxa- |
| 250 | 3′,4′,16′,17′-tetraazaspiro[oxetane-2,9′- |
| | tetracyclo[10.7.0.0\hat{ }{2,6}.0\hat{ }{13,17}]nonadecane]-1′(12′),2′(6′),4′,13′,15′,18′-hexaen- |
| | 15′-yl]cyclopropane-1-carboxamide (stereoisomer 1) |
| I- | 1-[(1R)-1-[5-(difluoromethyl)pyrimidin-2-yl]ethyl]-3-{3-methyl-7,11-dioxa- |
| 251 | 3,4,16,17-tetraazatetracyclo[10.7.0.0\hat{ }{2,6}.0\hat{ }{13,17}]nonadeca- |
| | 1(12),2(6),4,13,15,18-hexaen-15-yl}urea |
| I- | (1S,2R)-2-[5-(difluoromethyl)pyridin-2-yl]-N-[(2R*)-3′-methyl-7′,11′-dioxa- |
| 252 | 3′,4′,16′,17′-tetraazaspiro[oxetane-2,9′- |
| | tetracyclo[10.7.0.0\hat{ }{2,6}.0\hat{ }{13,17}]nonadecane]-1′(12′),2′(6′),4′,13′,15′,18′-hexaen- |
| | 15′-yl]cyclopropane-1-carboxamide (stereoisomer 2) |
| I- | 1-[(1R)-1-[5-(difluoromethyl)pyrimidin-2-yl]ethyl]-3-{3-methyl-7,10-dioxa- |
| 253 | 3,4,15,16-tetraazatetracyclo[9.7.0.0\hat{ }{2,6}.0\hat{ }{12,16}]octadeca-1(11),2(6),4,12,14,17- |
| | hexaen-14-yl}urea |
| I- | 2-[(8R*)-5,6,7,8-tetrahydroquinolin-8-yl]-N-[(8R,9R)-3,8,9-trimethyl-7,10-dioxa- |
| 254 | 3,4,15,16-tetraazatetracyclo[9.7.0.0\hat{ }{2,6}.0\hat{ }{12,16}]octadeca-1(11),2(6),4,12,14,17- |
| | hexaen-14-yl]acetamide (stereoisomer 1) |
| I- | 2-[(8R*)-5,6,7,8-tetrahydroquinolin-8-yl]-N-[(8R,9R)-3,8,9-trimethyl-7,10-dioxa- |
| 255 | 3,4,15,16-tetraazatetracyclo[9.7.0.0\hat{ }{2,6}.0\hat{ }{12,16}]octadeca-1(11),2(6),4,12,14,17- |
| | hexaen-14-yl]acetamide (stereoisomer 2) |
| I- | 3-[(2R*)-oxolan-2-yl]-N-[(8R,9R)-3,8,9-trimethyl-7,10-dioxa-3,4,15,16- |
| 256 | tetraazatetracyclo[9.7.0.0\hat{ }{2,6}.0\hat{ }{12,16}]octadeca-1(11),2(6),4,12,14,17-hexaen- |
| | 14-yl]propanamide (stereoisomer 1) |
| I- | 3-[(2R*)-oxolan-2-yl]-N-[(8R,9R)-3,8,9-trimethyl-7,10-dioxa-3,4,15,16- |
| 257 | tetraazatetracyclo[9.7.0.0\hat{ }{2,6}.0\hat{ }{12,16}]octadeca-1(11),2(6),4,12,14,17-hexaen- |
| | 14-yl]propanamide (stereoisomer 2) |
| I- | (1S,2R)-N-[(8R*)-8-(2-hydroxypropan-2-yl)-3-methyl-9-oxa-3,4,14,15- |
| 258 | tetraazatetracyclo[8.7.0.0\hat{ }{2,6}.0\hat{ }{11,15}]heptadeca-1(10),2(6),4,11,13,16-hexaen- |
| | 13-yl]-2-(5-methylpyridin-2-yl)cyclopropane-1-carboxamide (stereoisomer 1) |
| I- | (1S,2R)-N-[(8R*)-8-(2-hydroxypropan-2-yl)-3-methyl-9-oxa-3,4,14,15- |
| 259 | tetraazatetracyclo[8.7.0.0\hat{ }{2,6}.0\hat{ }{11,15}]heptadeca-1(10),2(6),4,11,13,16-hexaen- |
| | 13-yl]-2-(5-methylpyridin-2-yl)cyclopropane-1-carboxamide (stereoisomer 2) |
| I- | (1S,2R)-N-{3-methyl-7,10-dioxa-3,4,15,16- |
| 260 | tetraazatetracyclo[9.7.0.0\hat{ }{2,6}.0\hat{ }{12,16}]octadeca-1(11),2(6),4,12,14,17-hexaen- |
| | 14-yl}-2-[5-(propan-2-yloxy)pyridin-2-yl]cyclopropane-1-carboxamide |
| I- | (1S,2R)-2-[5-(propan-2-yloxy)pyrimidin-2-yl]-N-[(8R,9R)-3,8,9-trimethyl-7,10- |
| 261 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]cyclopropane-1-carboxamide |
| I- | (3R*)-4,4-difluoro-3-(4-methyl-1H-pyrazol-1-yl)-N-[(8R,9R)-3,8,9-trimethyl-7,10- |
| 262 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]butanamide (stereoisomer 1) |
| I- | (3R*)-4,4-difluoro-3-(4-methyl-1H-pyrazol-1-yl)-N-[(8R,9R)-3,8,9-trimethyl-7,10- |
| 263 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]butanamide (stereoisomer 2) |
| I- | 2-(1,5-dimethyl-1H-pyrazol-3-yl)-N-[(8R,9R)-3,8,9-trimethyl-7,10-dioxa-3,4,15,16- |
| 264 | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 3-{[(6R*)-5-oxa-2-azaspiro[3.4]octan-6-yl]methyl}-1-[(8R,9R)-3,8,9-trimethyl-7,10- |
| 265 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]urea (stereoisomer 1) |
| I- | 3-{[(6R*)-5-oxa-2-azaspiro[3.4]octan-6-yl]methyl}-1-[(8R,9R)-3,8,9-trimethyl-7,10- |
| 266 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]urea (stereoisomer 2) |
| I- | (1R*)-1-(5-methylpyrimidin-2-yl)ethylN-[(8R,9R)-3,8,9-trimethyl-7,10-dioxa- |
| 267 | 3,4,15,16-tetraazatetracyclo[9.7.0.0\hat{ }{2,6}.0\hat{ }{12,16}]octadeca-1(11),2(6),4,12,14,17- |
| | hexaen-14-yl]carbamate (stereoisomer 1) |
| I- | (1R*)-1-(5-methylpyrimidin-2-yl)ethylN-[(8R,9R)-3,8,9-trimethyl-7,10-dioxa- |
| 268 | 3,4,15,16-tetraazatetracyclo[9.7.0.0\hat{ }{2,6}.0\hat{ }{12,16}]octadeca-1(11),2(6),4,12,14,17- |
| | hexaen-14-yl]carbamate (stereoisomer 2) |
| I- | (1S,2R)-2-[5-(difluoromethyl)pyridin-2-yl]-N-[(8R*)-8-(hydroxymethyl)-3,8- |
| 269 | dimethyl-9-oxa-3,4,14,15-tetraazatetracyclo[8.7.0.0\hat{ }{2,6}.0\hat{ }{11,15}]heptadeca- |
| | 1(10),2(6),4,11,13,16-hexaen-13-yl]cyclopropane-1-carboxamide (stereoisomer 1) |
| I- | (1S,2R)-2-[5-(difluoromethyl)pyridin-2-yl]-N-[(8R*)-8-(hydroxymethyl)-3,8- |
| 270 | dimethyl-9-oxa-3,4,14,15-tetraazatetracyclo[8.7.0.0\hat{ }{2,6}.0\hat{ }{11,15}]heptadeca- |
| | 1(10),2(6),4,11,13,16-hexaen-13-yl]cyclopropane-1-carboxamide (stereoisomer 2) |
| I- | (6aR*,7S*,7aS*)-N-[(8R,9R)-3,8,9-trimethyl-7,10-dioxa-3,4,15,16- |
| 271 | tetraazatetracyclo[9.7.0.0\hat{ }{2,6}.0\hat{ }{12,16}]octadeca-1(11),2(6),4,12,14,17-hexaen- |
| | 14-yl]-5H,6H,6aH,7H,7aH-cyclopropa[h]quinoline-7-carboxamide (stereoisomer 1) |
| I- | (6aR*,7S*,7aS*)-N-[(8R,9R)-3,8,9-trimethyl-7,10-dioxa-3,4,15,16- |
| 272 | tetraazatetracyclo[9.7.0.0\hat{ }{2,6}.0\hat{ }{12,16}]octadeca-1(11),2(6),4,12,14,17-hexaen- |
| | 14-yl]-5H,6H,6aH,7H,7aH-cyclopropa[h]quinoline-7-carboxamide (stereoisomer 2) |
| I- | 2-{1-[2-(dimethylamino)ethyl]-1H-pyrazol-3-yl}-N-[(8R,9R)-3,8,9-trimethyl-7,10- |
| 273 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide (stereoisomer 1) |
| I- | 2-{1-[2-(dimethylamino)ethyl]-1H-pyrazol-5-yl}-N-[(8R,9R)-3,8,9-trimethyl-7,10- |
| 274 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide (stereoisomer 2) |
| I- | 2-(1-{bicyclo[1.1.1]pentan-1-yl}-1H-pyrazol-3-yl)-N-[(9R)-3,9-dimethyl-7,10-dioxa- |
| 275 | 3,4,15,16-tetraazatetracyclo[9.7.0.0\hat{ }{2,6}.0\hat{ }{12,16}]octadeca-1(11),2(6),4,12,14,17- |
| | hexaen-14-yl]acetamide |
| I- | 1-[5-(difluoromethyl)pyrimidin-2-yl]ethylN-[(9R)-3,9-dimethyl-7,10-dioxa- |
| 276 | 3,4,15,16-tetraazatetracyclo[9.7.0.0\hat{ }{2,6}.0\hat{ }{12,16}]octadeca-1(11),2(6),4,12,14,17- |
| | hexaen-14-yl]carbamate |
| I- | (2R*)-N-[(9R)-3,9-dimethyl-7,10-dioxa-3,4,15,16- |
| 277 | tetraazatetracyclo[9.7.0.0\hat{ }{2,6}.0\hat{ }{12,16}]octadeca-1(11),2(6),4,12,14,17-hexaen- |
| | 14-yl]-2-(1-methyl-1H-pyrazol-3-yl)propanamide (stereoisomer 1) |
| I- | (2R*)-N-[(9R)-3,9-dimethyl-7,10-dioxa-3,4,15,16- |
| 278 | tetraazatetracyclo[9.7.0.0\hat{ }{2,6}.0\hat{ }{12,16}]octadeca-1(11),2(6),4,12,14,17-hexaen- |
| | 14-yl]-2-(1-methyl-1H-pyrazol-3-yl)propanamide (stereoisomer 2) |
| I- | 3-(4-methyl-1H-pyrazol-1-yl)-N-[(8R,9R)-3,8,9-trimethyl-7,10-dioxa-3,4,15,16- |
| 279 | tetraazatetracyclo[9.7.0.0\hat{ }{2,6}.0\hat{ }{12,16}]octadeca-1(11),2(6),4,12,14,17-hexaen- |
| | 14-yl]propanamide |
| I- | 2-(1-cyclopropyl-1H-pyrazol-3-yl)-N-[(9R)-3,9-dimethyl-7,10-dioxa-3,4,15,16- |
| 280 | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | N-[(9R)-3,9-dimethyl-7,10-dioxa-3,4,15,16- |
| 281 | tetraazatetracyclo[9.7.0.0\hat{ }{2,6}.0\hat{ }{12,16}]octadeca-1(11),2(6),4,12,14,17-hexaen- |
| | 14-yl]-2-(5-methylpyrimidin-2-yl)acetamide |
| I- | N-[(9R)-3,9-dimethyl-7,10-dioxa-3,4,15,16- |
| 282 | tetraazatetracyclo[9.7.0.0\hat{ }{2,6}.0\hat{ }{12,16}]octadeca-1(11),2(6),4,12,14,17-hexaen- |
| | 14-yl]-2-(5-methylpyridin-2-yl)acetamide |
| I- | 3-[(1R*)-1-[(2R**)-oxolan-2-yl]ethyl]-1-[(8R,9R)-3,8,9-trimethyl-7,10-dioxa- |
| 283 | 3,4,15,16-tetraazatetracyclo[9.7.0.0\hat{ }{2,6}.0\hat{ }{12,16}]octadeca-1(11),2(6),4,12,14,17- |
| | hexaen-14-yl]urea (stereoisomer 1) |
| I- | 3-[(1R*)-1-[(2R**)-oxolan-2-yl]ethyl]-1-[(8R,9R)-3,8,9-trimethyl-7,10-dioxa- |
| 284 | 3,4,15,16-tetraazatetracyclo[9.7.0.0\hat{ }{2,6}.0\hat{ }{12,16}]octadeca-1(11),2(6),4,12,14,17- |
| | hexaen-14-yl]urea (stereoisomer 2) |
| I- | 3-[(1R*)-1-[(2R**)-oxolan-2-yl]ethyl]-1-[(8R,9R)-3,8,9-trimethyl-7,10-dioxa- |
| 285 | 3,4,15,16-tetraazatetracyclo[9.7.0.0\hat{ }{2,6}.0\hat{ }{12,16}]octadeca-1(11),2(6),4,12,14,17- |
| | hexaen-14-yl]urea (stereoisomer 4) |
| I- | 3-{[(6R*)-5-oxaspiro[3.4]octan-6-yl]methyl}-1-[(8R,9R)-3,8,9-trimethyl-7,10-dioxa- |
| 286 | 3,4,15,16-tetraazatetracyclo[9.7.0.0\hat{ }{2,6}.0\hat{ }{12,16}]octadeca-1(11),2(6),4,12,14,17- |
| | hexaen-14-yl]urea (stereoisomer 1) |
| I- | 3-{[(6R*)-5-oxaspiro[3.4]octan-6-yl]methyl}-1-[(8R,9R)-3,8,9-trimethyl-7,10-dioxa- |
| 287 | 3,4,15,16-tetraazatetracyclo[9.7.0.0\hat{ }{2,6}.0\hat{ }{12,16}]octadeca-1(11),2(6),4,12,14,17- |
| | hexaen-14-yl]urea (stereoisomer 2) |
| I- | 3-{3-methyl-7,10-dioxa-3,4,15,16- |
| 288 | tetraazatetracyclo[9.7.0.0\hat{ }{2,6}.0\hat{ }{12,16}]octadeca-1(11),2(6),4,12,14,17-hexaen- |
| | 14-yl}-1-[(1R)-1-[5-(propan-2-yloxy)pyrimidin-2-yl]ethyl]urea |
| I- | (1S,2R)-2-[5-(difluoromethyl)-3-fluoropyridin-2-yl]-N-[(9R)-3,9-dimethyl-7,10- |
| 289 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]cyclopropane-1-carboxamide |
| I- | rel-1-[(R)-cyclopropyl[5-(difluoromethyl)pyrimidin-2-yl]methyl]-3-{3-methyl-7,10- |
| 290 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl}urea (stereoisomer 1) |
| I- | rel-1-[(R)-cyclopropyl[5-(difluoromethyl)pyrimidin-2-yl]methyl]-3-{3-methyl-7,10- |
| 291 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl}urea (stereoisomer 2) |
| I- | 1-{[(2R*)-1,8-dioxaspiro[4.5]decan-2-yl]methyl}-3-[(8R,9R)-3,8,9-trimethyl-7,10- |
| 292 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]urea (stereoisomer 1) |
| I- | N-[(9R)-3,9-dimethyl-7,10-dioxa-3,4,15,16- |
| 293 | tetraazatetracyclo[9.7.0.0\hat{ }{2,6}.0\hat{ }{12,16}]octadeca-1(11),2(6),4,12,14,17-hexaen- |
| | 14-yl]-2-(2-methyl-1,3-oxazol-4-yl)acetamide |
| I- | 1-{[(2R*)-1,8-dioxaspiro[4.5]decan-2-yl]methyl}-3-[(8R,9R)-3,8,9-trimethyl-7,10- |
| 294 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]urea (stereoisomer 2) |
| I- | (1R*)-1-[5-(difluoromethyl)pyrimidin-2-yl]ethylN-[(9R)-3,9-dimethyl-7,10-dioxa- |
| 295 | 3,4,15,16-tetraazatetracyclo[9.7.0.0\hat{ }{2,6}.0\hat{ }{12,16}]octadeca-1(11),2(6),4,12,14,17- |
| | hexaen-14-yl]carbamate (stereoisomer 2) |
| I- | (1R*)-1-[5-(difluoromethyl)pyrimidin-2-yl]ethylN-[(9R)-3,9-dimethyl-7,10-dioxa- |
| 296 | 3,4,15,16-tetraazatetracyclo[9.7.0.0\hat{ }{2,6}.0\hat{ }{12,16}]octadeca-1(11),2(6),4,12,14,17- |
| | hexaen-14-yl]carbamate (stereoisomer 1) |
| I- | N-[(9R)-3,9-dimethyl-7,10-dioxa-3,4,15,16- |
| 297 | tetraazatetracyclo[9.7.0.0\hat{ }{2,6}.0\hat{ }{12,16}]octadeca-1(11),2(6),4,12,14,17-hexaen- |
| | 14-yl]-2-(6-methylpyridazin-3-yl)acetamide |
| I- | N-[(9R)-3,9-dimethyl-7,10-dioxa-3,4,15,16- |
| 298 | tetraazatetracyclo[9.7.0.0\hat{ }{2,6}.0\hat{ }{12,16}]octadeca-1(11),2(6),4,12,14,17-hexaen- |
| | 14-yl]-2-(2-methyl-1,3-thiazol-4-yl)acetamide |
| I- | 3-[(1R*,4R**)-5-oxaspiro[3.4]octan-1-yl]-1-[(8R,9R)-3,8,9-trimethyl-7,10-dioxa- |
| 299 | 3,4,15,16-tetraazatetracyclo[9.7.0.0\hat{ }{2,6}.0\hat{ }{12,16}]octadeca-1(11),2(6),4,12,14,17- |
| | hexaen-14-yl]urea (stereoisomer 1) |
| I- | 3-[(1R*,4R**)-5-oxaspiro[3.4]octan-1-yl]-1-[(8R,9R)-3,8,9-trimethyl-7,10-dioxa- |
| 300 | 3,4,15,16-tetraazatetracyclo[9.7.0.0\hat{ }{2,6}.0\hat{ }{12,16}]octadeca-1(11),2(6),4,12,14,17- |
| | hexaen-14-yl]urea (stereoisomer 2) |
| I- | 3-[(1R*,4R**)-5-oxaspiro[3.4]octan-1-yl]-1-[(8R,9R)-3,8,9-trimethyl-7,10-dioxa- |
| 301 | 3,4,15,16-tetraazatetracyclo[9.7.0.0\hat{ }{2,6}.0\hat{ }{12,16}]octadeca-1(11),2(6),4,12,14,17- |
| | hexaen-14-yl]urea (stereoisomer 3) |
| I- | 3-[(1R*,4R**)-5-oxaspiro[3.4]octan-1-yl]-1-[(8R,9R)-3,8,9-trimethyl-7,10-dioxa- |
| 302 | 3,4,15,16-tetraazatetracyclo[9.7.0.0\hat{ }{2,6}.0\hat{ }{12,16}]octadeca-1(11),2(6),4,12,14,17- |
| | hexaen-14-yl]urea (stereoisomer 4) |
| I- | (3R*)-N-[(9R)-3,9-dimethyl-7,10-dioxa-3,4,15,16- |
| 303 | tetraazatetracyclo[9.7.0.0\hat{ }{2,6}.0\hat{ }{12,16}]octadeca-1(11),2(6),4,12,14,17-hexaen- |
| | 14-yl]-4,4-difluoro-3-{2H,4H,6H,7H-pyrano[4,3-c]pyrazol-2-yl}butanamide |
| | (stereoisomer 2) |
| I- | (3R*)-N-[(9R)-3,9-dimethyl-7,10-dioxa-3,4,15,16- |
| 304 | tetraazatetracyclo[9.7.0.0\hat{ }{2,6}.0\hat{ }{12,16}]octadeca-1(11),2(6),4,12,14,17-hexaen- |
| | 14-yl]-4,4-difluoro-3-{2H,4H,6H,7H-pyrano[4,3-c]pyrazol-2-yl}butanamide |
| | (stereoisomer 1) |
| I- | N-[(9R)-3,9-dimethyl-7,10-dioxa-3,4,15,16- |
| 305 | tetraazatetracyclo[9.7.0.0\hat{ }{2,6}.0\hat{ }{12,16}]octadeca-1(11),2(6),4,12,14,17-hexaen- |
| | 14-yl]-2-(4-methyl-1,3-thiazol-2-yl)acetamide |
| I- | N-[(9R)-3,9-dimethyl-7,10-dioxa-3,4,15,16- |
| 306 | tetraazatetracyclo[9.7.0.0\hat{ }{2,6}.0\hat{ }{12,16}]octadeca-1(11),2(6),4,12,14,17-hexaen- |
| | 14-yl]-2-(5-methyl-1,3-oxazol-2-yl)acetamide |
| I- | 1-[(3-fluoro-5-methylpyridin-2-yl)methyl]-3-{3-methyl-7,10-dioxa-3,4,15,16- |
| 307 | tetraazatetracyclo[9.7.0.0\hat{ }{2,6}.0\hat{ }{12,16}]octadeca-1(11),2(6),4,12,14,17-hexaen- |
| | 14-yl}urea |
| I- | 1-[(5-{3-fluorobicyclo[1.1.1]pentan-1-yl}pyrimidin-2-yl)methyl]-3-{3-methyl-7,10- |
| 308 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl}urea |
| I- | 1-{[5-(difluoromethyl)-3-fluoropyridin-2-yl]methyl}-3-{3-methyl-7,10-dioxa- |
| 309 | 3,4,15,16-tetraazatetracyclo[9.7.0.0\hat{ }{2,6}.0\hat{ }{12,16}]octadeca-1(11),2(6),4,12,14,17- |
| | hexaen-14-yl}urea |
| I- | (9R)-3,9-dimethyl-N-{[(1S,2R)-2-(5-methylpyridin-2-yl)cyclopropyl]methyl}-7,10- |
| 310 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-amine |
| I- | N-[(9R)-3,9-dimethyl-7,10-dioxa-3,4,15,16- |
| 311 | tetraazatetracyclo[9.7.0.0\hat{ }{2,6}.0\hat{ }{12,16}]octadeca-1(11),2(6),4,12,14,17-hexaen- |
| | 14-yl]-2-(4-methyl-1,3-oxazol-2-yl)acetamide |
| I- | (1S,2R)-2-[5-(difluoromethyl)pyridin-2-yl]-N-{3-[(1R*)-1-hydroxyethyl]-5,10-dioxa- |
| 312 | 4,15,16-triazatetracyclo[9.7.0.0\hat{ }{2,6}.0\hat{ }{12,16}]octadeca-1(11),2(6),3,12,14,17- |
| | hexaen-14-yl}cyclopropane-1-carboxamide (stereoisomer 1) |
| I- | (1S,2R)-2-[5-(difluoromethyl)pyridin-2-yl]-N-{3-[(1R*)-1-hydroxyethyl]-5,10-dioxa- |
| 313 | 4,15,16-triazatetracyclo[9.7.0.0\hat{ }{2,6}.0\hat{ }{12,16}]octadeca-1(11),2(6),3,12,14,17- |
| | hexaen-14-yl}cyclopropane-1-carboxamide (stereoisomer 2) |
| I- | (9R)-3,9-dimethyl-N-[2-(5-methylpyridin-2-yl)ethyl]-7,10-dioxa-3,4,15,16- |
| 314 | tetraazatetracyclo[9.7.0.0\hat{ }{2,6}.0\hat{ }{12,16}]octadeca-1(11),2(6),4,12,14,17-hexaen- |
| | 14-amine |
| I- | N-[(9R)-3,9-dimethyl-7,10-dioxa-3,4,15,16- |
| 315 | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-{4H,6H,7H-pyrazolo[3,2-c][1,4]oxazin-2-yl}-N-[(8R,9R)-3,8,9-trimethyl-7,10- |
| 316 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide |
| I- | 2-(5-methylpyrimidin-2-yl)-N-[(8R,9R)-3,8,9-trimethyl-7,10-dioxa-3,4,15,16- |
| 317 | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-(5-methylpyridin-2-yl)-N-[(8R,9R)-3,8,9-trimethyl-7,10-dioxa-3,4,15,16- |
| 318 | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | (4R*)-N-[(9R)-3,9-dimethyl-7,10-dioxa-3,4,15,16- |
| 319 | tetraazatetracyclo[9.7.0.0\hat{ }{2,6}.0\hat{ }{12,16}]octadeca-1(11),2(6),4,12,14,17-hexaen- |
| | 14-yl]-2-methyl-4,5,6,7-tetrahydro-1,3-benzothiazole-4-carboxamide (stereoisomer 1) |
| I- | (4R*)-N-[(9R)-3,9-dimethyl-7,10-dioxa-3,4,15,16- |
| 320 | tetraazatetracyclo[9.7.0.0\hat{ }{2,6}.0\hat{ }{12,16}]octadeca-1(11),2(6),4,12,14,17-hexaen- |
| | 14-yl]-2-methyl-4,5,6,7-tetrahydro-1,3-benzothiazole-4-carboxamide (stereoisomer 2) |
| I- | 2-[1-(cyclopropylmethyl)-1H-pyrazol-3-yl]-N-[(9R)-3,9-dimethyl-7,10-dioxa- |
| 321 | 3,4,15,16-tetraazatetracyclo[9.7.0.0\hat{ }{2,6}.0\hat{ }{12,16}]octadeca-1(11),2(6),4,12,14,17- |
| | hexaen-14-yl]acetamide |
| I- | 1-[(1R*)-1-[(2R**)-oxolan-2-yl]ethyl]-3-[(8R,9R)-3,8,9-trimethyl-7,10-dioxa- |
| 322 | 3,4,15,16-tetraazatetracyclo[9.7.0.0\hat{ }{2,6}.0\hat{ }{12,16}]octadeca-1(11),2(6),4,12,14,17- |
| | hexaen-14-yl]urea (stereoisomer 3) |
| I- | 2-[1-(cyclopropylmethyl)-1H-pyrazol-5-yl]-N-[(9R)-3,9-dimethyl-7,10-dioxa- |
| 323 | 3,4,15,16-tetraazatetracyclo[9.7.0.0\hat{ }{2,6}.0\hat{ }{12,16}]octadeca-1(11),2(6),4,12,14,17- |
| | hexaen-14-yl]acetamide |
| I- | N-[(9R)-3,9-dimethyl-7,10-dioxa-3,4,15,16- |
| 324 | tetraazatetracyclo[9.7.0.0\hat{ }{2,6}.0\hat{ }{12,16}]octadeca-1(11),2(6),4,12,14,17-hexaen- |
| | 14-yl]-2-{5H,6H,8H-imidazo[2,1-c][1,4]oxazin-2-yl}acetamide |
| I- | (1S,2R)-2-[5-(difluoromethyl)pyrimidin-2-yl]-N-[(8R*,11S*)-3-methyl-7,12-dioxa- |
| 325 | 3,4,17,18-tetraazapentacyclo[11.7.0.0\hat{ }{2,6}.0\hat{ }{8,11}.0\hat{ }{14,18}]icosa- |
| | 1(13),2(6),4,14,16,19-hexaen-16-yl]cyclopropane-1-carboxamide (stereoisomer 1) |
| I- | (1S,2R)-2-[5-(difluoromethyl)pyrimidin-2-yl]-N-[(8R*,11S*)-3-methyl-7,12-dioxa- |
| 326 | 3,4,17,18-tetraazapentacyclo[11.7.0.0\hat{ }{2,6}.0\hat{ }{8,11}.0\hat{ }{14,18}]icosa- |
| | 1(13),2(6),4,14,16,19-hexaen-16-yl]cyclopropane-1-carboxamide (stereoisomer 2) |
| I- | 2-[5-(difluoromethyl)pyridin-2-yl]-N-[(8R,9R)-3,8,9-trimethyl-7,10-dioxa-3,4,15,16- |
| 327 | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | rel-1-[(R)-cyclopropyl(5-methylpyrimidin-2-yl)methyl]-3-{3-methyl-7,10-dioxa- |
| 328 | 3,4,15,16-tetraazatetracyclo[9.7.0.0\hat{ }{2,6}.0\hat{ }{12,16}]octadeca-1(11),2(6),4,12,14,17- |
| | hexaen-14-yl}urea (stereoisomer 1) |
| I- | rel-1-[(R)-cyclopropyl(5-methylpyrimidin-2-yl)methyl]-3-{3-methyl-7,10-dioxa- |
| 329 | 3,4,15,16-tetraazatetracyclo[9.7.0.0\hat{ }{2,6}.0\hat{ }{12,16}]octadeca-1(11),2(6),4,12,14,17- |
| | hexaen-14-yl}urea (stereoisomer 2) |
| I- | (1S,2R)-2-[5-(difluoromethoxy)pyrimidin-2-yl]-N-[(9R)-3,9-dimethyl-7,10-dioxa- |
| 330 | 3,4,15,16-tetraazatetracyclo[9.7.0.0\hat{ }{2,6}.0\hat{ }{12,16}]octadeca-1(11),2(6),4,12,14,17- |
| | hexaen-14-yl]cyclopropane-1-carboxamide |
| I- | N-[(9R)-3,9-dimethyl-7,10-dioxa-3,4,15,16- |
| 331 | tetraazatetracyclo[9.7.0.0\hat{ }{2,6}.0\hat{ }{12,16}]octadeca-1(11),2(6),4,12,14,17-hexaen- |
| | 14-yl]-2-{5-[(1-methyl-1H-pyrazol-4-yl)oxy]pyridin-2-yl}acetamide |
| I- | 3-{3-methyl-7,10-dioxa-3,4,15,16- |
| 332 | tetraazatetracyclo[9.7.0.0\hat{ }{2,6}.0\hat{ }{12,16}]octadeca-1(11),2(6),4,12,14,17-hexaen- |
| | 14-yl}-1-{[5-(pentafluoro-lambda6-sulfanyl)pyridin-2-yl]methyl}urea |
| I- | (1S,2R)-2-(5-{3-fluorobicyclo[1.1.1]pentan-1-yl}pyrimidin-2-yl)-N-{3-methyl-7,10- |
| 333 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl}cyclopropane-1-carboxamide |
| I- | (1S,2R)-N-[(8R*,11S*)-3-methyl-7,12-dioxa-3,4,17,18- |
| 334 | tetraazapentacyclo[11.7.0.0\hat{ }{2,6}.0\hat{ }{8,11}.0\hat{ }{14,18}]icosa-1(13),2(6),4,14,16,19- |
| | hexaen-16-yl]-2-(5-methylpyrimidin-2-yl)cyclopropane-1-carboxamide (stereoisomer 1) |
| I- | (1S,2R)-N-[(8R*,11S*)-3-methyl-7,12-dioxa-3,4,17,18- |
| 335 | tetraazapentacyclo[11.7.0.0\hat{ }{2,6}.0\hat{ }{8,11}.0\hat{ }{14,18}]icosa-1(13),2(6),4,14,16,19- |
| | hexaen-16-yl]-2-(5-methylpyrimidin-2-yl)cyclopropane-1-carboxamide (stereoisomer 2) |
| I- | (1S,2R)-2-[5-(difluoromethyl)pyridin-2-yl]-N-{3-methyl-7,10-dioxa-3,4,12,15- |
| 336 | tetraazatetracyclo[9.7.0.0\hat{ }{2,6}.0\hat{ }{12,16}]octadeca-1(11),2(6),4,13,15,17-hexaen- |
| | 14-yl}cyclopropane-1-carboxamide |
| I- | (1S,2R)-2-[5-(difluoromethyl)pyrimidin-2-yl]-N-[(9R)-3,9-dimethyl-7,10-dioxa- |
| 337 | 3,4,12,15-tetraazatetracyclo[9.7.0.0\hat{ }{2,6}.0\hat{ }{12,16}]octadeca-1(11),2(6),4,13,15,17- |
| | hexaen-14-yl]cyclopropane-1-carboxamide |
| I- | (1S,2R)-2-[5-(difluoromethyl)pyrimidin-2-yl]-N-[(8R*,9R*)-3,8,9-trimethyl-7,10- |
| 338 | dioxa-3,4,12,15-tetraazatetracyclo[9.7.0.0\hat{ }{2,6}.0\hat{ }{12,16}]octadeca- |
| | 1(11),2(6),4,13,15,17-hexaen-14-yl]cyclopropane-1-carboxamide (stereoisomer 1) |
| I- | (1S,2R)-2-[5-(difluoromethyl)pyrimidin-2-yl]-N-[(8R*,9R*)-3,8,9-trimethyl-7,10- |
| 339 | dioxa-3,4,12,15-tetraazatetracyclo[9.7.0.0\hat{ }{2,6}.0\hat{ }{12,16}]octadeca- |
| | 1(11),2(6),4,13,15,17-hexaen-14-yl]cyclopropane-1-carboxamide (stereoisomer 2) |
| I- | (1S,2R)-N-[(9R)-3,9-dimethyl-7,10-dioxa-3,4,12,15- |
| 340 | tetraazatetracyclo[9.7.0.0\hat{ }{2,6}.0\hat{ }{12,16}]octadeca-1(11),2(6),4,13,15,17-hexaen- |
| | 14-yl]-2-(5-methylpyrimidin-2-yl)cyclopropane-1-carboxamide |
| I- | rel-3-[(1R)-1-(5-methyl-1,3-thiazol-2-yl)ethyl]-1-{3-methyl-7,10-dioxa-3,4,15,16- |
| 341 | tetraazatetracyclo[9.7.0.0\hat{ }{2,6}.0\hat{ }{12,16}]octadeca-1(11),2(6),4,12,14,17-hexaen- |
| | 14-yl}urea (stereoisomer 2) |
| I- | N-[(9R)-3,9-dimethyl-7,10-dioxa-3,4,15,16- |
| 432 | tetraazatetracyclo[9.7.0.0\hat{ }{2,6}.0\hat{ }{12,16}]octadeca-1(11),2(6),4,12,14,17-hexaen- |
| | 14-yl]-2-{5-[(1-methyl-1H-pyrazol-4-yl)methyl]pyridin-2-yl}acetamide |
| I- | rel-3-[(1R)-1-(5-methyl-1,3-thiazol-2-yl)ethyl]-1-{3-methyl-7,10-dioxa-3,4,15,16- |
| 343 | tetraazatetracyclo[9.7.0.0\hat{ }{2,6}.0\hat{ }{12,16}]octadeca-1(11),2(6),4,12,14,17-hexaen- |
| | 14-yl}urea (stereoisomer 1) |
| I- | rel-3-[(1R)-1-(1-methyl-1H-pyrazol-3-yl)ethyl]-1-{3-methyl-7,10-dioxa-3,4,15,16- |
| 344 | tetraazatetracyclo[9.7.0.0\hat{ }{2,6}.0\hat{ }{12,16}]octadeca-1(11),2(6),4,12,14,17-hexaen- |
| | 14-yl}urea (stereoisomer 1) |
| I- | rel-3-[(1R)-1-(1-methyl-1H-pyrazol-3-yl)ethyl]-1-{3-methyl-7,10-dioxa-3,4,15,16- |
| 345 | tetraazatetracyclo[9.7.0.0\hat{ }{2,6}.0\hat{ }{12,16}]octadeca-1(11),2(6),4,12,14,17-hexaen- |
| | 14-yl}urea (stereoisomer 2) |
| I- | rel-1-[(1R)-1-[5-(difluoromethoxy)pyrimidin-2-yl]ethyl]-3-{3-methyl-7,10-dioxa- |
| 346 | 3,4,15,16-tetraazatetracyclo[9.7.0.0\hat{ }{2,6}.0\hat{ }{12,16}]octadeca-1(11),2(6),4,12,14,17- |
| | hexaen-14-yl}urea |
| I- | (1S,2R)-N-{3-methyl-7,10-dioxa-3,4,15,16- |
| 347 | tetraazatetracyclo[9.7.0.0\hat{ }{2,6}.0\hat{ }{12,16}]octadeca-1(11),2(6),4,12,14,17-hexaen- |
| | 14-yl}-2-[5-(pentafluoro-lambda6-sulfanyl)pyridin-2-yl]cyclopropane-1-carboxamide |
| I- | rel-1-[(1R)-2-methoxy-1-(5-methylpyrimidin-2-yl)ethyl]-3-{3-methyl-7,10-dioxa- |
| 348 | 3,4,15,16-tetraazatetracyclo[9.7.0.0\hat{ }{2,6}.0\hat{ }{12,16}]octadeca-1(11),2(6),4,12,14,17- |
| | hexaen-14-yl}urea (stereoisomer 1) |
| I- | rel-1-[(1R)-2-methoxy-1-(5-methylpyrimidin-2-yl)ethyl]-3-{3-methyl-7,10-dioxa- |
| 349 | 3,4,15,16-tetraazatetracyclo[9.7.0.0\hat{ }{2,6}.0\hat{ }{12,16}]octadeca-1(11),2(6),4,12,14,17- |
| | hexaen-14-yl}urea (stereoisomer 2) |
| I- | 2-[5-(propan-2-yl)pyrimidin-2-yl]-N-[(8R,9R)-3,8,9-trimethyl-7,10-dioxa-3,4,15,16- |
| 350 | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-(2-methyl-1,3-oxazol-4-yl)-N-[(8R,9R)-3,8,9-trimethyl-7,10-dioxa-3,4,15,16- |
| 351 | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | rel-1-[(1R)-3,3-difluoro-1-(5-methylpyrimidin-2-yl)propyl]-3-{3-methyl-7,10-dioxa- |
| 352 | 3,4,15,16-tetraazatetracyclo[9.7.0.0\hat{ }{2,6}.0\hat{ }{12,16}]octadeca-1(11),2(6),4,12,14,17- |
| | hexaen-14-yl}urea (stereoisomer 2) |
| I- | rel-1-[(1R)-3,3-difluoro-1-(5-methylpyrimidin-2-yl)propyl]-3-{3-methyl-7,10-dioxa- |
| 353 | 3,4,15,16-tetraazatetracyclo[9.7.0.0\hat{ }{2,6}.0\hat{ }{12,16}]octadeca-1(11),2(6),4,12,14,17- |
| | hexaen-14-yl}urea (stereoisomer 1) |
| I- | N-[(9R)-3,9-dimethyl-7,10-dioxa-3,4,15,16- |
| 354 | tetraazatetracyclo[9.7.0.0\hat{ }{2,6}.0\hat{ }{12,16}]octadeca-1(11),2(6),4,12,14,17-hexaen- |
| | 14-yl]-2-{5-[(1R*)-1-(1-methyl-1H-pyrazol-4-yl)ethyl]pyridin-2-yl}acetamide |
| | (stereoisomer 1) |
| I- | N-[(9R)-3,9-dimethyl-7,10-dioxa-3,4,15,16- |
| 355 | tetraazatetracyclo[9.7.0.0\hat{ }{2,6}.0\hat{ }{12,16}]octadeca-1(11),2(6),4,12,14,17-hexaen- |
| | 14-yl]-2-{5-[(1R*)-1-(1-methyl-1H-pyrazol-4-yl)ethyl]pyridin-2-yl}acetamide |
| | (stereoisomer 2) |
| I- | 2-[5-(2-hydroxypropan-2-yl)pyridin-2-yl]-N-[(8R,9R)-3,8,9-trimethyl-7,10-dioxa- |
| 356 | 3,4,15,16-tetraazatetracyclo[9.7.0.0\hat{ }{2,6}.0\hat{ }{12,16}]octadeca-1(11),2(6),4,12,14,17- |
| | hexaen-14-yl]acetamide |
| I- | 2-{5H,6H,8H-imidazo[2,1-c][1,4]oxazin-2-yl}-N-[(8R,9R)-3,8,9-trimethyl-7,10- |
| 357 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide |
| I- | 2-{5-[1-(difluoromethyl)cyclopropyl]pyridin-2-yl}-N-[(9R)-3,9-dimethyl-7,10-dioxa- |
| 358 | 3,4,15,16-tetraazatetracyclo[9.7.0.0\hat{ }{2,6}.0\hat{ }{12,16}]octadeca-1(11),2(6),4,12,14,17- |
| | hexaen-14-yl]acetamide |
| I- | 2-[1-(oxetan-3-yl)-1H-pyrazol-3-yl]-N-[(8R,9R)-3,8,9-trimethyl-7,10-dioxa- |
| 359 | 3,4,15,16-tetraazatetracyclo[9.7.0.0\hat{ }{2,6}.0\hat{ }{12,16}]octadeca-1(11),2(6),4,12,14,17- |
| | hexaen-14-yl]acetamide |
| I- | N-[(9R)-3,9-dimethyl-7,10-dioxa-3,4,15,16- |
| 360 | tetraazatetracyclo[9.7.0.0\hat{ }{2,6}.0\hat{ }{12,16}]octadeca-1(11),2(6),4,12,14,17-hexaen- |
| | 14-yl]-2-{5-[(1R*)-1-(morpholin-4-yl)ethyl]pyridin-2-yl}acetamide (stereoisomer 1) |
| I- | N-[(9R)-3,9-dimethyl-7,10-dioxa-3,4,15,16- |
| 361 | tetraazatetracyclo[9.7.0.0\hat{ }{2,6}.0\hat{ }{12,16}]octadeca-1(11),2(6),4,12,14,17-hexaen- |
| | 14-yl]-2-{5-[(1R*)-1-(morpholin-4-yl)ethyl]pyridin-2-yl}acetamide (stereoisomer 2) |
| I- | 2-{1-[(oxetan-3-yl)methyl]-1H-pyrazol-5-yl}-N-[(8R,9R)-3,8,9-trimethyl-7,10-dioxa- |
| 362 | 3,4,15,16-tetraazatetracyclo[9.7.0.0\hat{ }{2,6}.0\hat{ }{12,16}]octadeca-1(11),2(6),4,12,14,17- |
| | hexaen-14-yl]acetamide |
| I- | 2-(1,2-dimethyl-1H-imidazol-4-yl)-N-[(9R)-3,9-dimethyl-7,10-dioxa-3,4,15,16- |
| 363 | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-[4-(2-hydroxypropan-2-yl)-1,3-thiazol-2-yl]-N-[(8R,9R)-3,8,9-trimethyl-7,10- |
| 364 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide |
| I- | 2-{1-[(oxetan-3-yl)methyl]-1H-pyrazol-3-yl}-N-[(8R,9R)-3,8,9-trimethyl-7,10-dioxa- |
| 365 | 3,4,15,16-tetraazatetracyclo[9.7.0.0\hat{ }{2,6}.0\hat{ }{12,16}]octadeca-1(11),2(6),4,12,14,17- |
| | hexaen-14-yl]acetamide |
| I- | 2-[1-methyl-5-(oxan-4-yl)-1H-pyrazol-3-yl]-N-[(8R,9R)-3,8,9-trimethyl-7,10-dioxa- |
| 366 | 3,4,15,16-tetraazatetracyclo[9.7.0.0\hat{ }{2,6}.0\hat{ }{12,16}]octadeca-1(11),2(6),4,12,14,17- |
| | hexaen-14-yl]acetamide |
| I- | 2-(5-methyl-1,2,4-oxadiazol-3-yl)-N-[(8R,9R)-3,8,9-trimethyl-7,10-dioxa-3,4,15,16- |
| 367 | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-[5-(2-hydroxypropan-2-yl)pyrazin-2-yl]-N-[(8R,9R)-3,8,9-trimethyl-7,10-dioxa- |
| 368 | 3,4,15,16-tetraazatetracyclo[9.7.0.0\hat{ }{2,6}.0\hat{ }{12,16}]octadeca-1(11),2(6),4,12,14,17- |
| | hexaen-14-yl]acetamide |
| I- | 2-{5-[(2R*)-2-hydroxy-1-methoxypropan-2-yl]pyridin-2-yl}-N-[(8R,9R)-3,8,9- |
| 369 | trimethyl-7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide (stereoisomer 2) |
| I- | 2-{5-[(2R*)-2-hydroxy-1-methoxypropan-2-yl]pyridin-2-yl}-N-[(8R,9R)-3,8,9- |
| 370 | trimethyl-7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide (stereoisomer 1) |
| I- | 2-{5-methyl-4H,5H,6H,7H-[1,3]thiazolo[4,5-c]pyridin-2-yl}-N-[(8R,9R)-3,8,9- |
| 371 | trimethyl-7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide |
| I- | 2-[(5R)-5-methyl-5H,6H,8H-imidazo[2,1-c][1,4]oxazin-2-yl]-N-[(8R,9R)-3,8,9- |
| 372 | trimethyl-7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide |
| I- | 2-(5-{3-hydroxybicyclo[1.1.1]pentan-1-yl}pyridin-2-yl)-N-[(8R,9R)-3,8,9-trimethyl- |
| 373 | 7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl](2H2)acetamide |
| I- | 2-{5H,6H,8H,9H-imidazo[1,2-d][1,4]oxazepin-2-yl}-N-[(8R,9R)-3,8,9-trimethyl- |
| 374 | 7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide |
| I- | 2-{7-[(2R*)-1,1-difluoropropan-2-yl]-5H,6H,7H,8H-imidazo[1,2-a]pyrazin-2-yl}-N- |
| 375 | [(8R,9R)-3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide (stereoisomer 2) |
| I- | 2-{7-[(2R*)-1,1-difluoropropan-2-yl]-5H,6H,7H,8H-imidazo[1,2-a]pyrazin-2-yl}-N- |
| 376 | [(8R,9R)-3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide (stereoisomer 1) |
| I- | 2-[5-(difluoromethyl)-1-methyl-1H-1,2,4-triazol-3-yl]-N-[(8R,9R)-3,8,9-trimethyl- |
| 377 | 7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide |
| I- | 2-{1-methyl-1H-pyrazolo[4,3-b]pyridin-5-yl}-N-[(8R,9R)-3,8,9-trimethyl-7,10-dioxa- |
| 378 | 3,4,15,16-tetraazatetracyclo[9.7.0.0\hat{ }{2,6}.0\hat{ }{12,16}]octadeca-1(11),2(6),4,12,14,17- |
| | hexaen-14-yl]acetamide |
| I- | 2-{7-cyclopropyl-5H,6H,7H,8H-imidazo[1,2-a]pyrazin-2-yl}-N-[(8R,9R)-3,8,9- |
| 379 | trimethyl-7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide |
| I- | 2-{5-[(2R*)-2-methyl-1,4-dioxan-2-yl]pyrimidin-2-yl}-N-[(8R,9R)-3,8,9-trimethyl- |
| 380 | 7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide (stereoisomer 2) |
| I- | 2-{5-[(2R*)-2-methyl-1,4-dioxan-2-yl]pyrimidin-2-yl}-N-[(8R,9R)-3,8,9-trimethyl- |
| 381 | 7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide (stereoisomer 1) |
| I- | 2-[(3R*)-3-(difluoromethyl)-1H,3H-furo[3,4-c]pyridin-6-yl]-N-[(8R,9R)-3,8,9- |
| 382 | trimethyl-7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide (stereoisomer 2) |
| I- | 2-[(3R*)-3-(difluoromethyl)-1H,3H-furo[3,4-c]pyridin-6-yl]-N-[(8R,9R)-3,8,9- |
| 383 | trimethyl-7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide (stereoisomer 1) |
| I- | 2-[5-methyl-1-(oxetan-3-yl)-1H-imidazol-4-yl]-N-[(8R,9R)-3,8,9-trimethyl-7,10- |
| 384 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide |
| I- | 2-[(6R*)-6-methyl-5H,6H,8H-imidazo[2,1-c][1,4]oxazin-2-yl]-N-[(8R,9R)-3,8,9- |
| 385 | trimethyl-7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide (stereoisomer 2) |
| I- | 2-[(6R*)-6-methyl-5H,6H,8H-imidazo[2,1-c][1,4]oxazin-2-yl]-N-[(8R,9R)-3,8,9- |
| 386 | trimethyl-7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide (stereoisomer 1) |
| I- | 2-[1-(2,2-difluoroethyl)-2-methyl-1H-imidazol-4-yl]-N-[(8R,9R)-3,8,9-trimethyl- |
| 387 | 7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide |
| I- | 2-{5-[(2R*)-2-methyloxetan-2-yl]pyrimidin-2-yl}-N-[(8R,9R)-3,8,9-trimethyl-7,10- |
| 388 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide (stereoisomer 2) |
| I- | 2-{5-[(2R*)-2-methyloxetan-2-yl]pyrimidin-2-yl}-N-[(8R,9R)-3,8,9-trimethyl-7,10- |
| 389 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide (stereoisomer 1) |
| I- | 2-[6-oxo-5-(propan-2-yl)-4H,5H,6H,7H-pyrazolo[1,5-a]pyrazin-2-yl]-N-[(8R,9R)- |
| 390 | 3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-{5′,8′-dihydrospiro[cyclopropane-1,6′-imidazo[2,1-c][1,4]oxazin]-2′-yl}-N- |
| 391 | [(8R,9R)-3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-{5-methyl-4H,5H,6H,7H-pyrazolo[1,5-a]pyrazin-2-yl}-N-[(8R,9R)-3,8,9-trimethyl- |
| 392 | 7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide |
| I- | (1R*,2R*)-N-[(9R)-3,9-dimethyl-7,10-dioxa-3,4,15,16- |
| 393 | tetraazatetracyclo[9.7.0.0\hat{ }{2,6}.0\hat{ }{12,16}]octadeca-1(11),2(6),4,12,14,17-hexaen- |
| | 14-yl]-2-(5-methylpyrimidin-2-yl)cyclobutane-1-carboxamide (stereoisomer 2) |
| I- | (1R*,2R*)-N-[(9R)-3,9-dimethyl-7,10-dioxa-3,4,15,16- |
| 394 | tetraazatetracyclo[9.7.0.0\hat{ }{2,6}.0\hat{ }{12,16}]octadeca-1(11),2(6),4,12,14,17-hexaen- |
| | 14-yl]-2-(5-methylpyrimidin-2-yl)cyclobutane-1-carboxamide (stereoisomer 1) |
| I- | 2-[(5S)-5-methyl-5H,6H,8H-imidazo[2,1-c][1,4]oxazin-2-yl]-N-[(8R,9R)-3,8,9- |
| 395 | trimethyl-7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide |
| I- | 2-{5-[1-(difluoromethyl)cyclopropyl]pyrimidin-2-yl}-N-[(8R,9R)-3,8,9-trimethyl- |
| 396 | 7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide |
| I- | 2-{5-[(2R*)-1,1-difluoropropan-2-yl]pyrimidin-2-yl}-N-[(8R,9R)-3,8,9-trimethyl- |
| 397 | 7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide (stereoisomer 2) |
| I- | 2-{5-[(2R*)-1,1-difluoropropan-2-yl]pyrimidin-2-yl}-N-[(8R,9R)-3,8,9-trimethyl- |
| 398 | 7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide (stereoisomer 1) |
| I- | 2-{imidazo[1,2-a]pyrazin-2-yl}-N-[(8R,9R)-3,8,9-trimethyl-7,10-dioxa-3,4,15,16- |
| 399 | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-(1,2-dimethyl-1H-imidazol-4-yl)-N-[(8R,9R)-3,8,9-trimethyl-7,10-dioxa-3,4,15,16- |
| 400 | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-{5-[(2R*)-2-methyloxolan-2-yl]pyrimidin-2-yl}-N-[(8R,9R)-3,8,9-trimethyl-7,10- |
| 401 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide (stereoisomer 2) |
| I- | 2-{5-[(2R*)-2-methyloxolan-2-yl]pyrimidin-2-yl}-N-[(8R,9R)-3,8,9-trimethyl-7,10- |
| 402 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide (stereoisomer 1) |
| I- | 2-{5-[(2R*)-2-methyl-1,4-dioxan-2-yl]pyridin-2-yl}-N-[(8R,9R)-3,8,9-trimethyl- |
| 403 | 7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide (stereoisomer 2) |
| I- | 2-{5-[(2R*)-2-methyl-1,4-dioxan-2-yl]pyridin-2-yl}-N-[(8R,9R)-3,8,9-trimethyl- |
| 404 | 7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide (stereoisomer 1) |
| I- | 2-(5-{3-fluorobicyclo[1.1.1]pentan-1-yl}pyrimidin-2-yl)-N-[(8R,9R)-3,8,9-trimethyl- |
| 405 | 7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide |
| I- | 2-[5-(1-hydroxycyclopropyl)pyridin-2-yl]-N-[(8R,9R)-3,8,9-trimethyl-7,10-dioxa- |
| 406 | 3,4,15,16-tetraazatetracyclo[9.7.0.0\hat{ }{2,6}.0\hat{ }{12,16}]octadeca-1(11),2(6),4,12,14,17- |
| | hexaen-14-yl]acetamide |
| I- | 2-{5-[(1R*)-1-cyclopropyl-1-hydroxyethyl]-1-methyl-1H-pyrazol-3-yl}-N-[(8R,9R)- |
| 407 | 3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide (stereoisomer 2) |
| I- | 2-{5-[(1R*)-1-cyclopropyl-1-hydroxyethyl]-1-methyl-1H-pyrazol-3-yl}-N-[(8R,9R)- |
| 408 | 3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide (stereoisomer 1) |
| I- | 2-[5-methyl-1-(propan-2-yl)-1H-1,2,4-triazol-3-yl]-N-[(8R,9R)-3,8,9-trimethyl-7,10- |
| 409 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide |
| I- | N-[(9R)-3,9-dimethyl-7,10-dioxa-3,4,15,16- |
| 410 | tetraazatetracyclo[9.7.0.0\hat{ }{2,6}.0\hat{ }{12,16}]octadeca-1(11),2(6),4,12,14,17-hexaen- |
| | 14-yl]-2-(1-{6-fluoroimidazo[1,2-a]pyridin-3-yl}-5-methyl-1H-pyrazol-3- |
| | yl)acetamide |
| I- | 2-[5-(4-hydroxyoxan-4-yl)pyridin-2-yl]-N-[(8R,9R)-3,8,9-trimethyl-7,10-dioxa- |
| 411 | 3,4,15,16-tetraazatetracyclo[9.7.0.0\hat{ }{2,6}.0\hat{ }{12,16}]octadeca-1(11),2(6),4,12,14,17- |
| | hexaen-14-yl]acetamide |
| I- | 2-{5-[(2R*)-1,1-difluoro-2-hydroxypropan-2-yl]pyridin-2-yl}-N-[(8R,9R)-3,8,9- |
| 412 | trimethyl-7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide (stereoisomer 2) |
| I- | 2-{5-[(2R*)-1,1-difluoro-2-hydroxypropan-2-yl]pyridin-2-yl}-N-[(8R,9R)-3,8,9- |
| 413 | trimethyl-7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide (stereoisomer 1) |
| I- | N-[(9R)-3,9-dimethyl-10-oxa-3,4,15,16- |
| 414 | tetraazatetracyclo[9.7.0.0\hat{ }{2,6}.0\hat{ }{12,16}]octadeca-1(11),2(6),4,12,14,17-hexaen- |
| | 14-yl]-2-[5-(2-hydroxypropan-2-yl)pyridin-2-yl]acetamide |
| I- | N-{9,9-difluoro-3-methyl-7,11-dioxa-3,4,16,17- |
| 415 | tetraazatetracyclo[10.7.0.0\hat{ }{2,6}.0\hat{ }{13,17}]nonadeca-1(12),2(6),4,13,15,18-hexaen- |
| | 15-yl}-2-[5-(2-hydroxypropan-2-yl)pyridin-2-yl]acetamide |
| I- | 2-[(7R*)-7-hydroxy-7-methyl-5H,6H,7H-cyclopenta[c]pyridin-3-yl]-N-[(8R,9R)- |
| 416 | 3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide (stereoisomer 2) |
| I- | 2-[(7R*)-7-hydroxy-7-methyl-5H,6H,7H-cyclopenta[c]pyridin-3-yl]-N-[(8R,9R)- |
| 417 | 3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide (stereoisomer 1) |
| I- | 2-{5-[(1R*)-1-hydroxyethyl]pyridin-2-yl}-N-[(8R,9R)-3,8,9-trimethyl-7,10-dioxa- |
| 418 | 3,4,15,16-tetraazatetracyclo[9.7.0.0\hat{ }{2,6}.0\hat{ }{12,16}]octadeca-1(11),2(6),4,12,14,17- |
| | hexaen-14-yl]acetamide (stereoisomer 2) |
| I- | 2-{5-[(1R*)-1-hydroxyethyl]pyridin-2-yl}-N-[(8R,9R)-3,8,9-trimethyl-7,10-dioxa- |
| 419 | 3,4,15,16-tetraazatetracyclo[9.7.0.0\hat{ }{2,6}.0\hat{ }{12,16}]octadeca-1(11),2(6),4,12,14,17- |
| | hexaen-14-yl]acetamide (stereoisomer 1) |
| I- | 2-[5-(4-cyanooxan-4-yl)pyridin-2-yl]-N-[(8R,9R)-3,8,9-trimethyl-7,10-dioxa- |
| 420 | 3,4,15,16-tetraazatetracyclo[9.7.0.0\hat{ }{2,6}.0\hat{ }{12,16}]octadeca-1(11),2(6),4,12,14,17- |
| | hexaen-14-yl]acetamide |
| I- | 2-[5-(1,4-dimethyl-1H-pyrazol-5-yl)pyridin-2-yl]-N-[(8R,9R)-3,8,9-trimethyl-7,10- |
| 421 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide |
| I- | 2-{7-methyl-5H,6H,7H,8H-imidazo[1,2-a]pyrazin-2-yl}-N-[(8R,9R)-3,8,9-trimethyl- |
| 422 | 7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide |
| I- | 2-{5-[(1R*)-2,2,2-trifluoro-1-hydroxyethyl]pyridin-2-yl}-N-[(8R,9R)-3,8,9-trimethyl- |
| 423 | 7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide (stereoisomer 2) |
| I- | 2-{5-[(1R*)-2,2,2-trifluoro-1-hydroxyethyl]pyridin-2-yl}-N-[(8R,9R)-3,8,9-trimethyl- |
| 424 | 7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide (stereoisomer 1) |
| I- | 2-[5-(difluoromethoxy)pyridin-2-yl]-N-[(8R,9R)-3,8,9-trimethyl-7,10-dioxa- |
| 425 | 3,4,15,16-tetraazatetracyclo[9.7.0.0\hat{ }{2,6}.0\hat{ }{12,16}]octadeca-1(11),2(6),4,12,14,17- |
| | hexaen-14-yl]acetamide |
| I- | 2-[5-(1-hydroxycyclobutyl)pyridin-2-yl]-N-[(8R,9R)-3,8,9-trimethyl-7,10-dioxa- |
| 426 | 3,4,15,16-tetraazatetracyclo[9.7.0.0\hat{ }{2,6}.0\hat{ }{12,16}]octadeca-1(11),2(6),4,12,14,17- |
| | hexaen-14-yl]acetamide |
| I- | N-[(9R)-3,9-dimethyl-10-oxa-3,4,15,16- |
| 427 | tetraazatetracyclo[9.7.0.0\hat{ }{2,6}.0\hat{ }{12,16}]octadeca-1(11),2(6),4,12,14,17-hexaen- |
| | 14-yl]-2-[5-(1-hydroxycyclopropyl)pyridin-2-yl]acetamide |
| I- | 2-{5H,6H,8H-[1,2,4]triazolo[3,2-c][1,4]oxazin-2-yl}-N-[(8R,9R)-3,8,9-trimethyl- |
| 428 | 7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide |
| I- | 2-{5′,7′-dihydrospiro[cyclopropane-1,6′-pyrrolo[1,2-a]imidazol]-2′-yl}-N-[(8R,9R)- |
| 429 | 3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-[(8R*)-8-methyl-5H,6H,8H-imidazo[2,1-c][1,4]oxazin-2-yl]-N-[(8R,9R)-3,8,9- |
| 430 | trimethyl-7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide (stereoisomer 1) |
| I- | 2-[(8R*)-8-methyl-5H,6H,8H-imidazo[2,1-c][1,4]oxazin-2-yl]-N-[(8R,9R)-3,8,9- |
| 431 | trimethyl-7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide (stereoisomer 2) |
| I- | 2-{5-[(4s)-2-oxabicyclo[2.1.1]hexan-4-yl]pyrimidin-2-yl}-N-[(8R,9R)-3,8,9- |
| 432 | trimethyl-7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide |
| I- | 2-{7-cyclopropyl-5H,6H,7H,8H-imidazo[1,2-a]pyrazin-2-yl}-N-[(9R)-3,9-dimethyl- |
| 433 | 7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide |
| I- | 2-[5-methyl-1-(oxetan-3-yl)-1H-1,2,4-triazol-3-yl]-N-[(8R,9R)-3,8,9-trimethyl-7,10- |
| 434 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide |
| I- | 2-{5′,8′-dihydrospiro[cyclopropane-1,6′-imidazo[2,1-c][1,4]oxazin]-2′-yl}-N-[(9R)- |
| 435 | 3,9-dimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-{1-[(1R*)-2,2-difluorocyclopropyl]-2-methyl-1H-imidazol-4-yl}-N-[(8R,9R)-3,8,9- |
| 436 | trimethyl-7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide (stereoisomer 1) |
| I- | 2-{1-[(1R*)-2,2-difluorocyclopropyl]-2-methyl-1H-imidazol-4-yl}-N-[(8R,9R)-3,8,9- |
| 437 | trimethyl-7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide (stereoisomer 2) |
| I- | (1S,2R)-2-[5-(difluoromethyl)pyridin-2-yl]-N-[(8R*)-3-methyl-8- |
| 438 | [(methylsulfanyl)methyl]-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]cyclopropane-1-carboxamide (stereoisomer 1) |
| I- | (1S,2R)-2-[5-(difluoromethyl)pyridin-2-yl]-N-[(8R*)-3-methyl-8- |
| 439 | [(methylsulfanyl)methyl]-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]cyclopropane-1-carboxamide (stereoisomer 2) |
| I- | N-[(9R)-3,9-dimethyl-10-oxa-3,4,15,16- |
| 440 | tetraazatetracyclo[9.7.0.0\hat{ }{2,6}.0\hat{ }{12,16}]octadeca-1(11),2(6),4,12,14,17-hexaen- |
| | 14-yl]-2-{7-methyl-5H,6H,7H,8H-imidazo[1,2-a]pyrazin-2-yl}acetamide |
| I- | N-[(9R)-3,9-dimethyl-10-oxa-3,4,15,16- |
| 441 | tetraazatetracyclo[9.7.0.0\hat{ }{2,6}.0\hat{ }{12,16}]octadeca-1(11),2(6),4,12,14,17-hexaen- |
| | 14-yl]-2-{5H,6H,8H,9H-imidazo[1,2-d][1,4]oxazepin-2-yl}acetamide |
| I- | 2-{2-methyl-2H-pyrazolo[4,3-b]pyridin-5-yl}-N-[(8R,9R)-3,8,9-trimethyl-7,10-dioxa- |
| 442 | 3,4,15,16-tetraazatetracyclo[9.7.0.0\hat{ }{2,6}.0\hat{ }{12,16}]octadeca-1(11),2(6),4,12,14,17- |
| | hexaen-14-yl]acetamide |
| I- | 2-(5-methyl-6-oxo-5,6,7,8-tetrahydro-1,5-naphthyridin-2-yl)-N-[(8R,9R)-3,8,9- |
| 443 | trimethyl-7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide |
| I- | (1S,2R)-N-[(9R*)-3-methyl-9-(methylsulfanyl)-7,11-dioxa-3,4,16,17- |
| 444 | tetraazatetracyclo[10.7.0.0\hat{ }{2,6}.0\hat{ }{13,17}]nonadeca-1(12),2(6),4,13,15,18-hexaen- |
| | 15-yl]-2-(5-methylpyrimidin-2-yl)cyclopropane-1-carboxamide (stereoisomer 2) |
| I- | (1S,2R)-N-[(9R*)-3-methyl-9-(methylsulfanyl)-7,11-dioxa-3,4,16,17- |
| 445 | tetraazatetracyclo[10.7.0.0\hat{ }{2,6}.0\hat{ }{13,17}]nonadeca-1(12),2(6),4,13,15,18-hexaen- |
| | 15-yl]-2-(5-methylpyrimidin-2-yl)cyclopropane-1-carboxamide (stereoisomer 1) |
| I- | 2-{7′-methyl-7′,8′-dihydro-5′H-spiro[cyclopropane-1,6′-imidazo[1,2-a]pyrazin]-2′-yl}- |
| 446 | N-[(8R,9R)-3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-[5-(2-hydroxypropan-2-yl)-1,3-oxazol-2-yl]-N-[(8R,9R)-3,8,9-trimethyl-7,10-dioxa- |
| 447 | 3,4,15,16-tetraazatetracyclo[9.7.0.0\hat{ }{2,6}.0\hat{ }{12,16}]octadeca-1(11),2(6),4,12,14,17- |
| | hexaen-14-yl]acetamide |
| I- | N-[(9R)-3,9-dimethyl-10-oxa-3,4,15,16- |
| 448 | tetraazatetracyclo[9.7.0.0\hat{ }{2,6}.0\hat{ }{12,16}]octadeca-1(11),2(6),4,12,14,17-hexaen- |
| | 14-yl]-2-[7-(oxetan-3-yl)-5H,6H,7H,8H-imidazo[1,2-a]pyrazin-2-yl]acetamide |
| I- | 2-[5-(cyclopropanesulfonyl)pyridin-2-yl]-N-[(8R,9R)-3,8,9-trimethyl-7,10-dioxa- |
| 449 | 3,4,15,16-tetraazatetracyclo[9.7.0.0\hat{ }{2,6}.0\hat{ }{12,16}]octadeca-1(11),2(6),4,12,14,17- |
| | hexaen-14-yl]acetamide |
| I- | 2-{7-ethyl-5H,6H,7H,8H-imidazo[1,2-a]pyrazin-2-yl}-N-[(8R,9R)-3,8,9-trimethyl- |
| 450 | 7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide |
| I- | (1S,2R)-N-{3-methyl-11-oxa-7-thia-3,4,16,17- |
| 451 | tetraazatetracyclo[10.7.0.0\hat{ }{2,6}.0\hat{ }{13,17}]nonadeca-1(12),2(6),4,13,15,18-hexaen- |
| | 15-yl}-2-(5-methylpyrimidin-2-yl)cyclopropane-1-carboxamide |
| I- | 2-[8-oxo-7-(propan-2-yl)-5H,6H,7H,8H-imidazo[1,2-a]pyrazin-2-yl]-N-[(8R,9R)- |
| 452 | 3,8,9-trimethyl-7,10-dioxa-3,4,15,16-tetraazatetracyclo[9.7.0.0\hat{ }{2,6}. |
| | 0\hat{ }{12,16}]octadeca-1(11),2(6),4,12,14,17-hexaen-14-yl]acetamide |
| I- | (1S,2R)-N-{3-methyl-7-oxa-11-thia-3,4,16,17- |
| 453 | tetraazatetracyclo[10.7.0.0\hat{ }{2,6}.0\hat{ }{13,17}]nonadeca-1(12),2(6),4,13,15,18-hexaen- |
| | 15-yl}-2-(5-methylpyrimidin-2-yl)cyclopropane-1-carboxamide |
| I- | 2-{7-[(4-methyl-1,3-oxazol-2-yl)methyl]-5H,6H,7H,8H-imidazo[1,2-a]pyrazin-2-yl}- |
| 454 | N-[(8R,9R)-3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-{7-cyclopropanecarbonyl-5H,6H,7H,8H-imidazo[1,2-a]pyrazin-2-yl}-N-[(8R,9R)- |
| 455 | 3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-{5-[(3S,3aR,6aR)-hexahydrofuro[3,2-b]furan-3-yl]pyrimidin-2-yl}-N-[(8R,9R)- |
| 456 | 3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-{5-methyl-4H,5H,6H,7H-[1,3]oxazolo[4,5-c]pyridin-2-yl}-N-[(8R,9R)-3,8,9- |
| 457 | trimethyl-7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide |
| I- | 2-[(2S)-5′,7′-dihydrospiro[oxetane-2,6′-pyrrolo[1,2-a]imidazol]-2′-yl]-N-[(8R,9R)- |
| 458 | 3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-[(2R)-5′,7′-dihydrospiro[oxetane-2,6′-pyrrolo[1,2-a]imidazol]-2′-yl]-N-[(8R,9R)- |
| 459 | 3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-{5,8-dihydrospiro[1,2,4]triazolo[3,2-c][1,4]oxazine-6,1′-cyclopropan]-2-yl}-N- |
| 460 | [(8R,9R)-3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-{7-methyl-7,8-dihydro-5H-spiro[1,2,4]triazolo[1,5-a]pyrazine-6,1′-cyclopropan]-2- |
| 461 | yl}-N-[(8R,9R)-3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-[(3S,5S)-1,8-diazatricyclo[5.3.0.0\hat{ }{3,5}]deca-7,9-dien-9-yl]-N-[(8R,9R)-3,8,9- |
| 462 | trimethyl-7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide |
| I- | 2-{5-[(3R,3aR,6aR)-hexahydrofuro[3,2-b]furan-3-yl]pyrimidin-2-yl}-N-[(8R,9R)- |
| 463 | 3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-{7-[(1R)-2,2-difluorocyclopropyl]-5H,6H,7H,8H-imidazo[1,2-a]pyrazin-2-yl}-N- |
| 464 | [(8R,9R)-3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-{7-cyclopropyl-5H,6H,7H,8H-[1,2,4]triazolo[1,5-a]pyrazin-2-yl}-N-[(8R,9R)- |
| 465 | 3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-[(5R)-5-hydroxy-5-methyl-4H,5H,6H-cyclopenta[d][1,3]oxazol-2-yl]-N-[(8R,9R)- |
| 466 | 3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-{5H,6H,8H-imidazo[4,3-c][1,4]oxazin-1-yl}-N-[(8R,9R)-3,8,9-trimethyl-7,10- |
| 467 | dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide |
| I- | 2-[(5R)-5-hydroxy-5-methyl-4H,5H,6H-cyclopenta[d][1,3]thiazol-2-yl]-N-[(8R,9R)- |
| 468 | 3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-(1,3-dimethyl-1H-1,2,4-triazol-5-yl)-N-[(8R,9R)-3,8,9-trimethyl-7,10-dioxa- |
| 469 | 3,4,15,16-tetraazatetracyclo[9.7.0.0\hat{ }{2,6}.0\hat{ }{12,16}]octadeca-1(11),2(6),4,12,14,17- |
| | hexaen-14-yl]acetamide |
| I- | (3S)-3-hydroxy-3-(5-methylpyrimidin-2-yl)-N-[(8R,9R)-3,8,9-trimethyl-7,10-dioxa- |
| 470 | 3,4,15,16-tetraazatetracyclo[9.7.0.0\hat{ }{2,6}.0\hat{ }{12,16}]octadeca-1(11),2(6),4,12,14,17- |
| | hexaen-14-yl]butanamide |
| I- | (3R)-3-hydroxy-3-(5-methylpyrimidin-2-yl)-N-[(8R,9R)-3,8,9-trimethyl-7,10-dioxa- |
| 471 | 3,4,15,16-tetraazatetracyclo[9.7.0.0\hat{ }{2,6}.0\hat{ }{12,16}]octadeca-1(11),2(6),4,12,14,17- |
| | hexaen-14-yl]butanamide |
| I- | 2-{1-methyl-5-[(3r)-3-hydroxy-3-methylcyclobutyl]-1H-1,2,4-triazol-3-yl}-N- |
| 472 | [(8R,9R)-3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-{1-methyl-5-[(3s)-3-hydroxy-3-methylcyclobutyl]-1H-1,2,4-triazol-3-yl}-N- |
| 473 | [(8R,9R)-3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-[(5S)-5-hydroxy-5-methyl-4,5,6,7-tetrahydro-2H-indazol-2-yl]-N-[(8R,9R)-3,8,9- |
| 474 | trimethyl-7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide |
| I- | 2-[(5R)-5-hydroxy-5-methyl-4,5,6,7-tetrahydro-2H-indazol-2-yl]-N-[(8R,9R)-3,8,9- |
| 475 | trimethyl-7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide |
| I- | 2-{5-[(3R,3aR,6R,6aR)-6-hydroxy-hexahydrofuro[3,2-b]furan-3-yl]pyrimidin-2-yl}- |
| 476 | N-[(8R,9R)-3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-{5-[(3R,3aR,6S,6aR)-6-hydroxy-hexahydrofuro[3,2-b]furan-3-yl]pyrimidin-2-yl}- |
| 477 | N-[(8R,9R)-3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-[(6R)-6-hydroxy-6-methyl-4,5,6,7-tetrahydro-1,3-benzoxazol-2-yl]-N-[(8R,9R)- |
| 478 | 3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-[(6S)-6-hydroxy-6-methyl-4,5,6,7-tetrahydro-1,3-benzoxazol-2-yl]-N-[(8R,9R)- |
| 479 | 3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-[(5S)-5-hydroxy-5-methyl-4,5,6,7-tetrahydro-2H-1,2,3-benzotriazol-2-yl]-N- |
| 480 | [(8R,9R)-3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yllacetamide |
| I- | 2-[(5R)-5-hydroxy-5-methyl-4,5,6,7-tetrahydro-2H-1,2,3-benzotriazol-2-yl]-N- |
| 481 | [(8R,9R)-3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-{7-[(1S)-2,2-difluorocyclopropyl]-5H,6H,7H,8H-[1,2,4]triazolo[1,5-a]pyrazin-2- |
| 482 | yl}-N-[(8R,9R)-3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-{7-[(1R)-2,2-difluorocyclopropyl]-5H,6H,7H,8H-[1,2,4]triazolo[1,5-a]pyrazin-2- |
| 483 | yl}-N-[(8R,9R)-3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-[(6R)-6-(difluoromethyl)-5H,6H,7H-pyrrolo[1,2-b][1,2,4]triazol-2-yl]-N-[(8R,9R)- |
| 484 | 3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-[(5S)-5-hydroxy-5-methyl-2H,4H,5H,6H-cyclopenta[c]pyrazol-2-yl]-N-[(8R,9R)- |
| 485 | 3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yllacetamide |
| I- | 2-[(5R)-5-hydroxy-5-methyl-2H,4H,5H,6H-cyclopenta[c]pyrazol-2-yl]-N-[(8R,9R)- |
| 486 | 3,8,9-trimethyl-7,10-dioxa-3,4,15,16- |
| | tetraazatetracyclo[9.7.0.0\hat{ }{2,6}.0\hat{ }{12,16}]octadeca-1(11),2(6),4,12,14,17-hexaen- |
| | 14-yl]acetamide |
| I- | 2-{3,7-dimethyl-5H,6H,7H,8H-imidazo[1,2-a]pyrazin-2-yl}-N-[(8R,9R)-3,8,9- |
| 487 | trimethyl-7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide |
| I- | 2-{7-methyl-5H,6H,7H,8H-imidazo[1,5-a]pyrazin-1-yl}-N-[(8R,9R)-3,8,9-trimethyl- |
| 488 | 7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]acetamide |
| I- | (3S)-3-hydroxy-3-{5H,6H,8H-imidazo[2,1-c][1,4]oxazin-2-yl}-N-[(8R,9R)-3,8,9- |
| 489 | trimethyl-7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]butanamide |
| I- | (3R)-3-hydroxy-3-{5H,6H,8H-imidazo[2,1-c][1,4]oxazin-2-yl}-N-[(8R,9R)-3,8,9- |
| 490 | trimethyl-7,10-dioxa-3,4,15,16-tetraazatetracyclo[9.7.0.0\hat{ }{2,6}.0\hat{ }{12,16}]octadeca- |
| | 1(11),2(6),4,12,14,17-hexaen-14-yl]butanamide |
| I- | 2-(5-hydroxy-5-methyl-5,6-dihydro-4H-cyclopenta[d]thiazol-2-yl)-N-((2R,3R)-2,3,7- |
| 491 | trimethyl-2,3-dihydro-7H-pyrazolo[1,5-a]pyrazolo[3′,4′:7,8][1,4]dioxocino[5,6- |
| | c]pyridin-12-yl)acetamide |
| I- | (R*)-3-hydroxy-3-(5-methylpyrimidin-2-yl)-N-((2R,3R)-2,3,7-trimethyl-2,3-dihydro- |
| 492 | 7H-pyrazolo[1,5-a]pyrazolo[3′,4′:7,8][1,4]dioxocino[5,6-c]pyridin-12-yl)butanamide |
| | (stereoisomer 2) |
| I- | (R*)-3-hydroxy-3-(5-methylpyrimidin-2-yl)-N-((2R,3R)-2,3,7-trimethyl-2,3-dihydro- |
| 493 | 7H-pyrazolo[1,5-a]pyrazolo[3′,4′:7,8][1,4]dioxocino[5,6-c]pyridin-12-yl)butanamide |
| | (stereoisomer 1) |
| I- | 2-(7-(2,2-difluoroethyl)-5,6,7,8-tetrahydroimidazo[1,2-a]pyrazin-2-yl)-N-((2R,3R)- |
| 494 | 2,3,7-trimethyl-2,3-dihydro-7H-pyrazolo[1,5-a]pyrazolo[3′,4′:7,8][1,4]dioxocino[5,6- |
| | c]pyridin-12-yl)acetamide |
| I- | N-(6,6-difluoro-11-methyl-6,7-dihydro-5H,11H-pyrazolo[1,5- |
| 495 | alpyrazolo[3′,4′:8,9][1,5]dioxonino[6,7-c]pyridin-2-yl)-2-(7-methyl-5,6,7,8- |
| | tetrahydroimidazo[1,2-a]pyrazin-2-yl)acetamide |
| I- | 2-(7-ethyl-5,6,7,8-tetrahydroimidazo[1,2-a]pyrazin-2-yl)-N-((2aS,13aR)-6-methyl- |
| 496 | 1,2,2a,13a-tetrahydro-6H-cyclobuta[2,3]pyrazolo[3′,4′:7,8][1,4]dioxocino[5,6- |
| | c]pyrazolo[1,5-a]pyridin-11-yl)acetamide |
| I- | N-(6,6-difluoro-11-methyl-6,7-dihydro-5H,11H-pyrazolo[1,5- |
| 497 | alpyrazolo[3′,4′:8,9][1,5]dioxonino[6,7-c]pyridin-2-yl)-2-(7-ethyl-5,6,7,8- |
| | tetrahydroimidazo[1,2-a]pyrazin-2-yl)acetamide |
| I- | 2-(5′-methyl-6′-oxo-5′,8′-dihydro-6′H-spiro[cyclopropane-1,7′-pyrido[2,3-b]pyrazin]- |
| 498 | 2′-yl)-N-((2R,3R)-2,3,7-trimethyl-2,3-dihydro-7H-pyrazolo[1,5- |
| | alpyrazolo[3′,4′:7,8][1,4]dioxocino[5,6-c]pyridin-12-yl)acetamide |
| I- | 2-(1-methyl-2-oxo-1,4-dihydro-2H-pyrido[3,2-d][1,3]oxazin-6-yl)-N-((2R,3R)-2,3,7- |
| 499 | trimethyl-2,3-dihydro-7H-pyrazolo[1,5-a]pyrazolo[3′,4′:7,8][1,4]dioxocino[5,6- |
| | c]pyridin-12-yl)acetamide |
| I- | 2-((5aR,6aR)-5a,6,6a,7-tetrahydro-5H-cyclopropa[d]imidazo[1,2-a]pyridin-2-yl)-N- |
| 500 | ((2R,3R)-2,3,7-trimethyl-2,3-dihydro-7H-pyrazolo[1,5- |
| | alpyrazolo[3′,4′:7,8][1,4]dioxocino[5,6-c]pyridin-12-yl)acetamide (stereoisomer 2) |

In some embodiments, the present invention provides a compound set forth in Table 1, above, or a pharmaceutically acceptable salt thereof. In some embodiments, the present invention provides a compound set forth in Table 1, above.

In some embodiments, the present invention provides a pharmaceutical composition comprising a compound disclosed herein (described in embodiments herein, both singly and in combination), or a pharmaceutically acceptable salt thereof, together with a pharmaceutically acceptable carrier, excipient, or diluent. For example, in some embodiments, the present invention provides a pharmaceutical composition comprising a compound of formula I as defined above, or a pharmaceutically acceptable salt thereof, together with a pharmaceutically acceptable carrier, excipient, or diluent. In some embodiments, the present invention provides a pharmaceutical composition comprising a compound of formula I as defined above, together with a pharmaceutically acceptable carrier, excipient, or diluent. In some embodiments, the present invention provides a pharmaceutical composition comprising a compound set forth in Table 1 above, or a pharmaceutically acceptable salt thereof, together with a pharmaceutically acceptable carrier, excipient, or diluent. In some embodiments, the present invention provides a pharmaceutical composition comprising a compound set forth in Table 1 above, together with a pharmaceutically acceptable carrier, excipient, or diluent.

### 4. General Methods of Providing the Present Compounds

The compounds of this invention may be prepared or isolated in general by synthetic and/or semi-synthetic methods known to those skilled in the art for analogous compounds and by methods described in detail in the Examples, herein.

### 5. Uses, Formulation and Administration

### Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. In certain embodiments, the amount of compound in compositions of this invention is such that is effective to measurably inhibit SIK2, or a mutant thereof, in a biological sample or in a patient. In certain embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitory active metabolite or residue thereof.

As used herein, the term “active metabolite or residue thereof” means that a metabolite or residue thereof is also an inhibitor of SIK2, or a mutant thereof.

The subject matter disclosed herein includes prodrugs, metabolites, derivatives, and pharmaceutically acceptable salts of compounds of the invention. Metabolites include compounds produced by a process comprising contacting a compound of the invention with a mammal for a period of time sufficient to yield a metabolic product thereof. If the compound of the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. If the compound of the invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum, and lithium.

A compound of the invention can be in the form of a “prodrug,” which includes compounds with moieties which can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Prodrugs which are converted to active forms through other mechanisms in vivo are also included. In aspects, the compounds of the invention are prodrugs of any of the formulae herein.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Alternatively, pharmaceutically acceptable compositions of this invention may be administered in the form of suppositories for rectal or vaginal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

The compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

The amount of compounds of the present invention that may be combined with the carrier materials to produce a composition in a single dosage form will vary depending upon the host treated, the particular mode of administration. Preferably, provided compositions should be formulated so that a dosage of between 0.01 and 100 mg/kg, 0.01 and 50 mg/kg, or 1 and 25 mg/kg, body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment.

### 4. Uses of Compounds and Pharmaceutically Acceptable Compositions

Compounds and compositions described herein are generally useful for the inhibition of kinase activity of one or more enzymes. In some embodiments the kinase inhibited by the compounds and methods of the invention is SIK2

The presently disclosed compounds find use in inhibiting SIK2 protein kinase. In one embodiment, the subject matter disclosed herein is directed to a method of inhibiting SIK2, the method comprising contacting SIK2 with an effective amount of a compound of the invention or a pharmaceutical composition described herein.

The presently disclosed compounds can be used in a method for inhibiting SIK2. Such methods comprise contacting SIK2 with an effective amount of a presently disclosed compound. By “contact” is intended bringing the compound within close enough proximity to an isolated SIK2 kinase or a cell expressing SIK2 such that the compound is able to bind to and inhibit the SIK2. The compound can be contacted with SIK2 in vitro or in vivo via administration of the compound to a subject.

In one aspect, provided herein is a method of inhibiting SIK2 in a biological sample. The method comprises contacting the sample with a compound disclosed herein (such as a compound of formula I), a pharmaceutically acceptable salt thereof, or a pharmaceutical composition disclosed herein (such as a composition comprising a compound disclosed herein [such as a compound of formula I] and a pharmaceutically acceptable carrier, adjuvant, or vehicle). The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Any method known in the art to measure the kinase activity of SIK2 may be used to determine if SIK2 has been inhibited, including in vitro assays, immunoblots with antibodies specific for phosphorylated targets of SIK2, or the measurement of a downstream biological effect of SIK2 kinase activity.

Provided herein are compounds and pharmaceutical compositions that inhibit SIK2 protein kinase, as well as methods of treatment using such compounds and pharmaceutical compositions. The compounds and compositions can be used in methods of modulating the immune system, for treatment of diseases, and for treatment of cells in vivo, in vitro, or ex vivo.

The present disclosure provides methods of inhibiting SIK2 in a patient. The method comprises administering to a patient a compound disclosed herein (such as a compound of formula I), a pharmaceutically acceptable salt thereof, or a pharmaceutical composition disclosed herein (such as a composition comprising a compound disclosed herein [such as a compound of formula I] and a pharmaceutically acceptable carrier, adjuvant, or vehicle).

The presently disclosed compounds can be used to treat a SIK2-mediated disorder. As used herein, a “SIK2-mediated disorder” is a pathological condition in which SIK2 activity is necessary for the genesis or maintenance of the pathological condition.

Accordingly, in one aspect, provided herein is a method of treating a SIK2-mediated disorder, disease, or condition in a patient. The method comprises administering to said patient a compound disclosed herein (such as a compound of formula I), a pharmaceutically acceptable salt thereof, or a pharmaceutical composition disclosed herein (such as a composition comprising a compound disclosed herein [such as a compound of formula I] and a pharmaceutically acceptable carrier, adjuvant, or vehicle). In some embodiments, the present invention provided a compound disclosed herein (such as a compound of formula I), a pharmaceutically acceptable salt thereof, or a pharmaceutical composition disclosed herein for use in medicine.

In some embodiments, the SIK2-mediated disorder is selected from inflammatory diseases, autoinflammatory diseases, autoimmune diseases, proliferative diseases, fibrotic diseases, transplantation rejection, diseases involving impairment of cartilage turnover, congenital cartilage malformation, diseases involving impairment of bone turnover, diseases associated with hypersecretion of TNFα, interferons, IL-6, IL-12 and/or IL-23, respiratory diseases, endocrine and/or metabolic diseases, cardiovascular diseases, dermatological diseases, and abnormal angiogenesis associated diseases.

In some embodiments, the present invention provides a method of treating an inflammatory disease in a patient in need thereof, wherein the method comprises administering to the patient a compound disclosed herein (such as a compound of formula I) or a pharmaceutically acceptable salt thereof.

In some embodiments, the present invention provides a method of treating an autoinflammatory disease in a patient in need thereof, wherein the method comprises administering to the patient a compound disclosed herein (such as a compound of formula I) or a pharmaceutically acceptable salt thereof.

In some embodiments, the present invention provides a method of treating an autoimmune disease in a patient in need thereof, wherein the method comprises administering to the patient a compound disclosed herein (such as a compound of formula I) or a pharmaceutically acceptable salt thereof.

In some embodiments, the present invention provides a method of treating cancer in a patient in need thereof, wherein the method comprises administering to the patient a compound disclosed herein (such as a compound of formula I) or a pharmaceutically acceptable salt thereof. In some embodiments, the cancer is selected from solid tumors (e.g., bone cancer, skin cancer, prostate cancer, colon cancer, esophageal cancer, endometrial cancer, ovarian cancer, uterine cancer, renal cancer, kidney cancer, bladder cancer, hepatic cancer, pancreatic cancer, gastric cancer, breast cancer, lung cancer, cancers of the head and neck, thyroid cancer, glioblastoma, sarcoma, bile duct cancer, bladder cancer), hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), DLBCL, mantle cell lymphoma, Non-Hodgkin lymphoma (including relapsed or refractory NHL and recurrent follicular), Hodgkin lymphoma or multiple myeloma) and combinations of said cancers.

In some embodiments, the SIK2-mediated disorder is a cancer selected from ovarian cancer, breast cancer, acute myeloid leukemia, and multiple myeloma.

In some embodiments, the disease or disorder is cancer. In some embodiments, the cancer is a solid tumor cancer. In some embodiments, the cancer is lung cancer, non-small cell lung cancer (NSCLC), bone cancer, pancreatic cancer, skin cancer, cancer of the head and neck, cutaneous or intraocular melanoma, uterine cancer, ovarian cancer, colorectal cancer, anal cancer, stomach cancer, colon cancer, breast cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's Disease, hepatocellular cancer, cancer of the esophagus, cancer of the small intestine, cancer of the endocrine system (e.g., cancer of the thyroid, pancreas, parathyroid or adrenal glands), sarcomas of soft tissues, cancer of the urethra, cancer of the penis, prostate cancer, hormone-refractory prostate cancer, bladder cancer, kidney cancer, renal cell carcinoma, carcinoma of the renal pelvis, pediatric malignancy, neoplasms of the central nervous system, primary CNS lymphoma, spinal axis tumors, medulloblastoma, brain stem gliomas, or pituitary adenomas. In some embodiments, the cancer is a liquid tumor cancer. In some embodiments, the cancer is a leukemia. In some embodiments, the leukemia is acute lymphocytic leukemia (ALL), acute myelogenous leukemia (AML), chronic lymphocytic leukemia (CLL), or chronic myelogenous leukemia (CML). In some embodiments, the cancer is a lymphoma. In some embodiments, the lymphoma is small lymphocytic lymphoma (SLL), cutaneous B-cell lymphoma, cutaneous T-cell lymphoma, Hodgkin's lymphoma, non-Hodgkin's lymphoma, or Waldenstrom macroglobulinemia.

In yet another embodiment, provided herein are methods for treating diseases or disorders selected from the group consisting of inflammatory diseases, autoinflammatory diseases, autoimmune diseases, proliferative diseases, fibrotic diseases, transplantation rejection, diseases involving impairment of cartilage turnover, congenital cartilage malformation, diseases involving impairment of bone turnover, diseases associated with hypersecretion of IL-6, diseases associated with hypersecretion of TNFα, interferons, IL-12 and/or IL-23, respiratory diseases, endocrine and/or metabolic diseases, cardiovascular diseases, dermatological diseases, and abnormal angiogenesis associated diseases, in a subject in need thereof, the method comprising administering a therapeutically effective amount of a compound described herein, or a pharmaceutically acceptable salt, or stereoisomer thereof, or a pharmaceutical composition described herein.

In some embodiments, the disease or disorder is an inflammatory disease. In some embodiments, the inflammatory disease is rheumatoid arthritis, osteoarthritis, allergic airway disease (e.g., asthma), chronic obstructive pulmonary disease (COPD), or inflammatory bowel diseases (e.g., Crohn's disease, ulcerative colitis).

In some embodiments, the disease or disorder is an autoinflammatory disease. In some embodiments, the autoinflammatory disease is Cryopyrin-Associated Periodic Syndromes (CAPS), Familial Mediterranean Fever (FMF), tumor necrosis factor receptor-associated periodic syndrome (TRAPS), Behcet's disease, Systemic-onset Juvenile Idiopathic Arthritis (SJIA), Still's disease, hemophagocytic lymphohistiocytosis, or macrophage activation syndrome.

In other embodiments, the present invention also provides methods for improving, restoring or enhancing fertility and for increasing oocyte production in a female subject by administering to the female subject an effective amount of a compound described herein, or a pharmaceutically acceptable salt, or stereoisomer thereof, or a pharmaceutical composition described herein.

In some embodiments, the disease or disorder is an autoimmune disease. In some embodiments, the autoimmune disease is COPD, asthma, bronchitis, systemic lupus erythematosus (SLE), cutaneous lupus erythematosus (CLE), lupus nephritis, dermatomyositis, autoimmune hepatitis, primary sclerosing cholangitis, primary biliary cirrhosis, Sjogren's syndrome, multiple sclerosis, psoriasis, dry eye disease, type I diabetes mellitus, atopic dermatitis, thyroiditis, contact dermatitis, eczematous dermatitis, inflammatory bowel disease (e.g., Crohn's disease and ulcerative colitis), atherosclerosis, or amyotrophic lateral sclerosis.

In some embodiments, the disease or disorder is a proliferative disease. In some embodiments, the proliferative disease is cancer, myeloproliferative disorders, leukemia, multiple myeloma, psoriasis, restenosis, scleroderma, or fibrosis.

In some embodiments, the disease or disorder is a fibrotic disease. In some embodiments, the fibrotic disease is idiopathic pulmonary fibrosis (IPF), Dupuytren's disease, nonalcoholic steatohepatitis (NASH), systemic sclerosis, renal fibrosis, cutaneous fibrosis or fibrostenotic Crohn's disease.

In some embodiments, the disease or disorder involves impairment of bone turnover. In some embodiments, the term diseases involving impairment of bone turnover refers to osteoporosis, osteopenia, hormone deficiency, hormone excess, Paget's disease, osteoarthritis, renal bone disease, osteogenesis imperfecta, or hypophosphatasia.

In some embodiments, the disease or disorder is associated with hypersecretion of IL-6. In some embodiments, the disease associated with hypersecretion of IL-6 is Castleman's disease, multiple myeloma, psoriasis, Kaposi's sarcoma, or mesangial proliferative glomerulonephritis

In some embodiments, the disease or disorder is associated with hypersecretion of TNFα, interferons, IL-12 and/or IL-23. In some embodiments, the disease associated with hypersecretion of TNFα, interferons, IL-12 and/or IL-23 is systemic and cutaneous lupus erythematosus, lupus nephritis, dermatomyositis, Sjogren's syndrome, psoriasis, rheumatoid arthritis, polymyalgia rheumatica, giant cell arteritis psoriatic arthritis, multiple sclerosis, trisomy 21, ulcerative colitis, or Crohn's disease.

In some embodiments, the disease or disorder is an inflammatory disease, an autoinflammatory disease, an autoimmune disease, a proliferative disease, a fibrotic disease, transplantation rejection, a disease involving impairment of cartilage turnover, congenital cartilage malformation, a diseases involving impairment of bone turnover, a disease associated with hypersecretion of IL-6, a disease associated with hypersecretion of TNFα, interferons, IL-12 and/or IL-23, a respiratory disease, an endocrine and/or metabolic disease, a cardiovascular disease, a dermatological disease, or an abnormal angiogenesis associated disease.

In other aspects, the present invention also provides methods for treating conditions or disorders including:
Arthritis, including rheumatoid arthritis, juvenile arthritis, and psoriatic arthritis;
Autoimmune or inflammatory diseases or disorders, including Hashimoto's thyroiditis, autoimmune hemolytic anemia, autoimmune atrophic gastritis of pernicious anemia, autoimmune encephalomyelitis, autoimmune orchitis, Goodpasture's disease, autoimmune thrombocytopenia, sympathetic ophthalmia, myasthenia gravis, Graves' disease, primary biliary cirrhosis, autoimmune hepatitis, primary sclerosing cholangitis, chronic aggressive hepatitis, non-alcoholic fatty liver disease, non-alcoholic steatohepatitis ulcerative colitis and membranous glomerulopathy, systemic lupus erythematosus, rheumatoid arthritis, psoriatic arthritis, Sjogren's syndrome, Reiter's syndrome, polymyositis, dermatomyositis, type I interferonopathies including Aicardi-Goutieres syndrome and other mendelian diseases of overexpression of type I interferon systemic sclerosis, polyarteritis nodosa, multiple sclerosis, relapsing remitting multiple sclerosis, primary progressive multiple sclerosis, secondary progressive multiple sclerosis, and bullous pemphigoid, and additional autoimmune diseases, which can be O-cell (humoral) based or T-cell based, including Cogan's syndrome, ankylosing spondylitis, Wegener's granulomatosis, autoimmune alopecia, Type I or juvenile onset diabetes, gout, thyroiditis or bacterial sepsis; Diabetes, including Type I diabetes or complications from diabetes;
Eye diseases, disorders or conditions including autoimmune diseases of the eye, keratoconjunctivitis, vernal conjunctivitis, uveitis including non-infectious uveitis associated with Behcet's disease, non-infectious uveitis, and lens-induced uveitis, keratitis, herpetic keratitis, conical keratitis, corneal epithelial dystrophy, keratoleukoma, ocular premphigus, Mooren's ulcer, scleritis, Grave's ophthalmopathy, Vogt-Koyanagi-Harada syndrome, keratoconjunctivitis sicca (dry eye), phlyctenule, iridocyclitis, sarcoidosis, endocrine ophthalmopathy, sympathetic ophthalmitis, allergic conjunctivitis, or ocular neovascularization;
Intestinal inflammations, including Crohn's disease, ulcerative colitis, pouchitis, inflammatory bowel disease, celiac disease, proctitis, eosinophilic gastroenteritis, mastocytosis, gastrointestinal mucositis, oral mucositis or pouchitis;
Neurodegenerative diseases including motor neuron disease, Alzheimer's disease, Parkinson's disease, amyotrophic lateral sclerosis, Huntington's disease, cerebral ischemia, or neurodegenerative disease caused by traumatic injury, strike, glutamate neurotoxicity or hypoxia; ischemic/reperfusion injury in stroke, myocardial ischemia, renal ischemia, heart attacks, cardiac hypertrophy, atherosclerosis and arteriosclerosis, organ hypoxia, or platelet aggregation; Skin diseases, conditions or disorders including atopic dermatitis, hand dermatitis, contact dermatitis, allergic contact dermatitis, irritant contact dermatitis, neurodermatitis, perioral dermatitis, stasis dermatitis, dyshidrotic eczema, xerotic dermatitis, nummular dermatitis, seborrheic dermatitis, eyelid dermatitis, diaper dermatitis, dermatomyositis, lichen planus, lichen sclerosis, alopecia areata, vitiligo, rosacea, epidermolysis bullosa, keratosis pilaris, pityriasis alba, pemphigus, vulvovaginitis, acne, chronic spontaneous urticaria, chronic idiopathic urticaria, chronic physical urticaria, Vogt-Koyanagi-Harada disease, Sutton nevus/nevi, post inflammatory hypopigmentation, senile leukoderma, chemical/drug-induced leukoderma, cutaneous lupus erythematosus, discoid lupus, palmoplantar pustulosis, pemphigoid, sweet's syndrome, hidradenitis suppurativa, psoriasis, plaque psoriasis, pustular psoriasis, nail psoriasis, flexural psoriasis, guttate psoriasis, psoriatic arthritis, erythrodermic psoriasis, or inverse psoriasis;
Allergic reactions including allergic dermatitis in mammal (including horse allergic diseases such as bite hypersensitivity), summer eczema, sweet itch in horses, heaves, inflammatory airway disease, recurrent airway obstruction, airway hyper-responsiveness, or chronic obstruction pulmonary disease;
Asthma and other obstructive airways diseases, including chronic or inveterate asthma, late asthma, bronchitis, bronchial asthma, allergic asthma, intrinsic asthma, extrinsic asthma, or dust asthma; and
Transplant rejection, including pancreas islet transplant rejection, bone marrow transplant rejection, graft-versus-host disease, organ and cell transplant rejection such as bone marrow, cartilage, cornea, heart, intervertebral disc, islet, kidney, limb, liver, lung, muscle, myoblast, nerve, pancreas, skin, small intestine, or trachea, or xeno transplantation.

In some embodiments, the present invention provides a method of treating ovarian cancer in a patient in need thereof, wherein the method comprises administering to the patient a compound disclosed herein (such as a compound of formula I) or a pharmaceutically acceptable salt thereof.

In some embodiments, the present invention provides a method of treating breast cancer in a patient in need thereof, wherein the method comprises administering to the patient a compound disclosed herein (such as a compound of formula I) or a pharmaceutically acceptable salt thereof.

In some embodiments, the present invention provides a method of treating acute myeloid leukemia in a patient in need thereof, wherein the method comprises administering to the patient a compound disclosed herein (such as a compound of formula I) or a pharmaceutically acceptable salt thereof.

In some embodiments, the present invention provides a method of treating multiple myeloma in a patient in need thereof, wherein the method comprises administering to the patient a compound disclosed herein (such as a compound of formula I) or a pharmaceutically acceptable salt thereof.

In some embodiments, the present invention provides a method of treating fibrotic diseases in a patient in need thereof, wherein the method comprises administering to the patient a compound disclosed herein (such as a compound of formula I) or a pharmaceutically acceptable salt thereof.

In some embodiments, the present invention provides a method of treating transplantation rejection diseases involving impairment of cartilage turnover in a patient in need thereof, wherein the method comprises administering to the patient a compound disclosed herein (such as a compound of formula I) or a pharmaceutically acceptable salt thereof.

In some embodiments, the present invention provides a method of treating congenital cartilage malformation in a patient in need thereof, wherein the method comprises administering to the patient a compound disclosed herein (such as a compound of formula I) or a pharmaceutically acceptable salt thereof.

In some embodiments, the present invention provides a method of treating diseases involving impairment of bone turnover in a patient in need thereof, wherein the method comprises administering to the patient a compound disclosed herein (such as a compound of formula I) or a pharmaceutically acceptable salt thereof.

In some embodiments, the present invention provides a method of treating diseases associated with hypersecretion of TNFα, interferons, IL-6, IL-12 and/or IL-23 in a patient in need thereof, wherein the method comprises administering to the patient a compound disclosed herein (such as a compound of formula I) or a pharmaceutically acceptable salt thereof.

In some embodiments, the present invention provides a method of treating respiratory diseases in a patient in need thereof, wherein the method comprises administering to the patient a compound disclosed herein (such as a compound of formula I) or a pharmaceutically acceptable salt thereof.

In some embodiments, the present invention provides a method of treating endocrine and/or metabolic diseases in a patient in need thereof, wherein the method comprises administering to the patient a compound disclosed herein (such as a compound of formula I) or a pharmaceutically acceptable salt thereof.

In some embodiments, the present invention provides a method of treating cardiovascular diseases, including pulmonary arterial hypertension or complications from pulmonary arterial hypertension in a patient in need thereof, wherein the method comprises administering to the patient a compound disclosed herein (such as a compound of formula I) or a pharmaceutically acceptable salt thereof.

In some embodiments, the present invention provides a method of treating dermatological diseases in a patient in need thereof, wherein the method comprises administering to the patient a compound disclosed herein (such as a compound of formula I) or a pharmaceutically acceptable salt thereof.

In some embodiments, the present invention provides a method of treating abnormal angiogenesis associated diseases in a patient in need thereof, wherein the method comprises administering to the patient a compound disclosed herein (such as a compound of formula I) or a pharmaceutically acceptable salt thereof.

In some embodiments, the SIK2-mediated disorder is inflammatory bowel diseases such as Crohn's disease and ulcerative colitis, diabetes, a skin pigmentation disease, osteoporosis, osteoarthritis, lupus (systemic lupus erythematosus), a musculoskeletal disease, or rheumatoid arthritis.

In some embodiments, the SIK2-mediated disorder is Bone-mineral disorders, including chronic kidney disease. In some embodiments, the SIK2-mediated disorder is a mineral bone disorder. In some embodiments, the SIK2-mediated disorder is a mineral loss disorder, such as Malignancy-related and oncology drug-related bone loss, and osteoporosis (post-menopausal, glucocorticoid-induced, hypogonadal).

In some embodiments, the SIK2-mediated disorder is Ankylosing Spondylitis, Psoriatic Arthritis, Crohn's Disease (Active and Maintenance), Plaque Psoriasis, Juvenile Idiopathic Arthritis, Ulcerative Colitis, Hidradenitis Suppurativa, Uveitis, and non-radiographic axial spondyloarthritis.

In some embodiments, the present invention provides a method of treating inflammatory bowel disease in a patient in need thereof, wherein the method comprises administering to the patient a compound disclosed herein (such as a compound of formula I) or a pharmaceutically acceptable salt thereof.

In some embodiments, the present invention provides a method of treating diabetes in a patient in need thereof, wherein the method comprises administering to the patient a compound disclosed herein (such as a compound of formula I) or a pharmaceutically acceptable salt thereof.

In some embodiments, the present invention provides a method of treating a skin pigmentation disease in a patient in need thereof, wherein the method comprises administering to the patient a compound disclosed herein (such as a compound of formula I) or a pharmaceutically acceptable salt thereof.

In some embodiments, the present invention provides a method of treating osteoporosis in a patient in need thereof, wherein the method comprises administering to the patient a compound disclosed herein (such as a compound of formula I) or a pharmaceutically acceptable salt thereof.

In some embodiments, the present invention provides a method of treating osteoarthritis in a patient in need thereof, wherein the method comprises administering to the patient a compound disclosed herein (such as a compound of formula I) or a pharmaceutically acceptable salt thereof.

In some embodiments, the present invention provides a method of treating a musculoskeletal disease in a patient in need thereof, wherein the method comprises administering to the patient a compound disclosed herein (such as a compound of formula I) or a pharmaceutically acceptable salt thereof.

In some embodiments, the present invention provides a method of treating rheumatoid arthritis in a patient in need thereof, wherein the method comprises administering to the patient a compound disclosed herein (such as a compound of formula I) or a pharmaceutically acceptable salt thereof.

It is understood that appropriate doses of the active compound depends upon a number of factors within the knowledge of the ordinarily skilled physician or veterinarian. The dose(s) of the active compound will vary, for example, depending upon the age, body weight, general health, gender, and diet of the subject, the time of administration, the route of administration, the rate of excretion, and any drug combination.

It will also be appreciated that the effective dosage of a compound of the invention or a pharmaceutically acceptable salt, prodrug, metabolite, or derivative thereof used for treatment may increase or decrease over the course of a particular treatment. Changes in dosage may result and become apparent from the results of diagnostic assays.

In some embodiments, the SIK2 inhibitor is administered to the subject at a dose of between about 0.001 μg/kg and about 1000 mg/kg, including but not limited to about 0.001 μg/kg, 0.01 μg/kg, 0.05 μg/kg, 0.1 μg/kg, 0.5 μg/kg, 1 μg/kg, 10 μg/kg, 25 μg/kg, 50 μg/kg, 100 μg/kg, 250 μg/kg, 500 μg/kg, 1 mg/kg, 5 mg/kg, 10 mg/kg, 25 mg/kg, 50 mg/kg, 100 mg/kg, and 200 mg/kg.

In some embodiments, the SIK2 inhibitor is administered to the subject at a dose of between about 0.1 mg and about 10 g, including but not limited to about 1 mg, 5 mg, 10 mg, 25 mg, 50 mg, 100 mg, 200 mg, 250 mg, 500 mg, 750 mg, 1 g, 2 g, 5 g, and 10 g.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

Additionally, provided are SIK2 inhibitors for use as therapeutic active substances. A SIK2 inhibitor for use in treating or preventing a SIK2-mediated disorder provided herein. Further provided is the use of a SIK2 inhibitor in the manufacture of a medicament for treating or preventing a SIK2-mediated disorder provided herein.

The term “administration” or “administering” includes routes of introducing the compound(s) to a subject to perform their intended function. Examples of routes of administration which can be used include injection (subcutaneous, intravenous, parenterally, intraperitoneally, intrathecal), topical, oral, inhalation, rectal and transdermal.

The term “effective amount” includes an amount effective, at dosages and for periods of time necessary, to achieve the desired result. An effective amount of compound may vary according to factors such as the disease state, age, and weight of the subject, and the ability of the compound to elicit a desired response in the subject. Dosage regimens may be adjusted to provide the optimum therapeutic response.

The phrases “systemic administration,” “administered systemically”, “peripheral administration” and “administered peripherally” as used herein mean the administration of a compound(s), drug or other material, such that it enters the patient's system and, thus, is subject to metabolism and other like processes.

The phrase “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “subject” refers to animals such as mammals, including, but not limited to, primates (e.g., humans), cows, sheep, goats, horses, dogs, cats, rabbits, rats, mice and the like. In certain embodiments, the subject is a human.

### 5. Combination Therapies

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated.” In certain embodiments, a provided combination, or composition thereof, is administered in combination with another therapeutic agent.

Those additional agents may be administered separately from a provided combination therapy, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another.

As used herein, the term “combination,” “combined,” and related terms refers to the simultaneous or sequential administration of therapeutic agents in accordance with this invention. For example, a combination of the present invention may be administered with another therapeutic agent simultaneously or sequentially in separate unit dosage forms or together in a single unit dosage form.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

In one embodiment, the present invention provides a composition comprising a compound of formula I and one or more additional therapeutic agents. The therapeutic agent may be administered together with a compound of formula I or may be administered prior to or following administration of a compound of formula I. In certain embodiments, a compound of formula I may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a compound of formula I may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

A compound of the current invention may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can besides or in addition be administered especially for tumor therapy in combination with chemotherapy, radiotherapy, immunotherapy, phototherapy, surgical intervention, or a combination thereof. Long-term therapy is equally possible as is adjuvant therapy in the context of other treatment strategies, as described above. Other possible treatments are therapy to maintain the patient's status after tumor regression, or even chemopreventive therapy, for example in patients at risk.

### General Synthesis Description

Examples of the invention of general formula O and P can be prepared as described in scheme 1. For that purpose, methyl pyridine derivative A is converted to benzyl bromide B under radical bromination conditions. Substitution of benzylic bromide in compound B with a cyanide leads to nitrile C. Compound C upon treatment with an N-transfer reagent such as O-(mesitylsulfonyl)hydroxylamine (generated in situ from tert-butyl ((mesitylsulfonyl)oxy)carbamate upon treatment with trifluoroacetic acid) is converted to 1-aminopyridinium derivative D. Compound D undergoes cyclization in the presence of base such as potassium carbonate in an organic solvent such as methanol to form heterocycle E. The amino group in E is protected by means of heating with phthalic anhydride in a solvent such as toluene to give phthalimide derivative F. Compound F is reacted with a boronic acid of general formula G (R¹=C₁-C₃-alkyl, optionally fluorinated) under Suzuki-Miyaura cross-coupling conditions to yield cross-coupling product H. Preparation of boronic acids of general formular G is described in the experimental section of the description of the present invention. Treatment of compound H with a Lewis acid such as aluminum (III) chloride in aprotic organic solvent such as 1,2-dichloroethane leads to intermediate I. Alkylation of I with a bifunctional alkylation reagent of general formula J (X=halogen, OTs, OTf, OMs; n=1 or 2; and R² is as defined for R^{3a} and R^{3b} in the description of the invention) in the presence of base such as potassium carbonate in a polar aprotic solvent such as N,N-dimethylacetamide leads to the formation of polycyclic compound K. Treatment of K with hydrazine hydrate in a polar protic solvent such as ethanol leads to heteroaryl amine L. Intermediate L is further used in the acylation reactions using suitably functionalized acylation or sulfonylation reagents of general formula M or N (where Y is for example O-alkyl, OH, halogen; and L₆ is as defined in the description of the present invention) to provide examples of the invention O or P. Preparation of acylation reagents or sulfonylation reagents of general formula M and N as well as use of such reagents in reactions with heteroaromatic amines is well known to the person skilled in the art of organic synthesis. Respective example experimental protocols can be found in the experimental section of the description of the invention.

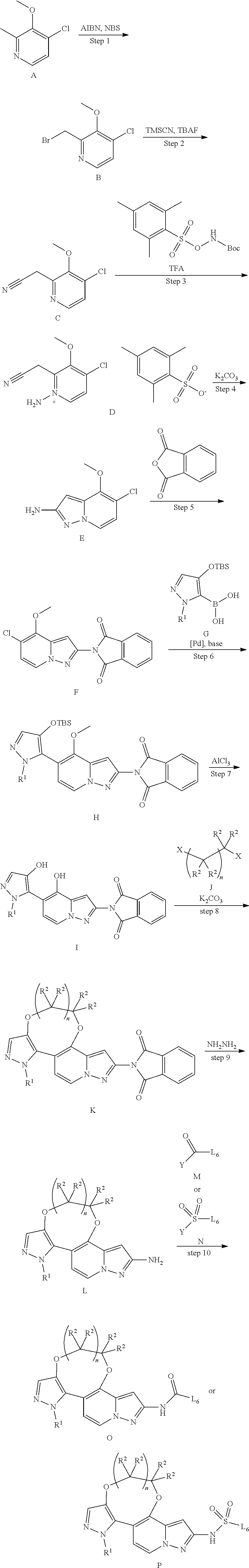

### EXAMPLES

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein. Additional compounds of the invention were prepared by methods substantially similar to those described herein in the Examples and methods known to one skilled in the art.

### List of Abbreviations

AlCl₃: aluminum chloride
CCl₄: tetrachloromethane
NBS: N-bromosuccinimide
AIBN: 2,2′-azobis(2-methylpropionitrile)
TMSCN: trimethylsilylformonitrile
TFA: trifluoroacetic acid
FA: formic acid
TEA: triethylamine
THF: tetrahydrofuran
DCM: dichloromethane
DCE: 1,2-dichloroethane
DMA: N,N-dimethylacetamide
DMF: N, N-dimethylformamide
DEAD: diethyl azodicarboxylate
PE: petroleum ether
EtOAc: ethyl acetate
TBAF: tetra-n-butylammonium fluoride
CsF: cesium fluoride
MeOH: methanol
EtOH: ethanol
NMP: N-methyl-2-pyrrolidone
n-BuLi: n-Butyllithium
t-BuOK: potassium tert-butoxide
LiHMDS: lithium hexamethyldisilazide
LDA: lithium diisopropylamide
EtSNa: sodium ethanethiolate
XPhos Pd G4: methanesulfonato(2-dicyclohexylphosphino-2′,4′,6′-tri-i-propyl-1,1′-biphenyl) (2′-methylamino-1,1′-biphenyl-2-yl) palladium(II)
XantPhos: [5-(diphenylphosphanyl)-9,9-dimethyl-9H-xanthen-4-yl]diphenylphosphane
t-BuBrettPhos-Pd-G3: [(2-di-tert-butylphosphino-3,6-dimethoxy-2′,4′,6′-triisopropyl-1,1′-biphenyl)-2-(2′-amino-1,1′-biphenyl)]palladium(II) methanesulfonate
Pd(OAc)₂: palladium acetate
PPh₃: triphenylphosphine
ACN: acetonitrile
NH₄Cl: ammonium chloride
NH₂NH₂·H₂O: hydrazine hydrate
TsOH·H₂O: 4-methylbenzenesulfonic acid hydrate
N₂: nitrogen
H₂O: water
H₂O₂: hydrogen peroxide
K₃PO₄: potassium phosphate
K₂CO₃: potassium carbonate
Cs₂CO₃: cesium carbonate
Na₂SO₄: sodium sulfate
Na₂S₂O₃: sodium thiosulfate
NaHCO₃: sodium bicarbonate
TBS: tert-butyldimethylsilyl
TBSCl: tert-butyldimethylchlorosilane
CDCl₃: deuterated chloroform
DMSO-d₆: deuterated dimethyl sulfoxide
CD₃OD: deuterated methanol
cataCXium A: di(1-adamantyl)-n-butylphosphine
iPrMgCl: isopropylmagnesium chloride
iPrMgCl·LiCl: isopropylmagnesium chloride-lithium chloride complex
HATU: 2-(7-azabenzotriazol-1-yl)-N,N,N′,N′-tetramethyluronium hexafluorophosphate
DAST: N,N-diethyl-S,S,S-trifluoro-λ⁴-sulfanamine
IPA: isopropanol
NaOAc: Sodium acetate
AlMe₃: trimethylaluminum
TosCl: para-toluensulfonyl chloride
DMAP: 4-dimethylaminopyridine
cataCXium® A: di(1-adamantyl)-n-butylphosphine
cataCXium® A Pd G3: [(di(1-adamantyl)-butylphosphine)-2-(2′-amino-1,1′-biphenyl)]-palladium(II) methanesulfonate
sat.: saturated
aq.: aqueous
Ac₂O: acetic anhydride
BnBr: benzyl bromide
m-CPBA: 3-chloroperoxybenzoic acid
DBU: 1,8-diazabicyclo[5.4.0]undec-7-ene
DIC: N,N′-diisopropyl carbodiimide
DIAD: diisopropyl azodicarboxylate
K₂OsO₄·2H₂O: potassium osmate(VI) dihydrate
MTBE: methyl tert-butyl ether
MOMBr: bromo(methoxy)methane
MeMgBr: methyl magnesium bromide
NaIO₄: sodium periodate
NaSMe: sodium thiomethoxide
NiCl₂(dme): dichloro(dimethoxyethane)nickel
T4P: 1,3,5,2,4,6-trioxatriphosphorinane, 2,4,6-tributyl-, 2,4,6-trioxide
TfOH: trifluoromethanesulfonic acid
TBAL: tetrabutylammonium iodide
t-BuXPhos-Pd-G3: Methanesulfonato(2-di-t-butylphosphino-2′,4′,6′-tri-i-propyl-1,1′-biphenyl)
(2′-amino-1,1′-biphenyl-2-yl)palladium(II)
Pd(dppf)Cl₂*CH₂Cl₂: 1,1′-Bis(diphenylphosphino)ferrocene-palladium (II)dichloride
dichloromethane complex
Pd(P(t-Bu)₃)₂: bis(tri-tert-butylphosphine) palladium(0)

### Synthesis of Intermediates

### Intermediate-1: 5-chloro-4-methoxypyrazolo[1,5-a]pyridin-2-amine

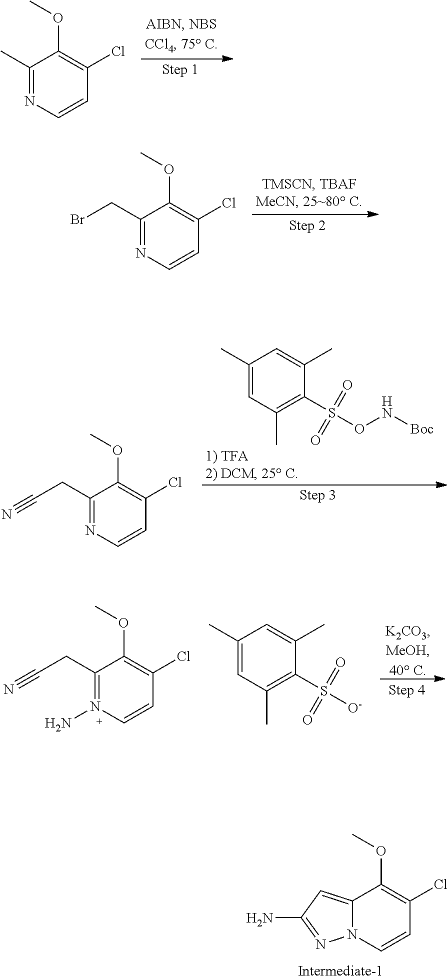

Step 1. Synthesis of 2-(bromomethyl)-4-chloro-3-methoxypyridine. To the solution of 4-chloro-3-methoxy-2-methyl-pyridine (50.00 g, 317.26 mmol, 1.00 eq) in CCl₄ (500 mL) was added the NBS (84.70 g, 475.90 mmol, 1.50 eq) and AIBN (5.21 g, 31.73 mmol, 0.10 eq) at 25° C. and the resulting mixture was stirred at 75° C. for 12 h. The reaction mixture was filtered and the filtrate was concentrated under reduced pressure. The residue was purified by flash silica gel chromatography (Eluent of PE/EtOAc) to afford the title compound. LCMS: 237.8 [M+H]⁺.

Step 2. Synthesis of 2-(4-chloro-3-methoxypyridin-2-yl)acetonitrile. To the solution of 2-(bromomethyl)-4-chloro-3-methoxypyridine (60.00 g, 253.71 mmol, 1.00 eq) in ACN (500 mL) was added the TMSCN (30.20 g, 304.45 mmol, 1.20 eq) at 25° C. and the resulting mixture was stirred at 25° C. for 0.5 hour. TBAF (1 M in THF, 381 mL, 380.56 mmol, 1.50 eq) was added at 25° C. and the resulting mixture was stirred at 80° C. for another 0.5 h. After cooling to room temperature, the reaction mixture was diluted with H₂O (800 mL) and then extracted with EtOAc (200 mL×3). The combined organic layers were dried over anhydrous Na₂SO₄, filtered and the filtrate was concentrated under reduced pressure. The residue was purified by flash silica gel chromatography (Eluent of PE/EtOAc) to afford the title compound. ¹H NMR (400 MHz, DMSO-d₆) δ=8.30 (d, 1H), 7.61 (d, 1H), 4.27 (s, 2H), 3.87 (s, 3H). LCMS: 183.0 [M+H]⁺.

Step 3. Synthesis of 1-amino-4-chloro-2-(cyanomethyl)-3-methoxypyridin-1-ium 2,4,6-trimethylbenzenesulfonate. A solution of (tert-butoxycarbonylamino)-2,4,6-trimethylbenzenesulfonate (96.72 g, 306.67 mmol, 2.80 eq) in TFA (271 g, 2.38 mol, 21.70 eq) was stirred at 0° C. for 1 hour. The reaction mixture was poured into water (200 mL) at 0° C. with vigorous stirring. The precipitate was filtered and dissolved in DCM (400 mL), then 2-(4-chloro-3-methoxypyridin-2-yl)acetonitrile (40.00 g, 219.06 mmol, 1.00 eq) was added and the resulting mixture was stirred at 20° C. for 12 h. The reaction mixture was concentrated under reduced pressure to afford the title compound which was used in the next step without further purification. LCMS: 198.2 [M]⁺.

Step 4. Synthesis of 5-chloro-4-methoxypyrazolo[1,5-a]pyridin-2-amine. To the solution of 1-amino-4-chloro-2-(cyanomethyl)-3-methoxypyridin-1-ium 2,4,6-trimethylbenzenesulfonate (20.00 g, 50.37 mmol, 1.00 eq) in MeOH (220.00 mL) was added K₂CO₃ (27.83 g, 201.38 mmol, 4.00 eq) at 25° C. and the resulting mixture was stirred at 40° C. for 12 h. The reaction mixture was diluted with H₂O (300 mL) and extracted with EtOAc:MeOH 5:1 (300 mL×3). The combined organic layers were dried over anhydrous Na₂SO₄, filtered, and the filtrate was concentrated under reduced pressure to give a residue. The residue was purified by silica gel chromatography (Eluent of PE/EtOAc) to afford the title compound. LCMS: 197.9 [M+H]⁺. ¹H NMR (400 MHz, DMSO-d₆) δ=8.09 (d, 1H), 6.53 (d, 1H), 5.75 (s, 1H), 5.44 (s, 2H), 3.90 (s, 3H).

### Intermediate-2: ethyl (1S,2R)-2-(5-methylpyridin-2-yl)cyclopropane-1-carboxylate

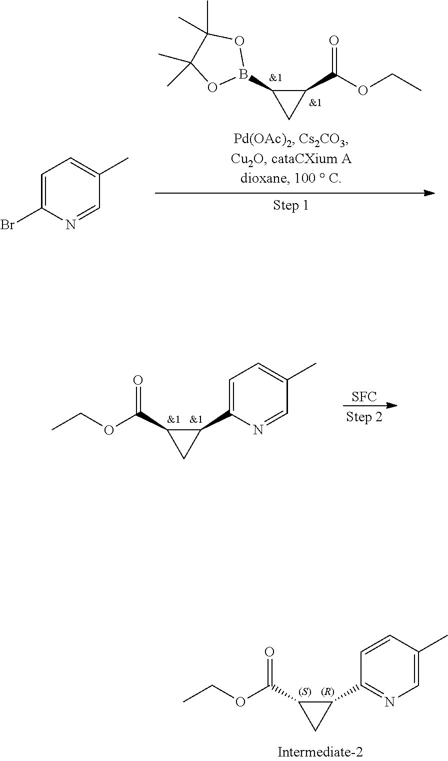

Step 1. Synthesis of rac-ethyl (1R,2S)-2-(5-methylpyridin-2-yl)cyclopropane-1-carboxylate. To the solution of 2-bromo-5-methyl-pyridine (100 g, 581.32 mmol, 1.00 eq) and rac-ethyl (1R,2S)-2-(4,4,5,5-tetramethyl-1,3,2-dioxaborolan-2-yl)cyclopropane-1-carboxylate (153.53 g, 639.45 mmol, 1.10 eq) in dioxane (1200 mL) was added Cs₂CO₃ (378.81 g, 1.16 mol, 2.00 eq), Pd(OAc)₂ (13.05 g, 58.13 mmol, 0.10 eq), bis(1-adamantyl)-butylphosphane (41.69 g, 116.26 mmol, 0.20 eq) and H₂O (60 mL) at 25° C. and the reaction mixture was degassed, purged with N₂ and then stirred at 100° C. for 12 h under N₂ atmosphere. The resulting mixture was filtered and the filtrate was concentrated under reduced pressure to give a residue. The residue was purified by silica gel chromatography (Eluent of PE/EA) to afford the title compound which was used in the next step of chiral SFC resolution.

Step 2. Synthesis of ethyl (1S,2R)-2-(5-methylpyridin-2-yl)cyclopropane-1-carboxylate. The rac-ethyl (1R,2S)-2-(5-methylpyridin-2-yl)cyclopropane-1-carboxylate (110.00 g, 535.93 mmol, 1.00 eq) was purified by chiral SFC (SFC preparative method: instrument: Waters 150Q Preparative SFC system; column: Daicel Chiralpak IG column, 250×50 mm I.D., 10 μm particle size; Mobile phase A: CO₂, Mobile Phase B: EtOH:ACN=4:1 (0.1% NH₃H₂O); Isocratic elution: 60% Phase B in Supercritical C₂; Flow rate: 120 g/min; Retention time: Peak: 4.10 min (desired), Peak2: 6.49 min; Back Pressure: 80 bar to keep the CO₂ in Supercritical flow; Wave Length: 220 nm). ¹H NMR (400 MHz, CDCl₃) δ=8.28 (s, 1H), 7.47-7.30 (m, 1H), 7.11 (d, 1H), 3.99-3.77 (m, 2H), 2.73-2.58 (m, 1H), 2.24 (s, 3H), 2.18-2.01 (m, 1H), 1.82-1.72 (m, 1H), 1.43-1.28 (m, 1H), 1.01 (t, 3H). LCMS: 206.1 [M+H]⁺.

### SFC Analytic Method:

| Instrument | SHIMADZU LC-30ADsf | | | |
|---|---|---|---|---|
| column | Chiralpak IG-3 50 × 4.6 mm I.D., particle size 3 μm | | | |
| Mobile Phase | Phase A for CO₂ | | | Phase B for EtOH (0.05% |
| | | | | diethylamine) |
| | Time(min) | A(%) | B(%) | |
| Gradient | 0.01 | 95 | 5 | |
| | 1.80 | 60 | 40 | |
| | 2.70 | 60 | 40 | |
| | 2.71 | 95 | 5 | |
| | 3.00 | 95 | 5 | |

| | | |
|---|---|---|
| Flow rate | 3.0 | mL/min |
| Column Temp | 35° | C. |
| Back pressure | 100 | bar |
| UV detection | 220 | nm |

| | |
|---|---|
| Reak retention time | 1.155 (Intermediate-2, desired stereoisomer, first eluting) |
| (min) | 1.998 (undesired stereoisomer) |

### Intermediate-3: (1 S,2R)—N-(5-chloro-4-methoxypyrazolo[1,5-a]pyridin-2-yl)-2-(5-methylpyridin-2-yl)cyclopropane-1-carboxamide

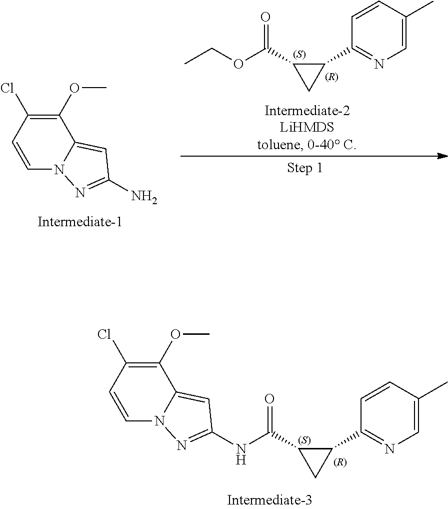

Step 1. To a solution of 5-chloro-4-methoxypyrazolo[1,5-a]pyridin-2-amine (Intermediate-1) (400 mg, 2.02 mmol, 1 eq) and ethyl (1S,2R)-2-(5-methylpyridin-2-yl)cyclopropane-1-carboxylate (Intermediate-2) (415 mg, 2.02 mmol, 1 eq) in toluene (4 mL) was added LiHMDS (1 M in THF, 4.05 mL, 2 eq) dropwise at 0° C. under N₂ atmosphere. The reaction mixture was warmed up to 40° C. and stirred for 1 h. After cooling down to room temperature, the reaction mixture was added dropwise into saturated NH₄Cl aqueous solution (30 mL) and extracted with EtOAc (50 mL×3). The combined organic layers were washed with brine (30 mL), dried over anhydrous Na₂SO₄, filtered and the filtrate was concentrated under reduced pressure to give a residue. The residue was purified by flash silica gel chromatography (Eluent of DCM/MeOH) to afford the title compound. ¹H NMR (400 MHz, CDCl₃) δ=10.23 (s, 1H), 8.41 (d, 1H), 8.03-7.96 (m, 1H), 7.48-7.42 (m, 1H), 7.28 (s, 1H), 6.98 (s, 1H), 6.60 (d, 1H), 4.00 (s, 3H), 2.70-2.58 (m, 1H), 2.30 (s, 3H), 2.28-2.21 (m, 1H), 2.04-1.97 (m, 1H), 1.66-1.57 (m, 1H). LCMS: 357.2 [M+H]⁺.

### Intermediate-4: (1S,2R)—N-(4-hydroxy-5-(4-hydroxy-1-methyl-1H-pyrazol-5-yl)pyrazolo[1,5-a]pyridin-2-yl)-2-(5-methylpyridin-2-yl)cyclopropane-1-carboxamide

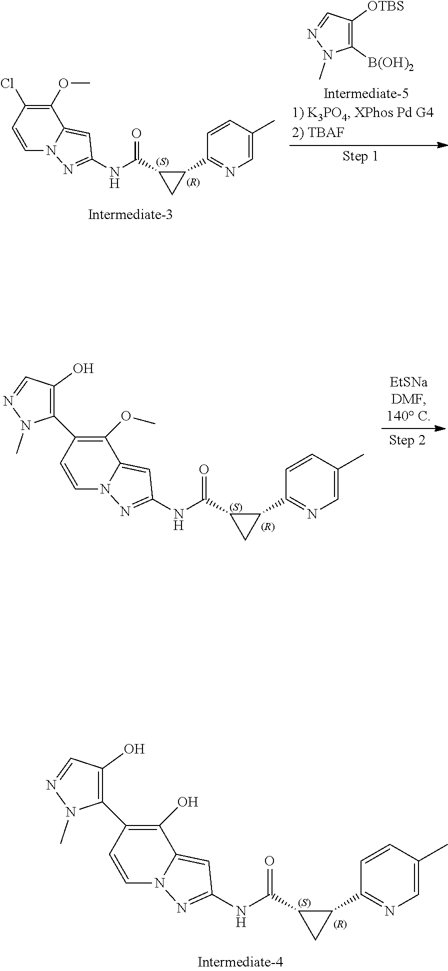

Step 1. Synthesis of (1S,2R)—N-(5-(4-hydroxy-1-methyl-1H-pyrazol-5-yl)-4-methoxypyrazolo[1,5-a]pyridin-2-yl)-2-(5-methylpyridin-2-yl)cyclopropane-1-carboxamide. To a solution of (1S,2R)—N-(5-chloro-4-methoxypyrazolo[1,5-a]pyridin-2-yl)-2-(5-methylpyridin-2-yl)cyclopropane-1-carboxamide (Intermediate-3) (500 mg, 1.40 mmol, 1 eq) and (4-((tert-butyldimethylsilyl)oxy)-1-methyl-1H-pyrazol-5-yl)boronic acid (Intermediate-5) (718 mg, 2.80 mmol, 2 eq) in 1,4-dioxane (10 mL) was added K₃PO₄ (1.5 M in H₂O, 1.87 mL, 2 eq) and XPhos Pd G4 (121 mg, 140 μmol, 0.1 eq). The resulting mixture was degassed with N₂ and then stirred at 40° C. for 1 h under N₂ atmosphere. TBAF (1 M in THF, 2.10 mL, 1.5 eq) was added and the reaction mixture was stirred at 25° C. for 1 h, then filtered and the filtrate was concentrated under reduced pressure to give a residue. The residue was purified by flash silica gel chromatography (Eluent of DCM/MeOH) followed by reverse phase preparative HPLC (MeCN/H₂O, 0.1% FA) to afford the title compound. ¹H NMR (400 MHz, DMSO-d₆) δ=11.04 (s, 1H), 8.50 (s, 1H), 8.30 (d, 1H), 8.21 (s, 1H), 7.48-7.42 (m, 1H), 7.18 (d, 1H), 7.09 (s, 1H), 6.73 (s, 1H), 6.62 (d, 1H), 3.67 (s, 3H), 3.55 (s, 3H), 2.65-2.59 (m, 1H), 2.43-2.30 (m, 1H), 2.21 (s, 3H), 1.78-1.68 (m, 1H), 1.43-1.32 (m, 1H). LCMS: 419.3 [M+H]⁺.

Step 2. Synthesis of (1S,2R)—N-(4-hydroxy-5-(4-hydroxy-1-methyl-1H-pyrazol-5-yl)pyrazolo[1,5-a]pyridin-2-yl)-2-(5-methylpyridin-2-yl)cyclopropane-1-carboxamide. A solution of (1S,2R)—N-(5-(4-hydroxy-1-methyl-1H-pyrazol-5-yl)-4-methoxypyrazolo[1,5-a]pyridin-2-yl)-2-(5-methylpyridin-2-yl)cyclopropane-1-carboxamide (80 mg, 191 μmol, 1 eq) and EtSNa (161 mg, 1.91 mmol, 10 eq) in DMF (2 mL) was stirred at 140° C. for 2 h. After cooling down to the room temperature the reaction mixture was filtered and the filtrate was concentrated under reduced pressure to give a residue. The residue was purified by flash silica gel chromatography (Eluent of DCM/MeOH) to afford the title compound. ¹H NMR (400 MHz, CD₃OD) δ=8.24 (s, 1H), 8.00 (d, 1H), 7.60-7.51 (m, 1H), 7.30 (d, 1H), 7.19-7.15 (m, 1H), 6.79 (s, 1H), 6.64 (d, 1H), 3.67 (s, 3H), 2.75-2.67 (m, 1H), 2.44-2.37 (m, 1H), 2.29 (s, 3H), 1.91-1.84 (m, 1H), 1.53-1.47 (m, 1H). LCMS: 405.2 [M+H]⁺.

### Intermediate-5: (4-((tert-butyldimethylsilyl)oxy)-1-methyl-1H-pyrazol-5-yl)boronic acid

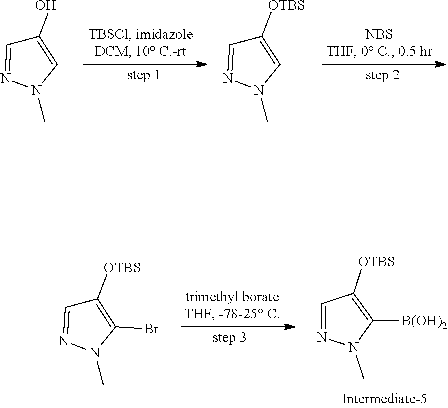

Step 1. Synthesis of 4-((tert-butyldimethylsilyl)oxy)-1-methyl-1H-pyrazole. To a solution of 1-methylpyrazol-4-ol (100.00 g, 1.02 mol, 1.00 eq) in DCM (1.00 L) was added imidazole (138.79 g, 2.04 mol, 2.00 eq), then TBSCl (169.00 g, 1.12 mol, 137.96 mL, 1.10 eq) was added at 10° C. in portions. The mixture was warmed to 25° C. and stirred for 1 h. The reaction mixture was diluted with DCM (500 mL) followed by water (800 mL). The organic layer was separated, washed with 1M aqueous HCl (700 mL×3) followed by saturated aqueous NH₄Cl (500 mL×3), dried over anhydrous Na₂SO₄ and concentrated under reduced pressure to afford the title compound which was used in the next step without further purification.

Step 2. Synthesis of 5-bromo-4-((tert-butyldimethylsilyl)oxy)-1-methyl-1H-pyrazole. To a solution of 4-((tert-butyldimethylsilyl)oxy)-1-methyl-1H-pyrazole (250.00 g, 1.18 mol, 1.00 eq) in tetrahydrofuran (2 L) was added NBS (220.00 g, 1.24 mol, 1.05 eq) at 0° C. The reaction mixture was stirred at 0° C. for 0.5 h and then quenched with saturated aqueous Na₂S₂O₃ (1.5 L) at 0° C. Then aqueous phase was separated and extracted with PE (600 mL×2). All the organic phases were combined and washed with saturated aqueous NH₄Cl (500 mL×3), dried over anhydrous Na₂SO₄, filtered and concentrated under reduced pressure to give a residue. The residue was purified by flash silica gel chromatography (Eluent of PE/EtOAc) to afford the title compound. ¹H NMR (400 MHz, CDCl₃) δ=7.14 (s, 1H), 3.80 (s, 3H), 1.00 (s, 9H), 0.19 (s, 6H). LCMS: 290.9 [M+H]⁺.

Step 3. (4-((tert-butyldimethylsilyl)oxy)-1-methyl-1H-pyrazol-5-yl)boronic acid. To a solution of 5-bromo-4-((tert-butyldimethylsilyl)oxy)-1-methyl-1H-pyrazole (50.00 g, 171.67 mmol, 1.00 eq) in anhydrous THF (1.2 L) was added n-BuLi (2.50 M in hexane, 103.00 mL, 1.50 eq) dropwise at −78° C. under N₂ atmosphere. The mixture was stirred at −78° C. for 0.5 h. Trimethyl borate (71.35 g, 686.67 mmol, 77.56 mL, 4.00 eq) was added dropwise at −78° C. The reaction mixture was allowed to warm up to 25° C. and then stirred for 1 h under N₂ atmosphere. The reaction mixture was quenched by addition of saturated aqueous NH₄Cl (500 mL) at 0° C. under N₂ atmosphere. The aqueous phase was separated and extracted with EtOAc (200 mL×3). All organic phases were combined, dried over anhydrous Na₂SO₄, filtered and concentrated under reduced pressure to give a residue. The residue was purified by flash silica gel chromatography (Eluent of PE/EtOAc) to afford the title compound. ¹H NMR (400 MHz, DMSO-d₆) δ=7.78 (s, 2H), 7.06 (s, 1H), 3.82 (s, 3H), 0.93 (s, 9H), 0.16 (s, 6H). LCMS: 257.3 [M+H]⁺.

### Intermediate-6: 2-(5-chloro-4-methoxypyrazolo[1,5-a]pyridin-2-yl)isoindoline-1,3-dione

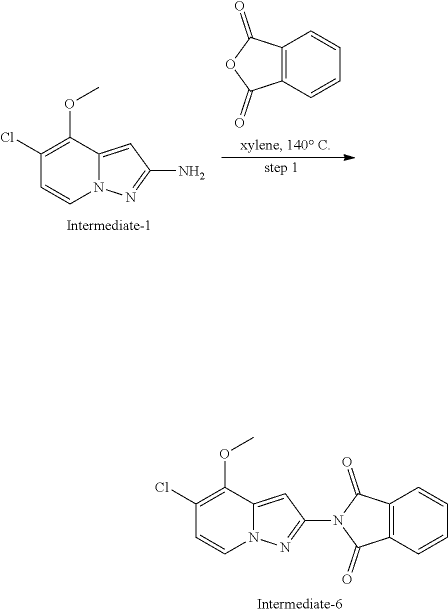 [SMILES: COc1c(Cl)ccn2nc(N)cc12 ; O=C1OC(=O)c2ccccc21 ; COc1c(Cl)ccn2nc(N3C(=O)c4ccccc4C3=O)cc12]

Step 1. To a solution of 5-chloro-4-methoxypyrazolo[1,5-a]pyridin-2-amine (Intermediate-1) (2.70 g, 13.66 mmol, 1.0 eq) in xylene (60 mL) was added isobenzofuran-1,3-dione (2.02 g, 13.66 mmol, 1.0 eq), and the reaction mixture was stirred at 140° C. for 2 h. The resulting mixture was cooled down to room temperature and MTBE (100 mL) was added. The resulting suspension was filtered. The filter cake was further triturated with MTBE (100 mL) at 20° C. for 30 min and then filtered. The filter cake was dried in vacuo to afford the title compound, which was used into the next step without further purification. LCMS: 328.0 [M+H]⁺.

### Alternative Procedure Intermediate-6: 2-(5-chloro-4-methoxypyrazolo[1,5-a]pyridin-2-yl)isoindoline-1,3-dione

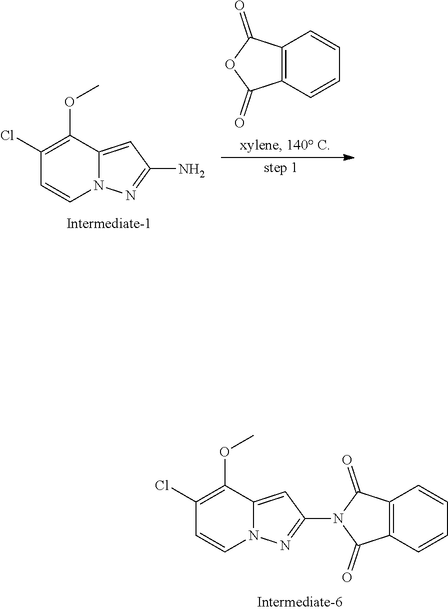 [SMILES: COc1c(Cl)ccn2nc(N)cc12 ; O=C1OC(=O)c2ccccc21 ; COc1c(Cl)ccn2nc(N3C(=O)c4ccccc4C3=O)cc12]

Step 1. Synthesis of 2-(5-chloro-4-methoxypyrazolo[1,5-a]pyridin-2-yl)isoindoline-1,3-dione.

To a solution of 5-chloro-4-methoxy-pyrazolo[1,5-a]pyridin-2-amine (Intermediate-1) (120.00 g, 607.22 mmol, 1.0 eq) in xylene (1500 mL) was added isobenzofuran-1,3-dione (116.92 g, 789.39 mmol, 1.3 eq), and it was stirred at 140° C. overnight. The reaction mixture was cooled down and left for 8 h at room temperature. The resulting mixture was filtered, the filter cake was washed with MTBE (500 mL) and then dried under reduced pressure to afford the title compound which was used in the next step without further purification. LCMS: 327.9 [M+H]⁺

### Intermediate-7: 7-methyl-2,3-dihydro-7H-pyrazolo[1,5-a]pyrazolo[3′,4′:7,8][1,4]dioxocino-[5,6-c]pyridin-12-amine

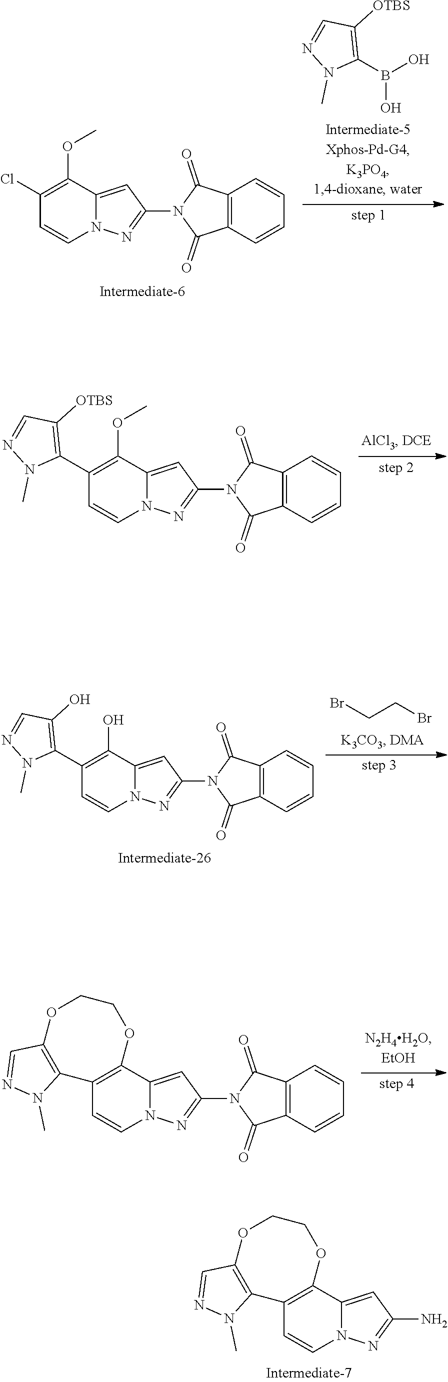

Step 1. Synthesis of 2-(5-(4-((tert-butyldimethylsilyl)oxy)-1-methyl-1H-pyrazol-5-yl)-4-methoxypyrazolo[1,5-a]pyridin-2-yl)isoindoline-1,3-dione. To a solution of 2-(5-chloro-4-methoxypyrazolo[1,5-a]pyridin-2-yl)isoindoline-1,3-dione (Intermediate-6) (3.10 g, 9.46 mmol, 1.0 eq) and (4-((tert-butyldimethylsilyl)oxy)-1-methyl-1H-pyrazol-5-yl)boronic acid (Intermediate-5) (4.85 g, 18.92 mmol, 2.0 eq) in 1,4-dioxane (30 mL) and H₂O (6 mL) was added K₃PO₄ (4.02 g, 18.92 mmol, 2.0 eq) and XPhos-Pd-G4 (814 mg, 946 μmol, 0.1 eq). The resulting mixture degassed with N₂ and it was stirred at 40° C. for 1 h under N₂ atmosphere. The reaction mixture was diluted with H₂O (15 mL) and extracted with EtOAc (25 mL×2). The combined organic layers were washed with brine (20 mL), dried over anhydrous Na₂SO₄, filtered and the filtrate was concentrated under reduced pressure. The residue was purified by silica gel chromatography (EtOAc/PE) to afford the title compound. LCMS: 504.3 [M+H]⁺.

Step 2. Synthesis of 2-(4-hydroxy-5-(4-hydroxy-1-methyl-1H-pyrazol-5-yl)pyrazolo[1,5-a]pyridin-2-yl)isoindoline-1,3-dione. To a solution of 2-(5-(4-((tert-butyldimethylsilyl)oxy)-1-methyl-1H-pyrazol-5-yl)-4-methoxypyrazolo[1,5-a]pyridin-2-yl)isoindoline-1,3-dione (3.90 g, 7.74 mmol, 1.0 eq) in DCE (70 mL) was added AlCl₃ (5.16 g, 38.72 mmol, 5.0 eq), and the resulting mixture was stirred at 80° C. for 2 h. The reaction mixture was cooled to room temperature, quenched with H₂O (1.5 mL), and the resulting mixture was purified directly by silica gel chromatography (EtOAc/PE) to afford the title compound. LCMS: 376.0 [M+H]⁺.

Step 3. Synthesis of 2-(7-methyl-2,3-dihydro-7H-pyrazolo[1,5-a]pyrazolo[3′,4′:7,8][1,4]dioxocino[5,6-c]pyridin-12-yl)isoindoline-1,3-dione. To a solution of 2-(4-hydroxy-5-(4-hydroxy-1-methyl-1H-pyrazol-5-yl)pyrazolo[1,5-a]pyridin-2-yl)isoindoline-1,3-dione (520 mg, 1.39 mmol, 1.0 eq) in DMA (10 mL) was added 1,2-dibromoethane (390 mg, 2.08 mmol, 1.5 eq) and K₂CO₃ (766 mg, 5.54 mmol, 4.0 eq), and the resulting mixture was stirred at 120° C. for 1 h. The reaction mixture was cooled to room temperature, diluted with H₂O (15 mL) and extracted with EtOAc (25 mL×2). The combined organic layers were washed with brine (20 mL), dried over anhydrous Na₂SO₄, filtered and the filtrate was concentrated under reduced pressure. The residue was purified by silica gel chromatography (EtOAc/PE) to afford the title compound. LCMS: 402.1 [M+H]⁺.

Step 4. Synthesis of 7-methyl-2,3-dihydro-7H-pyrazolo[1,5-a]pyrazolo[3′,4′:7,8][1,4]dioxocino[5,6-c]pyridin-12-amine. To a solution of 2-(7-methyl-2,3-dihydro-7H-pyrazolo[1,5-a]pyrazolo[3′,4′:7,8][1,4]dioxocino[5,6-c]pyridin-12-yl)isoindoline-1,3-dione (1.40 g, 3.49 mmol, 1.0 eq) in EtOH (7 mL) was added hydrazine hydrate (8 mL, 98 wt %), and the resulting mixture was stirred at room temperature for 1 h. The reaction mixture was diluted with H₂O (20 ml) and extracted with EtOAc (25 mL×4). The combined organic layers were washed with brine (20 mL), dried over anhydrous Na₂SO₄, filtered and the filtrate was concentrated under reduced pressure. The residue was purified by silica gel chromatography (EtOAc/PE), followed by reverse phase HPLC (C18 column, water (0.1% FA)-ACN to afford the title compound. LCMS: 272.0 [M+H]⁺.

### Intermediate-8: (1S,2R)-2-(4,4,5,5-tetramethyl-1,3,2-dioxaborolan-2-yl)cyclopropane-1-carboxylate

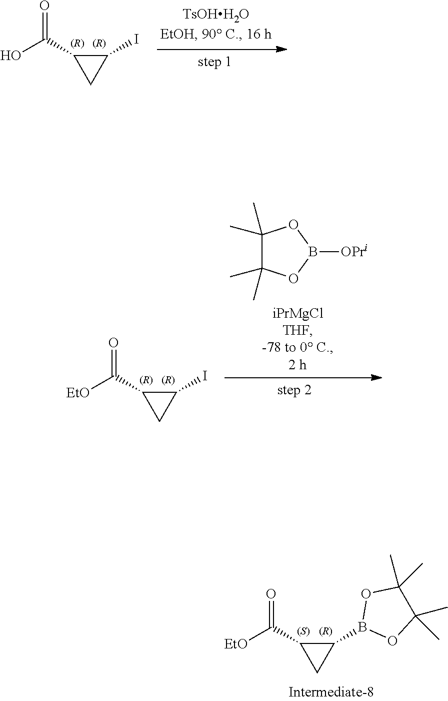

Step 1. Synthesis of ethyl (1R,2R)-2-iodocyclopropane-1-carboxylate. To a solution of (1R,2R)-2-iodocyclopropane-1-carboxylic acid (85.00 g, 400.97 mmol, 1.00 eq) in EtOH (700 mL) was added TsOH·H₂O (76.27 g, 400.97 mmol, 1.00 eq) and the reaction mixture was stirred at 90° C. for 16 h. The resulting mixture was concentrated under reduced pressure, diluted with EtOAc (500 mL), washed with water (300 mL×2), sat. aq. NaHCO₃ (300 mL×2), and brine (300 mL×3). The organic layer was dried over Na₂SO₄, filtered and the filtrate was concentrated under vacuum to afford the title compound which was used in the next step without further purification. LCMS: 240.9 [M+H]⁺. ¹H NMR: (400 MHz, CDCl₃) δ=4.29-4.16 (m, 2H), 2.85-2.75 (m, 1H), 1.90-1.82 (m, 1H), 1.55-1.47 (m, 1H), 1.43-1.36 (m, 1H), 1.33-1.27 (m, 3H).

Step 2. Synthesis of ethyl (1S,2R)-2-(4,4,5,5-tetramethyl-1,3,2-dioxaborolan-2-yl)cyclopropane-1-carboxylate. To a stirred solution of ethyl (1R,2R)-2-iodocyclopropane-1-carboxylate (106.00 g, 441.60 mmol, 1.00 eq) in THF (1.00 L) was added iPrMgCl (2.00 M, 331.20 mL, 1.50 eq) dropwise over 1 h at −78° C. under N₂ atmosphere (during addition the temperature was kept below −65° C.). The reaction mixture was stirred at −78° C. for 1 h and then 2-isopropoxy-4,4,5,5-tetramethyl-1,3,2-dioxaborolane (123.24 g, 662.39 mmol, 135.13 mL, 1.50 eq) was added. The reaction mixture was warmed to 0° C. gradually over 1 h. The resulting mixture was quenched with saturated aqueous NH₄Cl (400 mL) and extracted with EtOAc (400 mL×3). The combined organic layers were washed with brine (400 mL×3), dried over Na₂SO₄, filtered, and the filtrate was concentrated under reduced pressure to afford the title compound as a single enantiomer. ¹H NMR: (400 MHz, CDCl₃) δ=4.21-4.02 (m, 2H), 1.86-1.77 (m, 1H), 1.27-1.22 (m, 15H), 1.14-1.08 (m, 1H), 1.07-1.00 (m, 1H), 0.46-0.35 (m, 1H).

### SFC Analytical Method:

| Instrument | SHIMADZU LC-4D_{XR} | | | |
|---|---|---|---|---|
| column | Chiralpak IG-3 100 × 4.6 mm I.D., 3 um | | | |
| Mobile Phase | Phase A for | | | Phase C for ACN with |
| | Water with | | | 0.01875% TFA |
| | 0.0375% TFA | | | |
| | Time (min) | A(%) | B(%) | |
| Gradient | 0.01 | 60 | 40 | |
| | 10.00 | 60 | 40 | |

| | | |
|---|---|---|
| Flow rate | 1.0 | mL/min |
| Column Temp | 35° | C. |
| UV | 220 | nm |
| Retention time | 3.196 | min (the first peak: desired enantiomer) |
| | 3.620 | min (the second peak: undesired enantiomer) |

### Intermediate 9: ethyl (1S,2R)-2-(imidazo[1,2-b]pyridazin-2-yl)cyclopropane-1-carboxylate

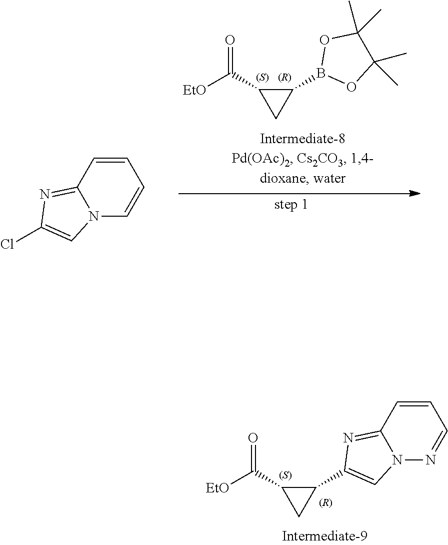

Step 1. To a solution of 2-chloroimidazo[1,2-b]pyridazine (700 mg, 4.56 mmol, 1.0 eq) and ethyl (1S,2R)-2-(imidazo[1,2-b]pyridazin-2-yl)cyclopropane-1-carboxylate (Intermediate-8) (1.31 g, 5.47 mmol, 1.2 eq) in 1,4-dioxane (10 mL) was added Pd(OAc)₂ (102 mg, 456 μmol, 0.1 eq), Cs₂CO₃ (2.97 g, 9.12 mmol, 2.0 eq), H₂O (821 mg, 45.58 mmol, 10 eq) and di(adamantan-1-yl)(butyl)phosphane (327 mg, 912 μmol, 0.2 eq), and the resulting mixture was stirred at 100° C. overnight under N₂ atmosphere. The reaction mixture was filtered and the filtrate was concentrated under reduced pressure. The residue was purified by silica gel chromatography (EtOAc/PE) to afford the title compound. LCMS: 232.2 [M+H]⁺.

### Intermediate-11: (1S,2R)-2-(5-methylpyridin-2-yl)cyclopropane-1-carboxamide

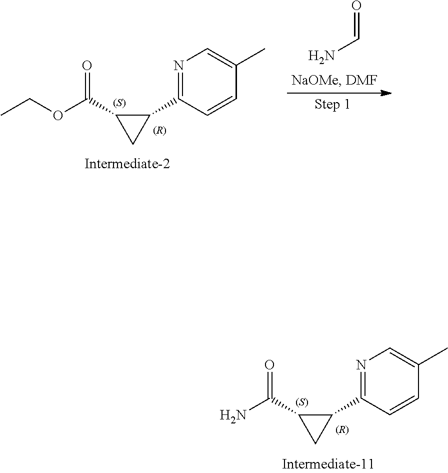

Step 1. To a solution of ethyl (1S,2R)-2-(5-methylpyridin-2-yl)cyclopropane-1-carboxylate (Intermediate-2) (2.9 g, 14.13 mmol, 1.0 eq) in anhydrous DMF (30 mL) was added NaOMe (1.53 g, 28.26 mmol, 2.0 eq) and formamide (3.82 g, 84.77 mmol, 6.0 eq) and the resulting mixture was stirred at room temperature for 1 h. The reaction mixture was poured into DCM (20 mL), the resulting suspension was filtered, the filtrate was concentrated under reduced pressure to give a residue. The residue was purified by reverse phase HPLC (C18 column, water (0.1% FA)-ACN to afford the title compound. LCMS: 199.2 [M+Na]⁺.

### Intermediate-12: 5-chloro-4-methoxypyrazolo[1,5-c]pyrimidin-2-amine

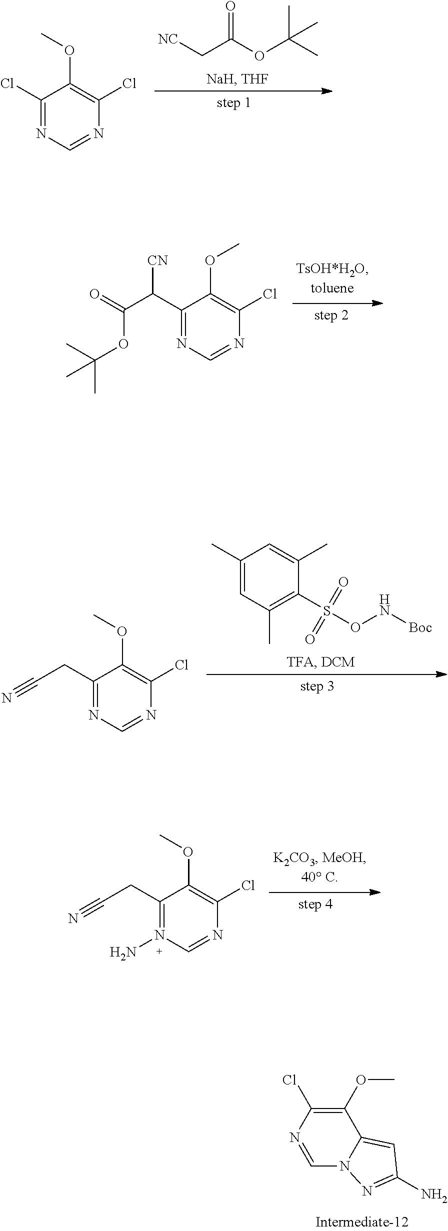

Step 1: Synthesis of tert-butyl 2-(6-chloro-5-methoxypyrimidin-4-yl)-2-cyanoacetate. To a solution of 4,6-dichloro-5-methoxypyrimidine (50.00 g, 279.32 mmol, 1.0 eq) and tert-butyl 2-cyanoacetate (98.58 g, 698.31 mmol, 2.5 eq) in anhydrous THF (500 mL) was added NaH (27.93 g, 698.31 mmol, 60% in mineral oil, 2.5 eq) at 0° C. in portions under N₂ atmosphere and the resulting mixture was stirred at room temperature for 2 h. The reaction mixture was quenched with saturated aqueous NH₄Cl (500 mL) and extracted with EtOAc (500 mL×3). The combined organic layers were washed with brine (200 mL), dried over anhydrous Na₂SO₄, filtered, and the filtrate was concentrated under reduced pressure. The residue was purified by silica gel chromatography (EtOAc/PE) to afford the title compound. LCMS: 284.0 [M+H]⁺.

Step 2: Synthesis of 2-(6-chloro-5-methoxypyrimidin-4-yl)acetonitrile. To a solution of tert-butyl 2-(6-chloro-5-methoxypyrimidin-4-yl)-2-cyanoacetate (53.00 g, 186.81 mmol, 1.0 eq) in toluene (500 mL) was added TsOH·H₂O (35.53 g, 186.81 mmol, 1.0 eq) and the resulting mixture was stirred at 105° C. for 0.5 h. The reaction mixture was diluted with H₂O (500 mL) and extracted with EtOAc (500 mL×3). The combined organic layers were washed with brine (300 mL), dried over anhydrous Na₂SO₄, filtered and the filtrate was concentrated under reduced pressure. The residue was purified by silica gel chromatography (EtOAc/PE) to afford the title compound. LCMS: 183.9 [M+H]⁺.

Step 3: Synthesis of 1-amino-4-chloro-6-(cyanomethyl)-5-methoxypyrimidin-1-ium. Tert-butyl ((mesitylsulfonyl)oxy)carbamate (70.86 g, 224.68 mmol, 2.5 eq) was dissolved in TFA (165.00 mL) portionwise at 0° C. and the resulting mixture was stirred at 0° C. for 1 h. The reaction mixture was poured into ice water (1000 mL). The resulting suspension was filtered, the filter cake was washed with water (20 mL×3). The filter cake was dissolved in DCM (200 mL), dried over anhydrous Na₂SO₄ and filtered. The filtrate was cooled to 0° C. and 2-(6-chloro-5-methoxypyrimidin-4-yl)acetonitrile (16.50 g, 89.87 mmol, 1.0 eq) was added at 0° C. The reaction mixture was stirred at room temperature overnight. The reaction mixture was filtered and the filter cake was washed with DCM (30 mL×3) to afford the title compound, which was used in the next step without further purification. LCMS: 198.9 [M]⁺.

Step 4: Synthesis of 5-chloro-4-methoxypyrazolo[1,5-c]pyrimidin-2-amine. To a suspension of 1-amino-4-chloro-6-(cyanomethyl)-5-methoxypyrimidin-1-ium (35.85 g, 89.88 mmol, 1.0 eq) in MeOH (500 mL) was added K₂CO₃ (24.84 g, 179.76 mmol, 2.0 eq) and the resulting mixture was stirred at 40° C. overnight. The reaction mixture was diluted with H₂O (500 mL) and extracted with EtOAc (500 mL×3). The combined organic layers were washed with brine (200 mL), dried over anhydrous Na₂SO₄, filtered and the filtrate was concentrated under reduced pressure. The residue was purified by reverse phase HPLC (C18 column, water (0.1% FA)-ACN to afford the title compound. LCMS: 198.9 [M+H]⁺.

### Intermediate-13: (1S,2R)—N-(5-chloro-4-methoxypyrazolo[1,5-c]pyrimidin-2-yl)-2-(5-methylpyridin-2-yl)cyclopropane-1-carboxamide

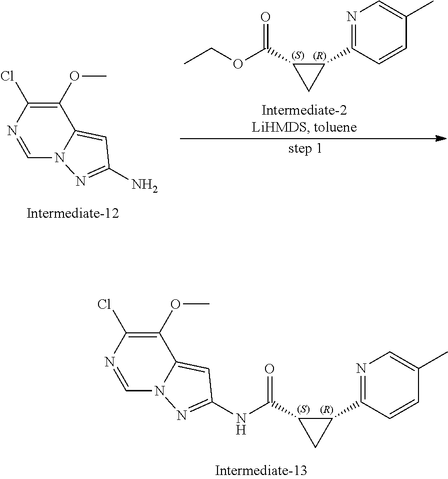

Step 1. To a solution of 5-chloro-4-methoxypyrazolo[1,5-c]pyrimidin-2-amine (Intermediate-12) (300 mg, 1.51 mmol, 1.0 eq) and ethyl (1S,2R)-2-(5-methylpyridin-2-yl)cyclopropane-1-carboxylate (Intermediate-2) (310 mg, 1.51 mmol, 1.0 eq) in toluene (5 mL) was added LiHMDS (1 M in THF, 3.02 mL, 2.0 eq) dropwise at 0° C. under N₂ atmosphere. The resulting mixture was stirred at 0° C. for 1 h. The reaction mixture was quenched with saturated aqueous NH₄Cl (25 mL) and extracted with EtOAc (30 mL×3). The combined organic layers were washed with brine (20 mL), dried over anhydrous Na₂SO₄, filtered and the filtrate was concentrated under reduced pressure. The residue was purified by reverse phase HPLC (C18 column, water (0.1% FA)-ACN to afford the title compound. LCMS: 357.9 [M+H]⁺.

### Intermediate-14: (1S,2R)—N-(5-(1-(difluoromethyl)-4-hydroxy-1H-pyrazol-5-yl)-4-hydroxypyrazolo[1,5-c]pyrimidin-2-yl)-2-(5-methylpyridin-2-yl)cyclopropane-1-carboxamide

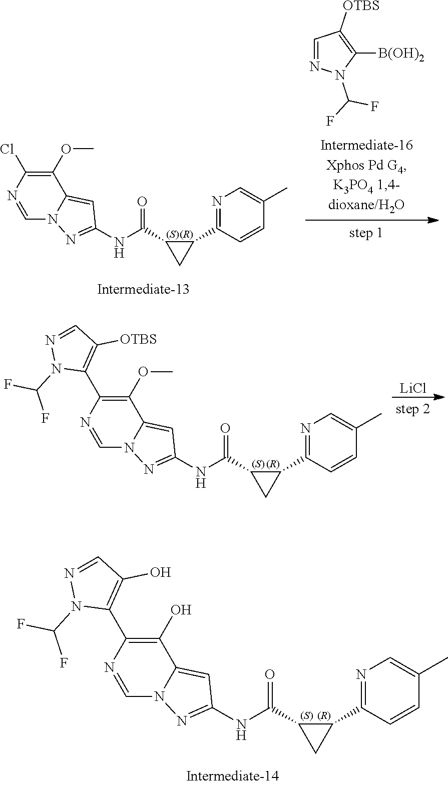

Step 1: Synthesis of (1S,2R)—N-(5-(4-((tert-butyldimethylsilyl)oxy)-1-(difluoromethyl)-1H-pyrazol-5-yl)-4-methoxypyrazolo[1,5-c]pyrimidin-2-yl)-2-(5-methylpyridin-2-yl)cyclopropane-1-carboxamide. To a solution of (1S,2R)—N-(5-chloro-4-methoxypyrazolo[1,5-c]pyrimidin-2-yl)-2-(5-methylpyridin-2-yl)cyclopropane-1-carboxamide (Intermediate-13) (130 mg, 363 μmol, 1.0 eq) and (4-((tert-butyldimethylsilyl)oxy)-1-(difluoromethyl)-1H-pyrazol-5-yl)boronic acid (Intermediate-16) (318 mg, 1.09 mmol, 3.0 eq) in 1,4-dioxane (5 mL) and H₂O (1 mL) was added K₃PO₄ (231 mg, 1.09 mmol, 3.0 eq) and XPhos Pd G4 (94 mg, 109 μmol, 0.3 eq). The resulting mixture was degassed with N₂ and stirred at 45° C. for 1 h. The reaction mixture was diluted with H₂O (10 ml) and extracted with EtOAc (15 mL×2). The combined organic layers were washed with brine (10 mL), dried over anhydrous Na₂SO₄, filtered, and the filtrate was concentrated under reduced pressure. The residue was purified by silica gel chromatography (EtOAc/PE) to afford the title compound. LCMS: 570.2 [M+H]⁺.

Step 2: Synthesis of (1S,2R)—N-(5-(1-(difluoromethyl)-4-hydroxy-1H-pyrazol-5-yl)-4-hydroxypyrazolo[1,5-c]pyrimidin-2-yl)-2-(5-methylpyridin-2-yl)cyclopropane-1-carboxamide. To a solution of (1S,2R)—N-(5-(4-((tert-butyldimethylsilyl)oxy)-1-(difluoromethyl)-1H-pyrazol-5-yl)-4-methoxypyrazolo[1,5-c]pyrimidin-2-yl)-2-(5-methylpyridin-2-yl)cyclopropane-1-carboxamide (100 mg, 176 μmol, 1.0 eq) in DMF (1 mL) was added LiCl (37 mg, 878 μmol, 5.0 eq) and the resulting mixture was stirred at 140° C. for 20 min. The reaction mixture was filtered and the filtrate was purified by reverse phase HPLC (C18 column, water (0.1% FA)-ACN to afford the title compound. LCMS: 442.2 [M+H]⁺.

### Intermediate-15: 5-bromo-4-((tert-butyldimethylsilyl)oxy)-1-(difluoromethyl)-1H-pyrazole

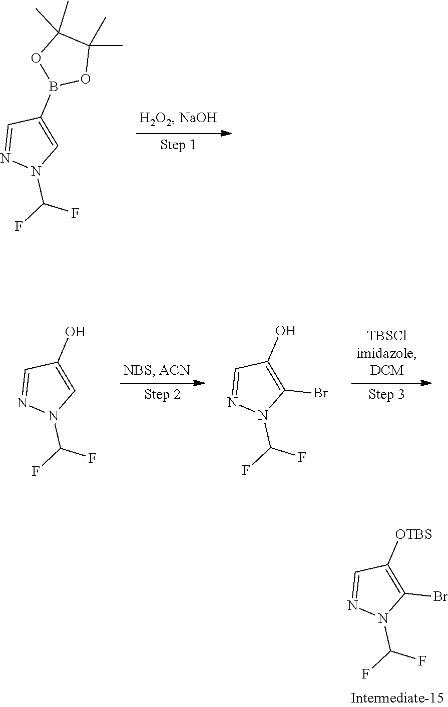 [SMILES: CC1(C)OB(c2cnn(C(F)F)c2)OC1(C)C ; Oc1cnn(C(F)F)c1 ; Oc1cnn(C(F)F)c1Br ; CC(C)(C)[Si](C)(C)Oc1cnn(C(F)F)c1Br]

Step 1. Synthesis of 1-(difluoromethyl)-1H-pyrazol-4-ol. To a solution of 1-(difluoromethyl)-4-(4,4,5,5-tetramethyl-1,3,2-dioxaborolan-2-yl)-1H-pyrazole (50.00 g, 204.88 mmol, 1.0 eq) in THF (400.00 mL) was added dropwise aqueous NaOH solution (2.0 M, 102.44 mL, 1.0 eq) at 0° C., followed by dropwise addition of H₂O₂ (49.21 g, 434.02 mmol, 41.70 mL, 30% in water, 2.12 eq) at 0° C. The resulting mixture was stirred at 0° C. for 3 h. The reaction mixture was quenched by addition of saturated aqueous Na₂SO₃ solution (250 mL) at 0° C., the mixture was diluted with water (300 mL) and then extracted with EtOAc (300 mL×3). The combined organic layers were washed with brine (300 mL), dried over anhydrous Na₂SO₄, filtered and the filtrate was concentrated under reduced pressure. The residue was purified by silica gel chromatography (EtOAc/PE) to afford the title compound. LCMS (ESI): 135.2 [M+H⁺].

Step 2. Synthesis of 5-bromo-1-(difluoromethyl)-1H-pyrazol-4-ol. To a solution of 1-(difluoromethyl)-1H-pyrazol-4-ol (15.00 g, 111.87 mmol, 1.0 eq) in MeCN (150 mL) was added NBS (21.90 g, 123.06 mmol, 1.1 eq), the resulting mixture was stirred at room temperature for 1.5 h. The reaction mixture was diluted with water (200 mL) and extracted with EtOAc (100 mL×3). The combined organic layers were washed with brine (200 mL×2), dried over anhydrous Na₂SO₄, filtered and the filtrate was concentrated under reduced pressure. The residue was purified by silica gel chromatography (EtOAc/PE) to afford the title compound. LCMS (ESI): 213.1/215.1 [M+H⁺].

Step 3. Synthesis of 5-bromo-4-((tert-butyldimethylsilyl)oxy)-1-(difluoromethyl)-1H-pyrazole. To a solution of 5-bromo-1-(difluoromethyl)-1H-pyrazol-4-ol (17.00 g, 79.82 mmol, 1.0 eq) in DCM (150 mL) was added imidazole (8.15 g, 119.73 mmol, 1.5 eq) and TBSCl (15.64 g, 103.77 mmol, 1.3 eq) and the resulting mixture was stirred at room temperature for 2 h. The reaction mixture was diluted with water (200 mL) and extracted with DCM (100 mL×3). The combined organic layers were washed with brine (100 mL), dried over anhydrous Na₂SO₄, filtered and the filtrate was concentrated under reduced pressure. The residue was purified by silica gel chromatography (EtOAc/PE) to afford the title compound. LCMS (ESI): 327.1/329.1 [M+H⁺].

### Intermediate-16: (4-((tert-butyldimethylsilyl)oxy)-1-(difluoromethyl)-1H-pyrazol-5-yl)boronic acid

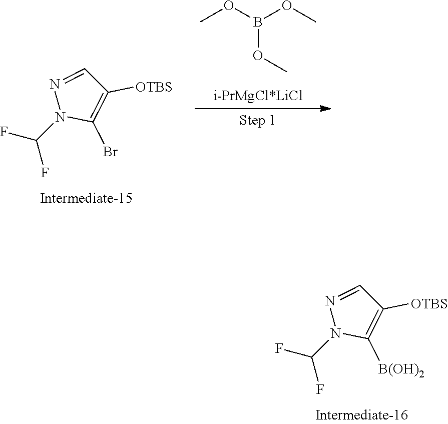

Step 1. To a solution of 5-bromo-4-((tert-butyldimethylsilyl)oxy)-1-(difluoromethyl)-1H-pyrazole (Intermediate-15) (30.00 g, 91.68 mmol, 1.0 eq) in anhydrous THF (500 mL) was added dropwise i-PrMgCl·LiCl (1.3 M in THF, 105.78 mL, 1.5 eq) at −78° C. under N₂ atmosphere and the reaction mixture was stirred at −78° C. for 0.5 h. Then trimethyl borate (38.10 g, 366.70 mmol, 4.0 eq) was added dropwise at −78° C. After addition was complete, the cooling bath was removed and the resulting mixture was stirred at room temperature for 1 h. The reaction mixture was quenched with saturated aqueous NH₄Cl (1000 mL) at 0° C. and then extracted with EtOAc (500 mL×3). The combined organic layers were washed with brine (300 mL), dried over anhydrous Na₂SO₄, filtered, and the filtrate was concentrated under reduced pressure. The residue was purified by silica gel chromatography (EtOAc/PE), the resulting crude product was triturated in PE (100 mL) and filtered, the filter cake was dried under reduced pressure to afford the title compound. LCMS: 293.0 [M+H]⁺.

### Intermediate-17: 4-(3-((tert-butyldimethylsilyl)oxy)propyl)-1-methyl-5-(4,4,5,5-tetramethyl-1,3,2-dioxaborolan-2-yl)-1H-pyrazole

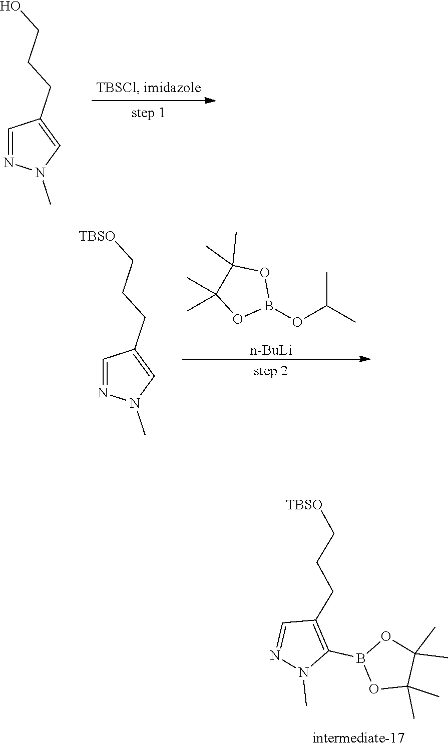

Step 1. Synthesis of 4-(3-((tert-butyldimethylsilyl)oxy)propyl)-1-methyl-1H-pyrazole. To a solution of 3-(1-methyl-1H-pyrazol-4-yl)propan-1-ol (5.00 g, 35.67 mmol, 1.0 eq) and imidazole (7.28 g, 107.00 mmol, 3.00 eq) in DCM (50 mL) was added TBSCl (10.75 g, 71.34 mmol, 2.0 eq) and the resulting mixture was stirred at room temperature for 0.5 h. The reaction mixture was diluted with H₂O (50 ml) and extracted with EtOAc (50 mL×3). The combined organic layers were washed with brine (50 mL), dried over anhydrous Na₂SO₄, filtered and the filtrate was concentrated under reduced pressure. The residue was purified by silica gel chromatography (EtOAc/PE) to afford the title compound. LCMS: 255.1 [M+H]⁺.

Step 2. Synthesis of 4-(3-((tert-butyldimethylsilyl)oxy)propyl)-1-methyl-5-(4,4,5,5-tetramethyl-1,3,2-dioxaborolan-2-yl)-1H-pyrazole. To a solution of 4-(3-((tert-butyldimethylsilyl)oxy)propyl)-1-methyl-1H-pyrazole (5.00 g, 19.65 mmol, 1.0 eq) in anhydrous THF (50 mL) was added dropwise n-BuLi (2.5 M in hexane, 11.79 mL, 1.5 eq) at −60° C. under N₂ atmosphere and the resulting mixture was stirred at −60° C. for 0.5 h. Then, 2-isopropoxy-4,4,5,5-tetramethyl-1,3,2-dioxaborolane (5.48 g, 29.48 mmol, 1.5 eq) was added dropwise at −60° C. After addition was complete, the cooling bath was removed and the resulting mixture was stirred at room temperature for 0.5 h. The reaction mixture was quenched with saturated aqueous NH₄Cl (50 mL) and extracted with EtOAc (50 mL×3). The combined organic layers were washed with brine (50 mL), dried over anhydrous Na₂SO₄, filtered and the filtrate was concentrated under reduced pressure. The residue was purified by silica gel chromatography (EtOAc/PE) to afford the title compound. LCMS: 381.1 [M+H]⁺.

### Intermediate-18: 4-(2-((tert-butyldimethylsilyl)oxy)ethoxy)-1-methyl-5-(4,4,5,5-tetramethyl-1,3,2-dioxaborolan-2-yl)-1H-pyrazole

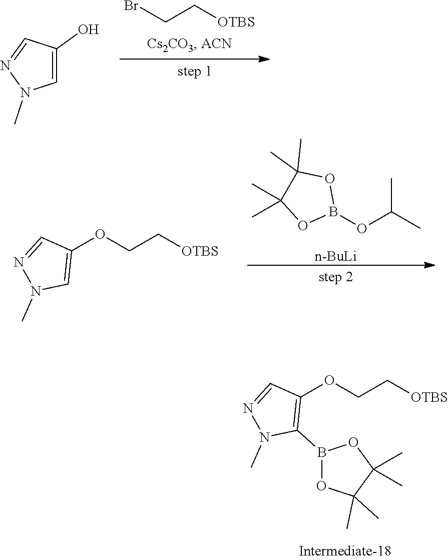

Step 1: Synthesis of 4-(2-((tert-butyldimethylsilyl)oxy)ethoxy)-1-methyl-1H-pyrazole. To a solution 1-methyl-1H-pyrazol-4-ol (20.00 g, 203.87 mmol, 1.0 eq) in ACN (200 mL) was added (2-bromoethoxy)(tert-butyl)dimethylsilane (58.52 g, 245 mmol, 1.2 eq) and Cs₂CO₃ (199.27 g, 611.60 mmol, 3.0 eq) and the resulting mixture was stirred at 80° C. for 2 h. The reaction mixture was filtered and the filtrate was concentrated under reduced pressure. The residue was purified by silica gel chromatography (EtOAc/PE) to afford the title compound. LCMS: 257.2 [M+H]⁺.

Step 2: Synthesis of 4-(2-((tert-butyldimethylsilyl)oxy)ethoxy)-1-methyl-5-(4,4,5,5-tetramethyl-1,3,2-dioxaborolan-2-yl)-1H-pyrazole. To a solution of 4-(2-((tert-butyldimethylsilyl)oxy)ethoxy)-1-methyl-1H-pyrazole (20.00 g, 78.00 mmol, 1.0 eq) in anhydrous THF (200 mL) was added dropwise n-BuLi (2.5 M in n-hexane, 46.80 mL, 1.5 eq) at −60° C. under N₂ atmosphere and the resulting mixture was stirred at −60° C. for 0.5 h. Then, 2-isopropoxy-4,4,5,5-tetramethyl-1,3,2-dioxaborolane (21.77 g, 117.00 mmol, 1.5 eq) was added dropwise at −60° C. and the resulting mixture was stirred at −60° C. for 0.5 h. The reaction was quenched with saturated NH₄Cl (250 mL) and extracted with EtOAc (200 mL×3). The combined organic layers were washed with brine (200 mL), dried over anhydrous Na₂SO₄, filtered and the filtrate was concentrated under reduced pressure. The residue was purified by silica gel chromatography (EtOAc/PE) to afford the title compound. LCMS: 383.1 [M+H]⁺.

### Intermediate-19: 6-bromo-5-chloroimidazo[1,2-a]pyrazin-2-amine

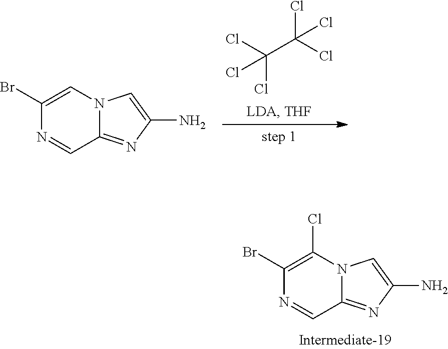 [SMILES: Nc1cn2cc(Br)ncc2n1 ; ClC(Cl)(Cl)C(Cl)(Cl)Cl ; Nc1cn2c(Cl)c(Br)ncc2n1]

Step 1. To a solution of 6-bromoimidazo[1,2-a]pyrazin-2-amine (3.50 g, 16.43 mmol, 1.0 eq) in anhydrous THF (35 mL) was added dropwise LDA (2 M in heptane and THF, 28.75 mL, 3.5 eq) at −65° C. under N₂ atmosphere, and it was stirred at −65° C. for 0.5 h. A solution of perchloroethane (3.89 g, 16.43 mmol, 1.0 eq) in THF (5 mL) was added dropwise to the reaction mixture at −65° C., and it was stirred at −65° C. for 0.5 h. The reaction mixture was quenched with saturated NH₄Cl (200 mL) and extracted with EtOAc (200 mL×3). The combined organic layers were washed with brine (100 mL), dried over anhydrous Na₂SO₄, filtered and the filtrate was concentrated under reduced pressure. The residue was purified by silica gel chromatography (EtOAc/PE) to afford the title compound. LCMS: 248.8 [M+H]⁺.

### Intermediate-20: Synthesis of 11-methyl-6,7-dihydro-11H-imidazo[1,2-a]pyrazolo[3′,4′:7,8][1,4]dioxocino[6,5-e]pyrazin-2-amine

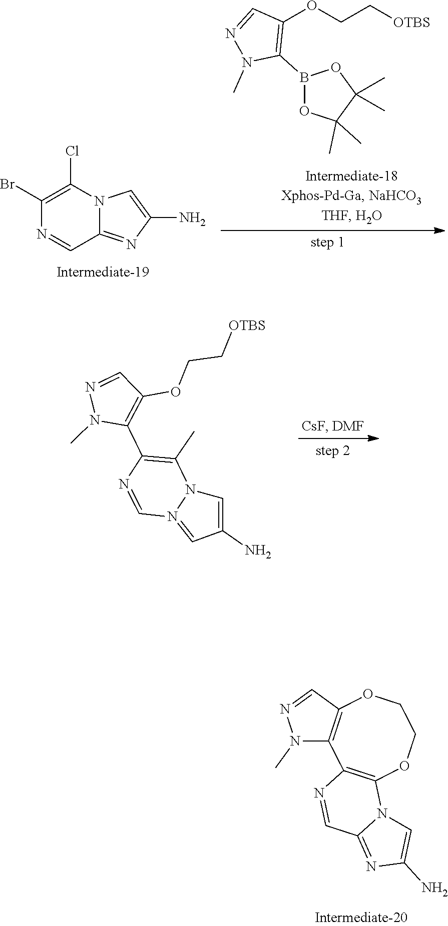

Step 1: Synthesis of 6-(4-(2-((tert-butyldimethylsilyl)oxy)ethoxy)-1-methyl-1H-pyrazol-5-yl)-5-chloroimidazo[1,2-a]pyrazin-2-amine. To a solution of 6-bromo-5-chloroimidazo[1,2-a]pyrazin-2-amine (Intermediate-19) (80 mg, 323 μmol, 1.0 eq) and 4-(2-((tert-butyldimethylsilyl)oxy)ethoxy)-1-methyl-5-(4,4,5,5-tetramethyl-1,3,2-dioxaborolan-2-yl)-1H-pyrazole (Intermediate-18) (742 mg, 1.94 mmol, 6.0 eq) in THF (5 mL) and H₂O (1 mL) was added XPhos Pd G4 (42 mg, 48 μmol, 0.15 eq) and NaHCO₃ (81 mg, 970 μmol, 3.0 eq), and the resulting mixture was stirred at 60° C. for 1 h under N₂ atmosphere. The reaction mixture was diluted with water (10 mL) and extracted with EtOAc (10 mL×3). The combined organic layers were washed with brine (10 mL), dried over anhydrous Na₂SO₄, filtered, and the filtrate was concentrated under reduced pressure. The residue was purified by silica gel chromatography (EtOAc/PE) to afford the title compound. LCMS: 423.1 [M+H]⁺.

Step 2: Synthesis of 11-methyl-6,7-dihydro-11H-imidazo[1,2-a]pyrazolo[3′,4′:7,8][1,4]dioxocino[6,5-e]pyrazin-2-amine. To a solution of 6-(4-(2-((tert-butyldimethylsilyl)oxy)ethoxy)-1-methyl-1H-pyrazol-5-yl)-5-chloroimidazo[1,2-a]pyrazin-2-amine (30 mg, 71 μmol, 1.0 eq) in DMF (1 mL) was added CsF (54 mg, 355 μmol, 5.0 eq) and the resulting mixture was stirred at 100° C. for 7 h. The reaction mixture was diluted with H₂O (10 mL), then pH was adjusted to 14 with aqueous NaOH solution (1 N). The resulting mixture was extracted with DCM (10 mL×2), the combined organic layers were washed with brine (10 mL), dried over anhydrous Na₂SO₄, filtered, and the filtrate was concentrated under reduced pressure. The residue was purified by preparative TLC (EtOAc/PE) to afford the title compound. LCMS: 273.0 [M+H]⁺.

### Intermediate-21: (1S,2R)-2-(5-methylpyridin-2-yl)cyclopropane-1-carboxylic acid

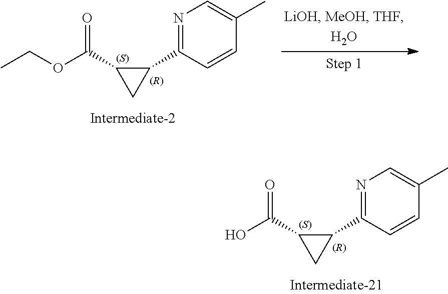

Step 1. To a solution of ethyl (1S,2R)-2-(5-methylpyridin-2-yl)cyclopropane-1-carboxylate (Intermediate-2) (1.40 g, 6.82 mmol, 1.0 eq) in THF (3 mL), MeOH (3 mL) and H₂O (3 mL) was added LiOH·H₂O (716 mg, 17.05 mmol, 2.5 eq) and the resulting mixture was stirred at room temperature for 1 h. The reaction mixture was adjusted to pH 6 with aqueous HCl (6 N) and then concentrated under reduced pressure to remove MeOH and THF, the resulting aqueous solution was purified directly by reverse phase HPLC (C18 column, water (0.1% FA)-ACN to afford the title compound. LCMS: 178.0 [M+H]⁺.

### Intermediate-22: (1S,2R)—N-(5-chloro-4-hydroxypyrazolo[1,5-a]pyridin-2-yl)-2-(5-methylpyridin-2-yl)cyclopropane-1-carboxamide

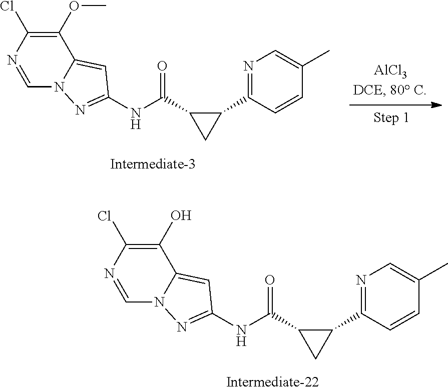 [SMILES: COc1c(Cl)ncn2nc(NC(=O)[C@H]3C[C@H]3c3ccc(C)cn3)cc12 ; Cc1ccc([C@@H]2C[C@@H]2C(=O)Nc2cc3c(O)c(Cl)ncn3n2)nc1]

Step 1. To a solution of (1S,2R)—N-(5-chloro-4-methoxypyrazolo[1,5-a]pyridin-2-yl)-2-(5-methylpyridin-2-yl)cyclopropane-1-carboxamide (Intermediate-3) (1.00 g, 2.80 mmol, 1 eq) in DCE (15 mL) was added AlCl₃ (1.87 g, 14.01 mmol, 5 eq) at 0° C. under N₂ atmosphere. The resulting mixture was stirred at 80° C. for 3 h, then cooled down to 0° C. and H₂O (2 mL) and MeOH (25 mL) were added. The resulting mixture was filtered and the filtrate was concentrated under reduced pressure. The residue was purified by flash silica gel chromatography (Eluent of MeOH/DCM) to afford the title compound. LCMS: 342.9 [M+H⁺].

### Intermediate-23: (4-((tert-butoxycarbonyl)amino)-1-methyl-1H-pyrazol-5-yl)boronic acid

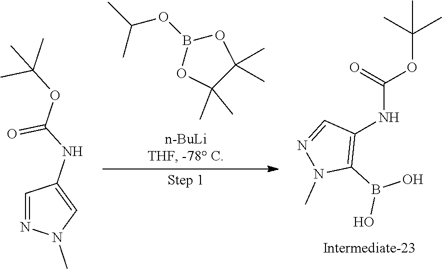 [SMILES: Cn1cc(NC(=O)OC(C)(C)C)cn1 ; CC(C)OB1OC(C)(C)C(C)(C)O1 ; Cn1ncc(NC(=O)OC(C)(C)C)c1B(O)O]

Step 1. To a solution of tert-butyl (1-methyl-1H-pyrazol-4-yl)carbamate (1.00 g, 5.07 mmol, 1 eq) and 2-isopropoxy-4,4,5,5-tetramethyl-1,3,2-dioxaborolane (1.13 g, 6.08 mmol, 1.24 mL, 1.2 eq) in THF (10 mL) was added n-BuLi (2.5 M in hexane, 4.46 mL, 2.2 eq) dropwise at −70° C. under N₂ atmosphere. The mixture was stirred at −70° C. for 0.5 h, then warmed up to 25° C., quenched with H₂O (20 mL) and washed with EtOAc (25 mL×2). The aqueous phase was separated and lyophilized to afford the title compound. LCMS: 242.0 [M+H⁺].

### Intermediate-24: 2-((5-(4-amino-1-methyl-1H-pyrazol-5-yl)-2-((1S,2R)-2-(5-methylpyridin-2-yl)cyclopropane-1-carboxamido)pyrazolo[1,5-a]pyridin-4-yl)oxy)acetic acid hydrochloride

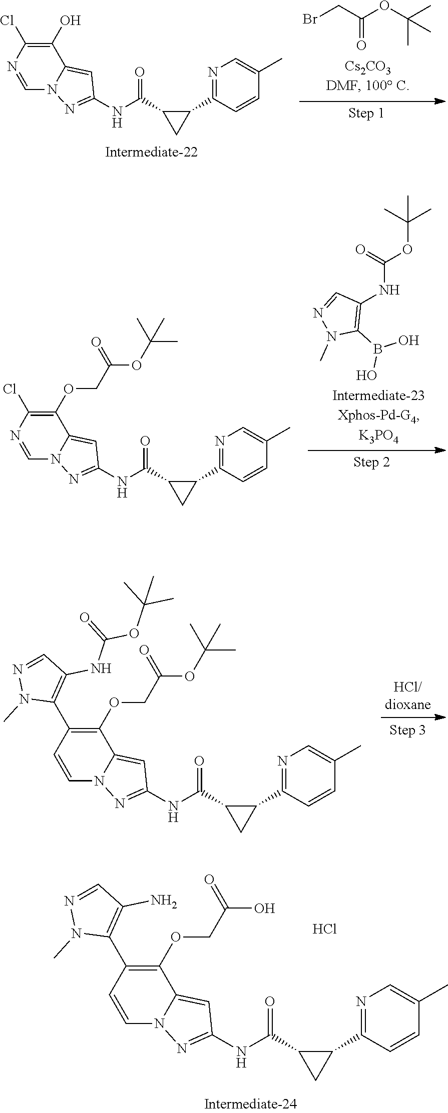

Step 1. Synthesis of tert-butyl 2-((5-chloro-2-((1S,2R)-2-(5-methylpyridin-2-yl)cyclopropane-1-carboxamido)pyrazolo[1,5-a]pyridin-4-yl)oxy)acetate. To a solution of (1S,2R)—N-(5-chloro-4-hydroxypyrazolo[1,5-a]pyridin-2-yl)-2-(5-methylpyridin-2-yl)cyclopropane-1-carboxamide (Intermediate-22) (1.00 g, 2.92 mmol, 1 eq) and tert-butyl 2-bromoacetate (0.57 g, 2.91 mmol, 1 eq) in DMF (10 mL) was added Cs₂CO₃ (3.80 g, 11.67 mmol, 4 eq). The resulting mixture was stirred at 85° C. for 0.5 h. After cooling down to room temperature the reaction mixture was diluted with H₂O (20 mL) and extracted with DCM (25 mL×2). The combined organic phase was washed with brine (10 mL), dried over anhydrous Na₂SO₄, filtered and concentrated under reduced pressure. The residue was purified by reverse phase preparative HPLC (ACN/H₂O, 0.01% NH₃·H₂O) to afford the title compound. LCMS: 457.1 [M+H⁺].

Step 2. Synthesis of tert-butyl 2-((5-(4-((tert-butoxycarbonyl)amino)-1-methyl-1H-pyrazol-5-yl)-2-((1S,2R)-2-(5-methylpyridin-2-yl)cyclopropane-1-carboxamido)pyrazolo[1,5-a]pyridin-4-yl)oxy)acetate. To a solution of tert-butyl 2-((5-chloro-2-((1S,2R)-2-(5-methylpyridin-2-yl)cyclopropane-1-carboxamido)pyrazolo[1,5-a]pyridin-4-yl)oxy)acetate (100 mg, 219 μmol, 1 eq) and (4-((tert-butoxycarbonyl)amino)-1-methyl-1H-pyrazol-5-yl)boronic acid (Intermediate-23) (283 mg, 875 μmol, 4 eq) in dioxane (4 mL) and H₂O (1 mL) was added K₃PO₄ (186 mg, 875 μmol, 4 eq) and Xphos-Pd-G4 (19 mg, 22 μmol, 0.1 eq) under N₂ atmosphere. The reaction mixture was stirred at 40° C. for 1 h. The reaction mixture was filtered and the filtrate was concentrated under reduced pressure. The residue was purified by reverse phase preparative HPLC (ACN/H₂O, 0.05% NH₃·H₂O) to afford the title compound. LCMS: 618.4 [M+H⁺].

Step 3. Synthesis of 2-((5-(4-amino-1-methyl-1H-pyrazol-5-yl)-2-((1S,2R)-2-(5-methylpyridin-2-yl)cyclopropane-1-carboxamido)pyrazolo[1,5-a]pyridin-4-yl)oxy)acetic acid hydrochloride. To a solution of tert-butyl 2-((5-(4-((tert-butoxycarbonyl)amino)-1-methyl-1H-pyrazol-5-yl)-2-((1S,2R)-2-(5-methylpyridin-2-yl)cyclopropane-1-carboxamido)pyrazolo[1,5-a]pyridin-4-yl)oxy)acetate (110 mg, 178 μmol, 1 eq) in DCM (1 mL) was added HCl (2M in dioxane, 2 mL). The mixture was stirred at 25° C. for 0.5 h. The reaction mixture was concentrated under reduced pressure and then triturated by DCM (1 mL) to afford the title compound. LCMS: 462.1 [M+H⁺].

### Intermediate-25: (1s,3s)-cyclobutane-1,3-diyl bis(4-methylbenzenesulfonate)

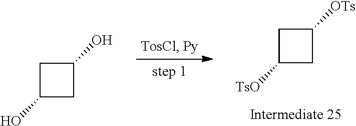 [SMILES: O[C@H]1C[C@@H](O)C1 ; Cc1ccc(S(=O)(=O)O[C@H]2C[C@@H](OS(=O)(=O)c3ccc(C)cc3)C2)cc1]

Step 1. To a solution of (1s,3s)-cyclobutane-1,3-diol (100 mg, 1.14 mmol, 1.0 eq) in pyridine (2 mL) and toluene (3 mL) was added TsCl (649 mg, 3.41 mmol, 3.0 eq) at 0° C., then the resulting mixture was stirred at room temperature for 1 h. The reaction mixture was poured into water (100 mL) and extracted with EtOAc (40 mL×2). The combined organic layers were washed with brine (30 mL), dried over anhydrous Na₂SO₄, filtered, and the filtrate was concentrated under reduced pressure. The residue was purified by silica gel chromatography (EtOAc/PE) to afford the title compound. ¹H NMR (400 MHz, CDCl₃) δ 7.78-7.70 (m, 4H), 7.34 (d, J=8.0 Hz, 4H), 4.49-4.33 (m, 2H), 2.70-2.58 (m, 2H), 2.46 (s, 6H), 2.40-2.27 (m, 2H).

### Intermediate-26: 2-(4-hydroxy-5-(4-hydroxy-1-methyl-1H-pyrazol-5-yl)pyrazolo[1,5-a]pyridin-2-yl)isoindoline-1,3-dione

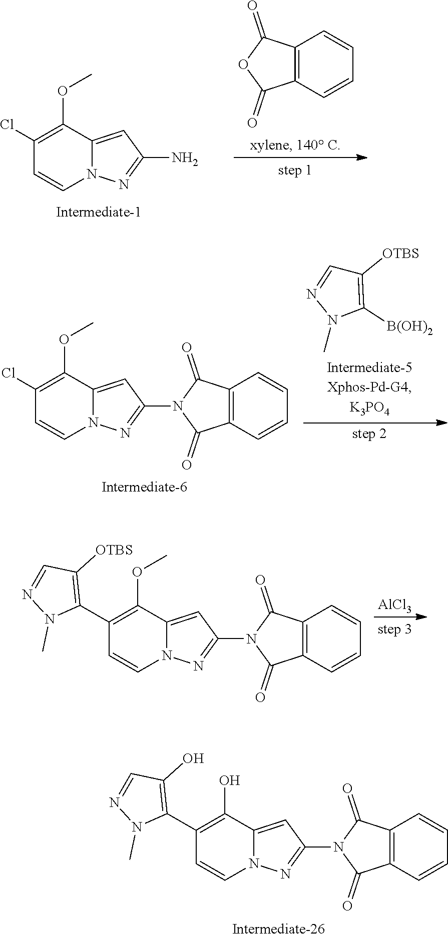

Step 1. Synthesis of 2-(5-chloro-4-methoxypyrazolo[1,5-a]pyridin-2-yl)isoindoline-1,3-dione. To a solution of 5-chloro-4-methoxy-pyrazolo[1,5-a]pyridin-2-amine (Intermediate-1) (120.00 g, 607.22 mmol, 1.0 eq) in xylene (1500 mL) was added isobenzofuran-1,3-dione (116.92 g, 789.39 mmol, 1.3 eq), and it was stirred at 140° C. overnight. The reaction mixture was cooled down and left for 8 h at room temperature. The resulting mixture was filtered, the filter cake was washed with MTBE (500 mL) and then dried under reduced pressure to afford the title compound which was used in the next step without further purification. LCMS: 327.9 [M+H]⁺.

Step 2. Synthesis of 2-(5-(4-((tert-butyldimethylsilyl)oxy)-1-methyl-1H-pyrazol-5-yl)-4-methoxypyrazolo[1,5-a]pyridin-2-yl)isoindoline-1,3-dione. To a solution of 2-(5-chloro-4-methoxypyrazolo[1,5-a]pyridin-2-yl)isoindoline-1,3-dione (Intermediate-6) (100.00 g, 305.14 mmol, 1.0 eq) and (4-((tert-butyldimethylsilyl)oxy)-1-methyl-1H-pyrazol-5-yl)boronic acid (Intermediate-5) (156.34 g, 610.27 mmol, 2.0 eq) in 1,4-dioxane (1 L) and H₂O (100 mL) was added Xphos-Pd-G4 (26.26 g, 30.51 mmol, 0.1 eq) and K₃PO₄ (129.54 g, 610.27 mmol, 2.0 eq) and the resulting mixture was stirred at 40° C. for 1 h under N₂ atmosphere. The reaction mixture was poured into water (1 L) and then extracted with EtOAc (500 mL×3). The combined organic layers were washed with brine (300 mL), dried over anhydrous Na₂SO₄, filtered and the filtrate was concentrated under reduced pressure. The residue was purified by flash silica gel chromatography (eluent of PE/EtOAc) to afford the title compound. LCMS: 504.1 [M+H]⁺.

Step 3. Synthesis of 2-(4-hydroxy-5-(4-hydroxy-1-methyl-1H-pyrazol-5-yl)pyrazolo[1,5-a]pyridin-2-yl)isoindoline-1,3-dione. To a solution of 2-(5-(4-((tert-butyldimethylsilyl)oxy)-1-methyl-1H-pyrazol-5-yl)-4-methoxypyrazolo[1,5-a]pyridin-2-yl)isoindoline-1,3-dione (140.00 g, 277.99 mmol, 1.0 eq) in DCE (1400 mL) was added AlCl₃ powder (370.67 g, 2.78 mol, 151.91 mmol, 10.0 eq) portion-wise at room temperature under N₂ atmosphere, and the resulting mixture was stirred at room temperature for 0.5 h. The reaction mixture was poured into water (3 L) slowly, the resulting suspension was filtered. The filter cake was suspended in water (2 L) and pH was adjusted to ˜6 with saturated aqueous NaOAc solution. The resulting mixture was filtered, the filter cake was washed with water (500 mL) and ACN (800 mL), and then dried under reduced pressure to afford the title compound which was used in the next step without further purification. LCMS: 376.0 [M+H]⁺.

### Intermediate-27: 11-methyl-6,7-dihydro-5H,11H-pyrazolo[1,5-a]pyrazolo[3′,4′:8,9][1,5]-dioxonino[6,7-c]pyridin-2-amine

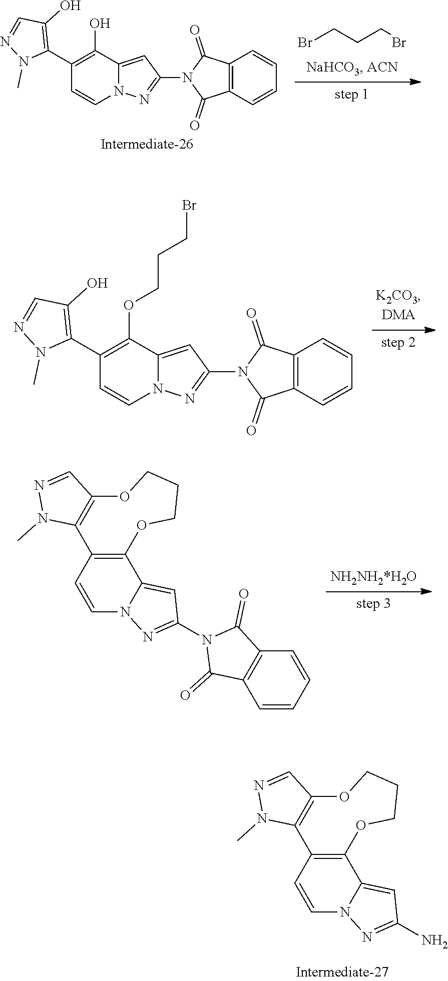

Step 1. Synthesis of 2-(4-(3-bromopropoxy)-5-(4-hydroxy-1-methyl-1H-pyrazol-5-yl)pyrazolo[1,5-a]pyridin-2-yl)isoindoline-1,3-dione. To a solution of 2-(4-hydroxy-5-(4-hydroxy-1-methyl-1H-pyrazol-5-yl)pyrazolo[1,5-a]pyridin-2-yl)isoindoline-1,3-dione (Intermediate-26) (1.00 g, 2.66 mmol, 1.0 eq) in DMA (20 mL) was added NaHCO₃ (1.12 g, 13.32 mmol, 5.0 eq) and 1,3-dibromopropane (807 mg, 4.00 mmol, 1.5 eq), the resulting mixture was stirred at 30° C. overnight. The reaction mixture was used directly in the following steps. LCMS: 497.9 [M+H]⁺.

Step 2. Synthesis of 2-(11-methyl-6,7-dihydro-5H,11H-pyrazolo[1,5-a]pyrazolo[3′,4′:8,9][1,5]dioxonino[6,7-c]pyridin-2-yl)isoindoline-1,3-dione. To the reaction mixture of step 1 was added K₂CO₃ (1.10 g, 7.99 mmol, 3.0 eq) and the resulting mixture was heated to 130° C. for 1 h. The reaction mixture was cooled down to room temperature, poured into water (20 mL) and extracted with EtOAc (20 mL×3). The combined organic layers were washed with brine (20 mL), dried over anhydrous Na₂SO₄, filtered and the filtrate was concentrated under reduced pressure to afford the title compound which was used in the next step without further purification. LCMS: 416.0 [M+H]⁺.

Step 3. Synthesis of 11-methyl-6,7-dihydro-5H,11H-pyrazolo[1,5-a]pyrazolo[3′,4′:8,9][1,5]dioxonino[6,7-c]pyridin-2-amine. To a solution of 2-(11-methyl-6,7-dihydro-5H,11H-pyrazolo[1,5-a]pyrazolo[3′,4′:8,9][1,5]dioxonino[6,7-c]pyridin-2-yl)isoindoline-1,3-dione (1.70 g, 4.09 mmol, 1.0 eq) in EtOH (10 mL) was added N₂H₄*H₂O (5.53 g, 26.5 eq), and the resulting mixture was stirred at room temperature for 1 h. The reaction mixture was poured into water (80 mL) and extracted with EtOAc (50 mL×3). The combined organic layers were washed with brine (20 mL), dried over anhydrous Na₂SO₄, filtered and the filtrate was concentrated under reduced pressure. The residue was purified by flash silica gel chromatography (eluent of PE/EtOAc) to afford the title compound. LCMS: 286.0 [M+H]⁺.

### Intermediate-28: ethyl (1S,2R)-2-(5-(difluoromethyl)pyrimidin-2-yl)cyclopropane-1-carboxylate

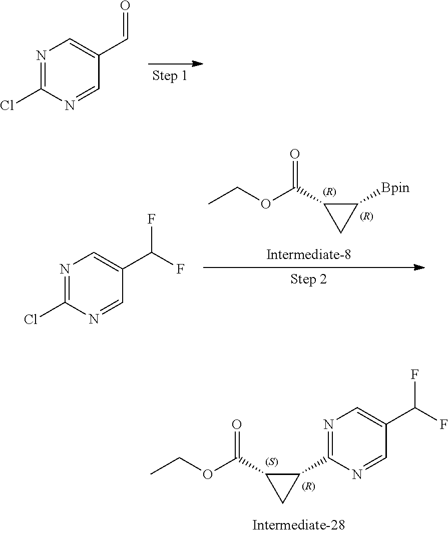

Step 1. Synthesis of 2-chloro-5-(difluoromethyl)pyrimidine. To a solution of 2-chloropyrimidine-5-carbaldehyde (10.00 g, 70.15 mmol, 1 eq) in DCM (200 mL) was added DAST (24.88 g, 154.34 mmol, 20.39 mL, 2.2 eq) at 0° C. under N₂ atmosphere. The mixture was stirred at 0° C. for 1 h and then warmed up to 25° C. and stirred for 1 h. The mixture was diluted with saturated aqueous NH₄Cl (1 L) and extracted with DCM (3×200 mL), the combined organic layer was washed with brine (1 L), dried over anhydrous Na₂SO₄, filtered and concentrated under reduced pressure. The residue was purified by flash silica gel chromatography (eluent of PE/EtOAc) to afford the title compound. LCMS: 165.2 [M+H]⁺.

Step 2. Synthesis of ethyl (1S,2R)-2-(5-(difluoromethyl)pyrimidin-2-yl)cyclopropane-1-carboxylate. To a solution of 2-chloro-5-(difluoromethyl)pyrimidine (3.80 g, 23.09 mmol, 1 eq) and ethyl (1S,2R)-2-(4,4,5,5-tetramethyl-1,3,2-dioxaborolan-2-yl)cyclopropanecarboxylate (Intermediate-8) (8.32 g, 34.64 mmol, 1.5 eq) in dioxane (100 mL) and H₂O (10 mL) was added cataCXium® A (1.66 g, 4.62 mmol, 0.2 eq), Cs₂CO₃ (15.05 g, 46.19 mmol, 2 eq) and Pd(OAc)₂ (1.04 g, 4.62 mmol, 0.2 eq) under N₂ atmosphere. The mixture was stirred at 100° C. for 16 h. The reaction mixture was filtered and the filter cake was washed with 40 mL EtOAc. The combined filtrate was concentrated under reduced pressure. The residue was purified by reverse phase preparative HPLC (MeCN/H₂O (0.1% FA)) followed by flash silica gel chromatography (eluent of DCM/MeOH) to afford the title compound. ¹H NMR (400 MHz, DMSO-d6) δ=8.91 (2H), 7.18 (1H), 3.91-3.75 (2H), 2.87 (1H), 2.36-2.19 (1H), 1.85-1.70 (1H), 1.55-1.38 (1H), 0.93 (3H) ppm. LCMS: 243.0 [M+H]⁺.

### Intermediate-29: ethyl (1S,2R)-2-(5-methylpyrimidin-2-yl)cyclopropane-1-carboxylate

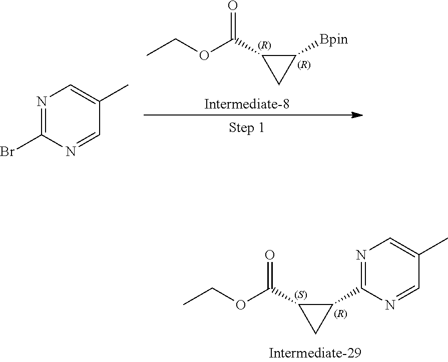

Step 1. A mixture of 2-bromo-5-methyl-pyrimidine (90 g, 520.20 mmol, 1 eq), ethyl (1S, 2R)-2-(4, 4, 5, 5-tetramethyl-1, 3, 2-dioxaborolan-2-yl) cyclopropanecarboxylate (Intermediate-8) (131.15 g, 546.21 mmol, 1.05 eq), Cs₂CO₃ (423.73 g, 1.30 mol, 2.5 eq) and cataCXium® A Pd G3 (37.88 g, 52.02 mmol, 0.1 eq) in toluene (1000 mL) and H₂O (100 mL) was degassed with N₂ and then stirred at 100° C. for 16 h under N₂ atmosphere. The reaction mixture was concentrated under reduced pressure. The residue was diluted with H₂O (1 L) and extracted with EA (500 mL×3). The combined organic layers were washed with brine (500 mL×2), dried over Na₂SO₄, filtered and the filtrate was concentrated under reduced pressure. The residue was purified by flash silica gel chromatography (eluent of EtOAc/hexane) to afford the title compound. ¹H NMR (400 MHz, CDCl₃) δ=8.46 (s, 2H), 4.03-3.83 (m, 2H), 2.82-2.73 (m, 1H), 2.25 (s, 3H), 2.19-2.12 (m, 1H), 1.98-1.94 (m, 1H), 1.47-1.40 (m, 1H), 1.08 (t, 3H) ppm. LCMS: 207.3 [M+H]⁺.

### Intermediate-30: (R)-2,7-dimethyl-2,3-dihydro-7H-pyrazolo[1,5-a]pyrazolo[3′,4′:7,8][1,4]-dioxocino[5,6-c]pyridin-12-amine and Intermediate-31: (R)-3,7-dimethyl-2,3-dihydro-7H-pyrazolo[1,5-a]pyrazolo[3′,4′:7,8][1,4]-dioxocino[5,6-c]pyridin-12-amine

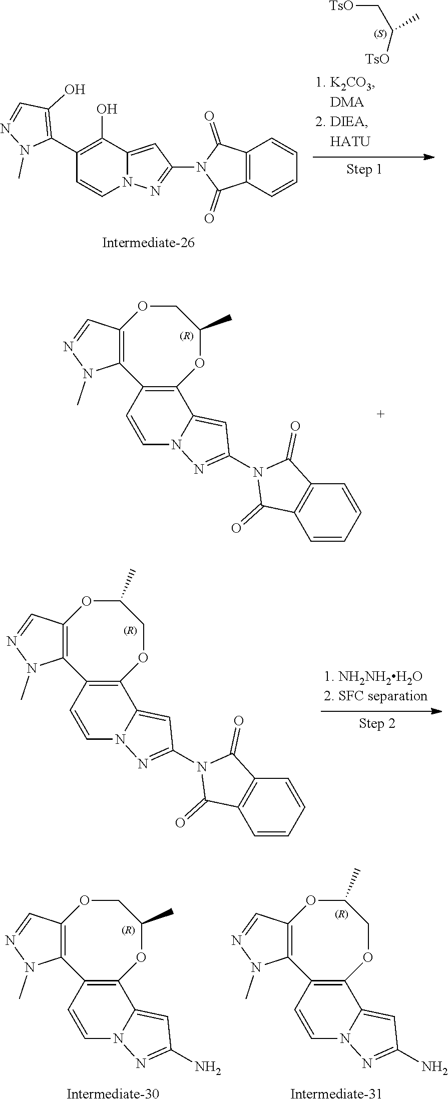

Step 1. Synthesis of (R)-2-(2,7-dimethyl-2,3-dihydro-7H-pyrazolo[1,5-a]pyrazolo[3′,4′:7,8][1,4]dioxocino[5,6-c]pyridin-12-yl)isoindoline-1,3-dione and (R)-2-(3,7-dimethyl-2,3-dihydro-7H-pyrazolo[1,5-a]pyrazolo[3′,4′:7,8][1,4]dioxocino[5,6-c]pyridin-12-yl)isoindoline-1,3-dione. To a solution of (S)-propane-1,2-diyl bis(4-methylbenzenesulfonate) (2.05 g, 5.33 mmol, 2.0 eq) and 2-(4-hydroxy-5-(4-hydroxy-1-methyl-1H-pyrazol-5-yl)pyrazolo[1,5-a]pyridin-2-yl)isoindoline-1,3-dione (Intermediate-26) (1.00 g, 2.66 mmol, 1.0 eq) in DMA (10 mL) was added K₂CO₃ (2.95 g, 21.31 mmol, 8.0 eq) and the resulting mixture was stirred at 120° C. for 1 h under N₂ atmosphere. The reaction mixture was cooled down to room temperature, HATU (1.01 g, 2.66 mmol, 1.0 eq) and DIEA (689 mg, 5.33 mmol, 2.0 eq) were added and stirring was continued at room temperature for 1 h. The resulting mixture was poured into saturated aqueous citric acid solution (50 mL) and extracted with EtOAc (30 mL×5). The combined organic phase was dried over anhydrous Na₂SO₄, filtered and the filtrate was concentrated under reduced pressure to afford the mixture of title compounds. This mixture was used in the next step without further purification. LCMS: 416.1 [M+H]⁺.

Step 2. Synthesis of (R)-2,7-dimethyl-2,3-dihydro-7H-pyrazolo[1,5-a]pyrazolo[3′,4′:7,8][1,4]dioxocino[5,6-c]pyridin-12-amine and (R)-3,7-dimethyl-2,3-dihydro-7H-pyrazolo[1,5-a]pyrazolo[3′,4′:7,8][1,4]dioxocino[5,6-c]pyridin-12-amine. To a solution of (R)-2-(2,7-dimethyl-2,3-dihydro-7H-pyrazolo[1,5-a]pyrazolo[3′,4′:7,8][1,4]dioxocino[5,6-c]pyridin-12-yl)isoindoline-1,3-dione and (R)-2-(3,7-dimethyl-2,3-dihydro-7H-pyrazolo[1,5-a]pyrazolo[3′,4′:7,8][1,4]dioxocino[5,6-c]pyridin-12-yl)isoindoline-1,3-dione (mixture as obtained in previous step 1) (1.00 g, 2.41 mmol, 1.0 eq) in EtOH (4 mL) was added hydrazine hydrate (3.73 g, 73.06 mmol, 30.35 eq) and the resulting mixture was stirred at room temperature for 0.5 h. The reaction mixture was poured into water (30 mL) and extracted with EtOAc (30 mL×3). The combined organic layers were washed with brine (30 mL), dried over anhydrous Na₂SO₄, filtered and the filtrate was concentrated under reduced pressure. The residue was purified by flash silica gel chromatography (eluent of PE/EtOAc) to afford the mixture of title compounds. LCMS: 286.0 [M+H]

The mixture of (R)-2,7-dimethyl-2,3-dihydro-7H-pyrazolo[1,5-a]pyrazolo[3′,4′:7,8][1,4]dioxocino[5,6-c]pyridin-12-amine and (R)-3,7-dimethyl-2,3-dihydro-7H-pyrazolo[1,5-a]pyrazolo[3′,4′:7,8][1,4]dioxocino[5,6-c]pyridin-12-amine (600 mg, 1.44 mmol, 1.0 eq) was separated by chiral SFC (Instrument: SHIMADZU LC-30Adsf; column: Chiralcel OJ-3 50×4.6 mm I.D., particle size 3 μm; mobile phase: phase A for CO₂, phase B for IPA (0.05% DEA); flow rate: 3.0 mL/min; retention time: peak1: 1.446 min, peak2: 1.776 min; back pressure: 100 bar to keep the CO₂ in supercritical flow; wave length: 220 nm)) to afford the title compound as peak1.

Peak1: ((R)-2,7-dimethyl-2,3-dihydro-7H-pyrazolo[1,5-a]pyrazolo[3′,4′:7,8][1,4]dioxocino[5,6-c]pyridin-12-amine, Intermediate-30): ¹H NMR (400 MHz, CDCl₃) δ=8.09 (d, J=7.2 Hz, 1H), 7.39 (s, 1H), 6.49 (d, J=7.2 Hz, 1H), 5.91 (s, 1H), 4.22 (br d, J=13.2 Hz, 2H), 3.87 (s, 3H), 3.70-3.58 (m, 1H), 1.48 (br d, J=6.4 Hz, 3H) ppm. LCMS: 286.1 [M+H].

Peak2: ((R)-3,7-dimethyl-2,3-dihydro-7H-pyrazolo[1,5-a]pyrazolo[3′,4′:7,8][1,4]dioxocino[5,6-c]pyridin-12-amine, Intermediate-31): ¹H NMR (400 MHz, CDCl₃) δ=8.08 (d, J=7.2 Hz, 1H), 7.37 (br s, 1H), 6.48 (d, J=7.2 Hz, 1H), 5.92 (s, 1H), 4.33 (br s, 2H), 3.93-3.82 (m, 3H), 3.49 (s, 1H), 1.28 (d, J=6.4 Hz, 3H) ppm. LCMS: 286.1 [M+H].

### Intermediate-32: (2S,3S)-3-hydroxybutan-2-yl 4-methylbenzenesulfonate

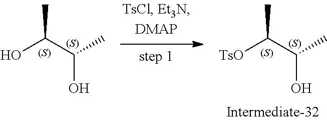

Step 1. To a solution of (2S,3S)-butane-2,3-diol (5.00 g, 55.48 mmol, 1.0 eq), TEA (19.65 g, 194.18 mmol, 3.5 eq) and DMAP (678 mg, 5.55 mmol, 0.1 eq) in DCM (50 mL) was added TsCl (12.69 g, 66.58 mmol, 1.2 eq) dropwise at 0° C. and the resulting mixture was stirred at 40° C. for 1 h. The reaction mixture was poured into water (50 mL) and extracted with DCM (30 mL×2). The combined organic layers were washed with brine (20 mL), dried over anhydrous Na₂SO₄, filtered and the filtrate was concentrated under reduced pressure. The residue was purified by flash silica gel chromatography (eluent of PE/EtOAc) to afford the title compound. ¹H NMR (400 MHz, CDCl₃) δ=7.84 (d, 2H), 7.37 (d, 2H), 4.56-4.43 (m, 1H), 3.81-3.69 (m, 1H), 2.48 (s, 3H), 1.28 (d, 3H), 1.16 (d, 3H) ppm.

### Intermediate-33: (2R,3R)-2,3,7-trimethyl-2,3-dihydro-7H-pyrazolo[1,5-a]pyrazolo-[3′,4′:7,8][1,4]dioxocino[5,6-c]pyridin-12-amine

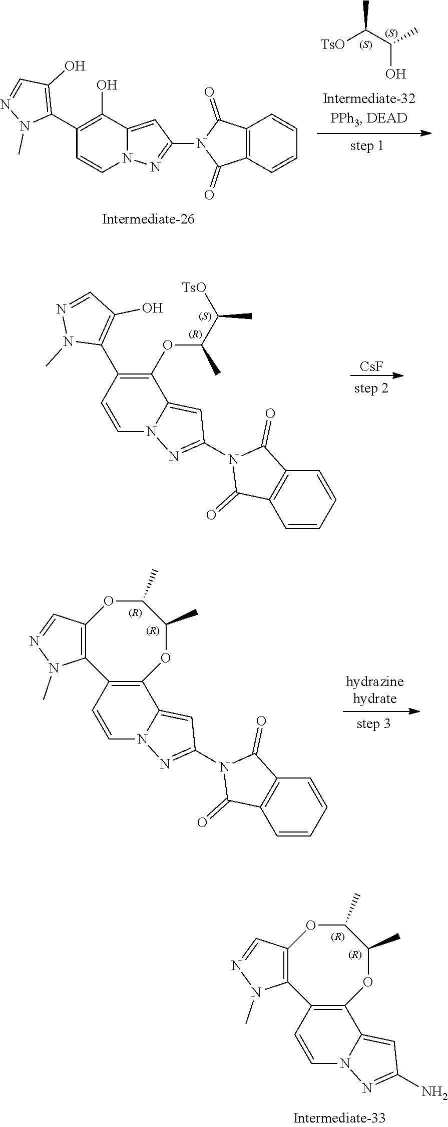

Step 1. Synthesis of (2S,3R)-3-((2-(1,3-dioxoisoindolin-2-yl)-5-(4-hydroxy-1-methyl-1H-pyrazol-5-yl)pyrazolo[1,5-a]pyridin-4-yl)oxy)butan-2-yl 4-methylbenzenesulfonate. To a solution of PPh₃ (280 mg, 1.07 mmol, 4.0 eq) in THF (2 mL) was added DEAD (162 mg, 933 μmol, 3.5 eq) dropwise at 0° C. and the resulting mixture was stirred at room temperature for 0.5 h. The reaction mixture was cooled to 0° C., a solution of 2-(4-hydroxy-5-(4-hydroxy-1-methyl-1H-pyrazol-5-yl)pyrazolo[1,5-a]pyridin-2-yl)isoindoline-1,3-dione (Intermediate-26) (100 mg, 266 μmol, 1.0 eq) in DMF (0.5 mL) and a solution of (2S,3S)-3-hydroxybutan-2-yl 4-methylbenzenesulfonate (Intermediate-32) (98 mg, 400 μmol, 1.5 eq) in THF (0.5 mL) was added and the resulting mixture was stirred at 0° C. for 2 h under N₂ atmosphere. The reaction mixture was poured into water (10 mL) and extracted with EtOAc (10 mL×3). The combined organic layers were washed with brine (10 mL), dried over anhydrous Na₂SO₄, filtered and the filtrate was concentrated under reduced pressure. The residue was purified by flash silica gel chromatography (eluent of PE/EtOAc) to afford the title compound. LCMS: 602.2 [M+H]⁺.

Step 2. Synthesis of 2-((2R,3R)-2,3,7-trimethyl-2,3-dihydro-7H-pyrazolo[1,5-a]pyrazolo[3′,4′:7,8][1,4]dioxocino[5,6-c]pyridin-12-yl)isoindoline-1,3-dione. To a solution of (2S,3R)-3-((2-(1,3-dioxoisoindolin-2-yl)-5-(4-hydroxy-1-methyl-1H-pyrazol-5-yl)pyrazolo[1,5-a]pyridin-4-yl)oxy)butan-2-yl 4-methylbenzenesulfonate (120 mg, 199 μmol, 1.0 eq) in 1,4-dioxane (2 mL) was added CsF (242 mg, 1.6 mmol, 8.0 eq) and the resulting mixture was stirred at 80° C. for 1 h under N₂ atmosphere. The reaction mixture was poured into water (10 mL) and extracted with EtOAc (10 mL×3). The combined organic layers were washed with brine (10 mL), dried over anhydrous Na₂SO₄, filtered and the filtrate was concentrated under reduced pressure to afford the title compound which was used in the next step without further purification. LCMS:
430.2 [M+H]⁺.

Step 3. Synthesis of (2R,3R)-2,3,7-trimethyl-2,3-dihydro-7H-pyrazolo[1,5-a]pyrazolo[3′,4′:7,8][1,4]dioxocino[5,6-c]pyridin-12-amine. To a solution of 2-((2R,3R)-2,3,7-trimethyl-2,3-dihydro-7H-pyrazolo[1,5-a]pyrazolo[3′,4′:7,8][1,4]dioxocino[5,6-c]pyridin-12-yl)isoindoline-1,3-dione (85 mg, 198 μmol, 1.0 eq) in EtOH (1 mL) was added NH₂NH₂·H₂O (480 mg, 9.4 mmol, 98 wt %, 47.47 eq) and the resulting mixture was stirred at room temperature for 0.5 h. The reaction mixture was poured into water (10 mL) and extracted with EtOAc (10 mL×3). The combined organic layers were washed with brine (10 mL), dried over anhydrous Na₂SO₄, filtered and the filtrate was concentrated under reduced pressure. The residue was purified by flash silica gel chromatography (eluent of PE/EtOAc) to afford the title compound. LCMS: 300.1 [M+H]⁺.

### Intermediate-34: (R)-4-(2-((tert-butyldimethylsilyl)oxy)propoxy)-1-methyl-5-(4,4,5,5-tetramethyl-1,3,2-dioxaborolan-2-yl)-1H-pyrazole

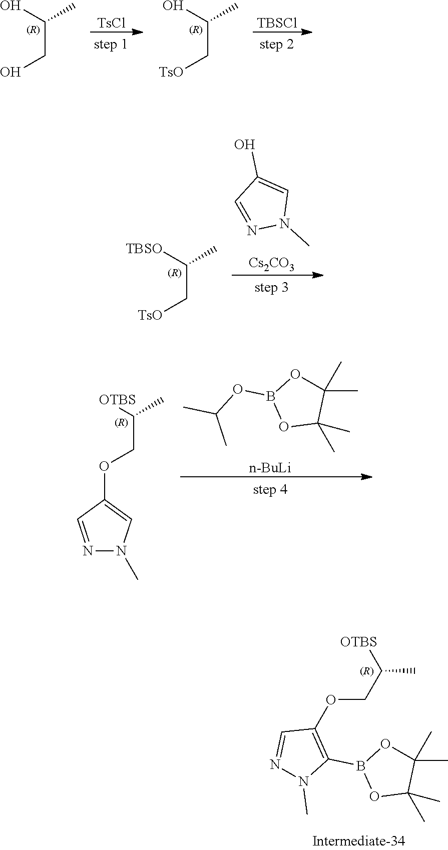

Step 1. Synthesis of (R)-2-hydroxypropyl 4-methylbenzenesulfonate. To a solution of (R)-propane-1,2-diol (10.00 g, 131.42 mmol, 1.0 eq) and TEA (17.29 g, 170.84 mmol, 1.3 eq) in DCM (150 mL) was added TsCl (22.55 g, 118.27 mmol, 0.9 eq) at 0° C., then the reaction mixture was stirred at room temperature for 1 h. The reaction mixture was poured into H₂O (200 mL), and then extracted with DCM (100 mL×2). The combined organic layers were washed with brine (30 mL), dried over anhydrous Na₂SO₄, filtered and the filtrate was concentrated under reduced pressure. The residue was purified by reverse phase HPLC (C18 column, water (0.1% FA)-ACN) to afford the title compound.

Step 2. Synthesis of (R)-2-((tert-butyldimethylsilyl)oxy)propyl 4-methylbenzenesulfonate. To a solution of (R)-2-hydroxypropyl 4-methylbenzenesulfonate (13.00 g, 56.45 mmol, 1.0 eq) in DCM (130 mL) was added imidazole (11.53 g, 169.36 mmol, 3.0 eq) and TBSCl (12.76 g, 84.68 mmol, 1.5 eq), and then the reaction mixture was stirred at room temperature for 1 h. The reaction mixture was poured into H₂O (200 mL) and then extracted with DCM (100 mL×2). The combined organic layers were washed with brine (80 mL), dried over anhydrous Na₂SO₄, filtered and the filtrate was concentrated under reduced pressure. The residue was purified by flash silica gel chromatography (eluent of EtOAc/PE) to afford the title compound. ¹H NMR (400 MHz, CDCl₃) δ=7.80 (d, 2H), 7.35 (d, 2H), 4.07-3.93 (m, 1H), 3.87-3.79 (m, 2H), 2.46 (s, 3H), 1.11 (d, 3H), 0.84 (s, 9H), 0.03 (d, 6H).

Step 3. Synthesis of (R)-4-(2-((tert-butyldimethylsilyl)oxy)propoxy)-1-methyl-1H-pyrazole. To a solution of 1-methyl-1H-pyrazol-4-ol (1.00 g, 10.19 mmol, 1.0 eq) and (R)-2-((tert-butyldimethylsilyl)oxy)propyl 4-methylbenzenesulfonate (3.16 g, 9.17 mmol, 0.9 eq) in DMF (10 mL) was added Cs₂CO₃ (9.96 g, 30.58 mmol, 3.0 eq), and then the reaction mixture was stirred at 120° C. for 3 h. The reaction mixture was filtered, the filtrate was poured into water (500 mL). The resulting solution was extracted with EtOAc (100 mL×3), the combined organic layers were washed with brine (80 mL), dried over anhydrous Na₂SO₄, filtered and concentrated under reduced pressure. The residue was purified by flash silica gel chromatography (eluent of EtOAc/PE) to afford the title compound. LCMS: 271.2 [M+H]⁺.

Step 4. Synthesis of (R)-4-(2-((tert-butyldimethylsilyl)oxy)propoxy)-1-methyl-5-(4,4,5,5-tetramethyl-1,3,2-dioxaborolan-2-yl)-1H-pyrazole. To a solution of (R)-4-(2-((tert-butyldimethylsilyl)oxy)propoxy)-1-methyl-1H-pyrazole (7.50 g, 27.73 mmol, 1.0 eq) in THF (100 mL) was added dropwise n-BuLi (2.5 M in hexane, 16.64 mL, 1.5 eq) at −60° C. under N₂ atmosphere, and the resulting mixture was stirred at −60° C. for 0.5 h after addition was complete. Then 2-isopropoxy-4,4,5,5-tetramethyl-1,3,2-dioxaborolane (7.74 g, 41.60 mmol, 1.5 eq) was added dropwise at −60° C., the resulting mixture was slowly warmed to room temperature and stirred for 0.5 h under N₂ atmosphere. The reaction mixture was slowly poured into saturated aqueous NH₄Cl (400 mL) and then extracted with EtOAc (200 mL×2). The combined organic layers were washed with brine (80 mL), dried over anhydrous Na₂SO₄, filtered and concentrated under reduced pressure. The residue was purified by flash silica gel chromatography (Eluent of EtOAc/PE) to afford the title compound. LCMS: 397.2 [M+H]⁺.

### Intermediate-35: 2-(5-(2-hydroxypropan-2-yl)pyridin-2-yl)acetic acid

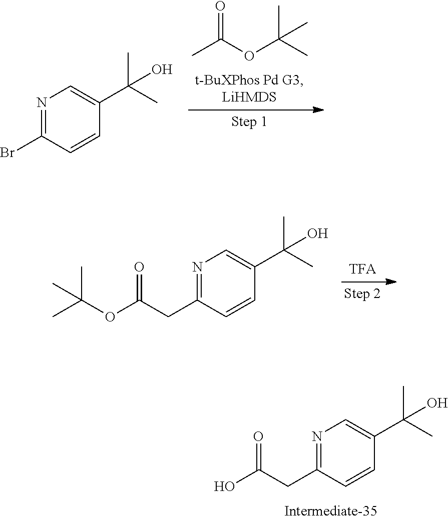

Step 1. Synthesis of tert-butyl 2-(5-(2-hydroxypropan-2-yl)pyridin-2-yl)acetate. To a solution of 2-(6-bromopyridin-3-yl)propan-2-ol (900 mg, 4.17 mmol, 1.0 eq) and tert-butyl acetate (1.2 g, 10.33 mmol, 2.48 eq) in toluene (10 mL) was added tBuXPhos Pd G3 (330.87 mg, 416.52 μmol, 0.1 eq) and LiHMDS (1 M in THF, 10.33 mL, 2.48 eq) at 0° C. under N₂ atmosphere, and the mixture was stirred at room temperature for 1.5 h under N₂ atmosphere. The reaction was quenched with saturated NH₄Cl (25 mL) and then extracted with EtOAc (25 mL×3). The combined organic layers were washed with brine (10 mL×2), dried over anhydrous Na₂SO₄, filtered and the filtrate was concentrated under reduced pressure. The residue was purified by reverse phase HPLC (C18 column, water (0.1% FA)-ACN) to afford the title compound. LCMS: 252.0 [M+H]⁺.

Step 2. Synthesis of 2-(5-(2-hydroxypropan-2-yl)pyridin-2-yl)acetic acid. To a solution of tert-butyl 2-(5-(2-hydroxypropan-2-yl)pyridin-2-yl)acetate (82 mg, 326 μmol, 1.0 eq) in DCM (0.5 mL) was added TFA (0.5 mL), and the resulting mixture was stirred at room temperature for 3 h. The reaction mixture was concentrated in vacuo to afford the title compound, which was used into the next step without further purification. LCMS: 196.1 [M+H]⁺.

### Intermediate-36: 2-(5,6-dihydro-8H-imidazo[2,1-c][1,4]oxazin-2-yl)acetic acid

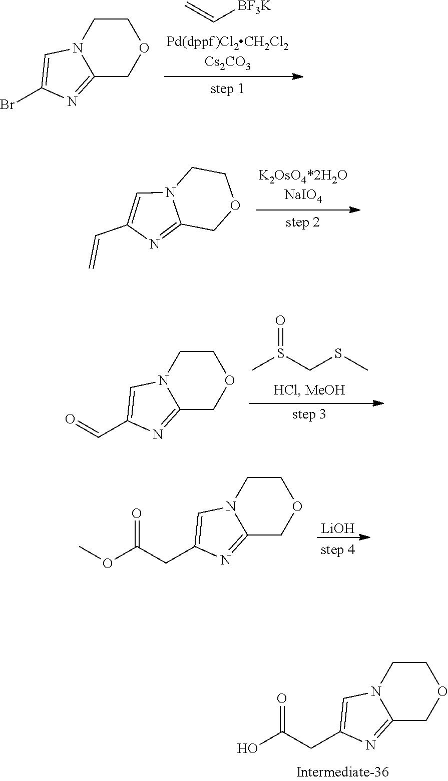

Step 1. Synthesis of 2-vinyl-5,6-dihydro-8H-imidazo[2,1-c][1,4]oxazine. To a solution of 2-bromo-5,6-dihydro-8H-imidazo[2,1-c][1,4]oxazine (1.00 g, 4.93 mmol, 1.0 eq) and trifluoro(vinyl)-14-borane, potassium salt (1.98 g, 14.78 mmol, 3.0 eq) in 1,4-dioxane (10 mL) and H₂O (2 mL) was added Pd(dppf)Cl₂*CH₂Cl₂ (804 mg, 985 μmol, 0.2 eq) and Cs₂CO₃ (3.21 g, 9.85 mmol, 2.0 eq), and the resulting mixture was stirred at 100° C. for 1.5 h under N₂ atmosphere. The reaction mixture was poured into water (40 mL) and then extracted with EtOAc (30 mL×2). The combined organic layers were washed with brine (30 mL), dried over anhydrous Na₂SO₄, filtered and the filtrate was concentrated under reduced pressure. The residue was purified by silica gel chromatography (EtOAc/PE) to afford the title compound. LCMS: 151.0 [M+H]⁺.

Step 2. Synthesis of 5,6-dihydro-8H-imidazo[2,1-c][1,4]oxazine-2-carbaldehyde. To a solution of K₂OsO₄*2H₂O (52 mg, 140 μmol, 0.035 eq) and NaIO₄ (3.42 g, 15.98 mmol, 4.0 eq) in THF (6 mL) and H₂O (6 mL) was added a solution of 2-vinyl-5,6-dihydro-8H-imidazo[2,1-c][1,4]oxazine (600 mg, 4.00 mmol, 1.0 eq) in THF (6 mL) at 0° C., then the mixture was stirred at room temperature for 1 h. The reaction mixture was poured into saturated Na₂S₂O₃ solution (100 mL) and then extracted with DCM:MeOH=10:1 (40 mL×5). The combined organic layers were washed with brine (40 mL), dried over anhydrous Na₂SO₄, filtered and the filtrate was concentrated under reduced pressure. The residue was purified by silica gel chromatography (EtOAc/PE) to afford the title compound. LCMS: 153.0 [M+H]⁺.

Step 3. Synthesis of methyl 2-(5,6-dihydro-8H-imidazo[2,1-c][1,4]oxazin-2-yl)acetate. To a solution of 5,6-dihydro-8H-imidazo[2,1-c][1,4]oxazine-2-carbaldehyde (300 mg, 1.97 mmol, 1.0 eq) and methyl((methylsulfinyl)methyl)sulfane (490 mg, 3.94 mmol, 2.0 eq) in THF (7 mL) was added N,N,N-trimethyl-1-phenylmethanaminium hydroxide (206 mg, 493 μmol, 40% in water, 0.25 eq), and the resulting mixture was stirred at 60° C. for 2 h. The reaction mixture was concentrated under reduced pressure. To the residue was added a solution of HCl in MeOH (2 M, 8 mL) and the resulting mixture was stirred at 70° C. for 1 h. The reaction mixture was concentrated under reduced pressure, then saturated aqueous NaHCO₃ (80 mL) was added and the resulting mixture was extracted with DCM:MeOH=10:1 (40 mL×5). The combined organic layers were washed with brine (40 mL), dried over anhydrous Na₂SO₄, filtered and the filtrate was concentrated under reduced pressure. The residue was purified by silica gel chromatography (EtOAc/PE) to afford the tittle compound. LCMS: 197.1 [M+H]⁺.

Step 4. Synthesis of 2-(5,6-dihydro-8H-imidazo[2,1-c][1,4]oxazin-2-yl)acetic acid. To a solution of methyl 2-(5,6-dihydro-8H-imidazo[2,1-c][1,4]oxazin-2-yl)acetate (300 mg, 1.53 mmol, 1.0 eq) in THF (2 mL), MeOH (2 mL) and H₂O (2 mL) was added LiOH*H₂O (192 mg, 4.59 mmol, 3.0 eq), and the mixture was stirred at room temperature for 0.5 h. The reaction mixture was concentrated under reduced pressure and then purified by reverse phase HPLC (C18 column, water (0.1% FA)-ACN) to afford the title compound. LCMS: 183.2 [M+H]⁺.

### Intermediate-37: 2-(5-(3-fluorobicyclo[1.1.1]pentan-1-yl)pyrimidin-2-yl)acetic acid

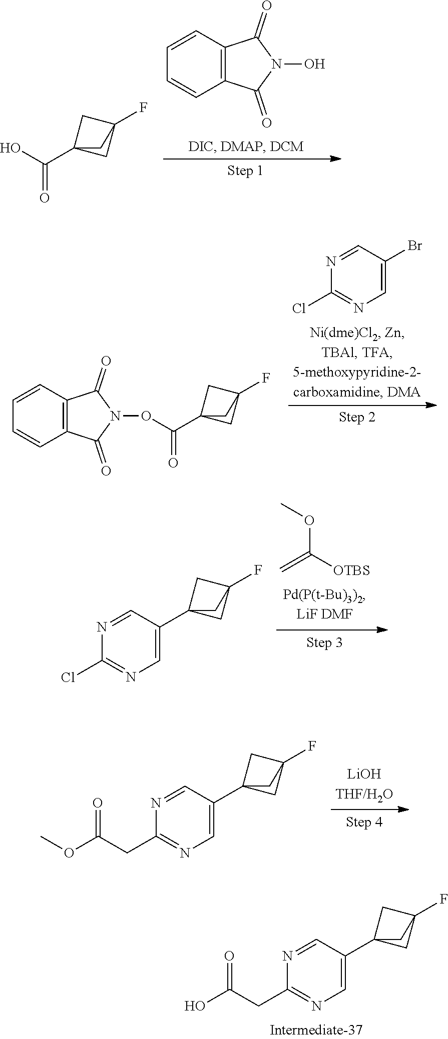

Step 1. Synthesis of 1, 3-dioxoisoindolin-2-yl 3-fluorobicyclo[1.1.1]pentane-1-carboxylate. To a solution of 3-fluorobicyclo[1.1.1]pentane-1-carboxylic acid (25.00 g, 192.14 mmol, 1 eq) and 2-hydroxyisoindoline-1,3-dione (31.34 g, 192.14 mmol, 1 eq) in DCM (250 mL) was added DIC (24.25 g, 192.14 mmol, 29.8 mL, 1 eq) and DMAP (2.35 g, 19.21 mmol, 0.1 eq) and the resulting mixture was stirred at 25° C. for 2 h. The reaction mixture was concentrated under reduced pressure to give a residue. The residue was purified by flash silica gel chromatography (eluent of PE/EtOAc) to afford the title compound. ¹H NMR (400 MHz, CDCl₃) δ=7.93-7.88 (m, 2H), 7.83-7.78 (m, 2H), 2.62 (d, 6H).

Step 2. Synthesis of 2-chloro-5-(3-fluorobicyclo[1.1.1]pentan-1-yl) pyrimidine. To a solution of 1,3-dioxoisoindolin-2-yl 3-fluorobicyclo[1.1.1]pentane-1-carboxylate (38.00 g, 138.07 mmol, 1 eq) and 5-bromo-2-chloro-pyrimidine (34.72 g, 179.49 mmol, 1.3 eq) in DMA (380 mL) was added NiCl₂(dme) (9.10 g, 41.42 mmol, 0.3 eq), 5-methoxypyridine-2-carboxamidine hydrochloride (5.18 g, 27.61 mmol, 0.2 eq) and TBAI (51.00 g, 138.07 mmol, 1 eq). Then zinc powder (36.11 g, 552.26 mmol, 4 eq) and TFA (3.15 g, 27.61 mmol, 2.05 mL, 0.2 eq) were added and the reaction mixture was degassed and purged with N₂ for 3 times and then stirred at 25° C. for 16 h under N₂ atmosphere. The resulting mixture was filtered, the filtrate was diluted with water (2000 mL) and extracted with EtOAc (1500 mL×3). The combined organic phase was washed with brine (1000 mL×2), dried with anhydrous Na₂SO₄, filtered and concentrated under reduced pressure to give a residue. The residue was purified by flash silica gel chromatography (eluent of PE/EtOAc) to afford the title compound. ¹H NMR (400 MHz, CDCl₃) δ=8.48 (s, 2H), 2.47 (d, 6H). LCMS: 199.3 [M+H]⁺.

Step 3. Synthesis of methyl 2-(5-(3-fluorobicyclo[1.1.1]pentan-1-yl) pyrimidin-2-yl) acetate. To a solution of 2-chloro-5-(3-fluorobicyclo[1.1.1]pentan-1-yl) pyrimidine (900 mg, 4.53 mmol, 1 eq) and tert-butyl-(1-methoxyvinyloxy)-dimethyl-silane (2.60 g, 13.59 mmol, 2.97 mL, 3 eq) in DMF (18 mL) was added LiF (705 mg, 27.19 mmol, 6 eq) and Pd(P(t-Bu)₃)₂ (1.20 g, 2.27 mmol, 0.5 eq) at 25° C. The reaction mixture was stirred at 80° C. for 2 h under N₂ atmosphere. The resulting mixture was cooled down to room temperature, quenched with H₂O (150 mL) and extracted with EtOAc (100 mL×3). The combined organic phase was washed with brine (100 mL×2), dried over anhydrous Na₂SO₄, filtered and the filtrate was concentrated under reduced pressure to give a residue. The residue was purified by flash silica gel chromatography (eluent of PE/EtOAc) to afford the title compound. ¹H NMR (400 MHz, CDCl₃) δ=8.56 (s, 2H), 4.04 (s, 2H), 3.75 (s, 3H), 2.45 (d, 6H). LCMS: 237.2 [M+H]⁺.

Step 4. Synthesis of 2-(5-(3-fluorobicyclo[1.1.1]pentan-1-yl)pyrimidin-2-yl)acetic acid. To a solution of methyl 2-(5-(3-fluorobicyclo[1.1.1]pentan-1-yl) pyrimidin-2-yl) acetate (300 mg, 1.27 mmol, 1 eq) in THF (3 mL) and H₂O (3 mL) was added LiOH*H₂O (160 mg, 3.81 mmol, 3 eq). The reaction mixture was stirred at 25° C. for 1 h, then concentrated under reduced pressure. The residue was purified by reverse phase preparative HPLC (C18 column, H₂O (0.225% FA)-ACN) to afford the title compound. LCMS: 223.1 [M+H]⁺.

### Intermediate-38: 2-(5-(2-methyl-1,4-dioxan-2-yl) pyridin-2-yl) acetic acid lithium salt

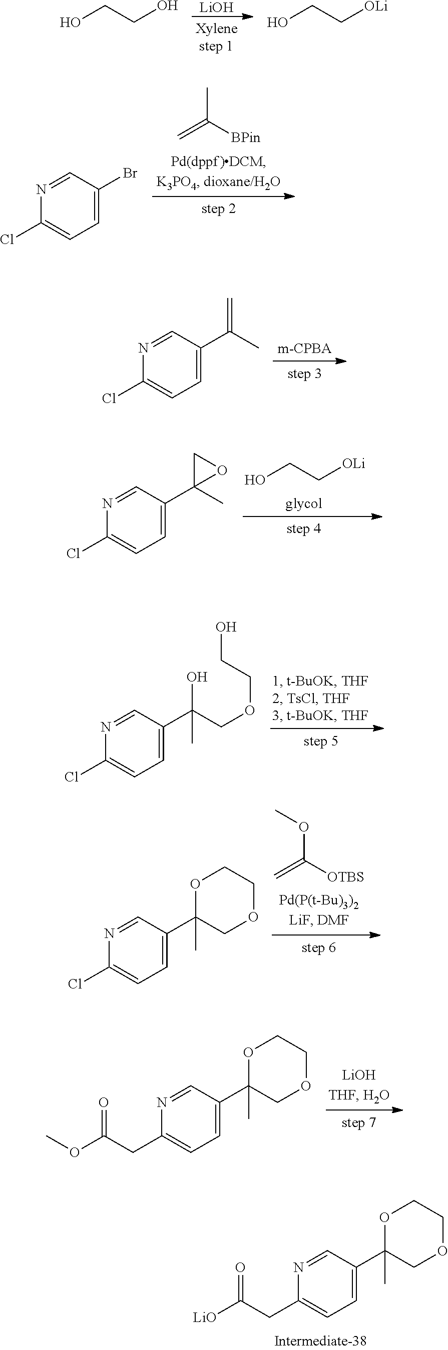

Step 1. Synthesis of lithium 2-hydroxyethan-1-olate. Glycol (14.97 g, 241.14 mmol, 13.45 mL, 1.1 eq) and LiOH (5.25 g, 219.22 mmol, 1 eq) were mixed in xylene (150 mL) and stirred at 145° C. for 5 h with Dean-Stark apparatus. The resulting mixture was filtered and the filter cake then washed with xylene (15×2 mL) and MTBE (2×30 mL) to afford the title compound which was used as such without any further purification.

Step 2. Synthesis of 2-chloro-5-(prop-1-en-2-yl) pyridine. A mixture of 5-bromo-2-chloropyridine (10.00 g, 51.96 mmol, 1 eq), 2-isopropenyl-4,4,5,5-tetramethyl-1,3,2-dioxaborolane (13.10 g, 77.95 mmol, 1.5 eq), K₃PO₄ (22.06 g, 103.93 mmol, 2 eq) and Pd(dppf)Cl₂·CH₂Cl₂ (4.24 g, 5.20 mmol, 0.1 eq) in dioxane (100 mL) and H₂O (20 mL) was degassed and purged with N₂ for 3 times, and then the mixture was stirred at 80° C. for 1 h under N₂ atmosphere. The reaction mixture was cooled down to room temperature and diluted with water (200 mL), then extracted with EtOAc (200 mL×3). The combined organic layers were washed with brine (100 mL×2), dried over anhydrous Na₂SO₄, filtered and the filtrate was concentrated under reduced pressure to give a residue. The residue was purified by flash silica gel chromatography (eluent of PE/EtOAc followed by DCM/MeOH) and reverse phase preparative HPLC (MeCN/H₂O, 0.1% FA) to afford the title compound. LCMS: 154.1 [M+H]⁺.

Step 3. Synthesis of 2-chloro-5-(2-methyloxiran-2-yl) pyridine. To a solution of 2-chloro-5-(prop-1-en-2-yl) pyridine (5.80 g, 24.54 mmol, 1 eq) in DCM (100 mL) was added m-CPBA (7.47 g, 36.81 mmol, 85% purity, 1.5 eq). The mixture was stirred at 25° C. for 3 h. The reaction mixture was quenched by saturated Na₂SO₃ aqeuous solution (100 mL). Then saturated NaHCO₃ aqeuous solution (100 mL) was added and the resulting mixture was extracted with DCM (100 mL×2). The combined organic layers were washed with brine (100 mL), dried over anhydrous Na₂SO₄, filtered and the filtrate was concentrated under reduced pressure to give a residue. The residue was purified by flash silica gel chromatography (eluent of PE/EtOAc) to afford the title compound. ¹H NMR (400 MHz, CDCl₃) δ=8.42 (d, 1H), 7.66-7.59 (m, 1H), 7.30 (d, 1H), 3.03 (d, 1H), 2.79 (d, 1H), 1.74 (s, 3H). LCMS: 170.1 [M+H]⁺.

Step 4. Synthesis of 2-(6-chloropyridin-3-yl)-1-(2-hydroxyethoxy)propan-2-ol. To a solution of 2-chloro-5-(2-methyloxiran-2-yl) pyridine (2.3 g, 13.56 mmol, 1 eq) in glycol (20 mL) was added lithium 2-hydroxyethan-1-olate (1.20 g, 17.63 mmol, 1.3 eq) (obtained as described in step 1). The reaction mixture was stirred at 120° C. for 3 h under N₂ atmosphere. The reaction mixture was concentrated under reduced pressure to give a residue. The residue was purified by flash silica gel chromatography (eluent of PE/EtOAc) to afford the title compound. ¹H NMR (400 MHz, CDCl₃) δ=8.47 (d, 1H), 7.82-7.75 (m, 1H), 7.31 (d, 1H), 3.73-3.53 (m, 6H), 1.53 (s, 3H). LCMS: 232.1 [M+H]⁺.

Step 5. Synthesis of 2-chloro-5-(2-methyl-1,4-dioxan-2-yl) pyridine. To a solution of 2-(6-chloropyridin-3-yl)-1-(2-hydroxyethoxy)propan-2-ol (2.00 g, 8.63 mmol, 1 eq) in THF (20 mL) was added t-BuOK (0.97 g, 8.63 mmol, 1 eq). The mixture was stirred at 25° C. for 1 h. Then tosyl chloride (1.65 g, 8.63 mmol, 1 eq) in THF (5 mL) was added and the reaction mixture was stirred at 25° C. for 2 h. Then another batch of t-BuOK (1.45 g, 12.95 mmol, 1.5 eq) was added and the reaction mixture was stirred at 25° C. for 1 h. The reaction mixture was diluted with water (50 mL) and extracted with EtOAc (50 mL×3). The combined organic layers were washed with brine (50 mL×2), dried over anhydrous Na₂SO₄, filtered and the filtrate was concentrated under reduced pressure to give a residue. The residue was purified by flash silica gel chromatography (eluent of PE/EtOAc) to afford the title compound. ¹H NMR (400 MHz, CDCl₃) δ=8.49 (d, 1H), 7.79-7.75 (m, 1H), 7.33 (s, 1H), 4.12-3.53 (m, 6H), 1.44 (s, 3H). LCMS: 214.1 [M+H]⁺.

Step 6. Synthesis of methyl 2-(5-(2-methyl-1,4-dioxan-2-yl) pyridin-2-yl) acetate. A mixture of 2-chloro-5-(2-methyl-1,4-dioxan-2-yl) pyridine (1.2 g, 3.76 mmol, 1 eq), tert-butyl((1-methoxyvinyl)oxy)dimethylsilane (2.13 g, 11.29 mmol, 3 eq), LiF (0.58 g, 22.58 mmol, 6 eq) and Pd(P(t-Bu)₃)₂ (0.96 g, 1.88 mmol, 0.5 eq) in DMF (12 mL) was degassed and purged with N₂ for 3 times, and then stirred at 80° C. for 3 h under N₂ atmosphere. The reaction mixture was cooled to room temperature, diluted with water (30 mL) and extracted with EtOAc (10 mL×3). The combined organic layers were washed with brine (10 mL×2), dried over anhydrous Na₂SO₄, filtered and concentrated under reduced pressure to give a residue. The residue was purified by flash silica gel chromatography (eluent of PE/EtOAc and DCM/MeOH) to afford the title compound. ¹H NMR (400 MHz, CDCl₃) δ=8.66 (d, 1H), 7.80-7.72 (m, 1H), 7.31 (d, 1H), 3.87 (s, 2H), 3.80-3.57 (m, 9H), 1.44 (s, 3H). LCMS: 252.1 [M+H]⁺.

Step 7. Synthesis of 2-(5-(2-methyl-1,4-dioxan-2-yl) pyridin-2-yl) acetic acid lithium salt. To a solution of methyl 2-(5-(2-methyl-1,4-dioxan-2-yl) pyridin-2-yl) acetate (600 mg, 1.58 mmol, 1 eq) in THF (2 mL) and H₂O (2 mL) was added LiOH*H₂O (0.13 g, 3.15 mmol, 2 eq) and the reaction mixture was stirred at 25° C. for 1 h. The reaction mixture was concentrated under reduced pressure to afford the title compound. ¹H NMR (400 MHz, D₂O) δ=8.49 (d, 1H), 7.88-7.82 (m, 1H), 7.36 (d, 1H), 4.35 (d, 1H), 3.78-3.53 (m, 7H), 1.39 (s, 3H). LCMS: 238.3 [M−Li+2H]⁺.

### Intermediate-39: 5,9-dimethyl-6,9-dihydro-5H-pyrazolo[1,5-a]pyrazolo[3′,4′:4,5]oxepino[2,3-c]pyridin-2-amine (stereoisomer 1); Intermediate-40: 5,9-dimethyl-6,9-dihydro-5H-pyrazolo[1,5-a]pyrazolo[3′,4′:4,5]oxepino[2,3-c]pyridin-2-amine (Stereoisomer 2)

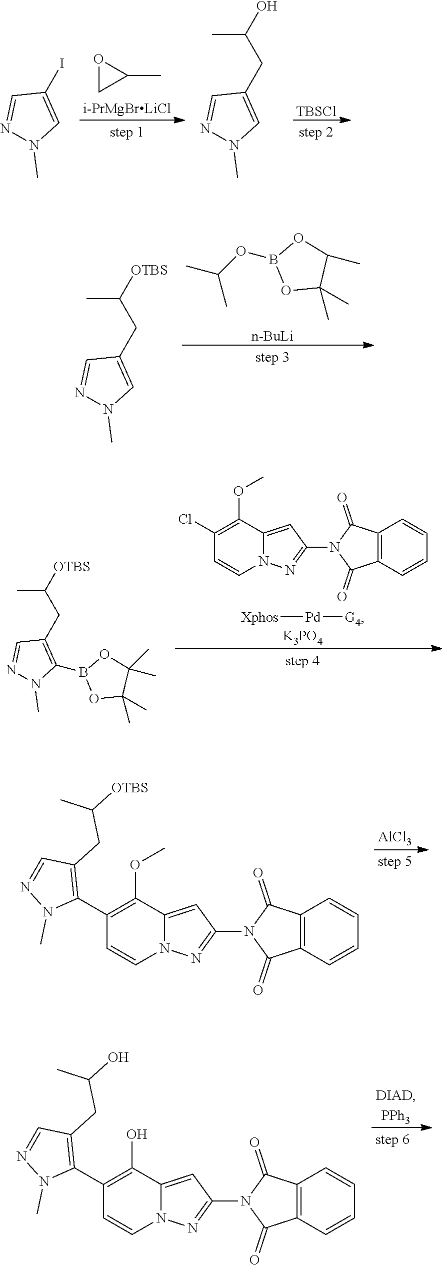

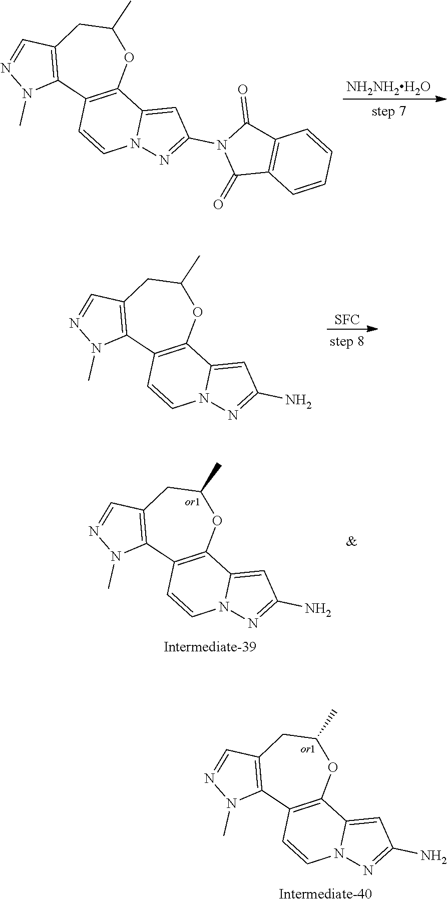 [SMILES: CC1Cc2cnn(C)c2-c2ccn3nc(N4C(=O)c5ccccc5C4=O)cc3c2O1 ; "NH2NH2·H2O" ; CC1Cc2cnn(C)c2-c2ccn3nc(N)cc3c2O1 ; "SFC" ; C[C@@H]1Cc2cnn(C)c2-c2ccn3nc(N)cc3c2O1 ; C[C@H]1Cc2cnn(C)c2-c2ccn3nc(N)cc3c2O1]

Step 1. Synthesis of 1-(1-methyl-1H-pyrazol-4-yl)propan-2-ol. To a solution of 4-iodo-1-methyl-1H-pyrazole (200.00 g, 961.54 mmol, 1.0 eq) in THF (2 L) was added i-PrMgBr*LiCl (1.3 M in THF, 887.58 mL, 1.2 eq) at 0° C., and it was stirred at 0° C. for 1 h under N₂ atmosphere. Then, the reaction mixture was cooled to −60° C., and a solution of 2-methyloxirane (72.60 g, 1.25 mol, 1.3 eq) and CuI (18.31 g, 96.15 mmol, 0.1 eq) in THF (200 mL) was added into the reaction mixture dropwise at −60° C. After addition was complete, the reaction mixture was slowly warmed to room temperature and stirred at room temperature for 0.5 h. The reaction mixture was quenched with saturated aqueous NH₄Cl (2 L) and then extracted with EtOAc (2 L×2), the combined organic layers were washed with brine (200 mL), dried over anhydrous Na₂SO₄, filtered and the filtrate was concentrated under reduced pressure. The residue was purified by flash silica gel chromatography (eluent of EtOAc/PE) to afford the title compound. LCMS: 141.2 [M+H]⁺. ¹H NMR (400 MHz, CDCl₃) δ 7.35 (s, 1H), 7.24 (s, 1H), 4.06-3.94 (m, 1H), 3.87 (s, 3H), 2.63 (d, 1H), 2.58-2.51 (m, 1H), 1.23 (d, 3H).

Step 2. Synthesis of 4-(2-((tert-butyldimethylsilyl)oxy)propyl)-1-methyl-1H-pyrazole. To a solution of 1-(1-methyl-1H-pyrazol-4-yl)propan-2-ol (170.00 g, 1.21 mol, 1.0 eq) in DCM (800 mL) was added imidazole (165.12 g, 2.43 mol, 2.0 eq) and TBSCl (201.06 g, 1.33 mol, 164.1 mL, 1.1 eq) at 0° C., and the resulting mixture was stirred at room temperature for 1 h. The reaction mixture was quenched with water (1200 mL) and extracted with DCM (800 mL×3). The combined organic layers were washed with brine (500 mL), dried over anhydrous Na₂SO₄, filtered and the filtrate was concentrated under reduced pressure. The residue was purified by flash silica gel chromatography (eluent of EtOAc/PE) to afford the title compound. LCMS: 255.2 [M+H]⁺.

Step 3. Synthesis of 4-(2-((tert-butyldimethylsilyl)oxy)propyl)-1-methyl-5-(4,4,5,5-tetramethyl-1,3,2-dioxaborolan-2-yl)-1H-pyrazole. To a solution of 4-(2-((tert-butyldimethylsilyl)oxy)propyl)-1-methyl-1H-pyrazole (64.00 g, 251.53 mmol, 1.0 eq) in THF (1 L) was added n-BuLi (2.5 M in hexane, 150.92 mL, 1.5 eq) at −60° C. under N₂ atmosphere, and the resulting mixture was stirred at −60° C. for 1 h after addition. Then 2-isopropoxy-4,4,5,5-tetramethyl-1,3,2-dioxaborolane (60.84 g, 326.99 mmol, 1.3 eq) was added at −60° C., and the reaction mixture was stirred at room temperature for 0.5 h after addition. The reaction mixture was quenched with saturated NH₄Cl (500 mL) and then extracted with EtOAc (200 mL×3), the combined organic layers were washed with brine (200 mL), dried over anhydrous Na₂SO₄, filtered and the filtrate was concentrated under reduced pressure. The residue was purified by flash silica gel chromatography (eluent of EtOAc/PE) to afford the title compound. LCMS: 380.8 [M+H]⁺.

Step 4. Synthesis of 2-(5-(4-(2-((tert-butyldimethylsilyl)oxy)propyl)-1-methyl-1H-pyrazol-5-yl)-4-methoxypyrazolo[1,5-a]pyridin-2-yl)isoindoline-1,3-dione. To a solution of 4-(2-((tert-butyldimethylsilyl)oxy)propyl)-1-methyl-5-(4,4,5,5-tetramethyl-1,3,2-dioxaborolan-2-yl)-1H-pyrazole (58.91 g, 154.86 mmol, 1.45 eq) and 2-(5-chloro-4-methoxypyrazolo[1,5-a]pyridin-2-yl)isoindoline-1,3-dione (35.00 g, 106.80 mmol, 1.0 eq) in 1,4-dioxane (550 mL) and H₂O (55 mL) was added Xphos Pd G4 (9.19 g, 10.68 mmol, 0.1 eq) and K₃PO₄ (45.34 g, 213.60 mmol, 2.0 eq), and the reaction mixture was stirred at 60° C. for 1 h under N₂ atmosphere. The reaction mixture was quenched with saturated aqueous NH₄Cl (500 mL) and extracted with EtOAc (150 mL×2). The combined organic layers were washed with brine (200 mL), dried over anhydrous Na₂SO₄, filtered and the filtrate was concentrated under reduced pressure. The residue was purified by flash silica gel chromatography (eluent of EtOAc/PE) to afford the title compound. LCMS: 546.3 [M+H]⁺.

Step 5. Synthesis of 2-(4-hydroxy-5-(4-(2-hydroxypropyl)-1-methyl-1H-pyrazol-5-yl)pyrazolo[1,5-a]pyridin-2-yl)isoindoline-1,3-dione. To a solution of 2-(5-(4-(2-((tert-butyldimethylsilyl)oxy)propyl)-1-methyl-1H-pyrazol-5-yl)-4-methoxypyrazolo[1,5-a]pyridin-2-yl)isoindoline-1,3-dione (57.40 g, 105.19 mmol, 1.0 eq) in DCE (600 mL) was added AlCl₃ (140.26 g, 1.05 mol, 10.0 eq) at room temperature and the resulting mixture was stirred at room temperature overnight. The reaction mixture was diluted with ice water (3 L) and then extracted with DCM (1.5 L×3). The combined organic layers were washed with aqueous NaHCO₃ (1 N, 350 mL×2), and brine (400 mL), dried over anhydrous Na₂SO₄, filtered and the filtrate was concentrated under reduced pressure. The residue was purified by flash silica gel chromatography (eluent of EtOAc/PE) to afford the title compound. LCMS: 418.1 [M+H]⁺.

Step 6. Synthesis of 2-(5,9-dimethyl-6,9-dihydro-5H-pyrazolo[1,5-a]pyrazolo[3′,4′:4,5]oxepino[2,3-c]pyridin-2-yl)isoindoline-1,3-dione. To a solution of PPh₃ (45.66 g, 174.07 mmol, 2.0 eq) in THF (700 mL) was added DEAD (27.28 g, 156.66 mmol, 1.8 eq) at 0° C., and the resulting mixture was stirred at room temperature for 0.5 h after addition. A solution of 2-(4-hydroxy-5-(4-(2-hydroxypropyl)-1-methyl-1H-pyrazol-5-yl)pyrazolo[1,5-a]pyridin-2-yl)isoindoline-1,3-dione (36.33 g, 87.04 mmol, 1.0 eq) in DMF (150 mL) was added into the reaction mixture dropwise at 0° C., and the resulting mixture was stirred at room temperature for 1 h. The reaction mixture was quenched with ice water (1 L) and extracted with EtOAc (800 mL×3). The combined organic layers were washed with brine (400 mL×5), dried over anhydrous Na₂SO₄, filtered and the filtrate was concentrated under reduced pressure. The residue was purified by flash silica gel chromatography (eluent of EtOAc/PE) to afford the title compound. LCMS: 400.0 [M+H]⁺.

Step 7. Synthesis of 5,9-dimethyl-6,9-dihydro-5H-pyrazolo[1,5-a]pyrazolo[3′,4′:4,5]oxepino[2,3-c]pyridin-2-amine. To a solution of 2-(5,9-dimethyl-6,9-dihydro-5H-pyrazolo[1,5-a]pyrazolo[3′,4′:4,5]oxepino[2,3-c]pyridin-2-yl)isoindoline-1,3-dione (26.00 g, 65.10 mmol, 1.0 eq) in EtOH (130 mL) was added NH₂NH₂*H₂O (69.28 g, 1.38 mol, 21.26 eq) at room temperature, and the resulting mixture was stirred at room temperature for 1 h. The reaction mixture was diluted with H₂O (500 mL) and extracted with DCM (550 mL×4). The combined organic layers were washed with brine (50 mL), dried over anhydrous Na₂SO₄, filtered and the filtrate was concentrated under reduced pressure. The residue was purified by flash silica gel chromatography (eluent of EtOAc/PE) to afford the title compound. LCMS: 270.2 [M+H]⁺.

Step 8. Separation of enantiomers of 5,9-dimethyl-6,9-dihydro-5H-pyrazolo[1,5-a]pyrazolo[3′,4′:4,5]oxepino[2,3-c]pyridin-2-amine. Racemic 5,9-dimethyl-6,9-dihydro-5H-pyrazolo[1,5-a]pyrazolo[3′,4′:4,5]oxepino[2,3-c]pyridin-2-amine (12.50 g, 46.42 mmol, 1.0 eq) was separated by chiral SFC (Instrument: Waters 350 Preparative SFC system; column: Daicel Chiralpak AS 250×50 mm I.D., 10 μm particle size; mobile phase A: CO₂, mobile phase B: MeOH (0.1% NH₃H₂O); isocratic elution: 25% phase B in supercritical CO₂; flow rate: 220 g/min; retention time: peak1—5.03 min, Peak2—5.81 min; back pressure: 100 bar; wave length: 220 nm) to afford the title compounds.

(5R)-5,9-dimethyl-6,9-dihydro-5H-pyrazolo[1,5-a]pyrazolo[3′,4′:4,5]oxepino[2,3-c]pyridin-2-amine, single isomer, first eluting isomer as stereoisomer 1 (Intermediate-39), retention time: 5.03 minutes. LCMS: 270.2 [M+H]⁺.

(5R)-5,9-dimethyl-6,9-dihydro-5H-pyrazolo[1,5-a]pyrazolo[3′,4′:4,5]oxepino[2,3-c]pyridin-2-amine, single isomer, second eluting isomer as stereoisomer 2 (Intermediate-40), retention time: 5.81 minutes. LCMS: 270.2 [M+H]⁺.

### Intermediate-41: 10-methyl-5,6-dihydro-10H-pyrazolo[3″,4″:7′,8′][1,4]dioxocino[5′,6′:3,4]-benzo[1,2-d]thiazol-2-amine

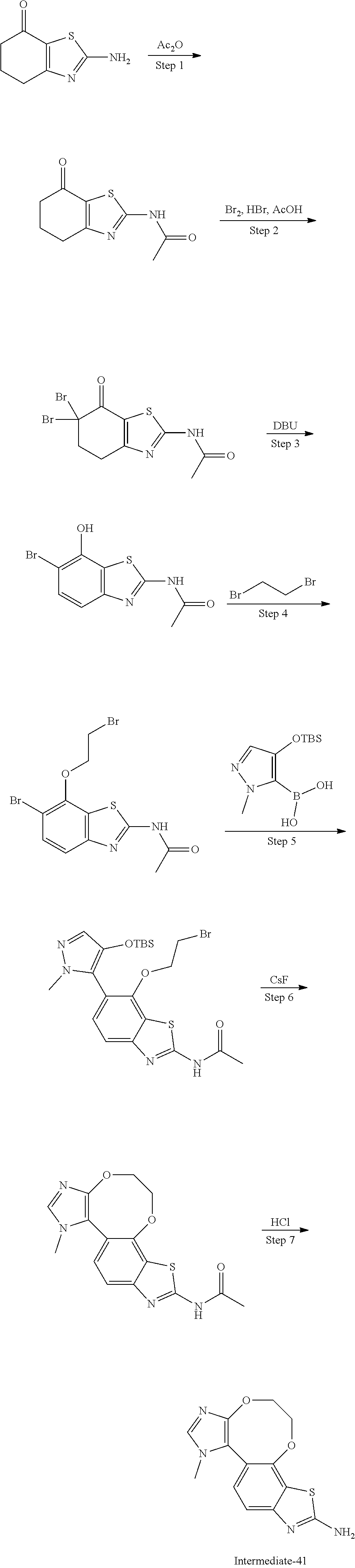

Step 1. Synthesis of N-(7-oxo-4,5,6,7-tetrahydrobenzo[d]thiazol-2-yl)acetamide. 2-Amino-5,6-dihydrobenzo[d]thiazol-7(4H)-one (11.00 g, 65.39 mmol, 1.0 eq) was added into Ac₂O (50 mL), and the reaction mixture was stirred at 140° C. for 2 h. The resulting mixture was cooled down to room temperature, MTBE (100 mL) was added and the resulting mixture was filtered. The filter cake was washed with MTBE (100 mL×2) and then dried under reduced pressure to afford the title compound, which was used in the next step without further purification. LCMS: 210.9 [M+H]⁺.

Step 2. Synthesis of N-(6,6-dibromo-7-oxo-4,5,6,7-tetrahydrobenzo[d]thiazol-2-yl)acetamide. To a suspension of N-(7-oxo-4,5,6,7-tetrahydrobenzo[d]thiazol-2-yl)acetamide (5.00 g, 23.78 mmol, 1.0 eq) in AcOH (50 mL) was added HBr (801 mg, 4.76 mmol, 48% in AcOH, 0.2 eq) and Br₂ (15.20 g, 95.12 mmol, 4.90 mL, 4.0 eq), and the resulting mixture was stirred at 60° C. overnight. The reaction mixture was diluted with H₂O (200 mL), the resulting suspension was filtered and the filtered cake was dried under reduced pressure to afford the title compound, which was used into the next step without further purification. LCMS: 368.8 [M+H]⁺.

Step 3. Synthesis of N-(6-bromo-7-hydroxybenzo[d]thiazol-2-yl)acetamide. To a solution of N-(6,6-dibromo-7-oxo-4,5,6,7-tetrahydrobenzo[d]thiazol-2-yl)acetamide (6.00 g, 16.30 mmol, 1.0 eq) in THF (60 mL) was added DBU (7.45 g, 48.91 mmol, 3.0 eq), and the mixture was stirred at room temperature for 1 h. The reaction mixture was concentrated under reduced pressure, the residue was suspended in H₂O (50 mL) and saturated aqueous NH₄Cl (20 mL), the resulting suspension was filtered and the filtered cake was dried under reduced pressure to afford the title compound, which was used into the next step without further purification. LCMS: 286.8 [M+H]⁺.

Step 4. Synthesis of N-(6-bromo-7-(2-bromoethoxy)benzo[d]thiazol-2-yl)acetamide. To a solution of N-(6-bromo-7-hydroxybenzo[d]thiazol-2-yl)acetamide (1.00 g, 3.48 mmol, 1.0 eq) in DMF (10 mL) was added K₂CO₃ (481 mg, 3.48 mmol, 1.0 eq) and 1,2-dibromoethane (3.27 g, 17.41 mmol, 5.0 eq), and the mixture was stirred at room temperature for 3 h. The reaction mixture was quenched with H₂O (100 mL) and extracted with EtOAc (50 mL×3). The combined organic layers were washed with brine (30 mL), dried over anhydrous Na₂SO₄, filtered and concentrated under reduced pressure. The residue was purified by silica gel chromatography (EtOAc/PE) to afford the tittle compound. LCMS: 394.7 [M+H]⁺.

Step 5. Synthesis of N-(7-(2-bromoethoxy)-6-(4-((tert-butyldimethylsilyl)oxy)-1-methyl-1H-pyrazol-5-yl)benzo[d]thiazol-2-yl)acetamide. To a solution of N-(6-bromo-7-(2-bromoethoxy) benzo[d]thiazol-2-yl) acetamide (750 mg, 1.90 mmol, 1.0 eq) and (4-((tert-butyldimethylsilyl)oxy)-1-methyl-1H-pyrazol-5-yl)boronic acid (731 mg, 2.85 mmol, 1.5 eq) in 1,4-dioxane (10 mL) and H₂O (1 mL) was added Xphos Pd G4 (328 mg, 381 μmol, 0.2 eq) and K₃PO₄ (808 mg, 3.81 mmol, 2 eq), and the resulting mixture was stirred at 60° C. for 1 h under N₂ atmosphere. The reaction mixture was quenched with H₂O (50 mL) and extracted with EtOAc (50 mL×3). The combined organic layers were washed with brine (30 mL), dried over anhydrous Na₂SO₄, filtered and the filtrate was concentrated under reduced pressure. The residue was purified by silica gel chromatography (EtOAc/PE) to afford the tittle compound. LCMS: 524.9 [M+H]⁺.

Step 6. Synthesis of N-(10-methyl-5,6-dihydro-10H-pyrazolo[3″,4″:7′,8′][1,4]dioxocino[5′,6′:3,4]benzo[1,2-d]thiazol-2-yl)acetamide. To a mixture of N-(7-(2-bromoethoxy)-6-(4-((tert-butyldimethylsilyl)oxy)-1-methyl-1H-pyrazol-5-yl)benzo[d]thiazol-2-yl)acetamide (760 mg, 1.45 mmol, 1.0 eq) in DMF (10 mL) was added CsF (659 mg, 4.34 mmol, 3.0 eq), and it the resulting mixture was stirred at 40° C. for 1 h. The reaction mixture was quenched with H₂O (30 mL) and extracted with EtOAc (30 mL×3). The combined organic layers were washed with brine (30 mL), dried over anhydrous Na₂SO₄, filtered and the filtrate was concentrated under reduced pressure to afford the title compound, which was used into the next step without further purification. LCMS: 331.0 [M+H]⁺.

Step 7. Synthesis of 10-methyl-5,6-dihydro-10H-pyrazolo[3″,4″:7′,8′][1,4]dioxocino[5′,6′:3,4]benzo[1,2-d]thiazol-2-amine. To a solution of HCl in MeOH (2 M, 10 mL) was added N-(10-methyl-5,6-dihydro-10H-pyrazolo[3″,4″:7′,8′][1,4]dioxocino[5′,6′:3,4]benzo[1,2-d]thiazol-2-yl)acetamide (480 mg, 1.16 mmol, 1.0 eq) and H₂O (2.5 mL), and the resulting mixture was stirred at 60° C. for 1 h. The reaction mixture concentrated under reduced pressure and then purified by reverse phase HPLC (C18 column, water (0.1% FA)-ACN) to afford the title compound. LCMS: 289.0 [M+H]⁺.

### Intermediate-42: rac-4-(((2R,3R)-3-((tert-butyldimethylsilyl)oxy)butan-2-yl)oxy)-1-methyl-5-(4,4,5,5-tetramethyl-1,3,2-dioxaborolan-2-yl)-1H-pyrazole

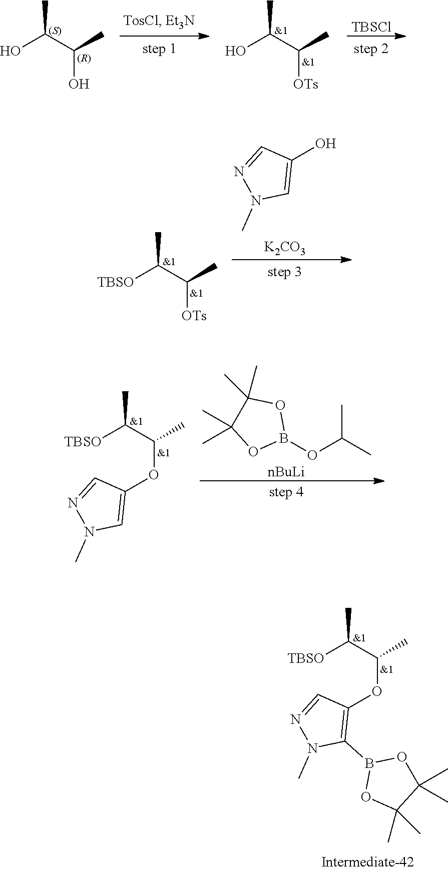

Step 1. Synthesis rac-(2R,3S)-3-hydroxybutan-2-yl 4-methylbenzenesulfonate. To a solution of the (2R, 3S)-2,3-butanediol (50.00 g, 554.81 mmol, 1.0 eq) and TEA (84.21 g, 832.22 mmol, 1.5 eq) in DCM (500 mL) was added TsCl (95.20 g, 499.33 mmol, 0.9 eq) at 0° C., and the resulting mixture was stirred at 40° C. overnight. The reaction mixture was poured into H₂O (1000 mL) and extracted with DCM (300 mL×2). The combined organic layers were dried over anhydrous Na₂SO₄, filtered and the filtrate was concentrated under reduced pressure to give a residue. The residue was purified by reverse phase HPLC (water (0.1% FA)-ACN) to afford the title compound. LCMS: 267.1 [M+Na]⁺.

Step 2. Synthesis of rac-(2R,3S)-3-((tert-butyldimethylsilyl)oxy)butan-2-yl 4-methylbenzenesulfonate. To a solution of rac-(2R,3S)-3-hydroxybutan-2-yl 4-methylbenzenesulfonate (24.00 g, 98.24 mmol, 1.0 eq) in DCM (240 mL) was added imidazole (13.38 g, 196.47 mmol, 2.0 eq) and TBSCl (19.25 g, 127.71 mmol, 1.3 eq) at 0° C., and the resulting mixture was stirred at 30° C. for 3 h. The reaction mixture was quenched with H₂O (600 mL) and extracted with DCM (300 mL×3). The combined organic layers were dried over anhydrous Na₂SO₄, filtered and the filtrate was concentrated under reduced pressure. The residue was purified by silica gel chromatography (EtOAc/PE) to afford the title compound. LCMS: 359.2 [M+H]⁺.

¹H NMR (400 MHz, CDCl₃) δ 7.83 (d, 2H), 7.36 (d, 2H), 4.49-4.40 (m, 1H), 3.95-3.86 (m, 1H), 2.48 (s, 3H), 1.24 (d, 3H), 1.07 (d, 3H), 0.88 (s, 9H), 0.06 (s, 6H).

Step 3. Synthesis of the rac-4-(((2R,3R)-3-((tert-butyldimethylsilyl)oxy)butan-2-yl)oxy)-1-methyl-1H-pyrazole. To a solution of 1-methylpyrazol-4-ol (9 g, 91.74 mmol, 1.0 eq) and rac-(2R,3S)-3-((tert-butyldimethylsilyl)oxy)butan-2-yl 4-methylbenzenesulfonate (32.90 g, 91.74 mmol, 1.0 eq) in DMA (90 mL) was added Cs₂CO₃ (89.67 g, 275.22 mmol, 3.0 eq), and the resulting mixture was stirred at 120° C. under N₂ for 1 h. The reaction mixture was diluted with H₂O (800 mL), and then extracted with EtOAc (500 mL×3). The combined organic layers were washed with brine (600 mL), dried over anhydrous Na₂SO₄, filtered and the filtrate was concentrated under reduced pressure. The residue was purified by flash silica gel chromatography (Eluent of EtOAc/PE) to afford the title compound. LCMS: 285.2 [M+H]⁺.

Step 4. Synthesis of rac-4-(((2R,3R)-3-((tert-butyldimethylsilyl)oxy)butan-2-yl)oxy)-1-methyl-5-(4,4,5,5-tetramethyl-1,3,2-dioxaborolan-2-yl)-1H-pyrazole. To a solution of the rac-4-(((2R,3R)-3-((tert-butyldimethylsilyl)oxy)butan-2-yl)oxy)-1-methyl-1H-pyrazole (6.5 g, 22.85 mmol, 1.0 eq) in THF (70 mL) was added dropwise n-BuLi (2.5 M in THF, 13.71 mL, 1.5 eq) at −60° C. under N₂ atmosphere and the reaction mixture was stirred at −60° C. for 0.5 h under N₂. Then 2-isopropoxy-4, 4, 5, 5-tetramethyl-1, 3, 2-dioxaborolane (5.53 g, 29.70 mmol, 1.3 eq) was added at −60° C., and the resulting mixture was stirred at room temperature for 0.5 hr under N₂. The resulting mixture was quenched with H₂O (90 mL) and extracted with EtOAc (90 mL×3), the combined organic layers were washed with brine (10 mL), dried over anhydrous Na₂SO₄, filtered and the filtrate was concentrated under reduced pressure to give a residue. The residue was purified by flash silica gel chromatography (Eluent of EtOAc/PE) to afford the title compound. LCMS: 411.4 [M+H]⁺.

### Intermediate-43: (1S,2R)—N-(6-bromo-5-chloroimidazo[1,2-a]pyridin-2-yl)-2-(5-methylpyrimidin-2-yl)cyclopropane-1-carboxamide

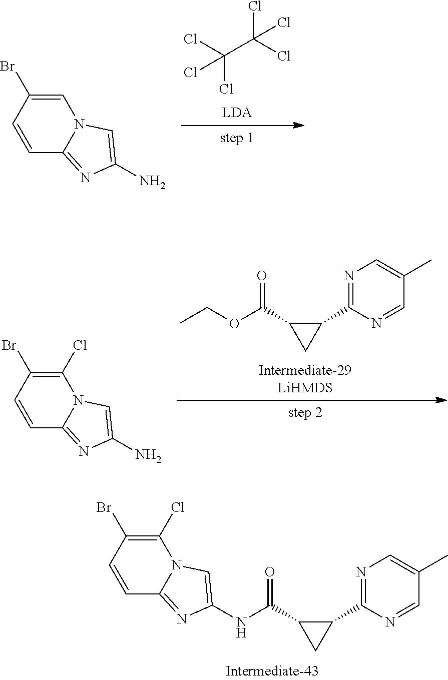 [SMILES: Nc1cn2cc(Br)ccc2n1 ; ClC(Cl)(Cl)C(Cl)(Cl)Cl ; Nc1cn2c(Cl)c(Br)ccc2n1 ; CCOC(=O)[C@H]1C[C@H]1c1ncc(C)cn1 ; Cc1cnc([C@@H]2C[C@@H]2C(=O)Nc2cn3c(Cl)c(Br)ccc3n2)nc1]

Step 1. Synthesis of 6-bromo-5-chloro-imidazo[1, 2-a]pyridin-2-amine. To a solution of 6-bromoimidazo[1, 2-a]pyridin-2-amine (15.00 g, 70.74 mmol, 1.0 eq) in THF (150 mL) was added LDA (2 M, 124 mL, 3.5 eq) at −65° C. under N₂, the mixture was stirred at −65° C. for 0.5 h. Then 1, 1, 1, 2, 2, 2-hexachloroethane (16.75 g, 70.74 mmol, 8 mL, 1.0 eq) in THF (100 mL) was added dropwise to the mixture at −65° C. under N₂. The resulting mixture was stirred at −65° C. for 0.5 h. The reaction mixture was poured into saturated NH₄Cl aqueous solution (150 mL), and then extracted with EtOAc (100 mL×3). The combined organic layers were washed with brine (100 mL), dried over anhydrous Na₂SO₄, filtered and the filtrate was concentrated under reduced pressure to give a residue. The residue was purified by flash silica gel chromatography (eluent of EtOAc/PE) to afford the title compound. LCMS: 248.0 [M+H]⁺.

Step 2. Synthesis of (1S, 2R)—N-(6-bromo-5-chloro-imidazo[1, 2-a]pyridin-2-yl)-2-(5-methylpyrimidin-2-yl) cyclopropanecarboxamide. To a solution of 6-bromo-5-chloro-imidazo[1, 2-a]pyridin-2-amine (7.00 g, 28.40 mmol, 1.0 eq) and ethyl (1S, 2R)-2-(5-methylpyrimidin-2-yl) cyclopropanecarboxylate (7.61 g, 36.92 mmol, 1.3 eq) in THF (70 mL) was added dropwise LiHMDS (1 M in THF, 71 mL, 2.5 eq) at 0° C. under N₂. The mixture was stirred at 40° C. under N₂ for 1 h. The reaction mixture was poured into saturated NH₄Cl aqueous solution (150 mL), and then extracted with EtOAc (150 mL×3). The combined organic layers were washed with brine (200 mL), dried over anhydrous Na₂SO₄, filtered and the filtrate was concentrated under reduced pressure to give a residue. The residue was purified by flash silica gel chromatography (eluent of EtOAc/PE) to afford the title compound. LCMS: 408.0 [M+H]⁺.

### Intermediate-44: rel-(1R)-1-(5-methylpyrimidin-2-yl)ethanol, single isomer, first eluting isomer as isomer 1; Intermediate-45: rel-(1R)-1-(5-methylpyrimidin-2-yl)ethanol, single isomer, second eluting isomer as isomer 2

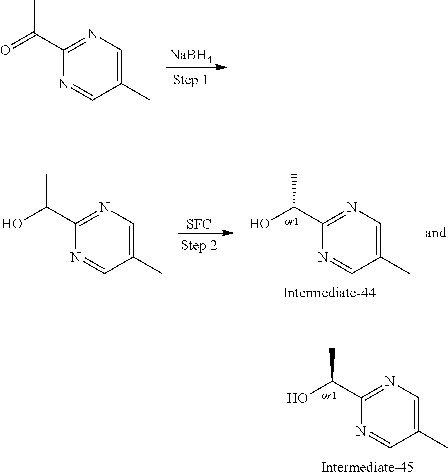

Step 1. Synthesis of racemic 1-(5-methylpyrimidin-2-yl)ethan-1-ol. To a solution of 1-(5-methylpyrimidin-2-yl)ethan-1-one (2.20 g, 16.16 mmol, 1.0 eq) in EtOH (20 mL) was added NaBH₄ (970 mg, 25.64 mmol, 1.59 eq) slowly at 0° C., and the resulting mixture was stirred at 0° C. for 0.1 h. The reaction mixture was quenched with water (100 mL), extracted with EtOAc (100 mL×3). The combined organic layers were washed with brine (50 mL), dried over anhydrous Na₂SO₄, filtered and concentrated under reduced pressure. The residue was purified by flash silica gel chromatography (Eluent of EtOAc/PE) to afford the title compound. ¹H NMR (400 MHz, CDCl₃) δ 8.55 (s, 2H), 4.92 (br d, 1H), 4.17 (br d, 1H), 2.33 (s, 3H), 1.55 (d, 3H).

Step 2. Chiral separation of enantiomers of 1-(5-methylpyrimidin-2-yl)ethanol. Racemic 1-(5-methylpyrimidin-2-yl)ethan-1-ol (200 mg, 2.13 mmol, 1.0 eq) was separated by chiral SFC (Instrument: Waters 80 Preparative SFC system; column: daicel chiralpak IC 250×30 mm I.D., 10 μm particle size; mobile phase A: CO₂, mobile phase B: IPA (0.1% NH₃H₂O); isocratic elution: 35% phase B in supercritical CO₂; flow rate: 65 g/min; retention time: peak1/5.35 min, peak2/7.78 min; back pressure: 100 bar; wave length: 220 nm) to afford the tittle compounds. Rel-(1R)-1-(5-methylpyrimidin-2-yl)ethanol, single isomer, first eluting isomer as isomer 1, retention time: 5.35 minutes. (Intermediate-44); Rel-(1R)-1-(5-methylpyrimidin-2-yl)ethanol, single isomer, second eluting isomer as isomer 2, retention time: 7.78 minutes (Intermediate-45).

### Intermediate-46: 4-nitrophenyl ((2R,3R)-2,3,7-trimethyl-2,3-dihydro-7H-pyrazolo[1,5-a]pyrazolo[3′,4′:7,8][1,4]dioxocino[5,6-c]pyridin-12-yl)carbamate

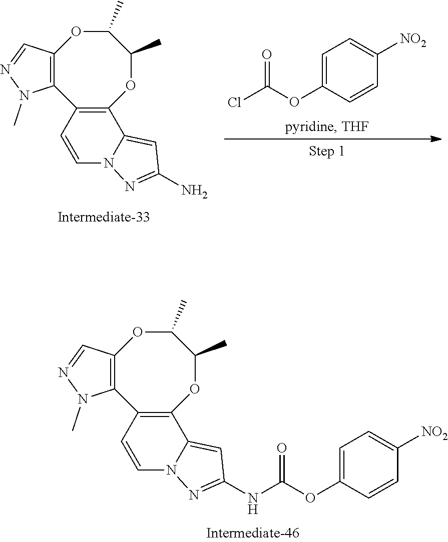 [SMILES: C[C@H]1Oc2cnn(C)c2-c2ccn3nc(N)cc3c2O[C@@H]1C ; O=C(Cl)Oc1ccc([N+](=O)[O-])cc1 ; C[C@H]1Oc2cnn(C)c2-c2ccn3nc(NC(=O)Oc4ccc([N+](=O)[O-])cc4)cc3c2O[C@@H]1C]

Step 1. To a solution of (2R,3R)-2,3,7-trimethyl-2,3-dihydro-7H-pyrazolo[1,5-a]pyrazolo[3′,4′:7,8][1,4]dioxocino[5,6-c]pyridin-12-amine (Intermediate-33) (100 mg, 334 μmol, 1.0 eq) and pyridine (24 mg, 301 μmol, 1.5 eq) in THF (1 mL) was added 4-nitrophenyl carbonochloridate (81 mg, 401 μmol, 1.2 eq) at 0° C., and the reaction mixture was stirred at room temperature for 0.5 h. The reaction mixture was concentrated under reduced pressure to afford the title compound, which was used into the next step without further purification. LCMS: 465.0 [M+H]⁺.

### Intermediate-47: 3-(1-(benzyloxy)ethyl)-5-(3-((tert-butyldimethylsilyl)oxy)propyl)-4-(4,4,5,5-tetramethyl-1,3,2-dioxaborolan-2-yl)isoxazole

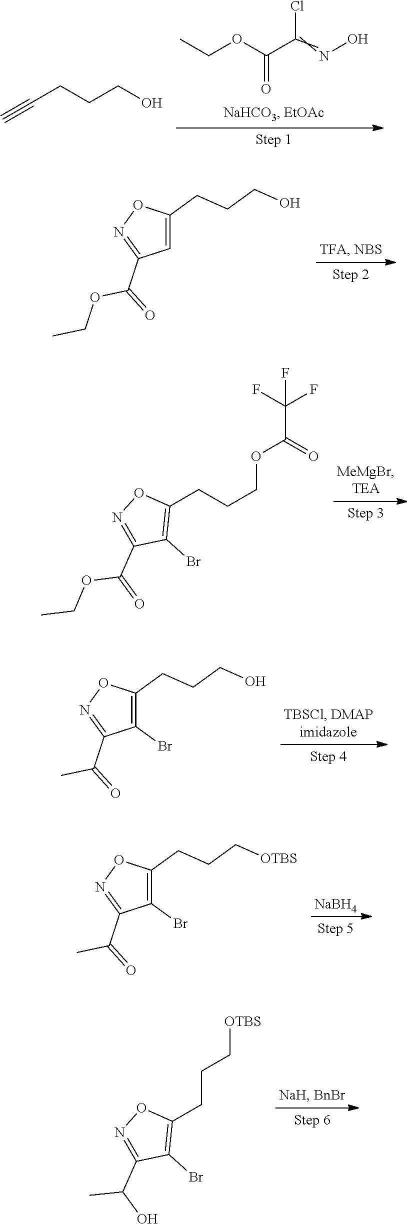

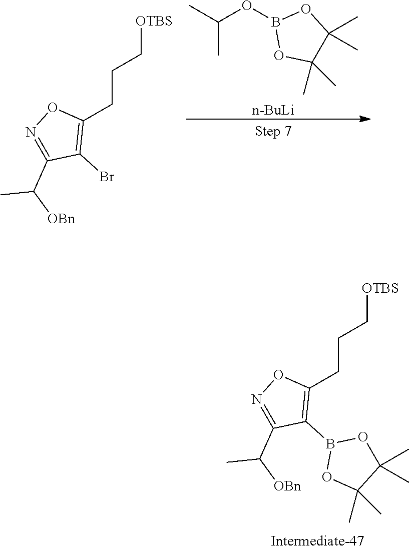

Step 1. Synthesis of ethyl 5-(3-hydroxypropyl)isoxazole-3-carboxylate. To a solution of pent-4-yn-1-ol (10.00 g, 118.88 mmol, 1.0 eq) and ethyl 2-chloro-2-(hydroxyimino)acetate (54.05 g, 356.65 mmol, 3.0 eq) in EtOAc (100 mL) was added NaHCO₃ (29.96 g, 356.65 mmol, 3.0 eq), and the resulting mixture was stirred at 90° C. for 2 h. The reaction mixture was filtered and the filtrate was concentrated under reduced pressure. The residue was purified by flash silica gel chromatography (Eluent of EtOAc/PE) to afford the title compound. LCMS: 200.2 [M+H]⁺.

Step 2. Synthesis of ethyl 4-bromo-5-(3-(2,2,2-trifluoroacetoxy)propyl)isoxazole-3-carboxylate. To a solution of ethyl 5-(3-hydroxypropyl)isoxazole-3-carboxylate (20.00 g, 100.40 mmol, 1.0 eq) in TFA (100 mL) was added NBS (21.44 g, 120.48 mmol, 1.2 eq) at 0° C., and the resulting mixture was stirred at 120° C. for 2 h. The reaction mixture was basified with saturated aqueous NaHCO₃ solution to pH=8 and then extracted with EtOAc (500 mL×3). The combined organic layers were washed with brine (200 mL), dried over anhydrous Na₂SO₄, filtered and the filtrate was concentrated under reduced pressure. The residue was purified by flash silica gel chromatography (eluent of EtOAc/PE) to afford the title compound. LCMS: 376.1 [M+H]⁺.

Step 3. Synthesis of 1-(4-bromo-5-(3-hydroxypropyl)isoxazol-3-yl)ethan-1-one. To a solution of ethyl 4-bromo-5-(3-(2,2,2-trifluoroacetoxy)propyl)isoxazole-3-carboxylate (9.00 g, 24.06 mmol, 1.0 eq) in toluene (500 mL) was added TEA (14.12 g, 139.53 mmol, 5.8 eq) at 0° C. under N₂ atmosphere, followed by methylmagnesium bromide (3 M in THF, 16.04 mL, 2.0 eq), and the resulting mixture was stirred at 0° C. for 2 h under N₂ atmosphere. The reaction mixture was quenched with ice water (500 mL), the resulting suspension was filtered. The filtrate was extracted with EtOAc (200 mL×3). The combined organic layers were washed with brine (100 mL), dried over anhydrous Na₂SO₄, filtered and the filtrate was concentrated under reduced pressure. The residue was purified by flash silica gel chromatography (eluent of EtOAc/PE) to afford the title compound. LCMS: 249.9 [M+H]⁺.

Step 4. Synthesis of 1-(4-bromo-5-(3-((tert-butyldimethylsilyl)oxy)propyl)isoxazol-3-yl)ethan-1-one. To a solution of 1-(4-bromo-5-(3-hydroxypropyl)isoxazol-3-yl)ethan-1-one (8.00 g, 32.25 mmol, 1.0 eq), N,N-dimethylpyridin-4-amine (118 mg, 967 μmol, 0.03 eq) and imidazole (2.41 g, 35.47 mmol, 1.1 eq) in DCM (160 mL) was added TBSCl (5.83 g, 38.70 mmol, 1.2 eq) at 0° C., and the resulting mixture was stirred at room temperature for 2 h. The reaction mixture was quenched with water (200 mL) and extracted EtOAc (300 mL×2). The combined organic layers were washed with brine (200 mL), dried over anhydrous Na₂SO₄, filtered and the filtrate was concentrated under reduced pressure. The residue was purified by flash silica gel chromatography (eluent of EtOAc/PE) to afford the title compound. LCMS: 364.1 [M+H]⁺.

Step 5. Synthesis of 1-(4-bromo-5-(3-((tert-butyldimethylsilyl)oxy)propyl)isoxazol-3-yl)ethan-1-ol. To a solution of 1-(4-bromo-5-(3-((tert-butyldimethylsilyl)oxy)propyl)isoxazol-3-yl)ethan-1-one (4.9 g, 13.52 mmol, 1.0 eq) in THF (40 mL) was added NaBH₄ (256 mg, 6.76 mmol, 0.5 eq) at 0° C., and the resulting mixture was stirred at room temperature for 0.5 h. The reaction mixture was quenched with saturated aqueous NH₄Cl (200 mL), extracted with EtOAc (200 mL×2), the combined organic layers were washed with brine (10 mL), dried with anhydrous Na₂SO₄, filtered and the filtrate was concentrated under reduced pressure to afford the title compound, which was used into the next step without further purification. LCMS: 366.2 [M+H]⁺.

Step 6. Synthesis of 3-(1-(benzyloxy)ethyl)-4-bromo-5-(3-((tert-butyldimethylsilyl)oxy)propyl)isoxazole. To a solution of 1-(4-bromo-5-(3-((tert-butyldimethylsilyl)oxy)propyl)isoxazol-3-yl)ethan-1-ol (4.90 g, 13.45 mmol, 1.0 eq) in THF (50 mL) was added NaH (1.08 g, 26.90 mmol, 60% in mineral oil, 2.0 eq) at 0° C., and the mixture was stirred at 0° C. for 0.5 h. Then, BnBr (2.76 g, 16.14 mmol, 1.2 eq) was added into the mixture at 0° C., and the reaction mixture was stirred at room temperature overnight under N₂ atmosphere. The reaction mixture was quenched with saturated aqueous NH₄Cl (50 mL) and extracted with EtOAc (50 mL×3). The combined organic layers were washed with brine (30 mL), dried over anhydrous Na₂SO₄, filtered and the filtrate was concentrated under reduced pressure. The residue was purified by flash silica gel chromatography (eluent of EtOAc/PE) to afford the title compound. LCMS: 454.0 [M+H]⁺.

Step 7. Synthesis of 3-(1-(benzyloxy)ethyl)-5-(3-((tert-butyldimethylsilyl)oxy)propyl)-4-(4,4,5,5-tetramethyl-1,3,2-dioxaborolan-2-yl)isoxazole. To a solution of 3-(1-(benzyloxy)ethyl)-4-bromo-5-(3-((tert-butyldimethylsilyl)oxy)propyl)isoxazole (2.00 g, 4.40 mmol, 1.0 eq) in THF (20 mL) was added n-BuLi (2.5 M in THF, 2.11 mL, 1.2 eq) at −65° C., and the resulting mixture was stirred at −65° C. for 0.5 h. 2-Isopropoxy-4,4,5,5-tetramethyl-1,3,2-dioxaborolane (983 mg, 5.28 mmol, 1.2 eq) was added into the reaction mixture at −65° C., and then the resulting mixture was slowly warmed up to room temperature and stirred for 1 h under N₂ atmosphere. The reaction mixture was quenched with water (30 mL) and extracted with EtOAc (30 mL×3). The combined organic layers were washed with brine (20 mL), dried over anhydrous Na₂SO₄, filtered and the filtrate was concentrated under reduced pressure. The residue was purified by flash silica gel chromatography (eluent of EtOAc/PE) to afford the title compound. LCMS: 502.3 [M+H]⁺.

### Intermediate-48: 2-(5-chloro-4-(methoxymethoxy)pyrazolo[1,5-a]pyridin-2-yl)isoindoline-1,3-dione

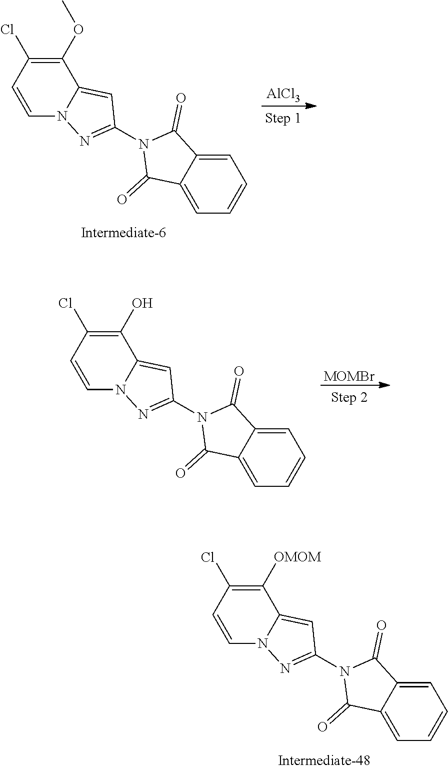

Step 1. Synthesis of 2-(5-chloro-4-hydroxypyrazolo[1,5-a]pyridin-2-yl)isoindoline-1,3-dione. To a solution of 2-(5-chloro-4-methoxypyrazolo[1,5-a]pyridin-2-yl)isoindoline-1,3-dione (Intermediate-6) (25.00 g, 76.28 mmol, 1.0 eq) in DCE (1000 mL) was added AlCl₃ (101.72 g, 762.84 mmol, 10.0 eq) portionwise at room temperature, and then it was stirred at room temperature for 20 min. The reaction mixture was slowly poured into aqueous HCl solution (2 N, 2 L), the resulting suspension was filtered and the filter cake was dried under reduced pressure to afford the title compound, which was used in the next step without further purification. LCMS: 314.1 [M+H]⁺.

Step 2. Synthesis of 2-(5-chloro-4-(methoxymethoxy)pyrazolo[1,5-a]pyridin-2-yl)isoindoline-1,3-dione. To a solution of 2-(5-chloro-4-hydroxypyrazolo[1,5-a]pyridin-2-yl)isoindoline-1,3-dione (1.00 g, 3.19 mmol, 1.0 eq) in DMA (10 mL) was added DIEA (824 mg, 6.38 mmol, 2.0 eq) and MOMBr (478 mg, 3.83 mmol, 1.2 eq) at 0° C., and the resulting mixture was stirred at room temperature for 1 h under N₂ atmosphere. The reaction mixture was quenched with H₂O (50 mL), the resulting suspension was filtered, the filter cake was dried under reduced pressure to afford the title compound, which was used in next step without further purification. LCMS: 357.9 [M+H]⁺.

### Intermediate-49: 10-(1-(benzyloxy)ethyl)-6,7-dihydro-5H-isoxazolo[4′,5′:4,5]oxocino[2,3-c]pyrazolo[1,5-a]pyridin-2-amine

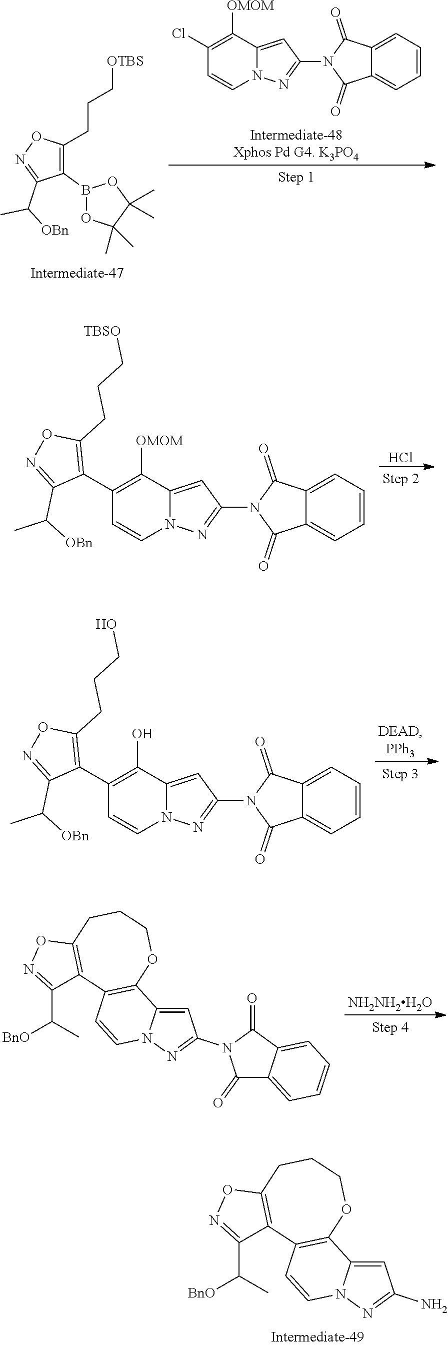

Step 1. Synthesis of 2-(5-(3-(1-(benzyloxy)ethyl)-5-(3-((tert-butyldimethylsilyl)oxy)propyl)isoxazol-4-yl)-4-(methoxymethoxy)pyrazolo[1,5-a]pyridin-2-yl)isoindoline-1,3-dione. To a solution of 2-(5-chloro-4-(methoxymethoxy)pyrazolo[1,5-a]pyridin-2-yl)isoindoline-1,3-dione (Intermediate 48) (1.10 g, 3.07 mmol, 1.0 eq) in 1,4-dioxane (20 mL) and H₂O (2 mL) was added 3-(1-(benzyloxy)ethyl)-5-(3-((tert-butyldimethylsilyl)oxy)propyl)-4-(4,4,5,5-tetramethyl-1,3,2-dioxaborolan-2-yl)isoxazole (Intermediate-47) (1.54 g, 3.07 mmol, 1.0 eq), K₃PO₄ (1.31 g, 6.15 mmol, 2.0 eq) and Xphos Pd G4 (265 mg, 307 μmol, 0.1 eq), and the resulting mixture was stirred at 40° C. for 3 h under N₂ atmosphere. The reaction mixture was quenched with water (30 mL) and extracted with EtOAc (30 mL×3). The combined organic layers were washed with brine (20 mL), dried over anhydrous Na₂SO₄, filtered and the filtrate was concentrated under reduced pressure. The residue was purified by flash silica gel chromatography (eluent of EtOAc/PE) to afford the title compound. LCMS: 697.3 [M+H]⁺.

Step 2. Synthesis of 2-(5-(3-(1-(benzyloxy)ethyl)-5-(3-hydroxypropyl)isoxazol-4-yl)-4-hydroxypyrazolo[1,5-a]pyridin-2-yl)isoindoline-1,3-dione. To 2-(5-(3-(1-(benzyloxy)ethyl)-5-(3-((tert-butyldimethylsilyl)oxy)propyl)isoxazol-4-yl)-4-(methoxymethoxy)pyrazolo[1,5-a]pyridin-2-yl)isoindoline-1,3-dione (850 mg, 1.22 mmol, 1.0 eq) was added a solution of HCl in 1,4-dioxane (2 M, 4.89 mL), and the resulting mixture was stirred at room temperature for 0.5 h. The reaction mixture was concentrated under reduced pressure, the residue was diluted with H₂O (10 mL), and then extracted with DCM (10 mL×2). The combined organic layers were washed with saturated aqueous NaHCO₃ (10 mL) and brine (10 mL), dried over anhydrous Na₂SO₄, filtered and the filtrate was concentrated under reduced pressure. The residue was purified by flash silica gel chromatography (eluent of EtOAc/PE) to afford the title compound. LCMS: 539.2 [M+H]⁺.

Step 3. Synthesis of 2-(10-(1-(benzyloxy)ethyl)-6,7-dihydro-5H-isoxazolo[4′,5′:4,5]oxocino[2,3-c]pyrazolo[1,5-a]pyridin-2-yl)isoindoline-1,3-dione. To a solution of PPh₃ (1.07 g, 4.09 mmol, 4.0 eq) in THF (10 mL) was added DEAD (658 mg, 3.78 mmol, 3.7 eq) at 0° C., and it was stirred at room temperature for 0.5 h. 2-(5-(3-(1-(benzyloxy)ethyl)-5-(3-hydroxypropyl)isoxazol-4-yl)-4-hydroxypyrazolo[1,5-a]pyridin-2-yl)isoindoline-1,3-dione (550 mg, 1.02 mmol, 1.0 eq) was added into the reaction at 5° C., and the reaction was stirred at room temperature for 0.5 h under N₂ atmosphere after addition. The reaction mixture was diluted with H₂O (30 mL), and then extracted with EtOAc (30 mL×2), the combined organic layers were washed brine (20 mL), dried over anhydrous Na₂SO₄, filtered and the filtrate was concentrated under reduced pressure. The residue was purified by flash silica gel chromatography (eluent of EtOAc/PE) to afford the title compound. LCMS: 521.1 [M+H]⁺.

Step 4. Synthesis of 10-(1-(benzyloxy)ethyl)-6,7-dihydro-5H-isoxazolo[4′,5′:4,5]oxocino[2,3-c]pyrazolo[1,5-a]pyridin-2-amine. To a solution of 2-(10-(1-(benzyloxy)ethyl)-6,7-dihydro-5H-isoxazolo[4′,5′:4,5]oxocino[2,3-c]pyrazolo[1,5-a]pyridin-2-yl)isoindoline-1,3-dione (300 mg, 576 μmol, 1.0 eq) in EtOH (2 mL) was added NH₂NH₂*H₂O (610 mg, 11.94 mmol, 20.72 eq), and the resulting mixture was stirred at room temperature for 0.5 h. The reaction mixture was diluted with H₂O (20 mL), and then extracted with EtOAc (20 mL×3), the combined organic layers were washed brine (20 mL), dried over anhydrous Na₂SO₄, filtered and the filtrate was concentrated under reduced pressure. The residue was purified by flash silica gel chromatography (eluent of EtOAc/PE) to afford the title compound. LCMS: 391.0 [M+H]⁺.

### Intermediate-51: rac-(2aR,13aS)-6-methyl-1,2,2a,13a-tetrahydro-6H-cyclobuta[2,3]pyrazolo[3′,4′:7,8][1,4]dioxocino[5,6-c]pyrazolo[1,5-a]pyridin-11-amine

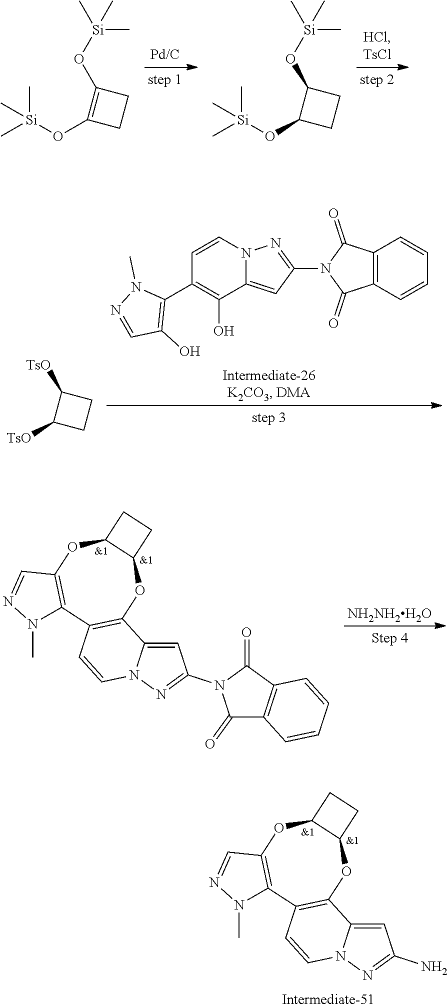

Step 1. Synthesis of (1R,2S)-1,2-bis((trimethylsilyl)oxy)cyclobutene. To a solution of 1,2-bis((trimethylsilyl)oxy)cyclobut-1-ene (20.00 g, 86.79 mmol, 1.0 eq) in THF (600 mL) was added Pd/Al₂O₃ (5% wt, 5 g), the reaction vessel was purged with H₂ gas (1.5 Mpa) and the reaction mixture was stirred under H₂ atmosphere at 80.0° C. overnight. The reaction mixture was filtered and the filtrate was concentrated under reduced pressure to afford the title compound, which was used in the next step without further purification. ¹H NMR (400 MHz, CDCl₃) δ 4.25 (br d, 2H), 1.98 (br d, 4H), 0.14 (d, 18H).

Step 2. Synthesis of (1R,2S)-cyclobutane-1,2-diyl bis(4-methylbenzenesulfonate). To a solution of HCl in 1,4-dioxane (2 M, 150 mL) was added (1R,2S)-1,2-bis((trimethylsilyl)oxy)cyclobutane (19.00 g, 81.73 mmol, 1.0 eq), and the resulting mixture was stirred at room temperature under N₂ for 1 h. The reaction mixture was concentrated under reduced pressure to give a residue. The residue was dissolved in DCM (70 mL), TEA (28.48 g, 281.48 mmol, 4.0 eq), TsCl (33.54 g, 175.93 mmol, 2.5 eq) and DMAP (860 mg, 7.04 mmol, 0.1 eq) was added, and the resulting mixture was stirred at room temperature under N₂ for 1 h. The reaction mixture was diluted with H₂O (100 mL) and extracted with DCM (200 mL×2). The combined organic layers were dried over anhydrous Na₂SO₄, filtered and the filtrate was concentrated under reduced pressure. The residue was purified by flash silica gel chromatography (eluent of EtOAc/PE) to afford the title compound. ¹H NMR (400 MHz, CDCl₃) δ 7.77 (d, 4H), 7.34 (d, 4H), 4.98-4.77 (m, 2H), 2.47 (s, 6H), 2.35-2.06 (m, 4H).

Step 3. Synthesis of rac-2-((2aR,13aS)-6-methyl-1,2,2a,13a-tetrahydro-6H-cyclobuta[2,3]pyrazolo[3′,4′:7,8][1,4]dioxocino[5,6-c]pyrazolo[1,5-a]pyridin-11-yl)isoindoline-1,3-dione. To a solution of 2-(4-hydroxy-5-(4-hydroxy-1-methyl-1H-pyrazol-5-yl)pyrazolo[1,5-a]pyridin-2-yl)isoindoline-1,3-dione (Intermediate-26) (1.00 g, 2.66 mmol, 1.0 eq) and (1R,2S)-cyclobutane-1,2-diyl bis(4-methylbenzenesulfonate) (2.11 g, 5.33 mmol, 2.0 eq) in DMA (20 mL) was added K₂CO₃ (1.84 g, 13.32 mmol, 5.0 eq), and the resulting mixture was stirred at 140° C. overnight. After cooling down to room temperature, HATU (1.71 g, 4.49 mmol, 1.0 eq) was added, and the resulting mixture was stirred at room temperature under N₂ for 1 h. The reaction mixture was diluted with saturated aqueous citric acid (200 mL) and extracted with EtOAc (100 mL×3). The combined organic layers were washed with brine (100 mL×2), dried over anhydrous Na₂SO₄, filtered and the filtrate was concentrated under reduced pressure to afford the title compound, which was used in the next step without further purification. LCMS: 428.2 [M+H]⁺.

Step 4. Synthesis of rac-(2aR,13aS)-6-methyl-1,2,2a,13a-tetrahydro-6H-cyclobuta[2,3]pyrazolo[3′,4′:7,8][1,4]dioxocino[5,6-c]pyrazolo[1,5-a]pyridin-11-amine. To a solution of rac-2-((2aR,13aS)-6-methyl-1,2,2a,13a-tetrahydro-6H-cyclobuta[2,3]pyrazolo[3′,4′:7,8][1,4]dioxocino[5,6-c]pyrazolo[1,5-a]pyridin-11-yl)isoindoline-1,3-dione (1.8 g, 4.21 mmol, 1.0 eq) in EtOH (18 mL) was added hydrazine hydrate (3.09 g, 61.73 mmol, 14.66 eq), and the resulting mixture was stirred at room temperature for 1 h. The reaction mixture was poured into water (30 mL) and extracted with EtOAc (30 mL×3). The combined organic layers were washed with brine (30 mL), dried over anhydrous Na₂SO₄, filtered and the filtrate was concentrated under reduced pressure. The residue was purified by reverse phase HPLC (C18 column, water (0.1% FA)-ACN) to afford the title compound. LCMS: 298.2 [M+H]⁺. Intermediate 52: 7-methyl-3-((methylthio)methyl)-2,3-dihydro-7H-pyrazolo[1,5-a]pyrazolo[3′,4′:7,8][1,4]dioxocino[5,6-c]pyridin-12-amine

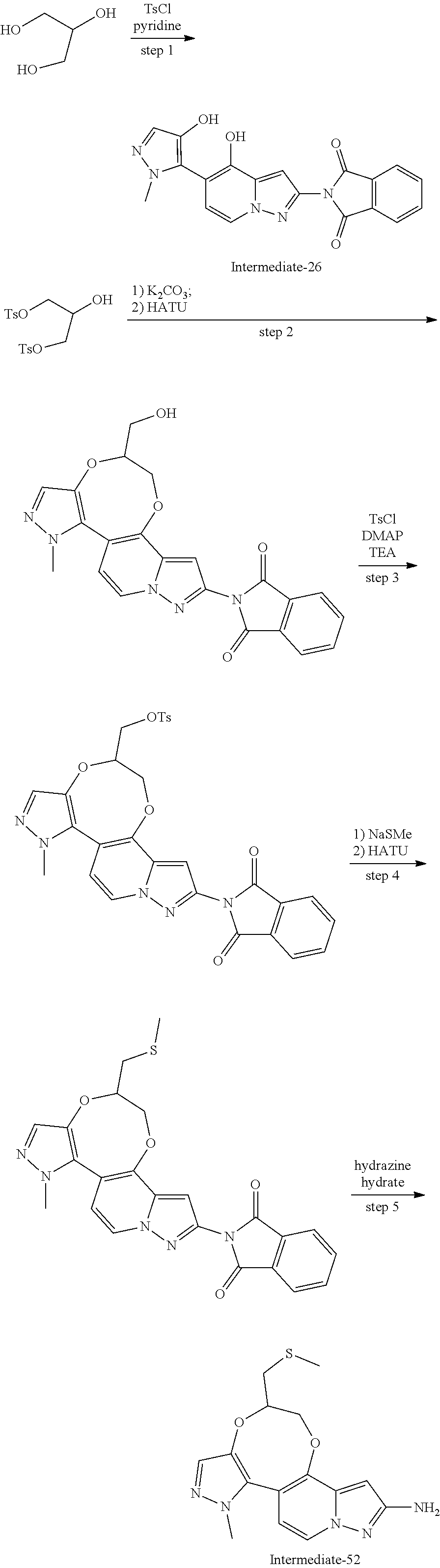

Step 1. Synthesis of 2-hydroxypropane-1,3-diyl bis(4-methylbenzenesulfonate). To a solution of propane-1,2,3-triol (10.00 g, 108.59 mmol, 8.12 mL, 1.0 eq) in pyridine (80 mL) was added TsCl (51.75 g, 271.46 mmol, 2.5 eq) at 0° C., and the reaction mixture was stirred at room temperature overnight. The reaction mixture was poured into aqueous HCl (6 N, 300 mL) and extracted with DCM (300 mL×3). The combined organic layers were washed with brine (100 mL), dried over anhydrous Na₂SO₄, filtered and the filtrate was concentrated under reduced pressure. The residue was purified by flash silica gel chromatography (Eluent of EtOAc/DCM), followed by reverse phase HPLC (C18 column, water (0.1% FA)-ACN) to afford the title compound. LCMS: 401.1 [M+H]⁺.

Step 2. Synthesis of 2-(3-(hydroxymethyl)-7-methyl-2, 3-dihydro-7H-pyrazolo[1, 5-a]pyrazolo[3′, 4′:7, 8][1, 4]dioxocino[5, 6-c]pyridin-12-yl) isoindoline-1, 3-dione. To a solution of 2-(4-hydroxy-5-(4-hydroxy-1-methyl-1H-pyrazol-5-yl) pyrazolo[1,5-a]pyridin-2-yl) isoindoline-1,3-dione (Intermediate-26) (4.00 g, 10.66 mmol, 1.0 eq) and 2-hydroxypropane-1,3-diyl bis (4-methylbenzenesulfonate) (8.54 g, 21.31 mmol, 2.0 eq) in DMA (60 mL) was added K₂CO₃ (8.84 g, 63.94 mmol, 6.0 eq), and the reaction mixture was stirred at 140° C. for 6 h. Another batch of 2-hydroxypropane-1, 3-diyl bis (4-methylbenzenesulfonate) (2.13 g, 5.33 mmol, 0.5 eq) was added and the resulting mixture was stirred at 140° C. overnight. After cooling to room temperature, HATU (4.06 g, 10.68 mmol, 1.2 eq) was added, and the reaction mixture was stirred at room temperature for 1 h. The reaction mixture was poured to saturated citric acid (1000 mL) and extracted with EtOAc (600 mL×3). The combined organic layers were washed with brine (1000 mL), dried over anhydrous Na₂SO₄, filtered and the filtrate was concentrated under reduced pressure. The residue was purified by flash silica gel chromatography (Eluent of EtOAc/PE) to afford the title compound. LCMS: 432.1 [M+H]⁺.

Step 3. Synthesis of (12-(1, 3-dioxoisoindolin-2-yl)-7-methyl-2, 3-dihydro-7H-pyrazolo[1, 5-a]pyrazolo[3′, 4′:7, 8][1, 4]dioxocino[5, 6-c]pyridin-3-yl) methyl 4-methylbenzenesulfonate. To a solution of 2-(3-(hydroxymethyl)-7-methyl-2, 3-dihydro-7H-pyrazolo[1, 5-a]pyrazolo[3′, 4′:7, 8][1, 4]dioxocino[5, 6-c]pyridin-12-yl) isoindoline-1, 3-dione (300 mg, 695 μmol, 1.0 eq) and TEA (281 mg, 2.78 mmol, 4.0 eq) in DCM (6 mL) was added TsCl (265 mg, 1.39 mmol, 2.0 eq) and DMAP (8 mg, 69 μmol, 0.1 eq) at 0° C., and the resulting mixture was stirred at room temperature for 2 h. The reaction mixture was poured into H₂O (20 mL) and extracted with DCM (20 mL×3). The combined organic layers were washed with brine (20 mL), dried over anhydrous Na₂SO₄, filtered and the filtrate was concentrated under reduced pressure. The residue was purified by flash silica gel chromatography (Eluent of EtOAc/PE) to afford the title compound. LCMS: 586.3 [M+H]⁺.

Step 4. Synthesis of 2-(7-methyl-3-((methylthio) methyl)-2, 3-dihydro-7H-pyrazolo[1, 5-a]pyrazolo[3′, 4′:7, 8][1, 4]dioxocino[5, 6-c]pyridin-12-yl) isoindoline-1, 3-dione. To a solution of (12-(1, 3-dioxoisoindolin-2-yl)-7-methyl-2, 3-dihydro-7H-pyrazolo[1,5-a]pyrazolo[3′, 4′:7, 8][1, 4]dioxocino[5, 6-c]pyridin-3-yl) methyl 4-methylbenzenesulfonate (220 mg, 376 μmol, 1.0 eq) in THF (3 mL) was added NaSMe (380 mg, 5.42 mmol, 14.4 eq) at 0° C., and the reaction mixture was stirred at 45° C. overnight. The reaction mixture was poured into saturated citric acid (20 mL) and extracted with EtOAc (15 mL×3). The combined organic layers were washed with brine (20 mL), dried over anhydrous Na₂SO₄, filtered and the filtrate was concentrated under reduced pressure to afford the residue. To the residue was added DMF (2 mL) followed by HATU (162 mg, 425 μmol, 1.2 eq) and DIEA (137 mg, 1.06 mmol, 3.0 eq), and the resulting mixture was stirred at room temperature for 2 h. The reaction mixture was poured into saturated citric acid (40 mL) and extracted with EtOAc (30 mL×3). The combined organic layers were washed with brine (60 mL), dried over anhydrous Na₂SO₄, filtered and the filtrate was concentrated under reduced pressure to afford the title compound, which was used into the next step without further purification. LCMS: 480.3 [M+H]⁺.

Step 5. Synthesis of 7-methyl-3-((methylthio) methyl)-2, 3-dihydro-7H-pyrazolo[1, 5-a]pyrazolo[3′, 4′:7, 8][1, 4]dioxocino[5, 6-c]pyridin-12-amine. To a solution of 2-(7-methyl-3-((methylthio) methyl)-2, 3-dihydro-7H-pyrazolo[1, 5-a]pyrazolo[3′, 4′:7, 8][1, 4]dioxocino[5, 6-c]pyridin-12-yl) isoindoline-1, 3-dione (200 mg, 433 μmol, 1.0 eq) in EtOH (2 mL) was added hydrazine hydrate (770 mg, 15.07 mmol, 98 wt %, 34.8 eq), and the reaction mixture was stirred at room temperature for 0.5 h. The reaction mixture was quenched with H₂O (20 mL) and extracted with EtOAc (20 mL×4). The combined organic layers were washed with brine (100 mL), dried over anhydrous Na₂SO₄, filtered and the filtrate was concentrated under reduced pressure. The residue was purified by flash silica gel chromatography (Eluent of EtOAc/PE) to afford the title compound. ¹H NMR (400 MHz, CDCl₃) δ 8.08 (d, 1H), 7.42 (s, 1H), 6.48 (d, 1H), 5.93 (s, 1H), 4.65-3.96 (m, 3H), 3.89 (s, 3H), 2.81-2.71 (m, 1H), 2.69-2.58 (m, 1H), 2.22 (s, 3H).

### Intermediate-53: 11-methyl-6-(methylthio)-6,7-dihydro-5H,11H-pyrazolo[1,5-a]pyrazolo[3′,4′:8,9][1,5]dioxonino[6,7-c]pyridin-2-amine

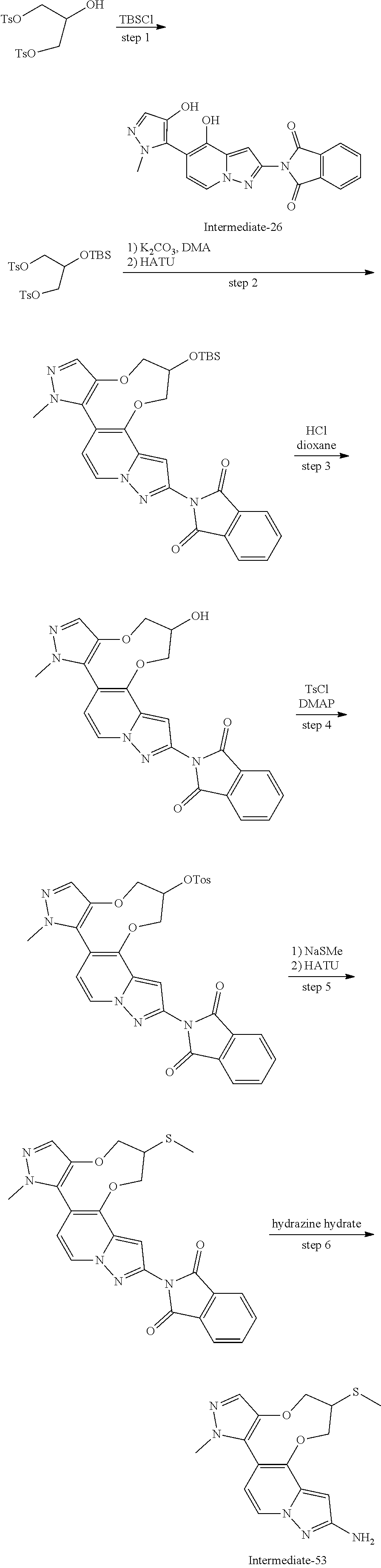

Step 1. Synthesis of 2-((tert-butyldimethylsilyl)oxy)propane-1,3-diyl bis(4-methylbenzenesulfonate). To a solution of 2-hydroxypropane-1,3-diyl bis(4-methylbenzenesulfonate) (prepared as described in step 1 of intermediate-52) (8 g, 19.98 mmol, 1.0 eq) and imidazole (2.72 g, 39.95 mmol, 2.0 eq) in DCM (60 mL) was added TBSCl (3.61 g, 23.97 mmol, 1.2 eq) and DMAP (244 mg, 2.00 mmol, 0.1 eq) at 0° C. and the resulting mixture was stirred at room temperature overnight. The reaction mixture was poured into H₂O (200 mL) and extracted with DCM (200 mL×3). The combined organic layers were washed with brine (500 mL), dried over anhydrous Na₂SO₄, filtered and the filtrate was concentrated under reduced pressure. The residue was purified by flash silica gel chromatography (Eluent of EtOAc/PE) to afford the title compound. LCMS: 515.2 [M+H]⁺.

Step 2. Synthesis of 2-(6-((tert-butyldimethylsilyl)oxy)-11-methyl-6,7-dihydro-5H,11H-pyrazolo[1,5-a]pyrazolo[3′,4′:8,9][1,5]dioxonino[6,7-c]pyridin-2-yl)isoindoline-1,3-dione. To a solution of 2-(4-hydroxy-5-(4-hydroxy-1-methyl-1H-pyrazol-5-yl)pyrazolo[1,5-a]pyridin-2-yl)isoindoline-1,3-dione (Intermediate-26) (2.7 g, 7.19 mmol, 1.0 eq) and 2-((tert-butyldimethylsilyl)oxy)propane-1,3-diyl bis(4-methylbenzenesulfonate) (6.29 g, 12.23 mmol, 1.7 eq) in DMA (40 mL) was added K₂CO₃ (5.97 g, 43.16 mmol, 6.0 eq), and the reaction mixture was stirred at 140° C. for 2 h. After cooling down to room temperature, HATU (3.64 g, 9.58 mmol, 1.2 eq) was added to the mixture, and it was stirred at room temperature for 1 h. The reaction mixture was poured into saturated citric acid (400 mL) and extracted with EtOAc (200 mL×3). The combined organic layers were washed with brine (400 mL), dried over anhydrous Na₂SO₄, filtered and the filtrate was concentrated under reduced pressure. The residue was purified by flash silica gel chromatography (Eluent of EtOAc/PE) to afford the title compound. LCMS: 546.2 [M+H]⁺.

Step 3. Synthesis of 2-(6-hydroxy-11-methyl-6,7-dihydro-5H,11H-pyrazolo[1,5-a]pyrazolo[3′,4′:8,9][1,5]dioxonino[6,7-c]pyridin-2-yl)isoindoline-1,3-dione. To a solution of HCl in 1,4-dioxane (2 M, 30 mL) was added 2-(6-((tert-butyldimethylsilyl)oxy)-11-methyl-6,7-dihydro-5H,11H-pyrazolo[1,5-a]pyrazolo[3′,4′:8,9][1,5]dioxonino[6,7-c]pyridin-2-yl)isoindoline-1,3-dione (2.4 g, 4.40 mmol, 1.0 eq) and the reaction mixture was stirred at room temperature for 1 h. The mixture was concentrated under reduced pressure to afford the title compound, which was used into the next step without further purification. LCMS: 432.2 [M+H]⁺.

Step 4. Synthesis of 2-(1,3-dioxoisoindolin-2-yl)-11-methyl-6,7-dihydro-5H,11H-pyrazolo[1,5-a]pyrazolo[3′,4′:8,9][1,5]dioxonino[6,7-c]pyridin-6-yl 4-methylbenzenesulfonate. To a solution of 2-(6-hydroxy-11-methyl-6,7-dihydro-5H,11H-pyrazolo[1,5-a]pyrazolo[3′,4′:8,9][1,5]dioxonino[6,7-c]pyridin-2-yl)isoindoline-1,3-dione (1.9 g, 4.40 mmol, 1.0 eq) and TEA (2.23 g, 22.02 mmol, 5.0 eq) in DCM (20 mL) was added TsCl (1.68 g, 8.81 mmol, 2.0 eq) and DMAP (54 mg, 440 μmol, 0.1 eq) at 0° C. and the reaction mixture was stirred at 40° C. overnight. The reaction mixture was poured into H₂O (20 mL) and extracted with DCM (40 mL×3). The combined organic layers were washed with brine (100 mL), dried over anhydrous Na₂SO₄, filtered and the filtrate was concentrated under reduced pressure. The residue was purified by flash silica gel chromatography (Eluent of EtOAc/PE) to afford the title compound. LCMS: 586.2 [M+H]⁺.

Step 5. Synthesis of 2-(11-methyl-6-(methylthio)-6,7-dihydro-5H, 11H-pyrazolo[1, 5-a]pyrazolo[3′, 4′:8, 9][1, 5]dioxonino[6, 7-c]pyridin-2-yl) isoindoline-1, 3-dione. To a solution of 2-(1,3-dioxoisoindolin-2-yl)-11-methyl-6,7-dihydro-5H,11H-pyrazolo[1,5-a]pyrazolo[3′,4′:8,9][1,5]dioxonino[6,7-c]pyridin-6-yl 4-methylbenzenesulfonate (1.00 g, 1.71 mmol, 1.0 eq) in THF (10 mL) was added NaSMe (2.02 g, 28.82 mmol, 16.9 eq) at 0° C., and the resulting mixture was stirred at 60° C. overnight. The reaction mixture was poured into saturated citric acid (100 mL) and extracted with EtOAc (80 mL×3). The combined organic layers were washed with brine (80 mL), dried over anhydrous Na₂SO₄, filtered and the filtrate was concentrated under reduced pressure to afford the residual. To residual was dissolved into DMF (8 mL), HATU (634 mg, 1.67 mmol, 1.0 eq) and DIEA (647 mg, 5.01 mmol, 3 eq) were added, and the resulting mixture was stirred at room temperature for 1 h. The reaction mixture was poured into saturated citric acid (100 mL) and extracted with EtOAc (100 mL×3). The combined organic layers were washed with brine (200 mL), dried over anhydrous Na₂SO₄, filtered and the filtrate was concentrated under reduced pressure. The residue was purified by flash silica gel chromatography (Eluent of EtOAc/PE) to afford the title compound. LCMS: 462.1 [M+H]⁺.

Step 6. Synthesis of 11-methyl-6-(methylthio)-6,7-dihydro-5H,11H-pyrazolo[1,5-a]pyrazolo[3′,4′:8,9][1,5]dioxonino[6,7-c]pyridin-2-amine. To a solution of 2-(11-methyl-6-(methylthio)-6,7-dihydro-5H,11H-pyrazolo[1,5-a]pyrazolo[3′,4′:8,9][1,5]dioxonino[6,7-c]pyridin-2-yl)isoindoline-1,3-dione (800 mg, 1.73 mmol, 1.0 eq) in EtOH (7 mL) was added hydrazine hydrate (2.04 g, 39.94 mmol, 98% purity, 23.0 eq), and the resulting mixture was stirred at room temperature for 0.5 h. The reaction mixture was quenched with H₂O (50 mL) and extracted with EtOAc (30 mL×4). The combined organic layers were washed with brine (100 mL), dried over Na₂SO₄, filtered and concentrated under reduced pressure to give a residue. The residue was purified by flash silica gel chromatography (Eluent of EtOAc/PE) to afford the title compound. LCMS: 332.1 [M+H]⁺

### EXAMPLES OF THE DISCLOSURE

### Example 1. Synthesis of (1S,2R)—N-{3-methyl-7,10-dioxa-3,4,15,16-tetraazatetracyclo[9.7.0.0\hat{ }{2,6}.0\hat{ }{12,16}]octadeca-1(11),2(6),4,12,14,17-hexaen-14-yl}-2-(5-methylpyridin-2-yl)cyclopropane-1-carboxamide (I-1)

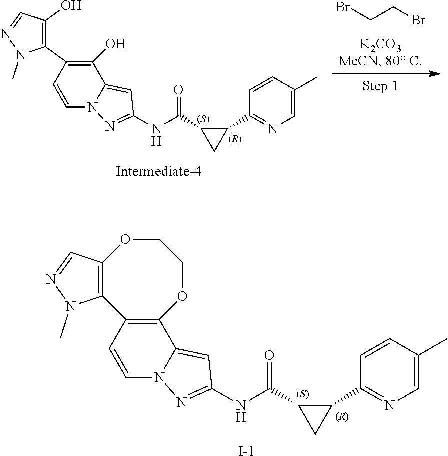

Step 1. To a solution of (1S,2R)—N-(4-hydroxy-5-(4-hydroxy-1-methyl-1H-pyrazol-5-yl)pyrazolo[1,5-a]pyridin-2-yl)-2-(5-methylpyridin-2-yl)cyclopropane-1-carboxamide (Intermediate-4) (35 mg, 86 μmol, 1 eq) in ACN (1 mL) was added K₂CO₃ (48 mg, 346 μmol, 4 eq) and 1,2-dibromoethane (16 mg, 86 μmol, 1 eq). The resulting mixture was stirred at 80° C. for 15 h. The reaction mixture was filtered and the filtrate was concentrated under reduced pressure to give a residue. The residue was purified by flash silica gel chromatography (Eluent of DCM/MeOH) and reverse phase preparative HPLC (MeCN/H₂O, 0.1% ammonia) to afford the title compound. ¹H NMR (400 MHz, CD₃OD) δ=8.28 (d, 1H), 8.23 (d, 1H), 7.58-7.50 (m, 1H), 7.37-7.24 (m, 2H), 6.83 (d, 1H), 6.77 (s, 1H), 4.52-3.90 (m, 4H), 3.84 (s, 3H), 2.77-2.66 (m, 1H), 2.47-2.35 (m, 1H), 2.28 (s, 3H), 1.92-1.84 (m, 1H), 1.54-1.44 (m, 1H). LCMS: 431.2 [M+H]⁺.

### Example 2. Synthesis of (1S,2R)—N-[(9R)-3,9-dimethyl-7,10-dioxa-3,4,15,16-tetraazatetracyclo[9.7.0.0\hat{ }{2,6}.0\hat{ }{12,16}]octadeca-1(11),2(6),4,12,14,17-hexaen-14-yl]-2-(5-methylpyridin-2-yl)cyclopropane-1-carboxamide (I-5) and (1S,2R)—N-[(8R)-3,8-dimethyl-7,10-dioxa-3,4,15,16-tetraazatetracyclo[9.7.0.0\hat{ }{2,6}0.0\hat{ }{12,16}]octadeca-1(11),2(6),4,12,14,17-hexaen-14-yl]-2-(5-methylpyridin-2-yl)cyclopropane-1-carboxamide (I-6)

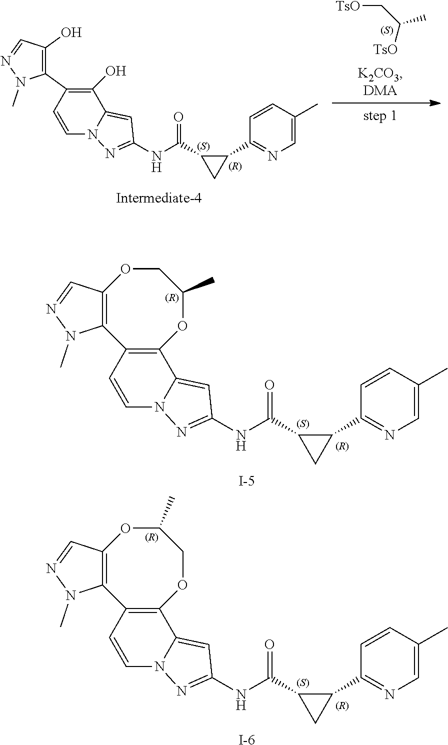

Step 1: To a solution of (1S,2R)—N-(4-hydroxy-5-(4-hydroxy-1-methyl-1H-pyrazol-5-yl)pyrazolo[1,5-a]pyridin-2-yl)-2-(5-methylpyridin-2-yl)cyclopropane-1-carboxamide (Intermediate-4) (500 mg, 1.24 mmol, 1.0 eq) and (S)-propane-1,2-diyl bis(4-methylbenzenesulfonate) (713 mg, 1.85 mmol, 1.5 eq) in DMA (6 mL) was added K₂CO₃ (683 mg, 4.95 mmol, 4.0 eq), and the resulting mixture was stirred at 120° C. for 0.5 h. The reaction mixture was filtered and the filtrate was concentrated under reduced pressure. The residue was purified by silica gel chromatography (EtOAc/PE) followed by normal phase HPLC (XB-CN column, Hexane/EtOH) to afford a mixture of (1S,2R)—N—((R)-2,7-dimethyl-2,3-dihydro-7H-pyrazolo[1,5-a]pyrazolo[3′,4′:7,8][1,4]dioxocino[5,6-c]pyridin-12-yl)-2-(5-methylpyridin-2-yl)cyclopropane-1-carboxamide and (1S,2R)—N—((R)-3,7-dimethyl-2,3-dihydro-7H-pyrazolo[1,5-a]pyrazolo[3′,4′:7,8][1,4]dioxocino[5,6-c]pyridin-12-yl)-2-(5-methylpyridin-2-yl)cyclopropane-1-carboxamide. The resulting mixture was separated by chiral SFC (instrument: Waters 80Q Preparative SFC system; column: DAICEL CHIRALPAK IK, 250×25 mm I.D., 10 μm particle size; mobile phase A: CO₂, mobile phase B: EtOH:ACN=4:1 (0.1% NH₄OH); isocratic elution: 50% phase B in supercritical CO₂; flow rate: 80 g/min; retention time: peak1 (I-5)—4.50 min, peak2 (I-6)—5.35 min; back pressure: 100 bar to keep the CO₂ in supercritical flow; wave length: 220 nm).

Peak1 was further purified by reverse phase HPLC (C18 column, water (0.1% FA)-ACN to afford (1S,2R)—N—((R)-2,7-dimethyl-2,3-dihydro-7H-pyrazolo[1,5-a]pyrazolo[3′,4′:7,8][1,4]dioxocino[5,6-c]pyridin-12-yl)-2-(5-methylpyridin-2-yl)cyclopropane-1-carboxamide (example I-5). LCMS: 445.2 [M+H]⁺. 1H NMR (400 MHz, Methanol-d4) δ 8.32-8.11 (m, 2H), 7.66-7.46 (m, 1H), 7.39-7.21 (m, 2H), 6.82 (d, 1H), 6.74 (s, 1H), 4.20 (br d, 2H), 3.83 (s, 3H), 3.66-3.44 (m, 1H), 2.79-2.63 (m, 1H), 2.48-2.38 (m, 1H), 2.28 (s, 3H), 1.92-1.80 (m, 1H), 1.57-1.48 (m, 1H), 1.41 (d, 3H).

Peak2 was further purified by reverse phase HPLC (C18 column, water (0.1% FA)-ACN to afford (1S,2R)—N—((R)-3,7-dimethyl-2,3-dihydro-7H-pyrazolo[1,5-a]pyrazolo[3′,4′:7,8][1,4]dioxocino[5,6-c]pyridin-12-yl)-2-(5-methylpyridin-2-yl)cyclopropane-1-carboxamide (example I-6). LCMS: 445.2 [M+H]⁺. 1H NMR (400 MHz, Methanol-d4) δ 8.34-8.17 (m, 2H), 7.65-7.47 (m, 1H), 7.40-7.21 (m, 2H), 6.81 (d, 1H), 6.74 (s, 1H), 4.74-3.85 (m, 3H), 3.84 (s, 3H), 2.80-2.65 (m, 1H), 2.49-2.32 (m, 1H), 2.28 (s, 3H), 1.94-1.81 (m, 1H), 1.58-1.46 (m, 1H), 1.21 (br d, 3H).

### Example 3. Synthesis of 3-methyl-N-(6-methylpyridazin-3-yl)-7,10-dioxa-3,4,15,16-tetraazatetracyclo[9.7.0.0\hat{ }{2,6}0.0\hat{ }{12,16}]octadeca-1(11),2(6),4,12,14,17-hexaen-14-amine (I-11)

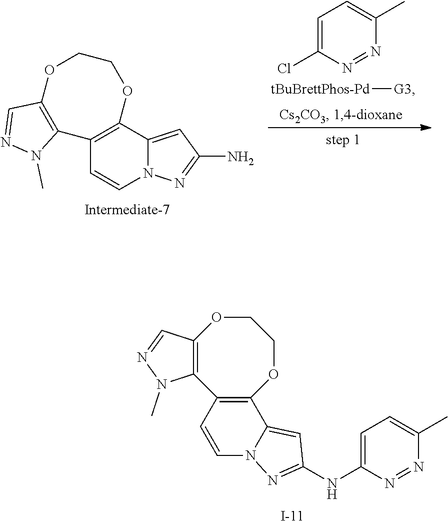

Step 1. To a solution of 7-methyl-2,3-dihydro-7H-pyrazolo[1,5-a]pyrazolo[3′,4′:7,8][1,4]-dioxocino[5,6-c]pyridin-12-amine (Intermediate-7) (30 mg, 111 μmol, 1.0 eq) and 3-chloro-6-methylpyridazine (21 mg, 166 μmol, 1.5 eq) in 1,4-dioxane (1 mL) was added t-Bu-BrettPhos-Pd-G3 (9 mg, 11 μmol, 0.1 eq) and Cs₂CO₃ (108 mg, 332 μmol, 3.0 eq), and the resulting mixture was stirred at 100° C. for 1 h. The reaction mixture was cooled to room temperature, diluted with H₂O (20 mL) and extracted with EtOAc (25 mL×4). The combined organic layers were washed with brine (20 mL), dried over anhydrous Na₂SO₄, filtered and the filtrate was concentrated under reduced pressure. The residue was purified by silica gel chromatography (EtOAc/PE), followed by reverse phase HPLC (C18 column, water (0.1% FA)-ACN to afford the title compound. LCMS: 364.1 [M+H]⁺. ¹H NMR (400 MHz, Methanol-d4) δ 8.34 (d, 1H), 7.67 (d, 1H), 7.45 (d, 1H), 7.36 (s, 1H), 7.01 (s, 1H), 6.84 (d, 1H), 4.60-3.93 (m, 4H), 3.88 (s, 3H), 2.57 (s, 3H).

### Example 4. Synthesis of 3-methyl-1-{3-methyl-7,10-dioxa-3,4,15,16-tetraazatetracyclo[9.7.0.0\hat{ }{2,6}.0\hat{ }{12,16}]octadeca-1(11),2(6),4,12,14,17-hexaen-14-yl}urea (I-12)

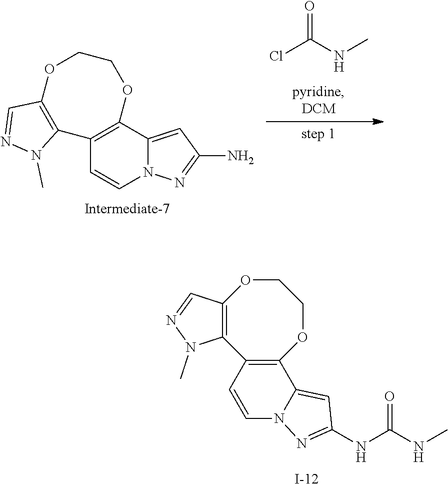

Step 1. To a solution of 7-methyl-2,3-dihydro-7H-pyrazolo[1,5-a]pyrazolo[3′,4′:7,8][1,4]-dioxocino[5,6-c]pyridin-12-amine (Intermediate-7) (30 mg, 111 μmol, 1.0 eq) in DCM (0.3 mL) and pyridine (0.3 mL) was added methylcarbamic chloride (52 mg, 553 μmol, 5.0 eq), and the resulting mixture was stirred at room temperature for 10 min. The reaction mixture was diluted with H₂O (20 mL) and extracted with EtOAc (25 mL×3). The combined organic layers were washed with brine (20 mL), dried over anhydrous Na₂SO₄, filtered and the filtrate was concentrated under reduced pressure. The residue was purified by reverse phase HPLC (C18 column, water (0.1% FA)-ACN to afford the title compound. LCMS: 329.1 [M+H]⁺. ¹H NMR (400 MHz, DMSO-d₆) δ 9.29 (s, 1H), 8.41 (d, 1H), 7.35 (s, 1H), 6.90 (d, 1H), 6.66 (s, 1H), 6.52 (br d, 1H), 4.62-3.88 (m, 4H), 3.81 (s, 3H), 2.70 (d, 3H).

### Example 5. Synthesis of (1S,2R)—N-{3-methyl-10-oxa-3,4,15,16-tetraazatetracyclo-[9.7.0.0\hat{ }{2,6}.0\hat{ }{12,16}]octadeca-1(11),2(6),4,12,14,17-hexaen-14-yl}-2-(5-methylpyridin-2-yl)cyclopropane-1-carboxamide (I-15)

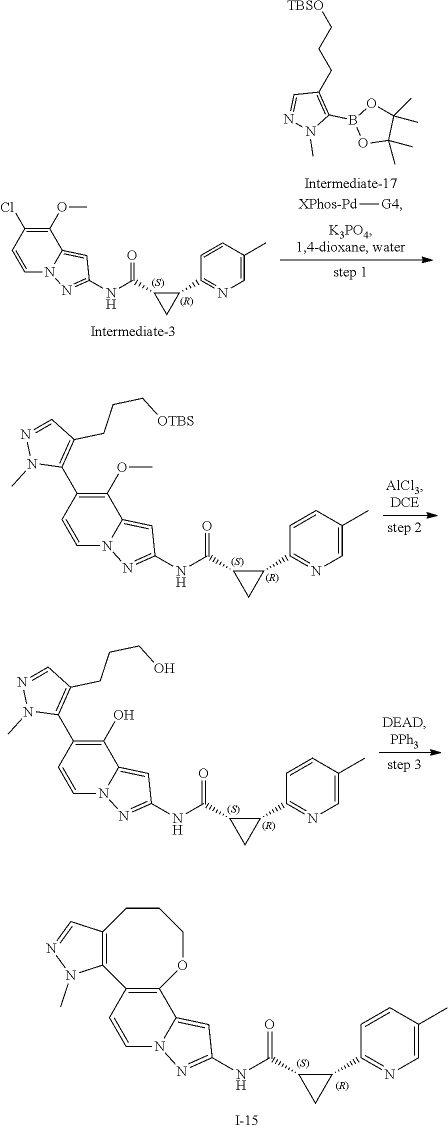

Step 1. Synthesis of (1S,2R)—N-(5-(4-(3-((tert-butyldimethylsilyl)oxy)propyl)-1-methyl-1H-pyrazol-5-yl)-4-methoxypyrazolo[1,5-a]pyridin-2-yl)-2-(5-methylpyridin-2-yl)cyclopropane-1-carboxamide. To a solution of 4-(3-((tert-butyldimethylsilyl)oxy)propyl)-1-methyl-5-(4,4,5,5-tetramethyl-1,3,2-dioxaborolan-2-yl)-1H-pyrazole (Intermediate-17) (533 mg, 1.40 mmol, 2.0 eq) and (1S,2R)—N-(5-chloro-4-methoxypyrazolo[1,5-a]pyridin-2-yl)-2-(5-methylpyridin-2-yl)cyclopropane-1-carboxamide (Intermediate-3) (250 mg, 701 μmol, 1.0 eq) in 1,4-dioxane (2 mL) and H₂O (0.4 mL) was added K₃PO₄ (446 mg, 2.10 mmol, 3.0 eq) and Xphos-Pd-G4 (60 mg, 70 μmol, 0.1 eq) and the resulting mixture was stirred at 50° C. for 1 h under N₂ atmosphere. The reaction mixture was concentrated under reduced pressure and then purified by silica gel chromatography (EtOAc/PE) to afford the title compound. LCMS: 575.2 [M+H]⁺.

Step 2. Synthesis of (1S,2R)—N-(4-hydroxy-5-(4-(3-hydroxypropyl)-1-methyl-1H-pyrazol-5-yl)pyrazolo[1,5-a]pyridin-2-yl)-2-(5-methylpyridin-2-yl)cyclopropane-1-carboxamide To a solution of (1S,2R)—N-(5-(4-(3-((tert-butyldimethylsilyl)oxy)propyl)-1-methyl-1H-pyrazol-5-yl)-4-methoxypyrazolo[1,5-a]pyridin-2-yl)-2-(5-methylpyridin-2-yl)cyclopropane-1-carboxamide (300 mg, 522 μmol, 1.0 eq) in DCE (10 mL) was added AlCl₃ (348 mg, 2.61 mmol, 5.0 eq) and the resulting mixture was stirred at 80° C. for 0.5 h. The reaction mixture was quenched with H₂O (1.5 mL) and the resulting mixture was purified directly by silica gel chromatography (EtOAc/PE), followed by reverse phase HPLC (C18 column, water (0.1% FA)-ACN to afford the title compound. LCMS: 447.1 [M+H]⁺.

Step 3. Synthesis of (1S,2R)—N-{3-methyl-10-oxa-3,4,15,16-tetraazatetracyclo[9.7.0.0\hat{ }{2,6}0.0\hat{ }{12,16}]octadeca-1(11),2(6),4,12,14,17-hexaen-14-yl}-2-(5-methylpyridin-2-yl)cyclopropane-1-carboxamide. To a solution of PPh₃ (73 mg, 280 μmol, 2.5 eq) in THF (2 mL) was added DEAD (49 mg, 280 μmol, 2.5 eq) at 0° C. and it was stirred at 0° C. for 0.25 h. (1S,2R)—N-(4-hydroxy-5-(4-(3-hydroxypropyl)-1-methyl-1H-pyrazol-5-yl)pyrazolo[1,5-a]pyridin-2-yl)-2-(5-methylpyridin-2-yl)cyclopropane-1-carboxamide (50 mg, 112 μmol, 1.0 eq) in THF (0.1 mL) was added to the reaction mixture at 0° C., the resulting mixture was stirred at 0° C. for 0.25 h. The reaction mixture was diluted with H₂O (10 mL) and extracted with EtOAc (15 mL×3). The combined organic layers were washed with brine (10 mL), dried over anhydrous Na₂SO₄, filtered, and the filtrate was concentrated under reduced pressure. The residue was purified by reverse phase HPLC (C18 column, water (0.1% FA)-ACN to afford the title compound. LCMS: 429.0 [M+H]⁺. ¹H NMR (400 MHz, Methanol-d₄) δ 8.23 (s, 1H), 8.20 (d, 1H), 7.60-7.53 (m, 1H), 7.37 (s, 1H), 7.32 (d, 1H), 6.80-6.69 (m, 2H), 4.19 (br s, 2H), 3.84 (s, 3H), 2.78-2.69 (m, 1H), 2.69-2.43 (m, 2H), 2.43-2.31 (m, 1H), 2.28 (s, 3H), 1.94-1.82 (m, 1H), 1.75 (br d, 2H), 1.56-1.45 (m, 1H).

### Example 6. Synthesis of (1S,2R)-2-{imidazo[1,2-b]pyridazin-2-yl}-N-{3-methyl-7,10-dioxa-3,4,15,16-tetraazatetracyclo[9.7.0.0\hat{ }{2,6}.0\hat{ }{12,16}]octadeca-1(11),2(6),4,12,14,17-hexaen-14-yl}cyclopropane-1-carboxamide (I-17)

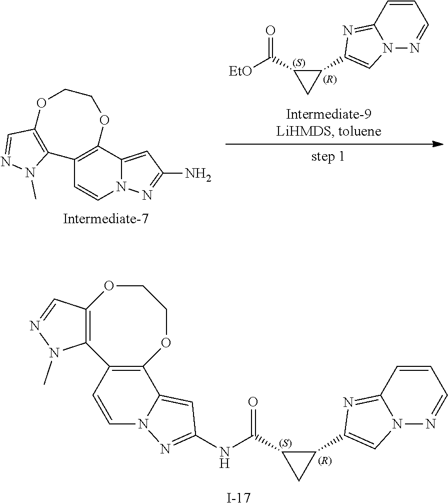

Step 1. To a solution of ethyl (1S,2R)-2-(imidazo[1,2-b]pyridazin-2-yl)cyclopropane-1-carboxylate (Intermediate-9) (31 mg, 133 μmol, 1.2 eq) and 7-methyl-2,3-dihydro-7H-pyrazolo[1,5-a]pyrazolo[3′,4′:7,8][1,4]dioxocino[5,6-c]pyridin-12-amine (Intermediate-7) (30 mg, 111 μmol, 1.0 eq) in toluene (1 mL) was added LiHMDS (1 M in THF, 221 μL, 2.0 eq), and the resulting mixture was stirred 40° C. for 1 h. The reaction mixture cooled to room temperature, quenched with saturated NH₄Cl (20 mL) and extracted with EtOAc (20 mL×3). The combined organic layers were washed with brine (20 mL), dried over anhydrous Na₂SO₄, filtered, and the filtrate was concentrated under reduced pressure. The residue was purified by reverse phase HPLC (C18 column, H₂O (10 mmol/L NH₄HCO₃)-ACN) to afford the title compound. LCMS: 457.2 [M+H]⁺. ¹H NMR (400 MHz, CDCl₃) δ 9.65 (br s, 1H), 8.30-8.25 (m, 1H), 8.12 (d, 1H), 7.98-7.90 (m, 2H), 7.37 (s, 1H), 7.06-6.96 (m, 2H), 6.58 (d, 1H), 4.53-3.91 (m, 4H), 3.83 (s, 3H), 2.82-2.72 (m, 1H), 2.37-2.28 (m, 1H), 2.05-1.97 (m, 1H), 1.66 (br s, 1H)

### Example 7. Synthesis of (1S,2R)—N-{3-methyl-7,10-dioxa-3,4,12,15,18-pentaazatetracyclo[9.7.0.0\hat{ }{2,6}0.0\hat{ }{12,16}]octadeca-1(11),2(6),4,13,15,17-hexaen-14-yl}-2-(5-methylpyridin-2-yl)cyclopropane-1-carboxamide (I-40)

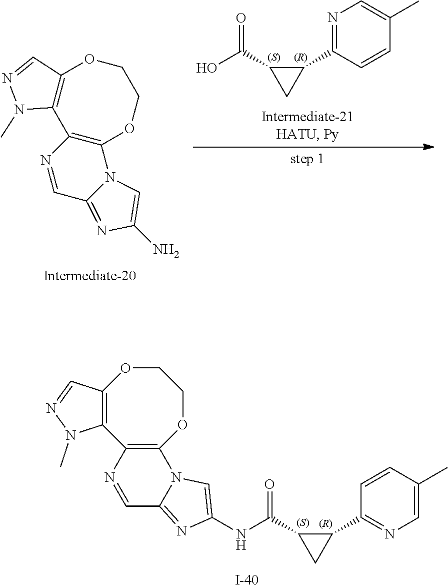

Step 1. To a solution of 11-methyl-6,7-dihydro-11H-imidazo[1,2-a]pyrazolo[3′,4′:7,8][1,4]dioxocino[6,5-e]pyrazin-2-amine (Intermediate-20) (5 mg, 18 μmol, 1.0 eq) and (1S,2R)-2-(5-methylpyridin-2-yl)cyclopropane-1-carboxylic acid (Intermediate-21) (7 mg, 37 μmol, 2.0 eq) in pyridine (0.2 mL) was added HATU (14 mg, 37 μmol, 2.0 eq), and the resulting mixture was stirred at 30° C. for 10 min. The reaction mixture was concentrated under reduced pressure and then purified by reverse phase HPLC (C18 column, water (0.1% FA)-ACN) to afford the title compound. LCMS: 432.0 [M+H]⁺. ¹H NMR (400 MHz, methanol-d4) δ 8.73 (s, 1H), 8.26 (s, 1H), 8.03 (s, 1H), 7.69-7.62 (m, 1H), 7.39 (br d, 1H), 7.34 (s, 1H), 4.69-4.45 (m, 2H), 4.21-4.06 (m, 2H), 3.97 (s, 3H), 2.83-2.72 (m, 1H), 2.51-2.45 (m, 1H), 2.32 (s, 3H), 1.95-1.87 (m, 1H), 1.60-1.51 (m, 1H).

### Example 8. Synthesis of (1S,2R)—N-{9-hydroxy-3,9-dimethyl-7,11-dioxa-3,4,16,17-tetraazatetracyclo[10.7.0.0\hat{ }{2,6}0.0\hat{ }{13,17}]nonadeca-1(12),2(6),4,13,15,18-hexaen-15-yl}-2-(5-methylpyridin-2-yl)cyclopropane-1-carboxamide (mixture of stereoisomers, I-19), (1S,2R)—N-[(9R*)-9-hydroxy-3,9-dimethyl-7,11-dioxa-3,4,16,17-tetraazatetracyclo[10.7.0.0\hat{ }{2,6}0.0\hat{ }{13,17}]nonadeca-1(12),2(6),4,13,15,18-hexaen-15-yl]-2-(5-methylpyridin-2-yl)cyclopropane-1-carboxamide (first eluting stereoisomer, I-42) and (1S,2R)—N-[(9R*)-9-hydroxy-3,9-dimethyl-7,11-dioxa-3,4,16,17-tetraazatetracyclo[10.7.0.0\hat{ }{2,6}0.0\hat{ }{13,17}]nonadeca-1(12),2(6),4,13,15,18-hexaen-15-yl]-2-(5-methylpyridin-2-yl)cyclopropane-1-carboxamide (Second Eluting Stereoisomer, I-43)

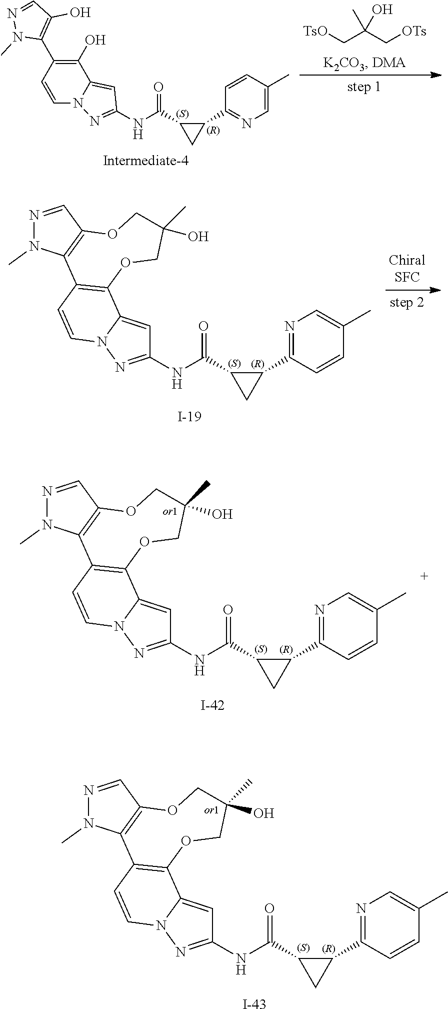

Step 1: Synthesis of (1S,2R)—N-{9-hydroxy-3,9-dimethyl-7,11-dioxa-3,4,16,17-tetraazatetracyclo[10.7.0.0\hat{ }{2,6}0.0\hat{ }{13,17}]nonadeca-1(12),2(6),4,13,15,18-hexaen-15-yl}-2-(5-methylpyridin-2-yl)cyclopropane-1-carboxamide (I-19). To a solution of (1S,2R)—N-(4-hydroxy-5-(4-hydroxy-1-methyl-1H-pyrazol-5-yl)pyrazolo[1,5-a]pyridin-2-yl)-2-(5-methylpyridin-2-yl)cyclopropane-1-carboxamide (Intermediate-4) (500 mg, 1.24 mmol, 1.0 eq) and 2-hydroxy-2-methylpropane-1,3-diyl bis(4-methylbenzenesulfonate) (1.02 g, 2.47 mmol, 2.0 eq) in DMA (10 mL) was added K₂CO₃ (684 mg, 4.95 mmol, 4.0 eq), and the resulting mixture was stirred at 120° C. for 0.5 h. After cooling down to room temperature, the reaction mixture was filtered and the filter cake was washed with DCM (30 mL). The combined filtrate was concentrated under reduced pressure and then purified by reverse phase HPLC (C18 column, H₂O (10 mmol/L NH₄HCO₃)-ACN) to afford the title compound.

Step 2. Synthesis of (1S,2R)—N-[(9R*)-9-hydroxy-3,9-dimethyl-7,11-dioxa-3,4,16,17-tetraazatetracyclo[10.7.0.0\hat{ }{2,6}0.0\hat{ }{13,17}]nonadeca-1(12),2(6),4,13,15,18-hexaen-15-yl]-2-(5-methylpyridin-2-yl)cyclopropane-1-carboxamide (I-42) and (1S,2R)—N-[(9R*)-9-hydroxy-3,9-dimethyl-7,11-dioxa-3,4,16,17-tetraazatetracyclo[10.7.0.0\hat{ }{2,6}.0\hat{ }\hat{ }{13,17}]nonadeca-1(12),2(6),4,13,15,18-hexaen-15-yl]-2-(5-methylpyridin-2-yl)cyclopropane-1-carboxamide (I-43). (1S,2R)—N-{9-hydroxy-3,9-dimethyl-7,11-dioxa-3,4,16,17-tetraazatetracyclo[10.7.0.0\hat{ }{2,6}.0\hat{ }{13,17}]nonadeca-1(12),2(6),4,13,15,18-hexaen-15-yl}-2-(5-methylpyridin-2-yl)cyclopropane-1-carboxamide (I-19) was separated by chiral SFC (instrument: Waters 80Q Preparative SFC system; column: DAICEL CHIRALPAK AD (250 mm*30 mm, 10 μm); mobile phase A: CO₂, mobile phase B: EtOH:ACN=1:1(0.1% NH₄OH); Isocratic elution: 70% phase B in supercritical CO₂; flow rate: 80 g/min; retention time: peak1 (I-42)—1.77 min, peak2 (I-43)—3.00 min; back pressure: 100 bar; wave length: 220 nm) to afford two peaks.

Peak1 was further purified by reverse phase HPLC (C18 column, water (0.1% FA)-ACN to afford (1S,2R)—N-[(9R*)-9-hydroxy-3,9-dimethyl-7,11-dioxa-3,4,16,17-tetraazatetracyclo[10.7.0.0\hat{ }{2,6}.0\hat{ }{13,17}]nonadeca-1(12),2(6),4,13,15,18-hexaen-15-yl]-2-(5-methylpyridin-2-yl)cyclopropane-1-carboxamide (first eluting stereoisomer, I-42). LCMS: 475.3 [M+H]⁺. ¹H NMR (400 MHz, Methanol-d₄) δ 8.35-8.20 (m, 2H), 7.60 (br d, 1H), 7.41 (s, 1H), 7.34 (br d, 1H), 6.81 (s, 1H), 6.76 (d, 1H), 4.33-3.98 (m, 4H), 3.81 (s, 3H), 2.73 (d, 1H), 2.51-2.37 (m, 1H), 2.30 (s, 3H), 1.94-1.85 (m, 1H), 1.52 (br d, 1H), 1.19 (s, 3H).

Peak2 was further purified by reverse phase HPLC (C18 column, water (0.1% FA)-ACN to afford (1S,2R)—N-[(9R*)-9-hydroxy-3,9-dimethyl-7,11-dioxa-3,4,16,17-tetraazatetracyclo[10.7.0.0\hat{ }{2,6}.0\hat{ }{13,17}]nonadeca-1(12),2(6),4,13,15,18-hexaen-15-yl]-2-(5-methylpyridin-2-yl)cyclopropane-1-carboxamide (second eluting stereoisomer, I-43). LCMS: 475.3 [M+H]⁺. ¹H NMR (400 MHz, Methanol-d₄) δ 8.31-8.13 (m, 2H), 7.64-7.51 (m, 1H), 7.41 (s, 1H), 7.32 (d, 1H), 6.80 (s, 1H), 6.75 (d, 1H), 4.31-3.96 (m, 4H), 3.81 (s, 3H), 2.80-2.63 (m, 1H), 2.41 (br d, 1H), 2.29 (s, 3H), 1.88 (br d, 1H), 1.59-1.46 (m, 1H), 1.20 (s, 3H).

### Example 9. Synthesis of (1S,2R)—N-{3,7-dimethyl-8-oxo-10-oxa-3,4,7,15,16-pentaazatetracyclo[9.7.0.0\hat{ }{2,6}0.0\hat{ }{12,16}]octadeca-1(11),2(6),4,12,14,17-hexaen-14-yl}-2-(5-methylpyridin-2-yl)cyclopropane-1-carboxamide (I-41)

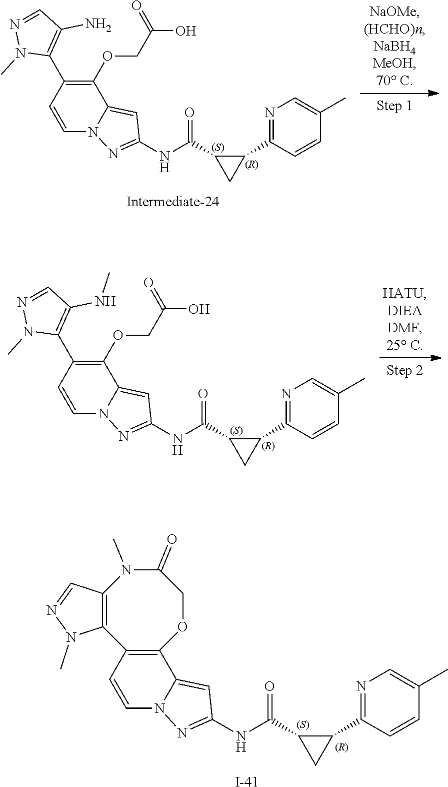

Step 1. Synthesis of 2-((5-(1-methyl-4-(methylamino)-1H-pyrazol-5-yl)-2-((1S,2R)-2-(5-methylpyridin-2-yl)cyclopropane-1-carboxamido)pyrazolo[1,5-a]pyridin-4-yl)oxy)acetic acid. To a solution of 2-((5-(4-amino-1-methyl-1H-pyrazol-5-yl)-2-((1S,2R)-2-(5-methylpyridin-2-yl)cyclopropane-1-carboxamido)pyrazolo[1,5-a]pyridin-4-yl)oxy)acetic acid hydrochloride (Intermediate-24) (88 mg, 177 μmol, 1 eq) and paraformaldehyde (32 mg, 1.06 mmol, 6 eq) in MeOH (2 mL) was added NaOMe (5.4 M, 196 μL, 6 eq) at 0° C. The resulting mixture was stirred at 70° C. for 2 h. Then the reaction mixture was cooled to 0° C. and NaBH₄ (40 mg, 1.06 mmol, 6 eq) was added under N₂ atmosphere. The resulting mixture was warmed to 70° C. and stirred for 1 h. The reaction mixture was cooled to room temperature and filtered, and the filter cake was washed with MeOH (10 mL). The combined filtrate was concentrated under reduced pressure to afford the title compound which was used in the next step without further purification. LCMS: 476.1 [M+H⁺].

Step 2. Synthesis of (1S,2R)—N-{3,7-dimethyl-8-oxo-10-oxa-3,4,7,15,16-pentaazatetracyclo[9.7.0.0\hat{ }{2,6}0.0\hat{ }{12,16}]octadeca-1(11),2(6),4,12,14,17-hexaen-14-yl}-2-(5-methylpyridin-2-yl)cyclopropane-1-carboxamide. To a solution of 2-((5-(1-methyl-4-(methylamino)-1H-pyrazol-5-yl)-2-((1S,2R)-2-(5-methylpyridin-2-yl)cyclopropane-1-carboxamido)pyrazolo[1,5-a]pyridin-4-yl)oxy)acetic acid (200 mg, 421 μmol, 1 eq) in DMF (6 mL) was added DIEA (326 mg, 2.52 mmol, 6 eq) and HATU (480 mg, 1.26 mmol, 3 eq). The mixture was stirred at 25° C. for 1 h. Then to the mixture was added H₂O (20 mL). The resulting solution was extracted with DCM (25 mL×2). The combined organic phase was washed with brine (10 mL), dried over anhydrous Na₂SO₄, filtered, and concentrated under reduced pressure. The residue was purified by reverse phase preparative HPLC (ACN/H₂O, 0.05% NH₃·H₂O) to afford the title compound. ¹H NMR (400 MHz, CDCl₃) δ=10.14-9.89 (m, 1H), 8.40 (s, 1H), 8.04 (d, J=7.2 Hz, 1H), 7.54 (s, 1H), 7.50-7.43 (m, 1H), 7.26 (s, 1H), 7.07 (s, 1H), 6.31 (d, J=7.2 Hz, 1H), 5.70-4.20 (m, 2H), 3.87 (s, 3H), 3.28 (s, 3H), 2.66 (m, 1H), 2.31 (s, 3H), 2.27 (m, 1H), 2.03 (m, 1H), 1.62 (m, 1H). LCMS: 458.3 [M+H⁺].

### Example 10. Synthesis of ((1S,2R)-2-(5-methylpyridin-2-yl)-N-[(1s,18s)-13-methyl-2,17-dioxa-7,8,13,14-tetraazapentacyclo[16.1.1.0\hat{ }{3,11}.0\hat{ }{4,8}.0\hat{ }{12,16}]icosa-3(11),4,6,9,12(16),14-hexaen-6-yl]cyclopropane-1-carboxamide (I-56)

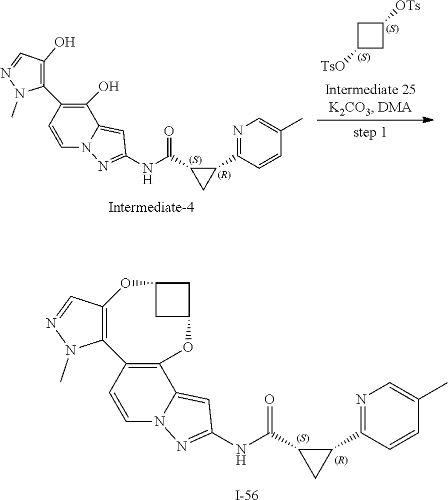

Step 1. To a solution of (1S,2R)—N-(4-hydroxy-5-(4-hydroxy-1-methyl-1H-pyrazol-5-yl)pyrazolo[1,5-a]pyridin-2-yl)-2-(5-methylpyridin-2-yl)cyclopropane-1-carboxamide (Intermediate-4) (50 mg, 124 μmol, 1.0 eq) in DMA (2 mL) was added K₂CO₃ (68 mg, 495 μmol, 4.0 eq) and (1s,3s)-cyclobutane-1,3-diyl bis(4-methylbenzenesulfonate) (Intermediate-25) (74 mg, 185.45 μmol, 1.5 eq), and the resulting mixture was stirred at 120° C. for 0.5 h. The reaction mixture was filtered and the filtrate was purified directly by reverse phase HPLC (C18 column, water (0.1% FA)-ACN to afford the title compound. LCMS: 457.4 [M+H]⁺. ¹H NMR (400 MHz, Methanol-d4) δ 8.30 (d, 1H), 8.23 (s, 1H), 7.60-7.51 (m, 1H), 7.34-7.24 (m, 2H), 6.77 (d, 1H), 6.64 (s, 1H), 5.03-4.95 (m, 1H), 4.75-4.66 (m, 1H), 3.70 (s, 3H), 2.77-2.61 (m, 3H), 2.43-2.33 (m, 1H), 2.29 (s, 3H), 2.16-1.97 (m, 2H), 1.91-1.83 (m, 1H), 1.55-1.44 (m, 1H).

### Example 11. Synthesis of (1S,2R)—N-{3-methyl-7,11-dioxa-3,4,16,17-tetraazatetracyclo-[10.7.0.0\hat{ }{2,6}.0\hat{ }{13,17}]nonadeca-1(12),2(6),4,13,15,18-hexaen-15-yl}-2-(5-methylpyrimidin-2-yl)cyclopropane-1-carboxamide (I-54)

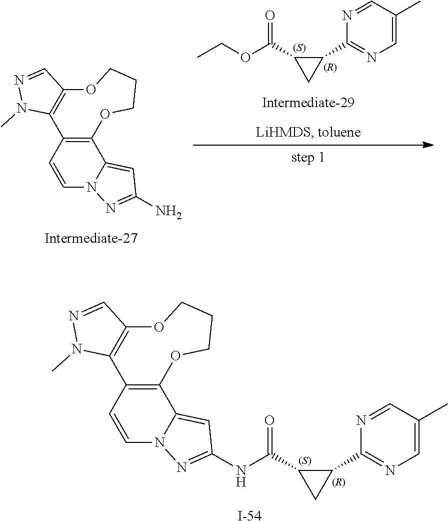

Step 1. To a solution of 11-methyl-6,7-dihydro-5H,11H-pyrazolo[1,5-a]pyrazolo[3′,4′:8,9][1,5]dioxonino[6,7-c]pyridin-2-amine (Intermediate-27) (50 mg, 175 μmol, 1.0 eq) and ethyl (1S,2R)-2-(5-methylpyrimidin-2-yl)cyclopropane-1-carboxylate (Intermediate-29) (54 mg, 263 μmol, 1.5 eq) in toluene (1 mL) was added LiHMDS (437 μL, 1M in THF, 2.5 eq) dropwise at 0° C. and the resulting mixture was stirred at 40° C. for 1 h under N₂ atmosphere. The reaction mixture was poured into saturated aqueous NH₄Cl (10 mL) and extracted with EtOAc (10 mL×3). The combined organic layers were washed with brine (10 mL), dried over anhydrous Na₂SO₄, filtered and the filtrate was concentrated under reduced pressure. The residue was purified by reverse phase HPLC (C18 column, water (0.1% FA)-ACN) to afford the title compound. LCMS: 446.1 [M+H]⁺. ¹H NMR (400 MHz, Methanol-d4) δ=8.52 (s, 2H), 8.23 (d, 1H), 7.41 (s, 1H), 6.78 (s, 1H), 6.69 (d, 1H), 4.43-4.32 (m, 2H), 4.15 (t, 2H), 3.73 (s, 3H), 2.88-2.78 (m, 1H), 2.50-2.40 (m, 1H), 2.27 (s, 3H), 2.10-2.00 (m, 3H), 1.60-1.44 (m, 1H) ppm.

### Example 12. Synthesis of (1S,2R)—N-[(9R)-3,9-dimethyl-7,10-dioxa-3,4,15,16-tetraazatetracyclo-[9.7.0.0\hat{ }{2,6}0.0\hat{ }{12,16}]octadeca-1(11),2(6),4,12,14,17-hexaen-14-yl]-2-(5-methylpyrimidin-2-yl)cyclopropane-1-carboxamide (I-64)

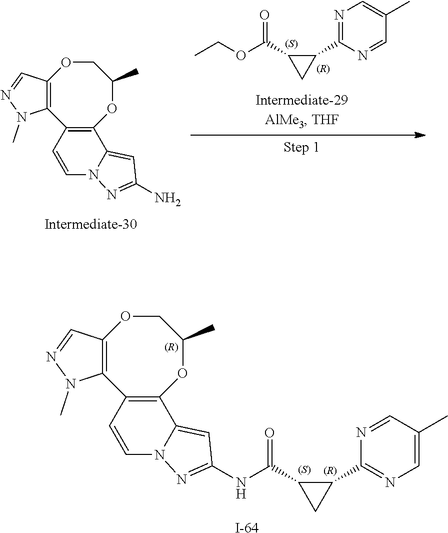

Step 1. To a solution of (R)-2,7-dimethyl-2,3-dihydro-7H-pyrazolo[1,5-a]pyrazolo[3′,4′:7,8][1,4]dioxocino[5,6-c]pyridin-12-amine (Intermediate-30) (25 mg, 53 μmol, 1.0 eq) and ethyl (1S,2R)-2-(5-methylpyrimidin-2-yl)cyclopropane-1-carboxylate (Intermediate-29) (16 mg, 79 μmol, 1.6 eq) in THF (1 mL) was added AlMe₃ (2 M in toluene, 394 μL, 15.0 eq) at 0° C. and the resulting mixture was stirred at 60° C. overnight. The reaction mixture was poured into aqueous HCl solution (1 M, 2 mL) and extracted with DCM (5 mL×2). The combined organic layers were washed with brine (5 mL), dried over anhydrous Na₂SO₄, filtered and the filtrate was concentrated under reduced pressure. The residue was purified by reverse phase HPLC (C18 column, water (0.1% FA)-ACN) to afford the title compound. LCMS: 446.2 [M+H]⁺. ¹H NMR (400 MHz, Methanol-d₄) δ=8.54 (s, 2H), 8.25 (d, 1H), 7.32 (s, 1H), 6.81 (d, 1H), 6.71 (s, 1H), 4.35-4.01 (m, 2H), 3.80 (s, 3H), 3.60-3.48 (m, 1H), 2.94-2.77 (m, 1H), 2.56-2.40 (m, 1H), 2.28 (s, 3H), 2.08-1.97 (m, 1H), 1.61-1.47 (m, 1H), 1.40 (d, 3H) ppm.

### Example 13. Synthesis of (1S,2R)-2-(5-methylpyrimidin-2-yl)-N-[(8R,9R)-3,8,9-trimethyl-7,10-dioxa-3,4,15,16-tetraazatetracyclo[9.7.0.0\hat{ }{2,6}.0\hat{ }{12,16}]octadeca-1(11),2(6),4,12,14,17-hexaen-14-yl]cyclopropane-1-carboxamide (I-116)

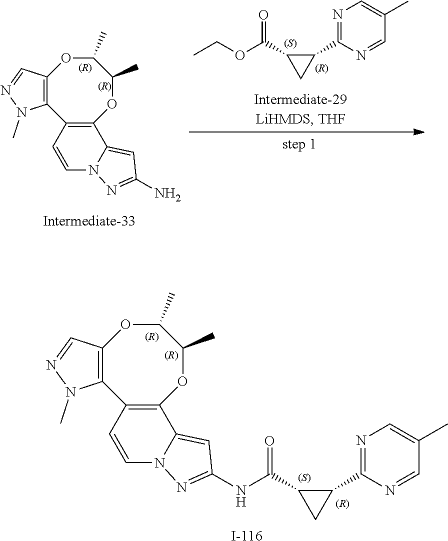

Step 1. To a solution of (2R,3R)-2,3,7-trimethyl-2,3-dihydro-7H-pyrazolo[1,5-a]pyrazolo[3′,4′:7,8][1,4]dioxocino[5,6-c]pyridin-12-amine (Intermediate-33) (30 mg, 100 μmol, 1.0 eq) and ethyl (1S,2R)-2-(5-methylpyrimidin-2-yl)cyclopropane-1-carboxylate (Intermediate-29) (31 mg, 150 μmol, 1.5 eq) in THF (1 mL) was added LiHMDS (250 μL, 1 M in THF, 2.5 eq) at 0° C. and the resulting mixture was stirred at 40° C. for 1 h under N₂ atmosphere. The reaction mixture was poured into saturated aqueous NH₄Cl (5 mL) and extracted with EtOAc (5 mL×3). The combined organic layers were washed with brine (3 mL), dried over anhydrous Na₂SO₄, filtered and the filtrate was concentrated under reduced pressure. The residue was purified by flash silica gel chromatography (eluent of EtOAc/MeOH) to afford the title compound. LCMS: 460.2 [M+H]⁺. ¹H NMR (400 MHz, DMSO-d6) δ 11.09 (s, 1H), 8.55-8.39 (m, 3H), 7.39 (s, 1H), 6.96 (d, 1H), 6.67 (s, 1H), 3.94-3.77 (m, 4H), 3.68-3.54 (m, 1H), 2.78-2.66 (m, 1H), 2.45-2.38 (m, 1H), 2.19 (s, 3H), 1.92-1.78 (m, 1H), 1.43-1.32 (m, 4H), 1.23 (d, 3H) ppm.

### Example 14. Synthesis of (1S,2R)-2-[5-(difluoromethyl)pyrimidin-2-yl]-N-[(8R,9R)-3,8,9-trimethyl-7,10-dioxa-3,4,15,16-tetraazatetracyclo[9.7.0.0\hat{ }{2,6}.0\hat{ }{12,16}]octadeca-1(11),2(6),4,12,14,17-hexaen-14-yl]cyclopropane-1-carboxamide (I-117)

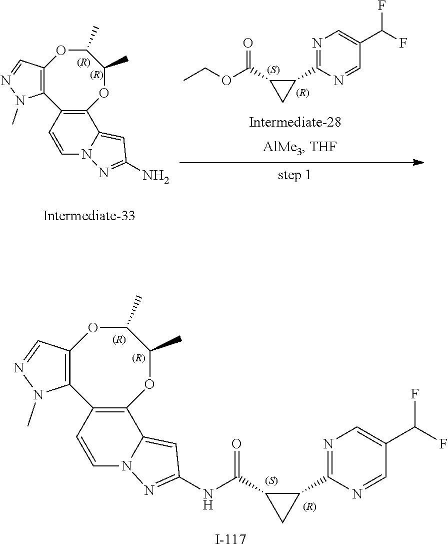

Step 1. To a solution of (2R,3R)-2,3,7-trimethyl-2,3-dihydro-7H-pyrazolo[1,5-a]pyrazolo[3′,4′:7,8][1,4]dioxocino[5,6-c]pyridin-12-amine (Intermediate-33) (100 mg, 334 μmol, 1.0 eq) and ethyl (1S,2R)-2-(5-(difluoromethyl)pyrimidin-2-yl)cyclopropane-1-carboxylate (Intermediate-28) (121 mg, 501 μmol, 1.5 eq) in THF (2 mL) was added AlMe₃ (668 μL, 2 M in toluene, 4.0 eq) and the resulting mixture was stirred at 60° C. for 3 h under N₂ atmosphere. The reaction mixture was poured into aqueous HCl (1 N, 5 mL) and extracted with EtOAc (10 mL×3). The combined organic layers were washed with brine (10 mL), dried over anhydrous Na₂SO₄, filtered and the filtrate was concentrated under reduced pressure. The residue was purified by flash silica gel chromatography (eluent of EtOAc/MeOH) to afford the title compound. LCMS: 496.1 [M+H]⁺. ¹H NMR (400 MHz, DMSO-d6) δ=11.17 (s, 1H), 8.89 (s, 2H), 8.45 (d, 1H), 7.39 (s, 1H), 7.30-7.00 (m, 1H), 6.96 (d, 1H), 6.64 (s, 1H), 3.92-3.73 (m, 4H), 3.67-3.54 (m, 1H), 2.94-2.82 (m, 1H), 2.56-2.51 (m, 1H), 1.97-1.85 (m, 1H), 1.52-1.43 (m, 1H), 1.34 (d, 3H), 1.23 (d, 3H) ppm.

### Example 15. Synthesis of 3-[(9R)-3,9-dimethyl-7,10-dioxa-3,4,15,16-tetraazatetracyclo-[9.7.0.0\hat{ }{2,6}0.0\hat{ }{12,16}]octadeca-1(11),2(6),4,12,14,17-hexaen-14-yl]-1-[(1R*)-1-(5-methylpyrimidin-2-yl)ethyl]urea (stereoisomer 1) (I-142) and 3-[(9R)-3,9-dimethyl-7,10-dioxa-3,4,15,16-tetraazatetracyclo-[9.7.0.0\hat{ }{2,6}.0\hat{ }{12,16}]octadeca-1(11),2(6),4,12,14,17-hexaen-14-yl]-1-[(1R*)-1-(5-methylpyrimidin-2-yl)ethyl]urea (stereoisomer 2) (I-143)

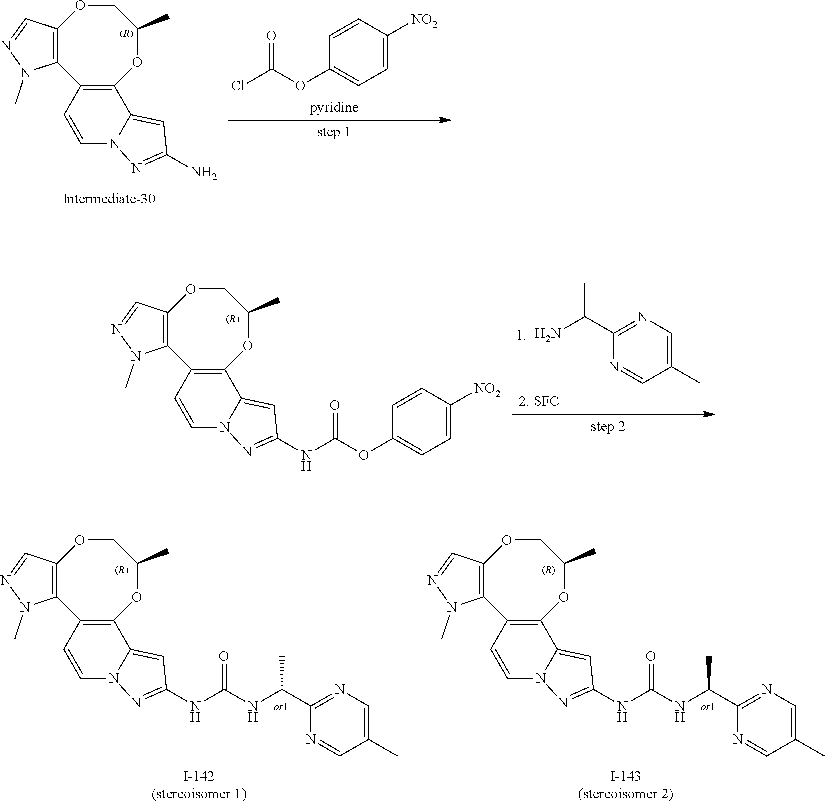

Step 1. Synthesis of 4-nitrophenyl (R)-(2,7-dimethyl-2,3-dihydro-7H-pyrazolo[1,5-a]pyrazolo[3′,4′:7,8][1,4]dioxocino[5,6-c]pyridin-12-yl)carbamate. To a solution of (R)-2,7-dimethyl-2,3-dihydro-7H-pyrazolo[1,5-a]pyrazolo[3′,4′:7,8][1,4]dioxocino[5,6-c]pyridin-12-amine (Intermediate-30) (100 mg, 351 μmol, 1.0 eq) in THF (2 mL) was added pyridine (33 mg, 421 μmol, 1.2 eq) and 4-nitrophenyl carbonochloridate (78 mg, 386 μmol, 1.1 eq) at 0° C., and then stirred at room temperature for 1 h to afford the title compound, the reaction solution was used into the next step without further workup and purification.

Step 2. Synthesis of 3-[(9R)-3,9-dimethyl-7,10-dioxa-3,4,15,16-tetraazatetracyclo-[9.7.0.0\hat{ }{2,6}0.0\hat{ }{12,16}]octadeca-1(11),2(6),4,12,14,17-hexaen-14-yl]-1-[(1R*)-1-(5-methylpyrimidin-2-yl)ethyl]urea (stereoisomer 1) and 3-[(9R)-3,9-dimethyl-7,10-dioxa-3,4,15,16-tetraazatetracyclo-[9.7.0.0\hat{ }{2,6}0.0\hat{ }{12,16}]octadeca-1(11),2(6),4,12,14,17-hexaen-14-yl]-1-[(1R*)-1-(5-methylpyrimidin-2-yl)ethyl]urea (stereoisomer 2). To the reaction mixture of step 1 was added 1-(5-methylpyrimidin-2-yl)ethan-1-amine (racemic) (183 mg, 1.33 mmol, 4.0 eq) and the resulting mixture was stirred at room temperature for 1 h. The reaction mixture was concentrated under reduced pressure and then purified by reverse phase HPLC (C18 column, water (0.1% FA)-ACN) to afford the mixture of title compounds. LCMS: 449.2 [M+H]⁺.

The resulting mixture of title compounds (80 mg, 178 μmol, 1.0 eq) was separated by chiral SFC (Instrument: Waters 80Q Preparative SFC system; Column: DAICEL Chiralpak AD 250×25 mm I.D., 10 μm particle size; Mobile Phase: Phase A for Supercritical CO₂, Phase B for EtOH+ACN (0.2% NH₃·H₂O); Isocratic elution: 55% Phase B in Supercritical CO₂; Flow rate: 70 g/min; Retention Time: Peak1/3.88 min, Peak2/5.38 min; Back Pressure: 100 bar to keep the CO₂ in Supercritical flow; UV: 220 nm).

The first elution peak (Peak1/3.88 min): 3-[(9R)-3,9-dimethyl-7,10-dioxa-3,4,15,16-tetraazatetracyclo-[9.7.0.0\hat{ }{2,6}0.0\hat{ }{12,16}]octadeca-1(11),2(6),4,12,14,17-hexaen-14-yl]-1-[(1R*)-1-(5-methylpyrimidin-2-yl)ethyl]urea (stereoisomer 1) (I-142). LCMS: MS: 449.2 [M+H]⁺. ¹H NMR (400 MHz, Methanol-d₄) δ=8.64 (s, 2H), 8.32 (d, 1H), 7.35 (s, 1H), 6.83 (d, 1H), 6.60 (s, 1H), 5.11 (d, 1H), 4.25 (br d, 2H), 3.86 (s, 3H), 3.67-3.51 (m, 1H), 2.34 (s, 3H), 1.56 (d, 3H), 1.47 (d, 3H) ppm.

The second elution peak (Peak2/5.38 min): 3-[(9R)-3,9-dimethyl-7,10-dioxa-3,4,15,16-tetraazatetracyclo-[9.7.0.0\hat{ }{2,6}0.0\hat{ }{12,16}]octadeca-1(11),2(6),4,12,14,17-hexaen-14-yl]-1-[(1R*)-1-(5-methylpyrimidin-2-yl)ethyl]urea (stereoisomer 2) (I-143). LCMS: 449.2 [M+H]⁺. ¹H NMR (400 MHz, Methanol-d₄) δ=8.64 (s, 2H), 8.31 (d, 1H), 7.35 (s, 1H), 6.83 (d, 1H), 6.60 (s, 1H), 5.17-5.06 (m, 1H), 4.24 (br d, 2H), 3.87 (s, 3H), 3.60 (br d, 1H), 2.33 (s, 3H), 1.56 (d, 3H), 1.47 (d, 3H) ppm.

### Example 16. Synthesis of (1S,2R)—N-[(9R)-3,9-dimethyl-7,10-dioxa-3,4,12,15-tetraazatetracyclo[9.7.0.0\hat{ }{2,6}0.0\hat{ }{12,16}]octadeca-1(11),2(6),4,13,15,17-hexaen-14-yl]-2-(5-methylpyrimidin-2-yl)cyclopropane-1-carboxamide (I-340)

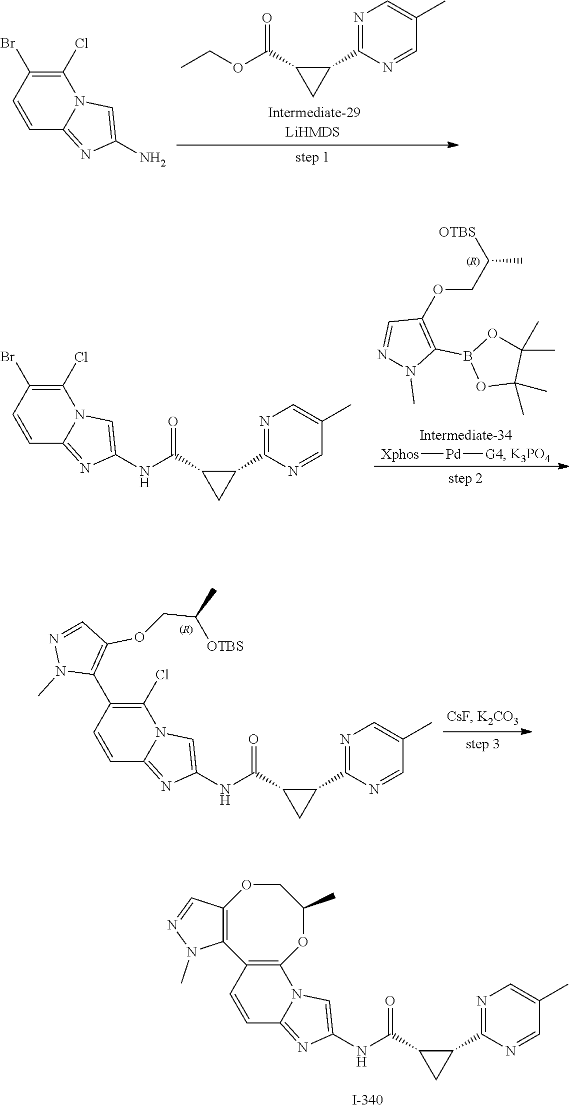

Step 1. Synthesis of (1S,2R)—N-(6-bromo-5-chloroimidazo[1,2-a]pyridin-2-yl)-2-(5-methylpyrimidin-2-yl)cyclopropane-1-carboxamide. To a solution of 6-bromo-5-chloroimidazo[1,2-a]pyridin-2-amine (1.00 g, 4.06 mmol, 1.0 eq) and ethyl (1S,2R)-2-(5-methylpyrimidin-2-yl)cyclopropane-1-carboxylate (Intermediate-29) (1.09 g, 5.27 mmol, 1.3 eq) in THF (10 mL) was added LiHMDS (1 M in THF, 10.14 mL, 2.5 eq) at 0° C., then the resulting mixture was stirred at 40° C. for 1 h under N₂ atmosphere. The reaction mixture was slowly poured into saturated aqueous NH₄Cl (100 mL) and then extracted with EtOAc (50 mL×2). The combined organic layers were washed with brine (40 mL), dried over anhydrous Na₂SO₄, filtered and concentrated under reduced pressure. The residue was purified by flash silica gel chromatography (eluent of MeOH/DCM) to afford the title compound. LCMS: 408.0 [M+H]⁺.

Step 2. Synthesis of (1S,2R)—N-(6-(4-((R)-2-((tert-butyldimethylsilyl)oxy)propoxy)-1-methyl-1H-pyrazol-5-yl)-5-chloroimidazo[1,2-a]pyridin-2-yl)-2-(5-methylpyrimidin-2-yl)cyclopropane-1-carboxamide. To a solution of (1S,2R)—N-(6-bromo-5-chloroimidazo[1,2-a]pyridin-2-yl)-2-(5-methylpyrimidin-2-yl)cyclopropane-1-carboxamide (400 mg, 984 μmol, 1.0 eq) and (R)-4-(2-((tert-butyldimethylsilyl)oxy)propoxy)-1-methyl-5-(4,4,5,5-tetramethyl-1,3,2-dioxaborolan-2-yl)-1H-pyrazole (Intermediate-34) (507 mg, 1.28 mmol, 1.3 eq) in 1,4-dioxane (6 mL) and H₂O (1 mL) was added Xphos Pd G₄ (169 mg, 197 μmol, 0.2 eq) and K₃PO₄ (679.53 mg, 2.95 mmol, 3.0 eq), then the reaction mixture was stirred at 60° C. for 2 h under N₂ atmosphere. The reaction mixture was poured into water (30 mL) and then extracted with EtOAc (15 mL×2). The combined organic layers were washed with brine (20 mL), dried over anhydrous Na₂SO₄, filtered and concentrated under reduced pressure. The residue was purified by flash silica gel chromatography (eluent of MeOH/DCM) to afford the title compound. LCMS: 596.2 [M+H]⁺.

Step 3. Synthesis of (1S,2R)—N-[(9R)-3,9-dimethyl-7,10-dioxa-3,4,12,15-tetraazatetracyclo[9.7.0.0\hat{ }{2,6}.0\hat{ }{12,16}]octadeca-1(11),2(6),4,13,15,17-hexaen-14-yl]-2-(5-methylpyrimidin-2-yl)cyclopropane-1-carboxamide. To a solution of (1S,2R)—N-(6-(4-((R)-2-((tert-butyldimethylsilyl)oxy)propoxy)-1-methyl-1H-pyrazol-5-yl)-5-chloroimidazo[1,2-a]pyridin-2-yl)-2-(5-methylpyrimidin-2-yl)cyclopropane-1-carboxamide (400 mg, 671 μmol, 1.0 eq) in DMF (5 mL) was added CsF (1.02 g, 6.71 mmol, 10.0 eq) and the resulting mixture was stirred at 100° C. for 1 h. Then K₂CO₃ (184 mg, 1.33 mmol, 2.0 eq) was added, and the resulting mixture was stirred at 100° C. overnight. The mixture was filtered, the filtrate was poured into water (50 mL). The mixture was extracted with EtOAc (30 mL×3). The combined organic layers were washed with brine (30 mL), dried over anhydrous Na₂SO₄, filtered and concentrated under reduced pressure. The residue was purified by reverse phase HPLC (C18 column, water (0.1% FA)-ACN) to afford the title compound. LCMS: 446.2 [M+H]⁺. ¹H NMR (400 MHz, Methanol-d₄) δ=8.52 (s, 2H), 7.86 (s, 1H), 7.46-7.33 (m, 3H), 4.57-4.32 (m, 1H), 4.31-4.16 (m, 1H), 3.84 (s, 3H), 3.76-3.58 (m, 1H), 2.91-2.79 (m, 1H), 2.56-2.43 (m, 1H), 2.27 (s, 3H), 2.07-2.02 (m, 1H), 1.60-1.54 (m, 1H), 1.53-1.39 (m, 3H).

### Example 17. Synthesis of 2-[5-(2-hydroxypropan-2-yl)pyridin-2-yl]-N-[(8R,9R)-3,8,9-trimethyl-7,10-dioxa-3,4,15,16-tetraazatetracyclo[9.7.0.0\hat{ }{2,6}.0\hat{ }{12,16}]octadeca-1(11),2(6),4,12,14,17-hexaen-14-yl]acetamide (I-356)

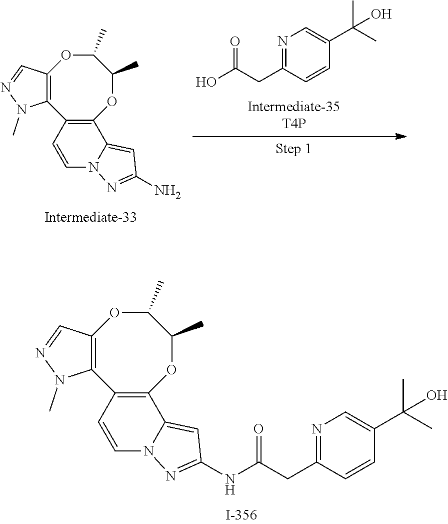 [SMILES: C[C@H]1Oc2cnn(C)c2-c2ccn3nc(N)cc3c2O[C@@H]1C ; CC(C)(O)c1ccc(CC(=O)O)nc1 ; C[C@H]1Oc2cnn(C)c2-c2ccn3nc(NC(=O)Cc4ccc(C(C)(C)O)cn4)cc3c2O[C@@H]1C]

Step 1. To a solution of 2-(5-(2-hydroxypropan-2-yl)pyridin-2-yl)acetic acid (Intermediate-35) (63 mg, 323 μmol, 1.0 eq) and (2R,3R)-2,3,7-trimethyl-2,3-dihydro-7H-pyrazolo[1,5-a]pyrazolo[3′,4′:7,8][1,4]dioxocino[5,6-c]pyridin-12-amine (Intermediate-33) (97 mg, 323 μmol, 1.0 eq) in DCM (1 mL) was added T4P (50% in EtOAc, 698 mg, 968 μmol, 3.0 eq) and pyridine (204 mg, 2.58 mmol, 8.0 eq), and the resulting mixture was stirred at room temperature for 1 h. The reaction mixture was quenched with H₂O (20 mL) and then extracted with EtOAc (20 mL×3). The combined organic layers were washed with brine (20 mL), filtered and the filtrate was concentrated under reduced pressure. The residue was purified by column chromatography (SiO₂, PE:EtOAc=50/1-0/1 to 10% MeOH in EtOAc) to give a crude. The crude was purified by reverse phase HPLC (C18 column, water (0.1% FA)-ACN) to afford the title compound. LCMS: 477.3 [M+1]⁺. ¹H NMR: (400 MHz, CDCl₃) δ=10.62 (s, 1H), 8.80 (d, 1H), 8.18 (d, 1H), 7.90-7.80 (m, 1H), 7.40 (s, 1H), 7.29 (d, 1H), 7.11 (s, 1H), 6.63 (d, 1H), 4.05-3.85 (m, 6H), 3.78-3.61 (m, 1H), 1.63 (s, 6H), 1.54 (d, 3H), 1.33 (d, 3H).

### Example 18. Synthesis of 2-{5-[(2R*)-2-methyl-1,4-dioxan-2-yl]pyridin-2-yl}-N-[(8R,9R)-3,8,9-trimethyl-7,10-dioxa-3,4,15,16-tetraazatetracyclo[9.7.0.0\hat{ }{2,6}0.0\hat{ }{12,16}]octadeca-1(11),2(6),4,12,14,17-hexaen-14-yl]acetamide (stereoisomer 1) (I-404) and 2-{5-[(2R*)-2-methyl-1,4-dioxan-2-yl]pyridin-2-yl}-N-[(8R,9R)-3,8,9-trimethyl-7,10-dioxa-3,4,15,16-tetraazatetracyclo[9.7.0.0\hat{ }{2,6}.0\hat{ }{12,16}]octadeca-1(11),2(6),4,12,14,17-hexaen-14-yl]acetamide (stereoisomer 2) (I-403)

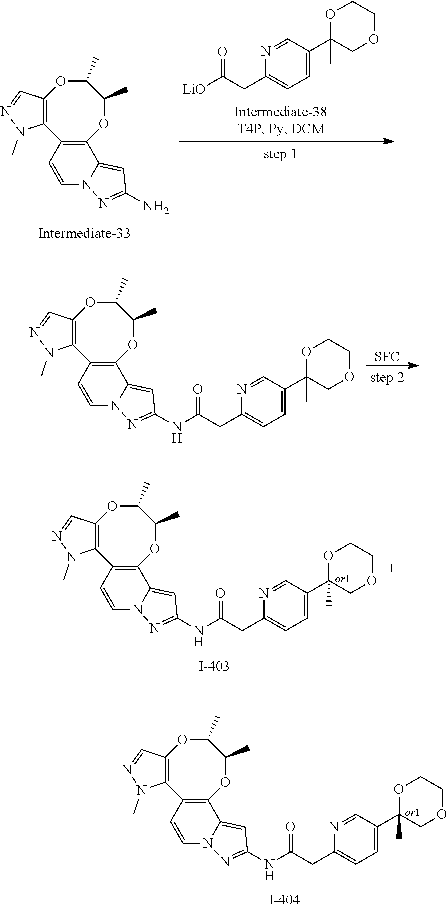

Step 1. Synthesis of 2-(5-(2-methyl-1,4-dioxan-2-yl) pyridin-2-yl)-N-((2R,3R)-2,3,7-trimethyl-2,3-dihydro-7H-pyrazolo[1,5-a]pyrazolo[3′,4′:7,8][1,4]dioxocino[5,6-c]pyridin-12-yl) acetamide. To a solution of (2R,3R)-2,3,7-trimethyl-2,3-dihydro-7H-pyrazolo[1,5-a]pyrazolo[3′,4′:7,8][1,4]dioxocino[5,6-c]pyridin-12-amine (Intermediate-33) (100 mg, 334 μmol, 1 eq) in DCM (2 mL) was added 2-(5-(2-methyl-1,4-dioxan-2-yl) pyridin-2-yl) acetic acid lithium salt (Intermediate-38) (0.15 g, 501 μmol, 1.5 eq), T₄P (0.96 g, 1.34 mmol, 50% purity, 4 eq) and pyridine (0.13 g, 1.67 mmol, 0.13 mL, 5 eq). The mixture was stirred at 25° C. for 1 h. The reaction mixture was concentrated under reduced pressure to give a residue. The residue was purified by flash silica gel chromatography (eluent of DCM/MeOH) and reverse phase preparative HPLC (ACN/H₂O, 0.1% NH₃·H₂O) to afford the title compound. LCMS: 519.2 [M+H]⁺.

Step 2. Chiral separation of 2-(5-(2-methyl-1,4-dioxan-2-yl) pyridin-2-yl)-N-((2R,3R)-2,3,7-trimethyl-2,3-dihydro-7H-pyrazolo[1,5-a]pyrazolo[3′,4′:7,8][1,4]dioxocino[5,6-c]pyridin-12-yl) acetamide. The mixture of stereoisomers obtained in step 1 was separated by chiral SFC (SFC preparative method: instrument: Waters 150 Preparative SFC system; column: DAICEL DAICEL CHIRALCEL OX 250 mm*30 mm, 10 μm; mobile phase A: CO₂; mobile phase B: EtOH:ACN=4:1 (0.1% NH₃H₂O); isocratic elution: 35% phase B in supercritical CO₂; flow rate: 150 g/min; retention time: Peak1: 6.43 min, Peak2: 7.72 min; back pressure: 100 bar; wave length: 220 nm) to give peak1 (I-404) and peak2. Peak2 was further purified by reverse phase preparative HPLC (ACN/H₂O, 0.225% FA) to afford (I-403). Analytical data for 2-{5-[(2R*)-2-methyl-1,4-dioxan-2-yl]pyridin-2-yl}-N-[(8R,9R)-3,8,9-trimethyl-7,10-dioxa-3,4,15,16-tetraazatetracyclo[9.7.0.0\hat{ }{2,6}0.0\hat{ }{12,16}]octadeca-1(11),2(6),4,12,14,17-hexaen-14-yl]acetamide (stereoisomer 1) (I-404):

¹H NMR (400 MHz, CD₃OD) δ=8.64 (d, 1H), 8.34 (d, 1H), 7.97-7.89 (m, 1H), 7.50 (d, 1H), 7.40 (s, 1H), 7.04 (s, 1H), 6.90 (d, 1H), 4.18 (d, 1H), 4.02 (s, 2H), 3.96-3.87 (m, 4H), 3.74-3.58 (m, 6H), 1.50 (d, 3H), 1.44 (s, 3H), 1.32 (d, 3H). LCMS: 519.2 [M+H]⁺. Chiral analytical SFC: Rt=1.678 min. Analytical data for 2-{5-[(2R*)-2-methyl-1,4-dioxan-2-yl]pyridin-2-yl}-N-[(8R,9R)-3,8,9-trimethyl-7,10-dioxa-3,4,15,16-tetraazatetracyclo[9.7.0.0\hat{ }{2,6}0.0\hat{ }{12,16}]octadeca-1(11),2(6),4,12,14,17-hexaen-14-yl]acetamide (stereoisomer 2) (I-403):

¹H NMR (400 MHz, CD₃OD) δ=8.64 (d, 1H), 8.34 (d, 1H), 8.00-7.86 (m, 1H), 7.50 (d, 1H), 7.40 (s, 1H), 7.04 (s, 1H), 6.90 (d, 1H), 4.18 (d, 1H), 4.02 (s, 2H), 3.97-3.85 (m, 4H), 3.76-3.57 (m, 6H), 1.50 (d, 3H), 1.44 (s, 3H), 1.32 (d, 3H). LCMS: 519.2 [M+H]⁺. Chiral analytical SFC: Rt=1.771 min

### Chiral SFC Analytical Method

| Method | OX-3-EtOH + ACN(DEA)-20-60-3ML-35T | | | |
|---|---|---|---|---|
| Instrument | SHIMADZU LC-30ADsf | | | |
| column | Chiralcel OX-3 50 × 4.6 mm I.D., particle size 3 um | | | |
| Mobile Phase | Phase A for CO₂ | | | Phase B for EtOH + ACN (0.05% DEA) |
| | Time(min) | A(%) | B(%) | |
| Gradient | 0.00 | 80 | 20 | |
| | 1.80 | 40 | 60 | |
| | 2.70 | 40 | 60 | |
| | 2.71 | 80 | 20 | |
| | 3.00 | 80 | 20 | |

| | | |
|---|---|---|
| Flow rate | 3.0 | mL/min |
| Column Temp | 35° | C. |
| Back pressure | 10 | MPa |
| UV | 220 | nm |

NOTE:
EtOH:ACN = 2:1

### Example 19. Synthesis of (1S,2R)-2-(5-methylpyrimidin-2-yl)-N-[(8R*,9R*)-3,8,9-trimethyl-7,10-dioxa-3,4,12,15-tetraazatetracyclo[9.7.0.0\hat{ }{2,6}.0\hat{ }{12,16}]octadeca-1(11),2(6),4,13,15,17-hexaen-14-yl]cyclopropane-1-carboxamide (stereoisomer 2) (I-227) and (1S,2R)-2-(5-methylpyrimidin-2-yl)-N-[(8R*,9R*)-3,8,9-trimethyl-7,10-dioxa-3,4,12,15-tetraazatetracyclo[9.7.0.0\hat{ }{2,6}.0\hat{ }{12,16}]octadeca-1(11),2(6),4,13,15,17-hexaen-14-yl]cyclopropane-1-carboxamide (stereoisomer 1) (I-226)

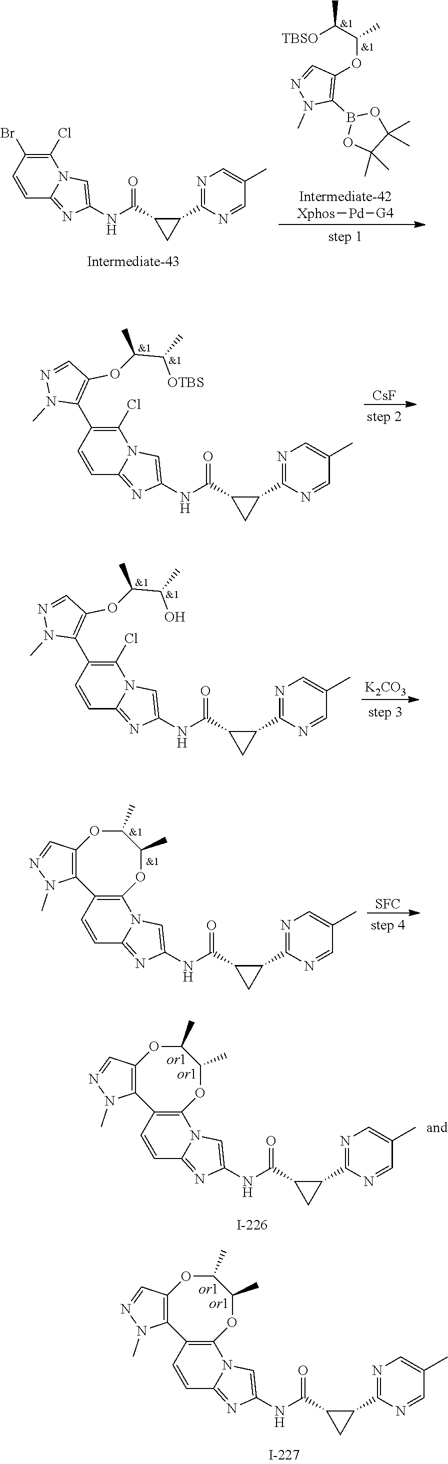

Step 1. Synthesis of (1S,2R)—N-(6-(4-(((2RS,3RS)-3-((tert-butyldimethylsilyl)oxy)butan-2-yl)oxy)-1-methyl-1H-pyrazol-5-yl)-5-chloroimidazo[1,2-a]pyridin-2-yl)-2-(5-methylpyrimidin-2-yl)cyclopropane-1-carboxamide. To a solution of (1S, 2R)—N-(6-bromo-5-chloro-imidazo[1, 2-a]pyridin-2-yl)-2-(5-methylpyrimidin-2-yl) cyclopropanecarboxamide (Intermediate-43) (5.00 g, 12.30 mmol, 1.0 eq) and rac-4-(((2R,3R)-3-((tert-butyldimethylsilyl)oxy)butan-2-yl)oxy)-1-methyl-5-(4,4,5,5-tetramethyl-1,3,2-dioxaborolan-2-yl)-1H-pyrazole (Intermediate-42) (6.56 g, 15.98 mmol, 1.3 eq) in 1,4-dioxane (50 mL) and H₂O (5 mL) was added K₃PO₄ (7.83 g, 36.89 mmol, 3.0 eq) and Xphos-Pd-G4 (2.12 g, 2.46 mmol, 0.2 eq) under N₂ atmosphere and the reaction mixture was stirred at 60° C. under N₂ for 1.5 h. The reaction mixture was filtered and the filtrate was concentrated under reduced pressure to give a residue. The residue was purified by flash silica gel chromatography (eluent of EtOAc/PE) to afford the title compound. LCMS: 610.5 [M+H]⁺.

Step 2. Synthesis of (1S,2R)—N-(5-chloro-6-(4-(((2RS,3RS)-3-hydroxybutan-2-yl)oxy)-1-methyl-1H-pyrazol-5-yl)imidazo[1,2-a]pyridin-2-yl)-2-(5-methylpyrimidin-2-yl)cyclopropane-1-carboxamide. To a solution of (1S,2R)—N-(6-(4-(((2RS,3RS)-3-((tert-butyldimethylsilyl)oxy)butan-2-yl)oxy)-1-methyl-1H-pyrazol-5-yl)-5-chloroimidazo[1,2-a]pyridin-2-yl)-2-(5-methylpyrimidin-2-yl)cyclopropane-1-carboxamide (1.00 g, 1.64 mmol, 1.0 eq) in DMA (40 mL) was added CsF (1.99 g, 13.11 mmol, 8.0 eq), and the resulting mixture was stirred at 100° C. under N₂ atmosphere for 1.5 h. The reaction mixture was used in the next step without further workup and purification. LCMS: 496.2 [M+H]⁺.

Step 3. Synthesis of (1S,2R)-2-(5-methylpyrimidin-2-yl)-N-((6RS,7RS)-6,7,11-trimethyl-6,7-dihydro-11H-imidazo[1,2-a]pyrazolo[3′,4′:7,8][1,4]dioxocino[6,5-e]pyridin-2-yl)cyclopropane-1-carboxamide. To the reaction mixture of step 2 was added K₂CO₃ (446 mg, 3.23 mmol, 2.0 eq), and the resulting mixture was stirred at 100° C. under N₂ overnight. The reaction mixture was concentrated under reduced pressure and then purified by reverse phase HPLC (water (0.1% FA) ACN) to afford the title compound. LCMS: 460.2 [M+H]⁺.

Step 4. Separation of (1S,2R)-2-(5-methylpyrimidin-2-yl)-N-[(8R*,9R*)-3,8,9-trimethyl-7,10-dioxa-3,4,12,15-tetraazatetracyclo[9.7.0.0\hat{ }{2,6}0.0\hat{ }{12,16}]octadeca-1(11),2(6),4,13,15,17-hexaen-14-yl]cyclopropane-1-carboxamide (stereoisomer 1) and (1S,2R)-2-(5-methylpyrimidin-2-yl)-N-[(8R*,9R*)-3,8,9-trimethyl-7,10-dioxa-3,4,12,15-tetraazatetracyclo[9.7.0.0\hat{ }{2,6}0.0\hat{ }{12,16}]octadeca-1(11),2(6),4,13,15,17-hexaen-14-yl]cyclopropane-1-carboxamide (stereoisomer 2).

The (1S,2R)-2-(5-methylpyrimidin-2-yl)-N-((6RS,7RS)-6,7,11-trimethyl-6,7-dihydro-11H-imidazo[1,2-a]pyrazolo[3′,4′:7,8][1,4]dioxocino[6,5-e]pyridin-2-yl)cyclopropane-1-carboxamide obtained in step 3 (1.00 g, 2.18 mmol) was separated by chiral SFC (Instrument: Waters 80 Preparative SFC system; column: Daicel Chiralpak OD 250×30 mm I.D., 10 μm particle size; mobile phase A: CO₂, mobile phase B: IPA+ACN (0.1% NH₃H₂O); isocratic elution: 35% phase B in supercritical CO₂; flow rate: 150 g/min; retention time: peak1—3.33 min, Peak2-4.27 min; back pressure: 100 bar; wave length: 220 nm) to afford the tittle compounds.

(1S,2R)-2-(5-methylpyrimidin-2-yl)-N-[(8R*,9R*)-3,8,9-trimethyl-7,10-dioxa-3,4,12,15-tetraazatetracyclo[9.7.0.0\hat{ }{2,6}0.0\hat{ }{12,16}]octadeca-1(11),2(6),4,13,15,17-hexaen-14-yl]cyclopropane-1-carboxamide, first eluting isomer as stereoisomer 1) (I-226), chiral SFC retention time: 3.33 minutes. LCMS: 460.2 [M+H]⁺. ¹H NMR (400 MHz, CD₃OD) δ 8.54 (s, 2H), 7.88 (d, 1H), 7.48-7.35 (m, 3H), 4.26-4.05 (m, 1H), 3.89 (d, 3H), 3.83-3.69 (m, 1H), 2.92-2.80 (m, 1H), 2.58-2.44 (m, 1H), 2.30 (s, 3H), 2.11-2.03 (m, 1H), 1.64-1.48 (m, 4H), 1.40-1.30 (m, 3H).

(1S,2R)-2-(5-methylpyrimidin-2-yl)-N-[(8R*,9R*)-3,8,9-trimethyl-7,10-dioxa-3,4,12,15-tetraazatetracyclo[9.7.0.0\hat{ }{2,6}0.0\hat{ }{12,16}]octadeca-1(11),2(6),4,13,15,17-hexaen-14-yl]cyclopropane-1-carboxamide, second eluting stereoisomer as stereoisomer 2 (I-227), chiral SFC retention time: 4.27 minutes LCMS: 460.2 [M+H]⁺. ¹H NMR (400 MHz, CD₃OD) δ 8.52 (s, 2H), 7.85 (s, 1H), 7.44-7.40 (m, 1H), 7.39 (s, 1H), 7.37-7.32 (m, 1H), 4.13 (br s, 1H), 3.86 (s, 3H), 3.79-3.68 (m, 1H), 2.89-2.79 (m, 1H), 2.55-2.46 (m, 1H), 2.26 (s, 3H), 2.08-1.99 (m, 1H), 1.59-1.44 (m, 4H), 1.32 (d, 3H).

### Example 20. Synthesis of (1R*)-1-(5-methylpyrimidin-2-yl)ethyl N-[(8R,9R)-3,8,9-trimethyl-7,10-dioxa-3,4,15,16-tetraazatetracyclo[9.7.0.0\hat{ }{2,6}.0\hat{ }{12,16}]octadeca-1(11),2(6),4,12,14,17-hexaen-14-yl]carbamate (stereoisomer 2) (I-268)

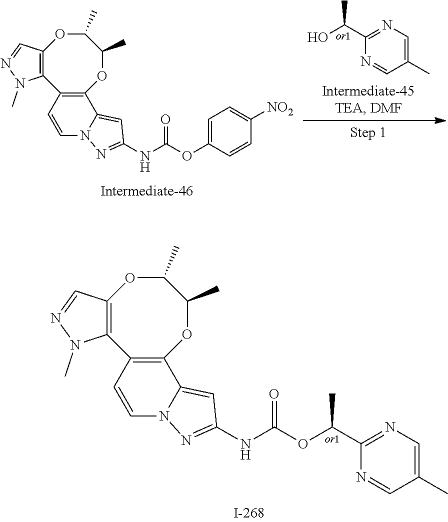

Step 1. To a solution of rel-(R)-1-(5-methylpyrimidin-2-yl)ethan-1-ol (stereoisomer 2, Intermediate-45) (20 mg, 145 μmol, 1.0 eq) in DMF (1 mL) was added TEA (18 mg, 174 μmol, 1.2 eq), and the resulting mixture was stirred at 30° C. for 0.5 h. Then 4-nitrophenyl ((2R,3R)-2,3,7-trimethyl-2,3-dihydro-7H-pyrazolo[1,5-a]pyrazolo[3′,4′:7,8][1,4]dioxocino[5,6-c]pyridin-12-yl)carbamate (Intermediate-45) (81 mg, 1734 μmol, 1.2 eq) was added slowly, and the resulting mixture was stirred at 60° C. for 10 min. The reaction mixture was poured into H₂O (10 mL), and extracted with DCM (10 mL×2). The combined organic layers were washed with brine (10 mL), dried over anhydrous Na₂SO₄, filtered and the filtrate was concentrated under reduced pressure. The residue was purified by reverse phase HPLC (C18 column, water (0.1% FA)-ACN) to afford the title compound. LCMS: 464.2 [M+H]⁺. ¹H NMR (400 MHz, CDCl₃) δ 8.66 (br s, 1H), 8.60 (s, 2H), 8.40 (br d, 1H), 7.41 (s, 1H), 7.31-7.30 (m, 1H), 6.90 (s, 1H), 6.60 (d, 1H), 4.04-3.94 (m, 1H), 3.89 (s, 3H), 3.74-3.65 (m, 1H), 2.33 (s, 3H), 1.73 (br d, 3H), 1.51 (d, 3H), 1.34 (d, 3H).

### Example 21. Synthesis of (1S,2R)-2-[5-(difluoromethyl)pyridin-2-yl]-N-{3-[(1R*)-1-hydroxyethyl]-5,10-dioxa-4,15,16-triazatetracyclo[9.7.0.0\hat{ }{2,6}.0\hat{ }{12,16}]octadeca-1(11),2(6),3,12,14,17-hexaen-14-yl}cyclopropane-1-carboxamide (stereoisomer 1) (I-312) and (1S,2R)-2-[5-(difluoromethyl)pyridin-2-yl]-N-{3-[(1R*)-1-hydroxyethyl]-5,10-dioxa-4,15,16-triazatetracyclo[9.7.0.0\hat{ }{2,6}0.0\hat{ }{12,16}]octadeca-1(11),2(6),3,12,14,17-hexaen-14-yl}cyclopropane-1-carboxamide (stereoisomer 2) (I-313)

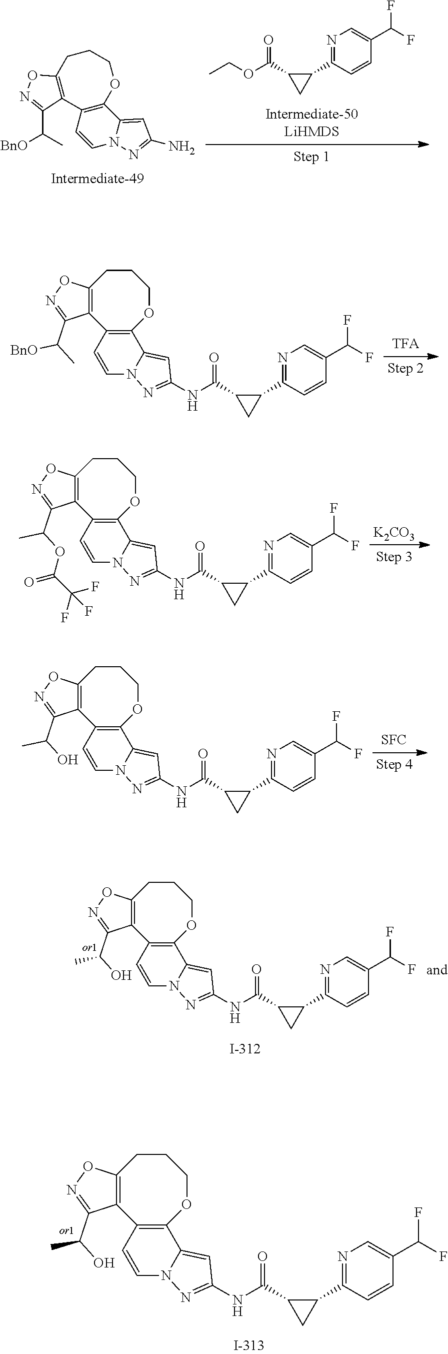

Step 1. Synthesis of (1S,2R)—N-(10-(1-(benzyloxy)ethyl)-6,7-dihydro-5H-isoxazolo[4′,5′:4,5]oxocino[2,3-c]pyrazolo[1,5-a]pyridin-2-yl)-2-(5-(difluoromethyl)pyridin-2-yl)cyclopropane-1-carboxamide. To a solution of 10-(1-(benzyloxy)ethyl)-6,7-dihydro-5H-isoxazolo[4′,5′:4,5]oxocino[2,3-c]pyrazolo[1,5-a]pyridin-2-amine (Intermediate-49) (70 mg, 179 μmol, 1.0 eq) and ethyl (1S,2R)-2-(5-(difluoromethyl)pyridin-2-yl)cyclopropane-1-carboxylate (Intermediate-50, prepared by analogy to Intermediate-28 starting from 6-bromo-3-pyridinecarboxaldehyde) (65 mg, 269 μmol, 1.5 eq) in THF (2 mL) was added LiHMDS (1 M in THF, 359 μL, 2.0 eq) at 0° C., and the resulting mixture was stirred at room temperature for 0.5 h under N₂ atmosphere. The reaction mixture was quenched with saturated aqueous NH₄Cl (1 mL), and then diluted with H₂O (10 mL) and extracted with EtOAc (10 mL×2). The combined organic layers were washed with brine (10 mL), dried with anhydrous Na₂SO₄, filtered and the filtrate was concentrated under reduced pressure. The residue was purified by flash silica gel chromatography (eluent of EtOAc/PE) to afford the title compound. LCMS: 586.1 [M+H]⁺.

Step 2. Synthesis of 1-(2-((1S,2R)-2-(5-(difluoromethyl)pyridin-2-yl)cyclopropane-1-carboxamido)-6,7-dihydro-5H-isoxazolo[4′,5′:4,5]oxocino[2,3-c]pyrazolo[1,5-a]pyridin-10-yl)ethyl 2,2,2-trifluoroacetate. To (1S,2R)—N-(10-(1-(benzyloxy)ethyl)-6,7-dihydro-5H-isoxazolo[4′,5′:4,5]oxocino[2,3-c]pyrazolo[1,5-a]pyridin-2-yl)-2-(5-(difluoromethyl)pyridin-2-yl)cyclopropane-1-carboxamide (60 mg, 102 μmol, 1.0 eq) was added TFA (1 mL) and TfOH (0.5 mL), and the resulting mixture was stirred at room temperature for 0.5 h. The reaction mixture was concentrated under reduced pressure to afford the title compound, which was used in the next step without further purification. LCMS: 592.1 [M+H]⁺.

Step 3. Synthesis of (1S,2R)-2-(5-(difluoromethyl)pyridin-2-yl)-N-(10-(1-hydroxyethyl)-6,7-dihydro-5H-isoxazolo[4′,5′:4,5]oxocino[2,3-c]pyrazolo[1,5-a]pyridin-2-yl)cyclopropane-1-carboxamide. To a solution of 1-(2-((1S,2R)-2-(5-(difluoromethyl)pyridin-2-yl)cyclopropane-1-carboxamido)-6,7-dihydro-5H-isoxazolo[4′,5′:4,5]oxocino[2,3-c]pyrazolo[1,5-a]pyridin-10-yl)ethyl 2,2,2-trifluoroacetate (60 mg, 101 μmol, 1.0 eq) in MeOH (1 mL) and H₂O (3 mL) was added K₂CO₃ (140 mg, 1.01 mmol, 10.0 eq), and the resulting mixture was stirred at room temperature for 0.5 h. The reaction mixture was diluted with water (10 mL) and then extracted with EtOAc (10 mL×3). The combined organic layers were washed with brine (10 mL), dried over anhydrous Na₂SO₄, filtered and the filtrate was concentrated under reduced pressure. The residue was purified by reverse phase HPLC (C18 column, water (0.1% FA)-ACN) to afford the title compound.

Step 4. Chiral separation of (1S,2R)-2-(5-(difluoromethyl)pyridin-2-yl)-N-(10-(1-hydroxyethyl)-6,7-dihydro-5H-isoxazolo[4′,5′:4,5]oxocino[2,3-c]pyrazolo[1,5-a]pyridin-2-yl)cyclopropane-1-carboxamide. (1S,2R)-2-(5-(difluoromethyl)pyridin-2-yl)-N-(10-(1-hydroxyethyl)-6,7-dihydro-5H-isoxazolo[4′,5′:4,5]oxocino[2,3-c]pyrazolo[1,5-a]pyridin-2-yl)cyclopropane-1-carboxamide was separated by chiral SFC (Instrument: Waters 80 Preparative SFC system; column: Daicel Chiralpak IM 250×30 mm I.D., 10 μm particle size; mobile phase A: CO₂, mobile phase B: EtOH (0.1% NH₃H₂O); isocratic elution: 55% phase B in supercritical CO₂; flow rate: 70 g/min; retention time: peak1/6.20 min, peak2/7.75 min; back pressure: 100 bar; wave length: 220 nm) to afford the title compounds.

(1S,2R)-2-(5-(difluoromethyl)pyridin-2-yl)-N-(10-((R*)-1-hydroxyethyl)-6,7-dihydro-5H-isoxazolo[4′,5′:4,5]oxocino[2,3-c]pyrazolo[1,5-a]pyridin-2-yl)cyclopropane-1-carboxamide, single isomer, first eluting isomer as stereoisomer 1 (I-312), chiral SFC retention time: 6.20 minutes. LCMS: 496.1 [M+H]⁺. ¹H NMR (400 MHz, CDCl₃) δ 9.44 (br s, 1H), 8.69 (br s, 1H), 8.12 (br d, 1H), 7.88 (br d, 1H), 7.55 (br d, 1H), 7.02 (d, 1H), 6.89 (s, 1H), 6.85-6.54 (m, 1H), 5.09-4.98 (m, 1H), 4.30 (br d, 2H), 3.12-2.92 (m, 2H), 2.90-2.80 (m, 1H), 2.51-2.34 (m, 1H), 2.13-2.08 (m, 1H), 1.93-1.89 (m, 2H), 1.71-1.66 (m, 1H), 1.52 (d, 3H).

(1S,2R)-2-(5-(difluoromethyl)pyridin-2-yl)-N-(10-((R*)-1-hydroxyethyl)-6,7-dihydro-5H-isoxazolo[4′,5′:4,5]oxocino[2,3-c]pyrazolo[1,5-a]pyridin-2-yl)cyclopropane-1-carboxamide, single isomer, second eluting isomer as stereoisomer 2 (I-313), chiral SFC retention time: 7.75 minutes. LCMS: 496.1 [M+H]⁺. ¹H NMR (400 MHz, CDCl₃) δ 9.46 (s, 1H), 8.68 (s, 1H), 8.11 (d, 1H), 7.85 (br d, 1H), 7.52 (d, 1H), 7.02 (d, 1H), 6.90 (s, 1H), 6.85-6.53 (m, 1H), 5.08-4.96 (m, 1H), 4.45-4.17 (m, 2H), 2.99 (br d, 2H), 2.88-2.77 (m, 1H), 2.45-2.35 (m, 1H), 2.14-2.06 (m, 1H), 1.96-1.90 (m, 2H), 1.70-1.66 (m, 1H), 1.53 (d, 3H).

### Example 22. Synthesis of (1S,2R)-2-[5-(difluoromethyl)pyrimidin-2-yl]-N-[(8R*,11S*)-3-methyl-7,12-dioxa-3,4,17,18-tetraazapentacyclo[11.7.0.0\hat{ }{2,6}0.0\hat{ }{8,11}0.0\hat{ }{14,18}]icosa-1(13),2(6),4,14,16,19-hexaen-16-yl]cyclopropane-1-carboxamide (stereoisomer 1) (I-325) and (1S,2R)-2-[5-(difluoromethyl)pyrimidin-2-yl]-N-[(8R*,11S*)-3-methyl-7,12-dioxa-3,4,17,18-tetraazapentacyclo[11.7.0.0\hat{ }{2,6}0.0\hat{ }{8,11}0.0\hat{ }{14,18}]icosa-1(13),2(6),4,14,16,19-hexaen-16-yl]cyclopropane-1-carboxamide (stereoisomer 2) (I-326)

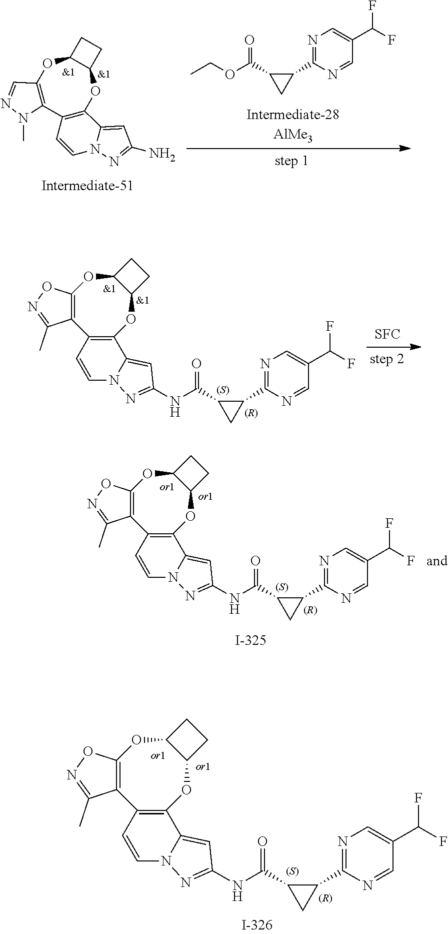

Step 1. Synthesis of (1S,2R)-2-(5-(difluoromethyl)pyrimidin-2-yl)-N-((2aRS,13aSR)-6-methyl-1,2,2a,13a-tetrahydro-6H-cyclobuta[2,3]pyrazolo[3′,4′:7,8][1,4]dioxocino[5,6-c]pyrazolo[1,5-a]pyridin-11-yl)cyclopropane-1-carboxamide. To a mixture of rac-(2aR,13aS)-6-methyl-1,2,2a,13a-tetrahydro-6H-cyclobuta[2,3]pyrazolo[3′,4′:7,8][1,4]dioxocino[5,6-c]pyrazolo[1,5-a]pyridin-11-amine (Intermediate-51) (160 mg, 538.16 μmol, 1.0 eq) and ethyl (1S,2R)-2-(5-(difluoromethyl)pyrimidin-2-yl)cyclopropane-1-carboxylate (Intermediate-28) (169.46 mg, 699.60 μmol, 1.3 eq) in THF (2 mL) was added AlMe₃ (2 μM in toluene, 1.35 mL, 5.0 eq) at 0° C. under N₂ atmosphere and the resulting mixture was stirred at 60° C. overnight. The reaction mixture was poured into aqueous HCl solution (0.5 M, 4 mL), and extracted with EtOAc (3 mL×3). The combined organic layers were washed with brine (3 mL), dried over Na₂SO₄, filtered and the filtrate was concentrated under reduced pressure. The residue was purified by flash silica gel chromatography (Eluent of MeOH/EtOAc) to afford the title compound. LCMS: 494.2 [M+H]⁺.

Step 2. Chiral separation of (1S,2R)-2-(5-(difluoromethyl)pyrimidin-2-yl)-N-((2aRS,13aSR)-6-methyl-1,2,2a,13a-tetrahydro-6H-cyclobuta[2,3]pyrazolo[3′,4′:7,8][1,4]dioxocino[5,6-c]pyrazolo[1,5-a]pyridin-11-yl)cyclopropane-1-carboxamide. (1S,2R)-2-(5-(difluoromethyl)pyrimidin-2-yl)-N-((2aRS,13aSR)-6-methyl-1,2,2a,13a-tetrahydro-6H-cyclobuta[2,3]pyrazolo[3′,4′:7,8][1,4]dioxocino[5,6-c]pyrazolo[1,5-a]pyridin-11-yl)cyclopropane-1-carboxamide was separated by chiral SFC (Instrument: Waters 150 Preparative SFC system; column: Daicel Chiralpak OX 250×30 mm I.D., 10 μm particle size; mobile phase A: CO₂, mobile phase B: IPA+ACN (0.1% NH₃H₂O); isocratic elution: 55% phase B in supercritical CO₂; flow rate: 120 g/min; retention time: peak1/2.68 min, peak2/3.53 min; back pressure: 100 bar; wave length: 220 nm) to afford the title compounds.

(1S,2R)-2-[5-(difluoromethyl)pyrimidin-2-yl]-N-[(8R*,11S*)-3-methyl-7,12-dioxa-3,4,17,18-tetraazapentacyclo[11.7.0.0\hat{ }{2,6}0.0\hat{ }{8,11}0.0\hat{ }{14,18}]icosa-1(13),2(6),4,14,16,19-hexaen-16-yl]cyclopropane-1-carboxamide, single isomer, first eluting isomer as stereoisomer 1 (I-325), chiral SFC retention time: 2.68 minutes. LCMS: 494.2 [M+H]⁺. ¹H NMR (400 MHz, DMSO-d₆) δ 11.22 (s, 1H), 8.89 (s, 2H), 8.60-8.35 (m, 1H), 7.34 (s, 1H), 7.16 (t, 1H), 7.00 (d, 1H), 6.61 (s, 1H), 4.82-4.65 (m, 1H), 4.46-4.27 (m, 1H), 3.81 (s, 3H), 2.97-2.84 (m, 1H), 2.54 (br s, 1H), 2.21-2.10 (m, 1H), 2.07-1.97 (m, 2H), 1.96-1.86 (m, 2H), 1.54-1.40 (m, 1H).

(1S,2R)-2-[5-(difluoromethyl)pyrimidin-2-yl]-N-[(8R*,11S*)-3-methyl-7,12-dioxa-3,4,17,18-tetraazapentacyclo[11.7.0.0\hat{ }{2,6}0.0\hat{ }{8,11}0.0\hat{ }{14,18}]icosa-1(13),2(6),4,14,16,19-hexaen-16-yl]cyclopropane-1-carboxamide, single isomer, second eluting isomer as stereoisomer 2 (I-326), chiral SFC retention time: 3.53 minutes. LCMS: 494.2 [M+H]⁺.

### General Synthetic Methods

All starting materials, building blocks, reagents, acids, bases, dehydrating agents, solvents, and catalysts utilized to synthesis the compounds of the present invention were either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

Proton NMR (¹H NMR) was conducted in deuterated solvent. In certain compounds disclosed herein, one or more ¹H shifts overlap with residual proteo solvent signals; these signals have not been reported in the experimental provided hereinafter.

LCMS was conduct using the following methods:

### LCMS 1

| | |
|---|---|
| Instrument | Shimadzu LCMS-2020 |
| Mode | Binary Gradient |
| Stationary Phase | HALO C18 3.0 × 30 mm, 5.0 um |
| Mobile Phase A | 0.0375% TFA in water (v/v) |
| Mobile Phase B | 0.01875% TFA in Acetonitrile (v/v) |
| Column Dimensions | 3.0 × 30 mm, 5.0 um |
| Flow Rate (mL/min) | 1.5 |
| Column Temperature (° C.) | 50 |
| Gradient | 5% to 95% B in 0.5 min, hold 95% B for 0.3 min, |
| | 95% to 5% B in 0.01 min, hold 5% B for 0.24 min |

### LCMS 2

| | |
|---|---|
| Instrument | Shimadzu LCMS-2020 |
| Mode | Binary Gradient |
| Stationary Phase | Kinetex EVO C18 2.1 × 30 mm, 5 um |
| Mobile Phase A | 0.025% NH3•H2O in water (v/v) |
| Mobile Phase B | Acetonitrile |
| Column Dimensions | 2.1 × 30 mm, 5 um |
| Flow Rate (mL/min) | 1.5 |
| Column Temperature (° C.) | 40 |
| Gradient | 5% to 95% B in 0.8 min, hold 95% B for 0.4 min, |
| | 95% to 5% B in 0.01 min, hold 5% B for 0.34 min |

TABLE 2
Selected Compounds

| | | | Mass | Retention |
|---|---|---|---|---|
| | | LCMS | Observed | time |
| I-# | Structure | Method | [g/mol] | [min] |
| I-1 | 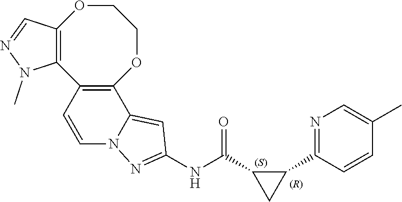 | LCMS-2 | 431.3 | 0.496 |
| I-2 | 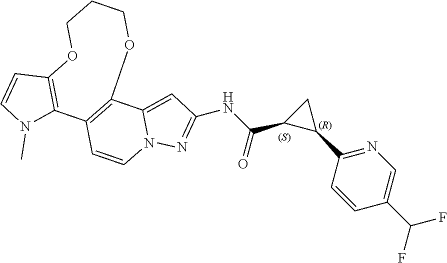 | LCMS 1 | 481.3 | 0.447 |
| I-3 | 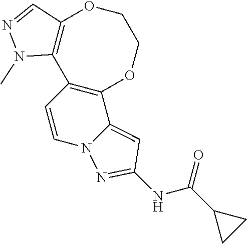 [SMILES: Cn1ncc2c1-c1ccn3nc(NC(=O)C4CC4)cc3c1OCCO2] | LCMS-1 | 340.2 | 0.447 |
| I-4 | 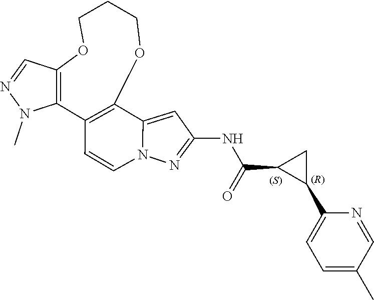 | LCMS-1 | 445.1 | 0.425 |
| I-5 | 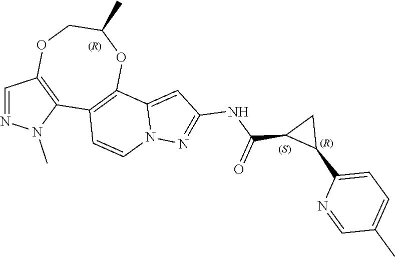 | LCMS-1 | 445.2 | 0.374 |
| I-6 | 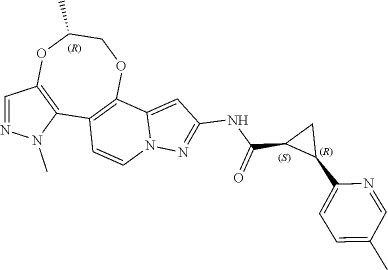 | LCMS-1 | 445.2 | 0.383 |
| I-7 | 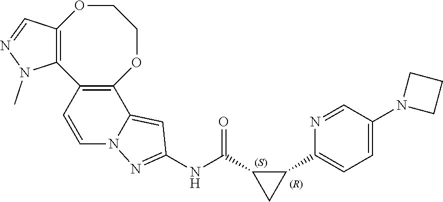 | LCMS-1 | 472.0 | 0.447 |
| I-8 | 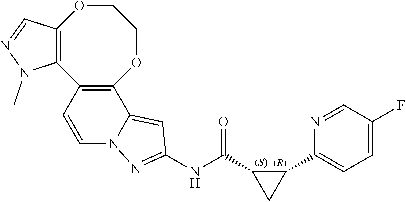 | LCMS-1 | 435.1 | 0.429 |
| I-9 | 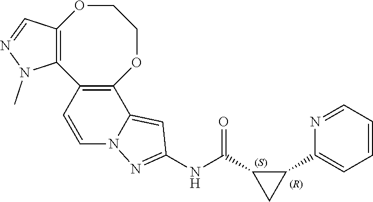 | LCMS-1 | 417.1 | 0.39 |
| I-10 | 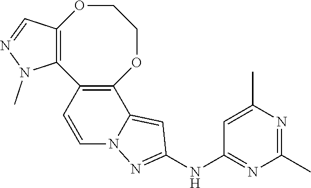 [SMILES: Cc1cc(Nc2cc3c4c(ccn3n2)-c2c(cnn2C)OCCO4)nc(C)n1] | LCMS-1 | 378.0 | 0.414 |
| I-11 | 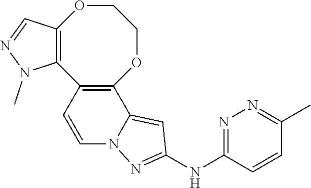 [SMILES: Cc1ccc(Nc2cc3c4c(ccn3n2)-c2c(cnn2C)OCCO4)nn1] | LCMS-1 | 364.1 | 0.399 |
| I-12 | 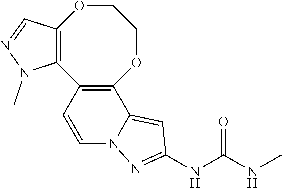 [SMILES: CNC(=O)Nc1cc2c3c(ccn2n1)-c1c(cnn1C)OCCO3] | LCMS-1 | 329.1 | 0.404 |
| I-13 | 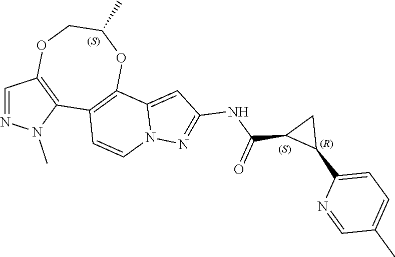 | LCMS-1 | 445.2 | 0.411 |
| I-14 | 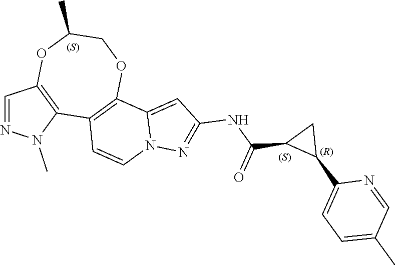 | LCMS-1 | 445.2 | 0.41 |
| I-15 | 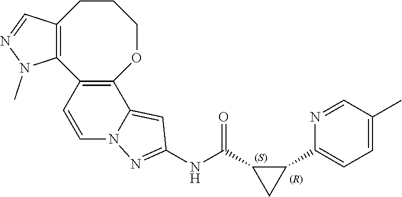 | LCMS-1 | 429.0 | 0.438 |
| I-16 | 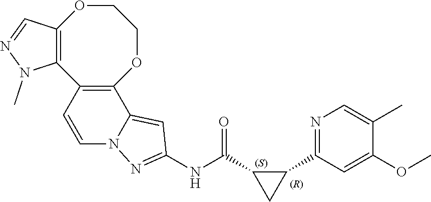 | LCMS-2 | 461.2 | 0.529 |
| I-17 | 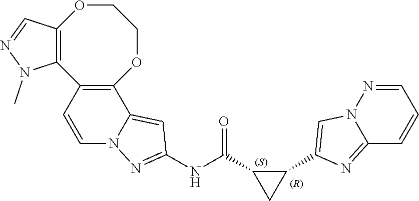 | LCMS-1 | 457.2 | 0.407 |
| I-18 | 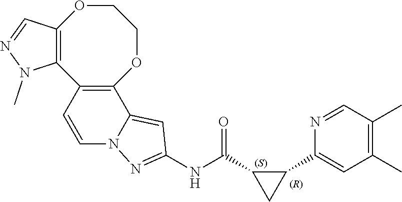 | LCMS-1 | 445.1 | 0.437 |
| I-19 | 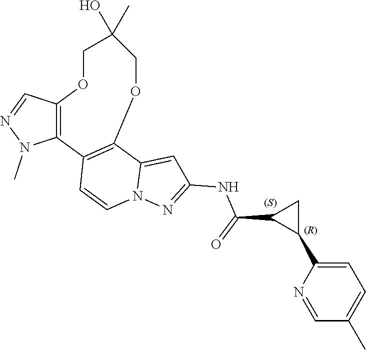 | LCMS-1 | 475.2 | 0.407 |
| I-20 | 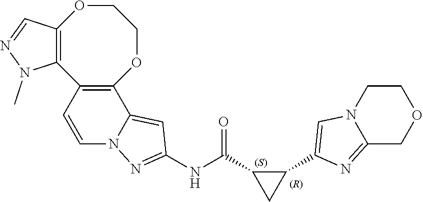 | LCMS-1 | 462.1 | 0.42 |
| I-21 | 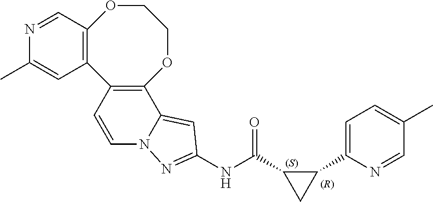 | LCMS-1 | 442.1 | 0.39 |
| I-22 | 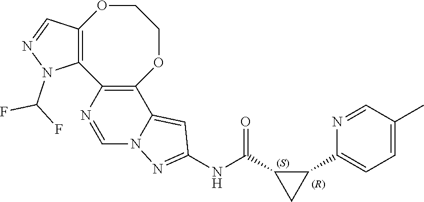 | LCMS-1 | 468 | 0.47 |
| I-23 | 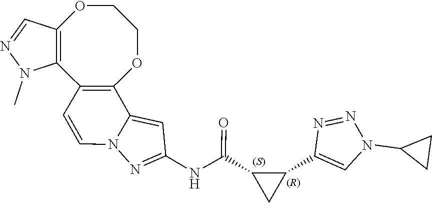 | LCMS-1 | 447.1 | 0.448 |
| I-24 | 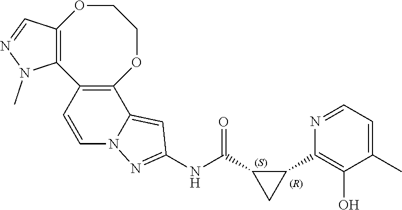 | LCMS-1 | 447.2 | 0.37 |
| I-25 | 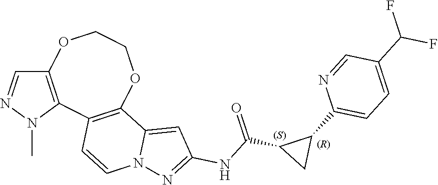 | LCMS-1 | 467.1 | 0.441 |
| I-26 | 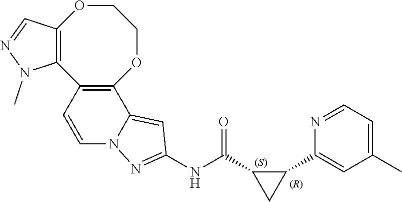 | LCMS-1 | 4321.2 | 0.401 |
| I-27 | 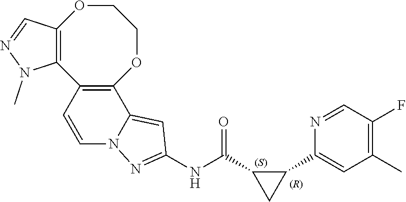 | LCMS-1 | 449.2 | 0.432 |
| I-28 | 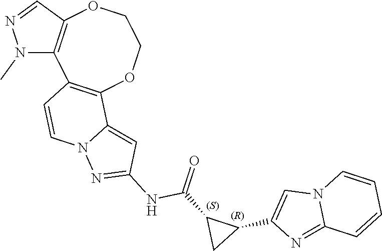 | LCMS-1 | 456.1 | 0.406 |
| I-29 | 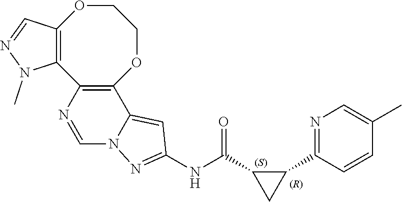 | LCMS-1 | 432 | 0.429 |
| I-30 | 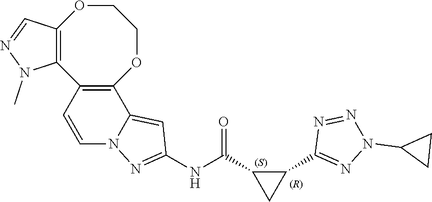 | LCMS-1 | 448.2 | 0.441 |
| I-31 | 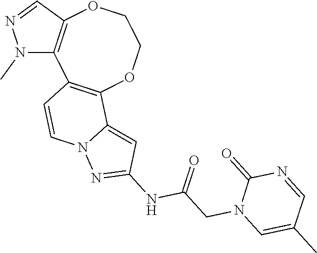 | LCMS-1 | 422.1 | 0.381 |
| I-32 | 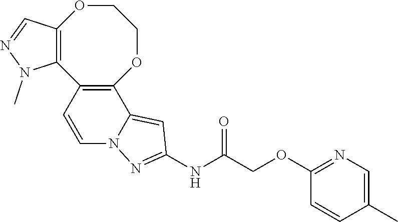 [SMILES: Cc1ccc(OCC(=O)Nc2cc3c4c(ccn3n2)-c2c(cnn2C)OCCO4)nc1] | LCMS-1 | 421.1 | 0.475 |
| I-33 | 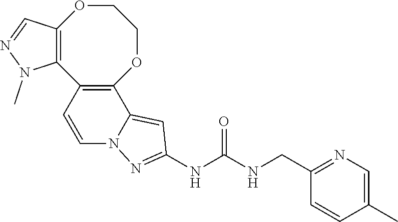 [SMILES: Cc1ccc(CNC(=O)Nc2cc3c4c(ccn3n2)-c2c(cnn2C)OCCO4)nc1] | LCMS-1 | 420.1 | 0.37 |
| I-34 | 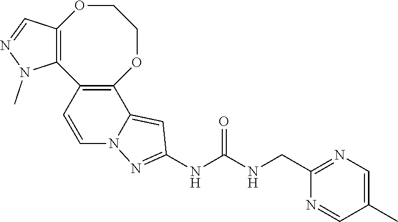 [SMILES: Cc1cnc(CNC(=O)Nc2cc3c4c(ccn3n2)-c2c(cnn2C)OCCO4)nc1] | LCMS-1 | 421.2 | 0.414 |
| I-35 | 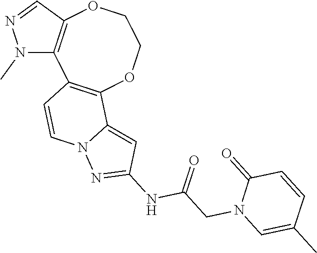 | LCMS-1 | 421.1 | 0.42 |
| I-36 | 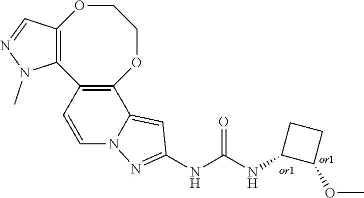 [SMILES: CO[C@H]1CC[C@H]1NC(=O)Nc1cc2c3c(ccn2n1)-c1c(cnn1C)OCCO3] | LCMS-2 | 399.2 | 0.479 |
| I-37 | 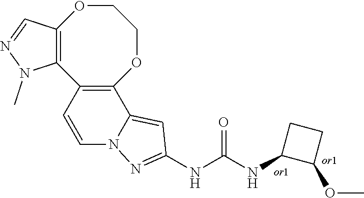 [SMILES: CO[C@@H]1CC[C@@H]1NC(=O)Nc1cc2c3c(ccn2n1)-c1c(cnn1C)OCCO3] | LCMS-2 | 399.3 | 0.484 |
| I-38 | 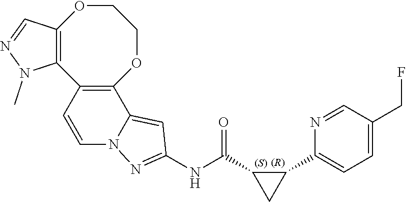 | LCMS-1 | 449.2 | 0.389 |
| I-39 | 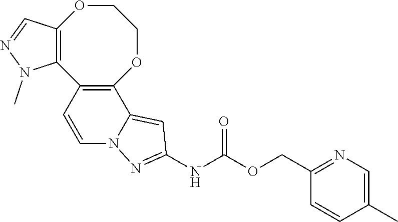 [SMILES: Cc1ccc(COC(=O)Nc2cc3c4c(ccn3n2)-c2c(cnn2C)OCCO4)nc1] | LCMS-1 | 421 | 0.404 |
| I-40 | 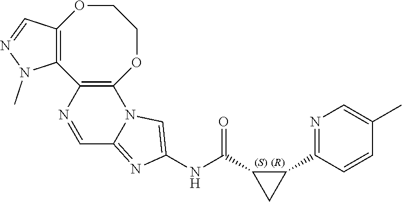 | LCMS-1 | 432 | 0.411 |
| I-41 | 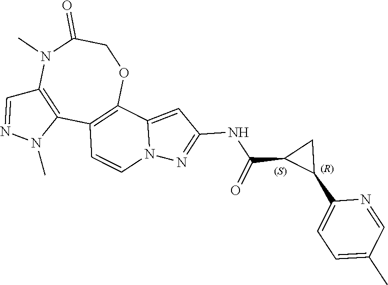 | LCMS-1 | 458.3 | 0.38 |
| I-42 | 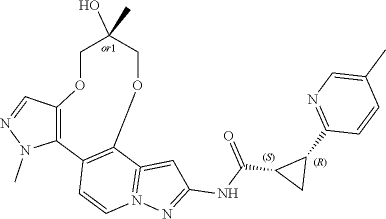 | LCMS-1 | 475.3 | 0.371 |
| I-43 | 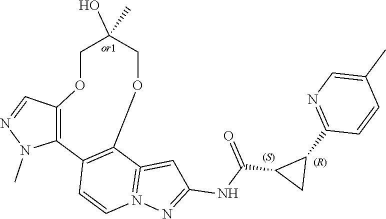 | LCMS-1 | 475.3 | 0.378 |
| I-44 | 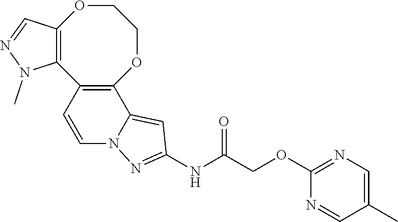 [SMILES: Cc1cnc(OCC(=O)Nc2cc3c4c(ccn3n2)-c2c(cnn2C)OCCO4)nc1] | LCMS-1 | 422.1 | 0.437 |
| I-45 | 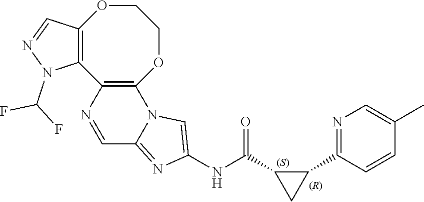 | LCMS-1 | 468.2 | 0.412 |
| I-46 | 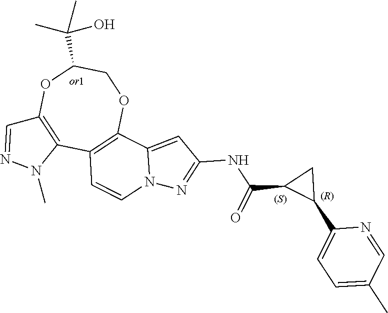 | LCMS-1 | 489.3 | 0.395 |
| I-47 | 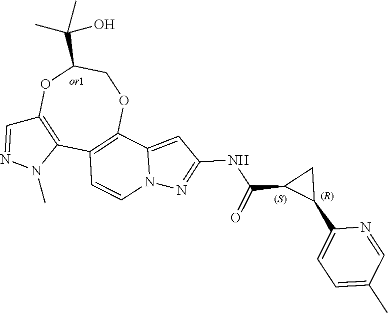 | LCMS-1 | 489.3 | 0.391 |
| I-48 | 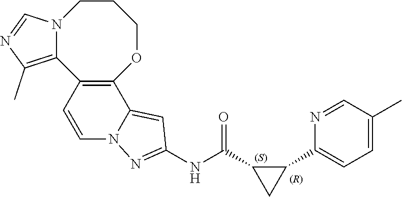 | LCMS 1 | 429.3 | 0.343 |
| I-49 | 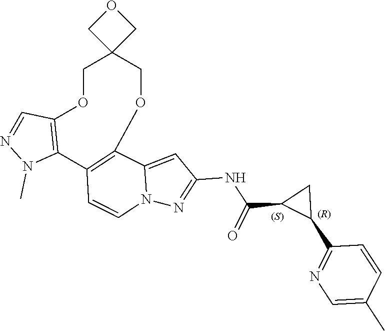 | LCMS 1 | 487.2 | 0.37 |
| I-50 | 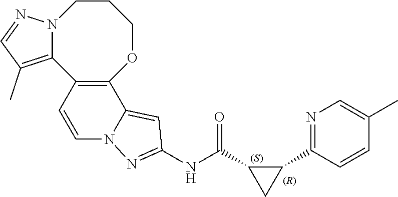 | LCMS 1 | 429.3 | 0.405 |
| I-51 | 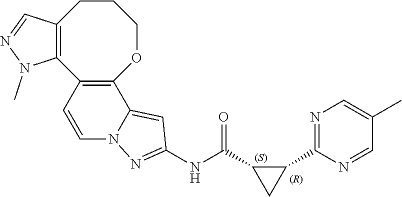 | LCMS 1 | 430.2 | 0.422 |
| I-52 | 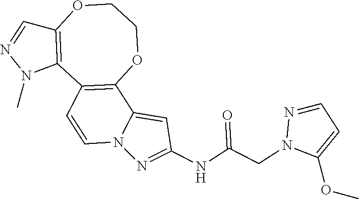 [SMILES: COc1ccnn1CC(=O)Nc1cc2c3c(ccn2n1)-c1c(cnn1C)OCCO3] | LCMS 1 | 410.1 | 0.421 |
| I-53 | 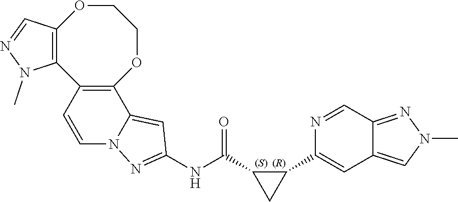 | LCMS 1 | 471.2 | 0.375 |
| I-54 | 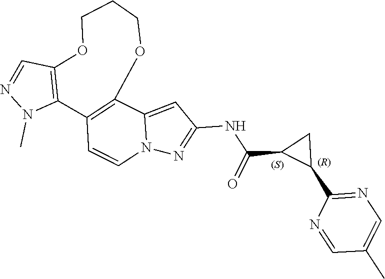 | LCMS 1 | 446.2 | 0.411 |
| I-55 | 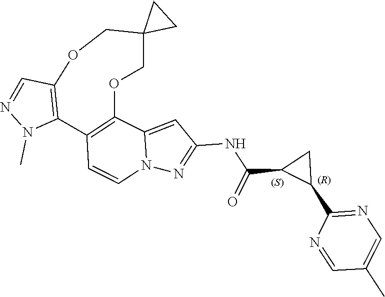 | LCMS 1 | 472.2 | 0.426 |
| I-56 | 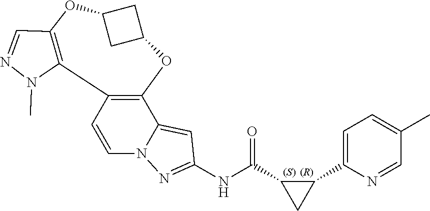 | LCMS 1 | 457.4 | 0.4 |
| I-57 | 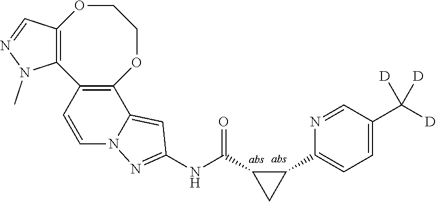 [SMILES: [2H]C([2H])([2H])c1ccc([C@@H]2C[C@@H]2C(=O)Nc2cc3c4c(ccn3n2)-c2c(cnn2C)OCCO4)nc1] | LCMS-1 | 434.2 | 0.379 |
| I-58 | 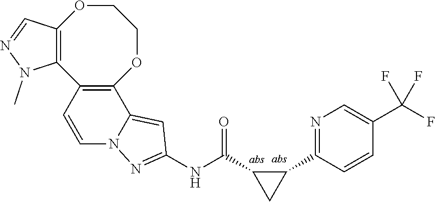 [SMILES: Cn1ncc2c1-c1ccn3nc(NC(=O)[C@H]4C[C@H]4c4ccc(C(F)(F)F)cn4)cc3c1OCCO2] | LCMS-1 | 485.1 | 0.483 |
| I-59 | 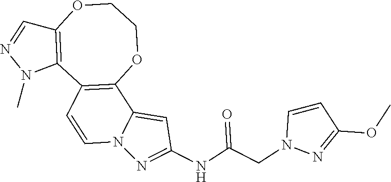 [SMILES: COc1ccn(CC(=O)Nc2cc3c4c(ccn3n2)-c2c(cnn2C)OCCO4)n1] | LCMS-1 | 410.1 | 0.425 |
| I-60 | 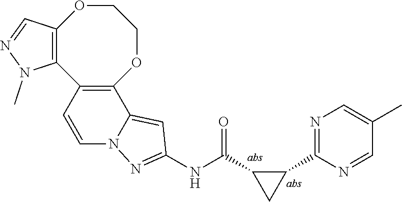 [SMILES: Cc1cnc([C@@H]2C[C@@H]2C(=O)Nc2cc3c4c(ccn3n2)-c2c(cnn2C)OCCO4)nc1] | LCMS-1 | 432.2 | 0.407 |
| I-61 | 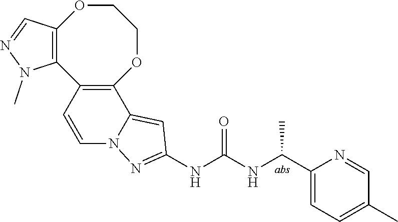 [SMILES: Cc1ccc([C@@H](C)NC(=O)Nc2cc3c4c(ccn3n2)-c2c(cnn2C)OCCO4)nc1] | LCMS-1 | 434.1 | 0.376 |
| I-62 | 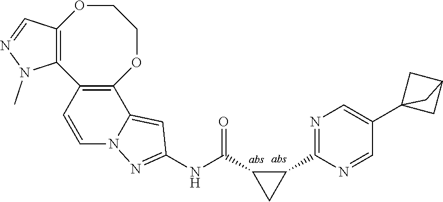 [SMILES: Cn1ncc2c1-c1ccn3nc(NC(=O)[C@H]4C[C@H]4c4ncc(C56CC(C5)C6)cn4)cc3c1OCCO2] | LCMS-1 | 484.3 | 0.474 |
| I-63 | 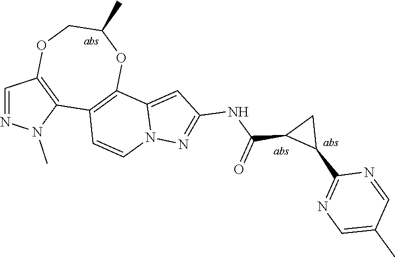 [SMILES: Cc1cnc([C@@H]2C[C@@H]2C(=O)Nc2cc3c4c(ccn3n2)-c2c(cnn2C)OC[C@@H](C)O4)nc1] | LCMS-1 | 446.3 | 0.421 |
| I-64 | 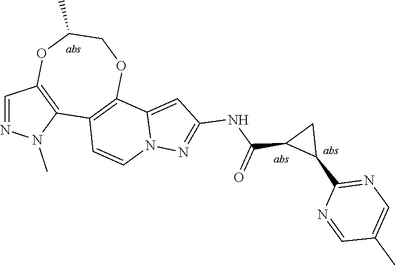 [SMILES: Cc1cnc([C@@H]2C[C@@H]2C(=O)Nc2cc3c4c(ccn3n2)-c2c(cnn2C)O[C@H](C)CO4)nc1] | LCMS-1 | 446.3 | 0.43 |
| I-65 | 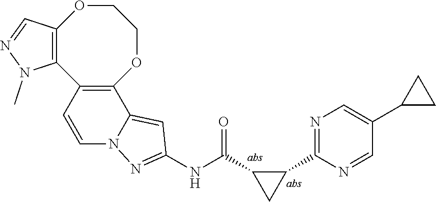 [SMILES: Cn1ncc2c1-c1ccn3nc(NC(=O)[C@H]4C[C@H]4c4ncc(C5CC5)cn4)cc3c1OCCO2] | LCMS-1 | 458.2 | 0.438 |
| I-66 | 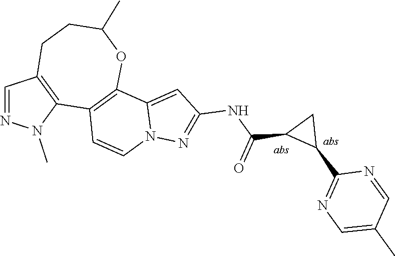 [SMILES: Cc1cnc([C@@H]2C[C@@H]2C(=O)Nc2cc3c4c(ccn3n2)-c2c(cnn2C)CCC(C)O4)nc1] | LCMS-1 | 444.1 | 0.479 |
| I-67 | 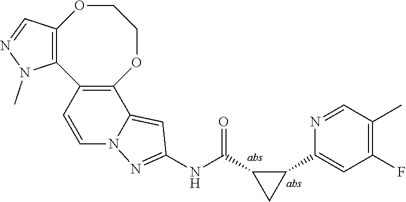 [SMILES: Cc1cnc([C@@H]2C[C@@H]2C(=O)Nc2cc3c4c(ccn3n2)-c2c(cnn2C)OCCO4)cc1F] | LCMS-1 | 449.1 | 0.403 |
| I-68 | 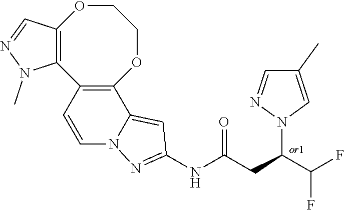 [SMILES: Cc1cnn([C@H](CC(=O)Nc2cc3c4c(ccn3n2)-c2c(cnn2C)OCCO4)C(F)F)c1] | LCMS-2 | 458.2 | 0.561 |
| I-69 | 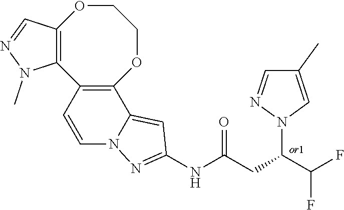 [SMILES: Cc1cnn([C@@H](CC(=O)Nc2cc3c4c(ccn3n2)-c2c(cnn2C)OCCO4)C(F)F)c1] | LCMS-2 | 458.3 | 0.56 |
| I-70 | 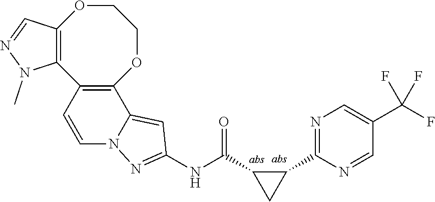 [SMILES: Cn1ncc2c1-c1ccn3nc(NC(=O)[C@H]4C[C@H]4c4ncc(C(F)(F)F)cn4)cc3c1OCCO2] | LCMS-2 | 484.2 | 0.511 |
| I-71 | 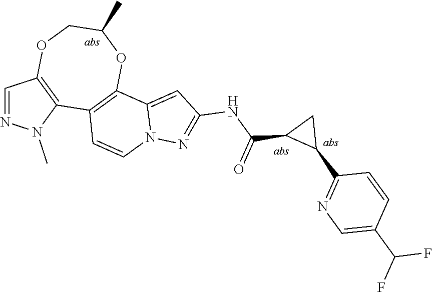 [SMILES: C[C@@H]1COc2cnn(C)c2-c2ccn3nc(NC(=O)[C@H]4C[C@H]4c4ccc(C(F)F)cn4)cc3c2O1] | LCMS-1 | 481.2 | 0.451 |
| I-72 | 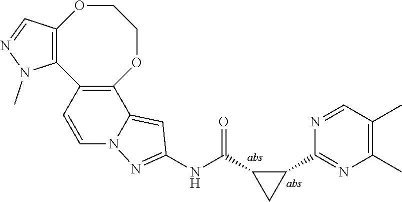 [SMILES: Cc1cnc([C@@H]2C[C@@H]2C(=O)Nc2cc3c4c(ccn3n2)-c2c(cnn2C)OCCO4)nc1C] | LCMS-1 | 446.2 | 0.401 |
| I-73 | 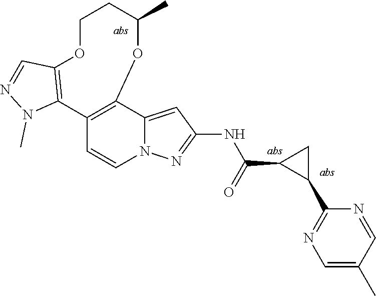 [SMILES: Cc1cnc([C@@H]2C[C@@H]2C(=O)Nc2cc3c4c(ccn3n2)-c2c(cnn2C)OCC[C@@H](C)O4)nc1] | LCMS-1 | 460.1 | 0.429 |
| I-74 | 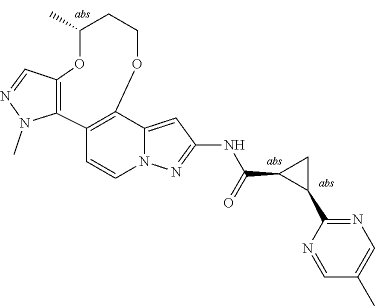 [SMILES: Cc1cnc([C@@H]2C[C@@H]2C(=O)Nc2cc3c4c(ccn3n2)-c2c(cnn2C)O[C@H](C)CCO4)nc1] | LCMS-1 | 460.2 | 0.436 |
| I-75 | 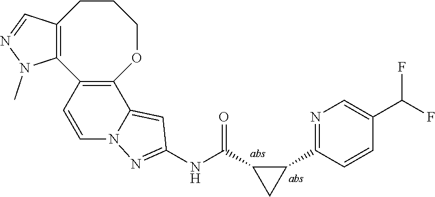 [SMILES: Cn1ncc2c1-c1ccn3nc(NC(=O)[C@H]4C[C@H]4c4ccc(C(F)F)cn4)cc3c1OCCC2] | LCMS-1 | 465.3 | 0.456 |
| I-76 | 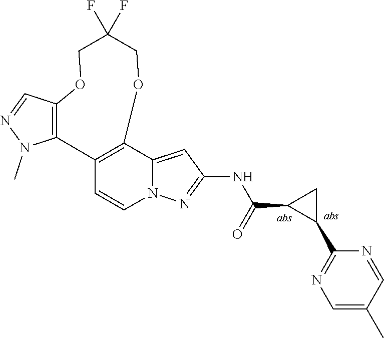 [SMILES: Cc1cnc([C@@H]2C[C@@H]2C(=O)Nc2cc3c4c(ccn3n2)-c2c(cnn2C)OCC(F)(F)CO4)nc1] | LCMS-1 | 482.3 | 0.43 |
| I-77 | 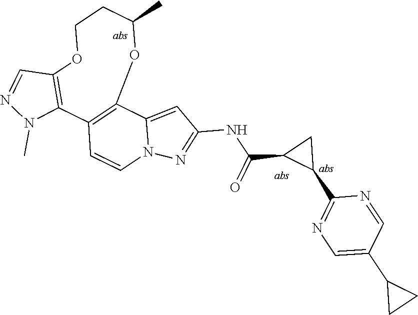 [SMILES: C[C@@H]1CCOc2cnn(C)c2-c2ccn3nc(NC(=O)[C@H]4C[C@H]4c4ncc(C5CC5)cn4)cc3c2O1] | LCMS-1 | 486.1 | 0.456 |
| I-78 | 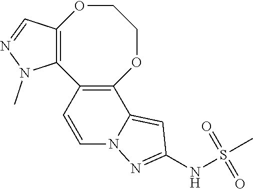 [SMILES: Cn1ncc2c1-c1ccn3nc(NS(C)(=O)=O)cc3c1OCCO2] | LCMS-1 | 349.9 | 0.406 |
| I-79 | 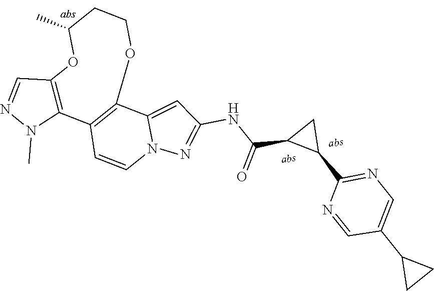 [SMILES: C[C@@H]1CCOc2c(ccn3nc(NC(=O)[C@H]4C[C@H]4c4ncc(C5CC5)cn4)cc23)-c2c(cnn2C)O1] | LCMS-1 | 486.2 | 0.465 |
| I-80 | 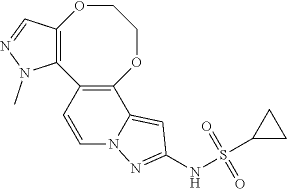 [SMILES: Cn1ncc2c1-c1ccn3nc(NS(=O)(=O)C4CC4)cc3c1OCCO2] | LCMS-1 | 376 | 0.438 |
| I-81 | 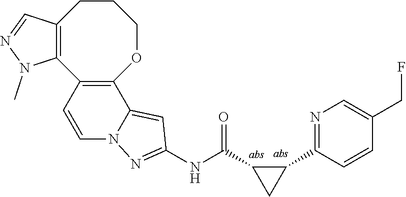 [SMILES: Cn1ncc2c1-c1ccn3nc(NC(=O)[C@H]4C[C@H]4c4ccc(CF)cn4)cc3c1OCCC2] | LCMS-1 | 447.3 | 0.417 |
| I-82 | 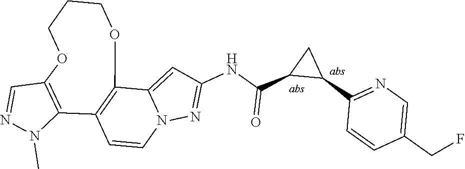 [SMILES: Cn1ncc2c1-c1ccn3nc(NC(=O)[C@H]4C[C@H]4c4ccc(CF)cn4)cc3c1OCCCO2] | LCMS-1 | 463.2 | 0.39 |
| I-83 | 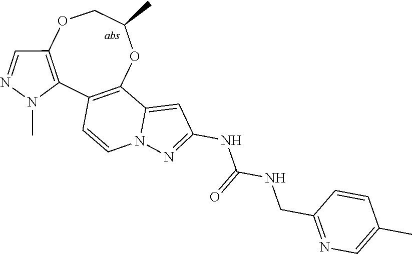 [SMILES: Cc1ccc(CNC(=O)Nc2cc3c4c(ccn3n2)-c2c(cnn2C)OC[C@@H](C)O4)nc1] | LCMS-2 | 434.3 | 0.528 |
| I-84 | 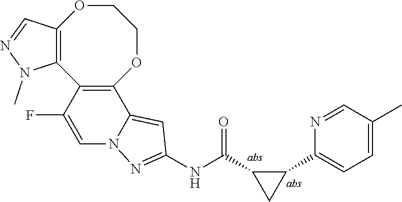 [SMILES: Cc1ccc([C@@H]2C[C@@H]2C(=O)Nc2cc3c4c(c(F)cn3n2)-c2c(cnn2C)OCCO4)nc1] | LCMS-1 | 449.1 | 0.437 |
| I-85 | 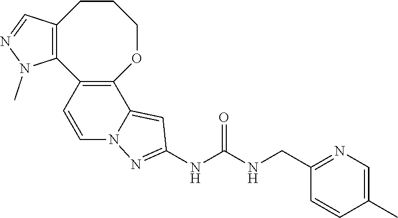 [SMILES: Cc1ccc(CNC(=O)Nc2cc3c4c(ccn3n2)-c2c(cnn2C)CCCO4)nc1] | LCMS-1 | 418.3 | 0.392 |
| I-86 | 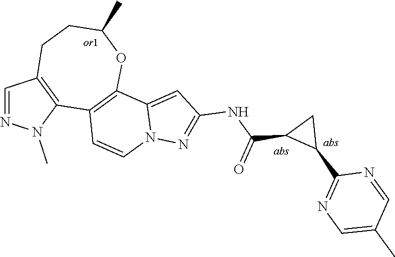 [SMILES: Cc1cnc([C@@H]2C[C@@H]2C(=O)Nc2cc3c4c(ccn3n2)-c2c(cnn2C)CC[C@@H](C)O4)nc1] | LCMS-1 | 444.1 | 0.477 |
| I-87 | 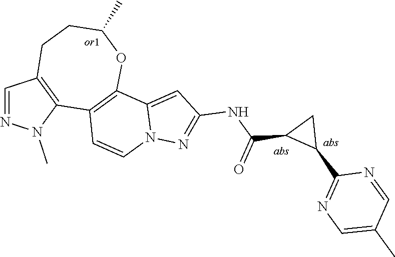 [SMILES: Cc1cnc([C@@H]2C[C@@H]2C(=O)Nc2cc3c4c(ccn3n2)-c2c(cnn2C)CC[C@H](C)O4)nc1] | LCMS-1 | 444.1 | 0.485 |
| I-88 | 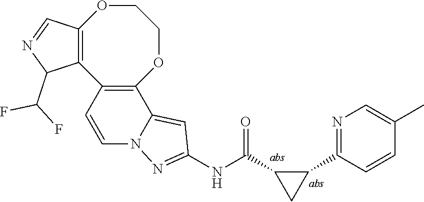 [SMILES: Cc1ccc([C@@H]2C[C@@H]2C(=O)Nc2cc3c4c(ccn3n2)C2=C(C=NC2C(F)F)OCCO4)nc1] | LCMS-1 | 467.2 | 0.421 |
| I-89 | 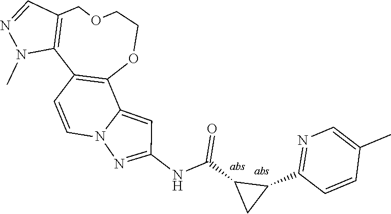 [SMILES: Cc1ccc([C@@H]2C[C@@H]2C(=O)Nc2cc3c4c(ccn3n2)-c2c(cnn2C)COCCO4)nc1] | LCMS-1 | 445.2 | 0.366 |
| I-90 | 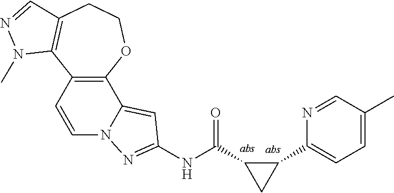 | LCMS-1 | 415.1 | 0.39 |
| I-91 | 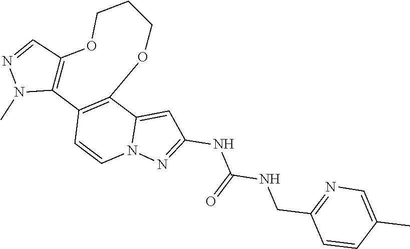 | LCMS-1 | 434.2 | 0.382 |
| I-92 | 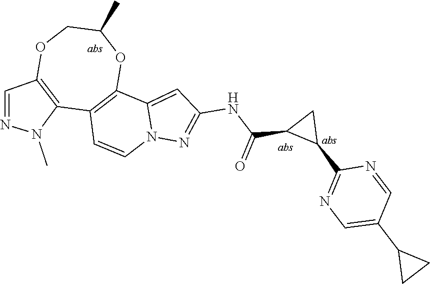 [SMILES: C[C@@H]1COc2cnn(C)c2-c2ccn3nc(NC(=O)[C@H]4C[C@H]4c4ncc(C5CC5)cn4)cc3c2O1] | LCMS-1 | 472.1 | 0.461 |
| I-93 | 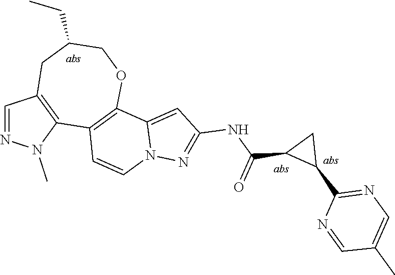 [SMILES: CC[C@@H]1COc2c(ccn3nc(NC(=O)[C@H]4C[C@H]4c4ncc(C)cn4)cc23)-c2c(cnn2C)C1] | LCMS-1 | 460.2 | 0.447 |
| I-94 | 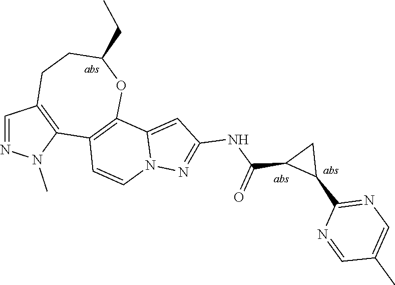 [SMILES: CC[C@@H]1CCc2cnn(C)c2-c2ccn3nc(NC(=O)[C@H]4C[C@H]4c4ncc(C)cn4)cc3c2O1] | LCMS-1 | 460.2 | 0.451 |
| I-95 | 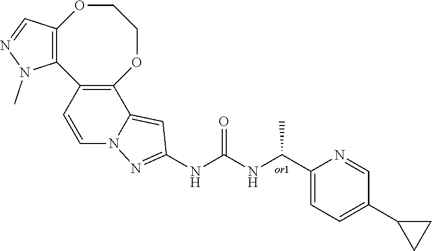 [SMILES: C[C@@H](NC(=O)Nc1cc2c3c(ccn2n1)-c1c(cnn1C)OCCO3)c1ccc(C2CC2)cn1] | LCMS-1 | 460.3 | 0.41 |
| I-96 | 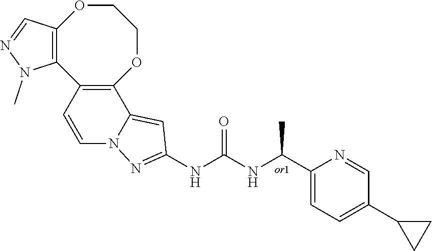 [SMILES: C[C@H](NC(=O)Nc1cc2c3c(ccn2n1)-c1c(cnn1C)OCCO3)c1ccc(C2CC2)cn1] | LCMS-1 | 460.4 | 0.419 |
| I-97 | 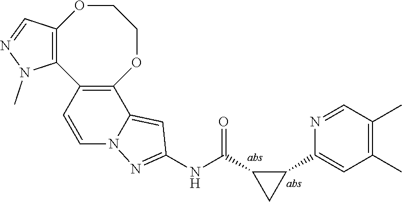 [SMILES: Cc1cnc([C@@H]2C[C@@H]2C(=O)Nc2cc3c4c(ccn3n2)-c2c(cnn2C)OCCO4)cc1C] | LCMS-1 | 444.1 | 0.423 |
| I-98 | 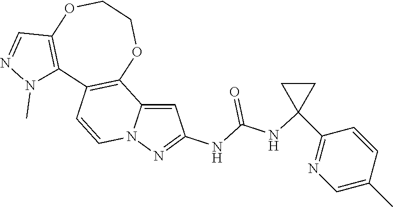 [SMILES: Cc1ccc(C2(NC(=O)Nc3cc4c5c(ccn4n3)-c3c(cnn3C)OCCO5)CC2)nc1] | LCMS-1 | 446.2 | 0.387 |
| I-99 | 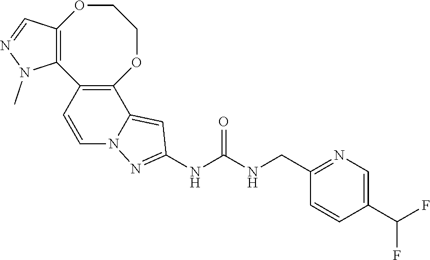 [SMILES: Cn1ncc2c1-c1ccn3nc(NC(=O)NCc4ccc(C(F)F)cn4)cc3c1OCCO2] | LCMS-2 | 456.2 | 0.523 |
| I-100 | 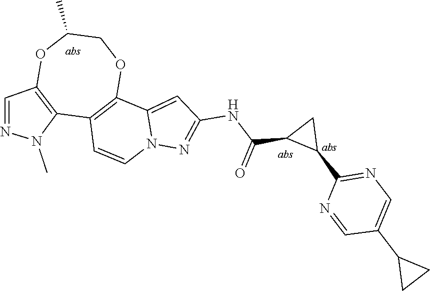 [SMILES: C[C@@H]1COc2c(ccn3nc(NC(=O)[C@H]4C[C@H]4c4ncc(C5CC5)cn4)cc23)-c2c(cnn2C)O1] | LCMS-1 | 472.2 | 0.457 |
| I-101 | 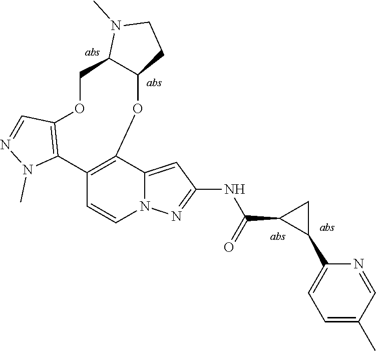 [SMILES: Cc1ccc([C@@H]2C[C@@H]2C(=O)Nc2cc3c4c(ccn3n2)-c2c(cnn2C)OC[C@@H]2[C@@H](CCN2C)O4)nc1] | LCMS-1 | 500.2 | 0.335 |
| I-102 | 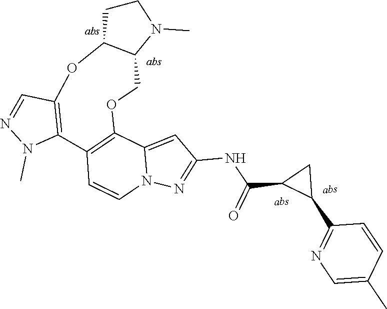 [SMILES: Cc1ccc([C@@H]2C[C@@H]2C(=O)Nc2cc3c4c(ccn3n2)-c2c(cnn2C)O[C@@H]2CCN(C)[C@@H]2CO4)nc1] | LCMS-1 | 500.3 | 0.33 |
| I-103 | 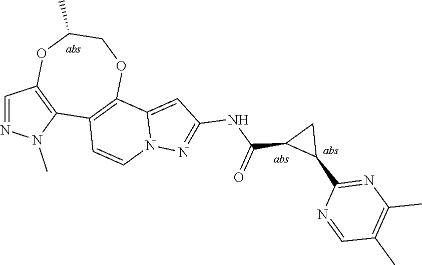 [SMILES: Cc1cnc([C@@H]2C[C@@H]2C(=O)Nc2cc3c4c(ccn3n2)-c2c(cnn2C)O[C@H](C)CO4)nc1C] | LCMS-1 | 460.2 | 0.425 |
| I-104 | 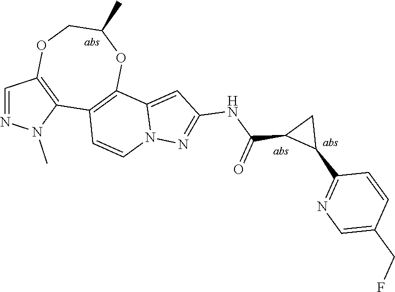 [SMILES: C[C@@H]1COc2cnn(C)c2-c2ccn3nc(NC(=O)[C@H]4C[C@H]4c4ccc(CF)cn4)cc3c2O1] | LCMS-2 | 463.3 | 0.543 |
| I-105 | 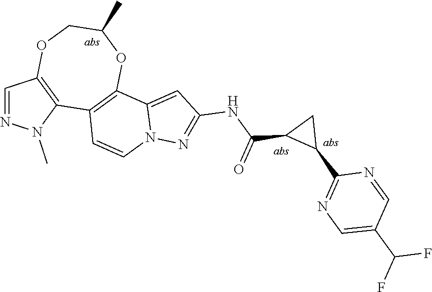 [SMILES: C[C@@H]1COc2cnn(C)c2-c2ccn3nc(NC(=O)[C@H]4C[C@H]4c4ncc(C(F)F)cn4)cc3c2O1] | LCMS-2 | 482.3 | 0.517 |
| I-106 | 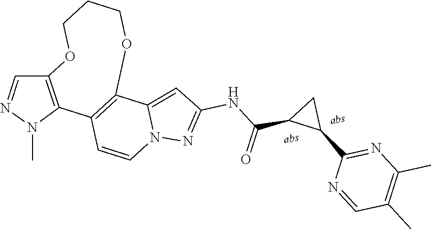 [SMILES: Cc1cnc([C@@H]2C[C@@H]2C(=O)Nc2cc3c4c(ccn3n2)-c2c(cnn2C)OCCCO4)nc1C] | LCMS-2 | 460.3 | 0.482 |
| I-107 | 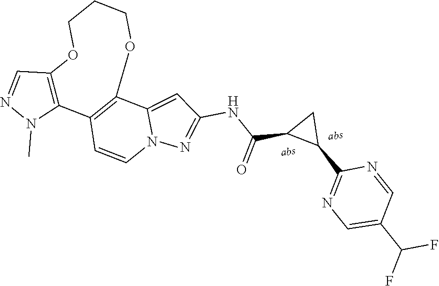 [SMILES: Cn1ncc2c1-c1ccn3nc(NC(=O)[C@H]4C[C@H]4c4ncc(C(F)F)cn4)cc3c1OCCCO2] | LCMS-1 | 482.2 | 0.446 |
| I-108 | 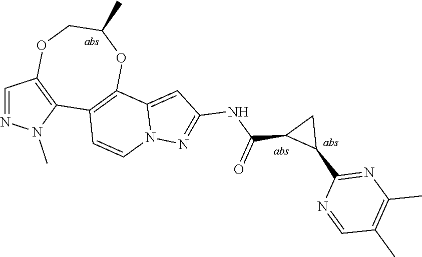 [SMILES: Cc1cnc([C@@H]2C[C@@H]2C(=O)Nc2cc3c4c(ccn3n2)-c2c(cnn2C)OC[C@@H](C)O4)nc1C] | LCMS-1 | 460.2 | 0.419 |
| I-109 | 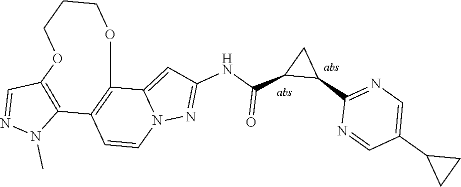 [SMILES: Cn1ncc2c1-c1ccn3nc(NC(=O)[C@H]4C[C@H]4c4ncc(C5CC5)cn4)cc3c1OCCCO2] | LCMS-1 | 472.3 | 0.448 |
| I-110 | 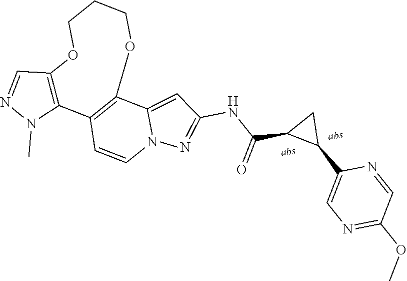 [SMILES: COc1cnc([C@@H]2C[C@@H]2C(=O)Nc2cc3c4c(ccn3n2)-c2c(cnn2C)OCCCO4)cn1] | LCMS-1 | 462.1 | 0.488 |
| I-111 | 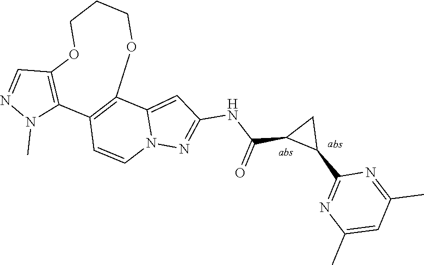 [SMILES: Cc1cc(C)nc([C@@H]2C[C@@H]2C(=O)Nc2cc3c4c(ccn3n2)-c2c(cnn2C)OCCCO4)n1] | LCMS-1 | 460.2 | 0.406 |
| I-112 | 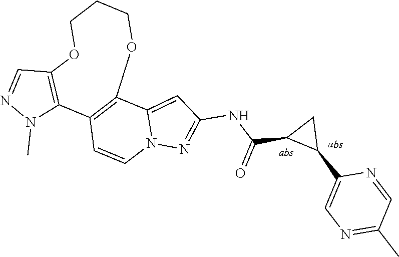 [SMILES: Cc1cnc([C@@H]2C[C@@H]2C(=O)Nc2cc3c4c(ccn3n2)-c2c(cnn2C)OCCCO4)cn1] | LCMS-1 | 446.2 | 0.428 |
| I-113 | 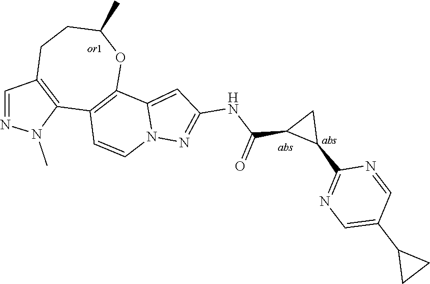 [SMILES: C[C@@H]1CCc2cnn(C)c2-c2ccn3nc(NC(=O)[C@H]4C[C@H]4c4ncc(C5CC5)cn4)cc3c2O1] | LCMS-1 | 470.2 | 0.475 |
| I-114 | 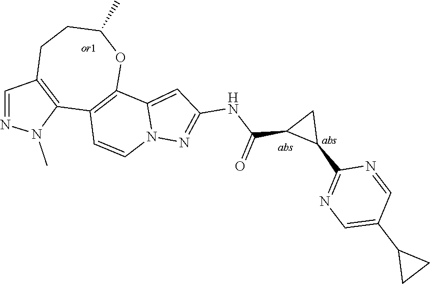 [SMILES: C[C@H]1CCc2cnn(C)c2-c2ccn3nc(NC(=O)[C@H]4C[C@H]4c4ncc(C5CC5)cn4)cc3c2O1] | LCMS-1 | 470.2 | 0.479 |
| I-115 | 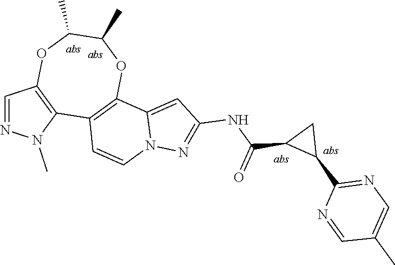 [SMILES: Cc1cnc([C@@H]2C[C@@H]2C(=O)Nc2cc3c4c(ccn3n2)-c2c(cnn2C)O[C@H](C)[C@@H](C)O4)nc1] | LCMS-1 | 460.2 | 0.437 |
| I-116 | 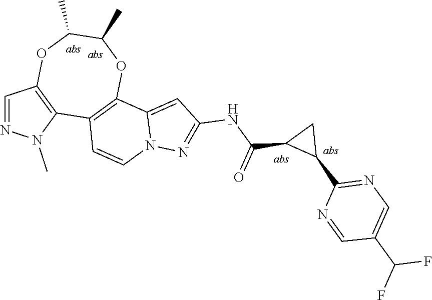 [SMILES: C[C@H]1Oc2cnn(C)c2-c2ccn3nc(NC(=O)[C@H]4C[C@H]4c4ncc(C(F)F)cn4)cc3c2O[C@@H]1C] | LCMS-1 | 496.3 | 0.471 |
| I-117 | 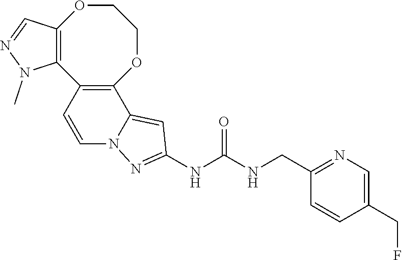 [SMILES: Cn1ncc2c1-c1ccn3nc(NC(=O)NCc4ccc(CF)cn4)cc3c1OCCO2] | LCMS-2 | 438.2 | 0.477 |
| I-118 | 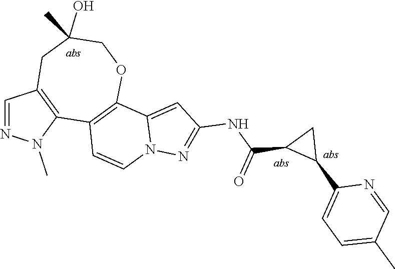 [SMILES: Cc1ccc([C@@H]2C[C@@H]2C(=O)Nc2cc3c4c(ccn3n2)-c2c(cnn2C)C[C@](C)(O)CO4)nc1] | LCMS-1 | 459.2 | 0.367 |
| I-119 | 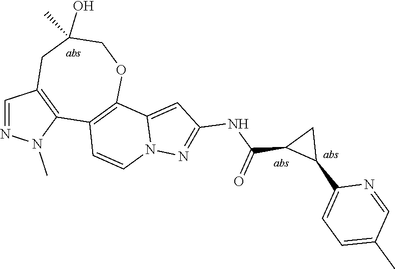 [SMILES: Cc1ccc([C@@H]2C[C@@H]2C(=O)Nc2cc3c4c(ccn3n2)-c2c(cnn2C)C[C@@](C)(O)CO4)nc1] | LCMS-1 | 459.3 | 0.37 |
| I-120 | 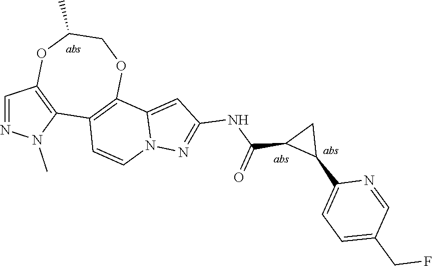 [SMILES: C[C@@H]1COc2c(ccn3nc(NC(=O)[C@H]4C[C@H]4c4ccc(CF)cn4)cc23)-c2c(cnn2C)O1] | LCMS-1 | 463.2 | 0.405 |
| I-121 | 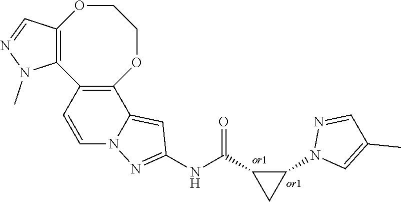 [SMILES: Cc1cnn([C@@H]2C[C@@H]2C(=O)Nc2cc3c4c(ccn3n2)-c2c(cnn2C)OCCO4)c1] | LCMS-2 | 420.3 | 0.51 |
| I-122 | 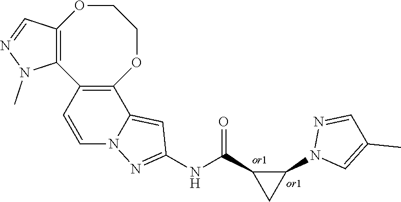 [SMILES: Cc1cnn([C@H]2C[C@H]2C(=O)Nc2cc3c4c(ccn3n2)-c2c(cnn2C)OCCO4)c1] | LCMS-2 | 420.3 | 0.513 |
| I-123 | 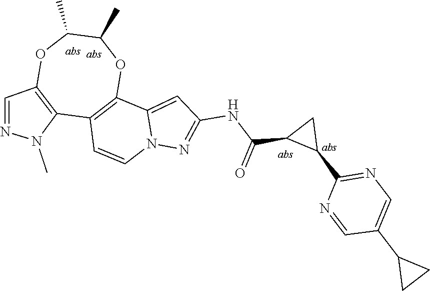 [SMILES: C[C@H]1Oc2cnn(C)c2-c2ccn3nc(NC(=O)[C@H]4C[C@H]4c4ncc(C5CC5)cn4)cc3c2O[C@@H]1C] | LCMS-1 | 486.2 | 0.461 |
| I-124 | 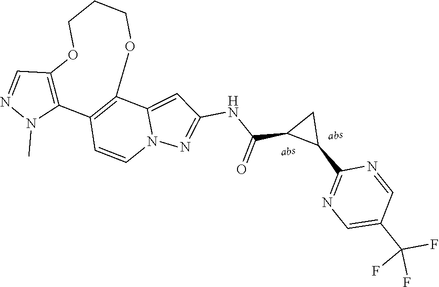 [SMILES: Cn1ncc2c1-c1ccn3nc(NC(=O)[C@H]4C[C@H]4c4ncc(C(F)(F)F)cn4)cc3c1OCCCO2] | LCMS-1 | 500.2 | 0.469 |
| I-125 | 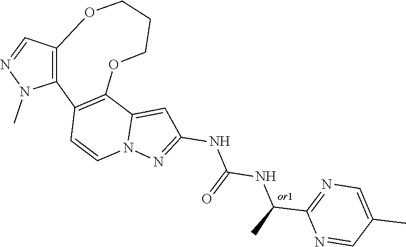 [SMILES: Cc1cnc([C@@H](C)NC(=O)Nc2cc3c4c(ccn3n2)-c2c(cnn2C)OCCCO4)nc1] | LCMS-1 | 449.1 | 0.438 |
| I-126 | 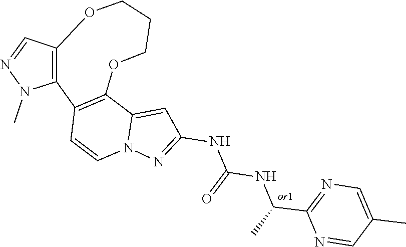 [SMILES: Cc1cnc([C@H](C)NC(=O)Nc2cc3c4c(ccn3n2)-c2c(cnn2C)OCCCO4)nc1] | LCMS-1 | 449.1 | 0.436 |
| I-127 | 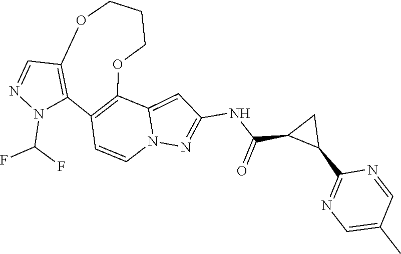 [SMILES: Cc1cnc([C@@H]2C[C@@H]2C(=O)Nc2cc3c4c(ccn3n2)-c2c(cnn2C(F)F)OCCCO4)nc1] | LCMS-1 | 482.1 | 0.46 |
| I-128 | 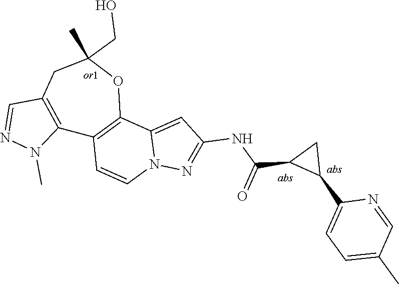 [SMILES: Cc1ccc([C@@H]2C[C@@H]2C(=O)Nc2cc3c4c(ccn3n2)-c2c(cnn2C)C[C@](C)(CO)O4)nc1] | LCMS-1 | 459.2 | 0.369 |
| I-129 | 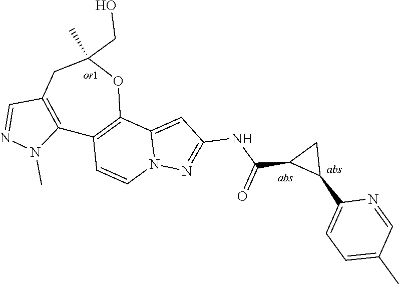 [SMILES: Cc1ccc([C@@H]2C[C@@H]2C(=O)Nc2cc3c4c(ccn3n2)-c2c(cnn2C)C[C@@](C)(CO)O4)nc1] | LCMS-1 | 459.2 | 0.369 |
| I-130 | 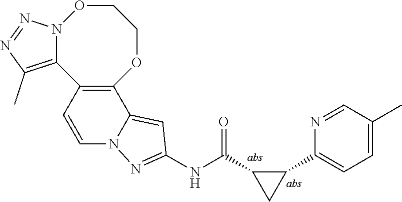 [SMILES: Cc1ccc([C@@H]2C[C@@H]2C(=O)Nc2cc3c4c(ccn3n2)-c2c(C)nnn2OCCO4)nc1] | LCMS-1 | 430.2 | 0.376 |
| I-131 | 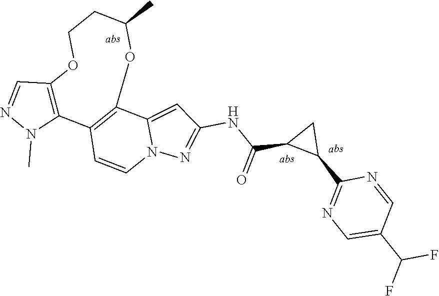 [SMILES: C[C@@H]1CCOc2cnn(C)c2-c2ccn3nc(NC(=O)[C@H]4C[C@H]4c4ncc(C(F)F)cn4)cc3c2O1] | LCMS-1 | 496.2 | 0.466 |
| I-132 | 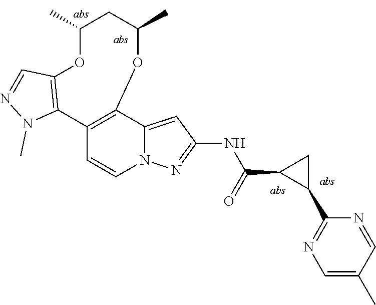 [SMILES: Cc1cnc([C@@H]2C[C@@H]2C(=O)Nc2cc3c4c(ccn3n2)-c2c(cnn2C)O[C@H](C)C[C@@H](C)O4)nc1] | LCMS-2 | 474.3 | 0.521 |
| I-133 | 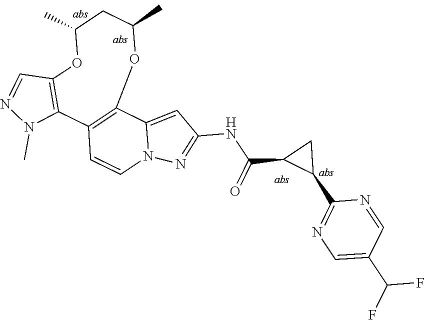 [SMILES: C[C@@H]1C[C@@H](C)Oc2c(ccn3nc(NC(=O)[C@H]4C[C@H]4c4ncc(C(F)F)cn4)cc23)-c2c(cnn2C)O1] | LCMS-1 | 510.2 | 0.48 |
| I-134 | 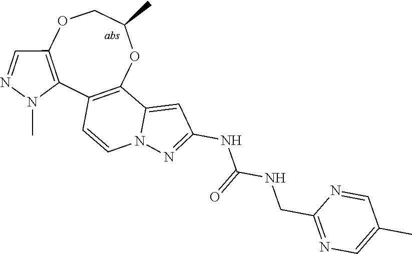 [SMILES: Cc1cnc(CNC(=O)Nc2cc3c4c(ccn3n2)-c2c(cnn2C)OC[C@@H](C)O4)nc1] | LCMS-1 | 435.3 | 0.418 |
| I-135 | 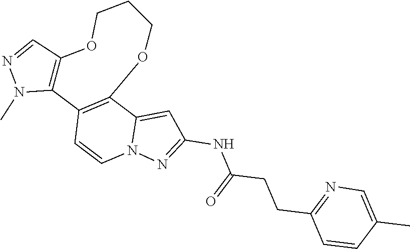 | LCMS-2 | 433.3 | 0.546 |
| I-136 | 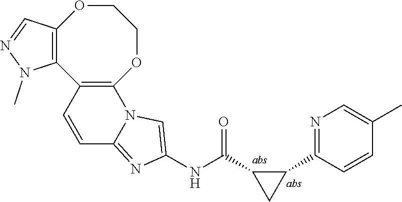 [SMILES: Cc1ccc([C@@H]2C[C@@H]2C(=O)Nc2cn3c4c(ccc3n2)-c2c(cnn2C)OCCO4)nc1] | LCMS-2 | 431.3 | 0.52 |
| I-137 | 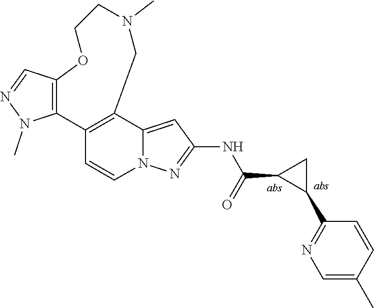 [SMILES: Cc1ccc([C@@H]2C[C@@H]2C(=O)Nc2cc3c4c(ccn3n2)-c2c(cnn2C)OCCN(C)C4)nc1] | LCMS-2 | 458.3 | 0.492 |
| I-138 | 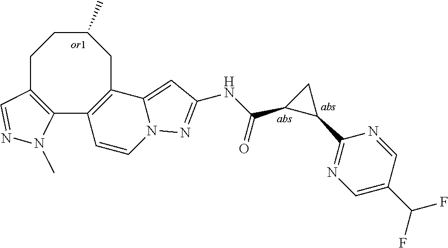 [SMILES: C[C@H]1CCc2cnn(C)c2-c2ccn3nc(NC(=O)[C@H]4C[C@H]4c4ncc(C(F)F)cn4)cc3c2C1] | LCMS-1 | 480.1 | 0.477 |
| I-139 | 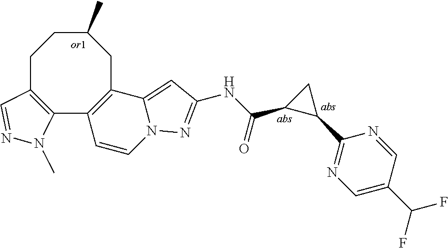 [SMILES: C[C@@H]1CCc2cnn(C)c2-c2ccn3nc(NC(=O)[C@H]4C[C@H]4c4ncc(C(F)F)cn4)cc3c2C1] | LCMS-1 | 480.1 | 0.479 |
| I-140 | 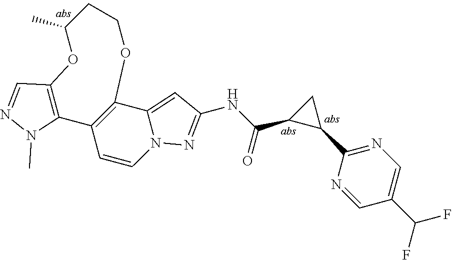 [SMILES: C[C@@H]1CCOc2c(ccn3nc(NC(=O)[C@H]4C[C@H]4c4ncc(C(F)F)cn4)cc23)-c2c(cnn2C)O1] | LCMS-1 | 496.1 | 0.508 |
| I-141 | 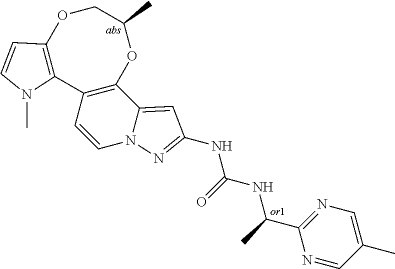 [SMILES: Cc1cnc([C@@H](C)NC(=O)Nc2cc3c4c(ccn3n2)-c2c(ccn2C)OC[C@@H](C)O4)nc1] | LCMS-1 | 449.2 | 0.444 |
| I-142 | 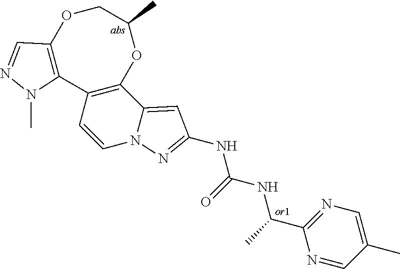 [SMILES: Cc1cnc([C@H](C)NC(=O)Nc2cc3c4c(ccn3n2)-c2c(cnn2C)OC[C@@H](C)O4)nc1] | LCMS-1 | 449.2 | 0.44 |
| I-143 | 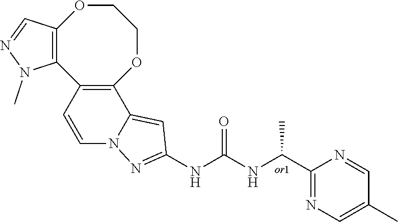 [SMILES: Cc1cnc([C@@H](C)NC(=O)Nc2cc3c4c(ccn3n2)-c2c(cnn2C)OCCO4)nc1] | LCMS-1 | 435.2 | 0.428 |
| I-144 | 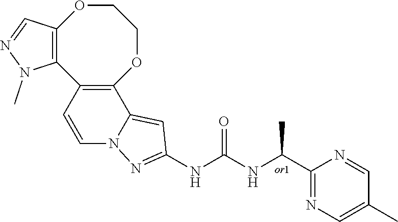 [SMILES: Cc1cnc([C@H](C)NC(=O)Nc2cc3c4c(ccn3n2)-c2c(cnn2C)OCCO4)nc1] | LCMS-1 | 435.2 | 0.429 |
| I-145 | 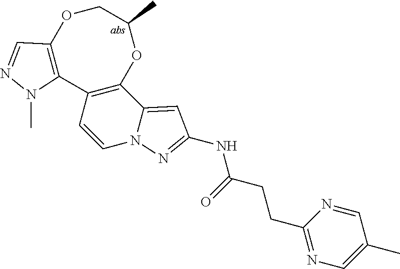 [SMILES: Cc1cnc(CCC(=O)Nc2cc3c4c(ccn3n2)-c2c(cnn2C)OC[C@@H](C)O4)nc1] | LCMS-1 | 434.2 | 0.425 |
| I-146 | 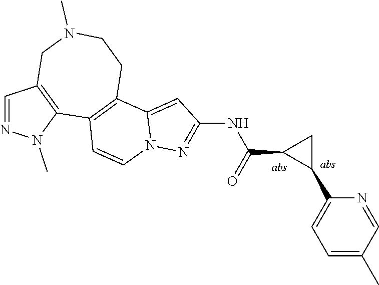 [SMILES: Cc1ccc([C@@H]2C[C@@H]2C(=O)Nc2cc3c4c(ccn3n2)-c2c(cnn2C)CN(C)CC4)nc1] | LCMS-2 | 442.3 | 0.514 |
| I-147 | 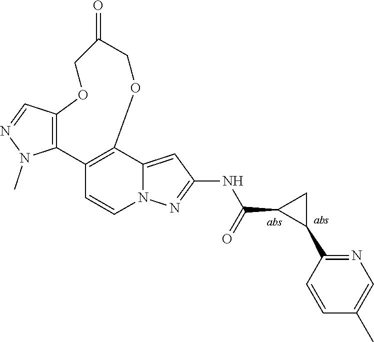 [SMILES: Cc1ccc([C@@H]2C[C@@H]2C(=O)Nc2cc3c4c(ccn3n2)-c2c(cnn2C)OCC(=O)CO4)nc1] | LCMS-2 | 459.3 | 0.52 |
| I-148 | 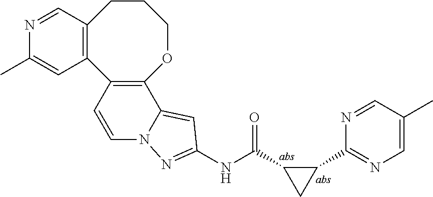 | LCMS-1 | 441.3 | 0.377 |
| I-149 | 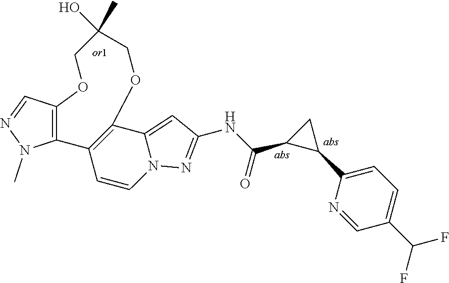 [SMILES: Cn1ncc2c1-c1ccn3nc(NC(=O)[C@H]4C[C@H]4c4ccc(C(F)F)cn4)cc3c1OC[C@@](C)(O)CO2] | LCMS-1 | 511.3 | 0.428 |
| I-150 | 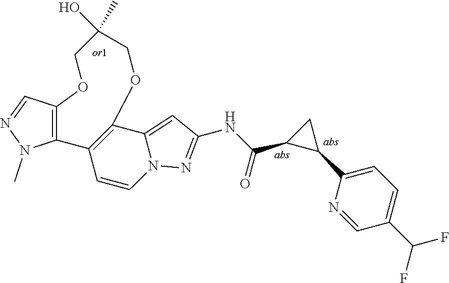 [SMILES: Cn1ncc2c1-c1ccn3nc(NC(=O)[C@H]4C[C@H]4c4ccc(C(F)F)cn4)cc3c1OC[C@](C)(O)CO2] | LCMS-1 | 511.3 | 0.425 |
| I-151 | 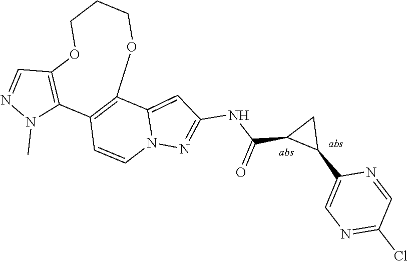 [SMILES: Cn1ncc2c1-c1ccn3nc(NC(=O)[C@H]4C[C@H]4c4cnc(Cl)cn4)cc3c1OCCCO2] | LCMS-2 | 466.2 | 0.52 |
| I-152 | 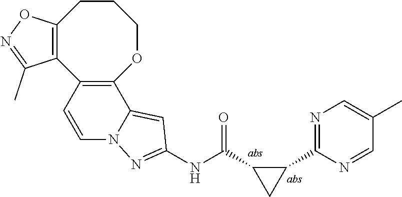 [SMILES: Cc1cnc([C@@H]2C[C@@H]2C(=O)Nc2cc3c4c(ccn3n2)-c2c(C)noc2CCCO4)nc1] | LCMS-1 | 431.2 | 0.452 |
| I-153 | 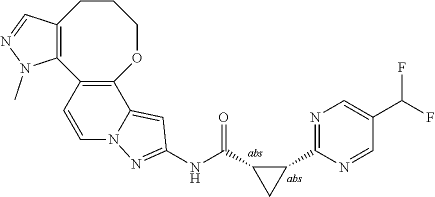 [SMILES: Cn1ncc2c1-c1ccn3nc(NC(=O)[C@H]4C[C@H]4c4ncc(C(F)F)cn4)cc3c1OCCC2] | LCMS-1 | 466.3 | 0.445 |
| I-154 | 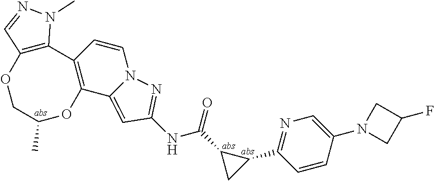 [SMILES: C[C@@H]1COc2cnn(C)c2-c2ccn3nc(NC(=O)[C@H]4C[C@H]4c4ccc(N5CC(F)C5)cn4)cc3c2O1] | LCMS-2 | 504.2 | 0.555 |
| I-155 | 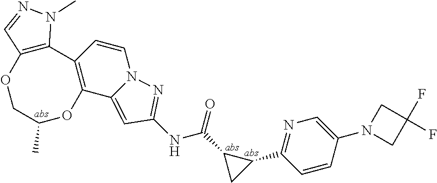 [SMILES: C[C@@H]1COc2cnn(C)c2-c2ccn3nc(NC(=O)[C@H]4C[C@H]4c4ccc(N5CC(F)(F)C5)cn4)cc3c2O1] | LCMS-2 | 522.1 | 0.556 |
| I-156 | 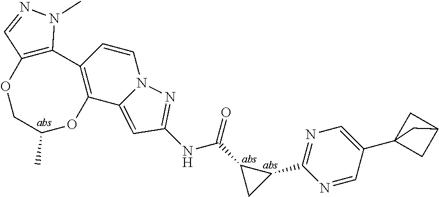 [SMILES: C[C@@H]1COc2cnn(C)c2-c2ccn3nc(NC(=O)[C@H]4C[C@H]4c4ncc(C56CC(C5)C6)cn4)cc3c2O1] | LCMS-1 | 498.3 | 0.494 |
| I-157 | 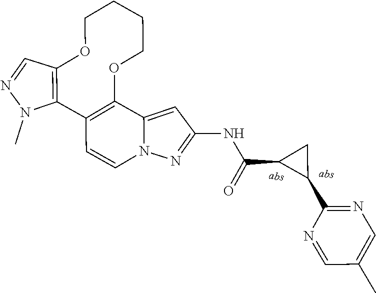 [SMILES: Cc1cnc([C@@H]2C[C@@H]2C(=O)Nc2cc3c4c(ccn3n2)-c2c(cnn2C)OCCCCO4)nc1] | LCMS-1 | 460.2 | 0.434 |
| I-158 | 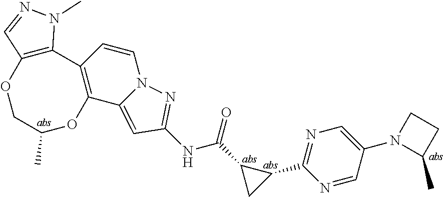 [SMILES: C[C@@H]1COc2cnn(C)c2-c2ccn3nc(NC(=O)[C@H]4C[C@H]4c4ncc(N5CC[C@H]5C)cn4)cc3c2O1] | LCMS-1 | 501.2 | 0.444 |
| I-159 | 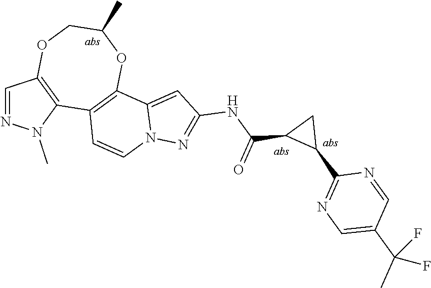 [SMILES: C[C@@H]1COc2cnn(C)c2-c2ccn3nc(NC(=O)[C@H]4C[C@H]4c4ncc(C(C)(F)F)cn4)cc3c2O1] | LCMS-1 | 496.2 | 0.474 |
| I-160 | 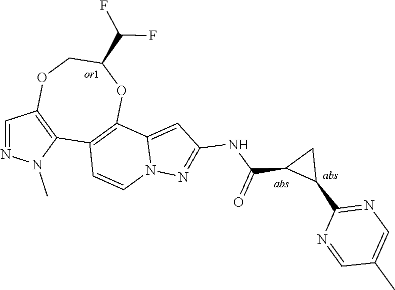 [SMILES: Cc1cnc([C@@H]2C[C@@H]2C(=O)Nc2cc3c4c(ccn3n2)-c2c(cnn2C)OC[C@@H](C(F)F)O4)nc1] | LCMS-1 | 482.2 | 0.445 |
| I-161 | 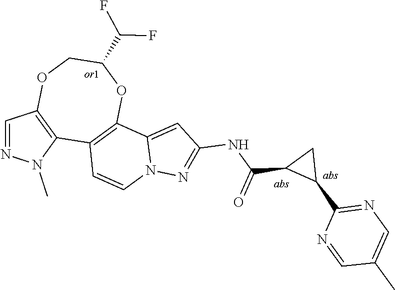 [SMILES: Cc1cnc([C@@H]2C[C@@H]2C(=O)Nc2cc3c4c(ccn3n2)-c2c(cnn2C)OC[C@H](C(F)F)O4)nc1] | LCMS-1 | 482.2 | 0.448 |
| I-162 | 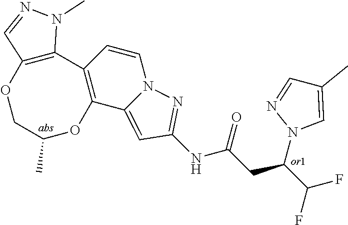 [SMILES: Cc1cnn([C@H](CC(=O)Nc2cc3c4c(ccn3n2)-c2c(cnn2C)OC[C@@H](C)O4)C(F)F)c1] | LCMS-1 | 472.2 | 0.492 |
| I-163 | 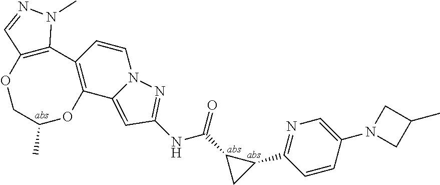 [SMILES: CC1CN(c2ccc([C@@H]3C[C@@H]3C(=O)Nc3cc4c5c(ccn4n3)-c3c(cnn3C)OC[C@@H](C)O5)nc2)C1] | LCMS-2 | 500.3 | 0.603 |
| I-164 | 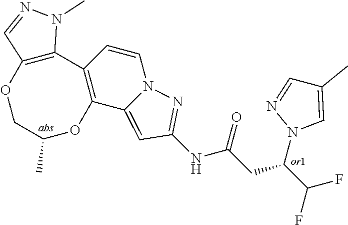 [SMILES: Cc1cnn([C@@H](CC(=O)Nc2cc3c4c(ccn3n2)-c2c(cnn2C)OC[C@@H](C)O4)C(F)F)c1] | LCMS-1 | 472.3 | 0.495 |
| I-165 | 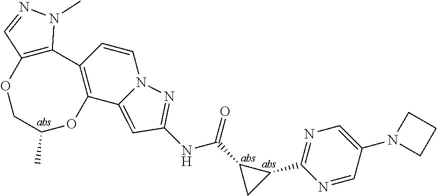 [SMILES: C[C@@H]1COc2cnn(C)c2-c2ccn3nc(NC(=O)[C@H]4C[C@H]4c4ncc(N5CCC5)cn4)cc3c2O1] | LCMS-1 | 487.2 | 0.436 |
| I-166 | 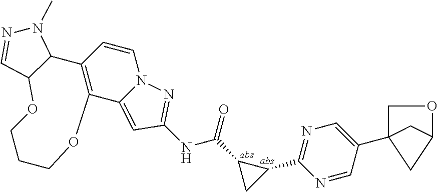 [SMILES: CN1N=CC2OCCCOc3c(ccn4nc(NC(=O)[C@H]5C[C@H]5c5ncc(C67COC(C6)C7)cn5)cc34)C21] | LCMS-1 | 514.1 | 0.462 |
| I-167 | 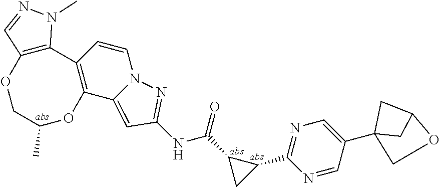 [SMILES: C[C@@H]1COc2cnn(C)c2-c2ccn3nc(NC(=O)[C@H]4C[C@H]4c4ncc(C56COC(C5)C6)cn4)cc3c2O1] | LCMS-1 | 514.1 | 0.462 |
| I-168 | 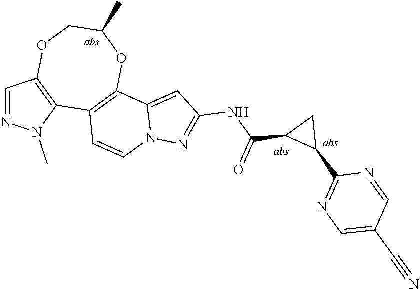 [SMILES: C[C@@H]1COc2cnn(C)c2-c2ccn3nc(NC(=O)[C@H]4C[C@H]4c4ncc(C#N)cn4)cc3c2O1] | LCMS-1 | 457.2 | 0.464 |
| I-169 | 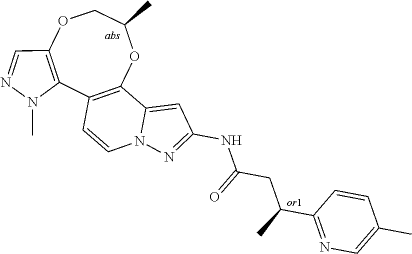 [SMILES: Cc1ccc([C@@H](C)CC(=O)Nc2cc3c4c(ccn3n2)-c2c(cnn2C)OC[C@@H](C)O4)nc1] | LCMS-1 | 447.2 | 0.393 |
| I-170 | 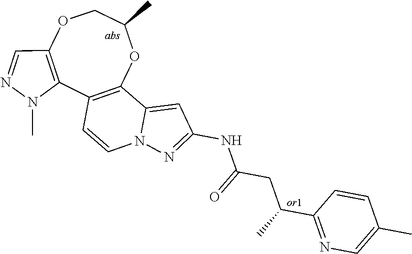 [SMILES: Cc1ccc([C@H](C)CC(=O)Nc2cc3c4c(ccn3n2)-c2c(cnn2C)OC[C@@H](C)O4)nc1] | LCMS-1 | 447.2 | 0.397 |
| I-171 | 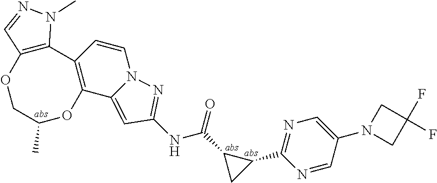 [SMILES: C[C@@H]1COc2cnn(C)c2-c2ccn3nc(NC(=O)[C@H]4C[C@H]4c4ncc(N5CC(F)(F)C5)cn4)cc3c2O1] | LCMS-1 | 523.3 | 0.461 |
| I-172 | 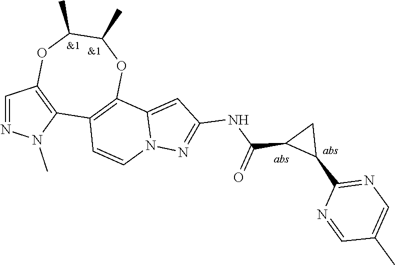 [SMILES: Cc1cnc([C@@H]2C[C@@H]2C(=O)Nc2cc3c4c(ccn3n2)-c2c(cnn2C)O[C@@H](C)[C@@H](C)O4)nc1] | LCMS-1 | 460.1 | 0.469 |
| I-173 | 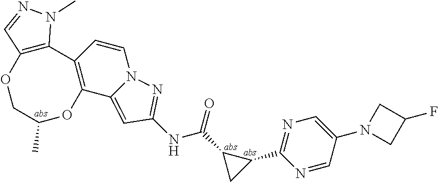 [SMILES: C[C@@H]1COc2cnn(C)c2-c2ccn3nc(NC(=O)[C@H]4C[C@H]4c4ncc(N5CC(F)C5)cn4)cc3c2O1] | LCMS-1 | 505.3 | 0.418 |
| I-174 | 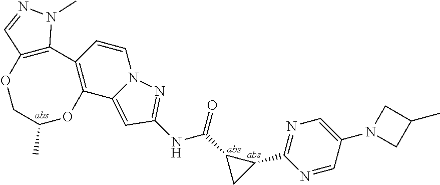 [SMILES: CC1CN(c2cnc([C@@H]3C[C@@H]3C(=O)Nc3cc4c5c(ccn4n3)-c3c(cnn3C)OC[C@@H](C)O5)nc2)C1] | LCMS-1 | 501.3 | 0.448 |
| I-175 | 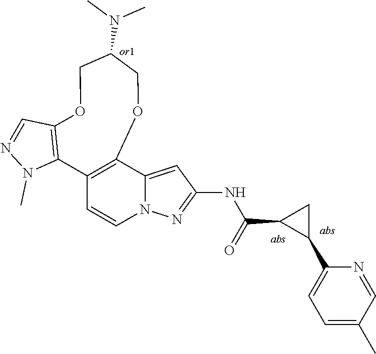 [SMILES: Cc1ccc([C@@H]2C[C@@H]2C(=O)Nc2cc3c4c(ccn3n2)-c2c(cnn2C)OC[C@H](N(C)C)CO4)nc1] | LCMS-2 | 488.2 | 0.518 |
| I-176 | 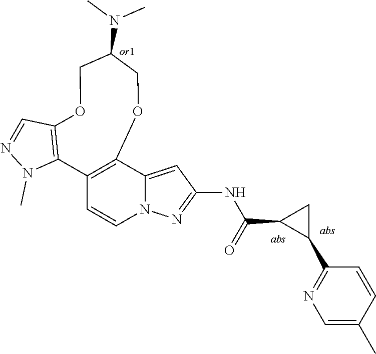 [SMILES: Cc1ccc([C@@H]2C[C@@H]2C(=O)Nc2cc3c4c(ccn3n2)-c2c(cnn2C)OC[C@@H](N(C)C)CO4)nc1] | LCMS-2 | 488.3 | 0.518 |
| I-177 | 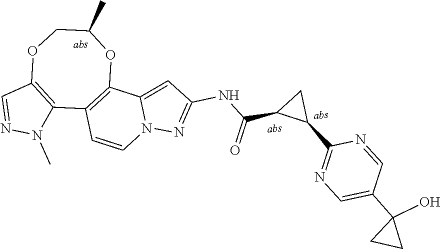 [SMILES: C[C@@H]1COc2cnn(C)c2-c2ccn3nc(NC(=O)[C@H]4C[C@H]4c4ncc(C5(O)CC5)cn4)cc3c2O1] | LCMS-1 | 488.2 | 0.43 |
| I-178 | 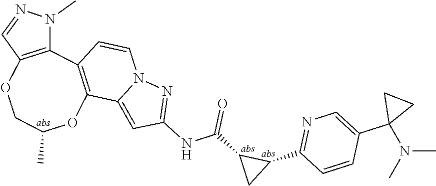 [SMILES: C[C@@H]1COc2cnn(C)c2-c2ccn3nc(NC(=O)[C@H]4C[C@H]4c4ccc(C5(N(C)C)CC5)cn4)cc3c2O1] | LCMS-1 | 514.3 | 0.381 |
| I-179 | 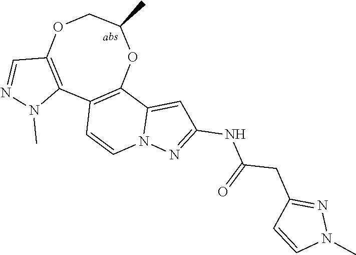 [SMILES: C[C@@H]1COc2cnn(C)c2-c2ccn3nc(NC(=O)Cc4ccn(C)n4)cc3c2O1] | LCMS-1 | 408.1 | 0.447 |
| I-180 | 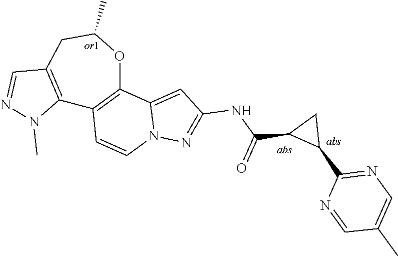 [SMILES: Cc1cnc([C@@H]2C[C@@H]2C(=O)Nc2cc3c4c(ccn3n2)-c2c(cnn2C)C[C@H](C)O4)nc1] | LCMS-1 | 430.3 | 0.439 |
| I-181 | 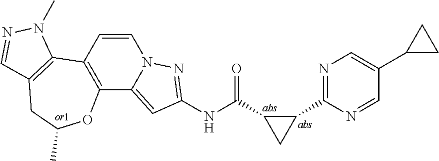 [SMILES: C[C@@H]1Cc2cnn(C)c2-c2ccn3nc(NC(=O)[C@H]4C[C@H]4c4ncc(C5CC5)cn4)cc3c2O1] | LCMS-1 | 456.3 | 0.469 |
| I-182 | 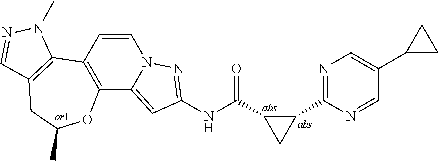 [SMILES: C[C@H]1Cc2cnn(C)c2-c2ccn3nc(NC(=O)[C@H]4C[C@H]4c4ncc(C5CC5)cn4)cc3c2O1] | LCMS-1 | 456.3 | 0.468 |
| I-183 | 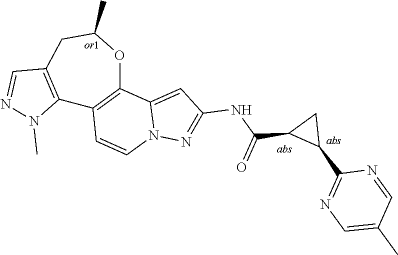 [SMILES: Cc1cnc([C@@H]2C[C@@H]2C(=O)Nc2cc3c4c(ccn3n2)-c2c(cnn2C)C[C@@H](C)O4)nc1] | LCMS-1 | 430.3 | 0.443 |
| I-184 | 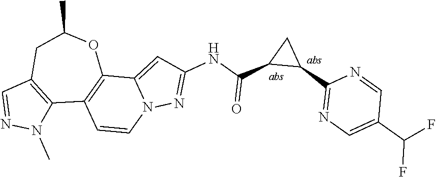 [SMILES: C[C@@H]1Cc2cnn(C)c2-c2ccn3nc(NC(=O)[C@H]4C[C@H]4c4ncc(C(F)F)cn4)cc3c2O1] | LCMS-1 | 466.3 | 0.464 |
| I-185 | 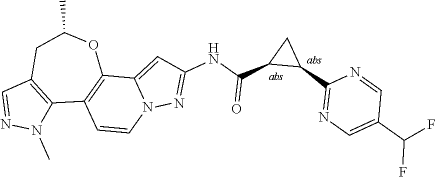 [SMILES: C[C@H]1Cc2cnn(C)c2-c2ccn3nc(NC(=O)[C@H]4C[C@H]4c4ncc(C(F)F)cn4)cc3c2O1] | LCMS-1 | 466.2 | 0.466 |
| I-186 | 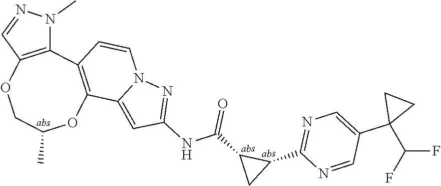 [SMILES: C[C@@H]1COc2cnn(C)c2-c2ccn3nc(NC(=O)[C@H]4C[C@H]4c4ncc(C5(C(F)F)CC5)cn4)cc3c2O1] | LCMS-1 | 522.2 | 0.47 |
| I-187 | 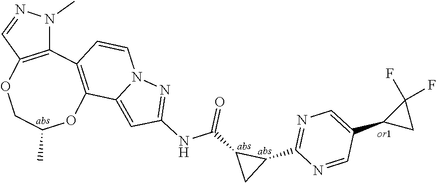 [SMILES: C[C@@H]1COc2cnn(C)c2-c2ccn3nc(NC(=O)[C@H]4C[C@H]4c4ncc([C@@H]5CC5(F)F)cn4)cc3c2O1] | LCMS-1 | 508.2 | 0.459 |
| I-188 | 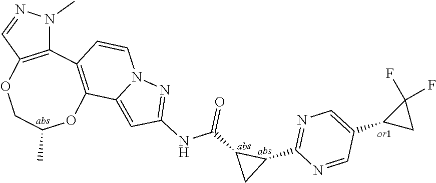 [SMILES: C[C@@H]1COc2cnn(C)c2-c2ccn3nc(NC(=O)[C@H]4C[C@H]4c4ncc([C@H]5CC5(F)F)cn4)cc3c2O1] | LCMS-1 | 508.2 | 0.459 |
| I-189 | 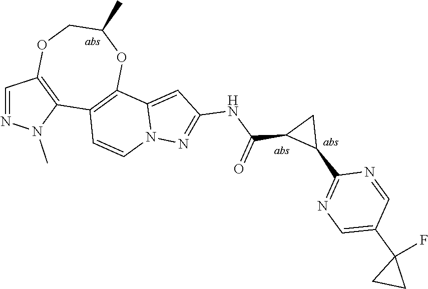 [SMILES: C[C@@H]1COc2cnn(C)c2-c2ccn3nc(NC(=O)[C@H]4C[C@H]4c4ncc(C5(F)CC5)cn4)cc3c2O1] | LCMS-1 | 490.2 | 0.473 |
| I-190 | 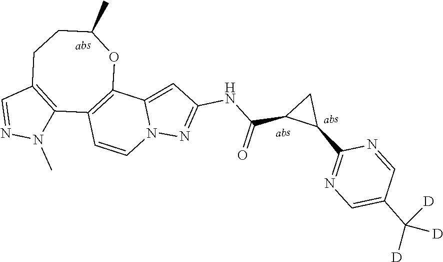 | LCMS-1 | 463.2 | 0.44 |
| I-191 | 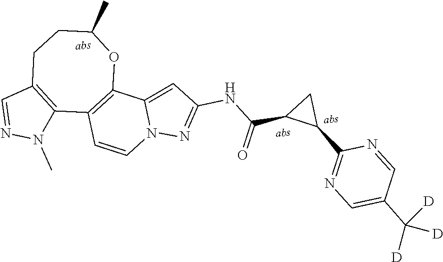 [SMILES: [2H]C([2H])([2H])c1cnc([C@@H]2C[C@@H]2C(=O)Nc2cc3c4c(ccn3n2)-c2c(cnn2C)CC[C@@H](C)O4)nc1] | LCMS-1 | 447.3 | 0.44 |
| I-192 | 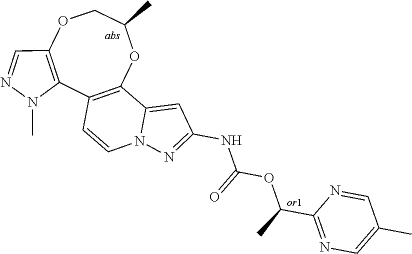 [SMILES: Cc1cnc([C@@H](C)OC(=O)Nc2cc3c4c(ccn3n2)-c2c(cnn2C)OC[C@@H](C)O4)nc1] | LCMS-1 | 450.2 | 0.466 |
| I-193 | 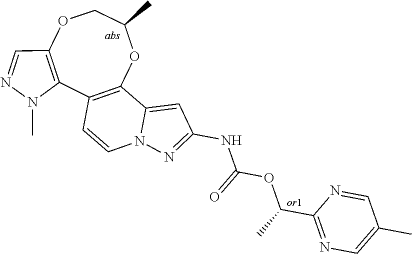 [SMILES: Cc1cnc([C@H](C)OC(=O)Nc2cc3c4c(ccn3n2)-c2c(cnn2C)OC[C@@H](C)O4)nc1] | LCMS-1 | 450.2 | 0.464 |
| I-194 | 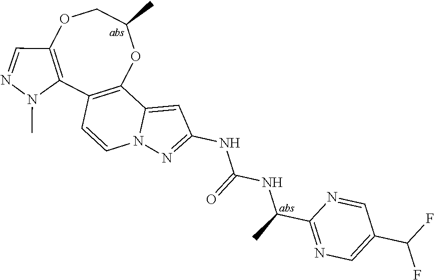 [SMILES: C[C@@H]1COc2cnn(C)c2-c2ccn3nc(NC(=O)N[C@H](C)c4ncc(C(F)F)cn4)cc3c2O1] | LCMS-1 | 485 | 0.479 |
| I-195 | 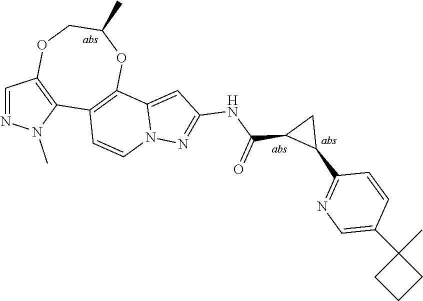 [SMILES: C[C@@H]1COc2cnn(C)c2-c2ccn3nc(NC(=O)[C@H]4C[C@H]4c4ccc(C5(C)CCC5)cn4)cc3c2O1] | LCMS-1 | 501.1 | 0.413 |
| I-196 | 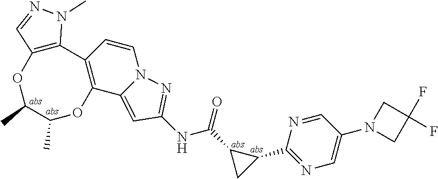 [SMILES: C[C@H]1Oc2cnn(C)c2-c2ccn3nc(NC(=O)[C@H]4C[C@H]4c4ncc(N5CC(F)(F)C5)cn4)cc3c2O[C@@H]1C] | LCMS-1 | 537.1 | 0.472 |
| I-197 | 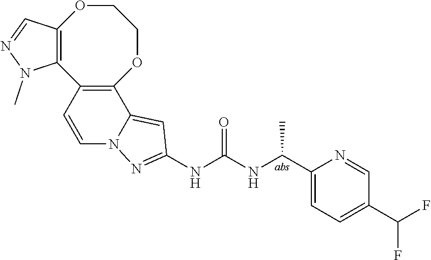 [SMILES: C[C@@H](NC(=O)Nc1cc2c3c(ccn2n1)-c1c(cnn1C)OCCO3)c1ccc(C(F)F)cn1] | LCMS-1 | 470.1 | 0.467 |
| I-198 | 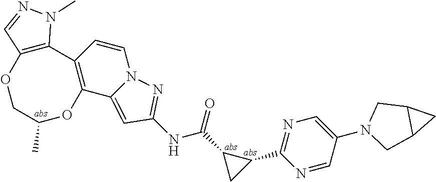 [SMILES: C[C@@H]1COc2cnn(C)c2-c2ccn3nc(NC(=O)[C@H]4C[C@H]4c4ncc(N5CC6CC6C5)cn4)cc3c2O1] | LCMS-1 | 513.3 | 0.449 |
| I-199 | 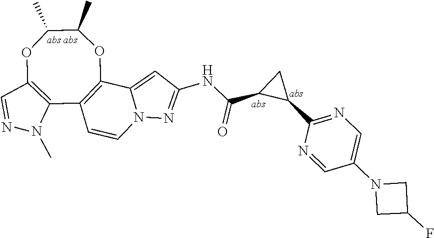 [SMILES: C[C@H]1Oc2cnn(C)c2-c2ccn3nc(NC(=O)[C@H]4C[C@H]4c4ncc(N5CC(F)C5)cn4)cc3c2O[C@@H]1C] | LCMS-1 | 519.1 | 0.458 |
| I-200 | 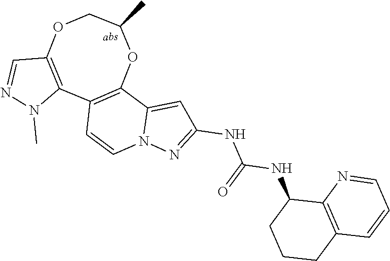 [SMILES: C[C@@H]1COc2cnn(C)c2-c2ccn3nc(NC(=O)N[C@@H]4CCCc5cccnc54)cc3c2O1] | LCMS-1 | 460.3 | 0.429 |
| I-201 | 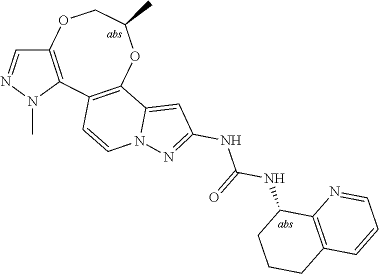 [SMILES: C[C@@H]1COc2cnn(C)c2-c2ccn3nc(NC(=O)N[C@H]4CCCc5cccnc54)cc3c2O1] | LCMS-1 | 460.3 | 0.428 |
| I-202 | 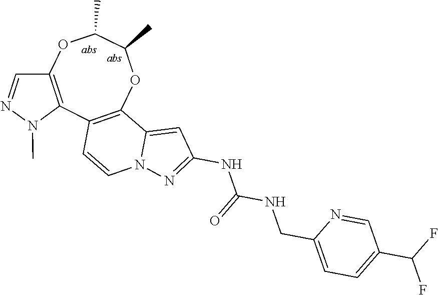 [SMILES: C[C@H]1Oc2cnn(C)c2-c2ccn3nc(NC(=O)NCc4ccc(C(F)F)cn4)cc3c2O[C@@H]1C] | LCMS-1 | 484.2 | 0.472 |
| I-203 | 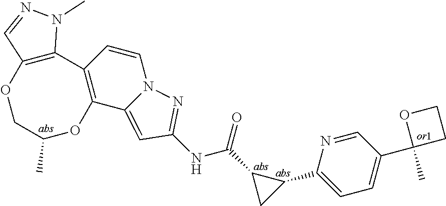 [SMILES: C[C@@H]1COc2cnn(C)c2-c2ccn3nc(NC(=O)[C@H]4C[C@H]4c4ccc([C@]5(C)CCO5)cn4)cc3c2O1] | LCMS-2 | 501.3 | 0.542 |
| I-204 | 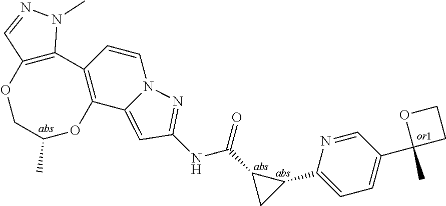 [SMILES: C[C@@H]1COc2cnn(C)c2-c2ccn3nc(NC(=O)[C@H]4C[C@H]4c4ccc([C@@]5(C)CCO5)cn4)cc3c2O1] | LCMS-2 | 501.3 | 0.544 |
| I-205 | 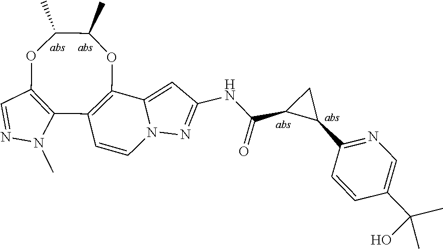 [SMILES: C[C@H]1Oc2cnn(C)c2-c2ccn3nc(NC(=O)[C@H]4C[C@H]4c4ccc(C(C)(C)O)cn4)cc3c2O[C@@H]1C] | LCMS-1 | 503.2 | 0.416 |
| I-206 | 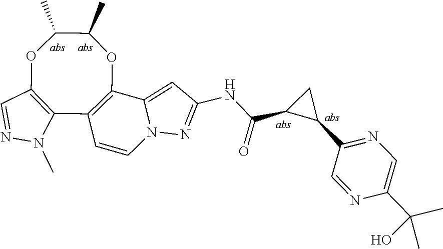 [SMILES: C[C@H]1Oc2cnn(C)c2-c2ccn3nc(NC(=O)[C@H]4C[C@H]4c4cnc(C(C)(C)O)cn4)cc3c2O[C@@H]1C] | LCMS-1 | 504.2 | 0.457 |
| I-207 | 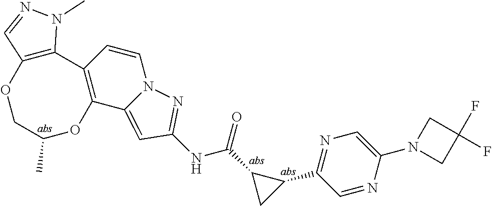 [SMILES: C[C@@H]1COc2cnn(C)c2-c2ccn3nc(NC(=O)[C@H]4C[C@H]4c4cnc(N5CC(F)(F)C5)cn4)cc3c2O1] | LCMS-1 | 523.2 | 0.48 |
| I-208 | 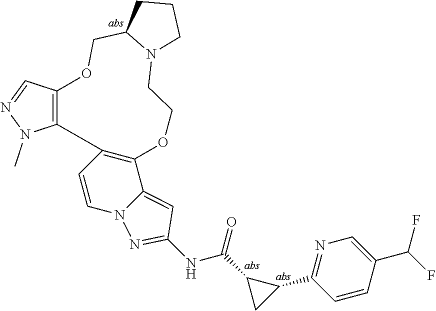 [SMILES: Cn1ncc2c1-c1ccn3nc(NC(=O)[C@H]4C[C@H]4c4ccc(C(F)F)cn4)cc3c1OCCN1CCC[C@@H]1CO2] | LCMS-1 | 550.2 | 0.398 |
| I-209 | 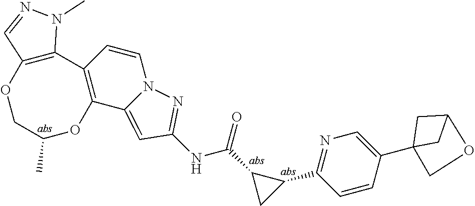 [SMILES: C[C@@H]1COc2cnn(C)c2-c2ccn3nc(NC(=O)[C@H]4C[C@H]4c4ccc(C56COC(C5)C6)cn4)cc3c2O1] | LCMS-1 | 513.1 | 0.41 |
| I-210 | 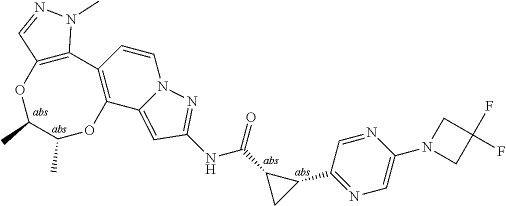 [SMILES: C[C@H]1Oc2cnn(C)c2-c2ccn3nc(NC(=O)[C@H]4C[C@H]4c4cnc(N5CC(F)(F)C5)cn4)cc3c2O[C@@H]1C] | LCMS-2 | 537.3 | 0.584 |
| I-211 | 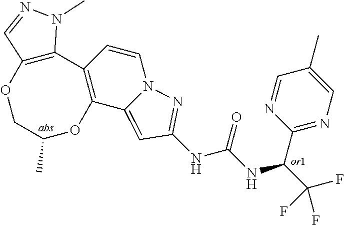 [SMILES: Cc1cnc([C@H](NC(=O)Nc2cc3c4c(ccn3n2)-c2c(cnn2C)OC[C@@H](C)O4)C(F)(F)F)nc1] | LCMS-1 | 503.2 | 0.527 |
| I-212 | 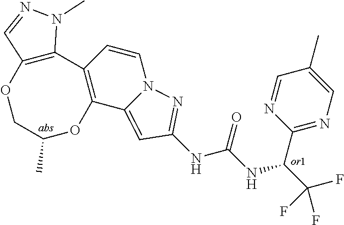 [SMILES: Cc1cnc([C@@H](NC(=O)Nc2cc3c4c(ccn3n2)-c2c(cnn2C)OC[C@@H](C)O4)C(F)(F)F)nc1] | LCMS-1 | 503.2 | 0.532 |
| I-213 | 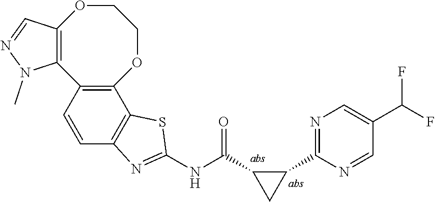 [SMILES: Cn1ncc2c1-c1ccc3nc(NC(=O)[C@H]4C[C@H]4c4ncc(C(F)F)cn4)sc3c1OCCO2] | LCMS-1 | 485.3 | 0.473 |
| I-214 | 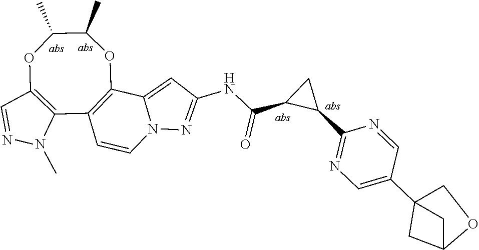 [SMILES: C[C@H]1Oc2cnn(C)c2-c2ccn3nc(NC(=O)[C@H]4C[C@H]4c4ncc(C56COC(C5)C6)cn4)cc3c2O[C@@H]1C] | LCMS-1 | 528.1 | 0.447 |
| I-215 | 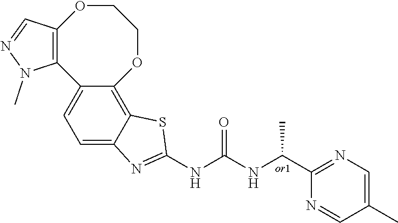 [SMILES: Cc1cnc([C@@H](C)NC(=O)Nc2nc3ccc4c(c3s2)OCCOc2cnn(C)c2-4)nc1] | LCMS-1 | 452.2 | 0.462 |
| I-216 | 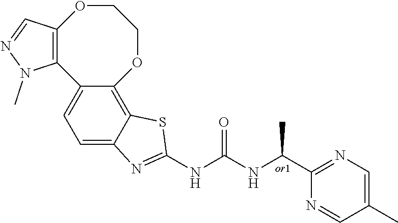 [SMILES: Cc1cnc([C@H](C)NC(=O)Nc2nc3ccc4c(c3s2)OCCOc2cnn(C)c2-4)nc1] | LCMS-1 | 452.3 | 0.464 |
| I-217 | 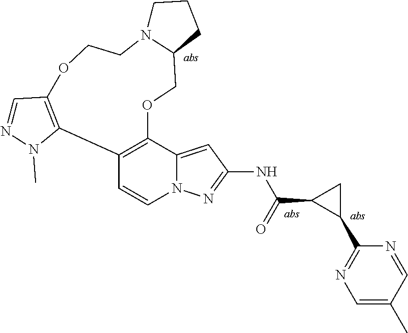 [SMILES: Cc1cnc([C@@H]2C[C@@H]2C(=O)Nc2cc3c4c(ccn3n2)-c2c(cnn2C)OCCN2CCC[C@H]2CO4)nc1] | LCMS-1 | 515.3 | 0.381 |
| I-218 | 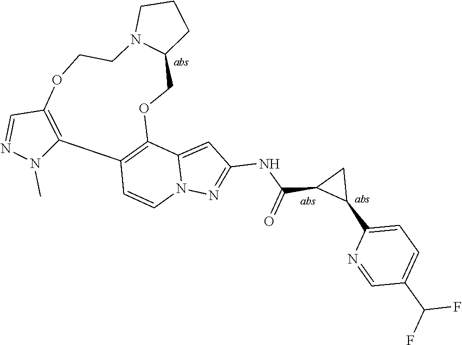 [SMILES: Cn1ncc2c1-c1ccn3nc(NC(=O)[C@H]4C[C@H]4c4ccc(C(F)F)cn4)cc3c1OC[C@@H]1CCCN1CCO2] | LCMS-1 | 550.2 | 0.394 |
| I-219 | 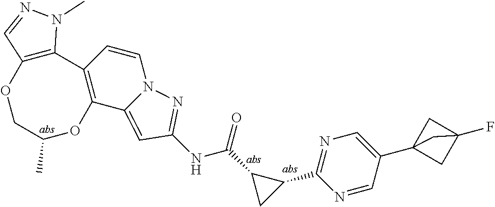 [SMILES: C[C@@H]1COc2cnn(C)c2-c2ccn3nc(NC(=O)[C@H]4C[C@H]4c4ncc(C56CC(F)(C5)C6)cn4)cc3c2O1] | LCMS-1 | 516.3 | 0.484 |
| I-220 | 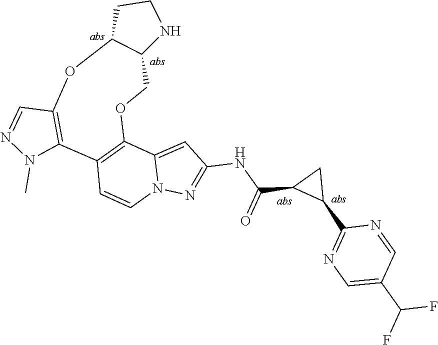 [SMILES: Cn1ncc2c1-c1ccn3nc(NC(=O)[C@H]4C[C@H]4c4ncc(C(F)F)cn4)cc3c1OC[C@H]1NCC[C@H]1O2] | LCMS-1 | 523.2 | 0.382 |
| I-221 | 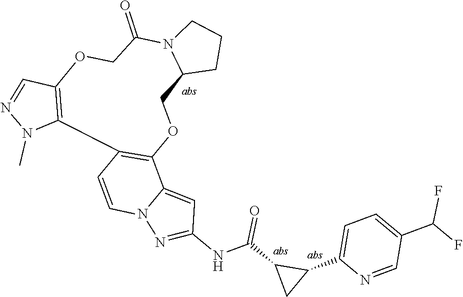 [SMILES: Cn1ncc2c1-c1ccn3nc(NC(=O)[C@H]4C[C@H]4c4ccc(C(F)F)cn4)cc3c1OC[C@@H]1CCCN1C(=O)CO2] | LCMS-1 | 564.3 | 0.421 |
| I-222 | 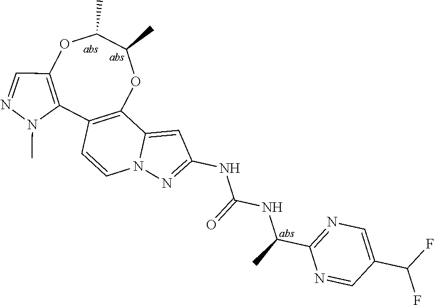 [SMILES: C[C@@H](NC(=O)Nc1cc2c3c(ccn2n1)-c1c(cnn1C)O[C@H](C)[C@@H](C)O3)c1ncc(C(F)F)cn1] | LCMS-1 | 499.2 | 0.482 |
| I-223 | 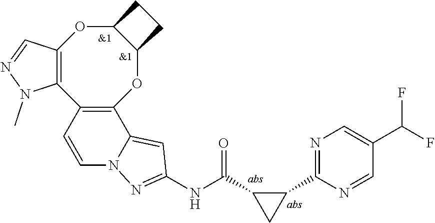 [SMILES: Cn1ncc2c1-c1ccn3nc(NC(=O)[C@H]4C[C@H]4c4ncc(C(F)F)cn4)cc3c1O[C@@H]1CC[C@@H]1O2] | LCMS-1 | 494.1 | 0.501 |
| I-224 | 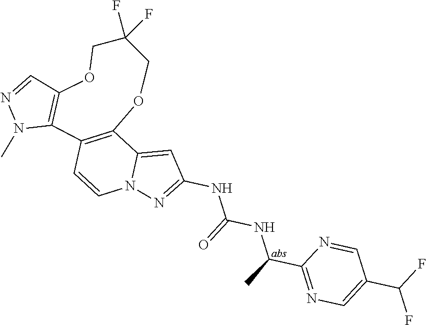 [SMILES: C[C@@H](NC(=O)Nc1cc2c3c(ccn2n1)-c1c(cnn1C)OCC(F)(F)CO3)c1ncc(C(F)F)cn1] | LCMS-1 | 521.3 | 0.49 |
| I-225 | 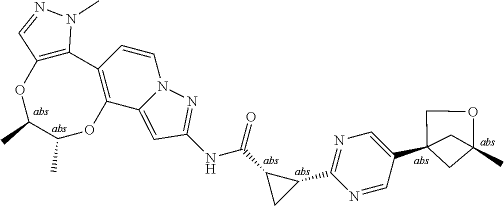 | LCMS-1 | 542.1 | 0.461 |
| I-226 | 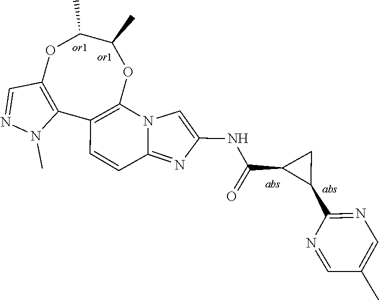 [SMILES: Cc1cnc([C@@H]2C[C@@H]2C(=O)Nc2cn3c4c(ccc3n2)-c2c(cnn2C)O[C@H](C)[C@@H](C)O4)nc1] | LCMS-1 | 460.2 | 0.421 |
| I-227 | 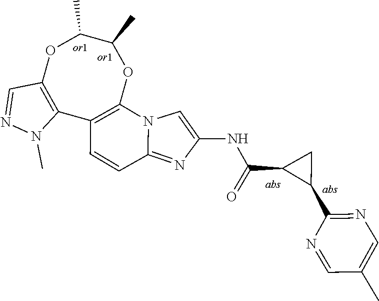 [SMILES: Cc1cnc([C@@H]2C[C@@H]2C(=O)Nc2cn3c4c(ccc3n2)-c2c(cnn2C)O[C@H](C)[C@@H](C)O4)nc1] | LCMS-1 | 460.2 | 0.418 |
| I-228 | 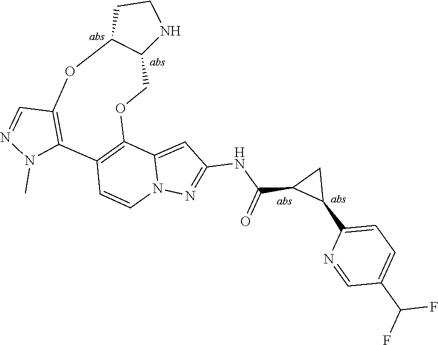 [SMILES: Cn1ncc2c1-c1ccn3nc(NC(=O)[C@H]4C[C@H]4c4ccc(C(F)F)cn4)cc3c1OC[C@H]1NCC[C@H]1O2] | LCMS-1 | 522.2 | 0.382 |
| I-229 | 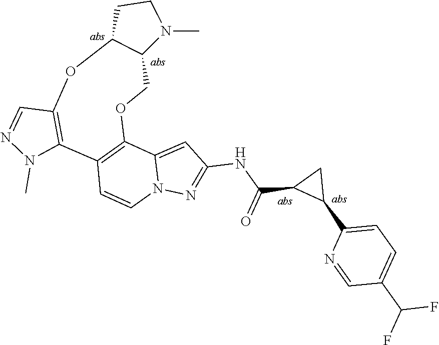 [SMILES: CN1CC[C@H]2Oc3cnn(C)c3-c3ccn4nc(NC(=O)[C@H]5C[C@H]5c5ccc(C(F)F)cn5)cc4c3OC[C@H]21] | LCMS-1 | 536.3 | 0.377 |
| I-230 | 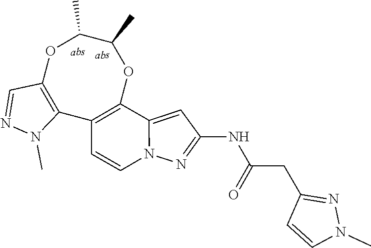 [SMILES: C[C@H]1Oc2cnn(C)c2-c2ccn3nc(NC(=O)Cc4ccn(C)n4)cc3c2O[C@@H]1C] | LCMS-1 | 422.2 | 0.452 |
| I-231 | 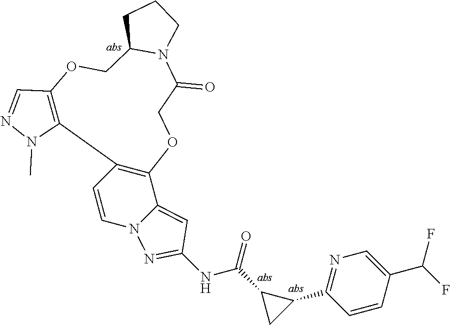 [SMILES: Cn1ncc2c1-c1ccn3nc(NC(=O)[C@H]4C[C@H]4c4ccc(C(F)F)cn4)cc3c1OCC(=O)N1CCC[C@@H]1CO2] | LCMS-1 | 564.3 | 0.42 |
| I-232 | 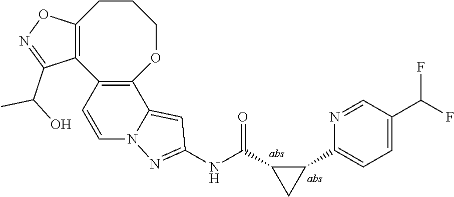 [SMILES: CC(O)c1noc2c1-c1ccn3nc(NC(=O)[C@H]4C[C@H]4c4ccc(C(F)F)cn4)cc3c1OCCC2] | LCMS-1 | 496 | 0.483 |
| I-233 | 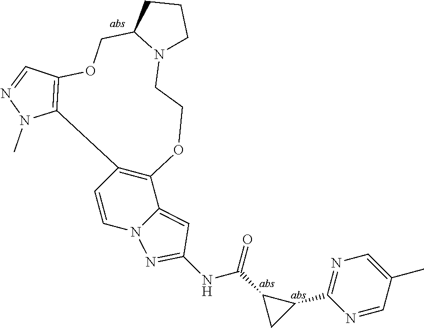 [SMILES: Cc1cnc([C@@H]2C[C@@H]2C(=O)Nc2cc3c4c(ccn3n2)-c2c(cnn2C)OC[C@H]2CCCN2CCO4)nc1] | LCMS-1 | 515.3 | 0.372 |
| I-234 | 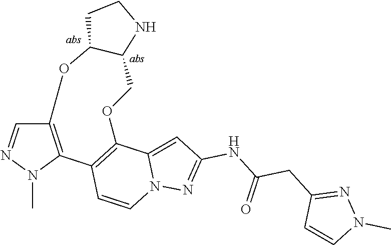 [SMILES: Cn1ccc(CC(=O)Nc2cc3c4c(ccn3n2)-c2c(cnn2C)O[C@@H]2CCN[C@@H]2CO4)n1] | LCMS-1 | 449.2 | 0.361 |
| I-235 | 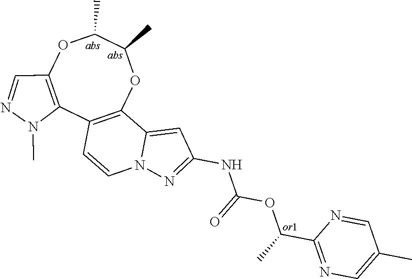 [SMILES: Cc1cnc([C@H](C)OC(=O)Nc2cc3c4c(ccn3n2)-c2c(cnn2C)O[C@H](C)[C@@H](C)O4)nc1] | LCMS-2 | 462.3 | 0.553 |
| I-236 | 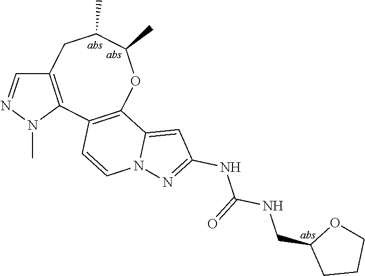 [SMILES: C[C@H]1Cc2cnn(C)c2-c2ccn3nc(NC(=O)NC[C@@H]4CCCO4)cc3c2O[C@@H]1C] | LCMS-2 | 427.2 | 0.54 |
| I-237 | 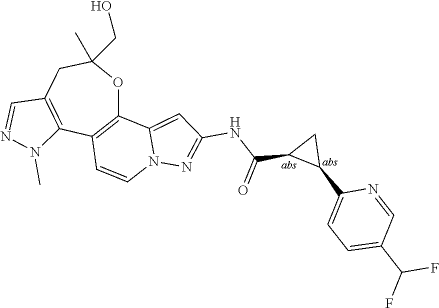 [SMILES: Cn1ncc2c1-c1ccn3nc(NC(=O)[C@H]4C[C@H]4c4ccc(C(F)F)cn4)cc3c1OC(C)(CO)C2] | LCMS-1 | 495.3 | 0.428 |
| I-238 | 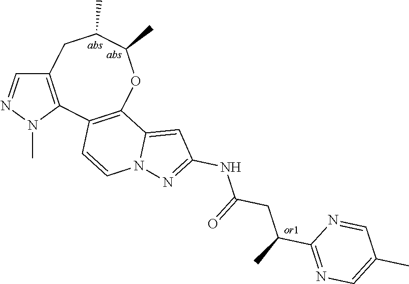 [SMILES: Cc1cnc([C@@H](C)CC(=O)Nc2cc3c4c(ccn3n2)-c2c(cnn2C)C[C@H](C)[C@@H](C)O4)nc1] | LCMS-1 | 462.3 | 0.469 |
| I-239 | 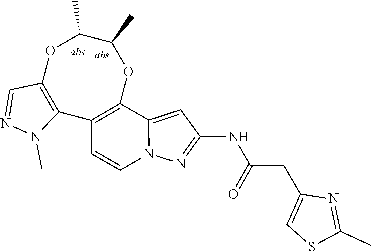 [SMILES: Cc1nc(CC(=O)Nc2cc3c4c(ccn3n2)-c2c(cnn2C)O[C@H](C)[C@@H](C)O4)cs1] | LCMS-1 | 439.2 | 0.465 |
| I-240 | 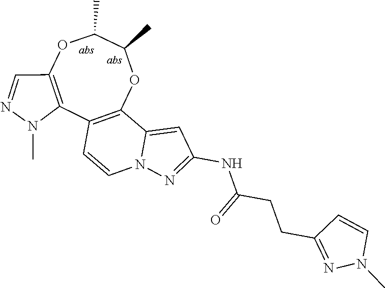 [SMILES: C[C@H]1Oc2cnn(C)c2-c2ccn3nc(NC(=O)CCc4ccn(C)n4)cc3c2O[C@@H]1C] | LCMS-1 | 436.2 | 0.452 |
| I-241 | 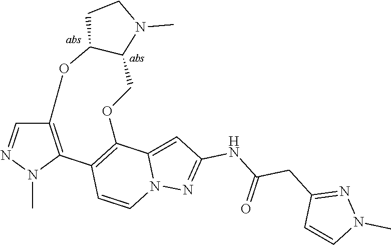 [SMILES: CN1CC[C@H]2Oc3cnn(C)c3-c3ccn4nc(NC(=O)Cc5ccn(C)n5)cc4c3OC[C@H]21] | LCMS-1 | 463.2 | 0.364 |
| I-242 | 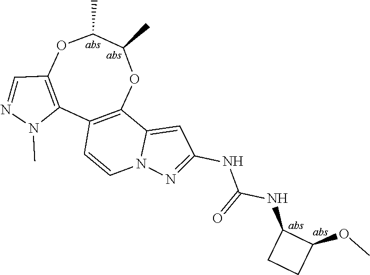 [SMILES: CO[C@H]1CC[C@H]1NC(=O)Nc1cc2c3c(ccn2n1)-c1c(cnn1C)O[C@H](C)[C@@H](C)O3] | LCMS-1 | 427.2 | 0.479 |
| I-243 | 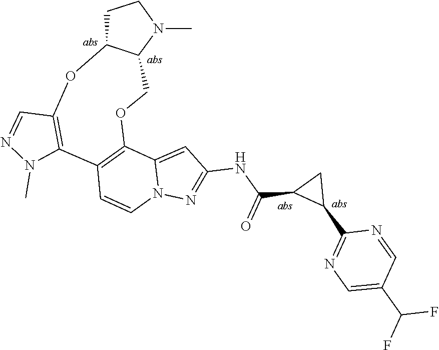 [SMILES: CN1CC[C@H]2Oc3cnn(C)c3-c3ccn4nc(NC(=O)[C@H]5C[C@H]5c5ncc(C(F)F)cn5)cc4c3OC[C@H]21] | LCMS-1 | 537.3 | 0.382 |
| I-244 | 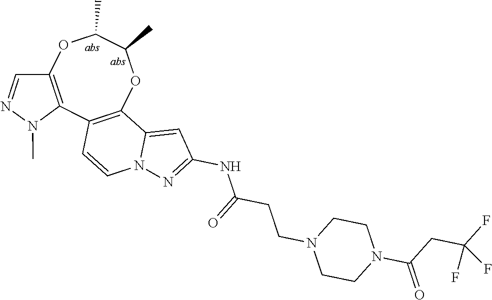 [SMILES: C[C@H]1Oc2cnn(C)c2-c2ccn3nc(NC(=O)CCN4CCN(C(=O)CC(F)(F)F)CC4)cc3c2O[C@@H]1C] | LCMS-1 | 550.4 | 0.423 |
| I-245 | 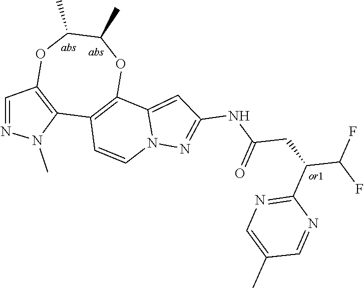 [SMILES: Cc1cnc([C@H](CC(=O)Nc2cc3c4c(ccn3n2)-c2c(cnn2C)O[C@H](C)[C@@H](C)O4)C(F)F)nc1] | LCMS-1 | 498.2 | 0.503 |
| I-246 | 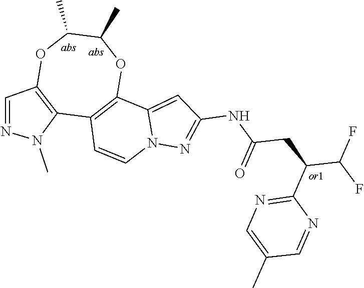 [SMILES: Cc1cnc([C@@H](CC(=O)Nc2cc3c4c(ccn3n2)-c2c(cnn2C)O[C@H](C)[C@@H](C)O4)C(F)F)nc1] | LCMS-1 | 498.2 | 0.507 |
| I-247 | 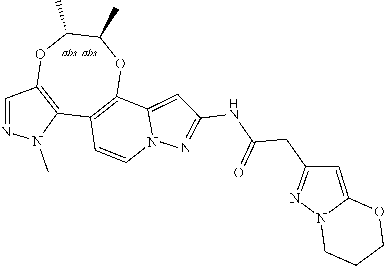 [SMILES: C[C@H]1Oc2cnn(C)c2-c2ccn3nc(NC(=O)Cc4cc5n(n4)CCCO5)cc3c2O[C@@H]1C] | LCMS-1 | 464.2 | 0.454 |
| I-248 | 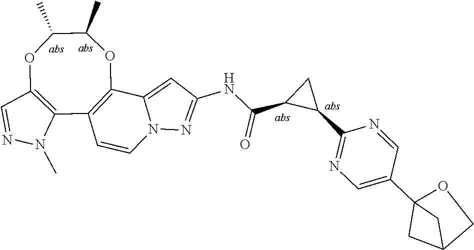 [SMILES: C[C@H]1Oc2cnn(C)c2-c2ccn3nc(NC(=O)[C@H]4C[C@H]4c4ncc(C56CC(CO5)C6)cn4)cc3c2O[C@@H]1C] | LCMS-1 | 528.3 | 0.467 |
| I-249 | 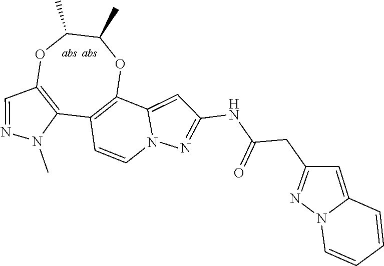 [SMILES: C[C@H]1Oc2cnn(C)c2-c2ccn3nc(NC(=O)Cc4cc5ccccn5n4)cc3c2O[C@@H]1C] | LCMS-1 | 458.2 | 0.491 |
| I-250 | 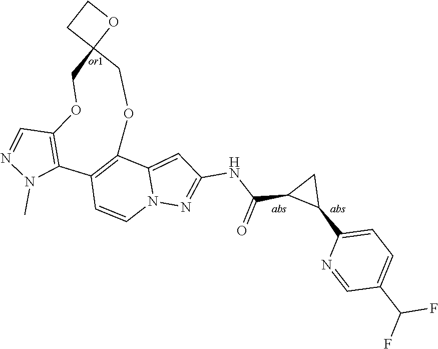 [SMILES: Cn1ncc2c1-c1ccn3nc(NC(=O)[C@H]4C[C@H]4c4ccc(C(F)F)cn4)cc3c1OC[C@@]1(CCO1)CO2] | LCMS-1 | 523.2 | 0.447 |
| I-251 | 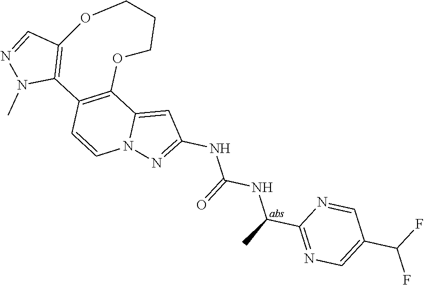 [SMILES: C[C@@H](NC(=O)Nc1cc2c3c(ccn2n1)-c1c(cnn1C)OCCCO3)c1ncc(C(F)F)cn1] | LCMS-1 | 485 | 0.454 |
| I-252 | 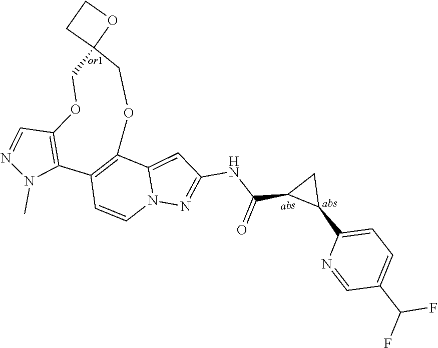 [SMILES: Cn1ncc2c1-c1ccn3nc(NC(=O)[C@H]4C[C@H]4c4ccc(C(F)F)cn4)cc3c1OC[C@]1(CCO1)CO2] | LCMS-1 | 523.2 | 0.451 |
| I-253 | 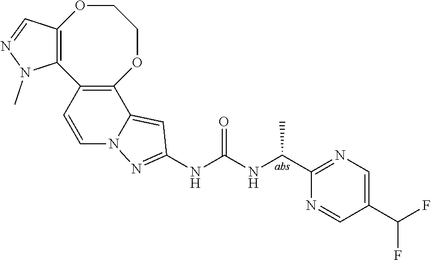 [SMILES: C[C@@H](NC(=O)Nc1cc2c3c(ccn2n1)-c1c(cnn1C)OCCO3)c1ncc(C(F)F)cn1] | LCMS-1 | 471 | 0.45 |
| I-254 | 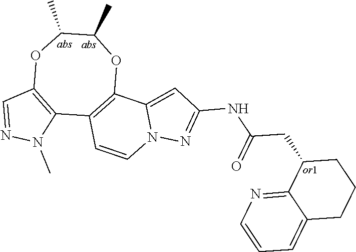 [SMILES: C[C@H]1Oc2cnn(C)c2-c2ccn3nc(NC(=O)C[C@@H]4CCCc5cccnc54)cc3c2O[C@@H]1C] | LCMS-1 | 473.2 | 0.437 |
| I-255 | 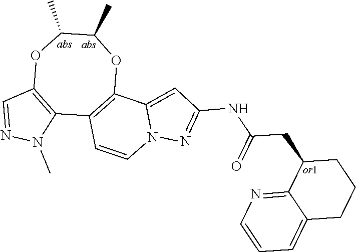 [SMILES: C[C@H]1Oc2cnn(C)c2-c2ccn3nc(NC(=O)C[C@H]4CCCc5cccnc54)cc3c2O[C@@H]1C] | LCMS-1 | 473.2 | 0.436 |
| I-256 | 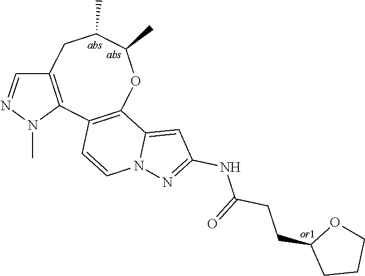 [SMILES: C[C@H]1Cc2cnn(C)c2-c2ccn3nc(NC(=O)CC[C@@H]4CCCO4)cc3c2O[C@@H]1C] | LCMS-1 | 426.2 | 0.493 |
| I-257 | 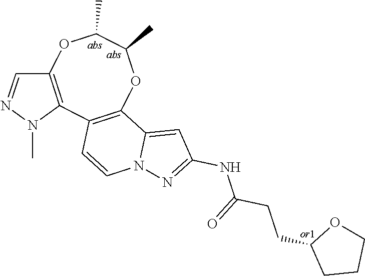 [SMILES: C[C@H]1Oc2cnn(C)c2-c2ccn3nc(NC(=O)CC[C@H]4CCCO4)cc3c2O[C@@H]1C] | LCMS-1 | 426.2 | 0.493 |
| I-258 | 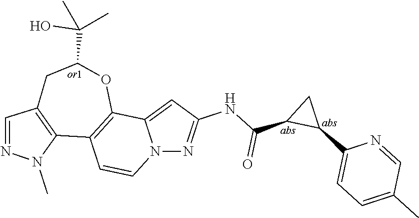 [SMILES: Cc1ccc([C@@H]2C[C@@H]2C(=O)Nc2cc3c4c(ccn3n2)-c2c(cnn2C)C[C@H](C(C)(C)O)O4)nc1] | LCMS-1 | 473.3 | 0.406 |
| I-259 | 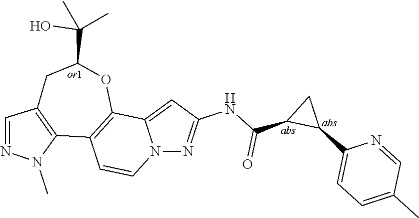 [SMILES: Cc1ccc([C@@H]2C[C@@H]2C(=O)Nc2cc3c4c(ccn3n2)-c2c(cnn2C)C[C@@H](C(C)(C)O)O4)nc1] | LCMS-1 | 473.3 | 0.406 |
| I-260 | 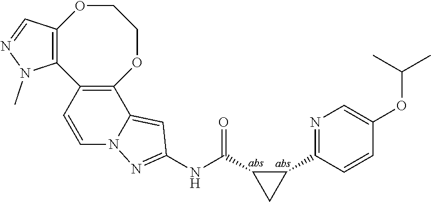 [SMILES: CC(C)Oc1ccc([C@@H]2C[C@@H]2C(=O)Nc2cc3c4c(ccn3n2)-c2c(cnn2C)OCCO4)nc1] | LCMS-1 | 475.2 | 0.41 |
| I-261 | 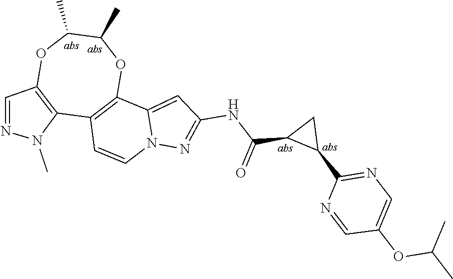 [SMILES: CC(C)Oc1cnc([C@@H]2C[C@@H]2C(=O)Nc2cc3c4c(ccn3n2)-c2c(cnn2C)O[C@H](C)[C@@H](C)O4)nc1] | LCMS-1 | 504.2 | 0.483 |
| I-262 | 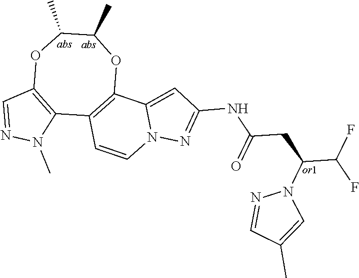 [SMILES: Cc1cnn([C@@H](CC(=O)Nc2cc3c4c(ccn3n2)-c2c(cnn2C)O[C@H](C)[C@@H](C)O4)C(F)F)c1] | LCMS-1 | 486.2 | 0.504 |
| I-263 | 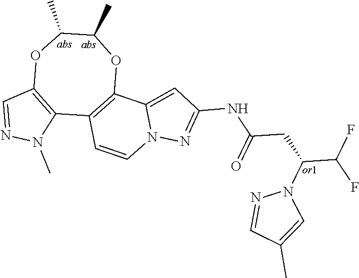 [SMILES: Cc1cnn([C@H](CC(=O)Nc2cc3c4c(ccn3n2)-c2c(cnn2C)O[C@H](C)[C@@H](C)O4)C(F)F)c1] | LCMS-1 | 486.2 | 0.51 |
| I-264 | 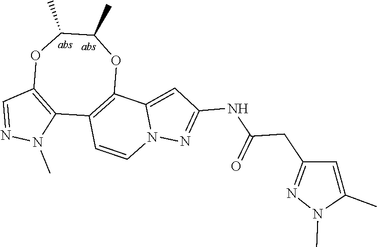 [SMILES: Cc1cc(CC(=O)Nc2cc3c4c(ccn3n2)-c2c(cnn2C)O[C@H](C)[C@@H](C)O4)nn1C] | LCMS-1 | 436.3 | 0.489 |
| I-265 | 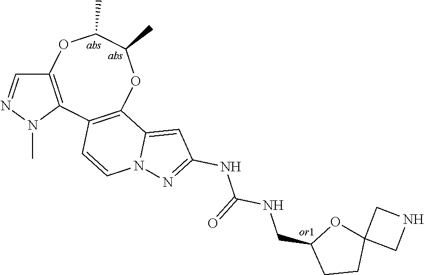 [SMILES: C[C@H]1Oc2cnn(C)c2-c2ccn3nc(NC(=O)NC[C@@H]4CCC5(CNC5)O4)cc3c2O[C@@H]1C] | LCMS-1 | 468.1 | 0.408 |
| I-266 | 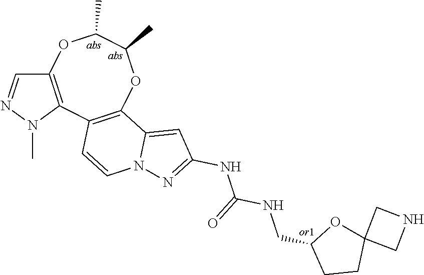 [SMILES: C[C@H]1Oc2cnn(C)c2-c2ccn3nc(NC(=O)NC[C@H]4CCC5(CNC5)O4)cc3c2O[C@@H]1C] | LCMS-1 | 468.1 | 0.404 |
| I-267 | 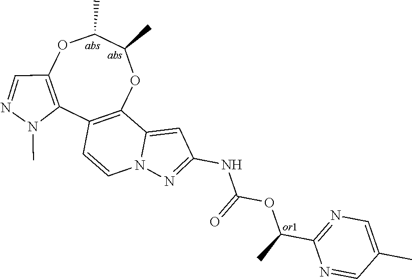 [SMILES: Cc1cnc([C@@H](C)OC(=O)Nc2cc3c4c(ccn3n2)-c2c(cnn2C)O[C@H](C)[C@@H](C)O4)nc1] | LCMS-1 | 464.2 | 0.489 |
| I-268 | 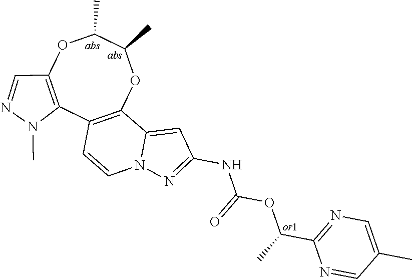 [SMILES: Cc1cnc([C@H](C)OC(=O)Nc2cc3c4c(ccn3n2)-c2c(cnn2C)O[C@H](C)[C@@H](C)O4)nc1] | LCMS-1 | 464.2 | 0.492 |
| I-269 | 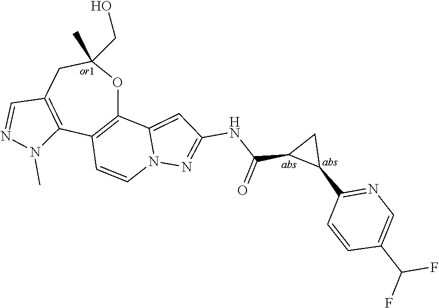 [SMILES: Cn1ncc2c1-c1ccn3nc(NC(=O)[C@H]4C[C@H]4c4ccc(C(F)F)cn4)cc3c1O[C@@](C)(CO)C2] | LCMS-1 | 495.3 | 0.42 |
| I-270 | 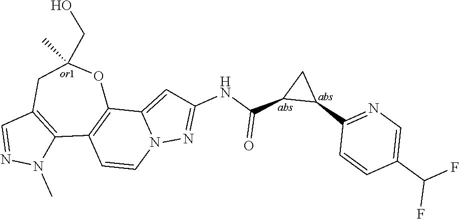 [SMILES: Cn1ncc2c1-c1ccn3nc(NC(=O)[C@H]4C[C@H]4c4ccc(C(F)F)cn4)cc3c1O[C@](C)(CO)C2] | LCMS-1 | 495.3 | 0.428 |
| I-271 | 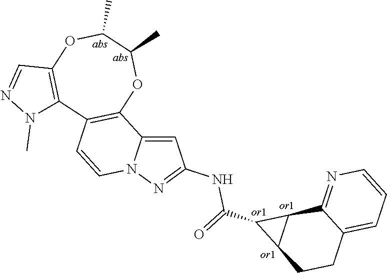 [SMILES: C[C@H]1Oc2cnn(C)c2-c2ccn3nc(NC(=O)[C@H]4[C@@H]5CCc6cccnc6[C@@H]54)cc3c2O[C@@H]1C] | LCMS-1 | 471.2 | 0.429 |
| I-272 | 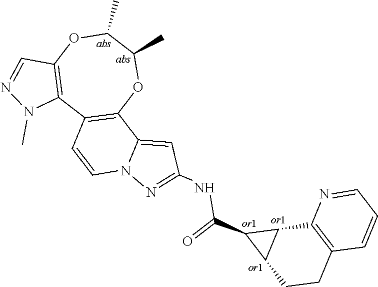 [SMILES: C[C@H]1Oc2cnn(C)c2-c2ccn3nc(NC(=O)[C@@H]4[C@H]5CCc6cccnc6[C@H]54)cc3c2O[C@@H]1C] | LCMS-1 | 471.2 | 0.423 |
| I-273 | 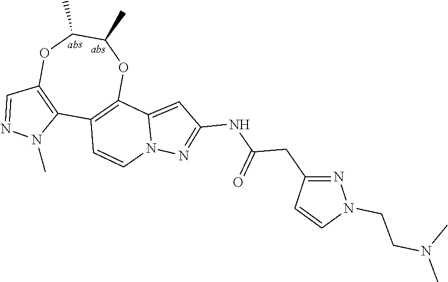 [SMILES: C[C@H]1Oc2cnn(C)c2-c2ccn3nc(NC(=O)Cc4ccn(CCN(C)C)n4)cc3c2O[C@@H]1C] | LCMS-1 | 479.2 | 0.434 |
| I-274 | 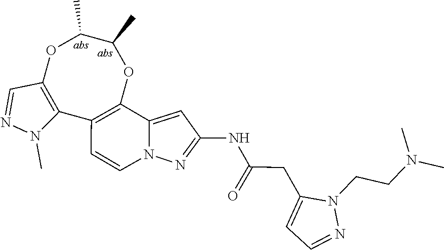 [SMILES: C[C@H]1Oc2cnn(C)c2-c2ccn3nc(NC(=O)Cc4ccnn4CCN(C)C)cc3c2O[C@@H]1C] | LCMS-1 | 479.2 | 0.441 |
| I-275 | 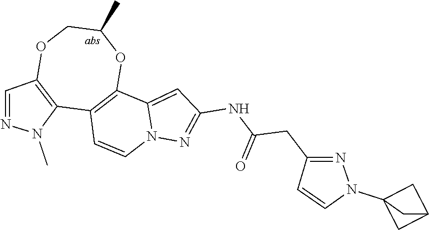 [SMILES: C[C@@H]1COc2cnn(C)c2-c2ccn3nc(NC(=O)Cc4ccn(C56CC(C5)C6)n4)cc3c2O1] | LCMS-1 | 460.1 | 0.531 |
| I-276 | 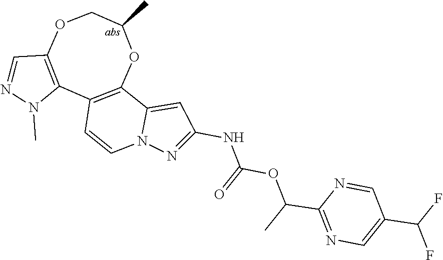 [SMILES: CC(OC(=O)Nc1cc2c3c(ccn2n1)-c1c(cnn1C)OC[C@@H](C)O3)c1ncc(C(F)F)cn1] | LCMS-1 | 486.3 | 0.464 |
| I-277 | 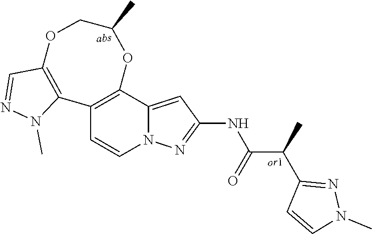 [SMILES: C[C@@H]1COc2cnn(C)c2-c2ccn3nc(NC(=O)[C@@H](C)c4ccn(C)n4)cc3c2O1] | LCMS-1 | 422.1 | 0.494 |
| I-278 | 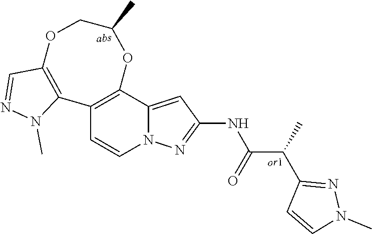 [SMILES: C[C@@H]1COc2cnn(C)c2-c2ccn3nc(NC(=O)[C@H](C)c4ccn(C)n4)cc3c2O1] | LCMS-1 | 422.1 | 0.496 |
| I-279 | 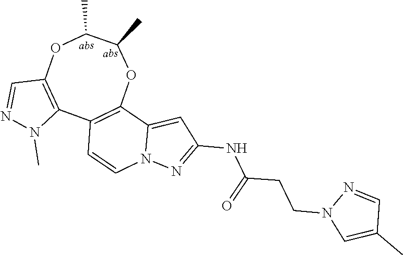 [SMILES: Cc1cnn(CCC(=O)Nc2cc3c4c(ccn3n2)-c2c(cnn2C)O[C@H](C)[C@@H](C)O4)c1] | LCMS-1 | 436.3 | 0.475 |
| I-280 | 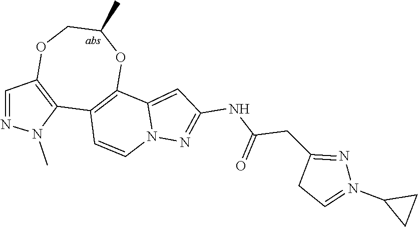 | LCMS-1 | 434.2 | 0.473 |
| I-281 | 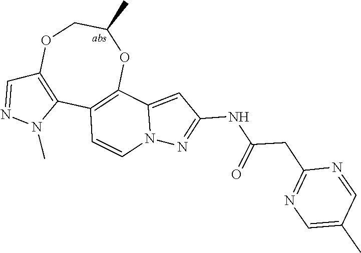 [SMILES: Cc1cnc(CC(=O)Nc2cc3c4c(ccn3n2)-c2c(cnn2C)OC[C@@H](C)O4)nc1] | LCMS-1 | 420.2 | 0.44 |
| I-282 | 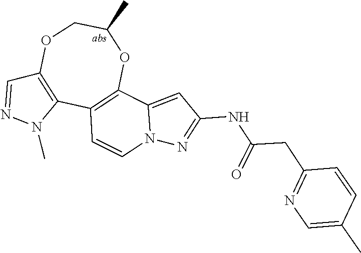 [SMILES: Cc1ccc(CC(=O)Nc2cc3c4c(ccn3n2)-c2c(cnn2C)OC[C@@H](C)O4)nc1] | LCMS-1 | 419.3 | 0.406 |
| I-283 | 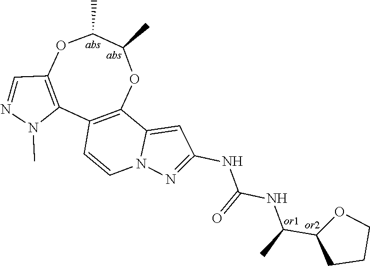 [SMILES: C[C@@H](NC(=O)Nc1cc2c3c(ccn2n1)-c1c(cnn1C)O[C@H](C)[C@@H](C)O3)[C@@H]1CCCO1] | LCMS-1 | 441.2 | 0.484 |
| I-284 | 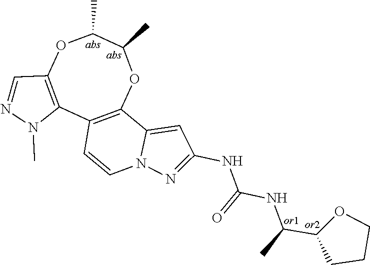 [SMILES: C[C@@H](NC(=O)Nc1cc2c3c(ccn2n1)-c1c(cnn1C)O[C@H](C)[C@@H](C)O3)[C@H]1CCCO1] | LCMS-1 | 441.2 | 0.479 |
| I-285 | 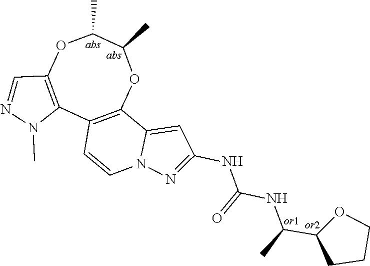 [SMILES: C[C@@H](NC(=O)Nc1cc2c3c(ccn2n1)-c1c(cnn1C)O[C@H](C)[C@@H](C)O3)[C@@H]1CCCO1] | LCMS-1 | 441.3 | 0.475 |
| I-286 | 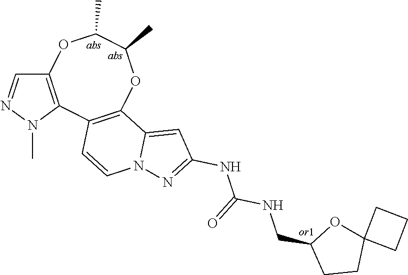 [SMILES: C[C@H]1Oc2cnn(C)c2-c2ccn3nc(NC(=O)NC[C@@H]4CCC5(CCC5)O4)cc3c2O[C@@H]1C] | LCMS-1 | 467.3 | 0.511 |
| I-287 | 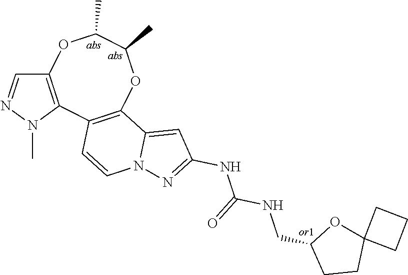 [SMILES: C[C@H]1Oc2cnn(C)c2-c2ccn3nc(NC(=O)NC[C@H]4CCC5(CCC5)O4)cc3c2O[C@@H]1C] | LCMS-1 | 467.3 | 0.519 |
| I-288 | 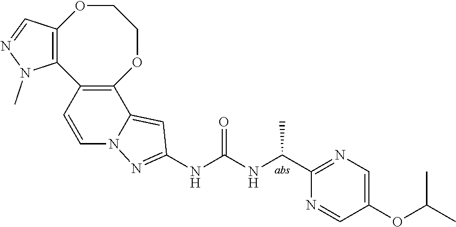 [SMILES: CC(C)Oc1cnc([C@@H](C)NC(=O)Nc2cc3c4c(ccn3n2)-c2c(cnn2C)OCCO4)nc1] | LCMS-1 | 479.3 | 0.455 |
| I-289 | 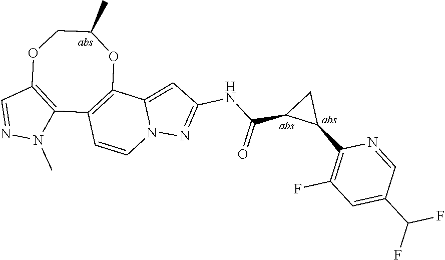 [SMILES: C[C@@H]1COc2cnn(C)c2-c2ccn3nc(NC(=O)[C@H]4C[C@H]4c4ncc(C(F)F)cc4F)cc3c2O1] | LCMS-1 | 499.1 | 0.528 |
| I-290 | 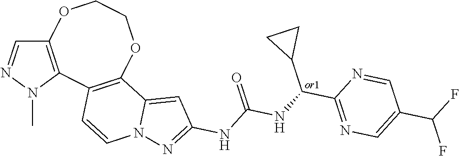 [SMILES: Cn1ncc2c1-c1ccn3nc(NC(=O)N[C@@H](c4ncc(C(F)F)cn4)C4CC4)cc3c1OCCO2] | LCMS-1 | 497.2 | 0.503 |
| I-291 | 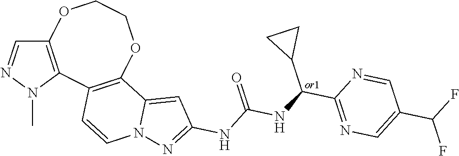 [SMILES: Cn1ncc2c1-c1ccn3nc(NC(=O)N[C@H](c4ncc(C(F)F)cn4)C4CC4)cc3c1OCCO2] | LCMS-1 | 497.2 | 0.507 |
| I-292 | 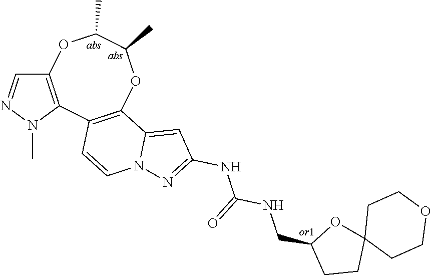 [SMILES: C[C@H]1Oc2cnn(C)c2-c2ccn3nc(NC(=O)NC[C@@H]4CCC5(CCOCC5)O4)cc3c2O[C@@H]1C] | LCMS-1 | 497.2 | 0.468 |
| I-293 | 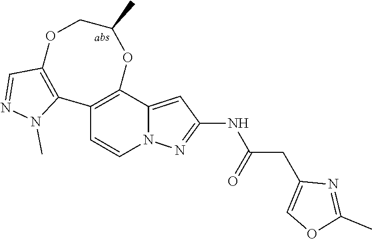 [SMILES: Cc1nc(CC(=O)Nc2cc3c4c(ccn3n2)-c2c(cnn2C)OC[C@@H](C)O4)co1] | LCMS-1 | 409.2 | 0.445 |
| I-294 | 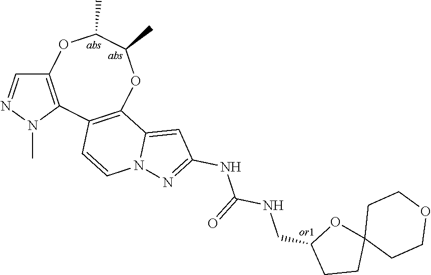 [SMILES: C[C@H]1Oc2cnn(C)c2-c2ccn3nc(NC(=O)NC[C@H]4CCC5(CCOCC5)O4)cc3c2O[C@@H]1C] | LCMS-1 | 497.2 | 0.467 |
| I-295 | 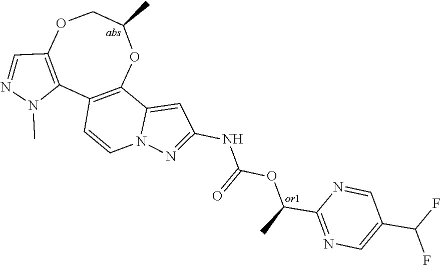 [SMILES: C[C@@H]1COc2cnn(C)c2-c2ccn3nc(NC(=O)O[C@H](C)c4ncc(C(F)F)cn4)cc3c2O1] | LCMS-1 | 486.2 | 0.528 |
| I-296 | 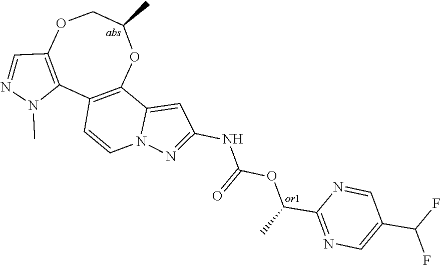 [SMILES: C[C@@H]1COc2cnn(C)c2-c2ccn3nc(NC(=O)O[C@@H](C)c4ncc(C(F)F)cn4)cc3c2O1] | LCMS-1 | 486.1 | 0.525 |
| I-297 | 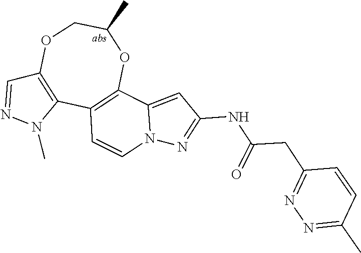 [SMILES: Cc1ccc(CC(=O)Nc2cc3c4c(ccn3n2)-c2c(cnn2C)OC[C@@H](C)O4)nn1] | LCMS-1 | 420.1 | 0.449 |
| I-298 | 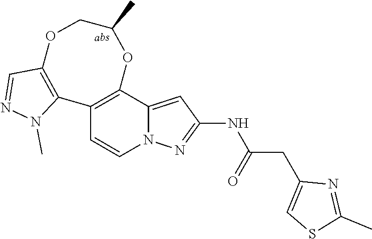 [SMILES: Cc1nc(CC(=O)Nc2cc3c4c(ccn3n2)-c2c(cnn2C)OC[C@@H](C)O4)cs1] | LCMS-1 | 425.1 | 0.446 |
| I-299 | 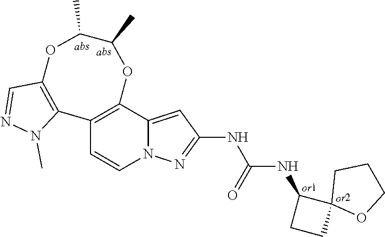 [SMILES: C[C@H]1Oc2cnn(C)c2-c2ccn3nc(NC(=O)N[C@@H]4CC[C@@]45CCCO5)cc3c2O[C@@H]1C] | LCMS-1 | 453.1 | 0.538 |
| I-300 | 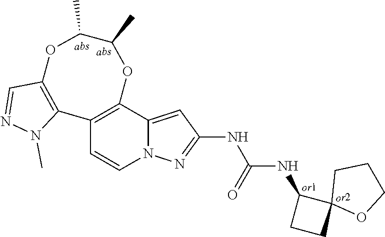 [SMILES: C[C@H]1Oc2cnn(C)c2-c2ccn3nc(NC(=O)N[C@@H]4CC[C@]45CCCO5)cc3c2O[C@@H]1C] | LCMS-1 | 453.2 | 0.533 |
| I-301 | 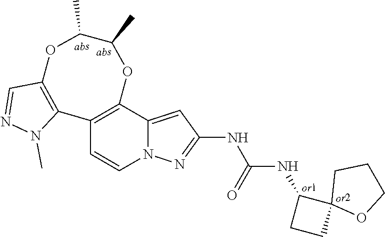 [SMILES: C[C@H]1Oc2cnn(C)c2-c2ccn3nc(NC(=O)N[C@H]4CC[C@@]45CCCO5)cc3c2O[C@@H]1C] | LCMS-1 | 453.1 | 0.512 |
| I-302 | 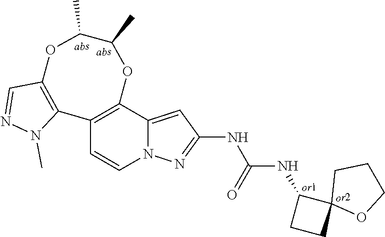 [SMILES: C[C@H]1Oc2cnn(C)c2-c2ccn3nc(NC(=O)N[C@H]4CC[C@]45CCCO5)cc3c2O[C@@H]1C] | LCMS-1 | 453.1 | 0.525 |
| I-303 | 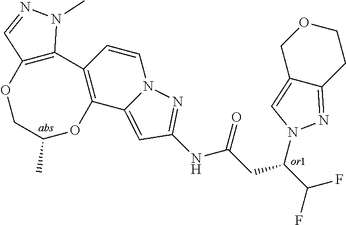 [SMILES: C[C@@H]1COc2cnn(C)c2-c2ccn3nc(NC(=O)C[C@@H](C(F)F)n4cc5c(n4)CCOC5)cc3c2O1] | LCMS-1 | 514.3 | 0.448 |
| I-304 | 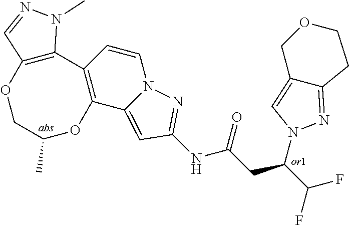 [SMILES: C[C@@H]1COc2cnn(C)c2-c2ccn3nc(NC(=O)C[C@H](C(F)F)n4cc5c(n4)CCOC5)cc3c2O1] | LCMS-1 | 514.3 | 0.447 |
| I-305 | 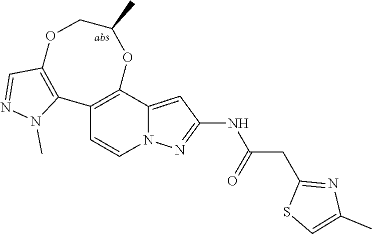 [SMILES: Cc1csc(CC(=O)Nc2cc3c4c(ccn3n2)-c2c(cnn2C)OC[C@@H](C)O4)n1] | LCMS-1 | 425.3 | 0.449 |
| I-306 | 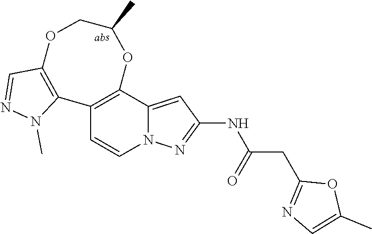 [SMILES: Cc1cnc(CC(=O)Nc2cc3c4c(ccn3n2)-c2c(cnn2C)OC[C@@H](C)O4)o1] | LCMS-1 | 409.2 | 0.45 |
| I-307 | 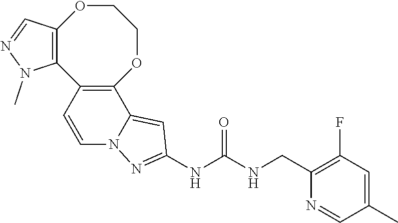 [SMILES: Cc1cnc(CNC(=O)Nc2cc3c4c(ccn3n2)-c2c(cnn2C)OCCO4)c(F)c1] | LCMS-2 | 438.2 | 0.531 |
| I-308 | 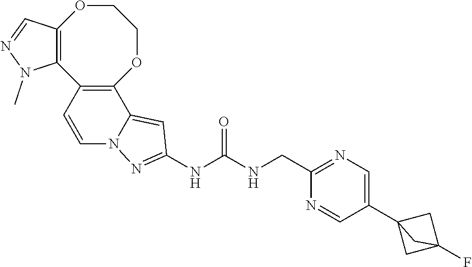 [SMILES: Cn1ncc2c1-c1ccn3nc(NC(=O)NCc4ncc(C56CC(F)(C5)C6)cn4)cc3c1OCCO2] | LCMS-1 | 491.2 | 0.473 |
| I-309 | 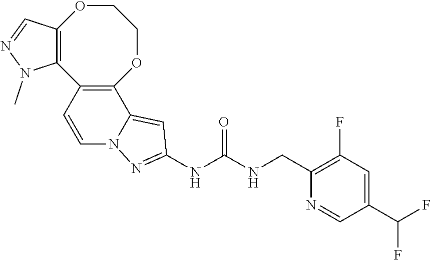 [SMILES: Cn1ncc2c1-c1ccn3nc(NC(=O)NCc4ncc(C(F)F)cc4F)cc3c1OCCO2] | LCMS-1 | 474.1 | 0.461 |
| I-310 | 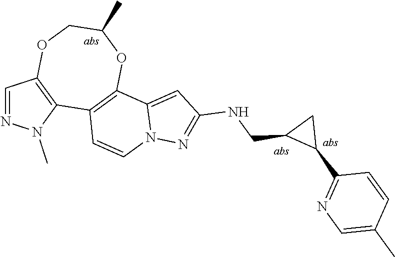 [SMILES: Cc1ccc([C@@H]2C[C@@H]2CNc2cc3c4c(ccn3n2)-c2c(cnn2C)OC[C@@H](C)O4)nc1] | LCMS-1 | 431.2 | 0.4 |
| I-311 | 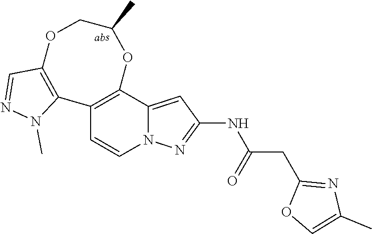 [SMILES: Cc1coc(CC(=O)Nc2cc3c4c(ccn3n2)-c2c(cnn2C)OC[C@@H](C)O4)n1] | LCMS-1 | 409 | 0.488 |
| I-312 | 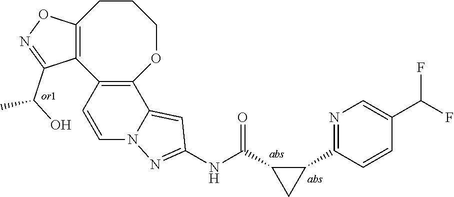 [SMILES: C[C@@H](O)c1noc2c1-c1ccn3nc(NC(=O)[C@H]4C[C@H]4c4ccc(C(F)F)cn4)cc3c1OCCC2] | LCMS-1 | 496.1 | 0.467 |
| I-313 | 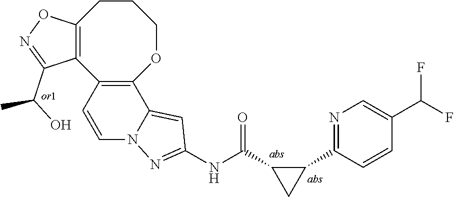 [SMILES: C[C@H](O)c1noc2c1-c1ccn3nc(NC(=O)[C@H]4C[C@H]4c4ccc(C(F)F)cn4)cc3c1OCCC2] | LCMS-1 | 496.1 | 0.47 |
| I-314 | 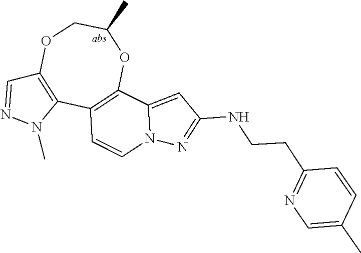 [SMILES: Cc1ccc(CCNc2cc3c4c(ccn3n2)-c2c(cnn2C)OC[C@@H](C)O4)nc1] | LCMS-1 | 405.2 | 0.409 |
| I-315 | 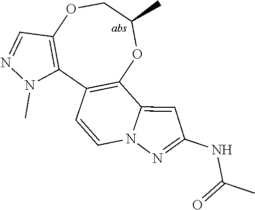 [SMILES: CC(=O)Nc1cc2c3c(ccn2n1)-c1c(cnn1C)OC[C@@H](C)O3] | LCMS-1 | 328.2 | 0.415 |
| I-316 | 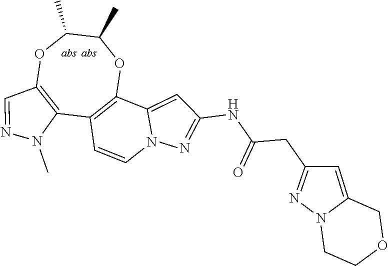 [SMILES: C[C@H]1Oc2cnn(C)c2-c2ccn3nc(NC(=O)Cc4cc5n(n4)CCOC5)cc3c2O[C@@H]1C] | LCMS-1 | 464.2 | 0.45 |
| I-317 | 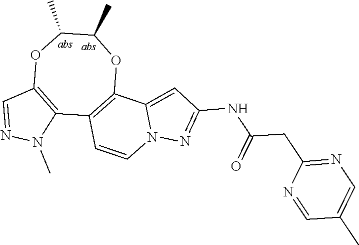 [SMILES: Cc1cnc(CC(=O)Nc2cc3c4c(ccn3n2)-c2c(cnn2C)O[C@H](C)[C@@H](C)O4)nc1] | LCMS-1 | 434.2 | 0.457 |
| I-318 | 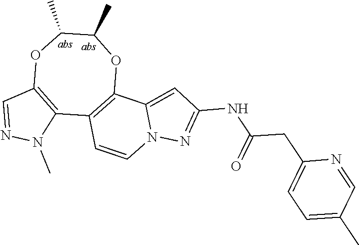 [SMILES: Cc1ccc(CC(=O)Nc2cc3c4c(ccn3n2)-c2c(cnn2C)O[C@H](C)[C@@H](C)O4)nc1] | LCMS-1 | 433.2 | 0.415 |
| I-319 | 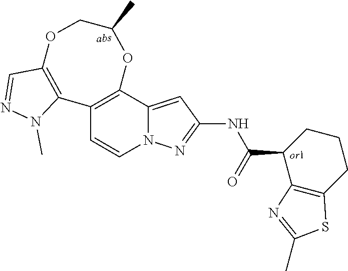 [SMILES: Cc1nc2c(s1)CCC[C@@H]2C(=O)Nc1cc2c3c(ccn2n1)-c1c(cnn1C)OC[C@@H](C)O3] | LCMS-1 | 465.3 | 0.514 |
| I-320 | 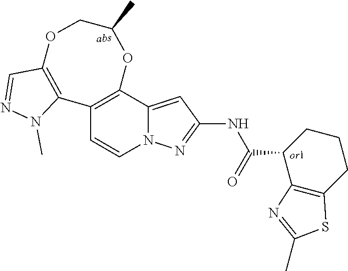 [SMILES: Cc1nc2c(s1)CCC[C@H]2C(=O)Nc1cc2c3c(ccn2n1)-c1c(cnn1C)OC[C@@H](C)O3] | LCMS-1 | 465.3 | 0.51 |
| I-321 | 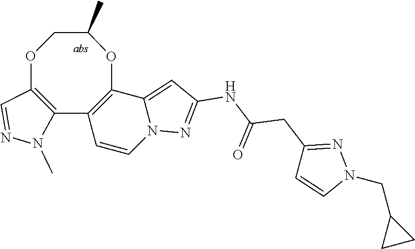 [SMILES: C[C@@H]1COc2cnn(C)c2-c2ccn3nc(NC(=O)Cc4ccn(CC5CC5)n4)cc3c2O1] | LCMS-1 | 448.2 | 0.486 |
| I-322 | 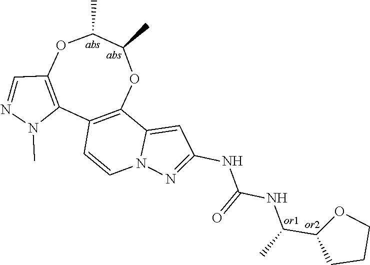 [SMILES: C[C@H](NC(=O)Nc1cc2c3c(ccn2n1)-c1c(cnn1C)O[C@H](C)[C@@H](C)O3)[C@H]1CCCO1] | LCMS-1 | 441.2 | 0.486 |
| I-323 | 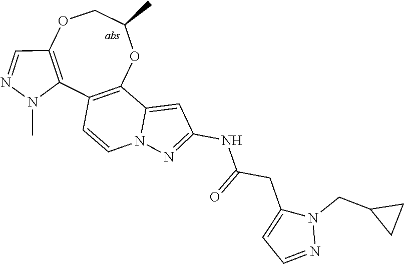 [SMILES: C[C@@H]1COc2cnn(C)c2-c2ccn3nc(NC(=O)Cc4ccnn4CC4CC4)cc3c2O1] | LCMS-1 | 448.2 | 0.459 |
| I-324 | 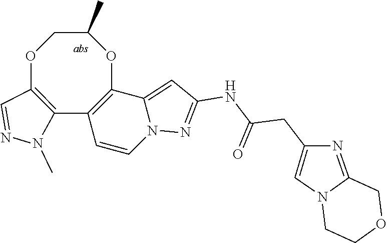 [SMILES: C[C@@H]1COc2cnn(C)c2-c2ccn3nc(NC(=O)Cc4cn5c(n4)COCC5)cc3c2O1] | LCMS-1 | 450.2 | 0.373 |
| I-325 | 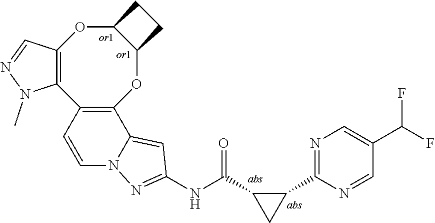 [SMILES: Cn1ncc2c1-c1ccn3nc(NC(=O)[C@H]4C[C@H]4c4ncc(C(F)F)cn4)cc3c1O[C@@H]1CC[C@@H]1O2] | LCMS-1 | 494.2 | 0.47 |
| I-326 | 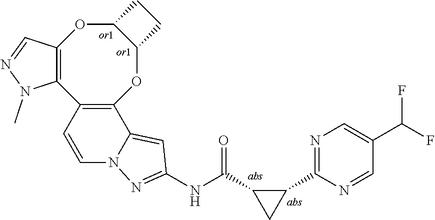 [SMILES: Cn1ncc2c1-c1ccn3nc(NC(=O)[C@H]4C[C@H]4c4ncc(C(F)F)cn4)cc3c1O[C@H]1CC[C@H]1O2] | LCMS-1 | 494.2 | 0.473 |
| I-327 | 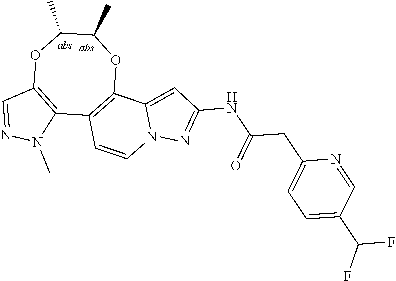 [SMILES: C[C@H]1Oc2cnn(C)c2-c2ccn3nc(NC(=O)Cc4ccc(C(F)F)cn4)cc3c2O[C@@H]1C] | LCMS-1 | 469.2 | 0.509 |
| I-328 | 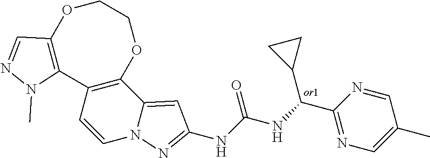 [SMILES: Cc1cnc([C@H](NC(=O)Nc2cc3c4c(ccn3n2)-c2c(cnn2C)OCCO4)C2CC2)nc1] | LCMS-1 | 461.2 | 0.458 |
| I-329 | 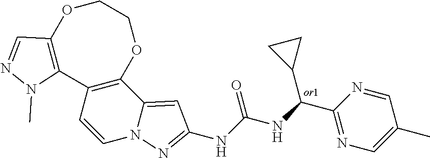 [SMILES: Cc1cnc([C@@H](NC(=O)Nc2cc3c4c(ccn3n2)-c2c(cnn2C)OCCO4)C2CC2)nc1] | LCMS-1 | 461.2 | 0.457 |
| I-330 | 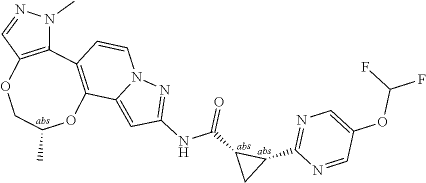 [SMILES: C[C@@H]1COc2cnn(C)c2-c2ccn3nc(NC(=O)[C@H]4C[C@H]4c4ncc(OC(F)F)cn4)cc3c2O1] | LCMS-1 | 498.2 | 0.447 |
| I-331 | 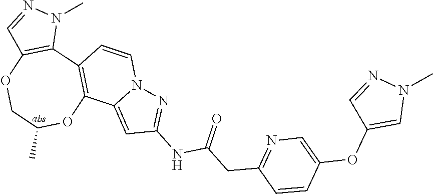 [SMILES: C[C@@H]1COc2cnn(C)c2-c2ccn3nc(NC(=O)Cc4ccc(Oc5cnn(C)c5)cn4)cc3c2O1] | LCMS-1 | 501.2 | 0.435 |
| I-332 | 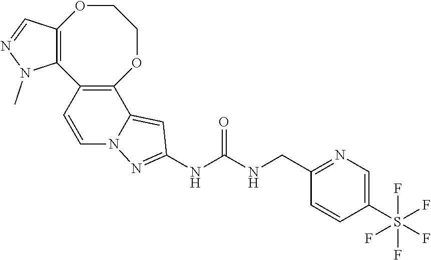 [SMILES: Cn1ncc2c1-c1ccn3nc(NC(=O)NCc4ccc(S(F)(F)(F)(F)F)cn4)cc3c1OCCO2] | LCMS-1 | 532.1 | 0.501 |
| I-333 | 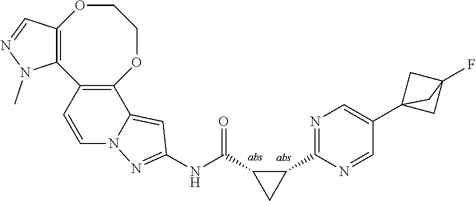 [SMILES: Cn1ncc2c1-c1ccn3nc(NC(=O)[C@H]4C[C@H]4c4ncc(C56CC(F)(C5)C6)cn4)cc3c1OCCO2] | LCMS-1 | 502.3 | 0.478 |
| I-334 | 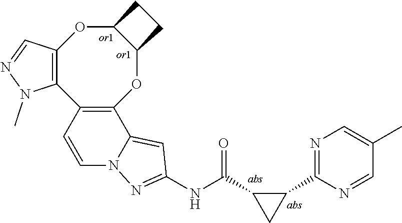 [SMILES: Cc1cnc([C@@H]2C[C@@H]2C(=O)Nc2cc3c4c(ccn3n2)-c2c(cnn2C)O[C@H]2CC[C@H]2O4)nc1] | LCMS-1 | 458.3 | 0.444 |
| I-335 | 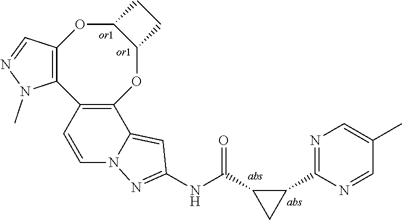 [SMILES: Cc1cnc([C@@H]2C[C@@H]2C(=O)Nc2cc3c4c(ccn3n2)-c2c(cnn2C)O[C@@H]2CC[C@@H]2O4)nc1] | LCMS-1 | 458.2 | 0.447 |
| I-336 | 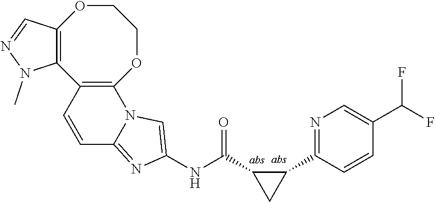 [SMILES: Cn1ncc2c1-c1ccc3nc(NC(=O)[C@H]4C[C@H]4c4ccc(C(F)F)cn4)cn3c1OCCO2] | LCMS-1 | 467.2 | 0.425 |
| I-337 | 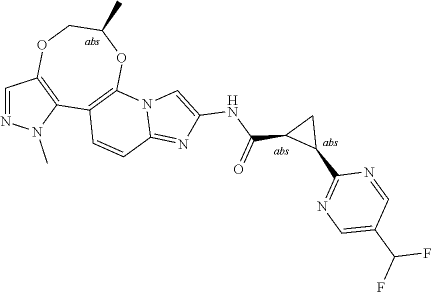 [SMILES: C[C@@H]1COc2cnn(C)c2-c2ccc3nc(NC(=O)[C@H]4C[C@H]4c4ncc(C(F)F)cn4)cn3c2O1] | LCMS-1 | 482.1 | 0.413 |
| I-338 | 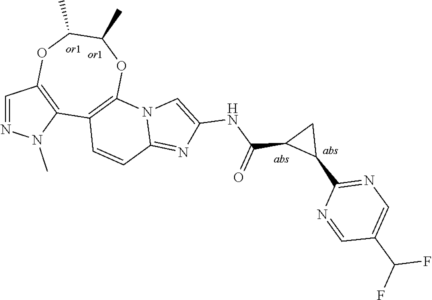 [SMILES: C[C@H]1Oc2cnn(C)c2-c2ccc3nc(NC(=O)[C@H]4C[C@H]4c4ncc(C(F)F)cn4)cn3c2O[C@@H]1C] | LCMS-1 | 496.2 | 0.432 |
| I-339 | 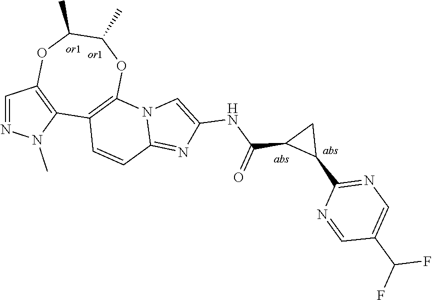 [SMILES: C[C@@H]1Oc2cnn(C)c2-c2ccc3nc(NC(=O)[C@H]4C[C@H]4c4ncc(C(F)F)cn4)cn3c2O[C@H]1C] | LCMS-1 | 496.3 | 0.427 |
| I-340 | 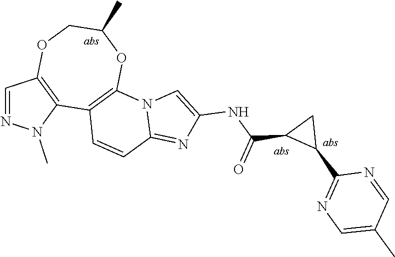 [SMILES: Cc1cnc([C@@H]2C[C@@H]2C(=O)Nc2cn3c4c(ccc3n2)-c2c(cnn2C)OC[C@@H](C)O4)nc1] | LCMS-1 | 446.2 | 0.396 |
| I-341 | 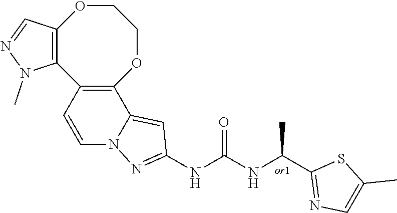 [SMILES: Cc1cnc([C@H](C)NC(=O)Nc2cc3c4c(ccn3n2)-c2c(cnn2C)OCCO4)s1] | LCMS-1 | 440.2 | 0.452 |
| I-432 | 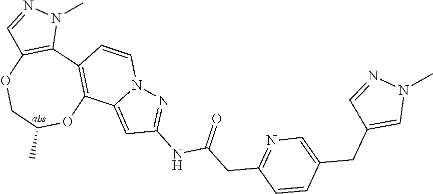 [SMILES: C[C@@H]1COc2cnn(C)c2-c2ccn3nc(NC(=O)Cc4ccc(Cc5cnn(C)c5)cn4)cc3c2O1] | LCMS-1 | 499.2 | 0.408 |
| I-343 | 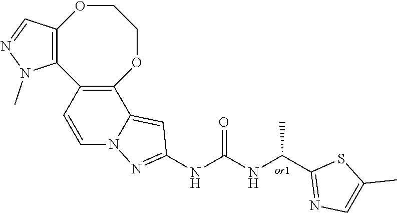 [SMILES: Cc1cnc([C@@H](C)NC(=O)Nc2cc3c4c(ccn3n2)-c2c(cnn2C)OCCO4)s1] | LCMS-1 | 440.1 | 0.457 |
| I-344 | 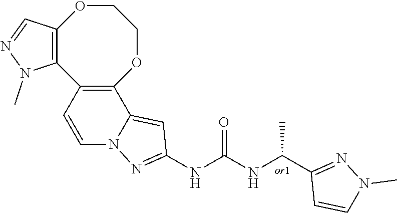 [SMILES: C[C@@H](NC(=O)Nc1cc2c3c(ccn2n1)-c1c(cnn1C)OCCO3)c1ccn(C)n1] | LCMS-1 | 423.2 | 0.436 |
| I-345 | 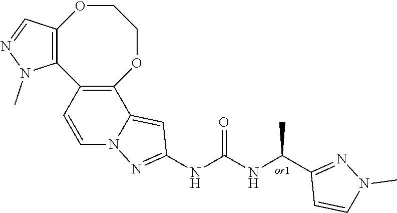 [SMILES: C[C@H](NC(=O)Nc1cc2c3c(ccn2n1)-c1c(cnn1C)OCCO3)c1ccn(C)n1] | LCMS-1 | 423.2 | 0.427 |
| I-346 | 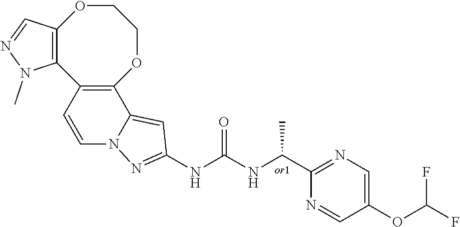 [SMILES: C[C@@H](NC(=O)Nc1cc2c3c(ccn2n1)-c1c(cnn1C)OCCO3)c1ncc(OC(F)F)cn1] | LCMS-1 | 487.2 | 0.444 |
| I-347 | 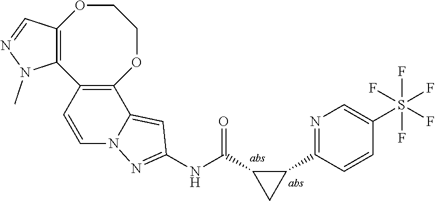 [SMILES: Cn1ncc2c1-c1ccn3nc(NC(=O)[C@H]4C[C@H]4c4ccc(S(F)(F)(F)(F)F)cn4)cc3c1OCCO2] | LCMS-1 | 543.1 | 0.511 |
| I-348 | 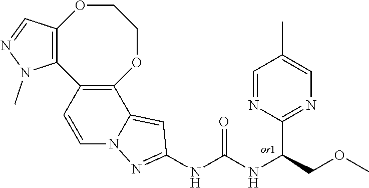 [SMILES: COC[C@@H](NC(=O)Nc1cc2c3c(ccn2n1)-c1c(cnn1C)OCCO3)c1ncc(C)cn1] | LCMS-1 | 465.2 | 0.431 |
| I-349 | 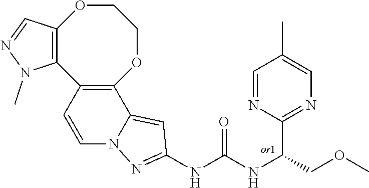 [SMILES: COC[C@H](NC(=O)Nc1cc2c3c(ccn2n1)-c1c(cnn1C)OCCO3)c1ncc(C)cn1] | LCMS-1 | 465.2 | 0.427 |
| I-350 | 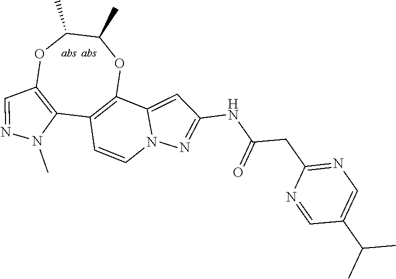 [SMILES: CC(C)c1cnc(CC(=O)Nc2cc3c4c(ccn3n2)-c2c(cnn2C)O[C@H](C)[C@@H](C)O4)nc1] | LCMS-1 | 462.2 | 0.492 |
| I-351 | 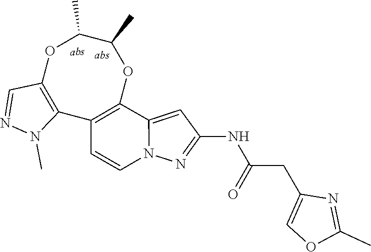 [SMILES: Cc1nc(CC(=O)Nc2cc3c4c(ccn3n2)-c2c(cnn2C)O[C@H](C)[C@@H](C)O4)co1] | LCMS-1 | 423.2 | 0.463 |
| I-352 | 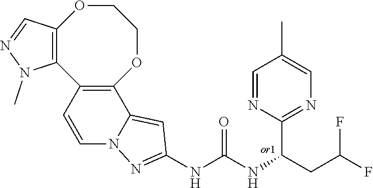 [SMILES: Cc1cnc([C@H](CC(F)F)NC(=O)Nc2cc3c4c(ccn3n2)-c2c(cnn2C)OCCO4)nc1] | LCMS-1 | 485.3 | 0.435 |
| I-353 | 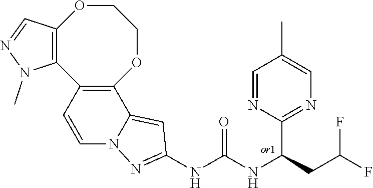 [SMILES: Cc1cnc([C@@H](CC(F)F)NC(=O)Nc2cc3c4c(ccn3n2)-c2c(cnn2C)OCCO4)nc1] | LCMS-1 | 485.2 | 0.434 |
| I-354 | 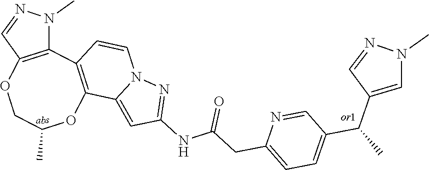 [SMILES: C[C@H](c1ccc(CC(=O)Nc2cc3c4c(ccn3n2)-c2c(cnn2C)OC[C@@H](C)O4)nc1)c1cnn(C)c1] | LCMS-1 | 513.3 | 0.416 |
| I-355 | 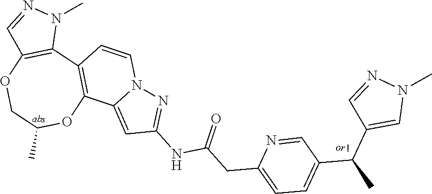 [SMILES: C[C@@H](c1ccc(CC(=O)Nc2cc3c4c(ccn3n2)-c2c(cnn2C)OC[C@@H](C)O4)nc1)c1cnn(C)c1] | LCMS-1 | 513.4 | 0.419 |
| I-356 | 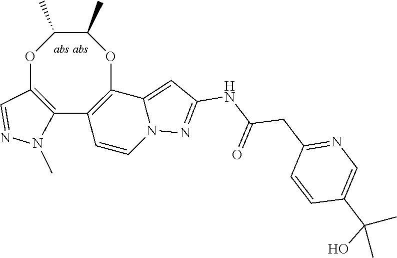 [SMILES: C[C@H]1Oc2cnn(C)c2-c2ccn3nc(NC(=O)Cc4ccc(C(C)(C)O)cn4)cc3c2O[C@@H]1C] | LCMS-1 | 477.3 | 0.436 |
| I-357 | 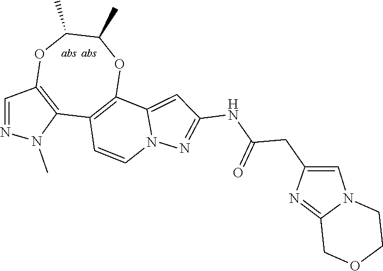 [SMILES: C[C@H]1Oc2cnn(C)c2-c2ccn3nc(NC(=O)Cc4cn5c(n4)COCC5)cc3c2O[C@@H]1C] | LCMS-1 | 464.2 | 0.382 |
| I-358 | 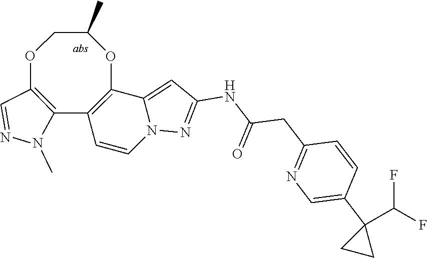 [SMILES: C[C@@H]1COc2cnn(C)c2-c2ccn3nc(NC(=O)Cc4ccc(C5(C(F)F)CC5)cn4)cc3c2O1] | LCMS-1 | 495.2 | 0.458 |
| I-359 | 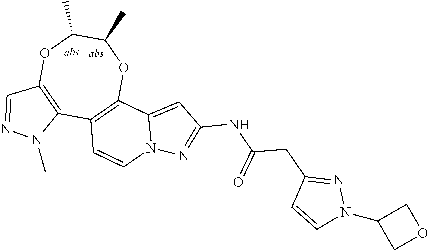 [SMILES: C[C@H]1Oc2cnn(C)c2-c2ccn3nc(NC(=O)Cc4ccn(C5COC5)n4)cc3c2O[C@@H]1C] | LCMS-1 | 464.2 | 0.45 |
| I-360 | 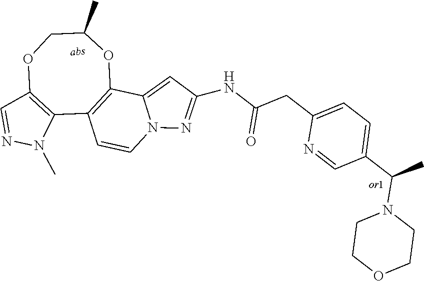 [SMILES: C[C@@H]1COc2cnn(C)c2-c2ccn3nc(NC(=O)Cc4ccc([C@@H](C)N5CCOCC5)cn4)cc3c2O1] | LCMS-1 | 518.3 | 0.395 |
| I-361 | 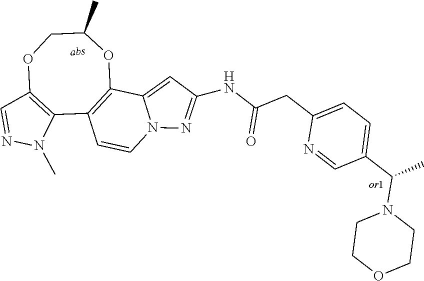 [SMILES: C[C@@H]1COc2cnn(C)c2-c2ccn3nc(NC(=O)Cc4ccc([C@H](C)N5CCOCC5)cn4)cc3c2O1] | LCMS-1 | 518.3 | 0.396 |
| I-362 | 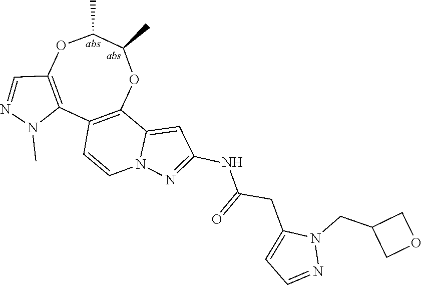 [SMILES: C[C@H]1Oc2cnn(C)c2-c2ccn3nc(NC(=O)Cc4ccnn4CC4COC4)cc3c2O[C@@H]1C] | LCMS-1 | 478.3 | 0.425 |
| I-363 | 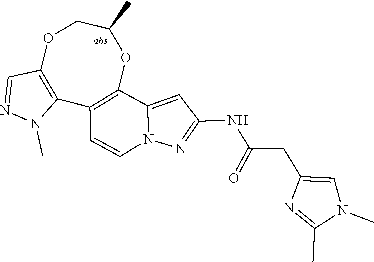 [SMILES: Cc1nc(CC(=O)Nc2cc3c4c(ccn3n2)-c2c(cnn2C)OC[C@@H](C)O4)cn1C] | LCMS-1 | 422.2 | 0.395 |
| I-364 | 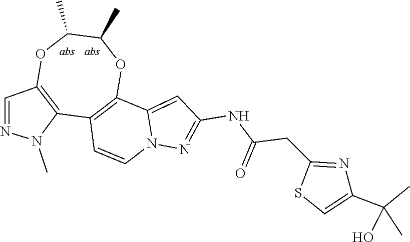 [SMILES: C[C@H]1Oc2cnn(C)c2-c2ccn3nc(NC(=O)Cc4nc(C(C)(C)O)cs4)cc3c2O[C@@H]1C] | LCMS-1 | 483 | 0.507 |
| I-365 | 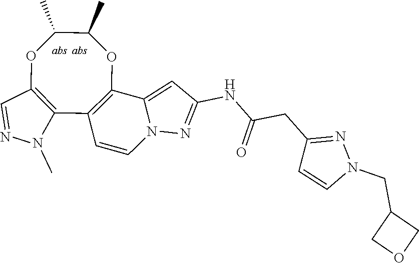 [SMILES: C[C@H]1Oc2cnn(C)c2-c2ccn3nc(NC(=O)Cc4ccn(CC5COC5)n4)cc3c2O[C@@H]1C] | LCMS-1 | 478.3 | 0.436 |
| I-366 | 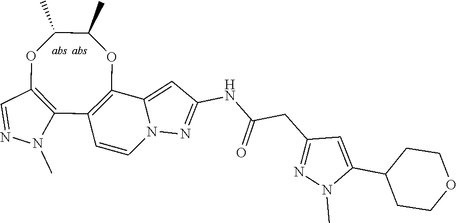 [SMILES: C[C@H]1Oc2cnn(C)c2-c2ccn3nc(NC(=O)Cc4cc(C5CCOCC5)n(C)n4)cc3c2O[C@@H]1C] | LCMS-1 | 506.3 | 0.471 |
| I-367 | 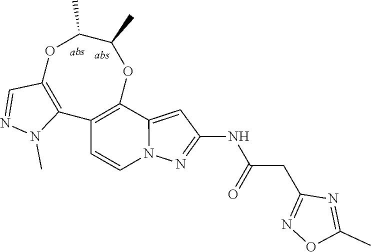 [SMILES: Cc1nc(CC(=O)Nc2cc3c4c(ccn3n2)-c2c(cnn2C)O[C@H](C)[C@@H](C)O4)no1] | LCMS-1 | 424.2 | 0.462 |
| I-368 | 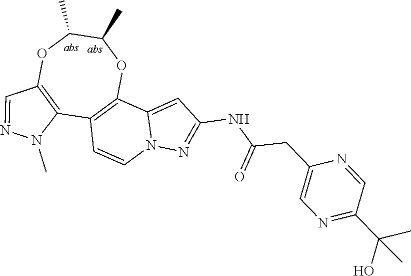 [SMILES: C[C@H]1Oc2cnn(C)c2-c2ccn3nc(NC(=O)Cc4cnc(C(C)(C)O)cn4)cc3c2O[C@@H]1C] | LCMS-1 | 478.3 | 0.453 |
| I-369 | 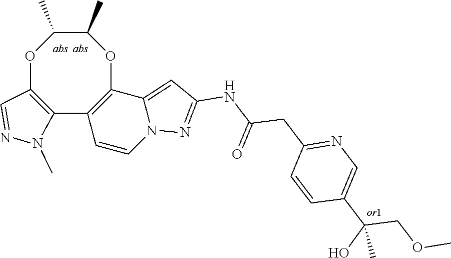 [SMILES: COC[C@@](C)(O)c1ccc(CC(=O)Nc2cc3c4c(ccn3n2)-c2c(cnn2C)O[C@H](C)[C@@H](C)O4)nc1] | LCMS-1 | 507.2 | 0.407 |
| I-370 | 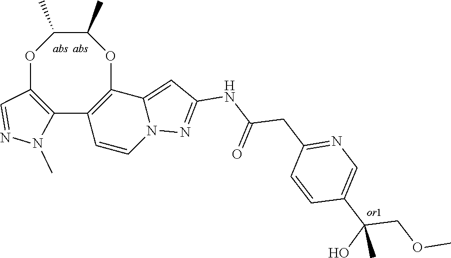 [SMILES: COC[C@](C)(O)c1ccc(CC(=O)Nc2cc3c4c(ccn3n2)-c2c(cnn2C)O[C@H](C)[C@@H](C)O4)nc1] | LCMS-1 | 507.2 | 0.405 |
| I-371 | 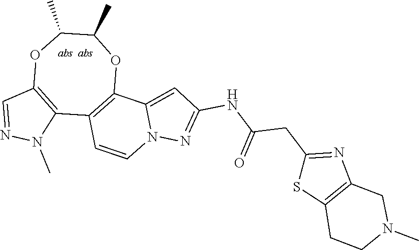 [SMILES: C[C@H]1Oc2cnn(C)c2-c2ccn3nc(NC(=O)Cc4nc5c(s4)CCN(C)C5)cc3c2O[C@@H]1C] | LCMS-1 | 494 | 0.408 |
| I-372 | 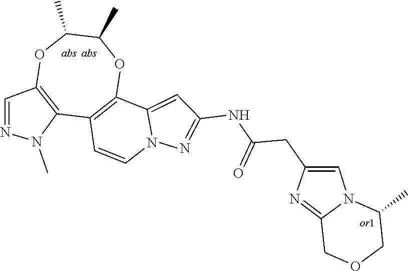 [SMILES: C[C@@H]1COCc2nc(CC(=O)Nc3cc4c5c(ccn4n3)-c3c(cnn3C)O[C@H](C)[C@@H](C)O5)cn21] | LCMS-1 | 478.1 | 0.405 |
| I-373 | 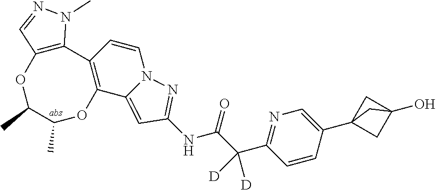 [SMILES: [2H]C([2H])(C(=O)Nc1cc2c3c(ccn2n1)-c1c(cnn1C)O[C@H](C)[C@@H](C)O3)c1ccc(C23CC(O)(C2)C3)cn1] | LCMS-2 | 503.2 | 0.799 |
| I-374 | 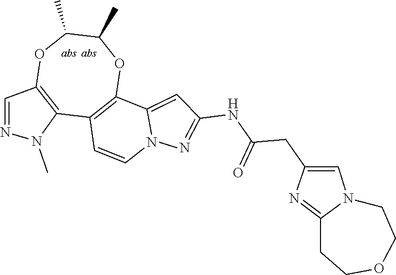 [SMILES: C[C@H]1Oc2cnn(C)c2-c2ccn3nc(NC(=O)Cc4cn5c(n4)CCOCC5)cc3c2O[C@@H]1C] | LCMS-1 | 478.3 | 0.387 |
| I-375 | 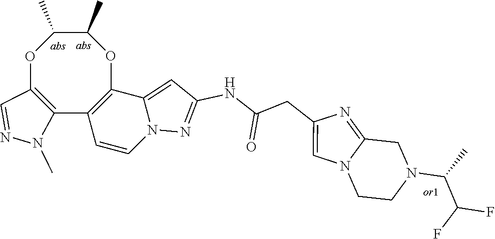 [SMILES: C[C@H](C(F)F)N1CCn2cc(CC(=O)Nc3cc4c5c(ccn4n3)-c3c(cnn3C)O[C@H](C)[C@@H](C)O5)nc2C1] | LCMS-1 | 541.3 | 0.415 |
| I-376 | 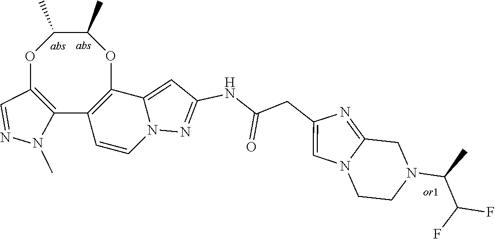 [SMILES: C[C@H]1Oc2cnn(C)c2-c2ccn3nc(NC(=O)Cc4cn5c(n4)CN([C@@H](C)C(F)F)CC5)cc3c2O[C@@H]1C] | LCMS-1 | 541.2 | 0.416 |
| I-377 | 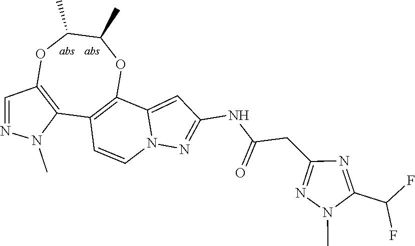 [SMILES: C[C@H]1Oc2cnn(C)c2-c2ccn3nc(NC(=O)Cc4nc(C(F)F)n(C)n4)cc3c2O[C@@H]1C] | LCMS-1 | 473.2 | 0.462 |
| I-378 | 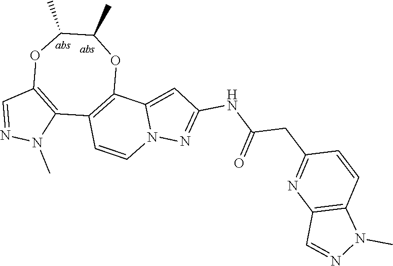 [SMILES: C[C@H]1Oc2cnn(C)c2-c2ccn3nc(NC(=O)Cc4ccc5c(cnn5C)n4)cc3c2O[C@@H]1C] | LCMS-1 | 473.2 | 0.45 |
| I-379 | 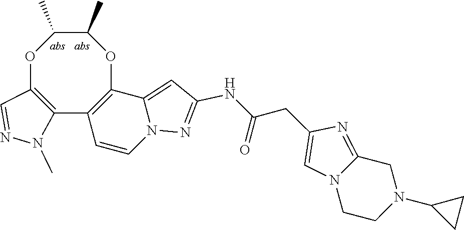 [SMILES: C[C@H]1Oc2cnn(C)c2-c2ccn3nc(NC(=O)Cc4cn5c(n4)CN(C4CC4)CC5)cc3c2O[C@@H]1C] | LCMS-1 | 503.2 | 0.423 |
| I-380 | 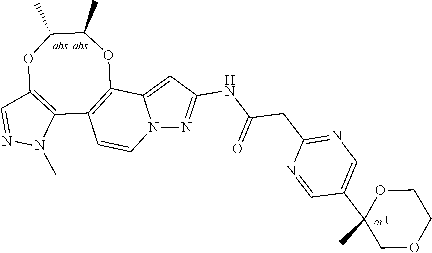 [SMILES: C[C@H]1Oc2cnn(C)c2-c2ccn3nc(NC(=O)Cc4ncc([C@@]5(C)COCCO5)cn4)cc3c2O[C@@H]1C] | LCMS-1 | 520.3 | 0.462 |
| I-381 | 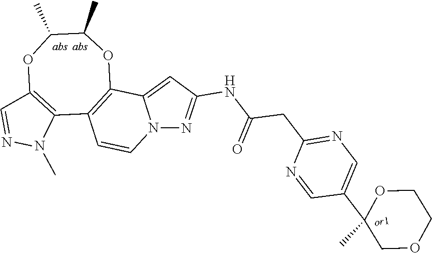 [SMILES: C[C@H]1Oc2cnn(C)c2-c2ccn3nc(NC(=O)Cc4ncc([C@]5(C)COCCO5)cn4)cc3c2O[C@@H]1C] | LCMS-1 | 520.3 | 0.463 |
| I-382 | 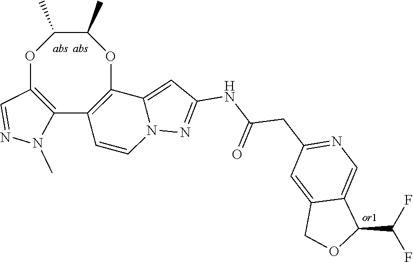 [SMILES: C[C@H]1Oc2cnn(C)c2-c2ccn3nc(NC(=O)Cc4cc5c(cn4)[C@@H](C(F)F)OC5)cc3c2O[C@@H]1C] | LCMS-1 | 511.3 | 0.461 |
| I-383 | 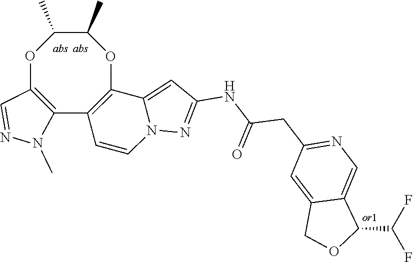 [SMILES: C[C@H]1Oc2cnn(C)c2-c2ccn3nc(NC(=O)Cc4cc5c(cn4)[C@H](C(F)F)OC5)cc3c2O[C@@H]1C] | LCMS-1 | 511.3 | 0.466 |
| I-384 | 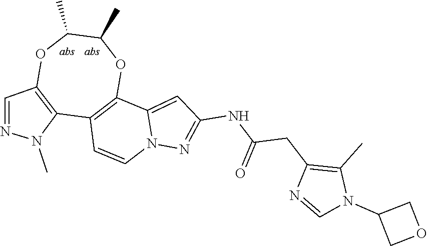 [SMILES: Cc1c(CC(=O)Nc2cc3c4c(ccn3n2)-c2c(cnn2C)O[C@H](C)[C@@H](C)O4)ncn1C1COC1] | LCMS-1 | 478.3 | 0.404 |
| I-385 | 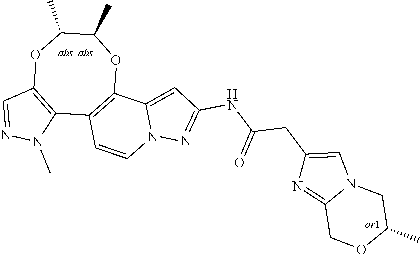 [SMILES: C[C@H]1Cn2cc(CC(=O)Nc3cc4c5c(ccn4n3)-c3c(cnn3C)O[C@H](C)[C@@H](C)O5)nc2CO1] | LCMS-1 | 478.2 | 0.417 |
| I-386 | 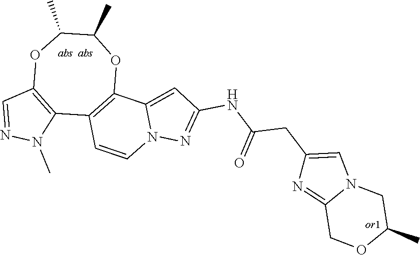 [SMILES: C[C@@H]1Cn2cc(CC(=O)Nc3cc4c5c(ccn4n3)-c3c(cnn3C)O[C@H](C)[C@@H](C)O5)nc2CO1] | LCMS-1 | 478.2 | 0.416 |
| I-387 | 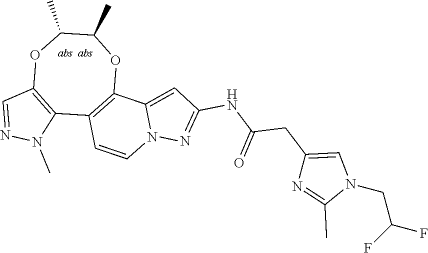 [SMILES: Cc1nc(CC(=O)Nc2cc3c4c(ccn3n2)-c2c(cnn2C)O[C@H](C)[C@@H](C)O4)cn1CC(F)F] | LCMS-1 | 486.2 | 0.413 |
| I-388 | 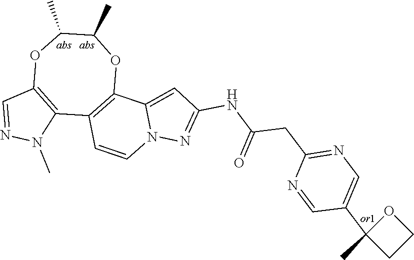 [SMILES: C[C@H]1Oc2cnn(C)c2-c2ccn3nc(NC(=O)Cc4ncc([C@@]5(C)CCO5)cn4)cc3c2O[C@@H]1C] | LCMS-1 | 490.2 | 0.451 |
| I-389 | 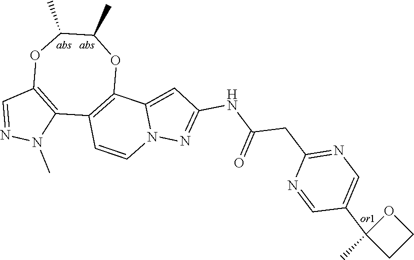 [SMILES: C[C@H]1Oc2cnn(C)c2-c2ccn3nc(NC(=O)Cc4ncc([C@]5(C)CCO5)cn4)cc3c2O[C@@H]1C] | LCMS-1 | 490.3 | 0.467 |
| I-390 | 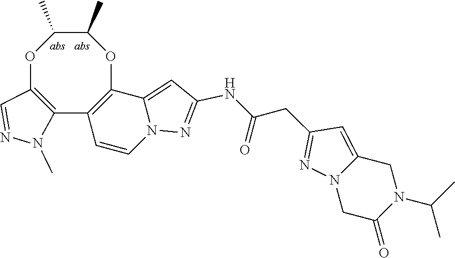 [SMILES: CC(C)N1Cc2cc(CC(=O)Nc3cc4c5c(ccn4n3)-c3c(cnn3C)O[C@H](C)[C@@H](C)O5)nn2CC1=O] | LCMS-1 | 519.2 | 0.464 |
| I-391 | 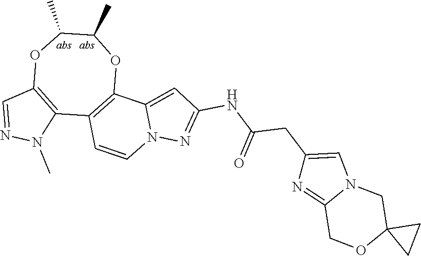 [SMILES: C[C@H]1Oc2cnn(C)c2-c2ccn3nc(NC(=O)Cc4cn5c(n4)COC4(CC4)C5)cc3c2O[C@@H]1C] | LCMS-1 | 490.3 | 0.421 |
| I-392 | 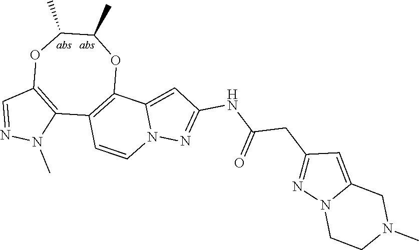 [SMILES: C[C@H]1Oc2cnn(C)c2-c2ccn3nc(NC(=O)Cc4cc5n(n4)CCN(C)C5)cc3c2O[C@@H]1C] | LCMS-1 | 477.2 | 0.758 |
| I-393 | 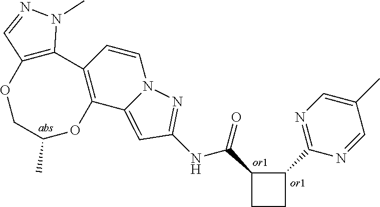 [SMILES: Cc1cnc([C@@H]2CC[C@H]2C(=O)Nc2cc3c4c(ccn3n2)-c2c(cnn2C)OC[C@@H](C)O4)nc1] | LCMS-1 | 460.1 | 0.481 |
| I-394 | 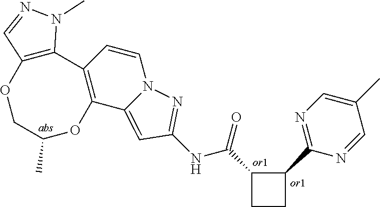 [SMILES: Cc1cnc([C@H]2CC[C@@H]2C(=O)Nc2cc3c4c(ccn3n2)-c2c(cnn2C)OC[C@@H](C)O4)nc1] | LCMS-1 | 460.2 | 0.484 |
| I-395 | 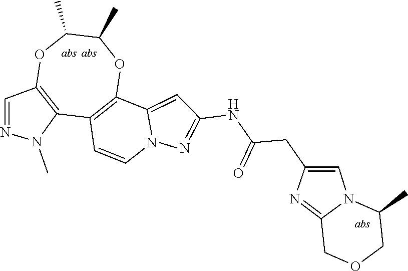 [SMILES: C[C@H]1Oc2cnn(C)c2-c2ccn3nc(NC(=O)Cc4cn5c(n4)COC[C@@H]5C)cc3c2O[C@@H]1C] | LCMS-1 | 478.2 | 0.442 |
| I-396 | 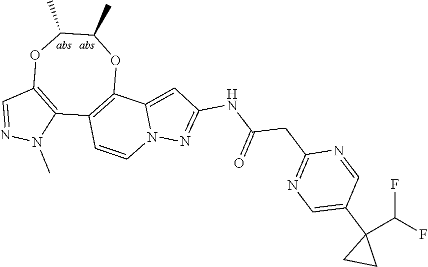 [SMILES: C[C@H]1Oc2cnn(C)c2-c2ccn3nc(NC(=O)Cc4ncc(C5(C(F)F)CC5)cn4)cc3c2O[C@@H]1C] | LCMS-2 | 510.2 | 0.577 |
| I-397 | 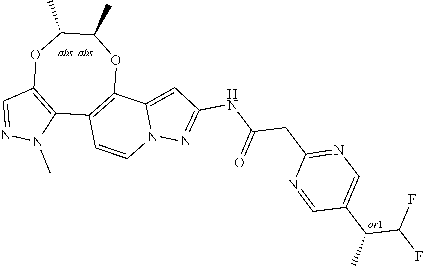 [SMILES: C[C@H](c1cnc(CC(=O)Nc2cc3c4c(ccn3n2)-c2c(cnn2C)O[C@H](C)[C@@H](C)O4)nc1)C(F)F] | LCMS-1 | 498.1 | 0.521 |
| I-398 | 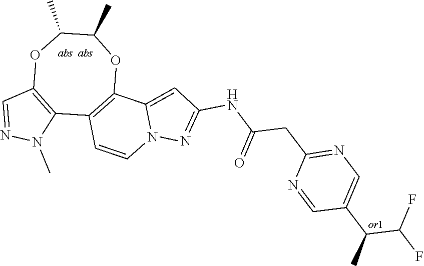 [SMILES: C[C@@H](c1cnc(CC(=O)Nc2cc3c4c(ccn3n2)-c2c(cnn2C)O[C@H](C)[C@@H](C)O4)nc1)C(F)F] | LCMS-1 | 498.1 | 0.529 |
| I-399 | 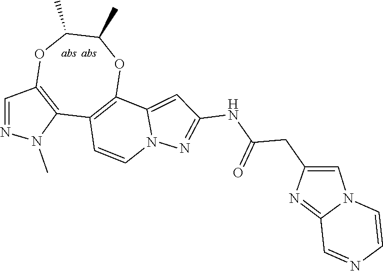 [SMILES: C[C@H]1Oc2cnn(C)c2-c2ccn3nc(NC(=O)Cc4cn5ccncc5n4)cc3c2O[C@@H]1C] | LCMS-1 | 459.2 | 0.41 |
| I-400 | 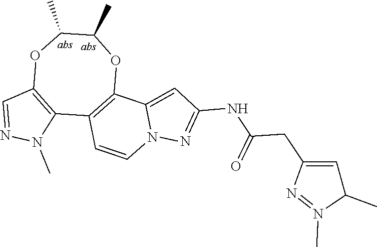 | LCMS-1 | 436.1 | 0.4 |
| I-401 | 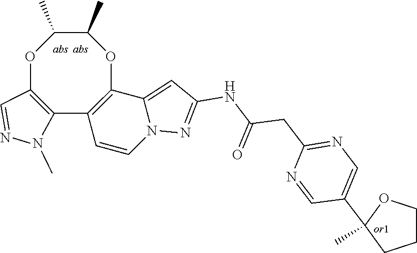 [SMILES: C[C@H]1Oc2cnn(C)c2-c2ccn3nc(NC(=O)Cc4ncc([C@]5(C)CCCO5)cn4)cc3c2O[C@@H]1C] | LCMS-1 | 504.2 | 0.513 |
| I-402 | 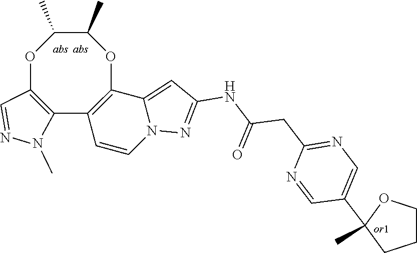 [SMILES: C[C@H]1Oc2cnn(C)c2-c2ccn3nc(NC(=O)Cc4ncc([C@@]5(C)CCCO5)cn4)cc3c2O[C@@H]1C] | LCMS-1 | 504.2 | 0.519 |
| I-403 | 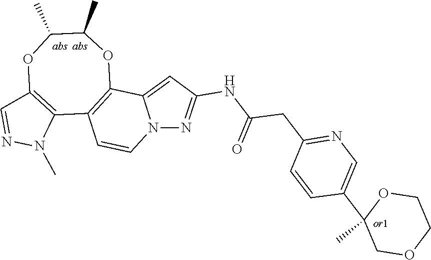 [SMILES: C[C@H]1Oc2cnn(C)c2-c2ccn3nc(NC(=O)Cc4ccc([C@]5(C)COCCO5)cn4)cc3c2O[C@@H]1C] | LCMS-1 | 519.2 | 0.435 |
| I-404 | 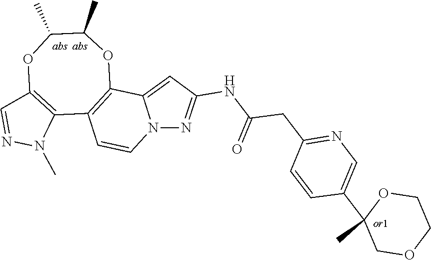 [SMILES: C[C@H]1Oc2cnn(C)c2-c2ccn3nc(NC(=O)Cc4ccc([C@@]5(C)COCCO5)cn4)cc3c2O[C@@H]1C] | LCMS-1 | 519.2 | 0.439 |
| I-405 | 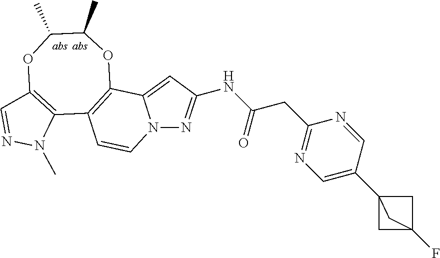 [SMILES: C[C@H]1Oc2cnn(C)c2-c2ccn3nc(NC(=O)Cc4ncc(C56CC(F)(C5)C6)cn4)cc3c2O[C@@H]1C] | LCMS-1 | 504.3 | 0.5 |
| I-406 | 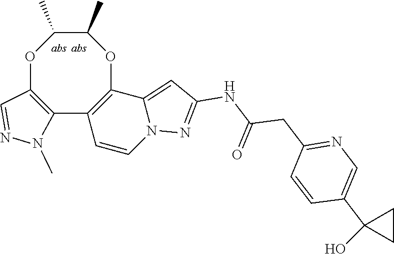 [SMILES: C[C@H]1Oc2cnn(C)c2-c2ccn3nc(NC(=O)Cc4ccc(C5(O)CC5)cn4)cc3c2O[C@@H]1C] | LCMS-1 | 475.1 | 0.443 |
| I-407 | 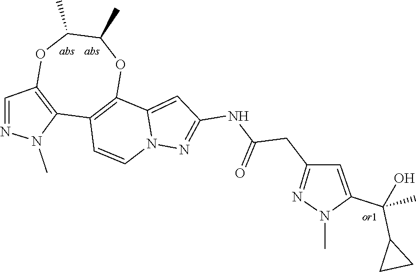 [SMILES: C[C@H]1Oc2cnn(C)c2-c2ccn3nc(NC(=O)Cc4cc([C@@](C)(O)C5CC5)n(C)n4)cc3c2O[C@@H]1C] | LCMS-2 | 506.3 | 0.875 |
| I-408 | 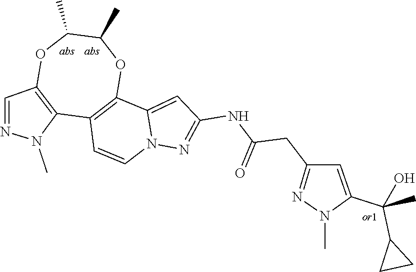 [SMILES: C[C@H]1Oc2cnn(C)c2-c2ccn3nc(NC(=O)Cc4cc([C@](C)(O)C5CC5)n(C)n4)cc3c2O[C@@H]1C] | LCMS-2 | 506.2 | 0.883 |
| I-409 | 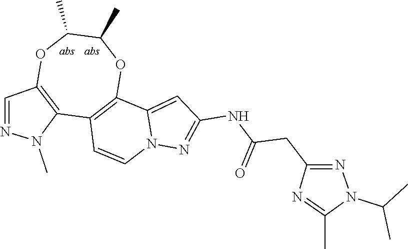 [SMILES: Cc1nc(CC(=O)Nc2cc3c4c(ccn3n2)-c2c(cnn2C)O[C@H](C)[C@@H](C)O4)nn1C(C)C] | LCMS-1 | 465.2 | 0.45 |
| I-410 | 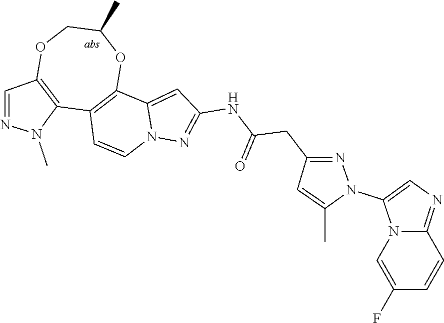 [SMILES: Cc1cc(CC(=O)Nc2cc3c4c(ccn3n2)-c2c(cnn2C)OC[C@@H](C)O4)nn1-c1cnc2ccc(F)cn12] | LCMS-2 | 542.2 | 0.846 |
| I-411 | 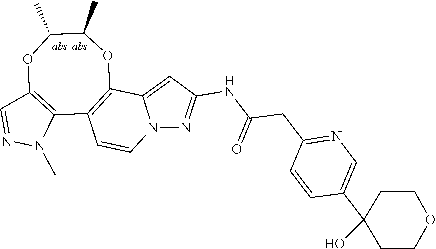 [SMILES: C[C@H]1Oc2cnn(C)c2-c2ccn3nc(NC(=O)Cc4ccc(C5(O)CCOCC5)cn4)cc3c2O[C@@H]1C] | LCMS-2 | 519.2 | 0.491 |
| I-412 | 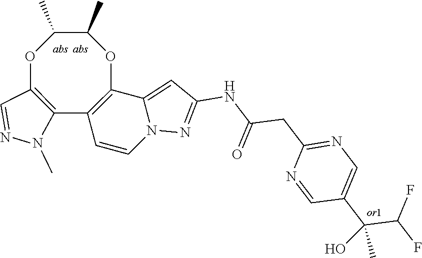 [SMILES: C[C@H]1Oc2cnn(C)c2-c2ccn3nc(NC(=O)Cc4ncc([C@](C)(O)C(F)F)cn4)cc3c2O[C@@H]1C] | LCMS-1 | 513.3 | 0.435 |
| I-413 | 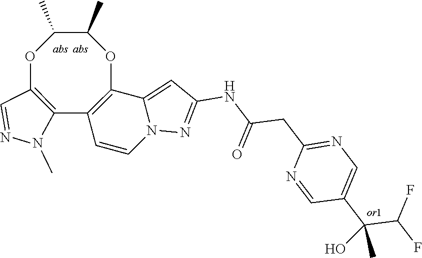 [SMILES: C[C@H]1Oc2cnn(C)c2-c2ccn3nc(NC(=O)Cc4ncc([C@@](C)(O)C(F)F)cn4)cc3c2O[C@@H]1C] | LCMS-1 | 513.3 | 0.436 |
| I-414 | 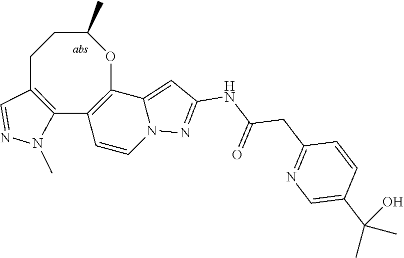 [SMILES: C[C@@H]1CCc2cnn(C)c2-c2ccn3nc(NC(=O)Cc4ccc(C(C)(C)O)cn4)cc3c2O1] | LCMS-1 | 461.1 | 0.444 |
| I-415 | 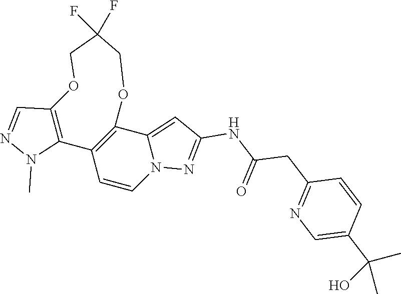 [SMILES: Cn1ncc2c1-c1ccn3nc(NC(=O)Cc4ccc(C(C)(C)O)cn4)cc3c1OCC(F)(F)CO2] | LCMS-1 | 499.2 | 0.414 |
| I-416 | 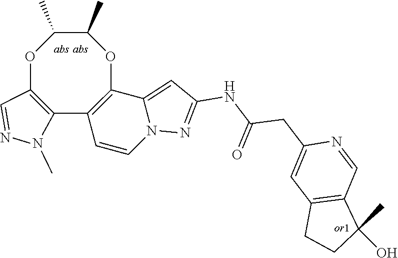 [SMILES: C[C@H]1Oc2cnn(C)c2-c2ccn3nc(NC(=O)Cc4cc5c(cn4)[C@](C)(O)CC5)cc3c2O[C@@H]1C] | LCMS-1 | 489.2 | 0.414 |
| I-417 | 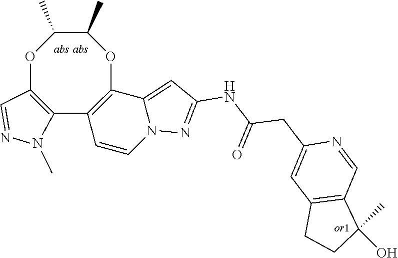 [SMILES: C[C@H]1Oc2cnn(C)c2-c2ccn3nc(NC(=O)Cc4cc5c(cn4)[C@@](C)(O)CC5)cc3c2O[C@@H]1C] | LCMS-1 | 489.2 | 0.416 |
| I-418 | 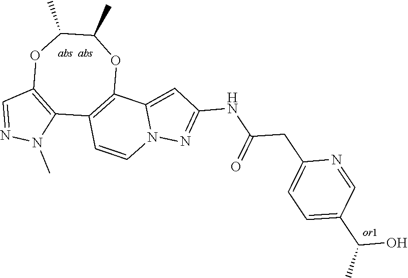 [SMILES: C[C@@H](O)c1ccc(CC(=O)Nc2cc3c4c(ccn3n2)-c2c(cnn2C)O[C@H](C)[C@@H](C)O4)nc1] | LCMS-1 | 463.2 | 0.414 |
| I-419 | 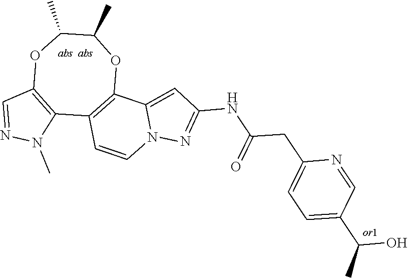 [SMILES: C[C@H](O)c1ccc(CC(=O)Nc2cc3c4c(ccn3n2)-c2c(cnn2C)O[C@H](C)[C@@H](C)O4)nc1] | LCMS-1 | 463.2 | 0.406 |
| I-420 | 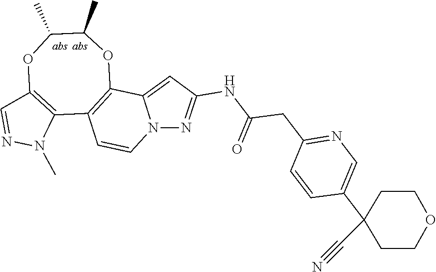 [SMILES: C[C@H]1Oc2cnn(C)c2-c2ccn3nc(NC(=O)Cc4ccc(C5(C#N)CCOCC5)cn4)cc3c2O[C@@H]1C] | LCMS-1 | 528.2 | 0.468 |
| I-421 | 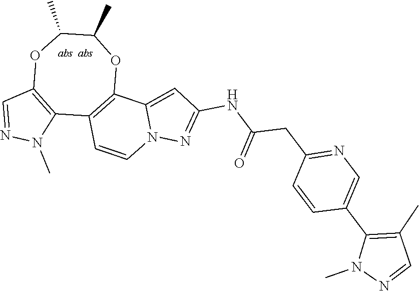 [SMILES: Cc1cnn(C)c1-c1ccc(CC(=O)Nc2cc3c4c(ccn3n2)-c2c(cnn2C)O[C@H](C)[C@@H](C)O4)nc1] | LCMS-1 | 513.2 | 0.467 |
| I-422 | 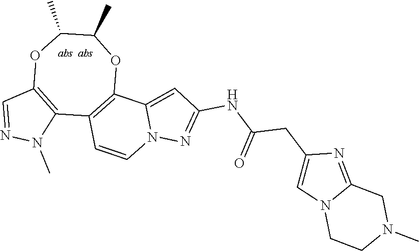 [SMILES: C[C@H]1Oc2cnn(C)c2-c2ccn3nc(NC(=O)Cc4cn5c(n4)CN(C)CC5)cc3c2O[C@@H]1C] | LCMS-1 | 477.3 | 0.395 |
| I-423 | 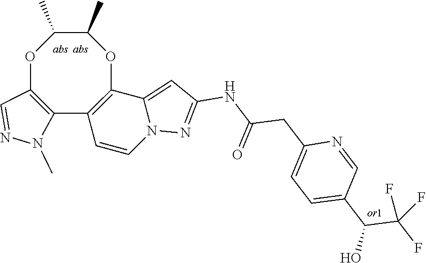 [SMILES: C[C@H]1Oc2cnn(C)c2-c2ccn3nc(NC(=O)Cc4ccc([C@@H](O)C(F)(F)F)cn4)cc3c2O[C@@H]1C] | LCMS-1 | 517.1 | 0.487 |
| I-424 | 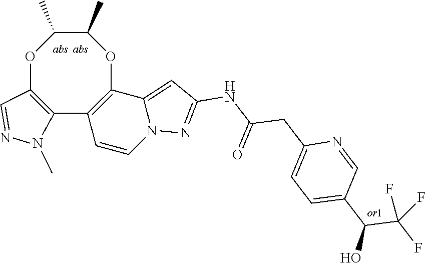 [SMILES: C[C@H]1Oc2cnn(C)c2-c2ccn3nc(NC(=O)Cc4ccc([C@H](O)C(F)(F)F)cn4)cc3c2O[C@@H]1C] | LCMS-1 | 517.1 | 0.493 |
| I-425 | 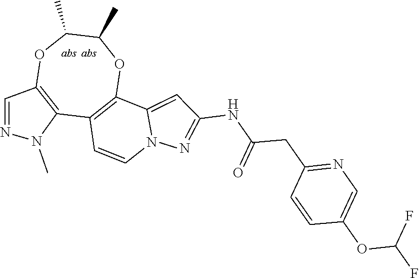 [SMILES: C[C@H]1Oc2cnn(C)c2-c2ccn3nc(NC(=O)Cc4ccc(OC(F)F)cn4)cc3c2O[C@@H]1C] | LCMS-1 | 485.3 | 0.499 |
| I-426 | 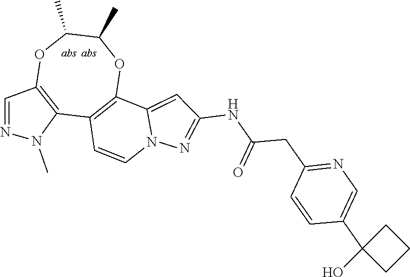 [SMILES: C[C@H]1Oc2cnn(C)c2-c2ccn3nc(NC(=O)Cc4ccc(C5(O)CCC5)cn4)cc3c2O[C@@H]1C] | LCMS-1 | 489.2 | 0.425 |
| I-427 | 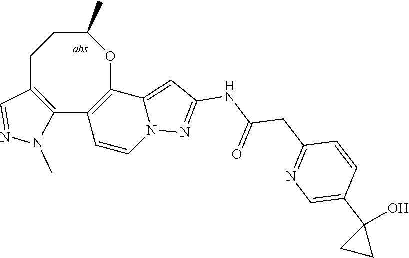 [SMILES: C[C@@H]1CCc2cnn(C)c2-c2ccn3nc(NC(=O)Cc4ccc(C5(O)CC5)cn4)cc3c2O1] | LCMS-1 | 459.2 | 0.4 |
| I-428 | 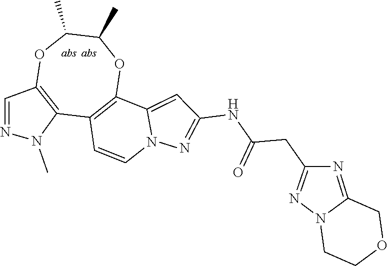 [SMILES: C[C@H]1Oc2cnn(C)c2-c2ccn3nc(NC(=O)Cc4nc5n(n4)CCOC5)cc3c2O[C@@H]1C] | LCMS-1 | 465.4 | 0.428 |
| I-429 | 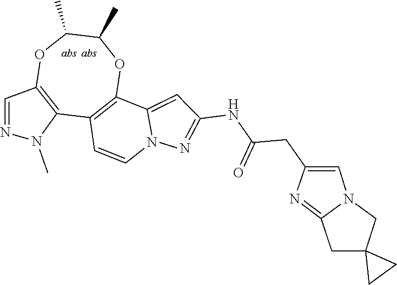 [SMILES: C[C@H]1Oc2cnn(C)c2-c2ccn3nc(NC(=O)Cc4cn5c(n4)CC4(CC4)C5)cc3c2O[C@@H]1C] | LCMS-1 | 474.2 | 0.417 |
| I-430 | 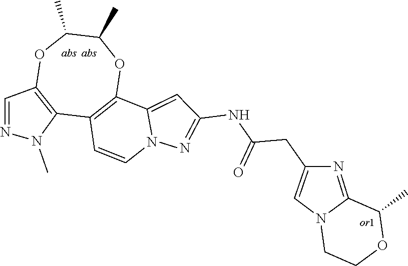 [SMILES: C[C@@H]1OCCn2cc(CC(=O)Nc3cc4c5c(ccn4n3)-c3c(cnn3C)O[C@H](C)[C@@H](C)O5)nc21] | LCMS-1 | 478.5 | 0.406 |
| I-431 | 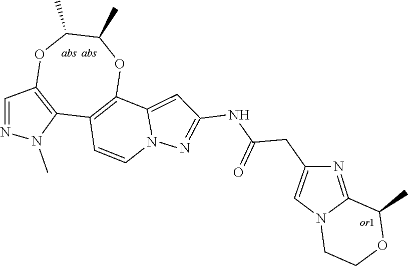 [SMILES: C[C@H]1OCCn2cc(CC(=O)Nc3cc4c5c(ccn4n3)-c3c(cnn3C)O[C@H](C)[C@@H](C)O5)nc21] | LCMS-1 | 478.4 | 0.409 |
| I-432 | 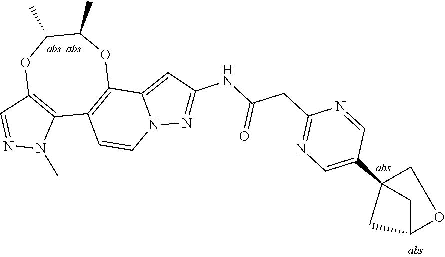 [SMILES: C[C@H]1Oc2cnn(C)c2-c2ccn3nc(NC(=O)Cc4ncc([C@]56CO[C@H](C5)C6)cn4)cc3c2O[C@@H]1C] | LCMS-1 | 502.5 | 0.448 |
| I-433 | 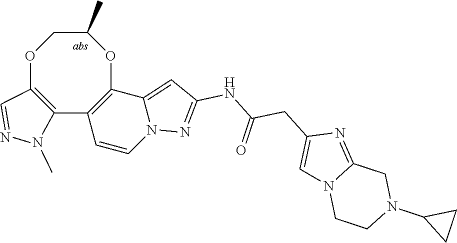 [SMILES: C[C@@H]1COc2cnn(C)c2-c2ccn3nc(NC(=O)Cc4cn5c(n4)CN(C4CC4)CC5)cc3c2O1] | LCMS-1 | 489.3 | 0.408 |
| I-434 | 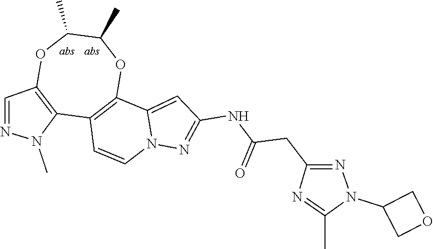 [SMILES: Cc1nc(CC(=O)Nc2cc3c4c(ccn3n2)-c2c(cnn2C)O[C@H](C)[C@@H](C)O4)nn1C1COC1] | LCMS-1 | 479.3 | 0.435 |
| I-435 | 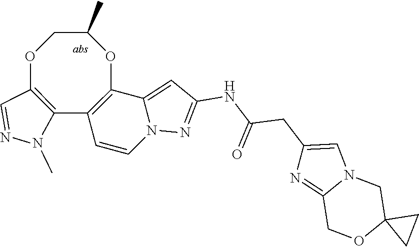 [SMILES: C[C@@H]1COc2cnn(C)c2-c2ccn3nc(NC(=O)Cc4cn5c(n4)COC4(CC4)C5)cc3c2O1] | LCMS-1 | 476.2 | 0.405 |
| I-436 | 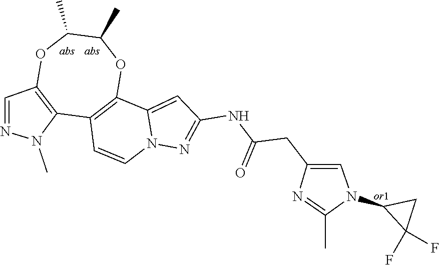 [SMILES: Cc1nc(CC(=O)Nc2cc3c4c(ccn3n2)-c2c(cnn2C)O[C@H](C)[C@@H](C)O4)cn1[C@H]1CC1(F)F] | LCMS-1 | 498.3 | 0.428 |
| I-437 | 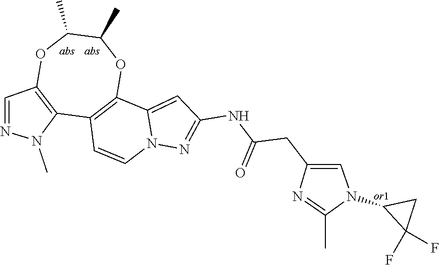 [SMILES: Cc1nc(CC(=O)Nc2cc3c4c(ccn3n2)-c2c(cnn2C)O[C@H](C)[C@@H](C)O4)cn1[C@@H]1CC1(F)F] | LCMS-1 | 498.3 | 0.425 |
| I-438 | 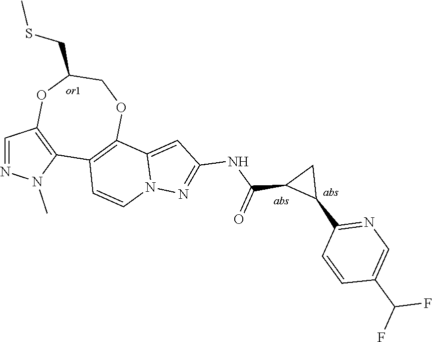 [SMILES: CSC[C@H]1COc2c(ccn3nc(NC(=O)[C@H]4C[C@H]4c4ccc(C(F)F)cn4)cc23)-c2c(cnn2C)O1] | LCMS-1 | 527.3 | 0.483 |
| I-439 | 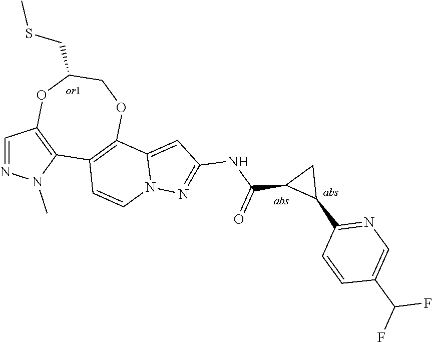 [SMILES: CSC[C@@H]1COc2c(ccn3nc(NC(=O)[C@H]4C[C@H]4c4ccc(C(F)F)cn4)cc23)-c2c(cnn2C)O1] | LCMS-1 | 527.3 | 0.482 |
| I-440 | 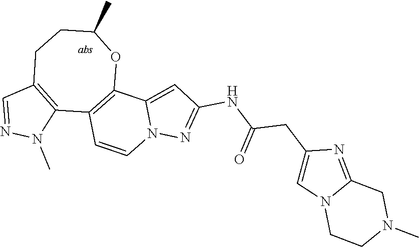 [SMILES: C[C@@H]1CCc2cnn(C)c2-c2ccn3nc(NC(=O)Cc4cn5c(n4)CN(C)CC5)cc3c2O1] | LCMS-1 | 461.3 | 0.396 |
| I-441 | 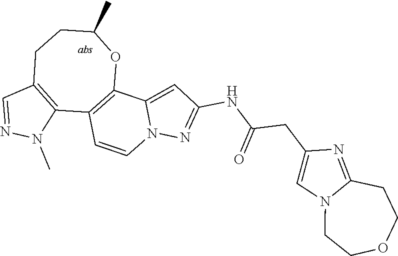 [SMILES: C[C@@H]1CCc2cnn(C)c2-c2ccn3nc(NC(=O)Cc4cn5c(n4)CCOCC5)cc3c2O1] | LCMS-1 | 462.2 | 0.405 |
| I-442 | 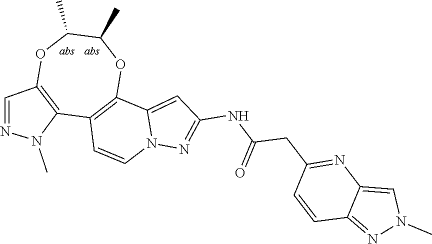 [SMILES: C[C@H]1Oc2cnn(C)c2-c2ccn3nc(NC(=O)Cc4ccc5nn(C)cc5n4)cc3c2O[C@@H]1C] | LCMS-1 | 473.3 | 0.445 |
| I-443 | 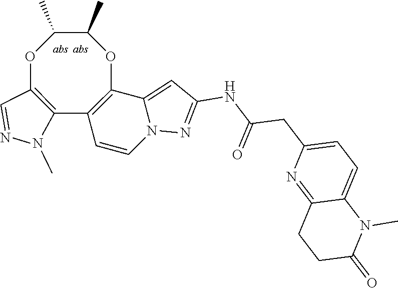 [SMILES: C[C@H]1Oc2cnn(C)c2-c2ccn3nc(NC(=O)Cc4ccc5c(n4)CCC(=O)N5C)cc3c2O[C@@H]1C] | LCMS-1 | 502.2 | 0.431 |
| I-444 | 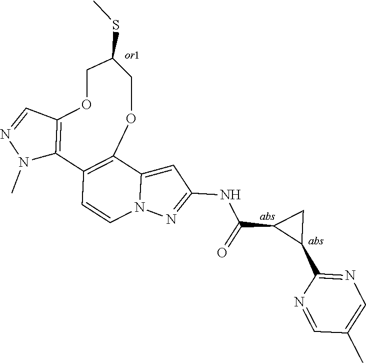 [SMILES: CS[C@@H]1COc2cnn(C)c2-c2ccn3nc(NC(=O)[C@H]4C[C@H]4c4ncc(C)cn4)cc3c2OC1] | LCMS-1 | 492.3 | 0.457 |
| I-445 | 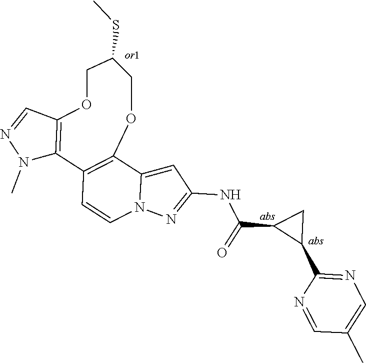 [SMILES: CS[C@H]1COc2cnn(C)c2-c2ccn3nc(NC(=O)[C@H]4C[C@H]4c4ncc(C)cn4)cc3c2OC1] | LCMS-1 | 492.2 | 0.459 |
| I-446 | 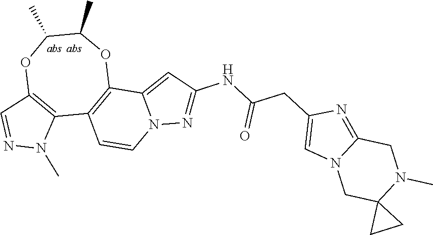 [SMILES: C[C@H]1Oc2cnn(C)c2-c2ccn3nc(NC(=O)Cc4cn5c(n4)CN(C)C4(CC4)C5)cc3c2O[C@@H]1C] | LCMS-1 | 503.3 | 0.509 |
| I-447 | 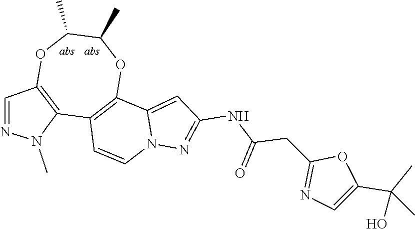 [SMILES: C[C@H]1Oc2cnn(C)c2-c2ccn3nc(NC(=O)Cc4ncc(C(C)(C)O)o4)cc3c2O[C@@H]1C] | LCMS-1 | 467.3 | 0.448 |
| I-448 | 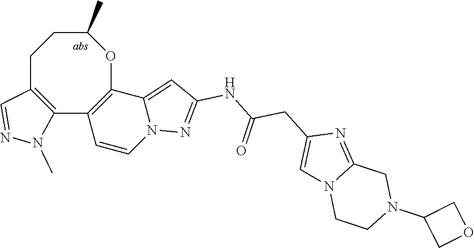 [SMILES: C[C@@H]1CCc2cnn(C)c2-c2ccn3nc(NC(=O)Cc4cn5c(n4)CN(C4COC4)CC5)cc3c2O1] | LCMS-1 | 503.2 | 0.397 |
| I-449 | 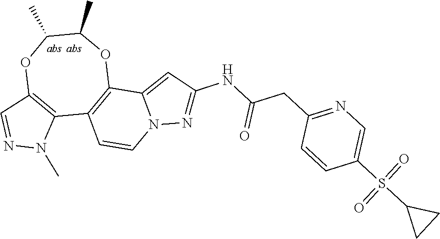 [SMILES: C[C@H]1Oc2cnn(C)c2-c2ccn3nc(NC(=O)Cc4ccc(S(=O)(=O)C5CC5)cn4)cc3c2O[C@@H]1C] | LCMS-1 | 523.1 | 0.475 |
| I-450 | 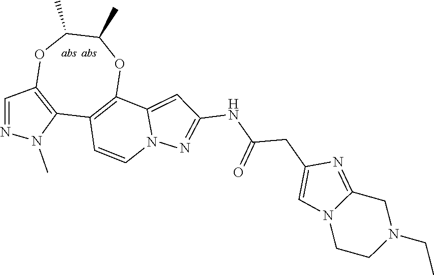 [SMILES: CCN1CCn2cc(CC(=O)Nc3cc4c5c(ccn4n3)-c3c(cnn3C)O[C@H](C)[C@@H](C)O5)nc2C1] | LCMS-1 | 491.3 | 0.392 |
| I-451 | 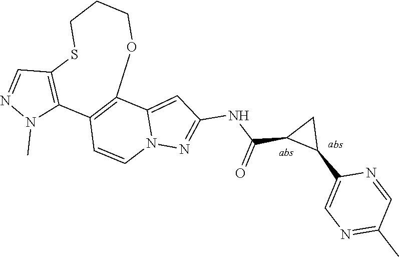 [SMILES: Cc1cnc([C@@H]2C[C@@H]2C(=O)Nc2cc3c4c(ccn3n2)-c2c(cnn2C)SCCCO4)cn1] | LCMS-1 | 462.2 | 0.453 |
| I-452 | 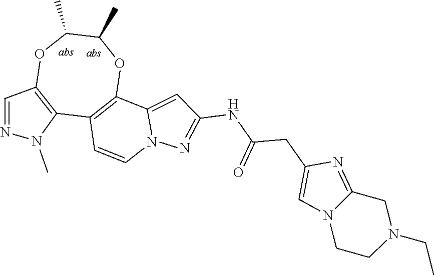 | LCMS-1 | 519.3 | 0.424 |
| I-453 | 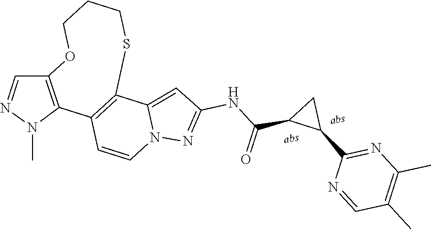 | LCMS-1 | 462.2 | 0.421 |
| I-454 | 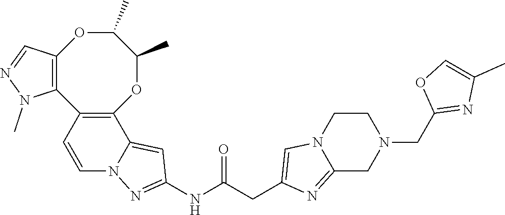 [SMILES: Cc1coc(CN2CCn3cc(CC(=O)Nc4cc5c6c(ccn5n4)-c4c(cnn4C)O[C@H](C)[C@@H](C)O6)nc3C2)n1] | LCMS-1 | 558.3 | 0.418 |
| I-455 | 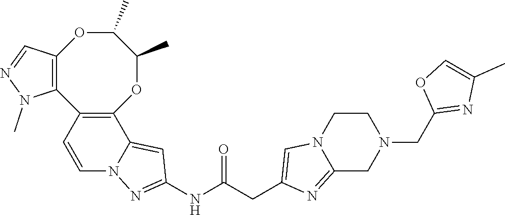 | LCMS-1 | 531.2 | 0.419 |
| I-456 | 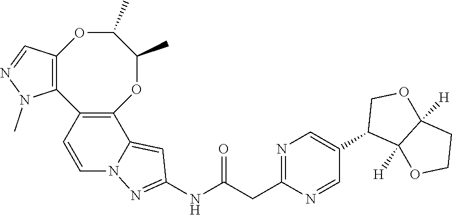 [SMILES: C[C@H]1Oc2cnn(C)c2-c2ccn3nc(NC(=O)Cc4ncc([C@@H]5CO[C@H]6CCO[C@H]65)cn4)cc3c2O[C@@H]1C] | LCMS-1 | 532.3 | 0.460 |
| I-457 | 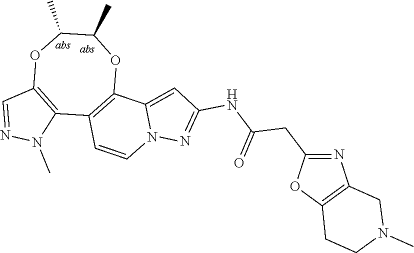 [SMILES: C[C@H]1Oc2cnn(C)c2-c2ccn3nc(NC(=O)Cc4nc5c(o4)CCN(C)C5)cc3c2O[C@@H]1C] | LCMS-1 | 478.2 | 0.393 |
| I-458 | 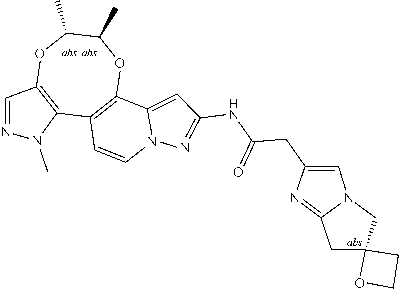 [SMILES: C[C@H]1Oc2cnn(C)c2-c2ccn3nc(NC(=O)Cc4cn5c(n4)C[C@]4(CCO4)C5)cc3c2O[C@@H]1C] | | | |
| I-459 | 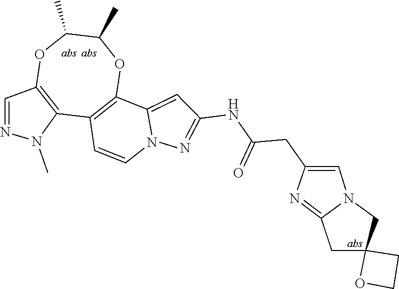 [SMILES: C[C@H]1Oc2cnn(C)c2-c2ccn3nc(NC(=O)Cc4cn5c(n4)C[C@@]4(CCO4)C5)cc3c2O[C@@H]1C] | | | |
| I-460 | 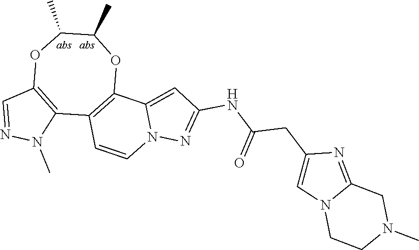 [SMILES: C[C@H]1Oc2cnn(C)c2-c2ccn3nc(NC(=O)Cc4cn5c(n4)CN(C)CC5)cc3c2O[C@@H]1C] | | | |
| I-461 | 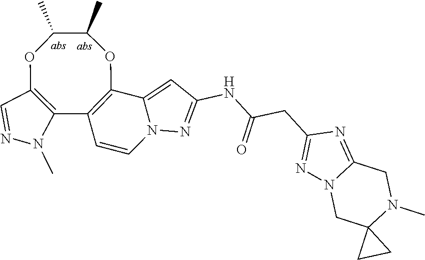 [SMILES: C[C@H]1Oc2cnn(C)c2-c2ccn3nc(NC(=O)Cc4nc5n(n4)CC4(CC4)N(C)C5)cc3c2O[C@@H]1C] | | | |
| I-462 | 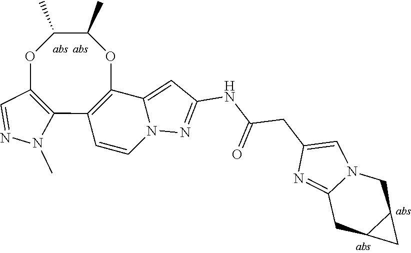 [SMILES: C[C@H]1Oc2cnn(C)c2-c2ccn3nc(NC(=O)Cc4cn5c(n4)C[C@@H]4C[C@@H]4C5)cc3c2O[C@@H]1C] | LCMS-1 | 474.3 | 0.418 |
| I-463 | 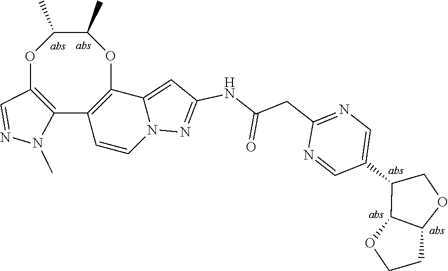 [SMILES: C[C@H]1Oc2cnn(C)c2-c2ccn3nc(NC(=O)Cc4ncc([C@@H]5CO[C@@H]6CCO[C@@H]65)cn4)cc3c2O[C@@H]1C] | | | |
| I-464 | 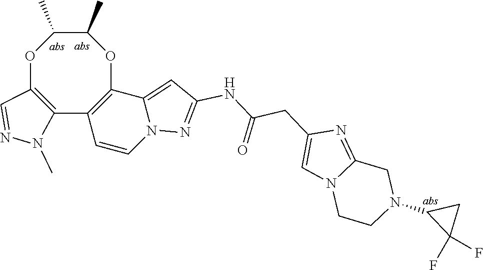 | | | |
| I-465 | 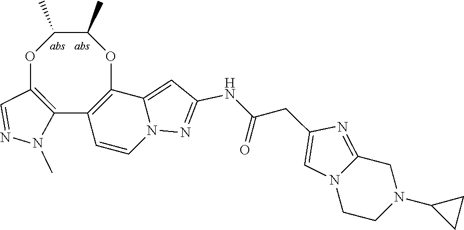 [SMILES: C[C@H]1Oc2cnn(C)c2-c2ccn3nc(NC(=O)Cc4cn5c(n4)CN(C4CC4)CC5)cc3c2O[C@@H]1C] | | | |
| I-466 | 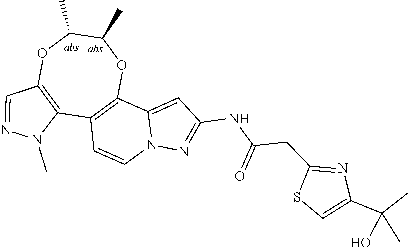 [SMILES: C[C@H]1Oc2cnn(C)c2-c2ccn3nc(NC(=O)Cc4nc(C(C)(C)O)cs4)cc3c2O[C@@H]1C] | | | |
| I-467 | 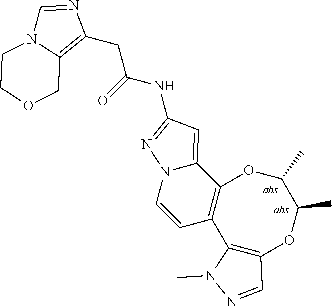 [SMILES: C[C@H]1Oc2cnn(C)c2-c2ccn3nc(NC(=O)Cc4ncn5c4COCC5)cc3c2O[C@@H]1C] | | | |
| I-468 | 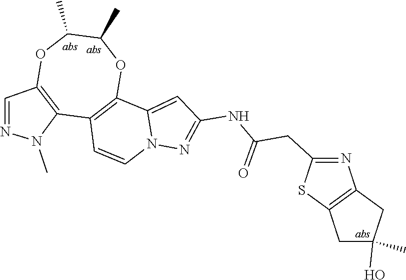 [SMILES: C[C@H]1Oc2cnn(C)c2-c2ccn3nc(NC(=O)Cc4nc5c(s4)C[C@](C)(O)C5)cc3c2O[C@@H]1C] | | | |
| I-469 | 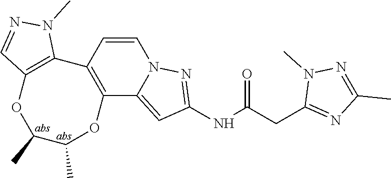 [SMILES: Cc1nc(CC(=O)Nc2cc3c4c(ccn3n2)-c2c(cnn2C)O[C@H](C)[C@@H](C)O4)n(C)n1] | | | |
| I-470 | 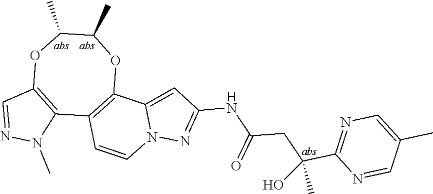 [SMILES: Cc1cnc([C@@](C)(O)CC(=O)Nc2cc3c4c(ccn3n2)-c2c(cnn2C)O[C@H](C)[C@@H](C)O4)nc1] | | | |
| I-471 | 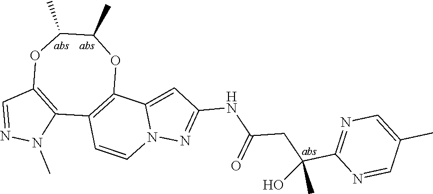 [SMILES: Cc1cnc([C@](C)(O)CC(=O)Nc2cc3c4c(ccn3n2)-c2c(cnn2C)O[C@H](C)[C@@H](C)O4)nc1] | | | |
| I-472 | 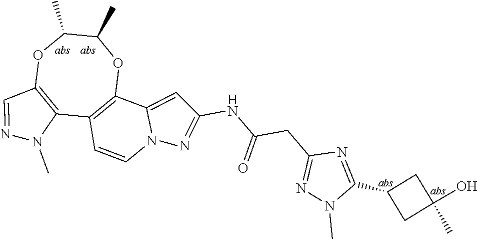 [SMILES: C[C@H]1Oc2cnn(C)c2-c2ccn3nc(NC(=O)Cc4nc([C@H]5C[C@](C)(O)C5)n(C)n4)cc3c2O[C@@H]1C] | | | |
| I-473 | 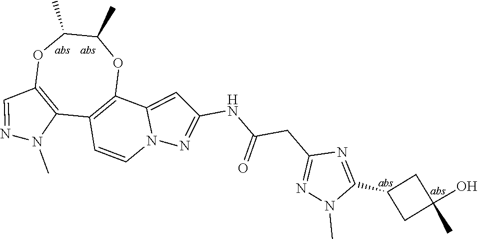 [SMILES: C[C@H]1Oc2cnn(C)c2-c2ccn3nc(NC(=O)Cc4nc([C@H]5C[C@@](C)(O)C5)n(C)n4)cc3c2O[C@@H]1C] | | | |
| I-474 | 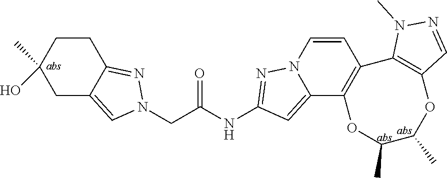 [SMILES: C[C@H]1Oc2cnn(C)c2-c2ccn3nc(NC(=O)Cn4cc5c(n4)CC[C@](C)(O)C5)cc3c2O[C@@H]1C] | | | |
| I-475 | 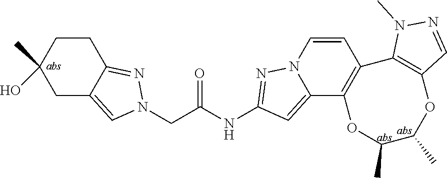 [SMILES: C[C@H]1Oc2cnn(C)c2-c2ccn3nc(NC(=O)Cn4cc5c(n4)CC[C@@](C)(O)C5)cc3c2O[C@@H]1C] | | | |
| I-476 | 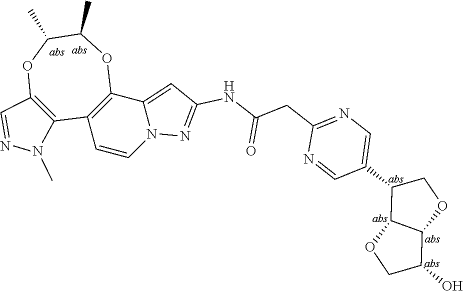 [SMILES: C[C@H]1Oc2cnn(C)c2-c2ccn3nc(NC(=O)Cc4ncc([C@@H]5CO[C@H]6[C@@H]5OC[C@H]6O)cn4)cc3c2O[C@@H]1C] | | | |
| I-477 | 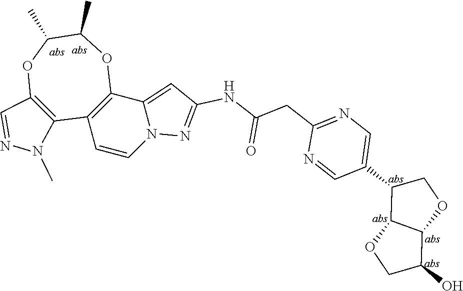 [SMILES: C[C@H]1Oc2cnn(C)c2-c2ccn3nc(NC(=O)Cc4ncc([C@@H]5CO[C@H]6[C@@H]5OC[C@@H]6O)cn4)cc3c2O[C@@H]1C] | | | |
| I-478 | 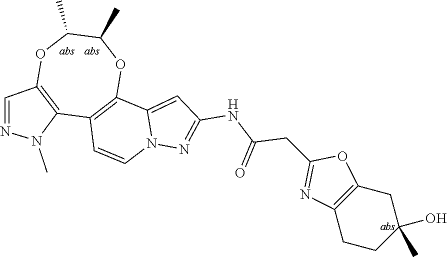 [SMILES: C[C@H]1Oc2cnn(C)c2-c2ccn3nc(NC(=O)Cc4nc5c(o4)C[C@](C)(O)CC5)cc3c2O[C@@H]1C] | | | |
| I-479 | 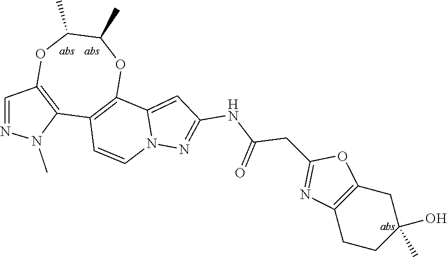 [SMILES: C[C@H]1Oc2cnn(C)c2-c2ccn3nc(NC(=O)Cc4nc5c(o4)C[C@@](C)(O)CC5)cc3c2O[C@@H]1C] | | | |
| I-480 | 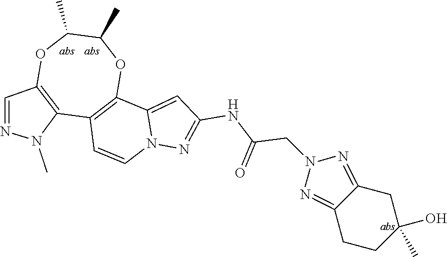 [SMILES: C[C@H]1Oc2cnn(C)c2-c2ccn3nc(NC(=O)Cn4nc5c(n4)C[C@@](C)(O)CC5)cc3c2O[C@@H]1C] | | | |
| I-481 | 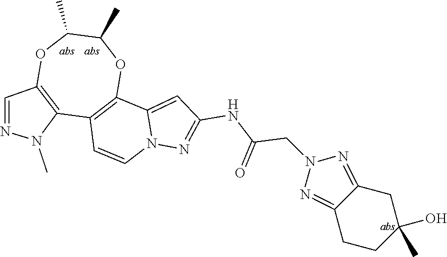 [SMILES: C[C@H]1Oc2cnn(C)c2-c2ccn3nc(NC(=O)Cn4nc5c(n4)C[C@](C)(O)CC5)cc3c2O[C@@H]1C] | | | |
| I-482 | 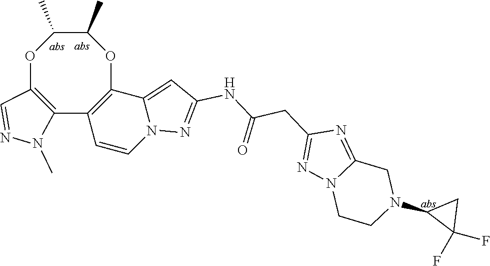 [SMILES: C[C@H]1Oc2cnn(C)c2-c2ccn3nc(NC(=O)Cc4nc5n(n4)CCN([C@H]4CC4(F)F)C5)cc3c2O[C@@H]1C] | | | |
| I-483 | 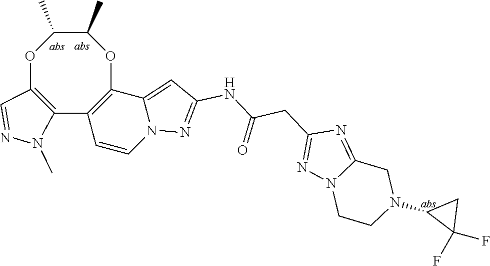 [SMILES: C[C@H]1Oc2cnn(C)c2-c2ccn3nc(NC(=O)Cc4nc5n(n4)CCN([C@@H]4CC4(F)F)C5)cc3c2O[C@@H]1C] | | | |
| I-484 | 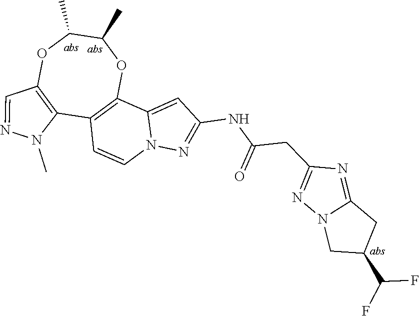 [SMILES: C[C@H]1Oc2cnn(C)c2-c2ccn3nc(NC(=O)Cc4nc5n(n4)C[C@H](C(F)F)C5)cc3c2O[C@@H]1C] | | | |
| I-485 | 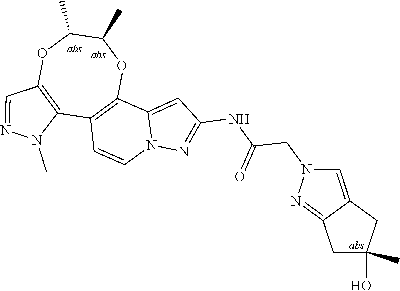 [SMILES: C[C@H]1Oc2cnn(C)c2-c2ccn3nc(NC(=O)Cn4cc5c(n4)C[C@@](C)(O)C5)cc3c2O[C@@H]1C] | | | |
| I-486 | 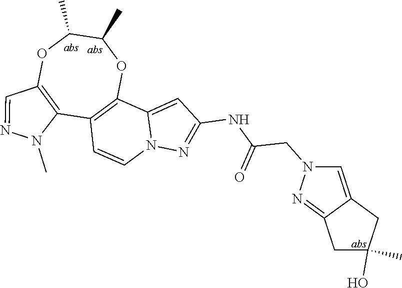 [SMILES: C[C@H]1Oc2cnn(C)c2-c2ccn3nc(NC(=O)Cn4cc5c(n4)C[C@](C)(O)C5)cc3c2O[C@@H]1C] | | | |
| I-487 | 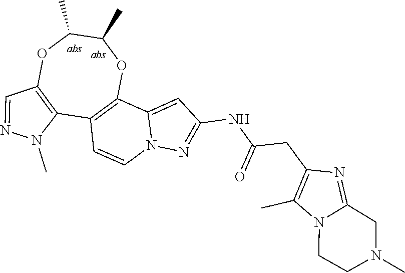 [SMILES: Cc1c(CC(=O)Nc2cc3c4c(ccn3n2)-c2c(cnn2C)O[C@H](C)[C@@H](C)O4)nc2n1CCN(C)C2] | | | |
| I-488 | 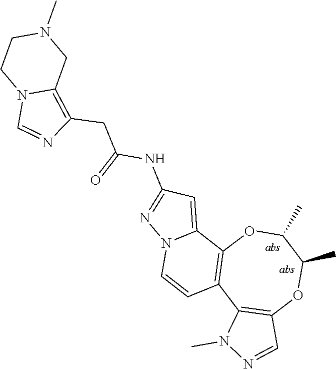 [SMILES: C[C@H]1Oc2cnn(C)c2-c2ccn3nc(NC(=O)Cc4ncn5c4CN(C)CC5)cc3c2O[C@@H]1C] | | | |
| I-489 | 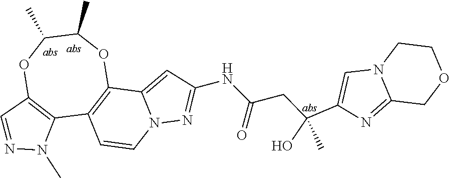 [SMILES: C[C@H]1Oc2cnn(C)c2-c2ccn3nc(NC(=O)C[C@](C)(O)c4cn5c(n4)COCC5)cc3c2O[C@@H]1C] | | | |
| I-490 | 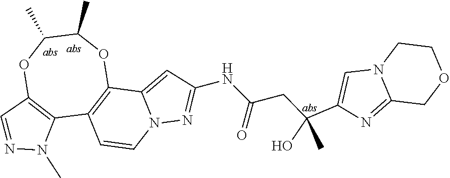 [SMILES: C[C@H]1Oc2cnn(C)c2-c2ccn3nc(NC(=O)C[C@@](C)(O)c4cn5c(n4)COCC5)cc3c2O[C@@H]1C] | | | |
| I-491 | 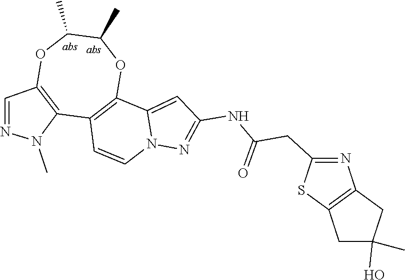 [SMILES: C[C@H]1Oc2cnn(C)c2-c2ccn3nc(NC(=O)Cc4nc5c(s4)CC(C)(O)C5)cc3c2O[C@@H]1C] | LCMS-1 | 495.2 | 0.467 |
| I-492 | 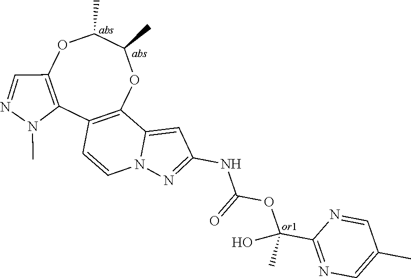 [SMILES: Cc1cnc([C@@](C)(O)OC(=O)Nc2cc3c4c(ccn3n2)-c2c(cnn2C)O[C@H](C)[C@@H](C)O4)nc1] | LCMS-1 | 478.2 | 0.457 |
| I-493 | 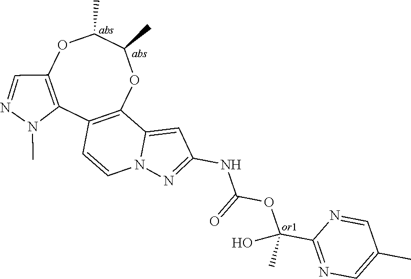 [SMILES: Cc1cnc([C@@](C)(O)OC(=O)Nc2cc3c4c(ccn3n2)-c2c(cnn2C)O[C@H](C)[C@@H](C)O4)nc1] | LCMS-1 | 478.3 | 0.459 |
| I-494 | 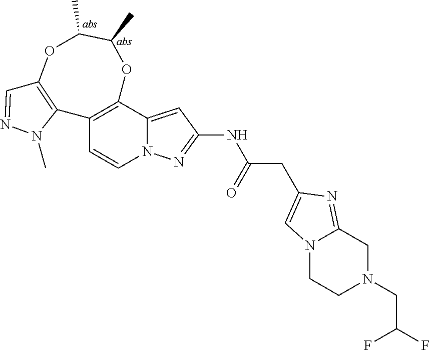 [SMILES: C[C@H]1Oc2cnn(C)c2-c2ccn3nc(NC(=O)Cc4cn5c(n4)CN(CC(F)F)CC5)cc3c2O[C@@H]1C] | | | |
| I-495 | 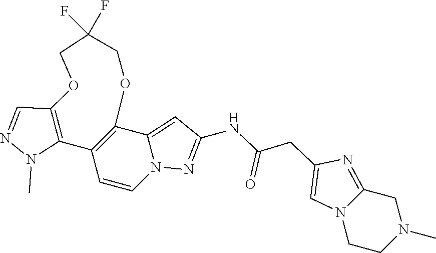 [SMILES: CN1CCn2cc(CC(=O)Nc3cc4c5c(ccn4n3)-c3c(cnn3C)OCC(F)(F)CO5)nc2C1] | | | |
| I-496 | 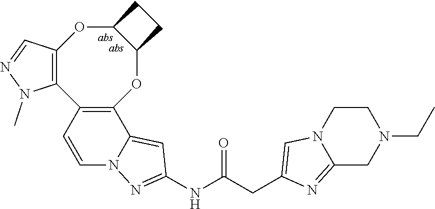 [SMILES: CCN1CCn2cc(CC(=O)Nc3cc4c5c(ccn4n3)-c3c(cnn3C)O[C@H]3CC[C@H]3O5)nc2C1] | | | |
| I-497 | 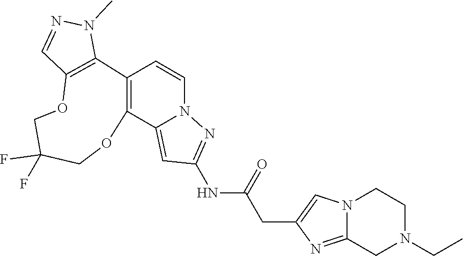 [SMILES: CCN1CCn2cc(CC(=O)Nc3cc4c5c(ccn4n3)-c3c(cnn3C)OCC(F)(F)CO5)nc2C1] | | | |
| I-498 | 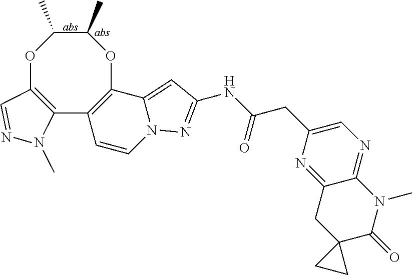 [SMILES: C[C@H]1Oc2cnn(C)c2-c2ccn3nc(NC(=O)Cc4cnc5c(n4)CC4(CC4)C(=O)N5C)cc3c2O[C@@H]1C] | | | |
| I-499 | 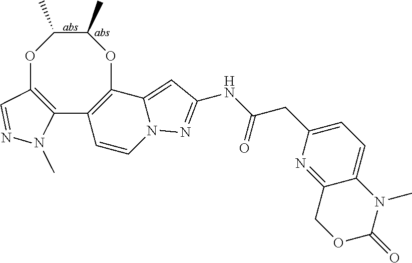 [SMILES: C[C@H]1Oc2cnn(C)c2-c2ccn3nc(NC(=O)Cc4ccc5c(n4)COC(=O)N5C)cc3c2O[C@@H]1C] | | | |
| I-500 | 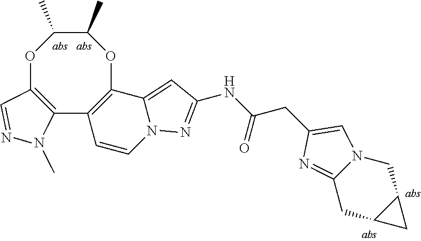 [SMILES: C[C@H]1Oc2cnn(C)c2-c2ccn3nc(NC(=O)Cc4cn5c(n4)C[C@H]4C[C@H]4C5)cc3c2O[C@@H]1C] | LCMS-1 | 474.2 | 0.423 |

### Example 23: Biochemical Assays

### SIK2-FLAG (1 mM ATP) ADP-Glo Assay Protocol (Assay A)

Reagents were procured and 1× assay buffer was prepared as above except for the AMARAtide substrate and the human recombinant SIK2 (SIK2-FLAG). AMARAtide was purchased from Biopeptide (San Diego, CA) and SIK2-FLAG (UniProt ID Q9H0K1) (G13-L350) was codon optimized for expression in SF21 cells and lysed in a buffer containing 50 mM Tris-HCl (pH 7.5), 500 mM NaCl, 5% glycerol, 0.5% Tween-20, 1 mM TCEP and purified over Glutathione Sepharose FF cartridge and Size Exclusion Chromatography (HiLoad 16/600 Superdex 200 pg) into a final storage buffer of 50 mM Tris-HCl (pH 7.5), 300 mM NaCl, 10% glycerol, 1 mM TCEP. The final protein, stored at −80° C., contains a single Glycine cloning artifact at the N-terminus and retains a c-terminal FLAG tag and consists of an amino acid sequence of SEQ ID NO: 1. Compounds (60 nL) in 100% DMSO were added to the assay plate (AlphaPlate-384, Cat #6005359 Perkin Elmer) using a LabCyte Echo 550. Similarly, DMSO (60 nL) was added to maximum signal control (MAX) wells and a reference compound producing 100% inhibition was added to minimum signal control (MIN) wells. SIK2 was diluted to 2× final concentration in 1× assay buffer and 3 μL was added to the compounds/DMSO in the assay plate. After incubation for 20 minutes at RT, 3 μL substrate mix containing 2×ATP and 2×AMARAtide in 1× assay buffer was added to the assay plate. After incubation for 60 minutes, the reaction was stopped by the addition of the ADP-Glo Kit components as directed and the relative luminescence units (RLU) were read on an Envision 2104. The final concentrations of the assay components were 3 nM SIK2-FLAG, 50 μM AMARAtide, 1 mM ATP, and 1% DMSO.

### Inhibition Calculation:

$$\% INH = ( RLU MAX - RLU sample ) / ( RLU MAX - RLU MIN ) \times 100$$

Where RLU=relative luminescence units, sample=signal in sample well, and MIN and MAX are the minimum and maximum signal controls, respectively.

### Four-Parameter IC₅₀ Fit:

$$Y = Bottom + ( Top - Bottom ) / ( 1 + ( I {C}_{50} / X ) \hat{} Hill Slope$$

Where top and bottom are the normally allowed to float, but may be fixed at 100 or 0, respectively, in a 3-parameter fit. Y is the % inhibition and X is the compound concentration.

Table 3 shows the IC₅₀ of selected compounds of this invention in the above assays. Compound activity designations have the following values: A (<10 nM); B (10 nM to <100 nM); C (100 nM to <500 nM); D (>500 nM); and ** (not tested).

TABLE 3
Biochemical Assays Results

| I-# | Assay A |
|---|---|
| I-1 | A |
| I-2 | A |
| I-3 | B |
| I-4 | A |
| I-5 | A |
| I-6 | A |
| I-7 | B |
| I-8 | B |
| I-9 | B |
| I-10 | B |
| I-11 | C |
| I-12 | C |
| I-13 | B |
| I-14 | A |
| I-15 | A |
| I-16 | A |
| I-17 | B |
| I-18 | A |
| I-19 | A |
| I-20 | B |
| I-21 | B |
| I-22 | B |
| I-23 | C |
| I-24 | B |
| I-25 | B |
| I-26 | B |
| I-27 | B |
| I-28 | B |
| I-29 | C |
| I-30 | C |
| I-31 | C |
| I-32 | C |
| I-33 | B |
| I-34 | B |
| I-35 | C |
| I-36 | C |
| I-37 | C |
| I-38 | B |
| I-39 | C |
| I-40 | C |
| I-41 | C |
| I-42 | A |
| I-43 | A |
| I-44 | D |
| I-45 | B |
| I-46 | B |
| I-47 | A |
| I-48 | B |
| I-49 | A |
| I-50 | B |
| I-51 | A |
| I-52 | D |
| I-53 | A |
| I-54 | A |
| I-55 | A |
| I-56 | A |
| I-57 | A |
| I-58 | A |
| I-59 | D |
| I-60 | B |
| I-61 | A |
| I-62 | A |
| I-63 | A |
| I-64 | B |
| I-65 | B |
| I-66 | A |
| I-67 | A |
| I-68 | C |
| I-69 | D |
| I-70 | B |
| I-71 | A |
| I-72 | B |
| I-73 | A |
| I-74 | A |
| I-75 | A |
| I-76 | A |
| I-77 | A |
| I-78 | C |
| I-79 | A |
| I-80 | C |
| I-81 | A |
| I-82 | A |
| I-83 | A |
| I-84 | B |
| I-85 | A |
| I-86 | C |
| I-87 | A |
| I-88 | B |
| I-89 | C |
| I-90 | A |
| I-91 | A |
| I-92 | A |
| I-93 | B |
| I-94 | A |
| I-95 | A |
| I-96 | B |
| I-97 | A |
| I-98 | B |
| I-99 | A |
| I-100 | B |
| I-101 | A |
| I-102 | A |
| I-103 | B |
| I-104 | A |
| I-105 | A |
| I-106 | A |
| I-107 | B |
| I-108 | A |
| I-109 | A |
| I-110 | B |
| I-111 | B |
| I-112 | B |
| I-113 | A |
| I-114 | B |
| I-115 | A |
| I-116 | A |
| I-117 | A |
| I-118 | A |
| I-119 | A |
| I-120 | A |
| I-121 | D |
| I-122 | C |
| I-123 | A |
| I-124 | B |
| I-125 | A |
| I-126 | C |
| I-127 | B |
| I-128 | A |
| I-129 | A |
| I-130 | B |
| I-131 | A |
| I-132 | A |
| I-133 | A |
| I-134 | A |
| I-135 | B |
| I-136 | A |
| I-137 | B |
| I-138 | B |
| I-139 | A |
| I-140 | A |
| I-141 | A |
| I-142 | B |
| I-143 | C |
| I-144 | A |
| I-145 | C |
| I-146 | B |
| I-147 | A |
| I-148 | C |
| I-149 | A |
| I-150 | A |
| I-151 | B |
| I-152 | A |
| I-153 | A |
| I-154 | A |
| I-155 | A |
| I-156 | A |
| I-157 | A |
| I-158 | A |
| I-159 | A |
| I-160 | B |
| I-161 | C |
| I-162 | B |
| I-163 | A |
| I-164 | C |
| I-165 | A |
| I-166 | A |
| I-167 | A |
| I-168 | B |
| I-169 | B |
| I-170 | C |
| I-171 | A |
| I-172 | C |
| I-173 | A |
| I-174 | B |
| I-175 | A |
| I-176 | A |
| I-177 | A |
| I-178 | A |
| I-179 | C |
| I-180 | A |
| I-181 | A |
| I-182 | A |
| I-183 | A |
| I-184 | A |
| I-185 | A |
| I-186 | A |
| I-187 | A |
| I-188 | A |
| I-189 | A |
| I-190 | A |
| I-191 | A |
| I-192 | C |
| I-193 | B |
| I-194 | A |
| I-195 | A |
| I-196 | A |
| I-197 | A |
| I-198 | A |
| I-199 | A |
| I-200 | C |
| I-201 | B |
| I-202 | A |
| I-203 | A |
| I-204 | A |
| I-205 | A |
| I-206 | A |
| I-207 | A |
| I-208 | A |
| I-209 | A |
| I-210 | A |
| I-211 | C |
| I-212 | B |
| I-213 | B |
| I-214 | A |
| I-215 | C |
| I-216 | A |
| I-217 | C |
| I-218 | B |
| I-219 | A |
| I-220 | A |
| I-221 | B |
| I-222 | A |
| I-223 | A |
| I-224 | A |
| I-225 | A |
| I-226 | C |
| I-227 | A |
| I-228 | A |
| I-229 | A |
| I-230 | B |
| I-231 | A |
| I-232 | A |
| I-233 | B |
| I-234 | B |
| I-235 | C |
| I-236 | B |
| I-237 | A |
| I-238 | B |
| I-239 | B |
| I-240 | B |
| I-241 | C |
| I-242 | A |
| I-243 | A |
| I-244 | C |
| I-245 | B |
| I-246 | C |
| I-247 | B |
| I-248 | A |
| I-249 | B |
| I-250 | A |
| I-251 | A |
| I-252 | B |
| I-253 | B |
| I-254 | C |
| I-255 | B |
| I-256 | C |
| I-257 | C |
| I-258 | A |
| I-259 | A |
| I-260 | B |
| I-261 | A |
| I-262 | A |
| I-263 | C |
| I-264 | B |
| I-265 | B |
| I-266 | A |
| I-267 | C |
| I-268 | A |
| I-269 | A |
| I-270 | A |
| I-271 | C |
| I-272 | A |
| I-273 | B |
| I-274 | C |
| I-275 | C |
| I-276 | B |
| I-277 | D |
| I-278 | D |
| I-279 | B |
| I-280 | B |
| I-281 | C |
| I-282 | B |
| I-283 | C |
| I-284 | A |
| I-285 | B |
| I-286 | A |
| I-287 | A |
| I-288 | A |
| I-289 | B |
| I-290 | B |
| I-291 | A |
| I-292 | B |
| I-293 | B |
| I-294 | B |
| I-295 | A |
| I-296 | C |
| I-297 | C |
| I-298 | B |
| I-299 | C |
| I-300 | B |
| I-301 | B |
| I-302 | B |
| I-303 | C |
| I-304 | B |
| I-305 | B |
| I-306 | B |
| I-307 | A |
| I-308 | A |
| I-309 | A |
| I-310 | D |
| I-311 | B |
| I-312 | A |
| I-313 | A |
| I-314 | D |
| I-315 | C |
| I-316 | B |
| I-317 | B |
| I-318 | B |
| I-319 | D |
| I-320 | D |
| I-321 | B |
| I-322 | C |
| I-323 | C |
| I-324 | B |
| I-325 | A |
| I-326 | B |
| I-327 | B |
| I-328 | C |
| I-329 | A |
| I-330 | B |
| I-331 | B |
| I-332 | A |
| I-333 | A |
| I-334 | A |
| I-335 | A |
| I-336 | A |
| I-337 | A |
| I-338 | C |
| I-339 | A |
| I-340 | A |
| I-341 | C |
| I-342 | B |
| I-343 | B |
| I-344 | B |
| I-345 | C |
| I-346 | A |
| I-347 | B |
| I-348 | C |
| I-349 | B |
| I-350 | B |
| I-351 | B |
| I-352 | C |
| I-353 | B |
| I-354 | B |
| I-355 | B |
| I-356 | A |
| I-357 | A |
| I-358 | B |
| I-359 | B |
| I-360 | B |
| I-361 | B |
| I-362 | C |
| I-363 | B |
| I-364 | B |
| I-365 | B |
| I-366 | B |
| I-367 | C |
| I-368 | B |
| I-369 | A |
| I-370 | A |
| I-371 | A |
| I-372 | A |
| I-373 | ** |
| I-374 | A |
| I-375 | A |
| I-376 | A |
| I-377 | B |
| I-378 | A |
| I-379 | A |
| I-380 | A |
| I-381 | B |
| I-382 | A |
| I-383 | B |
| I-384 | A |
| I-385 | A |
| I-386 | A |
| I-387 | A |
| I-388 | B |
| I-389 | B |
| I-390 | A |
| I-391 | A |
| I-392 | B |
| I-393 | C |
| I-394 | C |
| I-395 | A |
| I-396 | B |
| I-397 | B |
| I-398 | B |
| I-399 | B |
| I-400 | B |
| I-401 | B |
| I-402 | B |
| I-403 | A |
| I-404 | A |
| I-405 | A |
| I-406 | A |
| I-407 | A |
| I-408 | A |
| I-409 | A |
| I-410 | B |
| I-411 | A |
| I-412 | A |
| I-413 | A |
| I-414 | A |
| I-415 | B |
| I-416 | A |
| I-417 | B |
| I-418 | B |
| I-419 | B |
| I-420 | B |
| I-421 | A |
| I-422 | A |
| I-423 | A |
| I-424 | A |
| I-425 | B |
| I-426 | A |
| I-427 | A |
| I-428 | B |
| I-429 | A |
| I-430 | A |
| I-431 | A |
| I-432 | A |
| I-433 | A |
| I-434 | B |
| I-435 | A |
| I-436 | A |
| I-437 | A |
| I-438 | A |
| I-439 | B |
| I-440 | A |
| I-441 | A |
| I-442 | A |
| I-443 | A |
| I-444 | B |
| I-445 | A |
| I-446 | A |
| I-447 | B |
| I-448 | A |
| I-449 | A |
| I-450 | A |
| I-451 | A |
| I-452 | A |
| I-453 | B |
| I-454 | B |
| I-455 | A |
| I-456 | B |
| I-457 | B |
| I-462 | A |
| I-491 | A |
| I-500 | A |

While we have described a number of embodiments of this invention, it is apparent that our examples may be altered to provide other embodiments that utilize the compounds and methods of this invention. Therefore, it will be appreciated that the scope of this invention is to be defined by the appended claims rather than by the specific embodiments that have been represented by way of example.

1. A compound of formula I: 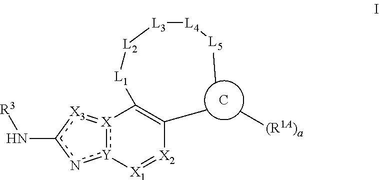
or a pharmaceutically acceptable salt thereof, wherein:
X is N or C;
Y is N or C, where when one of X and Y is N and the other of X and Y is C;
Each [symbol] is a single or double bond;
L₁ is selected from a bond, —O—, —S—, —S(O)₂—, —NR¹—, —C(O)—, —C(O)NR¹—, —NR¹C(O)—, —NR¹C(O)NR¹—, and —[C(R^{1a})(R^{1b})]₁₋₂—;
L₂ is selected from a bond, a substituted or unsubstituted alkenylene, —O—, —S—, —S(O)₂—, —N═S(O)(CH₃)—, —S(O)(CH₃)═N—, —S(O)₂NR¹—, —NR¹S(O)₂—, —NR¹—, —C(O)—, —C(O)NR¹—, —NR¹C(O)—, —NR¹C(O)NR¹—, an optionally substituted C₃-C₆ cycloalkyl, an optionally substituted C₃-C₆ heterocycloalkyl (having 1-4 heteroatoms independently selected from nitrogen, oxygen, and sulfur), an optionally substituted 5-6 membered monocyclic heteroaryl ring having 1-3 heteroatoms independently selected from nitrogen, oxygen, and sulfur; and —[C(R^{2a})(R^{2b})]₁₋₂—;
L₃ is selected from a bond, a substituted or unsubstituted alkenylene, —O—, —S—, —S(O)₂—, —N═S(O)(CH₃)—, —S(O)(CH₃)═N—, —S(O)₂NR¹—, —NR¹S(O)₂—, —NR¹—, —C(O)—, —C(O)NR¹—, —NR¹C(O)—, —NR¹C(O)NR¹—, an optionally substituted C₃-C₆ cycloalkyl, an optionally substituted C₃-C₆ heterocycloalkyl (having 1-4 heteroatoms independently selected from nitrogen, oxygen, and sulfur), an optionally substituted 5-6 membered monocyclic heteroaryl ring having 1-3 heteroatoms independently selected from nitrogen, oxygen, and sulfur; and —[C(R^{3a}) (R^{3b})]₁₋₂—;
L₄ is selected from a bond, a substituted or unsubstituted alkenylene, —O—, —S—, —S(O)₂—, —N═S(O)(CH₃)—, —S(O)(CH₃)═N—, —S(O)₂NR¹—, —NR¹S(O)₂—, —NR¹—, —C(O)—, —C(O)NR¹—, —NR¹C(O)—, —NR¹C(O)NR¹—, an optionally substituted C₃-C₆ cycloalkyl, an optionally substituted C₃-C₆ heterocycloalkyl (having 1-4 heteroatoms independently selected from nitrogen, oxygen, and sulfur), an optionally substituted 5-6 membered monocyclic heteroaryl ring having 1-3 heteroatoms independently selected from nitrogen, oxygen, and sulfur; and —[C(R^{4a})(R^{4b})]₁₋₂—;
L₅ is selected from a bond, —O—, —S—, —S(O)₂—, —NR¹—, —C(O)—, —C(O)NR¹—, —NR¹C(O)—, —NR¹C(O)NR¹—, and —[C(R^{5a})(R^{5b})]₁₋₂—;
Each R¹ is independently selected from the group consisting of hydrogen, C₁₋₃ haloalkyl, C₁₋₃ hydroxyalkyl, C₀₋₃ alkyl-C₃-C₆ cycloalkyl, C₀₋₃ alkyl-C₃-C₆ heterocycloalkyl (having 1-4 heteroatoms independently selected from nitrogen, oxygen, and sulfur), and C₁₋₃ alkyl; wherein C₀₋₃ alkyl-C₃-C₆ cycloalkyl, C₀₋₃ alkyl-C₃-C₆ heterocycloalkyl and C₁₋₃ alkyl are substituted with y instances of R^{B};
each R^{B} is independently halogen, C₁₋₃ alkyl, C₁₋₃ hydroxyalkyl, —CN, oxo, —OR, or —NR₂;
each y is independently 0, 1, 2, or 3;
R^{1a}, R^{1b}, R^{2a}, R^{2b}, R^{3a}, R^{3b}, R^{4a}, R^{4b}, R^{5a}, and R^{5b} are independently selected from the group consisting of hydrogen, halogen, hydroxyl, N(R¹)₂, C₁₋₃ alkyl, —CN, C₁₋₃ alkoxy, C₀₋₃ alkyl-C₃-C₆ cycloalkyl, C₀₋₃ alkyl-C₃-C₆ heterocycloalkyl, and C₁₋₃ hydroxyalkyl, and C₁₋₃ haloalkyl, wherein C₀₋₃ alkyl-C₃-C₆ cycloalkyl, C₀₋₃ alkyl-C₃-C₆ heterocycloalkyl and C₁₋₃ alkyl are substituted with y instances of R^{B}; or
R^{1a} and R^{1b}, R^{2a} and R^{2b}, R^{3a} and R^{3b}, R^{4a} and R^{4b}, or R^{5a} and R^{5b}, and the carbon atom to which they are attached may combine to form:
a 3-7 membered saturated or partially unsaturated bivalent monocyclic carbocyclylene or 4-7 membered saturated or partially unsaturated bivalent heterocyclylene ring (having 1-4 heteroatoms independently selected from nitrogen, oxygen, and sulfur, and 0 or 1 nitrogen atoms in addition to the 1-4 heteroatoms)—substituted with y instances of R^{B}, or
a 4-12 membered saturated or partially unsaturated bivalent bicyclic ring system that is fused, bridged, or spirocyclic selected from carbocyclylene or heterocyclylene (having 1-4 heteroatoms independently selected from nitrogen, oxygen, and sulfur)—substituted with y instances of R^{B}, or
R^{1a} and R^{2a}, R^{1a} and R^{3a}, R^{2a} and R^{3a}, R^{2a} and R^{4a}, R^{3a} and R^{4a}, R^{3a} and R^{5a}, or R^{4a} and R^{5a}, and the atoms to which they are connected may combine to form:
a 3-7 membered saturated or partially unsaturated bivalent monocyclic carbocyclylene or a 4-7 membered saturated or partially unsaturated bivalent heterocyclylene ring (having 1-4 heteroatoms independently selected from nitrogen, oxygen, and sulfur, and 0 or 1 nitrogen atoms in addition to the 1-4 heteroatoms)—substituted with y instances of R^{B}, or
a 4-12 membered saturated or partially unsaturated bivalent bicyclic ring system that is fused, bridged, or spirocyclic selected from carbocyclylene or heterocyclylene (having 1-4 heteroatoms independently selected from nitrogen, oxygen, and sulfur)—substituted with y instances of R^{B};
X₁ and X₂ are each selected from N and CR² provided when Y is N, X₁ is CR²;
each R² is independently selected from hydrogen, OH, C₁₋₃ alkyl, C₁₋₃ alkoxy, and halogen;
X₃ is selected from O, S, N, NR⁷, and CR², provided when X₃ is selected from O, S, and NR⁷, X and Y are C;
R⁷ is hydrogen or C₁₋₃ alkyl;
Ring C is a 5-6 membered monocyclic heteroaryl ring having 1-3 heteroatoms independently selected from nitrogen, oxygen, and sulfur; an 8-10 membered bicyclic heteroaryl ring having 1-4 heteroatoms independently selected from nitrogen, oxygen, and sulfur; or a 7-12 membered saturated or partially unsaturated bicyclic heterocyclyl ring having 1-4 heteroatoms independently selected from nitrogen, oxygen, and sulfur;
each R^{1A} is independently halogen, —CN, oxo, —OR, —SR, —NR₂, —SO₂R, —SO₂NR₂, —S(O)R, —S(O)NR₂, —C(O)R, —CO₂R, —C(O)NR₂, —C(O)N(R)OR, —OC(O)R, —OC(O)NR₂, —N(R)CO₂R, —N(R)C(O)R, —N(R)C(O)NR₂, —N(R)C(NR)R, —N(R)C(NR)NR₂, —N(R)NR₂, —N(R)SO₂NR₂, —N(R)SO₂R, —N═S(O)R₂, —S(NR)(O)R, —N(R)S(O)R, —N(R)CN, —P(O)(OR)₂, —P(O)R₂, C₁-C₆ haloalkyl, C₁₋₃ hydroxyalkyl, C₃-C₆ halocycloalkyl, C₁-C₆ haloalkoxy, or an optionally substituted group selected from C₁₋₆ aliphatic; phenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; and a 5-6 membered monocyclic heteroaryl ring having 1-4 heteroatoms independently selected from nitrogen, oxygen, and sulfur;
a is 0, 1, 2, or 3;
R³ is —C(O)-Le, —S(O)₂-Le, an optionally substituted C₁₋₆ aliphatic, an optionally substituted C₁₋₆ aliphatic substituted with 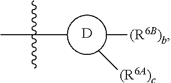
an optionally substituted 5-6 membered monocyclic heteroaromatic ring having 1-4 heteroatoms independently selected from nitrogen, oxygen, and sulfur, or 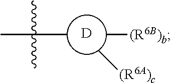
L₆ is selected from the group consisting of 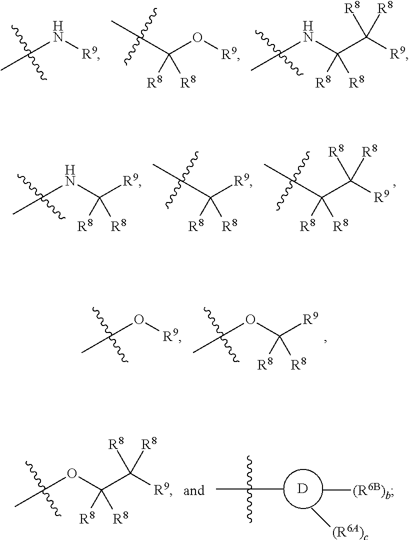
Ring D is a 3-8 membered saturated or partially unsaturated monocyclic carbocyclic, a 3-8 membered saturated or partially unsaturated monocyclic heterocyclic ring having 1-2 heteroatoms independently selected from nitrogen, oxygen, and sulfur, a 5-12 membered saturated or partially unsaturated bicyclic heterocyclic ring that is optionally bridged or spirocyclic having 1-4 heteroatoms independently selected from nitrogen, oxygen, and sulfur, or a 7-14 membered saturated or partially unsaturated tricyclic heterocyclic ring that is optionally bridged or spirocyclic having 1-4 heteroatoms independently selected from nitrogen, oxygen, and sulfur;
Each R⁸ is independently selected from hydrogen, halogen, hydroxyl, —CN, C₁₋₄ alkoxy, a 3-8 membered saturated or partially unsaturated monocyclic carbocyclic, C₁-C₆ haloalkyl, and C₁₋₄ alkyl; or two R⁸ groups and the atom to which they are attached can form oxo, a 3-8 membered saturated or partially unsaturated monocyclic carbocyclic ring or a 3-8 membered saturated or partially unsaturated monocyclic heterocyclic ring having 1-4 heteroatoms independently selected from nitrogen, oxygen, and sulfur;
R⁹ is selected from hydrogen, C₁-C₆ alkyl, C₁-C₆ haloalkyl, C₁₋₃ hydroxyalkyl, C₁-C₆ alkoxy, C₁-C₆ haloalkoxy, and an optionally substituted C₁₋₆ aliphatic; or
R⁹ is a cyclic group or an optionally substituted C₁₋₆ aliphatic substituted with a cyclic group, wherein the cyclic group is selected from:
phenyl;
a 3-8 membered saturated or partially unsaturated monocyclic carbocyclic ring;
a 3-8 membered saturated or partially unsaturated monocyclic heterocyclic ring having 1-2 heteroatoms independently selected from nitrogen, oxygen, and sulfur;
a 5-6 membered monocyclic heteroaromatic ring having 1-4 heteroatoms independently selected from nitrogen, oxygen, and sulfur;
an 8-10 membered bicyclic heteroaromatic ring having 1-5 heteroatoms independently selected from nitrogen, oxygen, and sulfur;
a 7-12 membered saturated or partially unsaturated bicyclic heterocyclic ring having 1-4 heteroatoms independently selected from nitrogen, oxygen, and sulfur;
a 5-12 membered saturated or partially unsaturated bicyclic carbocyclic ring that is optionally bridged or spirocyclic; and
a 5-12 membered saturated or partially unsaturated bicyclic heterocyclic ring that is optionally bridged or spirocyclic having 1-4 heteroatoms independently selected from nitrogen, oxygen, and sulfur,
wherein the cyclic group is substituted with y instances of R^{A};
each R^{6A} is independently a C₁₋₆ aliphatic-cyclic or cyclic group, wherein the cyclic group is selected from:
a 5-6 membered monocyclic heteroaromatic ring having 1-4 heteroatoms independently selected from nitrogen, oxygen, and sulfur;
an 8-10 membered bicyclic heteroaromatic ring having 1-5 heteroatoms independently selected from nitrogen, oxygen, and sulfur;
a 7-12 membered saturated or partially unsaturated bicyclic heterocyclic ring having 1-4 heteroatoms independently selected from nitrogen, oxygen, and sulfur;
a 5-12 membered saturated or partially unsaturated bicyclic carbocyclic ring that is optionally bridged or spirocyclic; and
a 5-12 membered saturated or partially unsaturated bicyclic heterocyclic ring that is optionally bridged or spirocyclic having 1-4 heteroatoms independently selected from nitrogen, oxygen, and sulfur,
wherein R^{6A} is substituted with y instances of R^{A};
each R^{6B} is independently halogen, —CN, oxo, —OR, —SR, —NR₂, —SO₂R, —SO₂NR₂, —S(O)R, —S(O)NR₂, —C(O)R, —CO₂R, —C(O)NR₂, —C(O)N(R)OR, —OC(O)R, —OC(O)NR₂, —N(R)CO₂R, —N(R)C(O)R, —N(R)C(O)NR₂, —N(R)C(NR)R, —N(R)C(NR)NR₂, —N(R)NR₂, —N(R)SO₂NR₂, —N(R)SO₂R, —N═S(O)R₂, —S(NR)(O)R, —N(R)S(O)R, —N(R)CN, —P(O)(OR)₂, —P(O)R₂, or an optionally substituted group selected from C₁₋₆ aliphatic; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; and a 5-6 membered monocyclic heteroaryl ring having 1-4 heteroatoms independently selected from nitrogen, oxygen, and sulfur;
b is 0, 1, 2, or 3;
c is 0, 1, 2, or 3;
each R^{A} is independently selected from deuterium, a C₁₋₆ aliphatic group substituted with 0, 1, 2, or 3 groups independently selected from halogen, deuterium, —CN, —NR₂, and —OR; a 5-6 membered monocyclic heteroaromatic ring having 1-4 heteroatoms independently selected from nitrogen, oxygen, and sulfur substituted with 0, 1, 2, or 3 groups independently selected from halogen, C₁₋₃ alkyl, C₁₋₃ haloalkyl, —CN, —NR₂, and —OR; a 3-8 membered saturated or partially unsaturated monocyclic heterocyclic ring having 1-2 heteroatoms independently selected from nitrogen, oxygen, and sulfur substituted with 0, 1, 2, or 3 groups independently selected from halogen, C₁₋₃ alkyl, C₁₋₃ haloalkyl, —CN, —NR₂, and —OR; a 3-8 membered saturated or partially unsaturated monocyclic carbocyclic ring substituted with 0, 1, 2, or 3 groups independently selected from halogen, C₁₋₃ alkyl, C₁₋₃ haloalkyl, —CN, —NR₂, and —OR; a 4-12 membered saturated or partially unsaturated bicyclic ring system that is fused, bridged, or spirocyclic selected from carbocyclyl and heterocyclyl (having 1-4 heteroatoms independently selected from nitrogen, oxygen, and sulfur), wherein said carbocyclyl or heterocyclyl is substituted with 0, 1, 2, or 3 groups independently selected C₁₋₃ alkyl, C₁₋₃ alkoxy, C₁₋₃ haloalkyl, C₁₋₃ hydroxyalkyl, halogen, —CN, —OR, and —NR₂; halogen; —CN; —NO₂; —OR; —SR; —NR₂; —S(O)₂R; —S(O)₂NR₂; —S(O)R; —S(O)NR₂; —C(O)R; —C(O)OR; —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —S(NR)(O)R; —N(R)S(O)₂NR₂; and —N(R)S(O)₂R; or
two R^{A} on the same atom together form oxo or a cyclic group selected from:
3-8 membered saturated or partially unsaturated monocyclic carbocyclic ring; and
a 3-8 membered saturated or partially unsaturated monocyclic heterocyclic ring having 1-2 heteroatoms independently selected from nitrogen, oxygen, and sulfur;
wherein the cyclic group formed by two R^{A} on the same atom is substituted with 0, 1, 2, or 3 groups independently selected from a C₁₋₆ aliphatic group substituted with 0, 1, 2, or 3 groups independently selected from halogen, —CN, —NR₂, and —OR; halogen; —CN; —NO₂; —OR; —SR; —NR₂; —S(O)₂R; —S(O)₂NR₂; —S(O)R; —S(O)NR₂; —C(O)R; —C(O)OR; —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —N(R)S(O)₂NR₂; and —N(R)S(O)₂R; or
two R^{A} on adjacent atoms form a cyclic group selected from:
3-8 membered saturated or partially unsaturated monocyclic carbocyclic ring;
a 3-8 membered saturated or partially unsaturated monocyclic heterocyclic ring having 1-2 heteroatoms independently selected from nitrogen, oxygen, and sulfur;
phenyl; and
a 5-6 membered heteroaryl ring having 1-4 heteroatoms independently selected from nitrogen, oxygen, and sulfur,
wherein the cyclic group formed by two R^{A} on adjacent atoms is substituted with 0, 1, 2, or 3 groups independently selected from a C₁₋₆ aliphatic group substituted with 0, 1, 2, or 3 groups independently selected from halogen, —CN, —NR₂, and —OR; halogen; —CN; —NO₂; —OR; —SR; —NR₂; —S(O)₂R; —S(O)₂NR₂; —S(O)R; —S(O)NR₂; —C(O)R; —C(O)OR; —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —N(R)S(O)₂NR₂; and —N(R)S(O)₂R;
each R is independently hydrogen, —C(O)N(CH₃)₂, —C(O)₂CH₃, —C(O)₂C(CH₃)₃, —C(O)₂CH(CH₃)₂, —S(O)₂CH₃, an optionally substituted C₁₋₆ aliphatic group, an optionally substituted cyclic group, or an optionally substituted C₁₋₆ alkyl-cyclic group, wherein the cyclic group is selected from:
an optionally substituted phenyl;
an optionally substituted 3-7 membered saturated or partially unsaturated carbocyclic ring;
an optionally substituted 3-7 membered saturated or partially unsaturated heterocyclic ring having 1-2 heteroatoms independently selected from nitrogen, oxygen, and sulfur;
an optionally substituted 5-6 membered heteroaryl ring having 1-4 heteroatoms independently selected from nitrogen, oxygen, and sulfur;
an optionally substituted 8-10 membered bicyclic heteroaryl ring having 1-4 heteroatoms independently selected from nitrogen, oxygen, and sulfur;
an optionally substituted 7-12 membered saturated or partially unsaturated bicyclic heterocyclic ring having 1-4 heteroatoms independently selected from nitrogen, oxygen, and sulfur;
an optionally substituted 5-8 membered saturated or partially unsaturated bridged bicyclic ring having 0-3 heteroatoms independently selected from nitrogen, oxygen, and sulfur;
an optionally substituted 6-10 membered saturated or partially unsaturated spirocyclic ring having 0-3 heteroatoms independently selected from nitrogen, oxygen, and sulfur; and
an optionally substituted 6-11 membered saturated or partially unsaturated bicyclic ring having 0-2 heteroatoms independently selected from nitrogen, oxygen, and sulfur; and/or
two R groups on the same atom are taken together with the same atom to form a cyclic group selected from:
an optionally substituted 4-7 membered saturated or partially unsaturated carbocyclyl;
an optionally substituted 4-7 membered saturated or partially unsaturated heterocyclyl having 1-3 heteroatoms independently selected from nitrogen, oxygen, and sulfur; and
an optionally substituted 5-6 membered heteroaryl ring having 1-3 heteroatoms independently selected from nitrogen, oxygen, and sulfur,
wherein the optionally substituted R moiety is substituted with 0, 1, 2, 3, or 4 R^{Sub};
each R^{Sub} is independently selected from C₁₋₆ aliphatic group substituted with 0, 1, 2, or 3 groups independently selected from halogen, —CN, —N(R^{N})₂, and —O R^{N}; halogen; —CN; —NO₂; —OR^{N}; —SR^{N}; —N(R^{N})₂; —S(O)₂R^{N}; —S(O)₂N(R^{N})₂; —S(O)R^{N}; —S(O)N(R^{N})R₂; —C(O)R^{N}; —C(O)OR^{N}; —C(O)N(R^{N})₂; —C(O)N(R^{N})OR^{N}; —OC(O)R^{N}; —OC(O)N(R^{N})₂; —N(R^{N})C(O)OR^{N}; —N(R^{N})C(O)R^{N}; —N(R^{N})C(O)N(R^{N})₂; —N(R^{N})C(NRN)N(R^{N})₂; —N(R^{N})S(O)₂N(R^{N})₂; and —N(R^{N})S(O)₂R^{N}; and each R^{N} is independently selected from H and C₁₋₃ alkyl.

2. The compound of claim 1, wherein said compound is of any one of the following formulae I-a, 1-b, or I-c: 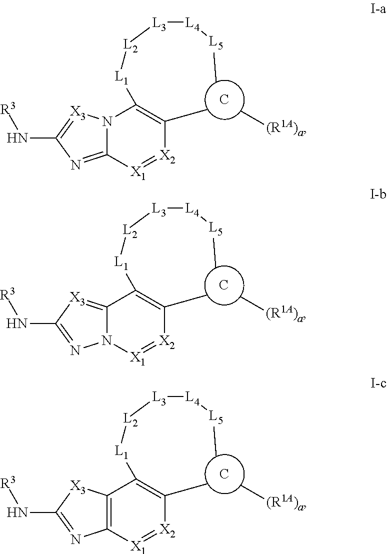
or a pharmaceutically acceptable salt thereof, wherein:
Ring C is a 5-membered monocyclic heteroaryl having 1-3 heteroatoms independently selected from nitrogen, oxygen, and sulfur, and
X₁ and X₂ are independently selected from N, CF, CCl, CCH₃, and CH, provided that only one of X₁ and X₂ may be N.

3. (canceled)

4. The compound of claim 1, wherein Ring C is

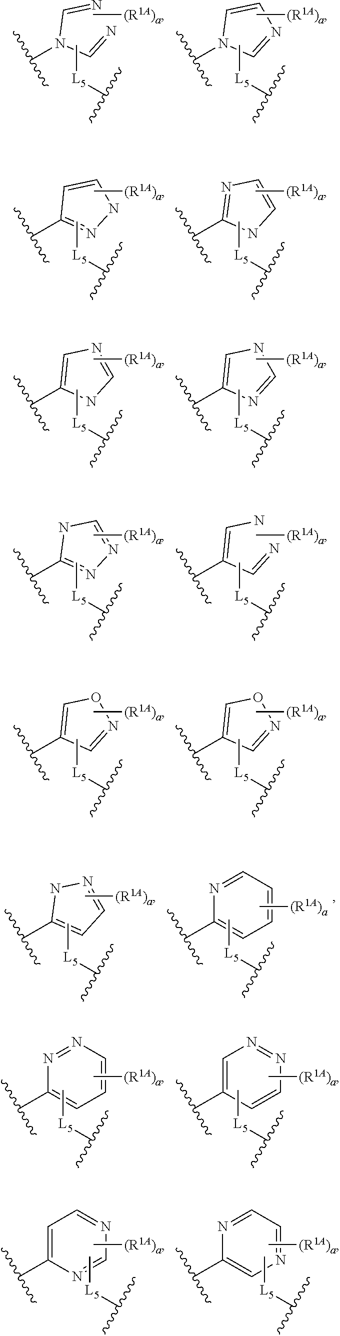

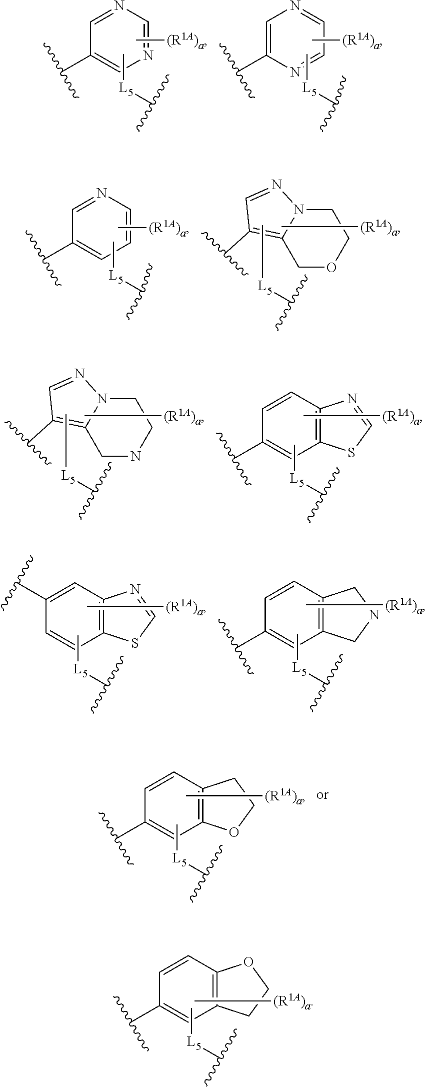

5. The compound of claim 1, wherein said compound is a compound of formulae II-a, II-b, II-c, II-d, or II-e: 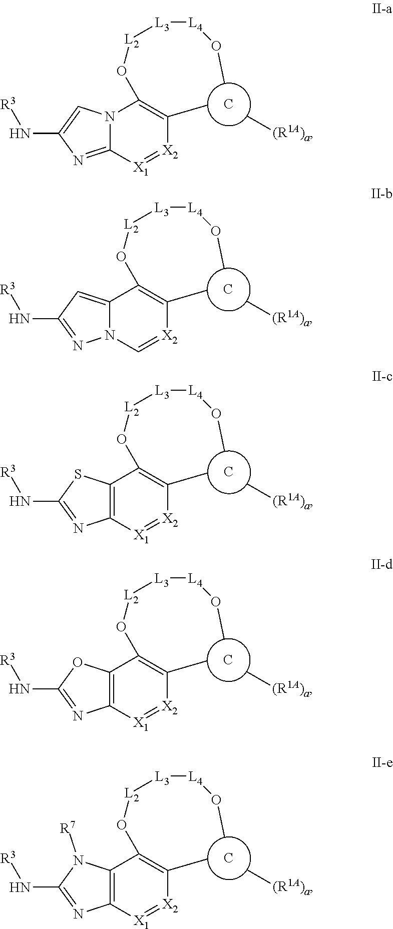
or a pharmaceutically acceptable salt thereof, wherein:
X₁ and X₂ are independently selected from N, CF, CCl, and CH.

6. The compound of claim 1, wherein said compound is a compound of formulae III-a, III-b, III-c, III-d, or III-e: 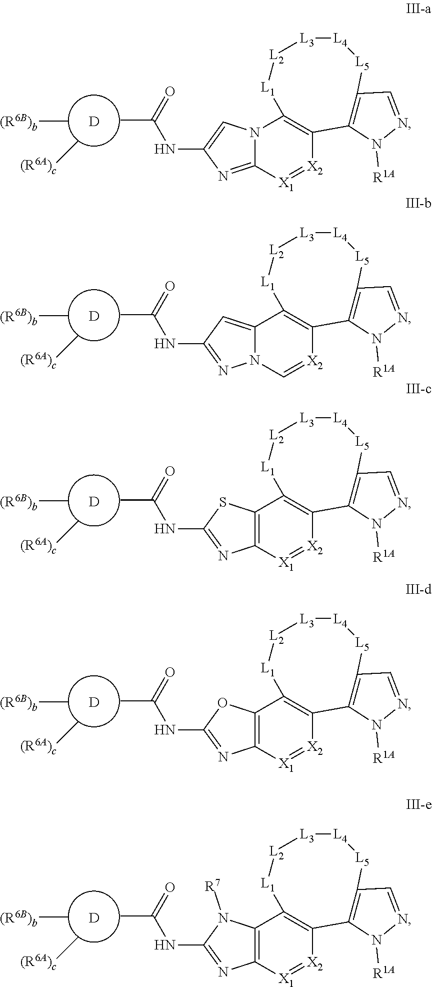
or a pharmaceutically acceptable salt thereof, wherein:
X₁ and X₂ are independently selected from N, CF, CCl, and CH.

7. The compound of claim 1, wherein said compound is a compound of formulae IV-a, IV-b, IV-c, IV-d, or VI-e: 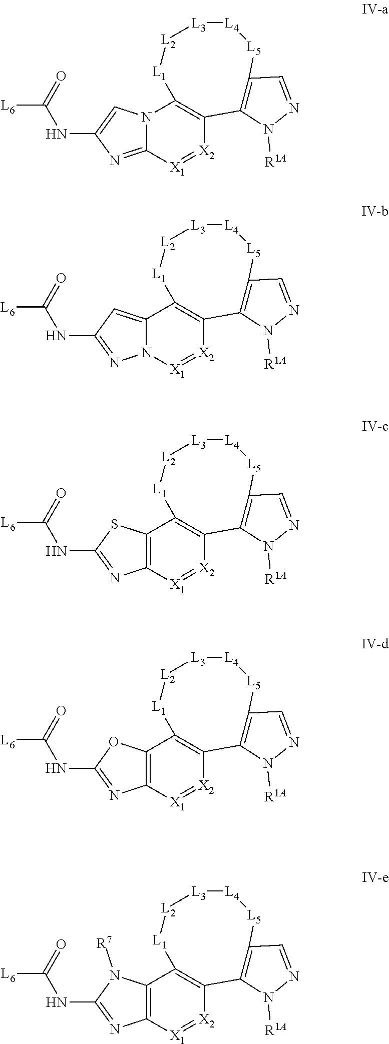
or a pharmaceutically acceptable salt thereof, wherein:
X₁, and X₂ are independently selected from CH, N, CF, CCl, CCH₃, and
L₆ is selected from the group consisting of

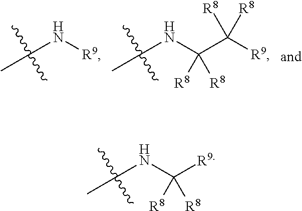

8. The compound of claim 1, wherein L₁ is —CH₂— or —O— and L₅ is —CH₂— or —O—.

9. The compound of claim 1, wherein said compound of formula I is a compound of formulae V-a, V-b, V-c, V-d, or V-e: 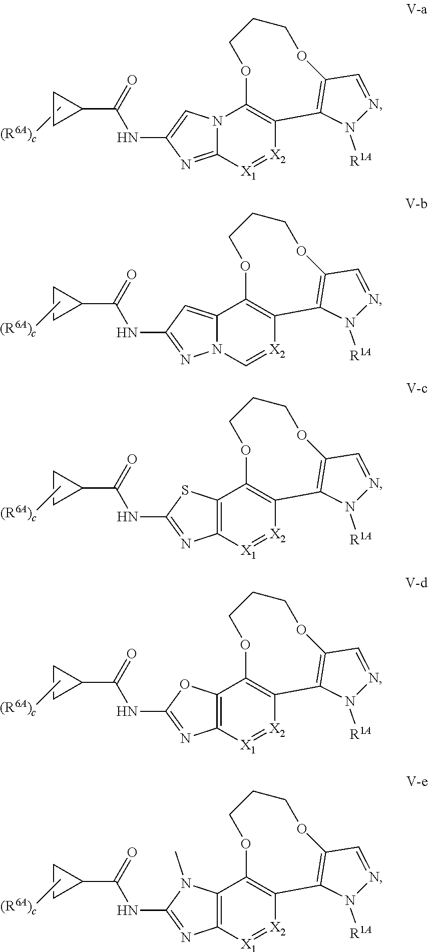
or a pharmaceutically acceptable salt thereof, wherein:
X₁ and X₂ are independently selected from CH, N, CF, CCl, and CCH₃.

10. The compound of claim 1, where R^{6A} is

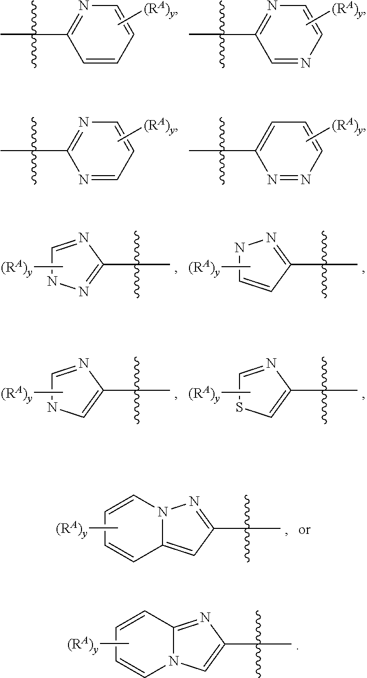

11. The compound of claim 1, wherein the compound is selected from those depicted in Table 1, or a pharmaceutically acceptable salt thereof.

12. A pharmaceutical composition comprising a compound of claim 1, or a pharmaceutically acceptable salt thereof, and a pharmaceutically acceptable carrier, adjuvant, or vehicle.

13. (canceled)

14. A method of inhibiting SIK2 in a biological sample, comprising contacting the sample with the compound of claim 1, or a pharmaceutically acceptable salt thereof.

15. A method of treating an SIK2-mediated disorder, disease, or condition in a patient, comprising administering to said patient the compound of claim 1, or a pharmaceutically acceptable salt thereof.

16. The method of claim 15, wherein the SIK2-mediated disorder is selected from inflammatory diseases, autoinflammatory diseases, autoimmune diseases, proliferative diseases, fibrotic diseases, transplantation rejection, diseases involving impairment of cartilage turnover, congenital cartilage malformation, diseases involving impairment of bone turnover, diseases associated with hypersecretion of TNFα, interferons, IL-6, IL-12 and/or IL-23, respiratory diseases, endocrine and/or metabolic diseases, cardiovascular diseases, dermatological diseases, and abnormal angiogenesis associated diseases.

17. The method of claim 15, wherein the SIK2-mediated disorder is a cancer selected from ovarian cancer, breast cancer, acute myeloid leukemia, and multiple myeloma.

18. The method of claim 15, wherein the SIK2-mediated disorder is inflammatory bowel disease, diabetes, a skin pigmentation disease, osteoporosis, osteoarthritis, a musculoskeletal disease, or rheumatoid arthritis.
